(19) United States
(12) Patent Application Publication
(10) Pub. No.: US 2018/0338980 A1
(43) Pub. Date: Nov. 29, 2018

(54) AROMATIC SULFONAMIDE DERIVATIVES

(71) Applicant: BAYER PHARMA AKTIENGESELLSCHAFT, Berlin (DE)

(72) Inventors: Stefan WERNER, Berlin (DE); Stefanie MESCH, Berlin (DE); Nico BRÄUER, Falkensee (DE); Elisabeth POOK, Wuppertal (DE); Henrik DAHLLÖF, Uppsala (SE); Reinhard NUBBEMEYER, Berlin (DE); Maren OSMERS, Dallgow-Döberitz (DE); Bernd KALTHOF, Wuppertal (DE)

(21) Appl. No.: 15/580,830

(22) PCT Filed: Jun. 7, 2016
(86) PCT No.: PCT/EP2016/062841

§ 371 Date: Dec. 8, 2017

(30) Foreign Application Priority Data
Jun. 10, 2015 (EP) 15171318.7

(51) Int. Cl. A61K 31/5377; C07D 413/10; A61K 31/444; A61K 31/496; C07D 401/10

(52) U.S. Cl. CPC A61K 31/5377; C07D 413/10; C07D 401/10; A61K 31/496; A61K 31/444

(57) ABSTRACT

Substituted aromatic sulfonamides of formula (I) pharmaceutical compositions and combinations comprising said compounds and the use of said compounds for manufacturing a pharmaceutical composition for the treatment or prophylaxis of a disease.

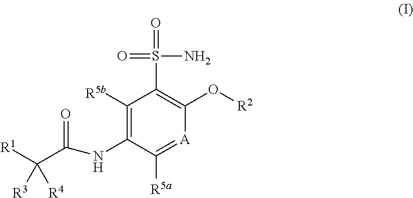

### FIELD OF APPLICATION OF THE INVENTION

The invention relates to substituted aromatic sulfonamides of formula (I) as described and defined herein, pharmaceutical compositions and combinations comprising said compounds and to the use of said compounds for manufacturing a pharmaceutical composition for the treatment or prophylaxis of a disease. The present invention, as described and defined herein, relates to pharmaceutical compositions and combinations comprising an active ingredient which is an antagonist or a negative allosteric modulator of P2X4. The use of such compounds for manufacturing a pharmaceutical composition for the treatment or prophylaxis of a disease, in particular in mammals, such as but not limited to diseases associated with pain, or for the treatment or prophylaxis of pain syndromes (acute and chronic), inflammatory-induced pain, neuropathic pain, pelvic pain, cancer-associated pain, endometriosis-associated pain as well as endometriosis as such, cancer as such, and proliferative diseases as such like endometriosis, as a sole agent or in combination with other active ingredients.

### BACKGROUND OF THE INVENTION

Chronic inflammatory pain such as in, but not limited to, conditions of endometriosis and adenomyosis, arises as a consequence of inflammatory responses mounted by the immune system following tissue damage and generally persists long after the initial injury has healed. Since a large percentage of patients with inflammatory diseases do not respond adequately to currently available analgesic drugs or suffer from intolerable side effects, investigation of alternative treatments for inflammatory conditions/disorders is warranted.

Adenosine triphosphate ATP is widely recognized as important neurotransmitter implicated in various physiological and pathophysiological roles by acting through different subtypes of purinergic receptors (Burnstock 1993, Drug Dev Res 28:196-206; Burnstock 2011, Prog Neurobiol 95:229-274). To date, seven members of the P2X family have been cloned, comprising P2X1-7 (Burnstock 2013, Front Cell Neurosci 7:227). The P2X4 receptor is a ligand-gated ion channel that is expressed on a variety of cell types largely those known to be involved in inflammatory/immune processes specifically including monocytes, macrophages, mast cells and microglia cells (Wang et al., 2004, BMC Immunol 5:16; Brone et al., 2007 Immunol Lett 113:83-89). Activation of P2X4 by extracellular ATP is known, amongst other things, to lead to release of pro-inflammatory cytokines and prostaglandins (PGE2) (Bo et al., 2003 Cell Tissue Res 313:159-165; Ulmann et al., 2010, EMBO Journal 29:2290-2300; de Ribero Vaccari et al., 2012, J Neurosci 32:3058-3066). Numerous lines of evidence in the literature using animal models implicate P2X4 receptor in nociception and pain. Mice lacking the P2X4 receptor do not develop pain hypersensitivity in response to numerous inflammatory challenges such as complete Freunds Adjuvant, carrageenan or formalin (Ulmann et al., 2010, EMBO Journal 29:2290-2300). In addition, mice lacking the P2X4R do not develop mechanical allodynia after peripheral nerve injury, indicating an important role of P2X4 also in neuropathic pain conditions (Tsuda et al., 2009, Mol Pain 5:28; Ulmann et al., 2008, J Neurocsci 28:11263-11268).

Besides the prominent role of P2X4 in acute and chronic pain-related diseases (Trang and Salter, 2012, Purinergic Signalling 8:621-628.), P2X4 is considered as a critically important mediator of inflammatory diseases such as, respiratory diseases (e.g. asthma, COPD), lung diseases including fibrosis, bone metabolism, cancer and atherosclerosis (Burnstock et al., 2012 Pharmacol Rev. 64:834-868).

EP 2 597 088 A1 describes P2X4 receptor antagonists and in particular a diazepine derivative of formula (III) or a pharmacologically acceptable salt thereof. Said document further disclosed the use of P2X4 receptor antagonist diazepine derivatives represented by the formula (I), (II), (III), or its pharmacologically acceptable salt, which shows P2X4 receptor antagonism, being effective as an agent for prevention or treatment of nociceptive, inflammatory, and neuropathic pain. In more detail, EP 2 597 088 A1 describes P2X4 receptor antagonists being effective as a preventive or therapeutic agent for pain caused by various cancers, diabetic neuritis, viral diseases such as herpes, and osteoarthritis. The preventive or therapeutic agent according to EP 2 597 088 A1 can also be used in combination with other agents such as opioid analgesic (e.g., morphine, fentanyl), sodium channel inhibitor (e.g., novocaine, lidocaine), or NSAIDs (e.g., aspirin, ibuprofen). The P2X4 receptor antagonist used for pain caused by cancers can be also used in combination with a carcinostatic such as a chemotherapic. Further P2X4 receptor antagonists and their use are disclosed in WO02015005467 and WO02015005468.

“Discovery and characterization of novel, potent and selective P2X4 receptor antagonists for the treatment of pain” was presented at the Society for Neuroscience Annual Meeting 2014 (Carrie A Bowen et al.; poster N. 241.1) Said poster describes the methods to identify novel, potent and selective small-molecule antagonists that inhibit P2X4 across species, and how to evaluate selected compounds in experimental models of neuropathic and inflammatory pain. In particular a method for human, rat, mouse P2X4R Flipr-based screening, a human P2X4R electrophysiology assay, a suitable mouse neuropathy model and a mouse inflammation model were described.

WO1998025893 provides novel arylsulfonamides. These compounds have been found to inhibit phospholipase A2 activity, in particular cPLA2 (cytosolic phospholipase A2). Additionally, the compounds inhibit the release of cytokines in stimulated cells. Still further, the compounds have been found to inhibit neurodegeneration in a mammalian neuronal cell population.

WO2009138758 describes novel pharmaceutically-useful bis-aryl compounds, which compounds are useful as inhibitors of the production of leukotrienes, such as leukotriene C4. The compounds are of potential utility in the treatment of respiratory and/or inflammatory diseases. The invention also relates to the use of such compounds as medicaments, to pharmaceutical compositions containing them, and to synthetic routes for their production.

WO2009136889 describes substituted isoindoles, which are vascular endothelial growth factor receptor (VEGFR) inhibitors, pharmaceutical compositions containing the same, and methods of using the same as anti-tumor agents for treatment of cancer (e.g., breast, colorectal, lung, prostate, and ovarian).

WO2013192517 provides compounds useful for inhibiting fungal or parasitic growth, pharmaceutically acceptable salts thereof, and pharmaceutical compositions thereof.

The compounds are useful as inhibitors of glycosylphosphatidylinositol (GPI)-anchor biosynthesis, in particular, as inhibitors of fungal GwtI activity.

There is no reference in the state of the art about substituted aromatic sulfonamides of general formula (I) as described and defined herein and to the use of said compounds for manufacturing a pharmaceutical composition for the treatment or prophylaxis of a disease, particularly to the use of substituted aromatic sulfonamides of general formula (I) for the treatment or prophylaxis of diseases associated with pain, or for the treatment or prophylaxis of pain syndromes (acute and chronic), inflammatory-induced pain, neuropathic pain, pelvic pain, cancer-associated pain, endometriosis-associated pain as well as endometriosis as such, cancer as such, and proliferative diseases as such like endometriosis, as a sole agent or in combination with other active ingredients.

Therefore, the inhibitors of P2X4 of the current invention represent valuable compounds that should complement therapeutic options either as single agents or in combination with other drugs.

### DESCRIPTION OF THE INVENTION

The present invention relates to a compound of formula (I)

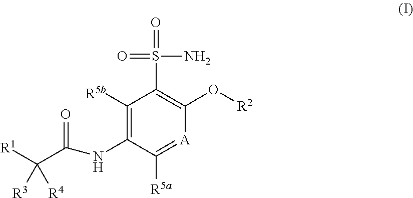

in which:
A represents CR⁵ or N;
R¹ represents a group selected from:

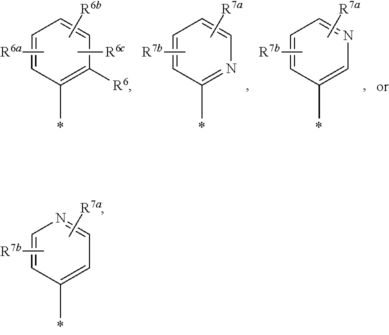

wherein * indicates the point of attachment of said group with the rest of the molecule;
R² represents C₃-C₆-cycloalkyl, C₃-C₆-cycloalkyl-C₁-C₄-alkyl, 4- to 6-membered heterocycloalkyl, 4- to 6-membered heterocycloalkyl-C₁-C₄-alkyl, phenyl, phenyl-C₁-C₄-alkyl, heteroaryl or heteroaryl-C₁-C₄-alkyl,
   wherein said groups are optionally substituted one to four times with R¹¹, being, independently from each other, the same or different, or
substituted one time with R¹¹ and optionally one to two times with R¹¹ being independently from each other, the same or different, or
substituted with two adjacent substituents R¹¹ which together represent a methylendioxy group to form a 5-membered ring or
substituted with one to five deuterium atoms and optionally one to two times with R¹¹ being, independently from each other, the same or different, or
R² represents branched (C₁-C₄-alkyl)-C₁-C₄-alkyl;
R³ represents hydrogen, deuterium, fluoro or methyl;
R⁴ represents hydrogen, deuterium or fluoro;
R⁵, R^{5a} and R^{5b} are the same or different and represent, independently from each other, hydrogen, halogen, C₁-C₄-alkyl, C₁-C₄-haloalkyl, C₁-C₄-alkoxy or C₁-C₄-haloalkoxy;
R⁶, R^{6a}, R^{6b} and R^{6c} are the same or different and represent, independently from each other, respectively
R⁶ hydrogen, halogen, cyano, C₁-C₄-alkyl, C₁-C₄-haloalkyl, C₁-C₄-alkoxy, C₁-C₄-haloalkoxy, HO—(C₂-C₄-alkoxy)-, (C₁-C₄-alkoxy)-(C₂-C₄-alkoxy)- or F₃C—S—;
R^{6a} hydrogen, halogen, hydroxy, nitro, cyano, C₁-C₄-alkyl, C₃-C₆-cycloalkyl, C₁-C₄-haloalkyl, C₁-C₄-alkoxy, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—;
R^{6b} hydrogen, halogen, hydroxy, nitro, cyano, C₁-C₄-alkyl, C₃-C₆-cycloalkyl, C₁-C₄-haloalkyl, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—; or
R^{6a} and R^{6b} adjacent to each other together represent a group selected from —O—CH₂—CH₂—, —O—CH₂—O— or —O—CH₂—CH₂—O—;
R^{6c} hydrogen or halogen;
R^{7a} and R^{7b} are the same or different and represent, independently from each other, hydrogen, hydroxy, halogen, C₁-C₄-alkyl or C₁-C₄-haloalkyl;
R⁸ represents, independently from each respective occurrence, C₁-C₆-alkyl, C₁-C₄-alkoxy-C₁-C₄-alkyl, C₃-C₆-cycloalkyl or C₁-C₄-haloalkyl;
R⁹ and R¹⁰ are the same or different and represent, independently from each other, hydrogen, C₁-C₄-alkyl, C₃-C₆-cycloalkyl, C₁-C₄-haloalkyl, (C₁-C₄-alkoxy)-(C₂-C₄-alkyl), phenyl or heteroaryl, wherein said phenyl and heteroaryl groups are optionally substituted one to three times, independently from each other, with hydrogen, halogen, C₁-C₄-alkyl, C₁-C₄-haloalkyl, C₁-C₄-alkoxy or C₁-C₄-haloalkoxy,
R^{9a} and R^{10a} together with the nitrogen atom to which they are attached form a 4- to 6-membered nitrogen containing heterocyclic ring, said ring optionally containing one additional heteroatom selected from O, NH, NR^{a} in which R^{a} represents a C₁-C₆-alkyl- or C₁-C₆-haloalkyl- group or S and being optionally substituted, one to three times, independently from each other, with halogen or C₁-C₄-alkyl;
R¹¹ represents, independently from each other, halogen, hydroxy, nitro, cyano, C₁-C₄-alkyl, C₂-C₄-alkenyl, C₁-C₄-haloalkyl, C₁-C₄-hydroxyalkyl, C₁-C₄-alkoxy, C₁-C₄-haloalkoxy, HO—(C₂-C₄-alkoxy)-, (C₁-C₄-alkoxy)-(C₂-C₄-alkoxy)-, (C₁-C₄-haloalkyl)-S—, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—;
R^{11a} represents a group selected from C₃-C₆-cycloalkyl, morpholino, R^{9a}R^{10a}N—; R^{9a}R^{10a}N—C(O)—; a 5- to 6-membered heteroaryl, which is optionally substituted with methyl, or
   represents:

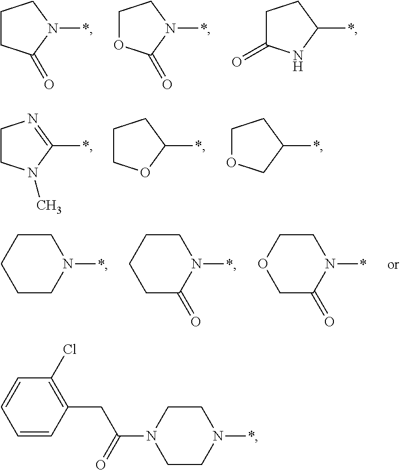

wherein * indicates the point of attachment of said group with the rest of the molecule; or
an N-oxide, a salt, a hydrate, a solvate, a tautomer or a stereoisomer of said compound, or a salt of said N-oxide, tautomer or stereoisomer.

In a second aspect, the invention relates in particular to compounds of formula (Ia),

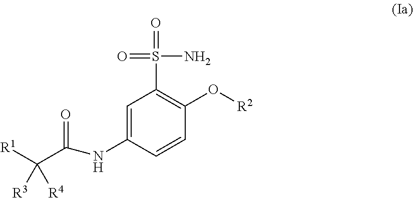

wherein
R¹ represents a group selected from:

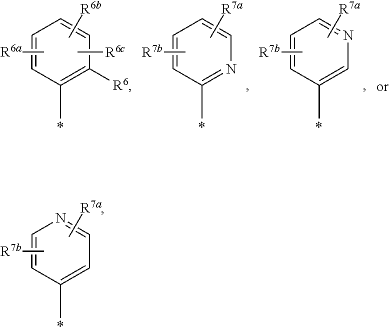

wherein * indicates the point of attachment of said group with the rest of the molecule;
R² represents C₃-C₆-cycloalkyl, C₃-C₆-cycloalkyl-C₁-C₄-alkyl, 4- to 6-membered heterocycloalkyl, 4- to 6-membered heterocycloalkyl-C₁-C₄-alkyl, phenyl, phenyl-C₁-C₄-alkyl, heteroaryl or heteroaryl-C₁-C₄-alkyl,
   wherein said groups are optionally substituted one to four times with R¹¹, being, independently from each other, the same or different, or
   substituted one time with R^{11a} and optionally one to two times with R¹¹ being independently from each other, the same or different, or
   substituted with two adjacent substituents R¹¹ which together represent a methylendioxy group to form a 5-membered ring or
   substituted with one to five deuterium atoms and optionally one to two times with R¹¹ or R¹¹ being, independently from each other, the same or different; or
R² represents branched (C₁-C₄-alkyl)-C₁-C₄-alkyl;
R³ represents hydrogen, deuterium, fluoro or methyl;
R⁴ represents hydrogen, deuterium or fluoro;
R⁶, R^{6a}, R^{6b} and R^{6c} are the same or different and represent, independently from each other, respectively
R⁶ hydrogen, halogen, cyano, C₁-C₄-alkyl, C₁-C₄-haloalkyl, C₁-C₄-alkoxy, C₁-C₄-haloalkoxy, HO—(C₂-C₄-alkoxy)-, (C₁-C₄-alkoxy)-(C₂-C₄-alkoxy)- or F₃C—S—;
R^{6a} hydrogen, halogen, hydroxy, nitro, cyano, C₁-C₄-alkyl, C₃-C₆-cycloalkyl, C₁-C₄-haloalkyl, C₁-C₄-alkoxy, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—;
R^{6b} hydrogen, halogen, hydroxy, nitro, cyano, C₁-C₄-alkyl, C₃-C₆-cycloalkyl, C₁-C₄-haloalkyl, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—; or
R^{6a} and R^{6b} adjacent to each other together represent a group selected from —O—CH₂—CH₂—, —O—CH₂—O— or —O—CH₂—CH₂—O—;
R^{6c} hydrogen or halogen;
R^{7a} and R^{7b} are the same or different and represent, independently from each other, hydrogen, hydroxy, halogen, C₁-C₄-alkyl or C₁-C₄-haloalkyl;
R⁸ represents, independently from each respective occurrence, C₁-C₆-alkyl, C₁-C₄-alkoxy-C₁-C₄-alkyl, C₃-C₆-cycloalkyl or C₁-C₄-haloalkyl;
R⁹ and R¹⁰ are the same or different and represent, independently from each other, hydrogen, C₁-C₄-alkyl, C₃-C₆-cycloalkyl, C₁-C₄-haloalkyl, (C₁-C₄-alkoxy)-(C₂-C₄-alkyl), phenyl or heteroaryl, wherein said phenyl and heteroaryl groups are optionally substituted one to three times, independently from each other, with hydrogen, halogen, C₁-C₄-alkyl, C₁-C₄-haloalkyl, C₁-C₄-alkoxy or C₁-C₄-haloalkoxy,
R^{9a} and R^{10a} together with the nitrogen atom to which they are attached form a 4- to 6-membered nitrogen containing heterocyclic ring, said ring optionally containing one additional heteroatom selected from O, NH, NR^{a} in which R^{a} represents a C₁-C₆-alkyl- or C₁-C₆-haloalkyl- group, or S and being optionally substituted, one to three times, independently from each other, with halogen or C₁-C₄-alkyl and;
R¹¹ represents, independently from each other, halogen, hydroxy, nitro, cyano, C₁-C₄-alkyl, C₂-C₄-alkenyl, C₁-C₄-haloalkyl, C₁-C₄-hydroxyalkyl, C₁-C₄-alkoxy, C₁-C₄-haloalkoxy, HO—(C₂-C₄-alkoxy)-, (C₁-C₄-alkoxy)-(C₂-C₄-alkoxy)-, (C₁-C₄-haloalkyl)-S—, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—;
R^{11a} represents a group selected from C₃-C₆-cycloalkyl, morpholino, R^{9a}R^{10a}N—; R^{9a}R^{10a}N—C(O)—; a 5- to 6-membered heteroaryl, which is optionally substituted with methyl or
   represents:

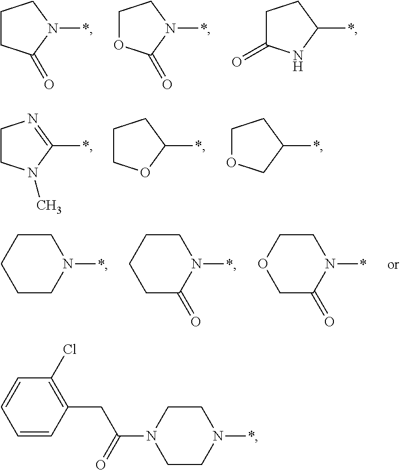

wherein * indicates the point of attachment of said group with the rest of the molecule; or
   an N-oxide, a salt, a hydrate, a solvate, a tautomer or a stereoisomer of said compound, or a salt of said N-oxide, tautomer or stereoisomer.

In a third aspect, the invention relates in particular to compounds of formula (Ib)

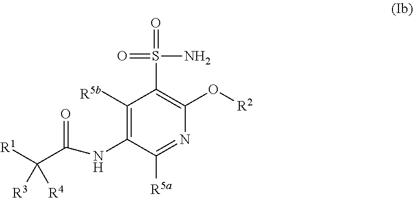

wherein
R¹ represents a group selected from:

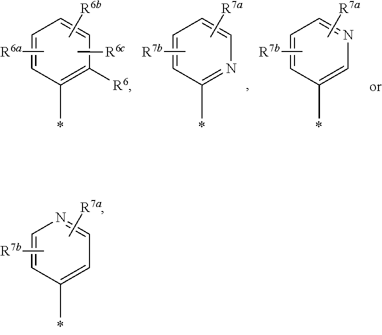

wherein * indicates the point of attachment of said group with the rest of the molecule;
R² represents C₃-C₆-cycloalkyl, C₃-C₆-cycloalkyl-C₁-C₄-alkyl, 4- to 6-membered heterocycloalkyl, 4- to 6-membered heterocycloalkyl-C₁-C₄-alkyl, phenyl, phenyl-C₁-C₄-alkyl, heteroaryl or heteroaryl-C₁-C₄-alkyl,
   wherein said groups are optionally substituted one to four times with R¹¹, being, independently from each other, the same or different, or
   substituted one time with R^{11a} and optionally one to two times with R¹¹, being independently from each other, the same or different, or
   substituted with two adjacent substituents R¹¹ which together represent a methylendioxy group to form a 5-membered ring or
   substituted with one to five deuterium atoms and optionally one to two times with R¹¹ being, independently from each other, the same or different; or
R² represents branched (C₁-C₄-alkyl)-C₁-C₄-alkyl;
R³ represents hydrogen, fluoro or methyl;
R⁴ represents hydrogen or fluoro;
R^{5a} and R^{5b} are the same or different and represent, independently from each other, hydrogen, halogen, C₁-C₄-alkyl, C₁-C₄-haloalkyl, C₁-C₄-alkoxy or C₁-C₄-haloalkoxy;
R⁶, R^{6a}, R^{6b} and R^{6c} are the same or different and represent, independently from each other, respectively
R⁶ hydrogen, halogen, cyano, C₁-C₄-alkyl, C₁-C₄-haloalkyl, C₁-C₄-alkoxy, C₁-C₄-haloalkoxy, HO—(C₂-C₄-alkoxy)-, (C₁-C₄-alkoxy)-(C₂-C₄-alkoxy)- or F₃C—S—;
R^{6a} hydrogen, halogen, hydroxy, nitro, cyano, C₁-C₄-alkyl, C₃-C₆-cycloalkyl, C₁-C₄-haloalkyl, C₁-C₄-alkoxy, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R—O—C(O)—, R⁹R¹⁰N—C(O)— or (C₁-C₄-alkyl)-SO₂—;
R^{6b} hydrogen, halogen, hydroxy, nitro, cyano, C₁-C₄-alkyl, C₃-C₆-cycloalkyl, C₁-C₄-haloalkyl, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—; or
R^{6a} and R^{6b} adjacent to each other together represent a group selected from —O—CH₂—CH₂—, —O—CH₂—O— or —O—CH₂—CH₂—O—; R^{6c} hydrogen or halogen;
R^{7a} and R^{7b} are the same or different and represent, independently from each other, hydrogen, hydroxy, halogen, C₁-C₄-alkyl or C₁-C₄-haloalkyl;
R⁸ represents, independently from each respective occurrence, C₁-C₆-alkyl, C₁-C₄-alkoxy-C₁-C₄-alkyl, C₃-C₆-cycloalkyl or C₁-C₄-haloalkyl;
R⁹ and R¹⁰ are the same or different and represent, independently from each other, hydrogen, C₁-C₄-alkyl, C₃-C₆-cycloalkyl, C₁-C₄-haloalkyl, (C₁-C₄-alkoxy)-(C₂-C₄-alkyl), phenyl or heteroaryl, wherein said phenyl and heteroaryl groups are optionally substituted one to three times, independently from each other, with hydrogen, halogen, C₁-C₄-alkyl, C₁-C₄-haloalkyl, C₁-C₄-alkoxy or C₁-C₄-haloalkoxy,
R^{9a} and R^{10a} together with the nitrogen atom to which they are attached form a 4- to 6-membered nitrogen containing heterocyclic ring, said ring optionally containing one additional heteroatom selected from O, NH, NR^{a} in which R^{a} represents a C₁-C₆-alkyl- or C₁-C₆-haloalkyl- group, or S and being optionally substituted, one to three times, independently from each other, with halogen or C₁-C₄-alkyl;
R¹¹ represents, independently from each other, halogen, hydroxy, nitro, cyano, C₁-C₄-alkyl, C₂-C₄-alkenyl, C₁-C₄-haloalkyl, C₁-C₄-hydroxyalkyl, C₁-C₄-alkoxy, C₁-C₄-haloalkoxy, HO—(C₂-C₄-alkoxy)-, (C₁-C₄-alkoxy)-(C₂-C₄-alkoxy)-, (C₁-C₄-haloalkyl)-S—, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—;
R^{11a} represents a group selected from C₃-C₆-cycloalkyl, morpholino, R^{9a}R^{10a}N—; R^{9a}R^{10a}N—C(O)—; a 5- to 6-membered heteroaryl, which is optionally substituted with methyl or
   represents:

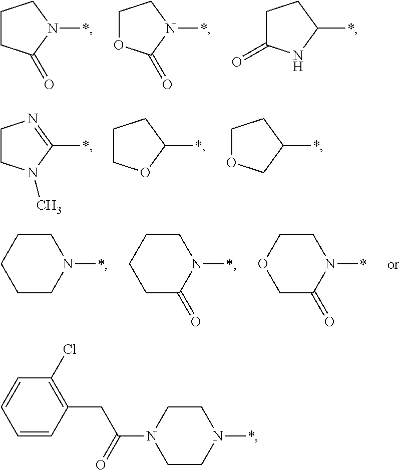

wherein * indicates the point of attachment of said group with the rest of the molecule; or
   an N-oxide, a salt, a hydrate, a solvate, a tautomer or a stereoisomer of said compound, or a salt of said N-oxide, tautomer or stereoisomer.

In a fourth aspect, the invention refers more in particular to compounds of formula (Ia) as described supra, wherein:
R¹ represents a group selected from:

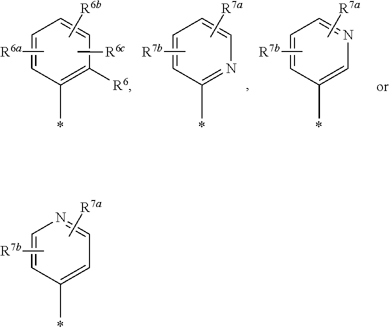

wherein * indicates the point of attachment of said group with the rest of the molecule;
R² represents C₃-C₆-cycloalkyl, C₃-C₆-cycloalkyl-C₁-C₄-alkyl, 4- to 6-membered heterocycloalkyl, 4- to 6-membered heterocycloalkyl-C₁-C₄-alkyl, phenyl, phenyl-C₁-C₄-alkyl, heteroaryl or heteroaryl-C₁-C₄-alkyl,
   wherein said groups are optionally substituted one to four times with R¹¹, being, independently from each other, the same or different, or
   substituted one time with R^{11a} and optionally one to two times with R¹¹ being independently from each other, the same or different, or
   substituted with two adjacent substituents R¹¹ which together represent a methylendioxy group to form a 5-membered ring or
   substituted with one to five deuterium atoms and optionally one to two times with R¹¹ being, independently from each other, the same or different;
R³ represents hydrogen, fluoro or methyl;
R⁴ represents hydrogen or fluoro;
R⁶, R^{6a}, R^{6b} and R^{6c} are the same or different and represent, independently from each other, respectively
R⁶ hydrogen, fluoro, chloro, bromo, cyano, C₁-C₄-alkyl, difluoromethyl, trifluoromethyl, methoxy, ethoxy, difluoromethoxy, trifluoromethoxy, 2-hydroxy-ethoxy, 2-methoxy-ethoxy or F₃C—S—;
R^{6a} hydrogen, fluoro, chloro, bromo, hydroxy, cyano, methyl, difluoromethyl, trifluoromethyl, methoxy, 2-hydroxy-ethoxy, 2-methoxy-ethoxy or R⁹R¹⁰N—C(O)—;
R^{6b} hydrogen, fluoro, chloro or bromo; or
R^{6a} and R^{6b} adjacent to each other together represent a group selected from —O—CH₂—CH₂—, —O—CH₂—O— or —O—CH₂—CH₂—O—;
R^{6c} hydrogen or halogen;
R^{7a} and R^{7b} are the same or different and represent, independently from each other, hydrogen, chloro, methyl, difluoromethyl or trifluoromethyl;
R⁸ represents methyl;
R⁹ and R¹⁰ are the same or different and represent, independently from each other, hydrogen, methyl, cyclopropyl or 2-methoxy-ethyl;
R^{9a} and R^{10a} together with the nitrogen atom to which they are attached form a 4- to 6-membered nitrogen containing heterocyclic ring, said ring optionally containing one additional heteroatom selected from O, NH, NCH₃ or S and being optionally substituted, one to three times, independently from each other, with halogen or methyl;
R¹¹ represents, independently from each other, halogen, hydroxy, nitro, cyano, C₁-C₄-alkyl, C₂-C₄-alkenyl, C₁-C₄-haloalkyl, C₁-C₄-hydroxyalkyl, C₁-C₄-alkoxy, C₁-C₄-haloalkoxy, HO—(C₂-C₄-alkoxy)-, (C₁-C₄-alkoxy)-(C₂-C₄-alkoxy)-, (C₁-C₄-haloalkyl)-S—, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—;
R^{11a} represents a group selected from C₃-C₆-cycloalkyl, morpholino, R^{9a}R^{10a}N—; R^{9a}R^{10a}N—C(O)—; a 5- to 6-membered heteroaryl, which is optionally substituted with methyl or
   represents a group selected from:

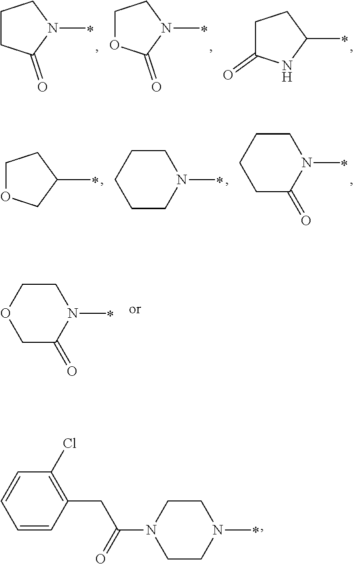

wherein * indicates the point of attachment of said group with the rest of the molecule; or
   an N-oxide, a salt, a hydrate, a solvate, a tautomer or a stereoisomer of said compound, or a salt of said N-oxide, tautomer or stereoisomer.

Furthermore, according to a particular aspect of the present invention, compounds of formula (Ia) as described supra, are those wherein:
R¹ represents a group selected from:

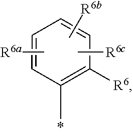

wherein * indicates the point of attachment of said group with the rest of the molecule;
R² represents C₄-C₆-cycloalkyl, C₃-C₆-cycloalkyl-methyl, 4- to 6-membered heterocycloalkyl, 4- to 6-membered heterocycloalkyl-methyl, phenyl, phenyl-C₁-C₂-alkyl, heteroaryl, heteroaryl-methyl wherein said groups are optionally substituted one to four times with R¹¹, being, independently from each other, the same or different, or
   substituted one time with R^{11a} and optionally one to two times with R¹¹ being independently from each other, the same or different, or
   substituted with two adjacent substituents R¹¹ which together represent a methylendioxy group to form a 5-membered ring;
R³ represents hydrogen or methyl;
R⁴ represents a hydrogen;
R⁶, R^{6a} and R^{6b} are the same or different and represent, independently from each other, respectively
R⁶ hydrogen, fluoro, chloro, bromo, cyano, C₁-C₄-alkyl, difluoromethyl, trifluoromethyl, methoxy, ethoxy, difluoromethoxy, trifluoromethoxy, 2-hydroxy-ethoxy, 2-methoxy-ethoxy or F₃C—S—;
R^{6a} hydrogen, fluoro, chloro, bromo, hydroxy, cyano, methyl, difluoromethyl, trifluoromethyl, methoxy, 2-hydroxy-ethoxy, 2-methoxy-ethoxy or R⁹R¹⁰N—C(O)—;
R^{6b} hydrogen, fluoro, chloro or bromo; or
R^{6a} and R^{6b} adjacent to each other together represent a group selected from —O—CH₂—CH₂—, —O—CH₂—O— or —O—CH₂—CH₂—O—;
R^{6c} hydrogen or halogen;
R⁹ and R¹⁰ are the same or different and represent, independently from each other, hydrogen, methyl, cyclopropyl or 2-methoxy-ethyl;
R^{9a} and R^{10a} together with the nitrogen atom to which they are attached form a 4- to 6-membered nitrogen containing heterocyclic ring, said ring optionally containing one additional heteroatom selected from O, NH, NR^{a} in which R^{a} represents a C₁-C₆-alkyl- or C₁-C₆-haloalkyl- group, or S and being optionally substituted, one to three times, independently from each other, with halogen or methyl;
R¹¹ represents, independently from each other, halogen, hydroxy, nitro, cyano, C₁-C₄-alkyl, C₂-C₄-alkenyl, C₁-C₄-haloalkyl, C₁-C₄-hydroxyalkyl, C₁-C₄-alkoxy, C₁-C₄-haloalkoxy, HO—(C₂-C₄-alkoxy)-, (C₁-C₄-alkoxy)-(C₂-C₄-alkoxy)-, (C₁-C₄-haloalkyl)-S—, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—;
R^{11a} represents a group selected from C₃-C₆-cycloalkyl, morpholino, R^{9a}R^{10a}N—; R^{9a}R^{10a}N—C(O)—; a 5- to 6-membered heteroaryl, which is optionally substituted with methyl or
   represents a group selected from:

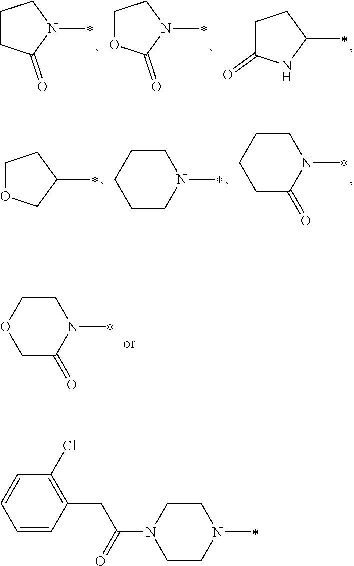

wherein * indicates the point of attachment of said group with the rest of the molecule; or
   an N-oxide, a salt, a hydrate, a solvate, a tautomer or a stereoisomer of said compound, or a salt of said N-oxide, tautomer or stereoisomer.

In particular the invention refers further to compounds of formula (I), (Ia) and (Ib) as described supra, wherein:
R¹ represents a group selected from:

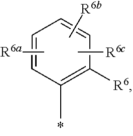

wherein * indicates the point of attachment of said group with the rest of the molecule;
R⁶, R^{6a} and R^{6b} are the same or different and represent, independently from each other, respectively
R⁶ halogen, cyano, C₁-C₄-alkyl, C₁-C₄-haloalkyl, C₁-C₄-alkoxy, C₁-C₄-haloalkoxy, HO—(C₂-C₄-alkoxy)-, (C₁-C₄-alkoxy)-(C₂-C₄-alkoxy)-;
R^{6a} hydrogen, halogen, hydroxy, nitro, cyano, C₁-C₄-alkyl, C₃-C₆-cycloalkyl, C₁-C₄-haloalkyl, C₁-C₄-alkoxy, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—;
R^{6b} hydrogen, halogen, hydroxy, nitro, cyano, C₁-C₄-alkyl, C₃-C₆-cycloalkyl, C₁-C₄-haloalkyl, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—;
R^{6c} represents hydrogen.

According to a further alternative the invention refers to compounds of formula (I), (Ia) and (Ib) as described supra, in which:
R¹ represents a group selected from:

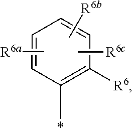

wherein * indicates the point of attachment of said group with the rest of the molecule; and
R⁶, R^{6a} and R^{6b} are the same or different and represent, independently from each other, respectively
R⁶ hydrogen, fluoro, chloro, bromo, cyano, C₁-C₄-alkyl, difluoromethyl, trifluoromethyl, methoxy, ethoxy, difluoromethoxy, trifluoromethoxy, 2-hydroxy-ethoxy, 2-methoxy-ethoxy or F₃C—S—;
R^{6a} hydrogen, fluoro, chloro, bromo, hydroxy, cyano, methyl, difluoromethyl, trifluoromethyl, methoxy, 2-hydroxy-ethoxy, 2-methoxy-ethoxy;
R^{6b} hydrogen, halogen, hydroxy, nitro, cyano, C₁-C₄-alkyl, C₃-C₆-cycloalkyl, C₁-C₄-haloalkyl, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—
R^{6c} represents hydrogen.

In particular the invention refers further to compounds of formula (I), (Ia) and (Ib) as described supra, wherein:
R¹ represents a group selected from:

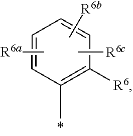

wherein * indicates the point of attachment of said group with the rest of the molecule; and
R⁶, R^{6a} and R^{6b} are the same or different and represent, independently from each other, respectively
R⁶ fluoro, chloro, bromo, cyano, C₁-C₄-alkyl, difluoromethyl, trifluoromethyl, methoxy, ethoxy, difluoromethoxy, trifluoromethoxy or F₃C—S—;
R^{6a} hydrogen, fluoro, chloro, bromo, hydroxy, cyano, methyl, difluoromethyl, trifluoromethyl, methoxy, 2-hydroxy-ethoxy, 2-methoxy-ethoxy;
R^{6b} hydrogen, halogen, hydroxy, nitro, cyano, C₁-C₄-alkyl, C₃-C₆-cycloalkyl, C₁-C₄-haloalkyl, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—
R^{6c} represents hydrogen.

In particular the invention refers further to compounds of formula (I), (Ia) and (Ib) as described supra, wherein:
R¹ represents a group selected from:

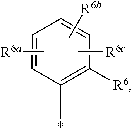

wherein * indicates the point of attachment of said group with the rest of the molecule; and
R⁶ represents hydrogen or halogen and
R^{6a} and R^{6b} adjacent to each other together represent a group selected from —O—CH₂—CH₂— or —O—CH₂—CH₂—O—
R^{6c} represents hydrogen.

In particular the invention refers further to compounds of formula (I), (Ia) and (Ib) as described supra, wherein:
R¹ represents a group selected from:

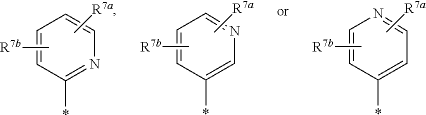

wherein * indicates the point of attachment of said group with the rest of the molecule;
R^{7a} and R^{7b} are the same or different and represent, independently from each other, hydrogen, fluoro, chloro, C₁-C₄-alkyl, difluoromethyl or trifluoromethyl.

According to a further aspect of the present invention compounds of formula (I), (Ia) and (Ib) as described supra are those in which:
R² represents a group selected from:

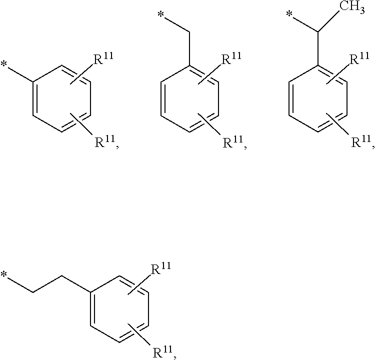

wherein * indicates the point of attachment of said group with the rest of the molecule and in which,
R¹¹ represents independently from each other, hydrogen, halogen, hydroxy, nitro, cyano, C₁-C₄-alkyl, C₂-C₄-alkenyl, C₁-C₄-haloalkyl, C₁-C₄-hydroxyalkyl, C₁-C₄-alkoxy, C₁-C₄-haloalkoxy, HO—(C₂-C₄-alkoxy)-, (C₁-C₄-alkoxy)-(C₂-C₄-alkoxy)-, (C₁-C₄-haloalkyl)-S—, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—.

In a further aspect of the present invention, compounds of formula (I), (Ia) and (Ib) as described supra are those in which:
R² represents a group selected from:
   wherein * indicates the point of attachment of said group with the rest of the molecule and in which,

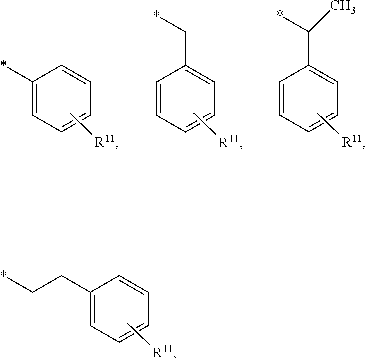

R¹¹ represents independently from each other, halogen, hydroxy, nitro, cyano, C₁-C₄-alkyl, C₂-C₄-alkenyl, C₁-C₄-haloalkyl, C₁-C₄-hydroxyalkyl, C₁-C₄-alkoxy, C₁-C₄-haloalkoxy, HO—(C₂-C₄-alkoxy)-, (C₁-C₄-alkoxy)-(C₂-C₄-alkoxy)-, (C₁-C₄-haloalkyl)-S—, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—.

According to a more particular aspect of the present invention compounds of formula (I), (Ia) and (Ib) as described supra are those in which:
R² represents a group selected from:

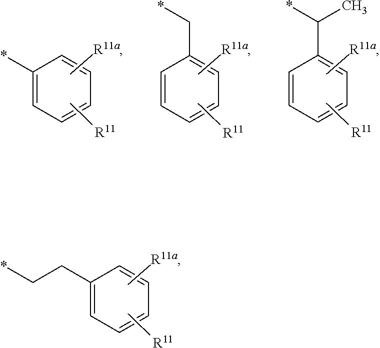

wherein * indicates the point of attachment of said group with the rest of the molecule and in which R¹¹ and R^{11a} are respectively
R¹¹ represents, hydrogen, halogen, hydroxy, nitro, cyano, C₁-C₄-alkyl, C₂-C₄-alkenyl, C₁-C₄-haloalkyl, C₁-C₄-hydroxyalkyl, C₁-C₄-alkoxy, C₁-C₄-haloalkoxy, HO—(C₂-C₄-alkoxy)-, (C₁-C₄-alkoxy)-(C₂-C₄-alkoxy)-, (C₁-C₄-haloalkyl)-S—, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—;
R^{11a} represents a group selected from hydrogen, C₃-C₆-cycloalkyl, morpholino, R^{9a}R^{10a}N—; R^{9a}R^{10a}N—C(O)—; a 5- to 6-membered heteroaryl, which is optionally substituted with methyl or
   represents:

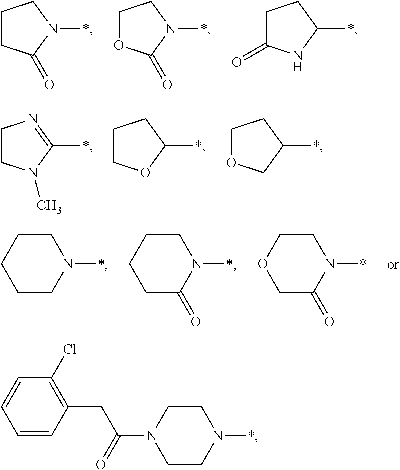

In particular the invention refers further to compounds of formula (I), (Ia) and (Ib) as described supra, wherein:
R² represents a group selected from:

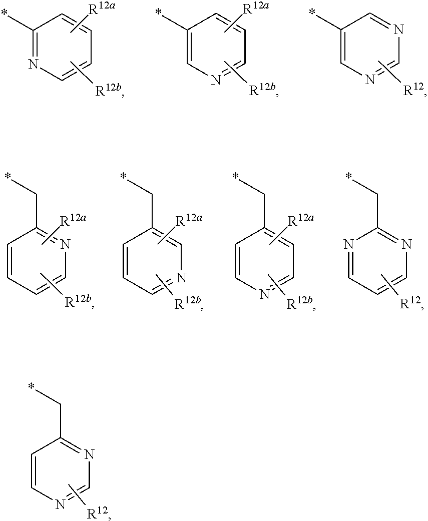

wherein * indicates the point of attachment of said group with the rest of the molecule
R¹² represents hydrogen, halogen, C₁-C₄-alkyl, C₃-C₆-cycloalkyl, methoxy, difluoromethyl or trifluoromethyl;
R^{12a} and R^{12b} represent, independently from each other, hydrogen, halogen, C₁-C₄-alkyl, C₃-C₆-cycloalkyl, methoxy, difluoromethyl or trifluoromethyl.

According to a further aspect of the present invention the compounds of formula (I), (Ia) and (Ib) as described supra, comprise the following groups in which:
R² represents a group selected from:

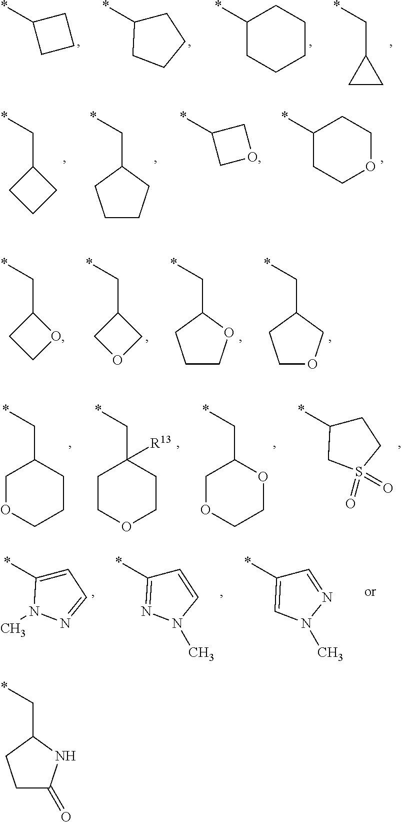

wherein * indicates the point of attachment of said group with the rest of the molecule;
R¹³ represents hydrogen, halogen, cyano or C₁-C₄-alkyl.

More particularly, compounds of formula (I), (Ia) and (Ib) according to the present invention as described supra, have the following groups in which:
R⁵, R^{5a} and R^{5b} are the same or different and represent, independently from each other, hydrogen, halogen, C₁-C₄-alkyl, C₁-C₄-haloalkyl, C₁-C₄-alkoxy or C₁-C₄-haloalkoxy.

In particular the invention refers further to compounds of formula (I), (Ia) and (Ib) as described supra, wherein:

R⁸ represents C₁-C₄-alkyl, C₃-C₆-cycloalkyl or C₁-C₄-haloalkyl.

According to a further aspect of the present invention compounds of formula (I), (Ia) and (Ib) as described supra are those in which:

R⁹ represents, independently from each other, C₁-C₄-alkyl or C₃-C₆-cycloalkyl;
R¹⁰ represents, independently from each other, hydrogen or C₁-C₄-alkyl.

In particular the invention refers further to compounds of formula (I), (Ia) and (Ib) as described supra, wherein:

R^{9a} and R^{10a} together with the nitrogen atom to which they are attached form a 4- to 6-membered nitrogen containing heterocyclic ring, optionally containing one additional heteroatom selected from O, NMe or NH;

In accordance with a further aspect, the invention relates to compounds of formula (Ia) in which:
R¹ represents a group selected from:

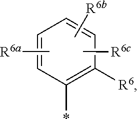

wherein * indicates the point of attachment of said group with the rest of the molecule; and
R⁶, R^{6a} and R^{6b} are the same or different and represent, independently from each other, respectively
   R⁶ hydrogen, fluoro, chloro, bromo, cyano, C₁-C₄-alkyl, difluoromethyl, trifluoromethyl, methoxy, ethoxy, difluoromethoxy, trifluoromethoxy, 2-hydroxy-ethoxy, 2-methoxy-ethoxy or F₃C—S—;
   R^{6a} hydrogen, fluoro, chloro, bromo, hydroxy, cyano, methyl, difluoromethyl, trifluoromethyl, methoxy, 2-hydroxy-ethoxy, 2-methoxy-ethoxy;
   R^{6b} hydrogen, halogen, hydroxy, nitro, cyano, C₁-C₄-alkyl, C₃-C₆-cycloalkyl, C₁-C₄-haloalkyl, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—
   R^{6c} represents hydrogen;
R² represents a group selected from:

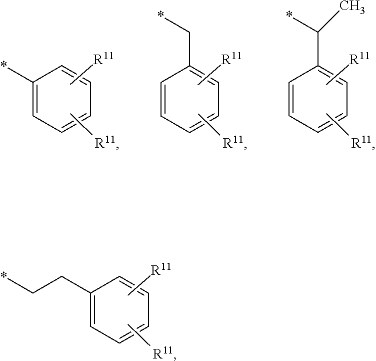

wherein * indicates the point of attachment of said group with the rest of the molecule and in which,
R¹¹ represent independently from each other, hydrogen, halogen, hydroxy, nitro, cyano, C₁-C₄-alkyl, C₂-C₄-alkenyl, C₁-C₄-haloalkyl, C₁-C₄-hydroxyalkyl, C₁-C₄-alkoxy, C₁-C₄-haloalkoxy, HO—(C₂-C₄-alkoxy)-, (C₁-C₄-alkoxy)-(C₂-C₄-alkoxy)-, (C₁-C₄-haloalkyl)-S—, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—.
R³ represents hydrogen or methyl;
R⁴ represents hydrogen
R⁸ represents C₁-C₆-alkyl, C₁-C₄-alkoxy-C₁-C₄-alkyl, C₃-C₆-cycloalkyl or C₁-C₄-haloalkyl;
R⁹ represents, independently from each other, C₁-C₄-alkyl or C₃-C₆-cycloalkyl;
R¹⁰ represents, independently from each other, hydrogen or C₁-C₄-alkyl;

In accordance with a further aspect, the invention relates to compounds of formula (Ia) in which:
R¹ represents a group selected from:

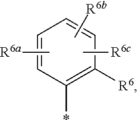

wherein * indicates the point of attachment of said group with the rest of the molecule; and
R⁶, R^{6a} and R^{6b} are the same or different and represent, independently from each other, respectively
   R⁶ hydrogen, fluoro, chloro, bromo, cyano, C₁-C₄-alkyl, difluoromethyl, trifluoromethyl, methoxy, ethoxy, difluoromethoxy, trifluoromethoxy, 2-hydroxy-ethoxy, 2-methoxy-ethoxy or F₃C—S—;
   R^{6a} hydrogen, fluoro, chloro, bromo, hydroxy, cyano, methyl, difluoromethyl, trifluoromethyl, methoxy, 2-hydroxy-ethoxy, 2-methoxy-ethoxy;
   R^{6b} hydrogen, halogen, hydroxy, nitro, cyano, C₁-C₄-alkyl, C₃-C₆-cycloalkyl, C₁-C₄-haloalkyl, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—
   R^{6c} represents hydrogen;
R² represents a group selected from:

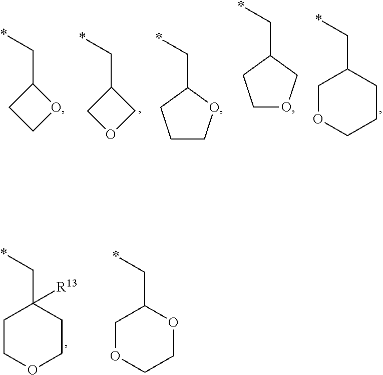

wherein * indicates the point of attachment of said group with the rest of the molecule and in which,
R¹³ represents hydrogen, halogen, cyano or C₁-C₄-alkyl.
R³ represents hydrogen or methyl;
R⁴ represents hydrogen
R⁸ represents C₁-C₆-alkyl, C₁-C₄-alkoxy-C₁-C₄-alkyl, C₃-C₆-cycloalkyl or C₁-C₄-haloalkyl;
R⁹ represents, independently from each other, C₁-C₄-alkyl or C₃-C₆-cycloalkyl;
R¹⁰ represents, independently from each other, hydrogen or C₁-C₄-alkyl;

Furthermore, a particular form of embodiment according to the present invention comprises compounds of formula (Ia) in which:
R¹ represents a group selected from:

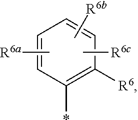

wherein * indicates the point of attachment of said group with the rest of the molecule; and
R⁶, R^{6a} and R^{6b} are the same or different and represent, independently from each other, respectively
   R⁶ hydrogen, fluoro, chloro, bromo, cyano, C₁-C₄-alkyl, difluoromethyl, trifluoromethyl, methoxy, ethoxy, difluoromethoxy, trifluoromethoxy, 2-hydroxy-ethoxy, 2-methoxy-ethoxy or F₃C—S—;
   R^{6a} hydrogen, fluoro, chloro, bromo, hydroxy, cyano, methyl, difluoromethyl, trifluoromethyl, methoxy, 2-hydroxy-ethoxy, 2-methoxy-ethoxy;
   R^{6b} hydrogen, halogen, hydroxy, nitro, cyano, C₁-C₄-alkyl, C₃-C₆-cycloalkyl, C₁-C₄-haloalkyl, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—
   R^{6c} represents hydrogen;
R² represents a group selected from:

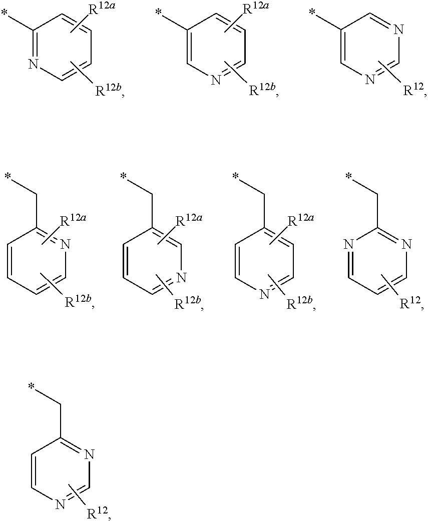

wherein * indicates the point of attachment of said group with the rest of the molecule
R¹² represents hydrogen, halogen, C₁-C₄-alkyl, C₃-C₆-cycloalkyl, methoxy, difluoromethyl or trifluoromethyl;
R^{12a} and R^{12b} represent, independently from each other, hydrogen, halogen, C₁-C₄-alkyl, C₃-C₆-cycloalkyl, methoxy, difluoromethyl or trifluoromethyl;
R³ represents hydrogen or methyl;
R⁴ represents hydrogen
R⁸ represents C₁-C₆-alkyl, C₁-C₄-alkoxy-C₁-C₄-alkyl, C₃-C₆-cycloalkyl or C₁-C₄-haloalkyl;
R⁹ represents, independently from each other, C₁-C₄-alkyl or C₃-C₆-cycloalkyl;
R¹⁰ represents, independently from each other, hydrogen or C₁-C₄-alkyl;
   or an N-oxide, a salt, a hydrate, a solvate, a tautomer or a stereoisomer of said compound, or a salt of said N-oxide, tautomer or stereoisomer.

In particular the invention refers further to compounds of formula (Ia) as described supra, wherein:
R¹ represents a group selected from:

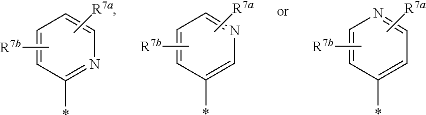

wherein * indicates the point of attachment of said group with the rest of the molecule;
R^{7a} and R^{7b} are the same or different and represent, independently from each other, hydrogen, fluoro, chloro, C₁-C₄-alkyl, difluoromethyl or trifluoromethyl;
R² represents a group selected from:

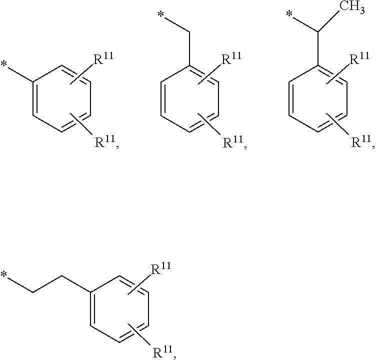

wherein * indicates the point of attachment of said group with the rest of the molecule and in which,
R¹¹ represents, independently from each other, hydrogen, halogen, hydroxy, nitro, cyano, C₁-C₄-alkyl, C₂-C₄-alkenyl, C₁-C₄-haloalkyl, C₁-C₄-hydroxyalkyl, C₁-C₄-alkoxy, C₁-C₄-haloalkoxy, HO—(C₂-C₄-alkoxy)-, (C₁-C₄-alkoxy)-(C₂-C₄-alkoxy)-, (C₁-C₄-haloalkyl)-S—, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—.
R³ represents hydrogen or methyl;
R⁴ represents hydrogen
R⁸ represents C₁-C₆-alkyl, C₁-C₄-alkoxy-C₁-C₄-alkyl, C₃-C₆-cycloalkyl or C₁-C₄-haloalkyl;
R⁹ represents, independently from each other, C₁-C₄-alkyl or C₃-C₆-cycloalkyl;
R¹⁰ represents, independently from each other, hydrogen or C₁-C₄-alkyl;
   or an N-oxide, a salt, a hydrate, a solvate, a tautomer or a stereoisomer of said compound, or a salt of said N-oxide, tautomer or stereoisomer.

In a further aspect of the invention compounds of formula (I) as described above are selected from the group consisting of:

| | |
|---|---|
| 001 | N-[4-(3-chloro-5-cyanophenoxy)-3-sulfamoylphenyl]-2-(2- |
| | chlorophenyl)acetamide |
| 002 | 2-(2-chlorophenyl)-N-4-[3-(dimethylamino)phenoxy]-3- |
| | sulfamoylphenylacetamide |
| 003 | 2-(2-chlorophenyl)-N-4-[(2-chloropyridin-4-yl)oxy]-3- |
| | sulfamoylphenylacetamide |
| 004 | 2-(2-chlorophenyl)-N-[4-(3-isopropylphenoxy)-3- |
| | sulfamoylphenyl]acetamide |
| 005 | 2-(2-chlorophenyl)-N-3-sulfamoyl-4-[3- |
| | (trifluoromethyl)phenoxy]phenylacetamide |
| 006 | 2-(2-chlorophenyl)-N-3-sulfamoyl-4-[3- |
| | (trifluoromethoxy)phenoxy]phenylacetamide |
| 007 | N-[4-(3-acetylphenoxy)-3-sulfamoylphenyl]-2-(2-chlorophenyl)acetamide |
| 008 | N-[4-(1,3-benzodioxol-5-yloxy)-3-sulfamoylphenyl]-2-(2- |
| | chlorophenyl)acetamide |
| 009 | N-[4-(3-acetamidophenoxy)-3-sulfamoylphenyl]-2-(2- |
| | chlorophenyl)acetamide |
| 010 | 2-(2-chlorophenyl)-N-[4-(2-fluorophenoxy)-3-sulfamoylphenyl]acetamide |
| 011 | 2-(2-chlorophenyl)-N-[4-(3-fluorophenoxy)-3-sulfamoylphenyl]acetamide |
| 012 | 2-(2-chlorophenyl)-N-[4-(4-fluorophenoxy)-3-sulfamoylphenyl]acetamide |
| 013 | 2-(2-chlorophenyl)-N-[4-(pyridin-2-yloxy)-3-sulfamoylphenyl]acetamide |
| 014 | 2-(2-chlorophenyl)-N-(4-phenoxy-3-sulfamoylphenyl)acetamide |
| 015 | 2-(2-chlorophenyl)-N-[4-(3-cyanophenoxy)-3-sulfamoylphenyl]acetamide |
| 016 | 2-(2-chlorophenyl)-N-4-[3-(methylsulfonyl)phenoxy]-3- |
| | sulfamoylphenylacetamide |
| 017 | 3-(4-[(2-chlorophenyl)acetyl]amino-2-sulfamoylphenoxy)benzamide |
| 018 | 2-(2-chlorophenyl)-N-[4-(3-methylphenoxy)-3-sulfamoylphenyl]acetamide |
| 019 | 2-(2-chlorophenyl)-N-[4-(pyrimidin-5-yloxy)-3-sulfamoylphenyl]acetamide |
| 020 | 2-(2-chlorophenyl)-N-3-sulfamoyl-4-[3-(4H-1,2,4-triazol-4- |
| | yl)phenoxy]phenylacetamide |
| 021 | 2-(2-chlorophenyl)-N-3-sulfamoyl-4-[3-(1H-tetrazol-5- |
| | yl)phenoxy]phenylacetamide |
| 022 | 2-(2-chlorophenyl)-N-[4-(3-methoxyphenoxy)-3-sulfamoylphenyl]acetamide |
| 023 | 2-(2-chlorophenyl)-N-[4-(4-methoxyphenoxy)-3-sulfamoylphenyl]acetamide |
| 024 | 2-(2-chlorophenyl)-N-4-[3-(difluoromethoxy)phenoxy]-3- |
| | sulfamoylphenylacetamide |
| 025 | 2-(2-chlorophenyl)-N-[4-(3,4-dicyanophenoxy)-3- |
| | sulfamoylphenyl]acetamide |
| 026 | 2-(2-chlorophenyl)-N-4-[3-(morpholin-4-yl)phenoxy]-3- |
| | sulfamoylphenylacetamide |
| 027 | 2-(2-chlorophenyl)-N-[4-(3-4-[(2-chlorophenyl)acetyl]piperazin-1- |
| | ylphenoxy)-3-sulfamoylphenyl]acetamide |
| 028 | 2-(2-chlorophenyl)-N-[4-(pyridin-3-yloxy)-3-sulfamoylphenyl]acetamide |
| 029 | 2-(2-chlorophenyl)-N-4-[(5-chloropyridin-3-yl)oxy]-3- |
| | sulfamoylphenylacetamide |
| 030 | 2-(2-chlorophenyl)-N-[4-(4-cyanophenoxy)-3-sulfamoylphenyl]acetamide |
| 031 | 2-(2-chlorophenyl)-N-4-[3-(difluoromethyl)phenoxy]-3- |
| | sulfamoylphenylacetamide |
| 032 | 2-(2-chlorophenyl)-N-[4-(2-methoxyphenoxy)-3-sulfamoylphenyl]acetamide |
| 033 | 2-(2-chlorophenyl)-N-[4-(3,5-dicyanophenoxy)-3- |
| | sulfamoylphenyl]acetamide |
| 034 | 2-(2-chlorophenyl)-N-[4-(5-cyano-2-methoxyphenoxy)-3- |
| | sulfamoylphenyl]acetamide |
| 035 | 2-(2-chlorophenyl)-N-4-[(2,5-dichloropyridin-3-yl)oxy]-3- |
| | sulfamoylphenylacetamide |
| 036 | 2-(2-chlorophenyl)-N-4-[(5,6-dichloropyridin-3-yl)oxy]-3- |
| | sulfamoylphenylacetamide |
| 037 | 3-(4-[(2-chlorophenyl)acetyl]amino-2-sulfamoylphenoxy)-N- |
| | cyclopropylbenzamide |
| 038 | 2-(2-chlorophenyl)-N-4-[(3-chloropyridin-2-yl)oxy]-3- |
| | sulfamoylphenylacetamide |
| 039 | 2-(2-chlorophenyl)-N-4-[(4-chloropyridin-2-yl)oxy]-3- |
| | sulfamoylphenylacetamide |
| 040 | 2-(2-chlorophenyl)-N-4-[(6-chloropyridin-2-yl)oxy]-3- |
| | sulfamoylphenylacetamide |
| 041 | 2-(2-chlorophenyl)-N-4-[3-(1-methyl-4,5-dihydro-1H-imidazol-2- |
| | yl)phenoxy]-3-sulfamoylphenylacetamide |
| 042 | 2-(2-chlorophenyl)-N-4-[4-(1H-imidazol-1-yl)phenoxy]-3- |
| | sulfamoylphenylacetamide |
| 043 | 2-(2-chlorophenyl)-N-4-[4-(2-oxopyrrolidin-1-yl)phenoxy]-3- |
| | sulfamoylphenylacetamide |
| 044 | 2-(2-chlorophenyl)-N-4-[4-(morpholin-4-yl)phenoxy]-3- |
| | sulfamoylphenylacetamide |
| 045 | 2-(2-chlorophenyl)-N-[4-(5-cyano-2-methylphenoxy)-3- |
| | sulfamoylphenyl]acetamide |
| 046 | 2-(2-chlorophenyl)-N-[4-(3-cyano-2-methylphenoxy)-3- |
| | sulfamoylphenyl]acetamide |
| 047 | 2-(2-chlorophenyl)-N-[4-(3-cyano-4-fluorophenoxy)-3- |
| | sulfamoylphenyl]acetamide |
| 048 | N-4-[(5-chloro-2-cyanopyridin-3-yl)oxy]-3-sulfamoylphenyl-2-(2- |
| | chlorophenyl)acetamide |
| 049 | 2-(2-chlorophenyl)-N-4-[3-(piperidin-1-yl)phenoxy]-3- |
| | sulfamoylphenylacetamide |
| 050 | 2-(2-chlorophenyl)-N-4-[3-(2-oxopyrrolidin-1-yl)phenoxy]-3- |
| | sulfamoylphenylacetamide |
| 051 | 2-(2-chlorophenyl)-N-4-[3-(2-oxo-1,3-oxazolidin-3-yl)phenoxy]-3- |
| | sulfamoylphenylacetamide |
| 052 | 2-(2-chlorophenyl)-N-4-[3-(morpholin-4-ylcarbonyl)phenoxy]-3- |
| | sulfamoylphenylacetamide |
| 053 | 2-(2-chlorophenyl)-N-4-[(4-methyltetrahydro-2H-pyran-4-yl)methoxy]-3- |
| | sulfamoylphenylacetamide |
| 054 | 2-(2-chlorophenyl)-N-4-[(4-fluorotetrahydro-2H-pyran-4-yl)methoxy]-3- |
| | sulfamoylphenylacetamide |
| 055 | 2-(2-chlorophenyl)-N-4-[(4-cyanotetrahydro-2H-pyran-4-yl)methoxy]-3- |
| | sulfamoylphenylacetamide |
| 056 | 2-(2-chlorophenyl)-N-(3-sulfamoyl-4-[2-(trifluoromethyl)pyrimidin-5- |
| | yl]oxyphenyl)acetamide |
| 057 | 2-(2-chlorophenyl)-N-4-[(2-isopropylpyrimidin-5-yl)oxy]-3- |
| | sulfamoylphenylacetamide |
| 058 | 2-(2-chlorophenyl)-N-4-[(2-cyclopropyl-4-methylpyrimidin-5-yl)oxy]-3- |
| | sulfamoylphenylacetamide |
| 059 | N-[4-(3-bromophenoxy)-3-sulfamoylphenyl]-2-(2-chlorophenyl)acetamide |
| 060 | N-[4-(4-bromophenoxy)-3-sulfamoylphenyl]-2-(2-chlorophenyl)acetamide |
| 061 | 2-(2-chlorophenyl)-N-4-[3-(2-methyl-1,3-thiazol-4-yl)phenoxy]-3- |
| | sulfamoylphenylacetamide |
| 062 | 2-(2-chlorophenyl)-N-4-[4-(5-oxopyrrolidin-2-yl)phenoxy]-3- |
| | sulfamoylphenylacetamide |
| 063 | 2-(2-chlorophenyl)-N-4-[4-(2-oxo-1,3-oxazolidin-3-yl)phenoxy]-3- |
| | sulfamoylphenylacetamide |
| 064 | 2-(2-chlorophenyl)-N-3-sulfamoyl-4-[4-(1,3-thiazol-2- |
| | yl)phenoxy]phenylacetamide |
| 065 | N-[4-(2-chlorophenoxy)-3-sulfamoylphenyl]-2-(2-chlorophenyl)acetamide |
| 066 | N-[4-(4-chlorophenoxy)-3-sulfamoylphenyl]-2-(2-chlorophenyl)acetamide |
| 067 | 2-(2-chlorophenyl)-N-4-[3-(piperidin-1-ylcarbonyl)phenoxy]-3- |
| | sulfamoylphenylacetamide |
| 068 | 2-(2-chlorophenyl)-N-3-sulfamoyl-4-[4-(tetrahydrofuran-3- |
| | yl)phenoxy]phenylacetamide |
| 069 | 2-(2-chlorophenyl)-N-[4-(3-cyano-5-fluorophenoxy)-3- |
| | sulfamoylphenyl]acetamide |
| 070 | N-[4-(2-methoxyphenoxy)-3-sulfamoylphenyl]-2-phenylacetamide |
| 071 | N-[4-(2-methoxyphenoxy)-3-sulfamoylphenyl]-2-[4- |
| | (trifluoromethyl)phenyl]acetamide |
| 072 | N-3-sulfamoyl-4-[2-(trifluoromethoxy)phenoxy]phenyl-2-[4- |
| | (trifluoromethyl)phenyl]acetamide |
| 073 | N-[4-(2-chlorophenoxy)-3-sulfamoylphenyl]-2-[4- |
| | (trifluoromethyl)phenyl]acetamide |
| 074 | 2-phenyl-N-3-sulfamoyl-4-[2-(trifluoromethoxy)phenoxy]phenylacetamide |
| 075 | 2-(2-chlorophenyl)-N-4-[(2-oxo-1,2-dihydropyridin-3-yl)oxy]-3- |
| | sulfamoylphenylacetamide |
| 076 | N-[4-(2-chlorophenoxy)-3-sulfamoylphenyl]-2-phenylacetamide |
| 077 | N-[4-(4-chlorophenoxy)-3-sulfamoylphenyl]-2-phenylacetamide |
| 078 | N-4-[(5-chloropyridin-3-yl)oxy]-3-sulfamoylphenyl-2-phenylacetamide |
| 079 | 2-(2-chlorophenyl)-N-4-[(2-chloropyrimidin-5-yl)oxy]-3- |
| | sulfamoylphenylacetamide |
| 080 | 2-(2-chlorophenyl)-N-4-[(5-fluoropyridin-3-yl)oxy]-3- |
| | sulfamoylphenylacetamide |
| 081 | 2-(2-chlorophenyl)-N-4-[(6-chloropyridin-3-yl)oxy]-3- |
| | sulfamoylphenylacetamide |
| 082 | N-[2-chloro-4-(3-chlorophenoxy)-5-sulfamoylphenyl]-2-(2- |
| | chlorophenyl)acetamide |
| 083 | N-[2-chloro-4-(3-chlorophenoxy)-5-sulfamoylphenyl]-2-(2-chloro-3- |
| | fluorophenyl)acetamide |
| 084 | N-[4-(3-chlorophenoxy)-3-sulfamoylphenyl]-2-(3-fluorophenyl)acetamide |
| 085 | N-[4-(3-chlorophenoxy)-3-sulfamoylphenyl]-2-(4-fluorophenyl)acetamide |
| 086 | N-[4-(3-chlorophenoxy)-3-sulfamoylphenyl]-2-[2- |
| | (trifluoromethyl)phenyl]acetamide |
| 087 | N-[4-(3-chlorophenoxy)-3-sulfamoylphenyl]-2-(2- |
| | isopropylphenyl)acetamide |
| 088 | N-[4-(3-chlorophenoxy)-3-sulfamoylphenyl]-2-(2-ethoxyphenyl)acetamide |
| 089 | N-[4-(3-chlorophenoxy)-3-sulfamoylphenyl]-2-[2- |
| | (difluoromethyl)phenyl]acetamide |
| 090 | N-[4-(3-chlorophenoxy)-3-sulfamoylphenyl]-2-2- |
| | [(trifluoromethyl)sulfanyl]phenylacetamide |
| 091 | 2-(2-bromophenyl)-N-[4-(3-chlorophenoxy)-3-sulfamoylphenyl]acetamide |
| 092 | N-[4-(3-chlorophenoxy)-3-sulfamoylphenyl]-2-(4-methylpyridin-3- |
| | yl)acetamide |
| 093 | N-[4-(3-chlorophenoxy)-3-sulfamoylphenyl]-2-(2-chloropyridin-3- |
| | yl)acetamide |
| 094 | N-[4-(3-chlorophenoxy)-3-sulfamoylphenyl]-2-(2-chlorophenyl)-2,2- |
| | difluoroacetamide |
| 095 | 2-(2-chloro-4-methylphenyl)-N-[4-(3-chlorophenoxy)-3- |
| | sulfamoylphenyl]acetamide |
| 096 | 2-(2-chloro-6-methylphenyl)-N-[4-(3-chlorophenoxy)-3- |
| | sulfamoylphenyl]acetamide |
| 097 | 2-(2-chloro-5-methylphenyl)-N-[4-(3-chlorophenoxy)-3- |
| | sulfamoylphenyl]acetamide |
| 098 | 2-(2-chloro-3-fluorophenyl)-N-[4-(3-chlorophenoxy)-3- |
| | sulfamoylphenyl]acetamide |
| 099 | 2-(2-chloro-5-fluorophenyl)-N-[4-(3-chlorophenoxy)-3- |
| | sulfamoylphenyl]acetamide |
| 100 | 2-(2-chloro-6-fluorophenyl)-N-[4-(3-chlorophenoxy)-3- |
| | sutfamoylphenyl]acetamide |
| 101 | 2-(2-chloro-6-methoxyphenyl)-N-[4-(3-chlorophenoxy)-3- |
| | sulfamoylphenyl]acetamide |
| 102 | 2-(2-chloro-5-methoxyphenyl)-N-[4-(3-chlorophenoxy)-3- |
| | sulfamoylphenyl]acetamide |
| 103 | N-[4-(3-chlorophenoxy)-3-sulfamoylphenyl]-2-(2,3- |
| | dichlorophenyl)acetamide |
| 104 | N-[4-(3-chlorophenoxy)-3-sulfamoylphenyl]-2-(2,6- |
| | dichlorophenyl)acetamide |
| 105 | N-[4-(3-chlorophenoxy)-3-sulfamoylphenyl]-2-[2- |
| | (trifluoromethoxy)phenyl]acetamide |
| 106 | N-[4-(3-chlorophenoxy)-3-sulfamoylphenyl]-2,2-difluoro-2-phenylacetamide |
| 107 | N-[4-(3-chlorophenoxy)-3-sulfamoylphenyl]-2-[2-chloro-3- |
| | (trifluoromethyl)phenyl]acetamide |
| 108 | N-[4-(3-chlorophenoxy)-3-sulfamoylphenyl]-2-[2-chloro-6- |
| | (trifluoromethyl)phenyl]acetamide |
| 109 | N-[4-(3-chlorophenoxy)-3-sulfamoylphenyl]-2-[2-chloro-5- |
| | (trifluoromethyl)phenyl]acetamide |
| 110 | N-[4-(3-chlorophenoxy)-3-sulfamoylphenyl]-2-(2,4- |
| | dichlorophenyl)acetamide |
| 111 | N-[4-(3-chlorophenoxy)-3-sulfamoylphenyl]-2-(4,6-dichloropyridin-3- |
| | yl)acetamide |
| 112 | N-[4-(3-chlorophenoxy)-3-sulfamoylphenyl]-2-(3-chloropyridin-2- |
| | yl)acetamide |
| 113 | N-[4-(3-chlorophenoxy)-3-sulfamoylphenyl]-2-[2- |
| | (difluoromethoxy)phenyl]acetamide |
| 114 | N-[4-(3-chlorophenoxy)-3-sulfamoylphenyl]-2-(2,5- |
| | dichlorophenyl)acetamide |
| 115 | 2-[6-chloro-2,3-difluoro-4-(trifluoromethyl)phenyl]-N-[4-(3-chlorophenoxy)- |
| | 3-sulfamoylphenyl]acetamide |
| 116 | N-[4-(3-chlorophenoxy)-3-sulfamoylphenyl]-2-[4- |
| | (trifluoromethyl)phenyl]acetamide |
| 117 | 2-(5-bromo-2-chlorophenyl)-N-[4-(3-chlorophenoxy)-3- |
| | sulfamoylphenyl]acetamide |
| 118 | 2-(4-bromo-2-chloro-5-methylphenyl)-N-[4-(3-chlorophenoxy)-3- |
| | sulfamoylphenyl]acetamide |
| 119 | N-[4-(3-chlorophenoxy)-3-sulfamoylphenyl]-2-(3-chloropyridin-4- |
| | yl)acetamide |
| 120 | 2-(2-chloro-6-fluoro-3-methylphenyl)-N-[4-(3-chlorophenoxy)-3- |
| | sulfamoylphenyl]acetamide |
| 121 | 2-(6-chloro-2-fluoro-3-methylphenyl)-N-[4-(3-chlorophenoxy)-3- |
| | sulfamoylphenyl]acetamide |
| 122 | 2-(2-chloro-3,6-difluorophenyl)-N-[4-(3-chlorophenoxy)-3- |
| | sulfamoylphenyl]acetamide |
| 123 | 2-(2-chloro-4,5-difluorophenyl)-N-[4-(3-chlorophenoxy)-3- |
| | sulfamoylphenyl]acetamide |
| 124 | N-[4-(3-chlorophenoxy)-3-sulfamoylphenyl]-2-(2,3-dichloro-6- |
| | fluorophenyl)acetamide |
| 125 | N-[4-(3-chlorophenoxy)-3-sulfamoylphenyl]-2-(2,3,6- |
| | trichlorophenyl)acetamide |
| 126 | N-[4-(3-chlorophenoxy)-3-sulfamoylphenyl]-2-(2,6-dichloro-4- |
| | methylphenyl)acetamide |
| 127 | N-[4-(3-chlorophenoxy)-3-sulfamoylphenyl]-2-[2,3-dichloro-6- |
| | (trifluoromethyl)phenyl]acetamide |
| 128 | N-[4-(3-chlorophenoxy)-3-sulfamoylphenyl]-2-(2,6-dichloro-3- |
| | methylphenyl)acetamide |
| 129 | N-[4-(3-chlorophenoxy)-3-sulfamoylphenyl]-2-(2,6-dichloro-3- |
| | cyclopropylphenyl)acetamide |
| 130 | N-[4-(3-chlorophenoxy)-3-sulfamoylphenyl]-2-[2,6-dichloro-3- |
| | (trifluoromethyl)phenyl]acetamide |
| 131 | 2-(3-bromo-2,6-dichlorophenyl)-N-[4-(3-chlorophenoxy)-3- |
| | sulfamoylphenyl]acetamide |
| 132 | 2-(3-bromo-2-chloro-6-methylphenyl)-N-[4-(3-chlorophenoxy)-3- |
| | sulfamoylphenyl]acetamide |
| 133 | 2-(3-bromo-6-chloro-2-methylphenyl)-N-[4-(3-chlorophenoxy)-3- |
| | sulfamoylphenyl]acetamide |
| 134 | N-[4-(3-chlorophenoxy)-3-sulfamoylphenyl]-2-[2-chloro-5-(1,1,2,2- |
| | tetrafluoroethoxy)phenyl]acetamide |
| 135 | N-[4-(3-chlorophenoxy)-3-sulfamoylphenyl]-2-[2-chloro-4- |
| | (trifluoromethyl)phenyl]acetamide |
| 136 | 2-(2-chlorophenyl)-N-(4-[3-(methylsulfonyl)benzyl]oxy-3- |
| | sulfamoylphenyl)acetamide |
| 137 | 2-(2-chlorophenyl)-N-4-[(2-fluorobenzyl)oxy]-3-sulfamoylphenylacetamide |
| 138 | 2-(2-chlorophenyl)-N-4-[(4-cyanobenzyl)oxy]-3-sulfamoylphenylacetamide |
| 139 | N-4-[(3-chlorobenzyl)oxy]-3-sulfamoylphenyl-2-(2-chlorophenyl)acetamide |
| 140 | 2-(2-chlorophenyl)-N-4-[(3-methoxybenzyl)oxy]-3- |
| | sulfamoylphenylacetamide |
| 141 | N-[4-(benzyloxy)-3-sulfamoylphenyl]-2-(2-chlorophenyl)acetamide |
| 142 | 2-(2-chlorophenyl)-N-4-[(3-cyanobenzyl)oxy]-3-sulfamoylphenylacetamide |
| 143 | 2-(2-chlorophenyl)-N-4-[(4-fluorobenzyl)oxy]-3-sulfamoylphenylacetamide |
| 144 | N-4-[(2-chlorobenzyl)oxy]-3-sulfamoylphenyl-2-(2-chlorophenyl)acetamide |
| 145 | 2-(2-chlorophenyl)-N-4-[(2-cyanobenzyl)oxy]-3-sulfamoylphenylacetamide |
| 146 | N-[4-(benzyloxy)-3-sulfamoylphenyl]-2-phenylacetamide |
| 147 | 2-(2-chlorophenyl)-N-(4-[4-(methylsulfonyl)benzyl]oxy-3- |
| | sulfamoylphenyl)acetamide |
| 148A | 2-(2-chlorophenyl)-N-[4-(1-phenylethoxy)-3-sulfamoylphenyl]acetamide |
| 148B | 2-(2-chlorophenyl)-N-[4-(1-phenylethoxy)-3-sulfamoylphenyl]acetamide |
| 149 | 2-(2-chlorophenyl)-N-[4-(pyridin-3-ylmethoxy)-3- |
| | sulfamoylphenyl]acetamide |
| 150 | 2-(2-chlorophenyl)-N-[4-(pyridin-2-ylmethoxy)-3- |
| | sulfamoylphenyl]acetamide |
| 151 | 2-(2-chlorophenyl)-N-[4-(pyridin-4-ylmeihoxy)-3- |
| | sulfamoylphenyl]acetamide |
| 152 | N-[4-(pyridin-2-ylmethoxy)-3-sulfamoylphenyl]-2-[4- |
| | (trifluoromethyl)phenyl]acetamide |
| 153 | 2-(2-chlorophenyl)-N-[4-(pyrimidin-4-ylmethoxy)-3- |
| | sulfamoylphenyl]acetamide |
| 154 | 2-(2-chlorophenyl)-N-[4-(pyrimidin-2-ylmethoxy)-3- |
| | sulfamoylphenyl]acetamide |
| 155 | 2-(2-chlorophenyl)-N-[4-(2-phenylethoxy)-3-sulfamoylphenyl]acetamide |
| 156 | 2-(2-chlorophenyl)-N-4-[2-(3-chlorophenyl)ethoxy]-3- |
| | sulfamoylphenylacetamide |
| 157 | 2-(2-chlorophenyl)-N-[4-(cyclobutylmethoxy)-3-sulfamoylphenyl]acetamide |
| 158 | 2-(2-chlorophenyl)-N-[4-(oxetan-2-ylmethoxy)-3- |
| | sulfamoylphenyl]acetamide |
| 159 | 2-(2-chlorophenyl)-N-[4-(oxetan-3-ylmethoxy)-3- |
| | sulfamoylphenyl]acetamide |
| 160 | 2-(2-chlorophenyl)-N-[4-(cyclopentylmethoxy)-3- |
| | sulfamoylphenyl]acetamide |
| 161 | 2-(2-chlorophenyl)-N-[3-sulfamoyl-4-(tetrahydrofuran-2- |
| | ylmethoxy)phenyl]acetamide |
| 162 | 2-(2-chlorophenyl)-N-[3-sulfamoyl-4-(tetrahydrofuran-3- |
| | ylmethoxy)phenyl]acetamide |
| 163 | 2-(2-chloro-5-fluorophenyl)-N-[3-sulfamoyl-4-(tetrahydro-2H-pyran-4- |
| | ylmethoxy)phenyl]acetamide |
| 164 | 2-(2-chlorophenyl)-N-[3-sulfamoyl-4-(tetrahydro-2H-pyran-4- |
| | ylmethoxy)phenyl]acetamide |
| 165 | 2-(2-chlorophenyl)-N-[3-sulfamoyl-4-(tetrahydro-2H-pyran-3- |
| | ylmethoxy)phenyl]acetamide |
| 166 | 2-(2-chloro-6-fluorophenyl)-N-[3-sulfamoyl-4-(tetrahydro-2H-pyran-4- |
| | ylmethoxy)phenyl]acetamide |
| 167 | 2-(2-chloro-3-fluorophenyl)-N-[3-sulfamoyl-4-(tetrahydro-2H-pyran-4- |
| | ylmethoxy)phenyl]acetamide |
| 168 | 2-(2-chlorophenyl)-N-5-sulfamoyl-6-[3-(trifluoromethyl)phenoxy]pyridin-3- |
| | ylacetamide |
| 169 | 2-phenyl-N-5-sulfamoyl-6-[3-(trifluoromethyl)phenoxy]pyridin-3- |
| | ylacetamide |
| 170 | N-[4-(3-chlorophenoxy)-3-fluoro-5-sulfamoylphenyl]-2-phenylacetamide |
| 171 | N-[4-(3-chlorophenoxy)-3-fluoro-5-sulfamoylphenyl]-2-(2- |
| | methylphenyl)acetamide |
| 172 | N-[4-(3-chlorophenoxy)-3-fluoro-5-sulfamoylphenyl]-2-(3- |
| | methylphenyl)acetamide |
| 173 | 2-(2-chlorophenyl)-N-4-[3-(3-oxomorpholin-4-yl)phenoxy]-3- |
| | sulfamoylphenylacetamide |
| 174 | 2-(2-chlorophenyl)-N-4-[4-(3-oxomorpholin-4-yl)phenoxy]-3- |
| | sulfamoylphenylacetamide |
| 175 | 2-(2-chlorophenyl)-N-4-[4-(2-oxopiperidin-1-yl)phenoxy]-3- |
| | sulfamoylphenylacetamide |
| 176 | 2-(2-chlorophenyl)-N-4-[3-(2-oxopiperidin-1-yl)phenoxy]-3- |
| | sulfamoylphenylacetamide |
| 177 | 2-(2-chlorophenyl)-N-4-[3-(prop-1-en-2-yl)phenoxy]-3- |
| | sulfamoylphenylacetamide |
| 178 | 2-(2-chlorophenyl)-N-4-[2-(prop-1-en-2-yl)phenoxy]-3- |
| | sulfamoylphenylacetamide |
| 179 | N-[4-(3-chlorophenoxy)-3-sulfamoylphenyl]-2-(4-methylphenyl)acetamide |
| 180 | N-[4-(3-chlorophenoxy)-3-sulfamoylphenyl]-2-(4-chlorophenyl)acetamide |
| 181 | N-[4-(3-chlorophenoxy)-3-sulfamoylphenyl]-2-(pyridin-3-yl)acetamide |
| 182 | N-[4-(3-chlorophenoxy)-3-sulfamoylphenyl]-2-(2-methylphenyl)acetamide |
| 183 | N-[4-(3-chlorophenoxy)-3-sulfamoylphenyl]-2-(3-methylphenyl)acetamide |
| 184 | N-[4-(3-chlorophenoxy)-3-sulfamoylphenyl]-2-phenylpropanamide |
| 185 | N-[4-(3-chlorophenoxy)-3-sulfamoylphenyl]-2-(pyridin-2-yl)acetamide |
| 186 | N-[4-(3-chlorophenoxy)-3-sulfamoylphenyl]-2-(3-chlorophenyl)acetamide |
| 187 | N-[4-(3-chlorophenoxy)-3-sulfamoylphenyl]-2-(2-chlorophenyl)acetamide |
| 188 | N-[4-(3-chlorophenoxy)-3-sulfamoylphenyl]-2-(pyridin-4-yl)acetamide |
| 189 | N-[4-(3-chlorophenoxy)-3-sulfamoylphenyl]-2-(6-methylpyridin-2- |
| | yl)acetamide |
| 190 | N-[4-(3-chlorophenoxy)-3-sulfamoylphenyl]-2-(4-methoxyphenyl)acetamide |
| 191 | N-[4-(3-chlorophenoxy)-3-sulfamoylphenyl]-2-(3-methoxyphenyl)acetamide |
| 192 | N-[4-(3-chlorophenoxy)-3-sulfamoylphenyl]-2-(2-methoxyphenyl)acetamide |
| 193 | N-[4-(3-chlorophenoxy)-3-sulfamoylphenyl]-2-(5-methylpyridin-2- |
| | yl)acetamide |
| 194 | (2S)-N-[4-(3-chlorophenoxy)-3-sulfamoylphenyl]-2-phenylpropanamide |
| 195 | (2R)-N-[4-(3-chlorophenoxy)-3-sulfamoylphenyl]-2-phenylpropanamide |
| 196 | N-[4-(3-chlorophenoxy)-3-sulfamoylphenyl]-2-(2- |
| | chlorophenyl)propanamide |
| 197 | 2-(2-[4-(3-chlorophenoxy)-3-sulfamoylphenyl]amino-2-oxoethyl)-N-(2- |
| | methoxyethyl)-N-methylbenzamide |
| 198 | 2-(2-[4-(3-chlorophenoxy)-3-sulfamoylphenyl]amino-2-oxoethyl)-N,N- |
| | dimethylbenzamide |
| 199 | N-[4-(cyclohexyloxy)-3-sulfamoylphenyl]-2-phenylacetamide |
| 200 | 2-(2-chlorophenyl)-N-[4-(cyclohexyloxy)-3-sulfamoylphenyl]acetamide |
| 201 | 3-(2-[4-(3-chlorophenoxy)-3-sulfamoylphenyl]amino-2-oxoethyl)-N-(2- |
| | methoxyethyl)benzamide |
| 202 | 3-(2-[4-(3-chlorophenoxy)-3-sulfamoylphenyl]amino-2-oxoethyl)-N,N- |
| | dimethylbenzamide |
| 203 | 3-(2-[4-(3-chlorophenoxy)-3-sulfamoylphenyl]amino-2-oxoethyl)-N- |
| | methylbenzamide |
| 204 | N-[4-(cyclobutyloxy)-3-sulfamoylphenyl]-2-phenylacetamide |
| 205 | 2-(2-chlorophenyl)-N-[4-(cyclobutyloxy)-3-sulfamoylphenyl]acetamide |
| 206 | 2-phenyl-N-[3-sulfamoyl-4-(tetrahydro-2H-pyran-4-yloxy)phenyl]acetamide |
| 207 | 2-(2-chlorophenyl)-N-[3-sulfamoyl-4-(tetrahydro-2H-pyran-4- |
| | yloxy)phenyl]acetamide |
| 208 | 3-(2-[4-(3-chlorophenoxy)-3-sulfamoylphenyl]amino-2-oxoethyl)-N-(2- |
| | methoxyethyl)-N-methylbenzamide |
| 209 | N-[4-(3-chlorophenoxy)-3-sulfamoylphenyl]-2-(5-chloropyridin-2- |
| | yl)acetamide |
| 210 | N-[4-(3-chlorophenoxy)-3-sulfamoylphenyl]-2-[3-(2- |
| | methoxyethoxy)phenyl]acetamide |
| 211 | N-[4-(3-chlorophenoxy)-3-sulfamoylphenyl]-2-[2-(2- |
| | methoxyethoxy)phenyl]acetamide |
| 212 | N-[4-(3-chlorophenoxy)-3-sulfamoylphenyl]-2-[3-(2- |
| | hydroxyethoxy)phenyl]acetamide |
| 213 | N-[4-(3-chlorophenoxy)-3-sulfamoylphenyl]-2-[2-(2- |
| | hydroxyethoxy)phenyl]acetamide |
| 214 | N-[4-(3-chlorophenoxy)-3-sulfamoylphenyl]-2-(2-fluorophenyl)acetamide |
| 215 | N-[4-(oxetan-3-yloxy)-3-sulfamoylphenyl]-2-phenylacetamide |
| 216 | 2-(2-chlorophenyl)-N-[4-(oxetan-3-yloxy)-3-sulfamoylphenyl]acetamide |
| 217 | N-[4-(cyclopentyloxy)-3-sulfamoylphenyl]-2-phenylacetamide |
| 218 | 2-(2-chlorophenyl)-N-[4-(cyclopentyloxy)-3-sulfamoylphenyl]acetamide |
| 219 | N-4-[(1-methylpiperidin-3-yl)oxy]-3-sulfamoylphenyl-2-phenylacetamide |
| 220 | 2-(2-chlorophenyl)-N-4-[(1-methylpiperidin-3-yl)oxy]-3- |
| | sulfamoylphenylacetamide |
| 221 | N-4-[(1-methylpyrrolidin-3-yl)oxy]-3-sulfamoylphenyl-2-phenylacetamide |
| 222 | 2-(2-chlorophenyl)-N-4-[(1-methylpyrrolidin-3-yl)oxy]-3- |
| | sulfamoylphenylacetamide |
| 223 | N-[4-(4-chlorophenoxy)-3-sulfamoylphenyl]-2-(4-fluorophenyl)acetamide |
| 224 | N-[4-(3-chlorophenoxy)-3-sulfamoylphenyl]-2-(4-cyanophenyl)acetamide |
| 225 | N-[4-(3-chlorophenoxy)-3-sulfamoylphenyl]-2-(2-cyanophenyl)acetamide |
| 226 | N-[4-(3-chlorophenoxy)-3-sulfamoylphenyl]-2-(3-cyanophenyl)acetamide |
| 227 | N-[4-(4-chlorophenoxy)-3-sulfamoylphenyl]-2-[4- |
| | (trifluoromethyl)phenyl]acetamide |
| 228 | N-[4-(4-chlorophenoxy)-3-sulfamoylphenyl]-2-(4-chlorophenyl)acetamide |
| 229 | N-[4-(4-chlorophenoxy)-3-sulfamoylphenyl]-2-(4-methoxyphenyl)acetamide |
| 230 | N-[4-(4-chlorophenoxy)-3-sulfamoylphenyl]-2-(2-fluorophenyl)acetamide |
| 231 | 2-(2-chloro-4-fluorophenyl)-N-[4-(4-chlorophenoxy)-3- |
| | sulfamoylphenyl]acetamide |
| 232 | 2-(2-chlorophenyl)-N-4-[(1,1-dioxidotetrahydrothiophen-3-yl)oxy]-3- |
| | sulfamoylphenylacetamide |
| 233 | 2-(2-chlorophenyl)-N-4-[(1-methyl-1H-pyrazol-4-yl)oxy]-3- |
| | sulfamoylphenylacetamide |
| 234 | N-[4-(4-chlorophenoxy)-3-sulfamoylphenyl]-2-[4- |
| | (difluoromethyl)phenyl]acetamide |
| 235 | 2-(2-chloro-4-methoxyphenyl)-N-[4-(4-chlorophenoxy)-3- |
| | sulfamoylphenyl]acetamide |
| 236 | 2-(2-chlorophenyl)-N-4-[(1-methyl-1H-pyrazol-3-yl)oxy]-3- |
| | sulfamoylphenylacetamide |
| 237 | 2-(2-chlorophenyl)-N-4-[(1-methyl-1H-pyrazol-5-yl)oxy]-3- |
| | sulfamoylphenylacetamide |
| 238 | 2-(2-chlorophenyl)-N-4-[(1-methylpiperidin-4-yl)oxy]-3- |
| | sulfamoylphenylacetamide |
| 239 | 2-(2-chlorophenyl)-N-(4-[5-methyl-2-(pyridin-3-yl)-1,3-thiazol-4-yl]oxy-3- |
| | sulfamoylphenyl)acetamide |
| 240 | N-[4-(3-chlorophenoxy)-2-methyl-5-sulfamoylphenyl]-2-(2- |
| | chlorophenyl)acetamide |
| 241 | 2-(2-chlorophenyl)-N-{4-[(1-oxidotetrahydrothiophen-3-yl)oxy]-3- |
| | sulfamoylphenyl}acetamide |
| 242 | N-[4-(4-chlorophenoxy)-3-sulfamoylphenyl]-2-[2,6-dichloro-4- |
| | (trifluoromethyl)phenyl]acetamide |
| 243 | N-[4-(4-chlorophenoxy)-3-sulfamoylphenyl]-2-(2,5-dichloro-4- |
| | cyanophenyl)acetamide |
| 244 | N-[4-(3-chlorophenoxy)-3-sulfamoylphenyl]-2-phenylacetamide |
| 245 | N-[4-(cyclopropylmethoxy)-3-sulfamoylphenyl]-2-phenylacetamide |
| 246 | N-[4-(3,5-dimethylphenoxy)-3-sulfamoylphenyl]-2-phenylacetamide |
| 247 | N-[4-(2,4-difiuorophenoxy)-3-sulfamoylphenyl]-2-phenylacetamide |
| 248 | N-[4-(4-fluorophenoxy)-3-sulfamoylphenyl]-2-phenylacetamide |
| 249 | N-[4-(3-fluorophenoxy)-3-sulfamoylphenyl]-2-phenylacetamide |
| 250 | N-[4-(3-methoxyphenoxy)-3-sulfamoylphenyl]-2-phenylacetamide |
| 251 | N-[4-(2-fluoro-5-methylphenoxy)-3-sulfamoylphenyl]-2-phenylacetamide |
| 252 | 2-phenyl-N-3-sulfamoyl-4-[4-(trifluoromethoxy)phenoxy]phenylacetamide |
| 253 | 2-phenyl-N-3-sulfamoyl-4-[3-(trifluoromethyl)phenoxy]phenylacetamide |
| 254 | N-[4-(3,5-dimethoxyphenoxy)-3-sulfamoylphenyl]-2-phenylacetamide |
| 255 | N-[4-(3-cyanophenoxy)-3-sulfamoylphenyl]-2-phenylacetamide |
| 256 | N-[4-(3-chlorophenoxy)-3-sulfamoylphenyl]-2-(4-hydroxyphenyl)acetamide |
| 257 | 2-(2-chloro-6-methoxy-4-methylphenyl)-N-[4-(3-chlorophenoxy)-3- |
| | sulfamoylphenyl]acetamide |
| 258 | 2-(2-chloro-6-fluorophenyl)-N-[4-(3-chlorophenoxy)-3- |
| | sulfamoylphenyl]propanamide |
| 259 | 2-(2-chloro-4,6-difluorophenyl)-N-[4-(3-chlorophenoxy)-3- |
| | sulfamoylphenyl]acetamide |
| 260 | N-[4-(3-chlorophenoxy)-3-sulfamoylphenyl]-2-(2,6- |
| | dichlorophenyl)propanamide |
| 261 | 2-(2-chlorophenyl)-N-4-[(²H₅)phenyloxy]-3-sulfamoylphenylacetamide |
| 262 | 2-(2-chlorophenyl)-N-(4-{[4-chloro(²H₄)phenyl]oxy}-3- |
| | sulfamoylphenyl)acetamide |
| 263 | 2-(2-chlorophenyl)-N-(4-{[2-chloro(²H₄)phenyl]oxy}-3- |
| | sulfamoylphenyl)acetamide |
| 264 | 2-(2-chlorophenyl)-N-4-[4-(2-hydroxypropan-2-yl)phenoxy]-3- |
| | sulfamoylphenylacetamide |
| 265 | 2-(2-chlorophenyl)-N-4-[(2,2-dimethyltetrahydro-2H-pyran-4-yl)methoxy]-3- |
| | sulfamoylphenylacetamide |
| 266 | 2-(2-chlorophenyl)-N-{4-[(1R,5S,6r)-3-oxabicyclo[3.1.0]hex-6-ylmethoxy]- |
| | 3-sulfamoylphenyl}acetamide |
| 267 | 2-(2-chlorophenyl)-N-4-[(4-chlorotetrahydro-2H-pyran-4-yl)methoxy]-3- |
| | sulfamoylphenylacetamide |
| 268 | 2-(2-chlorophenyl)-N-[4-(1,4-dioxan-2-ylmethoxy)-3- |
| | sulfamoylphenyl]acetamide |
| 269 | 2-(2-chlorophenyl)-N-3-sulfamoyl-4-[(2,2,6,6-tetramethyltetrahydro-2H- |
| | pyran-4-yl)oxy]phenylacetamide |
| 270 | N-[4-(3-chlorophenoxy)-3-methyl-5-sulfamoylphenyl]-2-(2- |
| | chlorophenyl)acetamide |
| 271 | N-[4-(3-chlorophenoxy)-3-methyl-5-sulfamoylphenyl]-2-phenylacetamide |
| 272 | methyl 2-(4-[(2-chlorophenyl)acetyl]amino-2-sulfamoylphenoxy)benzoate |
| 273 | methyl 4-(4-[(2-chlorophenyl)acetyl]amino-2-sulfamoylphenoxy)benzoate |
| 274 | 2-(2-chlorophenyl)-N-4-[3-(2-hydroxypropan-2-yl)phenoxy]-3- |
| | sulfamoylphenylacetamide |
| 275 | 2-(2-chlorophenyl)-N-4-[2-(2-hydroxypropan-2-yl)phenoxy]-3- |
| | sulfamoylphenylacetamide |
| 276 | N-[4-(4-chlorophenoxy)-3-sulfamoylphenyl]-2-(2,3-dihydro-1,4- |
| | benzodioxin-6-yl)acetamide |
| 277 | 2-(7-chloro-2,3-dihydro-1,4-benzodioxin-6-yl)-N-[4-(3-chlorophenoxy)-3- |
| | sulfamoylphenyl]acetamide |
| 278 | 2-(5-chloro-2,3-dihydro-1-benzofuran-4-yl)-N-[4-(3-chlorophenoxy)-3- |
| | sulfamoylphenyl]acetamide |
| 279 | 2-(2-fluorophenyl)-N-[3-sulfamoyl-4-(tetrahydro-2H-pyran-4- |
| | ylmethoxy)phenyl]acetamide |
| 280 | N-[3-sulfamoyl-4-(tetrahydro-2H-pyran-4-ylmethoxy)phenyl]-2-[2- |
| | (trifluoromethyl)phenyl]acetamide |
| 281 | 2-[2-(difluoromethyl)phenyl]-N-[3-sulfamoyl-4-(tetrahydro-2H-pyran-4- |
| | ylmethoxy)phenyl]acetamide |
| 282 | 2-(2-chloro-4-fluorophenyl)-N-[3-sulfamoyl-4-(tetrahydro-2H-pyran-4- |
| | ylmethoxy)phenyl]acetamide |
| 283 | 2-(2-Chlorophenyl)-N-(3-sulfamoyl-4-{[6-(trifluoromethyl)pyridin-3- |
| | yl]oxy}¬phenyl)-acetamide |
| 284 | 2-(2-Chlorophenyl)-N-(4-{[5-chloro-4-(trifluoromethyl)pyridin-2-yl]oxy}-3- |
| | sulfamoyl-phenyl)acetamide |
| 285 | N-[4-(3-chlorophenoxy)-3-sulfamoylphenyl]-2-phenyl(2H2)acetamide |
| 286 | N-{4-[(6-Chloro-5-fluoropyridin-3-yl)oxy]-3-sulfamoylphenyl}-2-(2- |
| | chlorophenyl)-acetamide |
| 287 | 2-(2-Chlorophenyl)-N-{4-[(4,4-difluoro-1-hydroxycyclohexyl)methoxy]-3- |
| | sulfamoyl-phenyl}acetamide |
| 288 | 2-(2-Chlorophenyl)-N-{4-[(1-hydroxycyclohexyl)methoxy]-3- |
| | sulfamoylphenyl}-acetamide |
| 289 | N-[4-(3-Chlorophenoxy)-3-fluoro-5-sulfamoylphenyl]-2-(2- |
| | fluorophenyl)acetamide |
| 290 | N-[4-(3-chlorophenoxy)-3-fluoro-5-sulfamoylphenyl]-2-[2-(difluoromethyl)- |
| | phenyl]acetamide |
| 291 | N-[4-(3-chlorophenoxy)-3-fluoro-5-sulfamoylphenyl]-2-(2- |
| | chlorophenyl)acetamide |
| 292 | 2-(2-chloro-5-fluorophenyl)-N-[4-(3-chlorophenoxy)-3-fluoro-5-sulfamoyl- |
| | phenyl]acetamide |
| 293 | N-[6-(3-Chlorophenoxy)-5-sulfamoylpyridin-3-yl]-2-(2- |
| | fluorophenyl)acetamide |
| 294 | N-[6-(3-Chlorophenoxy)-5-sulfamoylpyridin-3-yl]-2-[2- |
| | (trifluoromethyl)phenyl]-acetamide |
| 295 | N-[6-(3-Chlorophenoxy)-5-sulfamoylpyridin-3-yl]-2-[2- |
| | (difluoromethyl)phenyl]-acetamide |
| 296 | 2-(2-Chloro-5-fluorophenyl)-N-[6-(3-chlorophenoxy)-5-sulfamoylpyridin-3- |
| | yl]-acetamide |

or an N-oxide, a salt, a hydrate, a solvate, a tautomer or a stereoisomer of said compound, or a salt of said N-oxide, tautomer or stereoisomer.

One aspect of the invention are compounds of formula (I), (Ia), (Ib) as described in the examples, as characterized by their names in the title and their structures as well as the subcombinations of all residues specifically disclosed in the compounds of the examples.

Another aspect of the present invention are intermediates according to formula 9

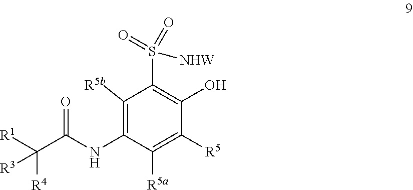

whereby R¹, R³, R⁴, R⁵, R^{5a} and R^{5b} are defined according to the description and claims and W corresponds to either a hydrogen atom or a protecting group (e.g., N-(dimethylamino)methylene or 2,4-dimethoxybenzyl). The intermediates according to formula 9 are used for the synthesis of the compounds of formula (I), more in particular of compounds of formula 6, and compounds of formula (Ia).

Furthermore the present invention refers to intermediates according to formula 13 or 14

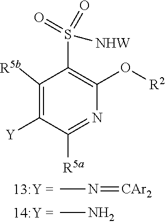

whereby R², R^{5a} and R^{5b} are defined according to the description and claims, Ar stands for aryl and W corresponds to either a hydrogen atom or a protecting group (e.g., N-(dimethylamino)methylene or 2,4-dimethoxybenzyl). The intermediates according to formula 13 or 14 are used for the synthesis of the compounds of formula (I), more in particular of compounds of formula 15, and compounds of formula (Ib).

Specific intermediates for the synthesis of compounds of formula (I) according to present invention are:

| | |
|---|---|
| 002 | N-(2,4-Dimethoxybenzyl)-2-fluoro-5-nitrobenzenesulfonamide |
| 003 | 2,4-Dichloro-N-(2,4-dimethoxybenzyl)-5-nitrobenzenesulfonamide |
| 004 | N-(2,4-Dimethoxybenzyl)-2,3-difluoro-5-nitrobenzenesulfonamide |
| 008 | 2-(2-Chlorophenyl)-N-(4-hydroxy-3-sulfamoylphenyl)acetamide |
| 009 | 2-(2-Chloro-3-fluorophenyl)-N-(4-hydroxy-3- |
| | sulfamoylphenyl)acetamide |
| 010 | 2-(2-Chloro-6-fluorophenyl)-N-(4-hydroxy-3- |
| | sulfamoylphenyl)acetamide |
| 011 | 5-Bromo-2-hydroxypyridine-3-sulfonamide |
| 013 | 5-Amino-2-[3-(trifluoromethyl)phenoxy]pyridine-3-sulfonamide |
| 057 | N-(2,4-Dimethoxybenzyl)-2-fluoro-4-methyl-5- |
| | nitrobenzenesulfonamide |
| 068 | N-(2,4-Dimethoxybenzyl)-2-fluoro-3-methyl-5- |
| | nitrobenzenesulfonamide |

Another aspect of the invention relates to the use of any of the intermediates described herein for preparing a compound of formula (I) as defined herein or an N-oxide, a salt, a hydrate, a solvate, a tautomer or a stereoisomer of said compound, or a salt of said N-oxide, tautomer or stereoisomer.

Preferred intermediates are the Intermediate Examples as disclosed below.

A further aspect of the invention are compounds of formula (I), (Ia) and (Ib) which are present as their salts.

It is to be understood that the present invention relates to any sub-combination within any embodiment or aspect of the present invention of compounds of general formula (I), (Ia) and (Ib) supra.

More particularly still, the present invention covers compounds of general formula (I), (Ia) and (Ib) which are disclosed in the Example section of this text, infra.

In accordance with another aspect, the present invention covers methods of preparing compounds of the present invention, said methods comprising the steps as described in the Experimental Section herein.

Another embodiment of the invention are compounds according to the claims as disclosed in the Claims section wherein the definitions are limited according to the preferred or more preferred definitions as disclosed below or specifically disclosed residues of the exemplified compounds and subcombinations thereof.

### Definitions

Constituents which are optionally substituted as stated herein, may be substituted, unless otherwise noted, one or more times, independently from one another at any possible position. When any variable occurs more than one time in any constituent, each definition is independent. For example, when R¹, R², R³, R⁴, R⁵, R⁶, R⁷, R⁸, R⁹, R¹⁰, R¹¹, R¹², X and/or Y occur more than one time in any compound of formula (I) each definition of R¹, R², R³, R⁴, R⁵, R⁶, R⁷, R⁸, R⁹, R¹⁰, R¹¹, R¹², X and Y is independent.

Should a constituent be composed of more than one part, e.g. C₁-C₄-alkoxy-C₁-C₄-alkyl-, the position of a possible substituent can be at any of these parts at any suitable position. A hyphen at the beginning of the constituent marks the point of attachment to the rest of the molecule. Should a ring be substituted the substitutent could be at any suitable position of the ring, also on a ring nitrogen atom if suitable.

Furthermore, a constituent composed of more than one part and comprising several chemical residues, e.g. C₁-C₄-alkoxy-C₁-C₄-alkyl or phenyl-C₁-C₄-alkyl, should be read from left to right with the point of attachment to the rest of the molecule on the last part (in the example mentioned previously on the C₁-C₄-alkyl residue)

The term “comprising” when used in the specification includes “consisting of”.

If it is referred to “as mentioned above” or “mentioned above” within the description it is referred to any of the disclosures made within the specification in any of the preceding pages.

“suitable” within the sense of the invention means chemically possible to be made by methods within the knowledge of a skilled person.

The terms as mentioned in the present text have preferably the following meanings:

The term “halogen”, “halogen atom”, “halo-” or “Hal-” is to be understood as meaning a fluorine, chlorine, bromine or iodine atom, preferably a fluorine or chlorine atom.

The term “C₁-C₄-alkyl” is to be understood as preferably meaning a linear or branched, saturated, monovalent hydrocarbon group having 1, 2, 3 or 4 carbon atoms, e.g. a methyl, ethyl, propyl, butyl, iso-propyl, iso-butyl, sec-butyl, tert-butyl group, particularly 1, 2 or 3 carbon atoms (“C₁-C₃-alkyl”), e.g. a methyl, ethyl, n-propyl- or iso-propyl group.

The term “C₁-C₄-haloalkyl” is to be understood as preferably meaning a linear or branched, saturated, monovalent hydrocarbon group in which the term “C₁-C₄-alkyl” is defined supra, and in which one or more hydrogen atoms is replaced by a halogen atom, in identically or differently, i.e. one halogen atom being independent from another. Particularly, said halogen atom is F. Said C₁-C₄-haloalkyl group is, for example, —CF₃, —CHF₂, —CH₂F, —CF₂CF₃, or —CH₂CF₃.

The term “C₁-C₄-alkoxy” is to be understood as preferably meaning a linear or branched, saturated, monovalent, hydrocarbon group of formula —O-alkyl, in which the term “alkyl” is defined supra, e.g. a methoxy, ethoxy, n-propoxy, iso-propoxy, n-butoxy, iso-butoxy, tert-butoxy or sec-butoxy group, or an isomer thereof.

The term “C₁-C₄-haloalkoxy” is to be understood as preferably meaning a linear or branched, saturated, monovalent C₁-C₄-alkoxy group, as defined supra, in which one or more of the hydrogen atoms is replaced, in identically or differently, by a halogen atom. Particularly, said halogen atom is F. Said C₁-C₄-haloalkoxy group is, for example, —OCF₃, —OCHF₂, —OCH₂F, —OCF₂CF₃, or —OCH₂CF₃.

The term “C₁-C₄-hydroxyalkyl” is to be understood as meaning a linear or branched, saturated, monovalent hydrocarbon group in which the term “C₁-C₄-alkyl” is defined supra, and in which one or more hydrogen atoms is replaced by a hydroxy group, e.g. a hydroxymethyl, 1-hydroxyethyl, 2-hydroxyethyl, 1,2-dihydroxyethyl, 3-hydroxypropyl, 2-hydroxypropyl, 2,3-dihydroxypropyl, 1,3-dihydroxypropan-2-yl, 3-hydroxy-2-methyl-propyl, 2-hydroxy-2-methyl-propyl, 1-hydroxy-2-methyl-propyl group.

The term “C₁-C₄-alkoxy-C₁-C₄-alkyl” is to be understood as preferably meaning a linear or branched, saturated, monovalent alkyl group, as defined supra, in which one or more of the hydrogen atoms is replaced, in identically or differently, by a C₁-C₄-alkoxy group, as defined supra, e.g. methoxyalkyl, ethoxyalkyl, propyloxyalkyl, iso-propoxyalkyl, butoxyalkyl, iso-butoxyalkyl, tert-butoxyalkyl or sec-butoxyalkyl group, in which the term “C₁-C₄-alkyl” is defined supra, or an isomer thereof.

The term “C₃-C₆-cycloalkyl” is to be understood as meaning a saturated, monovalent, mono-, or bicyclic hydrocarbon ring which contains 3, 4, 5 or 6 carbon atoms (“C₃-C₆-cycloalkyl”). Said C₃-C₆-cycloalkyl group is for example, a monocyclic hydrocarbon ring, e.g. a cyclopropyl, cyclobutyl, cyclopentyl or cyclohexyl, or a bicyclic hydrocarbon ring.

The term “4- to 6-membered heterocycloalkyl” or “4- to 6-membered heterocyclic ring”, is to be understood as meaning a saturated, monovalent, mono- or bicyclic hydrocarbon ring which contains 3, 4 or 5 carbon atoms, and one or more heteroatom-containing groups selected from C(═O), O, S, S(═O), S(═O)₂, NH, NR^{a}, in which R^{a} represents a C₁-C₆-alkyl- or C₁-C₆-haloalkyl- group; it being possible for said heterocycloalkyl group to be attached to the rest of the molecule via any one of the carbon atoms or, if present, the nitrogen atom.

Particularly, said heterocycloalkyl can contain 4 or 5 carbon atoms, and one or more of the above-mentioned heteroatom-containing groups (a “5- to 6-membered heterocycloalkyl”).

Particularly, without being limited thereto, said heterocycloalkyl can be a 4-membered ring, such as an azetidinyl, oxetanyl, or a 5-membered ring, such as tetrahydrofuranyl, dioxolinyl, pyrrolidinyl, imidazolidinyl, pyrazolidinyl, pyrrolinyl, or a 6-membered ring, such as tetrahydropyranyl, piperidinyl, morpholinyl, dithianyl, thiomorpholinyl, piperazinyl, or trithianyl, for example. Optionally, said heterocycloalkyl can be benzo fused.

The term “heteroaryl” is understood as preferably meaning a monovalent, monocyclic, bicyclic or tricyclic aromatic ring system having 5, 6, 7, 8, 9, 10, 11, 12, 13 or 14 ring atoms (a “5- to 14-membered heteroaryl” group), particularly 5, 6, 9 or 10 ring atoms, and which contains at least one heteroatom which may be identical or different, said heteroatom being such as oxygen, nitrogen or sulfur. In addition said ring system can be benzocondensed. Particularly, heteroaryl is selected from thienyl, furanyl, pyrrolyl, oxazolyl, thiazolyl, imidazolyl, pyrazolyl, isoxazolyl, isothiazolyl, oxadiazolyl, triazolyl, thiadiazolyl, thia-4H-pyrazolyl, and benzo derivatives thereof, such as, for example, benzofuranyl, benzothienyl, benzoxazolyl, benzisoxazolyl, benzimidazolyl, benzotriazolyl, indazolyl, indolyl, isoindolyl; or pyridinyl, pyridazinyl, pyrimidinyl, pyrazinyl, triazinyl, and benzo derivatives thereof, such as, for example, quinolinyl, quinazolinyl, isoquinolinyl; or azocinyl, indolizinyl, purinyl, and benzo derivatives thereof; or cinnolinyl, phthalazinyl, quinazolinyl, quinoxalinyl, naphthpyridinyl, pteridinyl, carbazolyl, acridinyl, phenazinyl, phenothiazinyl, phenoxazinyl, xanthenyl or oxepinyl.

In general, and unless otherwise mentioned, the heteroarylic radical include all the possible isomeric forms thereof, e.g. the positional isomers thereof. Thus, for some illustrative non-restricting example, the term pyridyl includes pyridin-2-yl, pyridin-3-yl and pyridin-4-yl; or the term thienyl includes thien-2-yl and thien-3-yl. Preferably, the heteroaryl group is a pyridyl group.

As mentioned supra, said nitrogen atom-containing ring can be partially unsaturated, i.e. it can contain one or more double bonds, such as, without being limited thereto, a 2,5-dihydro-1H-pyrrolyl, 4H-[1,3,4]thiadiazinyl, 4,5-dihydrooxazolyl, or 4H-[1,4]thiazinyl ring, for example, or, it may be benzo-fused, such as, without being limited thereto, a dihydroisoquinolinyl ring, for example.

The term “C₁-C₄”, as used throughout this text, e.g. in the context of the definition of “C₁-C₄-alkyl”, “C₁-C₄-haloalkyl”, “C₁-C₄-alkoxy”, or “C₁-C₄-haloalkoxy” is to be understood as meaning an alkyl group having a finite number of carbon atoms of 1 to 4, i.e. 1, 2, 3 or 4 carbon atoms. It is to be understood further that said term “C₁-C₄” is to be interpreted as any sub-range comprised therein, e.g. C₁-C₄, C₂-C₄, C₃-C₄, C₁-C₂, C₁-C₃, particularly C₁-C₂, C₁-C₃, C₁-C₄, in the case of “C₁-C₆-haloalkyl” or “C₁-C₄-haloalkoxy” even more particularly C₁-C₂.

Further, as used herein, the term “C₃-C₆”, as used throughout this text, e.g. in the context of the definition of “C₃-C₆-cycloalkyl”, is to be understood as meaning a cycloalkyl group having a finite number of carbon atoms of 3 to 6, i.e. 3, 4, 5 or 6 carbon atoms. It is to be understood further that said term “C₃-C₆” is to be interpreted as any sub-range comprised therein, e.g. C₃-C₆, C₄-C₅, C₃-C₅, C₃-C₄, C₄-C₆, C₅-C₆; particularly C₃-C₆.

The R⁹R¹⁰N—C(O)— group include, for example, —C(O)NH₂, —C(O)N(H)CH₃, —C(O)N(CH₃)₂, —C(O)N(H)CH₂CH₃, —C(O)N(CH₃)CH₂CH₃ or —C(O)N(CH₂CH₃)₂.

The R⁹R¹⁰N— group includes, for example, —NH₂, —N(H)CH₃, —N(CH₃)₂, —N(H)CH₂CH₃ and —N(CH₃)CH₂CH₃. In the case of R^{9a}R^{10a}N—, when R^{9a} and R^{10a} together with the nitrogen atom to which they are attached form a 4- to 6-membered nitrogen containing heterocyclic ring, said ring optionally containing one additional heteroatom selected from O, NH, NR^{a} in which R^{a} represents a C₁-C₆-alkyl- or C₁-C₆-haloalkyl- group, particularly a CH₃, or S and being optionally substituted, one to three times, independently from each other, with halogen or C₁-C₄-alkyl, particularly a CH₃.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

The term “optionally substituted” means optional substitution with the specified groups, radicals or moieties.

Ring system substituent means a substituent attached to an aromatic or nonaromatic ring system which, for example, replaces an available hydrogen on the ring system.

As used herein, the term “one or more”, e.g. in the definition of the substituents of the compounds of the general formulae of the present invention, is understood as meaning “one, two, three, four or five, particularly one, two, three or four, more particularly one, two or three, even more particularly one or two”.

The invention also includes all suitable isotopic variations of a compound of the invention. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁵I, ¹²⁹I and ¹³¹I, respectively. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

Where the plural form of the word compounds, salts, polymorphs, hydrates, solvates and the like, is used herein, this is taken to mean also a single compound, salt, polymorph, isomer, hydrate, solvate or the like.

By “stable compound’ or “stable structure” is meant a compound that is sufficiently robust to survive isolation to a useful degree of purity from a reaction mixture, and formulation into an efficacious therapeutic agent.

The compounds of this invention may contain one or more asymmetric centre, depending upon the location and nature of the various substituents desired. Asymmetric carbon atoms may be present in the (R) or (S) configuration, resulting in racemic mixtures in the case of a single asymmetric centre, and diastereomeric mixtures in the case of multiple asymmetric centres. In certain instances, asymmetry may also be present due to restricted rotation about a given bond, for example, the central bond adjoining two substituted aromatic rings of the specified compounds.

Substituents on a ring may also be present in either cis or trans form. It is intended that all such configurations (including enantiomers and diastereomers), are included within the scope of the present invention.

Preferred compounds are those which produce the more desirable biological activity. Separated, pure or partially purified isomers and stereoisomers or racemic or diastereomeric mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. R- or S-isomers, or E- or Z-isomers, in any ratio. Isolation of a single stereoisomer, e.g. a single enantiomer or a single diastereomer, of a compound of the present invention may be achieved by any suitable state of the art method, such as chromatography, especially chiral chromatography, for example.

Further, the compounds of the present invention may exist as tautomers. For example, any compound of the present invention which contains a pyrazole moiety as a heteroaryl group for example can exist as a 1H tautomer, or a 2H tautomer, or even a mixture in any amount of the two tautomers, or a triazole moiety for example can exist as a 1H tautomer, a 2H tautomer, or a 4H tautomer, or even a mixture in any amount of said 1H, 2H and 4H tautomers, namely:

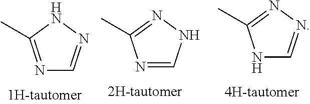

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also relates to useful forms of the compounds as disclosed herein, such as metabolites, hydrates, solvates, prodrugs, salts, in particular pharmaceutically acceptable salts, and co-precipitates.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the compounds of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol. Additionally, basic nitrogen containing groups may be quaternised with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and iodides, aralkyl halides like benzyl and phenethyl bromides and others.

Those skilled in the art will further recognise that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae such as “hydrochloride”, “trifluoroacetate”, “sodium salt”, or “x HCl”, “x CF3COOH”, “x Na+”, for example, are to be understood as not a stoichiometric specification, but solely as a salt form.

This applies analogously to cases in which synthesis intermediates or example compounds or salts thereof have been obtained, by the preparation and/or purification processes described, as solvates, such as hydrates with (if defined) unknown stoichiometric composition.

The salts include water-insoluble and, particularly, water-soluble salts.

Furthermore, derivatives of the compounds of formula (I) and the salts thereof which are converted into a compound of formula (I) or a salt thereof in a biological system (bioprecursors or pro-drugs) are covered by the invention. Said biological system is e.g. a mammalian organism, particularly a human subject. The bioprecursor is, for example, converted into the compound of formula (I) or a salt thereof by metabolic processes.

Furthermore, the present invention includes all possible crystalline forms, or polymorphs, of the compounds of the present invention, either as single polymorphs, or as a mixture of more than one polymorphs, in any ratio.

In the context of the properties of the compounds of the present invention the term “pharmacokinetic profile” means one single parameter or a combination thereof including permeability, bioavailability, exposure, and pharmacodynamic parameters such as duration, or magnitude of pharmacological effect, as measured in a suitable experiment. Compounds with improved pharmacokinetic profiles can, for example, be used in lower doses to achieve the same effect, may achieve a longer duration of action, or a may achieve a combination of both effects.

The term “combination” in the present invention is used as known to persons skilled in the art and may be present as a fixed combination, a non-fixed combination or kit-of-parts.

A “fixed combination” in the present invention is used as known to persons skilled in the art and is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A non-fixed combination or “kit-of-parts” in the present invention is used as known to persons skilled in the art and is defined as a combination wherein the said first active ingredient and the said second active ingredient are present in more than one unit. One example of a non-fixed combination or kit-of-parts is a combination wherein the said first active ingredient and the said second active ingredient are present separately. The components of the non-fixed combination or kit-of-parts may be administered separately, sequentially, simultaneously, concurrently or chronologically staggered. Any such combination of a compound of formula (I) of the present invention with an anti-cancer agent as defined below is an embodiment of the invention.

The term “(chemotherapeutic) anti-cancer agents”, includes but is not limited to 131I-chTNT, abarelix, abiraterone, aclarubicin, aldesleukin, alemtuzumab, alitretinoin, altretamine, aminoglutethimide, amrubicin, amsacrine, anastrozole, arglabin, arsenic trioxide, asparaginase, azacitidine, basiliximab, belotecan, bendamustine, bevacizumab, bexarotene, bicalutamide, bisantrene, bleomycin, bortezomib, buserelin, busulfan, cabazitaxel, calcium folinate, calcium levofolinate, capecitabine, carboplatin, carmofur, carmustine, catumaxomab, celecoxib, celmoleukin, cetuximab, chlorambucil, chlormadinone, chlormethine, cisplatin, cladribine, clodronic acid, clofarabine, copanlisib, crisantaspase, cyclophosphamide, cyproterone, cytarabine, dacarbazine, dactinomycin, darbepoetin alfa, dasatinib, daunorubicin, decitabine, degarelix, denileukin diftitox, denosumab, deslorelin, dibrospidium chloride, docetaxel, doxifluridine, doxorubicin, doxorubicin+estrone, eculizumab, edrecolomab, elliptinium acetate, eltrombopag, endostatin, enocitabine, epirubicin, epitiostanol, epoetin alfa, epoetin beta, eptaplatin, eribulin, erlotinib, estradiol, estramustine, etoposide, everolimus, exemestane, fadrozole, filgrastim, fludarabine, fluorouracil, flutamide, formestane, fotemustine, fulvestrant, gallium nitrate, ganirelix, gefitinib, gemcitabine, gemtuzumab, glutoxim, goserelin, histamine dihydrochloride, histrelin, hydroxycarbamide, I-125 seeds, ibandronic acid, ibritumomab tiuxetan, idarubicin, ifosfamide, imatinib, imiquimod, improsulfan, interferon alfa, interferon beta, interferon gamma, ipilimumab, irinotecan, ixabepilone, lanreotide, lapatinib, lenalidomide, lenograstim, lentinan, letrozole, leuprorelin, levamisole, lisuride, lobaplatin, lomustine, lonidamine, masoprocol, medroxyprogesterone, megestrol, melphalan, mepitiostane, mercaptopurine, methotrexate, methoxsalen, Methyl aminolevulinate, methyltestosterone, mifamurtide, miltefosine, miriplatin, mitobronitol, mitoguazone, mitolactol, mitomycin, mitotane, mitoxantrone, nedaplatin, nelarabine, nilotinib, nilutamide, nimotuzumab, nimustine, nitracrine, ofatumumab, omeprazole, oprelvekin, oxaliplatin, p53 gene therapy, paclitaxel, palifermin, palladium-103 seed, pamidronic acid, panitumumab, pazopanib, pegaspargase, PEG-epoetin beta (methoxy PEG-epoetin beta), pegfilgrastim, peginterferon alfa-2b, pemetrexed, pentazocine, pentostatin, peplomycin, perfosfamide, picibanil, pirarubicin, plerixafor, plicamycin, poliglusam, polyestradiol phosphate, polysaccharide-K, porfimer sodium, pralatrexate, prednimustine, procarbazine, quinagolide, radium-223 chloride, raloxifene, raltitrexed, ranimustine, razoxane, refametinib, regorafenib, risedronic acid, rituximab, romidepsin, romiplostim, roniciclib, sargramostim, sipuleucel-T, sizofiran, sobuzoxane, sodium glycididazole, sorafenib, streptozocin, sunitinib, talaporfin, tamibarotene, tamoxifen, tasonermin, teceleukin, tegafur, tegafur+gimeracil+oteracil, temoporfin, temozolomide, temsirolimus, teniposide, testosterone, tetrofosmin, thalidomide, thiotepa, thymalfasin, tioguanine, tocilizumab, topotecan, toremifene, tositumomab, trabectedin, trastuzumab, treosulfan, tretinoin, trilostane, triptorelin, trofosfamide, tryptophan, ubenimex, valrubicin, vandetanib, vapreotide, vemurafenib, vinblastine, vincristine, vindesine, vinflunine, vinorelbine, vorinostat, vorozole, yttrium-90 glass microspheres, zinostatin, zinostatin stimalamer, zoledronic acid, zorubicin.

It has now been found, and this constitutes the basis of the present invention, that said compounds of the present invention have surprising and advantageous properties.

In particular, compounds according to the present invention have surprisingly been found to effectively be active as an antagonist or a negative allosteric modulator of P2X4.

An allosteric modulator is a substance which indirectly influences (modulates) the effects of an agonist or inverse agonist at a target protein, for example a receptor. Allosteric modulators bind to a site distinct from that of the orthosteric agonist binding site. Usually they induce a conformational change within the protein structure. A negative modulator (NAM) reduces the effects of the orthosteric ligand, but is inactive in the absence of the orthosteric ligand.

### Commercial Utility and Medical Indications

As mentioned supra, the compounds of the present invention have surprisingly been found to effectively be active as an antagonist or a negative allosteric modulator of P2X4.

A compound according to the invention is used for the manufacture of a medicament.

A further aspect of the invention is the use of the compounds according to formula (I), (Ia) or (Ib) for the treatment or prophylaxis of a disease comprising administering an effective amount of a compound of formula (I), (Ia) or (Ib).

In accordance with an aspect of the present invention therefore the invention relates to a compound of general formula (I) (Ia) or (Ib), or an N-oxide, a salt, a tautomer or a stereoisomer of said compound, or a salt of said N-oxide, tautomer or stereoisomer particularly a pharmaceutically acceptable salt thereof, or a mixture of same, as described and defined herein, for use in the treatment or prophylaxis of a disease, especially for use in the treatment of a disease.

Preferably, the use of the compounds according to the present invention is in the treatment or prophylaxis of pain syndromes, especially the treatment, wherein the pain syndromes is related to endometriosis as well as for the treatment of endometriosis as such.

Another aspect is the use of a compound of formula (I), (Ia) or (Ib) is for the treatment of genitourinary, gastrointestinal, proliferative or pain-related disease, condition or disorder; cancer; fibrotic diseases including lung fibrosis, heart fibrosis, kidney fibrosis and fibrosis of other organs; gynaecological diseases including dysmenorrhea, dyspareunia, endometriosis and adenomyosis; endometriosis-associated pain; endometriosis-associated symptoms, wherein said symptoms are in particular endometriosis-associated proliferation, dysmenorrhea, dyspareunia, dysuria, or dyschezia; endometriosis-associated proliferation; pelvic hypersensitivity; urethritis; prostatitis; prostatodynia; cystitis; idiopathic bladder hypersensitivity; gastrointestinal disorders including irritable bowel syndrome (IBS), inflammatory bowel disease (IBD), biliary colic and other biliary disorders, renal colic, diarrhea-dominant IBS, gastroesophageal reflux, gastrointestinal distension, Crohn's disease and the like; atherosclerosis; lipid disorders; and pain-associated diseases selected from the group consisting of hyperalgesia, allodynia, functional bowel disorders (such as irritable bowel syndrome), arthritis (such as osteoarthritis and rheumatoid arthritis), burns, migraine or cluster headaches, nerve injury, neuritis, neuralgias, poisoning, ischemic injury, interstitial cystitis, cancer, traumatic nerve-injury, post-traumatic injuries (including fractures and sport injuries), trigeminal neuralgia, small fiber neuropathy, diabetic neuropathy, chronic arthritis and related neuralgias, HIV and HIV treatment-induced neuropathy, pruritus; impaired wound healing and disease of the skeleton like degeneration of the joints, ankylosing spondylitis (Burnstock et al., 2012 Pharmacol Rev. 64:834-868).

According to a particular aspect of the invention as reported above a compound of formula (I), (Ia) or (Ib) is for the treatment of pain syndromes (Trang and Salter, 2012, Purinergic Signalling 8:621-628; Burnstock, 2013 Eur J Pharmacol 716:24-40) including acute, chronic, inflammatory and neuropathic pain, preferably inflammatory pain, surgical pain, visceral pain, dental pain, premenstrual pain, endometriosis-associated pain, pain associated with fibrotic diseases, central pain, pain due to burning mouth syndrome, pain due to burns, pain due to migraine, cluster headaches, pain due to nerve injury, pain due to neuritis, neuralgias, pain due to poisoning, pain due to ischemic injury, pain due to interstitial cystitis, cancer pain, pain due to viral, parasitic or bacterial infections, pain due to traumatic nerve-injury, pain due to post-traumatic injuries (including fractures and sport injuries), pain due to trigeminal neuralgia, pain associated with small fiber neuropathy, pain associated with diabetic neuropathy, chronic lower back pain, phantom limb pain, pelvic pain syndrome, chronic pelvic pain, neuroma pain, complex regional pain syndrome, pain associated with gastrointestinal distension, chronic arthritic pain and related neuralgias, and pain associated with cancer, pain associated with chemotherapy, HIV and HIV treatment-induced neuropathy; and pain associated with diseases or disorders selected from the group consisting of hyperalgesia, allodynia, functional bowel disorders (such as irritable bowel syndrome) and arthritis (such as osteoarthritis and rheumatoid arthritis).

According to a further aspect of the invention as reported above a compound of formula (I), (Ia) or (Ib) is for the treatment of amyotrophic lateral sclerosis

Furthermore, a compound of formula (I), (Ia) or (Ib) according to the present invention is for use in the treatment of a gynecological disease, preferably dysmenorrhea, dyspareunia or endometriosis, adenomyosis, endometriosis-associated pain, or other endometriosis-associated symptoms, wherein said symptoms are in particular endometriosis-associated proliferation, dysmenorrhea, dyspareunia, dysuria, or dyschezia.

### Pharmaceutical Compositions of the Compounds of the Invention

This invention also relates to pharmaceutical compositions containing one or more compounds of the present invention. These compositions can be utilised to achieve the desired pharmacological effect by administration to a patient in need thereof. A patient, for the purpose of this invention, is a mammal, including a human, in need of treatment for the particular condition or disease.

Therefore, the present invention includes pharmaceutical compositions that are comprised of a pharmaceutically acceptable carrier or auxiliary and a pharmaceutically effective amount of a compound, or salt thereof, of the present invention.

Another aspect of the invention is a pharmaceutical composition comprising a pharmaceutically effective amount of a compound of formula (I) and a pharmaceutically acceptable auxiliary for the treatment of a disease mentioned supra, especially for the treatment of haematological tumours, solid tumours and/or metastases thereof.

A pharmaceutically acceptable carrier or auxiliary is preferably a carrier that is non-toxic and innocuous to a patient at concentrations consistent with effective activity of the active ingredient so that any side effects ascribable to the carrier do not vitiate the beneficial effects of the active ingredient. Carriers and auxiliaries are all kinds of additives assisting to the composition to be suitable for administration.

A pharmaceutically effective amount of compound is preferably that amount which produces a result or exerts the intended influence on the particular condition being treated.

The compounds of the present invention can be administered with pharmaceutically-acceptable carriers or auxiliaries well known in the art using any effective conventional dosage unit forms, including immediate, slow and timed release preparations, orally, parenterally, topically, nasally, ophthalmically, optically, sublingually, rectally, vaginally, and the like.

For oral administration, the compounds can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule that can be of the ordinary hard- or soft-shelled gelatine type containing auxiliaries, for example, surfactants, lubricants, and inert fillers such as lactose, sucrose, calcium phosphate, and corn starch.

In another embodiment, the compounds of this invention may be tableted with conventional tablet bases such as lactose, sucrose and cornstarch in combination with binders such as acacia, corn starch or gelatine, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, colouring agents, and flavouring agents such as peppermint, oil of wintergreen, or cherry flavouring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavouring and colouring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavouring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more colouring agents; one or more flavouring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavouring and colouring agents.

The compounds of this invention may also be administered parenterally, that is, subcutaneously, intravenously, intraocularly, intrasynovially, intramuscularly, or interperitoneally, as injectable dosages of the compound in preferably a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimise or eliminate irritation at the site of injection, such compositions may contain a non-ionic surfactant having a hydrophile-lipophile balance (HLB) preferably of from about 12 to about 17. The quantity of surfactant in such formulation preferably ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Controlled release formulations for parenteral administration include liposomal, polymeric microsphere and polymeric gel formulations that are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for administration, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized.

Such ingredients and procedures include those described in the following references, each of which is incorporated herein by reference: Powell, M. F. et al., “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)—Part-1” PDA Journal of Pharmaceutical Science & Technology 1999, 53(6), 324-349; and Nema, S. et al., “Excipients and Their Use in Injectable Products” PDA Journal of Pharmaceutical Science & Technology 1997, 51(4), 166-171.

Commonly used pharmaceutical ingredients that can be used as appropriate to formulate the composition for its intended route of administration include:

acidifying agents (examples include but are not limited to acetic acid, citric acid, fumaric acid, hydrochloric acid, nitric acid);
alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);
adsorbents (examples include but are not limited to powdered cellulose and activated charcoa)l;
aerosol propellants (examples include but are not limited to carbon dioxide, CCl₂F₂, F₂ClC—CClF₂ and CClF₃)
air displacement agents—examples include but are not limited to nitrogen and argon;
antifungal preservatives (examples include but are not limited to benzoic acid, butylparaben, ethylparaben, methylparaben, propylparaben, sodium benzoate);
antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);
antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);
binding materials (examples include but are not limited to block polymers, natural and synthetic rubber, polyacrylates, polyurethanes, silicones, polysiloxanes and styrene-butadiene copolymers);
buffering agents (examples include but are not limited to potassium metaphosphate, dipotassium phosphate, sodium acetate, sodium citrate anhydrous and sodium citrate dihydrate);
carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection);
chelating agents (examples include but are not limited to edetate disodium and edetic acid);
colourants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);
clarifying agents (examples include but are not limited to bentonite);
emulsifying agents (examples include but are not limited to acacia, cetomacrogol, cetyl alcohol, glyceryl monostearate, lecithin, sorbitan monooleate, polyoxyethylene 50 monostearate);
encapsulating agents (examples include but are not limited to gelatin and cellulose acetate phthalate),
flavourants (examples include but are not limited to anise oil, cinnamon oil, cocoa, menthol, orange oil, peppermint oil and vanillin);
humectants (examples include but are not limited to glycerol, propylene glycol and sorbitol);
levigating agents (examples include but are not limited to mineral oil and glycerin);
oils (examples include but are not limited to arachis oil, mineral oil, olive oil, peanut oil, sesame oil and vegetable oil);
ointment bases (examples include but are not limited to lanolin, hydrophilic ointment, polyethylene glycol ointment, petrolatum, hydrophilic petrolatum, white ointment, yellow ointment, and rose water ointment);
penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas),
plasticizers (examples include but are not limited to diethyl phthalate and glycerol);
solvents (examples include but are not limited to ethanol, corn oil, cottonseed oil, glycerol, isopropanol, mineral oil, oleic acid, peanut oil, purified water, water for injection, sterile water for injection and sterile water for irrigation);
stiffening agents (examples include but are not limited to cetyl alcohol, cetyl esters wax, microcrystalline wax, paraffin, stearyl alcohol, white wax and yellow wax);
suppository bases (examples include but are not limited to cocoa butter and polyethylene glycols (mixtures));
surfactants (examples include but are not limited to benzalkonium chloride, nonoxynol 10, octoxynol 9, polysorbate 80, sodium lauryl sulfate and sorbitan mono-palmitate);
suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);
sweetening agents (examples include but are not limited to aspartame, dextrose, glycerol, mannitol, propylene glycol, saccharin sodium, sorbitol and sucrose);
tablet anti-adherents (examples include but are not limited to magnesium stearate and talc);
tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);
tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);
tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);
tablet direct compression excipients (examples include but are not limited to dibasic calcium phosphate);
tablet disintegrant (examples include but are not limited to alginic acid, carboxymethylcellulose calcium, microcrystalline cellulose, polacrilin potassium, crosslinked polyvinylpyrrolidone, sodium alginate, sodium starch glycollate and starch);
tablet glidants (examples include but are not limited to colloidal silica, corn starch and talc);
tablet lubricants (examples include but are not limited to calcium stearate, magnesium stearate, mineral oil, stearic acid and zinc stearate);
tablet/capsule opaguants (examples include but are not limited to titanium dioxide);
tablet polishing agents (examples include but are not limited to carnuba wax and white wax);
thickening agents (examples include but are not limited to beeswax, cetyl alcohol and paraffin);
tonicity agents (examples include but are not limited to dextrose and sodium chloride);
viscosity increasing agents (examples include but are not limited to alginic acid, bentonite, carbomers, carboxymethylcellulose sodium, methylcellulose, polyvinyl pyrrolidone, sodium alginate and tragacanth); and
wetting agents (examples include but are not limited to heptadecaethylene oxycetanol, lecithins, sorbitol monooleate, polyoxyethylene sorbitol monooleate, and polyoxyethylene stearate).

Pharmaceutical compositions according to the present invention can be illustrated as follows:

Sterile i.v. solution: A 5 mg/ml solution of the desired compound of this invention can be made using sterile, injectable water, and the pH is adjusted if necessary. The solution is diluted for administration to 1-2 mg/ml with sterile 5% dextrose and is administered as an i.v. infusion over about 60 minutes.
Lyophilised powder for i.v. administration: A sterile preparation can be prepared with (i) 100-1000 mg of the desired compound of this invention as a lyophilised powder, (ii) 32-327 mg/ml sodium citrate, and (iii) 300-3000 mg Dextran 40. The formulation is reconstituted with sterile, injectable saline or dextrose 5% to a concentration of 10 to 20 mg/ml, which is further diluted with saline or dextrose 5% to 0.2-0.4 mg/ml, and is administered either IV bolus or by IV infusion over 15-60 minutes.
Intramuscular suspension: The following solution or suspension can be prepared, for intramuscular injection:
50 mg/ml of the desired, water-insoluble compound of this invention
5 mg/ml sodium carboxymethylcellulose
4 mg/ml TWEEN 80
9 mg/ml sodium chloride
9 mg/ml benzyl alcohol
Hard Shell Capsules: A large number of unit capsules are prepared by filling standard two-piece hard galantine capsules each with 100 mg of powdered active ingredient, 150 mg of lactose, 50 mg of cellulose and 6 mg of magnesium stearate.
Soft Gelatin Capsules: A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.
Tablets: A large number of tablets are prepared by conventional procedures so that the dosage unit is 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.
Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

### Dose and Administration

Based upon standard laboratory techniques known to evaluate compounds useful for the treatment of pain syndromes, and particularly in endometriosis, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### Combination Therapies

The term “combination” in the present invention is used as known to persons skilled in the art and may be present as a fixed combination, a non-fixed combination or kit-of-parts.

A “fixed combination” in the present invention is used as known to persons skilled in the art and is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A non-fixed combination or “kit-of-parts” in the present invention is used as known to persons skilled in the art and is defined as a combination wherein the said first active ingredient and the said second active ingredient are present in more than one unit. One example of a non-fixed combination or kit-of-parts is a combination wherein the said first active ingredient and the said second active ingredient are present separately. The components of the non-fixed combination or kit-of-parts may be administered separately, sequentially, simultaneously, concurrently or chronologically staggered.

The compounds of this invention can be administered as the sole pharmaceutical agent or in combination with one or more other pharmaceutical agents where the combination causes no unacceptable adverse effects. The present invention relates also to such combinations.

Those combined pharmaceutical agents can be other agents having antiproliferative, antinociceptive and/or antiinflammatory effects such as for example for the treatment of haematological tumours, solid tumours and/or metastases thereof and/or agents for the treatment of different pain syndromes and/or undesired side effects. The present invention relates also to such combinations.

Other anti-hyper-proliferative agents suitable for use with the composition of the invention include but are not limited to those compounds acknowledged to be used in the treatment of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, especially (chemotherapeutic) anti-cancer agents as defined supra.

Furthermore, the compounds of this invention can be combined with known hormonal therapeutical agents.

In particular, the compounds of the present invention can be administered in combination or as comedication with hormonal contraceptives. Hormonal contraceptives are for example Combined Oral Contraceptives (COCs) or Progestin-Only-Pills (POPs) or hormone-containing devices.

COCs include but are not limited to birth control pills or a birth control method that includes a combination of an estrogen (estradiol) and a progestogen (progestin). The estrogenic part is in most of the COCs ethinyl estradiol. Some COCs contain estradiol or estradiol valerate.

Said COCs contain the progestins norethynodrel, norethindrone, norethindrone acetate, ethynodiol acetate, norgestrel, levonorgestrel, norgestimate, desogestrel, gestodene, drospirenone, dienogest, or nomegestrol acetate.

Birth control pills include for example but are not limited to Yasmin, Yaz, both containing ethinyl estradiol and drospirenone; Microgynon or Miranova containing levonorgestrel and ethinyl estradiol; Marvelon containing ethinyl estradiol and desogestrel; Valette containing ethinyl estradiol and dienogest; Belara and Enriqa containing ethinyl estradiol and chlormadinonacetate; Qlaira containing estradiol valerate and dienogest as active ingredients; and Zoely containing estradiol and normegestrol.

POPs are contraceptive pills that contain only synthetic progestogens (progestins) and do not contain estrogen. They are colloquially known as mini pills.

POPs include but are not limited to Cerazette containing desogestrel; and Micronor containing norethindrone.

Other Progeston-Only forms are intrauterine devices (IUDs), for example Mirena containing levonorgestrel or injectables, for example Depo-Provera containing medroxyprogesterone acetate.

A preferred embodiment of the present invention is the administration of a compound of general formula (I) in combination with a COC or a POP or other Progestin-Only forms as mentioned above.

Another preferred embodiment of the present invention is the administration of a compound of general formula (Ia) in combination with a COC or a POP or other Progestin-Only forms as mentioned above.

Methods of testing for a particular pharmacological or pharmaceutical property are well known to persons skilled in the art.

The example testing experiments described herein serve to illustrate the present invention and the invention is not limited to the examples given.

As will be appreciated by persons skilled in the art, the invention is not limited to the particular embodiments described herein, but covers all modifications of said embodiments that are within the spirit and scope of the invention as defined by the appended claims.

The following examples illustrate the invention in greater detail, without restricting it. Further compounds according to the invention, of which the preparation is not explicitly described, can be prepared in an analogous way.

The compounds, which are mentioned in the examples and the salts thereof represent preferred embodiments of the invention as well as a claim covering all subcombinations of the residues of the compound of formula (I) as disclosed by the specific examples.

The term “according to” within the experimental section is used in the sense that the procedure referred to is to be used “analogously to”.

### Synthesis of Compounds

The following schemes and general procedures illustrate general synthetic routes to the compounds of general formula (I) of the invention and are not intended to be limiting. It is obvious to the person skilled in the art that the order of transformations as exemplified in schemes 1 to 2 can be modified in various ways. The order of transformations exemplified in schemes 1 to 2 is therefore not intended to be limiting. In addition, interconversion of substituents, for example of residues R¹, R², R³, R⁴, R⁵, R^{5a} and R^{5b} can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts, Protective Groups in Organic Synthesis, 3rd edition, Wiley 1999).

All reagents used for the preparation of the compounds of the invention are commercially available, known in the literature or can be prepared as described.

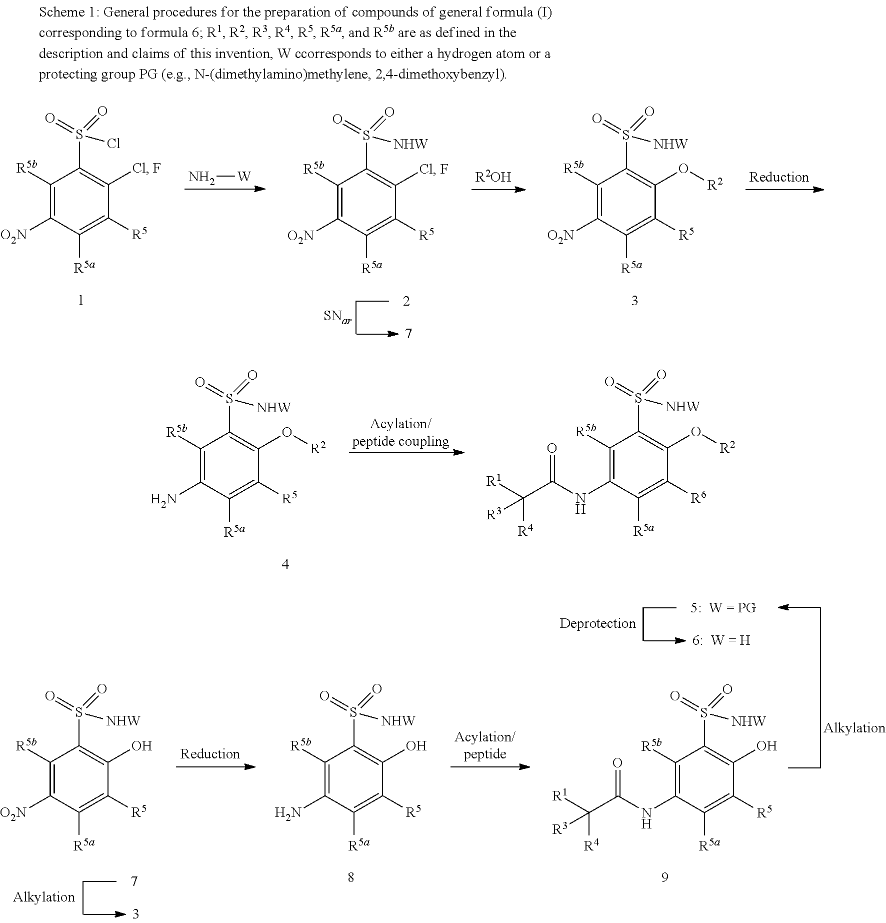

Compounds of general formula 6 can by synthesized as depicted in Scheme 1. Starting from the sulfonyl chloride the corresponding sulfonamides 2 can be obtained by reaction of ammonia or any amine in polar aprotic solvents such as dimethylformamide and acetonitrile. Subsequent nucleophilic aromatic substitution (SNₐᵣ) reaction with alcohols or phenols in the presence of a base, e.g. cesium carbonate or sodium hydride, in dimethylformamide or acetonitrile yield intermediates of general formula 3. Subsequent reduction under hydrogenation conditions, in polar solvents such as ethanol or tetrahydrofurane in the presence of for example Pd-, Pt- or Sn-based catalysts yield the aniline derivatives with general formula 4. Subsequent acylation to the corresponding amides for example by reaction with acyl chlorides or by standard peptide bond formation using all known procedures, such as reaction of the corresponding carboxylic acid in the presence of a coupling reagent e.g. HATU, and for W equals a protecting group subsequent deprotection with e.g. trifluoroacetic acid (TFA), results in compounds of general formula 6.

Alternatively starting from intermediate 7, which can be derived from intermediate 2 through reaction with hydroxide in various solvents such as DMF, alkylation with any alkylation reagent such as bromides in the presence of a base or reaction with the corresponding boronic acids in the presence of a suitable catalyst, e.g. copper(II)acetate (see for example Tetrahedron Letters, 1998, 39, 2937-2940.), leads to 3 and according to the procedures described above to final compounds with general formula 6. In addition intermediate 7 can be converted to the corresponding aniline derivatives 8 and by acylation and alkylations procedures, followed by deprotection (for W=PG) be converted to compounds 6.

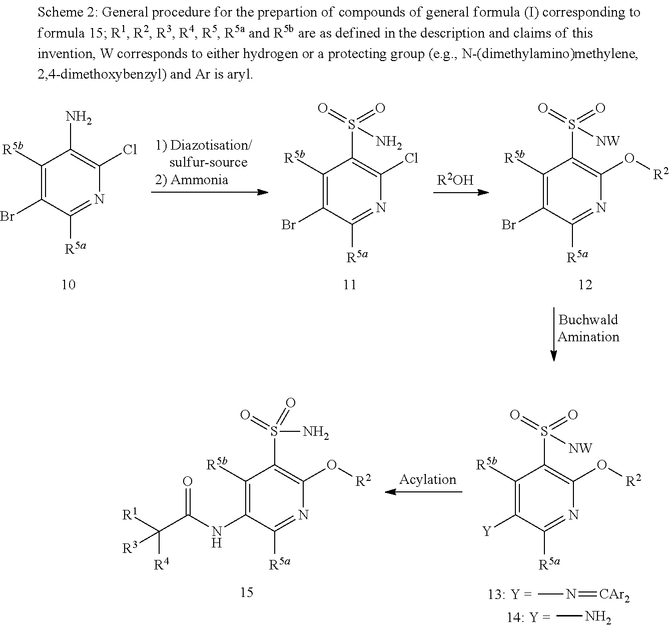

Diazotisation of 5-bromo-2-chloropyridin-3-amine using for example sodium nitrite in aqueous acid solution and subsequent transformation to the corresponding sulfonamide using a sulfonyl chloride source such as thionyl chloride in water followed by amination results in intermediate 11 (see for example J. Med. Chem., 2014, 57, 5, 2091-2106). Reaction with any nucleophiles undergoing aromatic nucleophilic substitution e.g. aromatic and aliphatic alcohols in the presence of base result in intermediate 12. Using protection and deprotection strategies, Buchwald amination in the presence of suitable catalysts (see for example WO2011120026A1) lead to intermediates 14 which can be converted to the final compounds of general formula 15 by acylation to the corresponding amides for example by reaction with acyl chlorides or by standard peptide bond formation using all known procedures, such as reaction of the corresponding carboxylic acid in the presence of a coupling reagent e.g. HATU.

The compounds according to the invention are isolated and purified in a manner known per se, e.g. by distilling off the solvent in vacuo and recrystallizing the residue obtained from a suitable solvent or subjecting it to one of the customary purification methods, such as chromatography on a suitable support material. Furthermore, reverse phase preparative HPLC of compounds of the present invention which possess a sufficiently basic or acidic functionality, may result in the formation of a salt, such as, in the case of a compound of the present invention which is sufficiently basic, a trifluoroacetate or formate salt for example, or, in the case of a compound of the present invention which is sufficiently acidic, an ammonium salt for example. Salts of this type can either be transformed into its free base or free acid form, respectively, by various methods known to the person skilled in the art, or be used as salts in subsequent biological assays. Additionally, the drying process during the isolation of compounds of the present invention may not fully remove traces of cosolvents, especially such as formic acid or trifluoroacetic acid, to give solvates or inclusion complexes. The person skilled in the art will recognise which solvates or inclusion complexes are acceptable to be used in subsequent biological assays. It is to be understood that the specific form (e.g. salt, free base, solvate, inclusion complex) of a compound of the present invention as isolated as described herein is not necessarily the only form in which said compound can be applied to a biological assay in order to quantify the specific biological activity.

Salts of the compounds of formula (I) according to the invention can be obtained by dissolving the free compound in a suitable solvent (for example a ketone such as acetone, methylethylketone or methylisobutylketone, an ether such as diethyl ether, tetrahydrofuran or dioxane, a chlorinated hydrocarbon such as methylene chloride or chloroform, or a low molecular weight aliphatic alcohol such as methanol, ethanol or isopropanol) which contains the desired acid or base, or to which the desired acid or base is then added. The acid or base can be employed in salt preparation, depending on whether a mono- or polybasic acid or base is concerned and depending on which salt is desired, in an equimolar quantitative ratio or one differing therefrom. The salts are obtained by filtering, reprecipitating, precipitating with a non-solvent for the salt or by evaporating the solvent.

Salts obtained can be converted into the free compounds which, in turn, can be converted into salts. In this manner, pharmaceutically unacceptable salts, which can be obtained, for example, as process products in the manufacturing on an industrial scale, can be converted into pharmaceutically acceptable salts by processes known to the person skilled in the art. Especially preferred are hydrochlorides and the process used in the example section.

Pure diastereomers and pure enantiomers of the compounds and salts according to the invention can be obtained e.g. by asymmetric synthesis, by using chiral starting compounds in synthesis and by splitting up enantiomeric and diasteriomeric mixtures obtained in synthesis.

Enantiomeric and diastereomeric mixtures can be split up into the pure enantiomers and pure diastereomers by methods known to a person skilled in the art. Preferably, diastereomeric mixtures are separated by crystallization, in particular fractional crystallization, or chromatography. Enantiomeric mixtures can be separated e.g. by forming diastereomers with a chiral auxiliary agent, resolving the diastereomers obtained and removing the chiral auxiliary agent. As chiral auxiliary agents, for example, chiral acids can be used to separate enantiomeric bases such as e.g. mandelic acid and chiral bases can be used to separate enantiomeric acids by formation of diastereomeric salts. Furthermore, diastereomeric derivatives such as diastereomeric esters can be formed from enantiomeric mixtures of alcohols or enantiomeric mixtures of acids, respectively, using chiral acids or chiral alcohols, respectively, as chiral auxiliary agents. Additionally, diastereomeric complexes or diastereomeric clathrates may be used for separating enantiomeric mixtures. Alternatively, enantiomeric mixtures can be split up using chiral separating columns in chromatography. Another suitable method for the isolation of enantiomers is the enzymatic separation.

One preferred aspect of the invention is the process for the preparation of the compounds of claims 1-6 according to the examples, as well as the intermediates used for their preparation.

Optionally, compounds of the formula (I) can be converted into their salts, or, optionally, salts of the compounds of the formula (I) can be converted into the free compounds.

Corresponding processes are customary for the skilled person.

### Experimental Part

### Abbreviations

The following table lists the abbreviations used in this paragraph and in the Intermediate Examples and Examples section as far as they are not explained within the text body.

| Abbreviation | Meaning |
|---|---|
| AcOH | acetic acid (ethanoic acid) |
| aq. | aqueous |
| avg | average |
| boc | t-butoxycarbonyl |
| br | broad |
| CI | chemical ionisation |
| d | doublet |
| DAD | diode array detector |
| DBU | 1,8-Diazabicyclo(5.4.0)undec-7-ene |
| DCM | dichloromethane |
| dd | double-doublet |
| DIPEA | diisopropylethylamine |
| DMF | N,N-dimethylformamide |
| DMSO | dimethyl sulfoxide |
| ELSD | Evaporative Light Scattering Detector |
| EtOAc | ethyl acetate |
| EtOH | ethanol |
| eq. | equivalent |
| ESI | electrospray (ES) ionisation |
| HATU | 1-[bis(dimethylamino)methylene]-1H-1,2,3-triazolo[4,5- |
| | b]pyridinium 3-oxid hexafluorophosphate |
| HPLC | high performance liquid chromatography |
| LC-MS | liquid chromatography mass spectrometry |
| m | multiplet |
| MeCN | acetonitrile |
| MeOH | methanol |
| MS | mass spectrometry |
| MTBE | methyl tert-butylether |
| NMR | nuclear magnetic resonance spectroscopy: chemical |
| | shifts (δ) are given in ppm. The chemical shifts were |
| | corrected by setting the DMSO signal to 2.50 ppm |
| | unless otherwise stated. |
| PDA | Photo Diode Array |
| PoraPak ™; | a HPLC column obtainable from Waters |
| q | quartet |
| quant. | quantitative |
| r.t. or rt | room temperature |
| Rt | retention time (as measured either with HPLC or UPLC) |
| | in minutes |
| s | singlet |
| SM | starting material |
| SQD | Single-Quadrupol-Detector |
| t | triplet |
| td | dublett of a triplet |
| | triplett of a dublet |
| TEA | triethylamine |
| THF | tetrahydrofuran |
| UPLC | ultra performance liquid chromatography |

Other abbreviations have their meanings customary per se to the skilled person.

The various aspects of the invention described in this application are illustrated by the following examples which are not meant to limit the invention in any way.

### Specific Experimental Descriptions

NMR peak forms in the following specific experimental descriptions are stated as they appear in the spectra, possible higher order effects have not been considered. Reactions employing microwave irradiation may be run with a Biotage Initator® microwave oven optionally equipped with a robotic unit. The reported reaction times employing microwave heating are intended to be understood as fixed reaction times after reaching the indicated reaction temperature. The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. from Separtis such as Isolute® Flash silica gel or Isolute® Flash NH₂ silica gel in combination with a Isolera® autopurifier (Biotage) and eluents such as gradients of e.g. hexane/ethyl acetate or DCM/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia. In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc) of a compound of the present invention as isolated as described herein is not necessarily the only form in which said compound can be applied to a biological assay in order to quantify the specific biological activity.

The percentage yields reported in the following examples are based on the starting component that was used in the lowest molar amount. Most reaction conditions were not optimized for yield. Air and moisture sensitive liquids and solutions were transferred via syringe or cannula, and introduced into reaction vessels through rubber septa.

Commercial grade reagents and solvents were used without further purification. The term “concentrated in vacuo” refers to use of a Buchi rotary evaporator at a minimum pressure of approximately 15 mm of Hg. All temperatures are reported uncorrected in degrees Celsius (° C.).

In order that this invention may be better understood, the following examples are set forth. These examples are for the purpose of illustration only, and are not to be construed as limiting the scope of the invention in any manner. All publications mentioned herein are incorporated by reference in their entirety.

### Analytical LC-MS and UPLC-MS Conditions

LC-MS and UPLC-MS data given in the subsequent specific experimental descriptions refer (unless otherwise noted) to the following conditions:

### Method A

instrument: Waters Acquity UPLC-MS SingleQuad; Column: Acquity UPLC BEH C18 1.7 μm, 50×2.1 mm; eluent A: water+0.1 vol % formic acid (99%), eluent B: acetonitrile; gradient: 0-1.6 min 1-99% B, 1.6-2.0 min 99% B; flow 0.8 ml/min; temperature: 60° C.; DAD scan: 210-400 nm.

### Method B

Instrument: Waters Acquity UPLC-MS SingleQuad; Column: Acquity UPLC BEH C18 1.7 μm, 50×2.1 mm; eluent A: water+0.2 vol % aqueous ammonia (32%), eluent B: acetonitrile; gradient: 0-1.6 min 1-99% B, 1.6-2.0 min 99% B; flow 0.8 ml/min; temperature: 60° C.; DAD scan: 210-400 nm.

### Method C

Instrument: Waters Acquity UPLC-MS SingleQuad; Column: Acquity UPLC BEH C18 1.7 μm, 50×2.1 mm; eluent A: water+0.1 vol % formic acid (99%), eluent B: acetonitrile; gradient: 0-1.7 min 1-45% B, 1.7-1.72 min 45-99% B, 1.72-2.0 min 99% B; flow 0.8 ml/min; temperature: 60° C.; DAD scan: 210-400 nm.

### Method D

Instrument: Waters Acquity UPLC-MS SingleQuad; Column: Acquity UPLC BEH C18 1.7 50×2.1 mm; eluent A: water+0.1 vol % formic acid (99%), eluent B: acetonitrile; gradient: 0-4.5 min 1-99% B, 4.5-5.0 min 99% B; flow 0.8 ml/min; temperature: 60° C.; DAD scan: 210-400 nm.

### Method E (Chiral HPLC)

Instrument: Agilent HPLC 1260; Säule: Chiralpak IA 3μ 100×4.6 mm; eluent A: hexan+0.1% vol. diethylamine (99%), eluent B: ethanol; isocratic: 60% A+40% B; flow 1.0 mL/min; temperature: 25° C.; injection: 5 μl; DAD @ 254 nm

### Method F

Instrument: Waters Acquity UPLC-MS SingleQuad; Column: Acquity UPLC BEH C18 1.7 μm, 50×2.1 mm; eluent A: water+0.1 vol % trifluoroacetic acid (99%), eluent B: acetonitrile; gradient: 0-1.6 min 1-99% B, 1.6-2.0 min 99% B; flow 0.8 ml/min; temperature: 60° C.; DAD scan: 210-400 nm.

### Method G

Instrument: Waters Acquity UPLC-MS SingleQuad; Column: Acquity UPLC BEH C18 1.7 μm, 50×2.1 mm; eluent A: water+0.2 vol % aqueous ammonia (32%), eluent B: acetonitrile; gradient: 0-4.5 min 5-95% B, 4.5-5.0 min 95% B; flow 0.8 ml/min; temperature: 50° C.; DAD scan: 210-400 nm.

### Method H

Instrument: Waters Acquity UPLCMS SingleQuad; Column: Acquity UPLC BEH C18 1.7 μm, 50×2.1 mm; eluent A: water+0.1 vol % formic acid (99%), eluent B: methanol; gradient: 0-1.6 min 1-99% B, 1.6-2.0 min 99% B; flow 0.8 ml/min; temperature: 60° C.; DAD scan: 210-400 nm.

### Method I

Instrument: Waters Acquity UPLCMS SingleQuad; Column: Acquity UPLC BEH C18 1.7 μm, 50×2.1 mm; eluent A: water+0.1 vol % trifluoroacetic acid, eluent B: acetonitrile; gradient: 0-4.5 min 5-95% B, 4.5-5.0 min 95% B; flow 0.8 ml/min; temperature: 50° C.; DAD scan: 210-400 nm.

### Method J

Instrument: Agilent 1290 UHPLCMS Tof; column: BEH C 18 (Waters) 1.7 μm, 50×2.1 mm; eluent A: water+0.05 Vol-% formic acid (99%), eluent B: acetonitrile+0.05% formic acid; gradient: 0-1.7 min 98-10% A, 1.7-2.0 min 10% A, 2.0-2.5 min 10-98% A, flow 1.2 ml/min; temperature: 60° C.; DAD scan: 210-400 nm.

### Flash Column Chromatography Conditions

“Purification by (flash) column chromatography” as stated in the subsequent specific experimental descriptions refers to the use of a Biotage Isolera purification system. For technical specifications see “Biotage product catalogue” on www.biotage.com.

### Determination of Optical Rotation Conditions

Optical rotations were measured using a JASCO P2000 Polarimeter at 589 nm wavelength, temperature 20° C., integration time 10 s and path length 100 mm. The solvent and concentration are specified in the examples.

### General Experimental Procedures

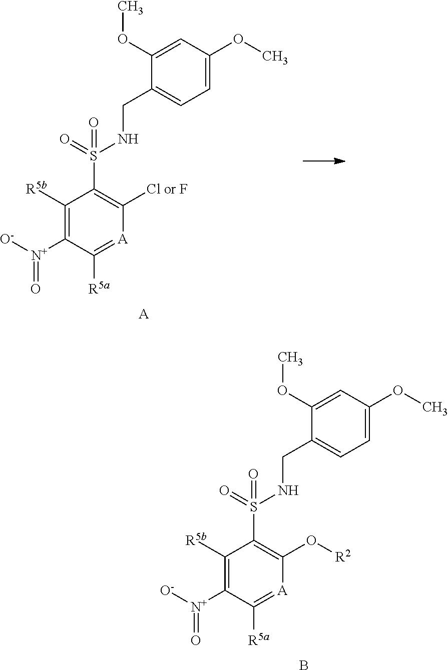

General Procedure GP1.1 (Nucleophilic Aromatic Substitution with Cesium Carbonate)

Sulfonamide A (1.29 mmol) was dissolved in acetonitrile (10 mL) and cesium carbonate (1.29 mmol, 1.0 eq) and the corresponding alcohol (1.29 mmol, 1.0 eq) were added. Stirring was continued at 85-100° C. until TLC showed consumption of starting material. The solvent was removed under reduced pressure, followed by addition of water and dichloromethane. Afterwards, the phases were separated, the organic phase was dried and it was concentrated in vacuo. The crude was either used without further purification or purified as indicated in the examples.

General Procedure GP1.2 (Nucleophilic Aromatic Substitution with Sodium Hydride)

Sulfonamide A (1.29 mmol) was dissolved in dimethylformamide (20 mL) and the corresponding alcohol (1.94 mmol, 1.5 eq) was added followed by the addition of sodium hydride (9.05 mmol, 1.5 eq). Stirring was continued for aliphatic alcohols at room temperature and for phenols at 110° C. until TLC showed consumption of starting material. The reaction mixture was cooled to 0° C. and water and ethyl acetate was carefully added. Afterwards, the phases were separated and the aqueous phase was extracted three times with ethyl acetate. The combined organic phases were dried and concentrated in vacuo. The crude was either used without further purification or purified as indicated in the examples.

General Procedure GP1.3 (Nucleophilic Aromatic Substitution with Potassium Carbonate)

Sulfonamide A (1.29 mmol) was dissolved in dimethylformamide (20 mL) and the corresponding alcohol (1.94 mmol, 1.5 eq) was added followed by the addition of potassium carbonate (9.05 mmol, 1.5 eq). Stirring was continued at 100° C. until TLC showed consumption of starting material. The reaction mixture was cooled to 0° C. and water and ethyl acetate was carefully added. Afterwards, the phases were separated and the aqueous phase was extracted three times with ethyl acetate. The combined organic phases were dried and concentrated in vacuo. The crude was either used without further purification or purified as indicated in the examples.

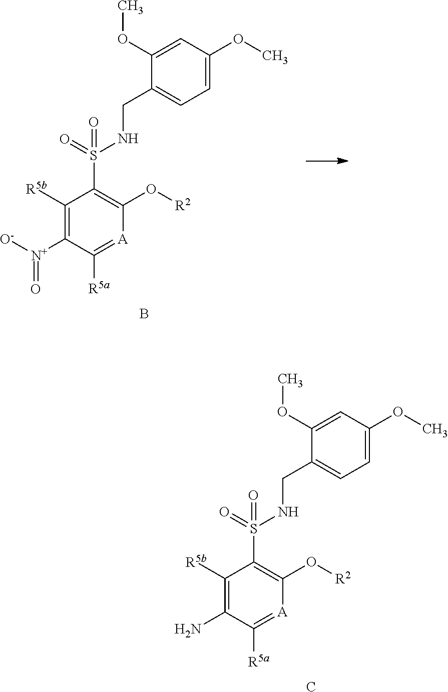

General Procedure GP2.1 (Reduction with Hydrogen on Pd/C)

Nitro compound B (0.85 mmol) was dissolved in tetrahydrofuran (25 mL) and Pd/C (0.09 mmol, 0.1 eq) was added. The flask was evacuated three times and flushed with hydrogen (1 bar) and stirring was continued at room temperature. After completion of the reaction, the mixture was filtered and concentrated in vacuo. The crude was used without further purification.

General Procedure GP2.2 (Reduction with Tin(II)Chloride Dehydrate)

Nitro compound B (1.29 mmol) was dissolved in dioxane (6 mL) and tin(II)chloride dihydrate (6.46 mmol, 5.0 eq) was added and the reaction mixture was stirred for 2 h at 70° C. After cooling to room temperature the reaction mixture was filtered and concentrated in vacuo. The filtrate was either used without further purification or purified as indicated in the examples.

General Procedure GP2.3 (Reduction with Iron)

Nitro compound B (2.6 mmol) was dissolved in tetrahydrofuran/methanol (40 mL 1/1 v/v) and added to a solution of ammonium chloride (13 mmol, 5.0 eq) and iron powder (13 mmol, 5.0 eq) in water (40 mL). The reaction mixture was heated for 2 h at 80-90° C. After cooling to room temperature the reaction mixture was filtered via Celite, washed with methanol and the filtrate was concentrated in vacuo. The crude was dissolved in ethyl acetate and the organic phase was washed with water. The aqueous phase was extracted three times with ethyl acetate, the combined organic phases were dried and concentrated in vacuo. The crude was used without further purification.

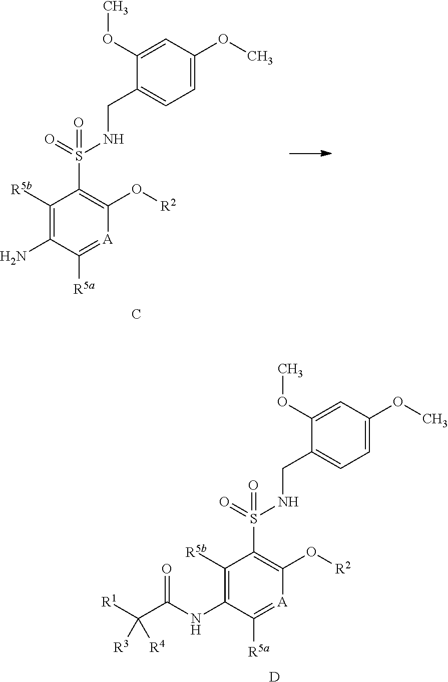

General Procedure GP3.1 (Acylation with HATU)

Amino compound C (0.17 mmol) was dissolved in dimethylformamide (5 mL) followed by the addition of the corresponding acid (0.2 mmol), N,N-diisopropylethylamine (0.15 mL, 0.8 mmol) and HATU (131 mg, 0.33 mmol). The reaction mixture was either stirred overnight at room temperature or heated at 50° C. until TLC showed consumption of starting material. After cooling to r.t. ethyl acetate and water were added to the reaction mixture and phases were separated. The aqueous phase was extracted three times with ethyl acetate and the combined organic phase was dried and the solvent was removed under reduced pressure. The crude was used without further purification.

General Procedure GP3.2 (Acylation with HATU)

Substituted aniline C (1.29 mmol) was dissolved in dimethylformamide (6 mL) followed by the addition of the corresponding acid (1.42 mmol, 1.1 eq), N,N-diisopropylethylamine (6.46 mmol, 5.0 eq) and HATU (2.07 mmol, 1.6 eq). The reaction mixture was either stirred overnight at room temperature or heated at 50° C. until TLC showed consumption of starting material. After cooling to room temperature the reaction mixture was concentrated in vacuo. Ethyl acetate and water were added, the organic phase was dried and concentrated in vacuo. The crude was used without further purification.

General Procedure GP3.3 (Acylation with HATU)

Substituted aniline C (0.25 mmol), the corresponding acid (0.50 mmol, 2.0 eq), HATU (0.50 mmol, 2.0 eq) and N-methylmorpholine (1.0 mmol, 2.0 eq) were dissolved in NMP (2.83 mL, containing 2.5% DMAP) and were stirred for 2 h at room temperature, followed by stirring overnight at 60° C. The reaction mixture was concentrated in vacuo and the crude was used without further purification.

General Procedure GP3.4 (Acylation with Acid Chlorides)

Amino compound C (0.17 mmol) was dissolved in dimethylformamide (5 mL) followed by the addition of the corresponding acid chloride (0.6 mmol), potassium carbonate (0.5 mmol). The reaction mixture was stirred at room at 100° C. until TLC showed consumption of starting material. After cooling to r.t. dichloromethane and water were added to the reaction mixture and phases were separated. The aqueous phase was extracted three times with ethyl acetate and the combined organic phase was dried and the solvent was removed under reduced pressure. The crude was used without further purification.

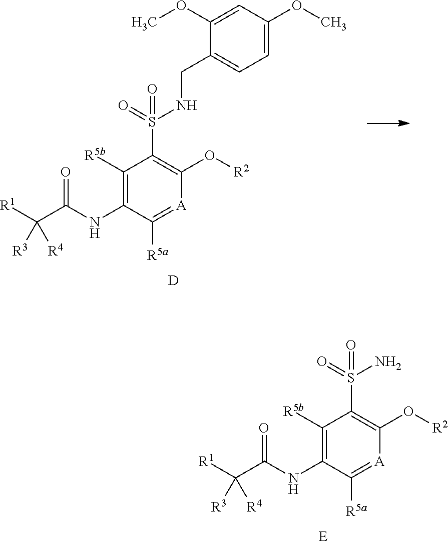

### General Procedure GP4 (Deprotection of 2,4-Dimethoxybenzyl Sulfonamides)

Crude amide D (1.29 mmol) was dissolved in dichloromethane (5-10 mL), trifluoroacetic acid (64.5 mmol, 50 eq) was added and the reaction mixture was stirred at room temperature until TLC showed consumption of starting material. The reaction mixture was concentrated in vacuo, ethyl acetate and water were added to the crude and the organic phase was dried and the solvent was removed under reduced pressure. The resulting residue was purified as indicated in the examples. Purification without aqueous extraction was also possible but made the HPLC purification more difficult.

### General Procedure GP5 (Alkylation of Hydroxyarylsulfonamides)

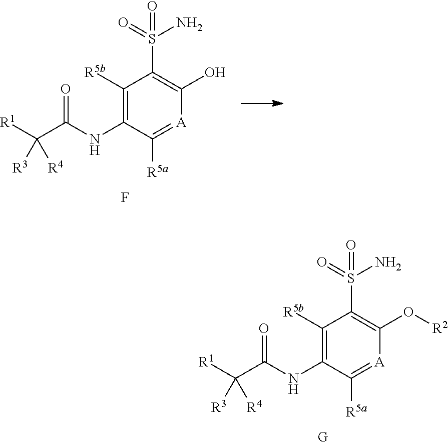

Substituted phenol F (0.20 mmol) was dissolved in dimethyl formamide (3-5 mL), cooled in an ice bath and treated with sodium hydride (55% purity, 0.24 mmol, 1.2 eq). After stirring for 20 min the corresponding alkyl or benzyl halide (0.30 mmol, 1.5 eq) was added and the reaction mixture was allowed to warm up and was stirred at room temperature (if not indicated otherwise) until TLC showed consumption of starting material. Water and ethyl acetate were added, the organic phase was washed twice with water, dried and concentrated in vacuo. The crude was purified as indicated in the examples to yield pure final compound.

### Synthesis of Intermediates

### Intermediate 001

### 2-Chloro-N-(2,4-dimethoxybenzyl)-5-nitrobenzenesulfonamide

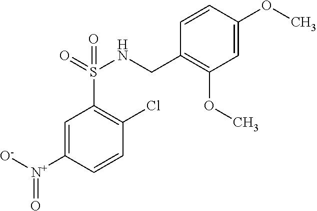 [SMILES: COc1ccc(CNS(=O)(=O)c2cc([N+](=O)[O-])ccc2Cl)c(OC)c1]

To a solution of 2-chloro-5-nitrobenzenesulfonamide (10.8 g, 42.2 mmol) in dichloromethane (108 mL) was added sodium bicarbonate (7.09 g, 84.4 mmol) and 1-(2,4-dimethoxyphenyl)methanamine (7.05 g, 42.2 mmol). The mixture was stirred overnight. The reaction mixture was concentrated in vacuo, followed by addition of water (75 mL) and ethyl acetate (75 mL). After stirring for 10 min the resulting precipitate was separated by filtration and it was dried at 40° C. overnight in vacuo to yield 2-chloro-N-(2,4-dimethoxybenzyl)-5-nitrobenzenesulfonamide (14.1 g, 36.5 mmol, 86% yield).

LC-MS (Method A): Rt=1.17 min

MS (ESIneg): m/z=385 (M−H)⁺

¹H-NMR (400 MHz, DMSO-d₆) δ [ppm]: 3.56 (s, 3H), 3.61 (s, 3H), 4.08 (s, 2H), 6.10 (d, 1H), 6.26 (dd, 1H), 7.04 (d, 1H), 7.79 (d, 1H), 8.19 (d, 1H), 8.28 (dd, 1H), 8.45 (s, 1H).

### Intermediate 002

### N-(2,4-Dimethoxybenzyl)-2-fluoro-5-nitrobenzenesulfonamide

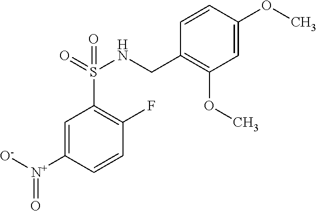 [SMILES: COc1ccc(CNS(=O)(=O)c2cc([N+](=O)[O-])ccc2F)c(OC)c1]

To a solution of 1-(2,4-dimethoxyphenyl)methanamine (0.669 g, 4.00 mmol) in dichloromethane (40 mL) was added under ice cooling N-ethyl-N-isopropylpropan-2-amine (1.29 g, 10.0 mmol). Over 25 min a solution of 2-fluoro-5-nitrobenzenesulfonyl chloride (0.958 g, 4.00 mmol) in dichloromethane (10 mL) was slowly added. Stirring was continued under ice cooling for 2 h, followed by stirring at room temperature overnight. It was washed with water, dried over sodium sulfate and concentrated in vacuo. Column chromatography on a Biotage Isolera system (silica gel, gradient n-hexane/ethyl acetate) gave N-(2,4-dimethoxybenzyl)-2-fluoro-5-nitrobenzenesulfonamide (400 mg, 1.08 mmol, 27% yield, purity 70%).

LC-MS (Method A): Rt=1.12 min

MS (ESIneg): m/z=369 (M−H)⁺

### Intermediate 003

### 2,4-Dichloro-N-(2,4-dimethoxybenzyl)-5-nitrobenzenesulfonamide

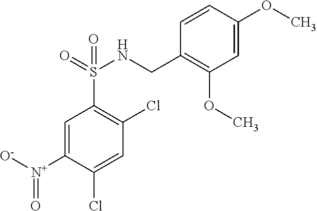 [SMILES: COc1ccc(CNS(=O)(=O)c2cc([N+](=O)[O-])c(Cl)cc2Cl)c(OC)c1]

To a suspension of 2,4-dichloro-5-nitrobenzenesulfonyl chloride (900 mg, 3.10 mmol) and sodium bicarbonate (521 mg, 6.20 mmol) in dichloromethane (10 mL) was added at 0° C. a solution of 1-(2,4-dimethoxyphenyl)methanamine (518 mg, 3.10 mmol) in dichloromethane (10 mL). The reaction was stirred overnight at room temperature, water was added and the organic phase was separated and dried over sodium sulfate. Concentration in vacuo gave crude 2,4-dichloro-N-(2,4-dimethoxybenzyl)-5-nitrobenzenesulfonamide (1.30 g, quant.) which was used without further purification in the next steps.

LC-MS (Method A): Rt=1.26 min

MS (neg): m/z=419 (M−H)⁺

### Intermediate 004

### N-(2,4-Dimethoxybenzyl)-2,3-difluoro-5-nitrobenzenesulfonamide

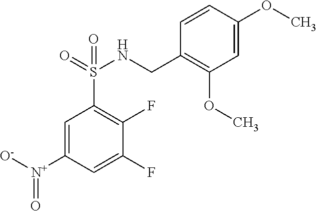 [SMILES: COc1ccc(CNS(=O)(=O)c2cc([N+](=O)[O-])cc(F)c2F)c(OC)c1]

To a solution of 2,3-difluoro-5-nitrobenzenesulfonyl chloride (5.0 g, 19.3 mmol) in dichloromethane (50 mL) was added sodium bicarbonate (3.25 g, 38.6 mmol) and 1-(2,4-dimethoxyphenyl)methanamine (3.23 g, 19.3 mmol). The mixture was washed with water and extracted with ethyl acetate, the organic phases were dried over sodium sulfate and concentrated in vacuo. Crystallization from n-hexane/ethyl acetate gave N-(2,4-dimethoxybenzyl)-2,3-difluoro-5-nitrobenzenesulfonamide (3.25 g, 8.37 mmol, 43% yield, 99% purity).

LC-MS (Method A): Rt=1.48 min

MS (neg): m/z=387 (M−H)⁺

### Intermediate 005

### 2-(3-Chlorophenoxy)-N-(2,4-dimethoxybenzyl)-3-fluoro-5-nitrobenzenesulfonamide

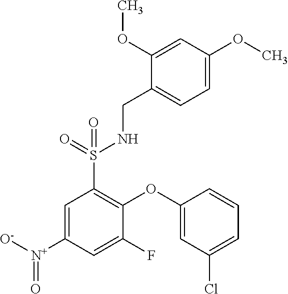 [SMILES: COc1ccc(CNS(=O)(=O)c2cc([N+](=O)[O-])cc(F)c2Oc2cccc(Cl)c2)c(OC)c1]

Cesium carbonate (2.73 g, 8.38 mmol) and 3-chlorophenol (1.08 g, 8.38 mmol) were added at 0° C. to a solution of N-(2,4-Dimethoxybenzyl)-2,3-difluoro-5-nitrobenzenesulfonamide (3.25 g, 8.38 mmol) in acetonitrile (50 mL), followed by stirring at room temperature until TLC showed consumption of starting material. The mixture was washed with water and extracted with ethyl acetate, the organic phases were dried over sodium sulfate and concentrated in vacuo to give crude 2-(3-chlorophenoxy)-N-(2,4-dimethoxybenzyl)-3-fluoro-5-nitrobenzenesulfonamide in quantitative yield (4.16 g, 8.38 mmol).

LC-MS (Method A): Rt=1.60 min

MS (neg): m/z=495 (M−H)⁺

### Intermediate 006

### 5-Amino-2-(3-chlorophenoxy)-3-fluorobenzenesulfonamide

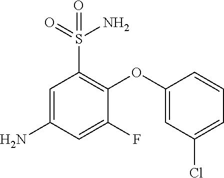 [SMILES: Nc1cc(F)c(Oc2cccc(Cl)c2)c(S(N)(=O)=O)c1]

To a solution of crude 2-(3-chlorophenoxy)-N-(2,4-dimethoxybenzyl)-3-fluoro-5-nitrobenzenesulfonamide (4.16 g, 8.38 mmol) in dioxane (150 mL) was slowly added tin(II)chloride dihydrate (10.2 g, 45.1 mmol). After stirring at room temperature overnight, the mixture was washed with water and extracted with ethyl acetate, the organic phases were dried over sodium sulfate and concentrated in vacuo. Chromatography on a Biotage Isolera system (silica gel, gradient n-hexane/ethyl acetate gradient) led to 5-amino-2-(3-chlorophenoxy)-3-fluorobenzenesulfonamide of around 70% purity which was used without further purification in the following acylation step.

LC-MS (Method A): Rt=1.12 min

MS (pos): m/z=317 (M+H)⁺

### Intermediate 007

### 5-Amino-2-hydroxybenzenesulfonamide

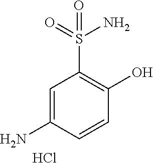 [SMILES: Nc1ccc(O)c(S(N)(=O)=O)c1 ; Cl]

To a solution of 2-hydroxy-5-nitrobenzenesulfonamide (10.9 g, 50.0 mmol) in methanol (250 mL) was added under argon aqueous 2M-HCl (25 mL, 50 mmol) and 10% Pd/C (1.5 g). After stirring under an atmosphere of hydrogen for 20 h, the catalyst was removed by filtration over a PTFE-membrane and the filtrate was concentrated in vacuo affording crude 5-amino-2-hydroxybenzenesulfonamide hydrochloride that was used in the next steps without further purification (11.2 g, 0.499 mmol, 99% yield, 95% purity).

LC-MS (Method C): Rt=0.20 min

MS (ESIneg): m/z=187 (M−H)⁺

¹H-NMR (400 MHz, deuterium oxide) δ [ppm]: 7.23 (d, 1H), 7.57 (dd, 1H), 7.83 (d, 1H).

### Intermediate 008

### 2-(2-Chlorophenyl)-N-(4-hydroxy-3-sulfamoylphenyl)acetamide

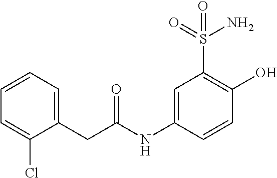 [SMILES: NS(=O)(=O)c1cc(NC(=O)Cc2ccccc2Cl)ccc1O]

To a suspension of 5-amino-2-hydroxybenzenesulfonamide hydrochloride (2.25 g, 10.0 mmol) in tetrahydrofuran (75 mL) was added (2-chlorophenyl)acetic acid (1.88 g, 11 mmol), N,N-diisopropylethylamine (6.46 g, 50 mmol) and HATU (4.18 g, 11 mmol). The reaction mixture was stirred overnight at room temperature. Then it was concentrated in vacuo, followed by extraction from ethyl acetate/water. The organic phase was washed with water, dried over sodium sulfate and concentrated in vacuo. Column chromatography on a Biotage Isolera system (silica gel, gradient dichloromethane to dichloromethane/methanol 80/20) led to 2-(2-chlorophenyl)-N-(4-hydroxy-3-sulfamoylphenyl)acetamide (1.60 g, 4.70 mmol, 47% yield, 90% purity).

LC-MS (Method A): Rt=0.85 min

MS (ESIpos): m/z=341 (M+H)⁺

¹H NMR (400 MHz, DMSO-d₆) δ [ppm]: 3.78 (s, 2H), 6.85-6.95 (m, 3H), 7.25-7.33 (m, 2H), 7.36-7.46 (m, 2H), 7.59 (dd, 1H), 7.93 (d, 1H), 10.17 (s, 1H), 10.39 (s, 1H).

### Intermediate 009

### 2-(2-Chloro-3-fluorophenyl)-N-(4-hydroxy-3-sulfamoylphenyl)acetamide

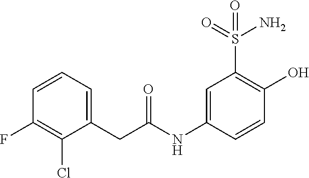 [SMILES: NS(=O)(=O)c1cc(NC(=O)Cc2cccc(F)c2Cl)ccc1O]

To a suspension of 5-amino-2-hydroxybenzenesulfonamide hydrochloride (0.450 g, 2.00 mmol) in tetrahydrofuran (20 mL) was added (2-chloro-3-fluorophenyl)acetic acid (0.415 g, 2.20 mmol), N,N-diisopropylethylamine (1.29 g, 10.0 mmol) and HATU (0.837 g, 2.20 mmol). The reaction mixture was stirred overnight at room temperature, followed by concentration in vacuo and extraction from ethyl acetate/water. The organic phase was washed with water, dried over sodium sulfate and concentrated in vacuo. As LC-MS showed mostly bisacylated product the residue was redissolved in tetrahydrofuran and treated for 24 h with aqueous 1M-NaOH (15 mL). After removing tetrahydrofuran in vacuo, it was neutralized with diluted hydrochloric acid, followed by extraction with ethyl acetate. The organic phase was washed with water, dried over sodium sulfate and concentrated in vacuo. Purification by preparative HPLC (Chromatorex C-18 10 μm, 125×30 mm, acetonitrile/water+0.1% formic acid) led to 2-(2-chloro-3-fluorophenyl)-N-(4-hydroxy-3-sulfamoylphenyl)acetamide (240 mg, 0.669 mmol, 33% yield, 99% purity).

LC-MS (Method A): Rt=0.86 min

MS (ESIpos): m/z=359 (M+H)⁺

¹H-NMR (400 MHz, DMSO-d₆) δ [ppm]: 3.86 (s, 2H), 6.90-7.00 (m, 3H), 7.24-7.41 (m, 3H), 7.60 (dd, 1H), 7.94 (d, 1H), 10.24 (s, 1H), 10.45 (s, 1H).

### Intermediate 010

### 2-(2-Chloro-6-fluorophenyl)-N-(4-hydroxy-3-sulfamoylphenyl)acetamide

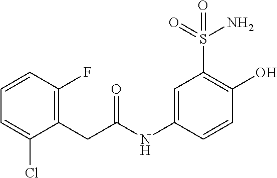 [SMILES: NS(=O)(=O)c1cc(NC(=O)Cc2c(F)cccc2Cl)ccc1O]

To a suspension of 5-amino-2-hydroxybenzenesulfonamide hydrochloride (1.24 g, 5.50 mmol) in tetrahydrofuran (55 mL) was added (2-chloro-6-fluorophenyl)acetic acid (1.14 g, 6.05 mmol), N,N-diisopropylethylamine (3.55 g, 27.5 mmol) and HATU (2.3 g, 6.05 mmol). The reaction mixture was stirred over a weekend at room temperature, followed by concentration in vacuo and extraction from ethyl acetate/water. The organic phase was washed with water, dried over sodium sulfate and concentrated in vacuo. As LC-MS showed mostly bisacylated product the residue was redissolved in tetrahydrofuran and treated for 24 h with aqueous 1M-NaOH (30 mL). After removing tetrahydrofuran in vacuo, it was neutralized with diluted hydrochloric acid, followed by extraction with ethyl acetate. The organic phase was washed with water, dried over sodium sulfate and concentrated in vacuo. Purification by preparative HPLC (Chromatorex C-18 10 μm, 125×30 mm, acetonitrile/water+0.1% formic acid) led to 2-(2-chloro-6-fluorophenyl)-N-(4-hydroxy-3-sulfamoylphenyl)acetamide (55 mg, 0.153 mmol, 3% yield, 99% purity).

LC-MS (Method A): Rt=0.85 min

MS (ESIpos): m/z=359 (M+H)⁺

¹H-NMR (400 MHz, DMSO-d₆) δ [ppm]: 3.85 (d, 2H), 6.80-7.05 (m, 3H), 7.21-7.27 (m, 1H), 7.32-7.42 (m, 2H), 7.58 (dd, 1H), 7.93 (d, 1H), 10.29 (s, 1H), 10.35-10.68 (m, 1H).

### Intermediate 011

### 5-Bromo-2-hydroxypyridine-3-sulfonamide

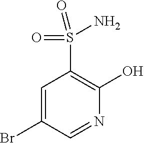 [SMILES: NS(=O)(=O)c1cc(Br)cnc1O]

Water (50 mL) was cooled to 0° C. and within 1 h thionyl chloride (13.6 g, 119 mmol) was carefully added. The reaction mixture was allowed to warm to room temperature and stirring was continued overnight. Copper(I)chloride was added and the reaction mixture was cooled to −3° C.

In a separate flask, concentrated hydrochloric acid (27.2 mL) was carefully added under ice cooling to 3-amino-5-bromopyridin-2-ol (3.50 g, 16.9 mmol) at a speed that the temperature stayed below 30° C. After stirring for 15 min at that temperature it was cooled to −5° C. and a solution of sodium nitrite (2.05 g, 29.7 mmol) in water (8 mL) was added over 45 min while the temperature was kept between −5 and 0° C. Stirring was continued for 10 min at −5° C., then this orange suspension was slowly added over 30 min at −5 to 0° C. to the “thionyl chloride solution” from the beginning. Stirring was continued at 0° C. for 75 min and the white precipitate was isolated by filtration, resulting in 3.5 g crude sulfonyl chloride.

This crude sulfonyl chloride was dissolved in methanol (300 mL) and ammonia in methanol (4.20 mL, 33%) was slowly added. Stirring was continued for 1 h before concentration in vacuo. The residue was stirred in n-hexane/ethyl acetate (1/1) and the precipitate was filtered off. 5-Bromo-2-hydroxypyridine-3-sulfonamide was obtained in sufficient purity by removing the solvent of the mother liquor under reduced pressure (953 mg, 5.51 mmol, 21% yield, 95% purity)

LC-MS (Method A): Rt=0.50 min

MS (ESIpos): m/z=274 (M+H)⁺

¹H-NMR (400 MHz, DMSO-d₆) δ [ppm]: 7.95 (s, 2H), 8.45 (d, 1H), 8.80 (d, 1H).

### Intermediate 012

### 5-Bromo-2-[3-(trifluoromethyl)phenoxy]pyridine-3-sulfonamide

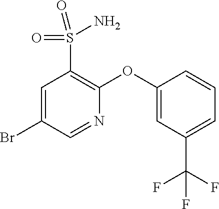 [SMILES: NS(=O)(=O)c1cc(Br)cnc1Oc1cccc(C(F)(F)F)c1]

3-Trifluoromethylphenol (475 mg, 2.93 mmol) was added to sodium hydroxide (1.17 g, 2.93 mmol) in water (1 mL). After 30 min water was removed under reduced pressure and the resulting alcoholate was added to a solution of 5-bromo-2-hydroxypyridine-3-sulfonamide (795 mg, 2.93 mmol) in acetonitrile (10 mL), together with potassium carbonate (1.21 g, 8.78 mmol), cesium carbonate (954 mg, 2.93 mmol) and 3-trifluoromethylphenol (475 mg, 2.93 mmol). The reaction mixture was stirred overnight at 110° C., cooled to room temperature and the solvent was removed under reduced pressure. Water and dichloromethane were added for extraction, the phases were separated, the organic phase was dried and concentrated in vacuo. Column chromatography on a Biotage Isolera system (silica gel, gradient n-hexane/ethyl acetate) led to 5-bromo-2-[3-(trifluoromethyl)phenoxy]pyridine-3-sulfonamide (290 mg, 0.73 mmol, 25% yield, 95% purity).

LC-MS (Method A): Rt=1.22 min

MS (ESIpos): m/z=397/399 (M+H)⁺

¹H NMR (400 MHz, DICHLOROMETHANE-d₂) δ [ppm]: 5.34 (s, 2H), 7.40-7.47 (m, 1H), 7.51 (s, 1H), 7.57-7.66 (m, 2H), 8.35 (d, 1H), 8.45 (d, 1H).

### Intermediate 013

### 5-Amino-2-[3-(trifluoromethyl)phenoxy]pyridine-3-sulfonamide

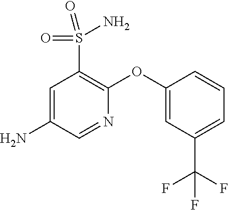 [SMILES: Nc1cnc(Oc2cccc(C(F)(F)F)c2)c(S(N)(=O)=O)c1]

5-Bromo-2-[3-(trifluoromethyl)phenoxy]pyridine-3-sulfonamide (280 mg, 0.705 mmol) was added to a solution of 1,1-dimethoxy-N,N-dimethylmethanamine (168 mg, 1.41 mmol) in dimethylformamide (5 mL), followed by stirring at room temperature for 1 h. The solvent was removed in vacuo, it was extracted with ethyl acetate and water, the organic phase was dried and concentrated in vacuo to yield crude 5-bromo-N-[(dimethylamino)methylene]-2-[3-(trifluoromethyl)phenoxy]pyridine-3-sulfonamide.

Crude 5-bromo-N-[(dimethylamino)methylene]-2-[3-(trifluoromethyl)phenoxy]pyridine-3-sulfonamide from the previous step was redissolved in dioxane (5 mL), the flask was flushed with argon, followed by addition of Xantphos (38.4 mg, 0.0663 mmol), palladium(II)acetate (7.45 mg, 0.0332 mmol), cesium carbonate (648 mg, 1.99 mmol) and 1,1-diphenylmethanimine (180 mg, 0.995 mmol). The reaction mixture was flushed again with argon, followed by stirring at 95° C. overnight. It was cooled to room temperature, the solvent was removed under reduced pressure and the resulting residue was extracted with water and ethyl acetate. The organic phase was dried over sodium sulfate and the solvent was removed under reduced pressure to obtain crude N-[(dimethylamino)methylene]-5-[(diphenylmethylene)amino]-2-[3-(trifluoromethyl)phenoxy]pyridine-3-sulfonamide.

Crude N-[(dimethylamino)methylene]-5-[(diphenylmethylene)amino]-2-[3-(trifluoromethyl)phenoxy]pyridine-3-sulfonamide was redissolved in ethanol (20 mL) and treated with 4N—HCl in dioxane (165 μL, 662 mmol), followed by stirring at room temperature for 1 h. Solvent was removed under reduced pressure to obtain crude 5-amino-2-[3-(trifluoromethyl)phenoxy]pyridine-3-sulfonamide (300 mg crude material).

LC-MS (Method A): Rt=0.97 min

MS (ESIpos): m/z=334 (M+H)⁺

### Intermediate 014

### N-(2,4-Dimethoxybenzyl)-5-nitro-2-{[2-(trifluoromethyl)pyrimidin-5-yl]oxy}benzenesulfonamide

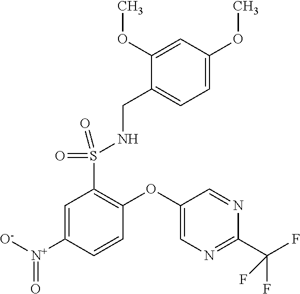 [SMILES: COc1ccc(CNS(=O)(=O)c2cc([N+](=O)[O-])ccc2Oc2cnc(C(F)(F)F)nc2)c(OC)c1]

According to general procedure GP1.1 2-chloro-N-(2,4-dimethoxybenzyl)-5-nitrobenzenesulfonamide (96.7 mg, 0.25 mmol) and 2-(trifluoromethyl)pyrimidin-5-ol (41 mg, 0.25 mmol) were converted to N-(2,4-dimethoxybenzyl)-5-nitro-2-{[2-(trifluoromethyl)pyrimidin-5-yl]oxy}benzenesulfonamide and were purified by column chromatography on a Biotage Isolera system (silica gel, gradient dichloromethane to dichloromethane/methanol 80/20) (80 mg, 0.156 mmol, 62% yield, 98% purity).

LC-MS (Method A): Rt=1.27 min

MS (ESIpos): m/z=515 (M+H)⁺

¹H-NMR (400 MHz, DMSO-d₆) δ [ppm]: 3.55 (s, 3H), 3.68 (s, 3H), 4.85 (s, 2H), 6.33 (d, 1H), 6.43 (dd, 1H), 7.11 (d, 1H), 7.19 (d, 1H), 8.32-8.37 (m, 2H), 9.06 (s, 2H).

### Intermediate 015

### 2-(2-Chlorophenyl)-N-(3-[(2,4-dimethoxybenzyl)sulfamoyl]-4-{[2-(trifluoromethyl)pyrimidin-5-yl]oxy}phenyl)acetamide

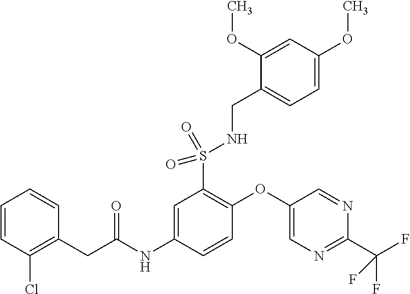 [SMILES: COc1ccc(CNS(=O)(=O)c2cc(NC(=O)Cc3ccccc3Cl)ccc2Oc2cnc(C(F)(F)F)nc2)c(OC)c1]

According to general procedures GP2.2 and GP3.2 purified N-(2,4-dimethoxybenzyl)-5-nitro-2-{[2-(trifluoromethyl)pyrimidin-5-yl]oxy}benzenesulfonamide (77.2 mg, 0.15 mmol) and (2-chlorophenyl)acetic acid (38.4 mg, 0.23 mmol) were converted without purification of intermediates to 2-(2-chlorophenyl)-N-(3-[(2,4-dimethoxybenzyl)sulfamoyl]-4-{[2-(trifluoromethyl)pyrimidin-5-yl]oxy}phenyl)acetamide and were purified by column chromatography on a Biotage Isolera system (silica gel, gradient dichloromethane/ethyl acetate) (35 mg, 0.0549 mmol, 37% yield, 98% purity).

LC-MS (Method A): Rt=1.34 min

MS (ESIpos): m/z=637 (M+H)⁺

¹H-NMR (400 MHz, DMSO-d₆) δ [ppm]: 3.58 (s, 3H), 3.69 (s, 3H), 3.79 (s, 2H), 4.84 (s, 2H), 6.41-6.45 (m, 2H), 6.96 (d, 1H), 7.13 (d, 1H), 7.26-7.32 (m, 2H), 7.38-7.46 (m, 2H), 7.69 (dd, 1H), 8.05 (d, 1H), 8.91 (s, 2H), 10.25 (s, 1H), 10.78 (s, 1H).

### Intermediate 016

### 2-(2-Chlorophenyl)-N-{3-[(2,4-dimethoxybenzyl)sulfamoyl]-4-[(2-isopropylpyrimidin-5-yl)oxy]phenyl}acetamide

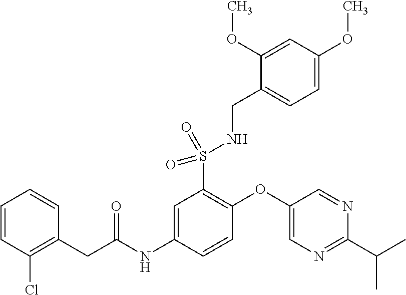 [SMILES: COc1ccc(CNS(=O)(=O)c2cc(NC(=O)Cc3ccccc3Cl)ccc2Oc2cnc(C(C)C)nc2)c(OC)c1]

According to general procedures GP1.1, GP2.2 and GP3.2 2-chloro-N-(2,4-dimethoxybenzyl)-5-nitrobenzenesulfonamide (96.7 mg, 0.25 mmol), 2-isopropylpyrimidin-5-ol (34.5 mg, 0.25 mmol) and (2-chlorophenyl)acetic acid (64.0 mg, 0.38 mmol) were converted without purification of intermediates to 2-(2-chlorophenyl)-N-{3-[(2,4-dimethoxybenzyl)sulfamoyl]-4-[(2-isopropylpyrimidin-5-yl)oxy]phenyl}acetamide and were purified at the end by column chromatography on a Biotage Isolera system (silica gel, gradient dichloromethane/ethyl acetate) (50 mg, 0.0818 mmol, 33% yield over 3 steps, 90% purity).

LC-MS (Method A): Rt=1.35 min

MS (ESIpos): m/z=611 (M+H)⁺

### Intermediate 017

### 2-(2-Chlorophenyl)-N-{4-[(2-cyclopropyl-4-methylpyrimidin-5-yl)oxy]-3-[(2,4-dimethoxybenzyl)sulfamoyl]phenyl}acetamide

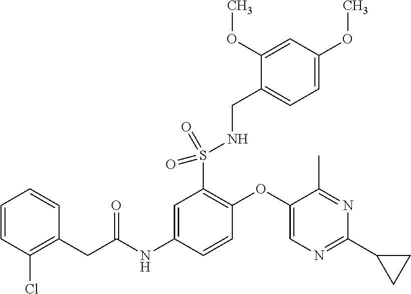 [SMILES: COc1ccc(CNS(=O)(=O)c2cc(NC(=O)Cc3ccccc3Cl)ccc2Oc2cnc(C3CC3)nc2C)c(OC)c1]

According to general procedures GP1.1, GP2.2 and GP3.2 2-chloro-N-(2,4-dimethoxybenzyl)-5-nitrobenzenesulfonamide (96.7 mg, 0.25 mmol), 2-cyclopropyl-4-methylpyrimidin-5-ol (37.5 mg, 0.25 mmol) and (2-chlorophenyl)acetic acid (64.0 mg, 0.38 mmol) were converted without purification of intermediates to 2-(2-chlorophenyl)-N-{4-[(2-cyclopropyl-4-methylpyrimidin-5-yl)oxy]-3-[(2,4-dimethoxybenzyl)sulfamoyl]phenyl}acetamide and were purified at the end by column chromatography on a Biotage Isolera system (silica gel, gradient dichloromethane/ethyl acetate) (50 mg, 0.0802 mmol, 32% yield over 3 steps, 90% purity).

LC-MS (Method A): Rt=1.35 min

MS (ESIpos): m/z=623 (M+H)⁺

### Intermediate 018

### N-{4-(4-Bromophenoxy)-3-[(2,4-dimethoxybenzyl)sulfamoyl]phenyl}-2-(2-chlorophenyl)acetamide

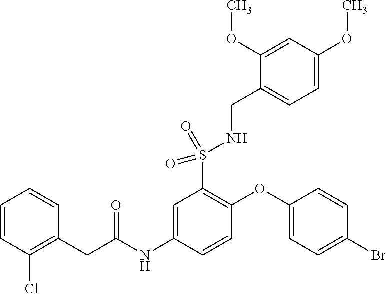 [SMILES: COc1ccc(CNS(=O)(=O)c2cc(NC(=O)Cc3ccccc3Cl)ccc2Oc2ccc(Br)cc2)c(OC)c1]

According to general procedures GP1.1, GP2.2 and GP3.2 2-chloro-N-(2,4-dimethoxybenzyl)-5-nitrobenzenesulfonamide (2.90 g, 7.50 mmol), 4-bromophenol (1.30 g, 7.50 mmol) and (2-chlorophenyl)acetic acid (1.15 g, 6.75 mmol) were converted without purification of intermediates to N-{4-(4-bromophenoxy)-3-[(2,4-dimethoxybenzyl)sulfamoyl]phenyl}-2-(2-chlorophenyl)acetamide. A small amount was purified at the end by column chromatography on a Biotage Isolera system (silica gel, gradient dichloromethane/ethyl acetate) for NMR characterization, the rest was used in the next step without further purification (purity 40%).

LC-MS (Method A): Rt=1.45 min

MS (ESIneg): m/z=645 (M−H)⁺

¹H-NMR (400 MHz, DMSO-d₆) δ [ppm]: 3.65 (s, 3H), 3.71 (s, 3H), 3.85 (s, 2H), 4.03 (d, 2H), 6.39-6.43 (m, 2H), 6.89-6.94 (m, 2H), 6.96 (d, 1H), 7.10-7.14 (m, 1H), 7.29-7.36 (m, 2H), 7.42-7.49 (m, 2H), 7.52-7.58 (m, 2H), 7.73 (t, 1H), 7.77 (dd, 1H), 8.12 (d, 1H), 10.50 (s, 1H).

### Intermediate 019

### N-{4-(3-Bromophenoxy)-3-[(2,4-dimethoxybenzyl)sulfamoyl]phenyl}-2-(2-chlorophenyl)acetamide

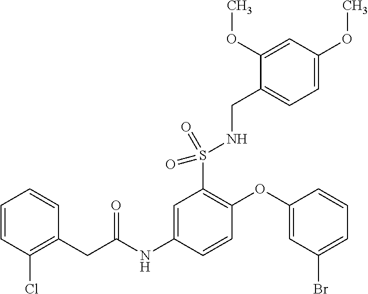 [SMILES: COc1ccc(CNS(=O)(=O)c2cc(NC(=O)Cc3ccccc3Cl)ccc2Oc2cccc(Br)c2)c(OC)c1]

According to general procedures GP1.1, GP2.2 and GP3.2 2-chloro-N-(2,4-dimethoxybenzyl)-5-nitrobenzenesulfonamide (2.90 g, 7.50 mmol), 3-bromophenol (1.30 g, 7.50 mmol) and (2-chlorophenyl)acetic acid (1.28 g, 7.50 mmol) were converted without purification of intermediates to N-{4-(3-bromophenoxy)-3-[(2,4-dimethoxybenzyl)sulfamoyl]phenyl}-2-(2-chlorophenyl)acetamide. A small amount was purified at the end by column chromatography on a Biotage Isolera system (silica gel, gradient dichloromethane/ethyl acetate) for NMR characterization, the rest was used in the next step without further purification (purity 40%).

LC-MS (Method A): Rt=1.43 min

MS (ESIneg): m/z=645 (M−H)⁺

¹H-NMR (400 MHz, DMSO-d₆) δ [ppm]: 3.66 (s, 3H), 3.71 (s, 3H), 3.86 (s, 2H), 4.03 (d, 2H), 6.38-6.42 (m, 2H), 6.92-6.97 (m, 1H), 7.01 (d, 1H), 7.10-7.13 (m, 1H), 7.14-7.16 (m, 1H), 7.29-7.37 (m, 4H), 7.42-7.49 (m, 2H), 7.75 (t, 1H), 7.79 (dd, 1H), 8.13 (d, 1H), 10.52 (s, 1H).

### Intermediate 020

### Methyl 3-{2-[(2,4-dimethoxybenzyl)sulfamoyl]-4-nitrophenoxy}benzoate

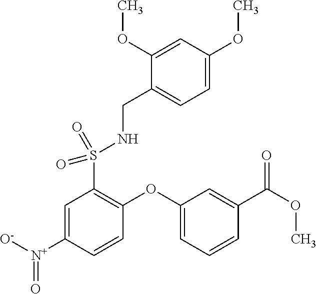 [SMILES: COC(=O)c1cccc(Oc2ccc([N+](=O)[O-])cc2S(=O)(=O)NCc2ccc(OC)cc2OC)c1]

2-Chloro-N-(2,4-dimethoxybenzyl)-5-nitrobenzenesulfonamide (484 mg, 1.25 mmol) was dissolved in acetonitrile (17.5 mL), cesium carbonate (407 mg, 1.25 mmol) and methyl 3-hydroxybenzoate (190 mg, 1.25 mmol) were added. The reaction mixture was stirred in a sealed vial overnight at 110° C. After cooling to room temperature the solvent was removed under reduced pressure and the crude was treated with dichloromethane and brine solution, the organic phase was separated, dried over sodium sulfate and concentrated in vacuo. Chromatography on a Biotage Isolera System (silica gel, gradient ethyl acetate to dichloromethane/methanol) led to methyl 3-{2-[(2,4-dimethoxybenzyl)sulfamoyl]-4-nitrophenoxy}benzoate (350 mg, 0.697 mmol, 56% yield, 98% purity).

LC-MS (Method A): Rt=1.32 min

MS (ESIneg): m/z=501 (M−H)⁺

### Intermediate 021

### Methyl 2-{2-[(2,4-dimethoxybenzyl)sulfamoyl]-4-nitrophenoxy}benzoate

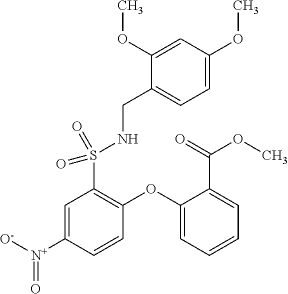 [SMILES: COC(=O)c1ccccc1Oc1ccc([N+](=O)[O-])cc1S(=O)(=O)NCc1ccc(OC)cc1OC]

2-Chloro-N-(2,4-dimethoxybenzyl)-5-nitrobenzenesulfonamide (484 mg, 1.25 mmol) was dissolved in acetonitrile (17.5 mL), cesium carbonate (407 mg, 1.25 mmol) and methyl 3-hydroxybenzoate (190 mg, 1.25 mmol) were added. It was stirred in a sealed vial overnight at 110° C. After cooling to room temperature the solvent was removed under reduced pressure and the crude was treated with dichloromethane and brine solution, the organic phase was separated, dried over sodium sulfate and concentrated in vacuo. Chromatography on a Biotage Isolera System (silica gel, gradient ethyl acetate to dichloromethane/methanol) led to methyl 2-{2-[(2,4-dimethoxybenzyl)sulfamoyl]-4-nitrophenoxy}benzoate (350 mg, 0.697 mmol, 56% yield, 98% purity).

LC-MS (Method A): Rt=1.34 min

MS (ESIneg): m/z=501 (M−H)⁺

### Intermediate 022

### Methyl 3-(4-{[(2-chlorophenyl)acetyl]amino}-2-[(2,4-dimethoxybenzyl)sulfamoyl]phenoxy)benzoate

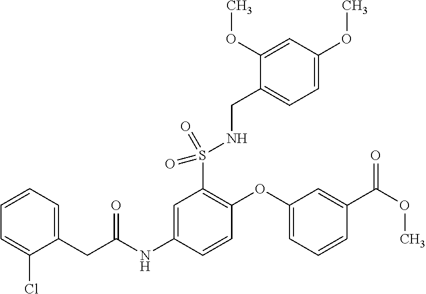 [SMILES: COC(=O)c1cccc(Oc2ccc(NC(=O)Cc3ccccc3Cl)cc2S(=O)(=O)NCc2ccc(OC)cc2OC)c1]

Methyl 3-{2-[(2,4-dimethoxybenzyl)sulfamoyl]-4-nitrophenoxy}benzoate (350 mg, 0.70 mmol) was dissolved in dioxane (5 mL) and treated with tin(II)chloride dihydrate (786 mg, 3.48 mmol). The reaction mixture was stirred in a sealed vial at 70° C. for 3 h, cooled to room temperature and filtered over a PTFE membrane. The filtrate was concentrated and redissolved in tetrahydrofuran (14 mL). (2-Chlorophenyl)acetic acid (179 mg, 1.05 mmol), N,N-diisopropylethylamine (1.36 g, 10.5 mmol) and HATU (399 mg, 1.05 mmol) were added and it was stirred overnight at room temperature. It was concentrated in vacuo and the crude was extracted and washed with water and dichloromethane. The organic phase was separated, dried over sodium sulfate and concentrated in vacuo. Chromatography on a Biotage Isolera system (silica gel, gradient dichloromethane/ethyl acetate) led to methyl 3-(4-{[(2-chlorophenyl)acetyl]amino}-2-[(2,4-dimethoxybenzyl)sulfamoyl]phenoxy)benzoate (150 mg, 0.240 mmol, 34% yield, 98% purity).

LC-MS (Method A): Rt=1.34 min

MS (ESIneg): m/z=623 (M−H)⁺

### Intermediate 023

### Methyl 2-(4-{[(2-chlorophenyl)acetyl]amino}-2-[(2,4-dimethoxybenzyl)sulfamoyl]phenoxy)benzoate

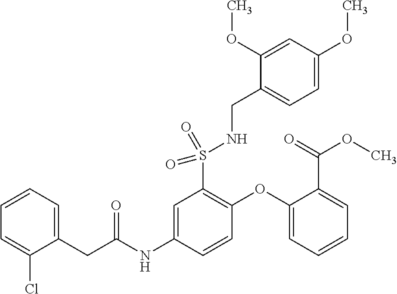 [SMILES: COC(=O)c1ccccc1Oc1ccc(NC(=O)Cc2ccccc2Cl)cc1S(=O)(=O)NCc1ccc(OC)cc1OC]

Methyl 2-{2-[(2,4-dimethoxybenzyl)sulfamoyl]-4-nitrophenoxy}benzoate (350 mg, 0.70 mmol) was dissolved in dioxane (5 mL) and treated with tin(II)chloride dihydrate (786 mg, 3.48 mmol). The reaction mixture was stirred in a sealed vial at 70° C. for 3 h, cooled to room temperature and filtered over a PTFE membrane. The filtrate was concentrated and redissolved in tetrahydrofuran (14 mL). (2-Chlorophenyl)acetic acid (179 mg, 1.05 mmol), N,N-diisopropylethylamine (1.36 g, 10.5 mmol) and HATU (399 mg, 1.05 mmol) were added and it was stirred overnight at room temperature. The solvent was removed in vacuo and the residue was extracted and washed with water and dichloromethane. The organic phase was separated, dried over sodium sulfate and concentrated in vacuo. Chromatography on a Biotage Isolera system (silica gel, gradient dichloromethane/ethyl acetate) led to methyl 2-(4-{[(2-chlorophenyl)acetyl]amino}-2-[(2,4-dimethoxybenzyl)sulfamoyl]phenoxy)benzoate (100 mg, 0.160 mmol, 23% yield, 98% purity).

LC-MS (Method A): Rt=1.36 min

MS (pos): m/z=625 (M+H)⁺

### Intermediate 024

### 2-(2-Chlorophenyl)-N-{3-[(2,4-dimethoxybenzyl)sulfamoyl]-4-[3-(2-hydroxypropan-2-yl)phenoxy]phenyl}acetamide

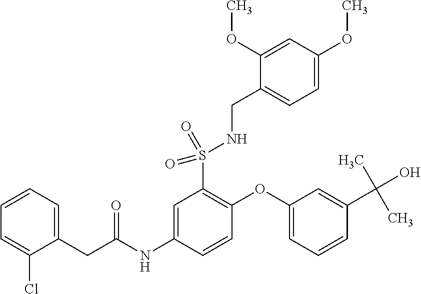 [SMILES: COc1ccc(CNS(=O)(=O)c2cc(NC(=O)Cc3ccccc3Cl)ccc2Oc2cccc(C(C)(C)O)c2)c(OC)c1]

To a solution of methyl 3-(4-{[(2-chlorophenyl)acetyl]amino}-2-[(2,4-dimethoxybenzyl)sulfamoyl]phenoxy)benzoate (125 mg, 0.20 mmol) in tetrahydrofuran (20 mL) was added at 0° C. methyl magnesium bromide solution (4.29 mL of 1.4 M in THF/toluene, 6.0 mmol). Stirring was continued at room temperature for 5 days. It was quenched with ammonium chloride solution, the solvent was removed under reduced pressure followed by extraction with water and dichloromethane. The organic phase was separated, dried over sodium sulfate and concentrated in vacuo. Chromatography on a Biotage Isolera system (silica gel, gradient dichloromethane/ethyl acetate) led to 2-(2-chlorophenyl)-N-{3-[(2,4-dimethoxybenzyl)sulfamoyl]-4-[3-(2-hydroxypropan-2-yl)phenoxy]phenyl}acetamide (45 mg, 0.0720 mmol, 36% yield, 98% purity).

LC-MS (Method A): Rt=1.28 min

MS (ESIneg): m/z=623 (M−H)⁺

¹H-NMR (400 MHz, DMSO-d₆) δ [ppm]: 1.41 (s, 6H), 3.66 (s, 3H), 3.71 (s, 3H), 3.84 (s, 2H), 4.05 (d, 2H), 5.09 (s, 1H), 5.77 (s, 1H), 6.39-6.43 (m, 2H), 6.76 (ddd, 1H), 6.84 (d, 1H), 7.12-7.35 (m, 5H), 7.41-7.48 (m, 2H), 7.64 (t, 1H), 7.74 (dd, 1H), 8.12 (d, 1H), 10.46 (s, 1H).

### Intermediate 025

### 2-(2-Chlorophenyl)-N-{3-[(2,4-dimethoxybenzyl)sulfamoyl]-4-[2-(2-hydroxypropan-2-yl)phenoxy]phenyl}acetamide

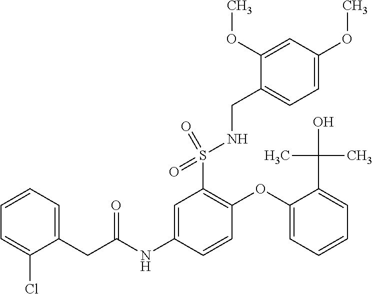 [SMILES: COc1ccc(CNS(=O)(=O)c2cc(NC(=O)Cc3ccccc3Cl)ccc2Oc2ccccc2C(C)(C)O)c(OC)c1]

To a solution of methyl 2-(4-{[(2-chlorophenyl)acetyl]amino}-2-[(2,4-dimethoxybenzyl)sulfamoyl]phenoxy)benzoate (68.8 mg, 0.11 mmol) in tetrahydrofuran (11 mL) was added at 0° C. methyl magnesium bromide solution (2.36 mL of 1.4 M in THF/toluene, 3.3 mmol). Stirring was continued at room temperature for 5 days. It was quenched with ammonium chloride solution, the solvent was removed under reduced pressure followed by extraction with water and dichloromethane. The organic phase was separated, dried over sodium sulfate and concentrated under reduced pressure. Chromatography on a Biotage Isolera system (silica gel, gradient dichloromethane/ethyl acetate) led to 2-(2-chlorophenyl)-N-{3-[(2,4-dimethoxybenzyl)sulfamoyl]-4-[2-(2-hydroxypropan-2-yl)phenoxy]phenyl}acetamide (35 mg, 0.0563 mmol, 51% yield, 98% purity).

LC-MS (Method A): Rt=1.32 min

MS (ESIneg): m/z=623 (M−H)⁺

¹H-NMR (400 MHz, DMSO-d₆) δ [ppm]: 1.48 (s, 6H), 3.59 (s, 3H), 3.73 (s, 3H), 3.85 (s, 2H), 4.00-4.08 (m, 2H), 5.18 (s, 1H), 5.77 (s, 1H), 6.41-6.46 (m, 2H), 6.61 (dd, 1H), 6.74 (d, 1H), 7.12-7.23 (m, 3H), 7.29-7.36 (m, 2H), 7.45 (s, 2H), 7.65 (t, 1H), 7.69-7.75 (m, 2H), 8.18 (d, 1H), 10.47 (s, 1H).

### Intermediate 026

### 2-(4-Chlorophenoxy)-N-(2,4-dimethoxybenzyl)-5-nitrobenzenesulfonamide

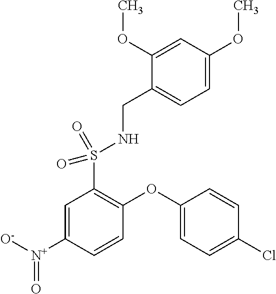 [SMILES: COc1ccc(CNS(=O)(=O)c2cc([N+](=O)[O-])ccc2Oc2ccc(Cl)cc2)c(OC)c1]

2-Chloro-N-(2,4-dimethoxybenzyl)-5-nitrobenzenesulfonamide (6.0 g, 16 mmol) was dissolved in acetonitrile (60 mL) and cesium carbonate (7.6 g, 23 mmol) and 4-chlorophenol (3.0 g, 23 mmol, 1.5 eq) were added. Stirring was continued at 110° C. until TLC showed consumption of starting material. After cooling to room temperature, the reaction mixture was filtered and the solvent was removed under reduced pressure. Afterwards water and ethyl acetate were added and the phases were separated. The organic phase was dried and the solvent was removed under reduced pressure. The crude was used without further purification.

### Intermediate 027

### 5-Amino-2-(4-chlorophenoxy)-N-(2,4-dimethoxybenzyl)benzenesulfonamide

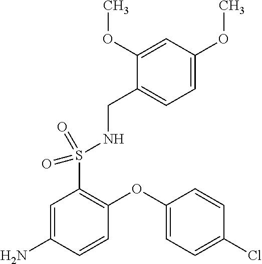 [SMILES: COc1ccc(CNS(=O)(=O)c2cc(N)ccc2Oc2ccc(Cl)cc2)c(OC)c1]

According to GP2.2 2-(4-chlorophenoxy)-N-(2,4-dimethoxybenzyl)-5-nitrobenzenesulfonamide (8.4 g, 5.2 mmol) was converted to 5-amino-2-(4-chlorophenoxy)-N-(2,4-dimethoxybenzyl)benzenesulfonamide and purified via column chromatography on a Biotage Isolera system (silica gel, gradient n-hexane/ethyl acetate)(2.8 g, 8.3 mmol, 40% yield).

### Intermediate 028

### 2-Chloro-5-nitrobenzenesulfonamide

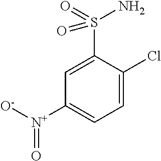 [SMILES: NS(=O)(=O)c1cc([N+](=O)[O-])ccc1Cl]

2-Chloro-5-nitrobenzenesulfonyl chloride (5.0 g, 20 mmol) was dissolved in dioxane (100 mL). Pyridine (7.0 g, 98 mmol, 7.9 mL) and ammonia (33% solution in dioxane, 39 mmol, 2.3 mL) were added. The reaction was stirred at 50° C. until completion of the reaction. After cooling to room temperature, the solvents were removed under reduced pressure and water was added. The suspension was filtered and the solid was dried and used without further purification. (3.6 g, 15 mmol, 78% yield)

### Intermediate 029

### 2-(Cyclobutyloxy)-5-nitrobenzenesulfonamide

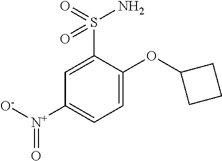 [SMILES: NS(=O)(=O)c1cc([N+](=O)[O-])ccc1OC1CCC1]

According to GP1.2 2-chloro-5-nitrobenzenesulfonamide (500 mg, 2.1 mmol) was reacted with cyclobutanol (229 mg, 3.2 mmol) and sodium hydride (0.6 g, 15 mmol, 60% purity). The crude was purified by column chromatography on a Biotage Isolera (silica gel, gradient n-hexanelethyl acetate) to yield pure 2-(cyclohexyloxy)-5-nitrobenzenesulfonamide (670 mg, 2.5 mmol, 116% yield).

### Intermediate 030

### 5-Amino-2-(cyclobutyloxy)benzenesulfonamide

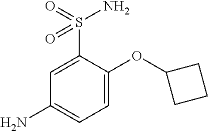 [SMILES: Nc1ccc(OC2CCC2)c(S(N)(=O)=O)c1]

According to GP 2.1 2-(cyclohexyloxy)-5-nitrobenzenesulfonamide (670 mg, 2.5 mmol) was converted to 5-amino-2-(cyclobutyloxy)benzenesulfonamide (470 mg, 1.9 mmol, 79% yield) and used in the next step without further purification.

### Intermediate 031

### 2-(Cyclohexyloxy)-5-nitrobenzenesulfonamide

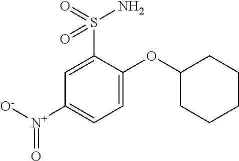 [SMILES: NS(=O)(=O)c1cc([N+](=O)[O-])ccc1OC1CCCCC1]

According to GP1.2 2-chloro-5-nitrobenzenesulfonamide (500 mg, 2.1 mmol) was reacted with cyclohexanol (254 mg, 2.5 mmol) and sodium hydride (0.3 g, 7.4 mmol, 60% purity). The crude was purified by column chromatography on a Biotage Isolera (silica gel, gradient n-hexane/ethyl acetate) to yield pure 2-(cyclohexyloxy)-5-nitrobenzenesulfonamide (430 mg, 1.43 mmol, 68% yield).

### Intermediate 032

### 5-Amino-2-(cyclohexyloxy)benzenesulfonamide

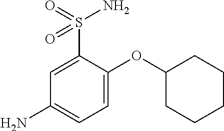 [SMILES: Nc1ccc(OC2CCCCC2)c(S(N)(=O)=O)c1]

According to GP2.1 2-(cyclohexyloxy)-5-nitrobenzenesulfonamide (430 mg, 1.43 mmol) was converted to 5-amino-2-(cyclohexyloxy)benzenesulfonamide (360 mg, 1.3 mmol, 93% yield) and used in the next step without further purification.

### Intermediate 033

### 5-Nitro-2-(tetrahydro-2H-pyran-4-yloxy)benzenesulfonamide

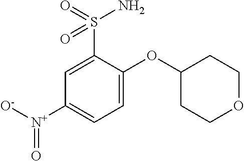 [SMILES: NS(=O)(=O)c1cc([N+](=O)[O-])ccc1OC1CCOCC1]

According to GP1.2 2-chloro-5-nitrobenzenesulfonamide (500 mg, 2.1 mmol) was reacted with tetrahydro-2H-pyran-4-ol (324 mg, 3.2 mmol) and sodium hydride (0.6 g, 15 mmol, 60% purity). The crude was purified by column chromatography on a Biotage Isolera (silica gel, gradient n-hexane/ethyl acetate) to yield pure 2-(cyclohexyloxy)-5-nitrobenzenesulfonamide (420 mg, 1.4 mmol, 66% yield).

### Intermediate 034

### 5-Amino-2-(tetrahydro-2H-pyran-4-yloxy)benzenesulfonamide

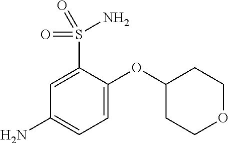 [SMILES: Nc1ccc(OC2CCOCC2)c(S(N)(=O)=O)c1]

According to GP2.1 2-(cyclohexyloxy)-5-nitrobenzenesulfonamide (420 mg, 1.4 mmol) was converted to 5-amino-2-(tetrahydro-2H-pyran-4-yloxy)benzenesulfonamide (420 mg, 1.5 mmol, quant. yield) and used in the next step without further purification.

### Intermediate 035

### tert-Butyl 3-{2-[(2,4-dimethoxybenzyl)sulfamoyl]-4-nitrophenoxy}azetidine-1-carboxylate

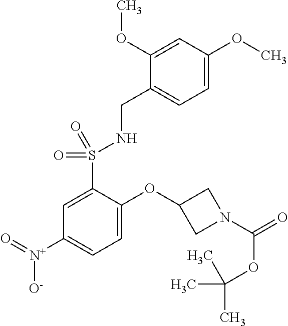 [SMILES: COc1ccc(CNS(=O)(=O)c2cc([N+](=O)[O-])ccc2OC2CN(C(=O)OC(C)(C)C)C2)c(OC)c1]

According to GP1.2 2-chloro-N-(2,4-dimethoxybenzyl)-5-nitrobenzenesulfonamide (500 mg, 1.3 mmol) was reacted with tert-butyl 3-hydroxyazetidine-1-carboxylate (336 mg, 1.9 mmol) and sodium hydride (217 mg, 9 mmol). The crude was purified by column chromatography on a Biotage Isolera (silica gel, gradient n-hexane/ethyl acetate) to yield pure tert-butyl 3-{2-[(2,4-dimethoxybenzyl)sulfamoyl]-4-nitrophenoxy}azetidine-1-carboxylate (510 mg, 1.0 mmol, 75% yield).

### Intermediate 036

### tert-Butyl 3-{4-amino-2-[(2,4-dimethoxybenzyl)sulfamoyl]phenoxy}azetidine-1-carboxylate

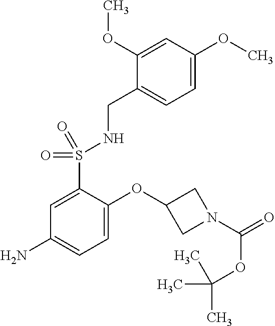 [SMILES: COc1ccc(CNS(=O)(=O)c2cc(N)ccc2OC2CN(C(=O)OC(C)(C)C)C2)c(OC)c1]

According to GP2.1 tert-butyl 3-{2-[(2,4-dimethoxybenzyl)sulfamoyl]-4-nitrophenoxy}azetidine-1-carboxylate (510 mg, 1.0 mmol) was converted to tert-butyl 3-{4-amino-2-[(2,4-dimethoxybenzyl)sulfamoyl]phenoxy}azetidine-1-carboxylate (490 mg, 1.0 mmol, 100% yield) and used in the next step without further purification.

### Intermediate 037

### 2-(Cyclopentyloxy)-N-(2,4-dimethoxybenzyl)-5-nitrobenzenesulfonamide

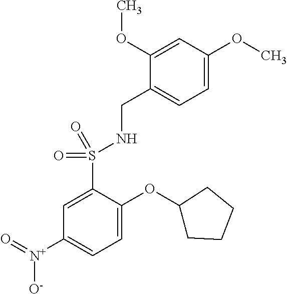 [SMILES: COc1ccc(CNS(=O)(=O)c2cc([N+](=O)[O-])ccc2OC2CCCC2)c(OC)c1]

According to GP1.2 2-chloro-N-(2,4-dimethoxybenzyl)-5-nitrobenzenesulfonamide (500 mg, 1.3 mmol) was reacted with cyclopentanol (334 mg, 3.9 mmol) and sodium hydride (310 mg, 13 mmol). The crude was purified by column chromatography on a Biotage Isolera (silica gel, gradient n-hexane/ethyl acetate) to yield pure 2-(cyclopentyloxy)-N-(2,4-dimethoxybenzyl)-5-nitrobenzenesulfonamide (470 mg, 1.0 mmol, 83% yield).

### Intermediate 038

### 5-Amino-2-(cyclopentyloxy)-N-(2,4-dimethoxybenzyl)benzenesulfonamide

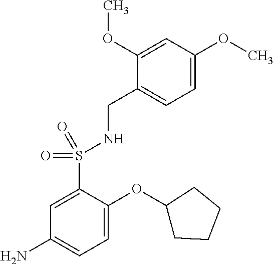 [SMILES: COc1ccc(CNS(=O)(=O)c2cc(N)ccc2OC2CCCC2)c(OC)c1]

According to GP1.2 2-(cyclopentyloxy)-N-(2,4-dimethoxybenzyl)-5-nitrobenzenesulfonamide (470 mg, 1.0 mmol) was converted to 5-amino-2-(cyclopentyloxy)-N-(2,4-dimethoxybenzyl)benzenesulfonamide (470 mg, 1.2 mmol, quant. yield) and used in the next step without further purification.

### Intermediate 039

### N-(2,4-Dimethoxybenzyl)-5-nitro-2-[(3S)-tetrahydrothiophen-3-yloxy]benzenesulfonamide

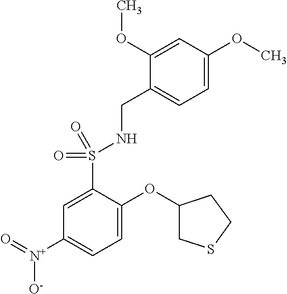 [SMILES: COc1ccc(CNS(=O)(=O)c2cc([N+](=O)[O-])ccc2OC2CCSC2)c(OC)c1]

According to GP1.2 2-chloro-N-(2,4-dimethoxybenzyl)-5-nitrobenzenesulfonamide (1.6 g, 4.2 mmol) was reacted with tetrahydrothiophene-3-ol (650 mg, 6.2 mmol) and sodium hydride (699 mg, 29 mmol). The crude was purified by column chromatography on a Biotage Isolera (silica gel, gradient n-hexane/ethyl acetate) to yield pure 2-(cyclopentyloxy)-N-(2,4-dimethoxybenzyl)-5-nitrobenzenesulfonamide (1.2 g, 2.6 mmol, 63% yield).

### Intermediate 040

### 2-(2-Chlorophenyl)-N-{3-[(2,4-dimethoxybenzyl)sulfamoyl]-4-[(3S)-tetrahydrothiophen-3-yloxy]phenyl}acetamide

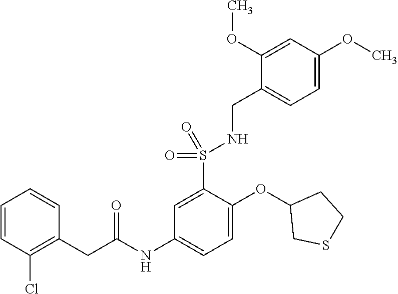 [SMILES: COc1ccc(CNS(=O)(=O)c2cc(NC(=O)Cc3ccccc3Cl)ccc2OC2CCSC2)c(OC)c1]

According to GP2.3 and GP3.2 2-(cyclopentyloxy)-N-(2,4-dimethoxybenzyl)-5-nitrobenzenesulfonamide (1.2 g, 2.6 mmol) was converted with (2-chlorophenyl)acetic acid (550 mg, 3.2 mmol) to 2-(2-chlorophenyl)-N-{3-[(2,4-dimethoxybenzyl)sulfamoyl]-4-[(3S)-tetrahydrothiophen-3-yloxy]phenyl}acetamide and purified by column chromatography on a Biotage Isolera (silica gel, gradient n-hexane/ethyl acetate) (1.7 g, 3.0 mmol, 110% yield).

### Intermediate 041

### 2-(2-Chlorophenyl)-N-(3-[(2,4-dimethoxybenzyl)sulfamoyl]-4-{[(3S)-1,1-dioxidotetrahydrothiophen-3-yl]oxy}phenyl)acetamide

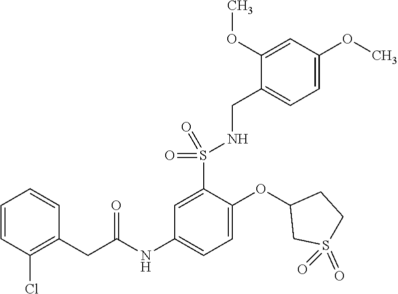 [SMILES: COc1ccc(CNS(=O)(=O)c2cc(NC(=O)Cc3ccccc3Cl)ccc2OC2CCS(=O)(=O)C2)c(OC)c1]

2-(2-Chlorophenyl)-N-{3-[(2,4-dimethoxybenzyl)sulfamoyl]-4-[(3S)-tetrahydrothiophen-3-yloxy]phenyl}acetamide (100 mg, 0.2 mmol) was dissolved in dichloromethane (2 mL) and 3-chlorobenzenecarboperoxoic acid (119 mg, 0.52 mmol, 75% purity) were added at room temperature. Stirring was continued for 16 h, afterwards, sat. aq. sodium bicarbonate and ethyl acetate were added. The phases were separated and the organic phase was dried. After removal of the solvent under reduced pressure, the crude was purified by preparative HPLC (Chromatorex C-18 10 μm, 125×30 mm, acetonitrile/water+0.1% formic acid) to yield 2-(2-chlorophenyl)-N-(3-[(2,4-dimethoxybenzyl)sulfamoyl]-4-{[(3S)-1,1-dioxidotetrahydrothiophen-3-yl]oxy}phenyl)acetamide (18 mg, 0.030 mmol, 17% yield).

LC-MS (Method A): Rt=min 1.17

MS (ESIpos): m/z=609 (M+H)⁺

### Intermediate 042

### N-(2,4-Dimethoxybenzyl)-2-{[(3R)-1-methylpyrrolidin-3-yl]oxy}-5-nitrobenzenesulfonamide

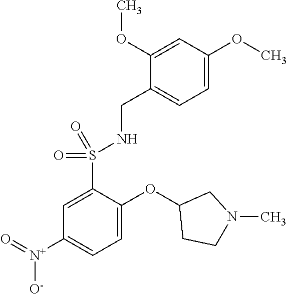 [SMILES: COc1ccc(CNS(=O)(=O)c2cc([N+](=O)[O-])ccc2OC2CCN(C)C2)c(OC)c1]

According to GP1.2 2-chloro-N-(2,4-dimethoxybenzyl)-5-nitrobenzenesulfonamide (500 mg, 1.3 mmol) was reacted with 1-methylpyrrolidin-3-ol (196 mg, 1.9 mmol) and sodium hydride (217 mg, 9.0 mmol). The crude was purified by column chromatography on a Biotage Isolera (silica gel, 2% gradient of ethanol in dichloromethane) to yield N-(2,4-dimethoxybenzyl)-2-{[(3R)-1-methylpyrrolidin-3-yl]oxy}-5-nitrobenzenesulfonamide (480 mg, 1.0 mmol, 82% yield).

### Intermediate 043

### 5-Amino-N-(2,4-dimethoxybenzyl)-2-{[(3R)-1-methylpyrrolidin-3-yl]oxy}benzenesulfonamide

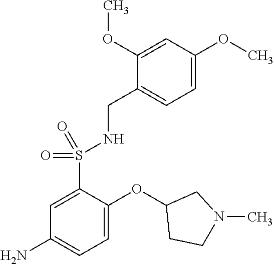 [SMILES: COc1ccc(CNS(=O)(=O)c2cc(N)ccc2OC2CCN(C)C2)c(OC)c1]

According to GP2.1 N-(2,4-dimethoxybenzyl)-2-{[(3R)-1-methylpyrrolidin-3-yl]oxy}-5-nitrobenzenesulfonamide (480 mg, 1.0 mmol) was converted to 5-amino-2-(cyclopentyloxy)-N-(2,4-dimethoxybenzyl)benzenesulfonamide (450 mg, 1.1 mmol, 100% yield) and used in the next step without further purification.

### Intermediate 044

### 5-Amino-N-(2,4-dimethoxybenzyl)-2-[(1-methylpiperidin-4-yl)oxy]benzenesulfonamide

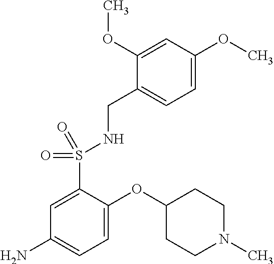 [SMILES: COc1ccc(CNS(=O)(=O)c2cc(N)ccc2OC2CCN(C)CC2)c(OC)c1]

According to GP1.2 and GP2.1 2-chloro-N-(2,4-dimethoxybenzyl)-5-nitrobenzenesulfonamide (500 mg, 1.3 mmol) was reacted with 1-methylpiperidin-4-ol (223 mg, 1.9 mmol) to yield 5-amino-N-(2,4-dimethoxybenzyl)-2-[(1-methylpiperidin-4-yl)oxy]benzenesulfonamide (780 mg, 1.8 mmol, 31% yield over 2 steps).

### Intermediate 045

### N-(2,4-Dimethoxybenzyl)-2-{[(3R)-1-methylpiperidin-3-yl]oxy}-5-nitrobenzenesulfonamide

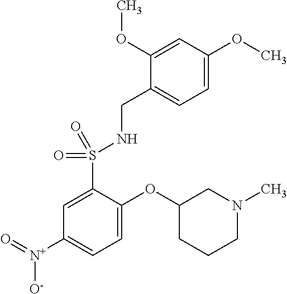 [SMILES: COc1ccc(CNS(=O)(=O)c2cc([N+](=O)[O-])ccc2OC2CCCN(C)C2)c(OC)c1]

According to GP1.2 2-chloro-N-(2,4-dimethoxybenzyl)-5-nitrobenzenesulfonamide (500 mg, 1.3 mmol) was reacted with 1-methylpiperidin-3-ol (223 mg, 1.9 mmol) and sodium hydride (217 mg, 9.0 mmol). The crude was purified by column chromatography on a Biotage Isolera (silica gel, 2% gradient of ethanol in dichloromethane) to yield N-(2,4-dimethoxybenzyl)-2-{[(3R)-1-methylpiperidin-3-yl]oxy}-5-nitrobenzenesulfonamide (470 mg, 1.0 mmol, 78% yield).

### Intermediate 046

### 5-Amino-N-(2,4-dimethoxybenzyl)-2-{[(3R)-1-methylpiperidin-3-yl]oxy}benzenesulfonamide

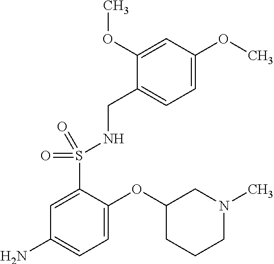 [SMILES: COc1ccc(CNS(=O)(=O)c2cc(N)ccc2OC2CCCN(C)C2)c(OC)c1]

According to GP2.1 N-(2,4-dimethoxybenzyl)-2-{[(3R)-1-methylpiperidin-3-yl]oxy}-5-nitrobenzenesulfonamide (470 mg, 1.0 mmol) was converted to 5-amino-N-(2,4-dimethoxybenzyl)-2-{[(3R)-1-methylpiperidin-3-yl]oxy}benzenesulfonamide (480 mg, 1.1 mmol, quant. yield) and used in the next step without further purification.

### Intermediate 047

### N-{4-(3-Chlorophenoxy)-3-[(2,4-dimethoxybenzyl)sulfamoyl]phenyl}-2-phenylacetamide

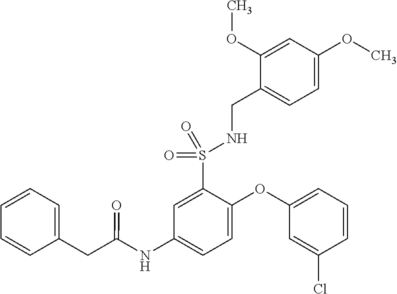 [SMILES: COc1ccc(CNS(=O)(=O)c2cc(NC(=O)Cc3ccccc3)ccc2Oc2cccc(Cl)c2)c(OC)c1]

5-Amino-2-(3-chlorophenoxy)-N-(2,4-dimethoxybenzyl)benzenesulfonamide (500 mg, 1.14 mmol) was converted according to GP3.2 to N-{4-(3-chlorophenoxy)-3-[(2,4-dimethoxybenzyl)sulfamoyl]phenyl}-2-phenylacetamide. The crude was purified by column chromatography on a Biotage Isolera system (silica gel, gradient n-hexane/ethyl acetate) (550 mg, 631 mmol, 87% yield, 99% purity).

LC-MS (Method A): Rt=min 1.39

MS (ESIpos): m/z=567 (M+H)⁺

¹H-NMR (400 MHz, DMSO-d₆) δ [ppm]: 3.54-3.73 (m, 8H), 4.01 (d, 2H), 6.30-6.42 (m, 2H), 6.80-6.92 (m, 1H), 6.92-7.03 (m, 2H), 7.04-7.15 (m, 1H), 7.15-7.23 (m, 1H), 7.23-7.31 (m, 1H), 7.31-7.39 (m, 5H), 7.63-7.75 (m, 1H), 7.75-7.85 (m, 1H), 8.08 (d, 1H), 10.44 (s, 1H).

### Intermediate 048

### N-{4-(3-Chlorophenoxy)-3-[(2,4-dimethoxybenzyl)sulfamoyl]phenyl}-2-(pyridin-2-yl)acetamide

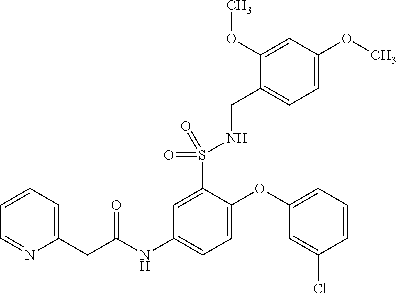 [SMILES: COc1ccc(CNS(=O)(=O)c2cc(NC(=O)Cc3ccccn3)ccc2Oc2cccc(Cl)c2)c(OC)c1]

5-Amino-2-(3-chlorophenoxy)-N-(2,4-dimethoxybenzyl)benzenesulfonamide (200 mg, 0.446 mmol) was converted according to GP3.2 to N-{4-(3-chlorophenoxy)-3-[(2,4-dimethoxybenzyl)sulfamoyl]phenyl}-2-(pyridin-2-yl)acetamide (quantitative yield).

### Intermediate 049

### N-{4-(3-Chlorophenoxy)-3-[(2,4-dimethoxybenzyl)sulfamoyl]phenyl}-2-(pyridin-3-yl)acetamide

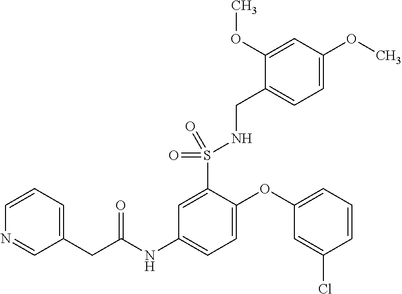 [SMILES: COc1ccc(CNS(=O)(=O)c2cc(NC(=O)Cc3cccnc3)ccc2Oc2cccc(Cl)c2)c(OC)c1]

5-Amino-2-(3-chlorophenoxy)-N-(2,4-dimethoxybenzyl)benzenesulfonamide (150 mg, 0.267 mmol) was converted according to GP3.2 to N-{4-(3-chlorophenoxy)-3-[(2,4-dimethoxybenzyl)sulfamoyl]phenyl}-2-(pyridin-3-yl)acetamide (quantitative yield).

### Intermediate 050

### N-{4-(3-Chlorophenoxy)-3-[(2,4-dimethoxybenzyl)sulfamoyl]phenyl}-2-(3-chlorophenyl)acetamide

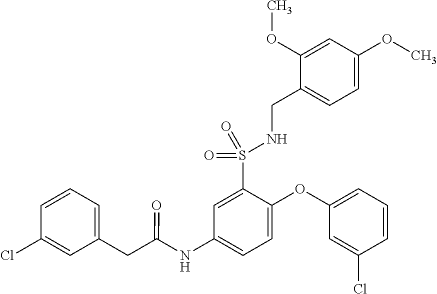 [SMILES: COc1ccc(CNS(=O)(=O)c2cc(NC(=O)Cc3cccc(Cl)c3)ccc2Oc2cccc(Cl)c2)c(OC)c1]

5-Amino-2-(3-chlorophenoxy)-N-(2,4-dimethoxybenzyl)benzenesulfonamide (170 mg, 0.284 mmol) was converted according to GP3.2 to N-{4-(3-chlorophenoxy)-3-[(2,4-dimethoxybenzyl)sulfamoyl]phenyl}-2-(3-chlorophenyl)acetamide. The crude was purified by preparative HPLC (Waters XBrigde C18 5μ 100×30 mm, acetonitrile/water+0.2% aqueous ammonia (32%)).

LC-MS (Method B): Rt=min 1.45

MS (ESIneg): m/z=599 (M−H)⁺

### Intermediate 051

### N-{4-(3-Chlorophenoxy)-3-[(2,4-dimethoxybenzyl)sulfamoyl]phenyl}-2-(2-chlorophenyl)acetamide

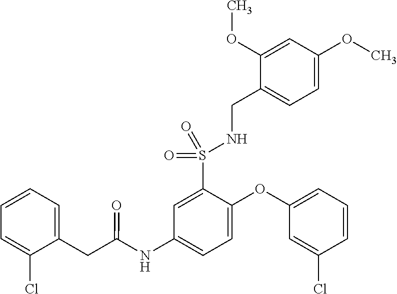 [SMILES: COc1ccc(CNS(=O)(=O)c2cc(NC(=O)Cc3ccccc3Cl)ccc2Oc2cccc(Cl)c2)c(OC)c1]

5-Amino-2-(3-chlorophenoxy)-N-(2,4-dimethoxybenzyl)benzenesulfonamide (170 mg, 0.284 mmol) were converted according to GP3.2 to N-{4-(3-chlorophenoxy)-3-[(2,4-dimethoxybenzyl)sulfamoyl]phenyl}-2-(2-chlorophenyl)acetamide. The crude was purified by preparative HPLC (Waters XBrigde C18 5μ 100×30 mm, acetonitrile/water+0.2% aqueous ammonia (32%)) (70 mg, 0.120 mmol, 42% yield).

LC-MS (Method E): Rt=1.29 min

MS (ESIneg): m/z=580 (M−H)⁺

### Intermediate 052

### N-{4-(3-Chlorophenoxy)-3-[(2,4-dimethoxybenzyl)sulfamoyl]phenyl}-2-(4-methoxyphenyl)acetamide

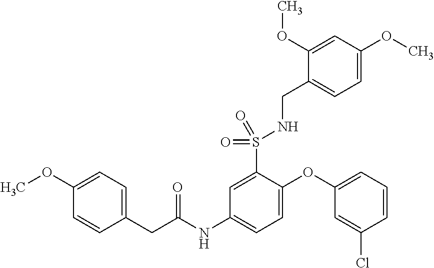

5-Amino-2-(3-chlorophenoxy)-N-(2,4-dimethoxybenzyl)benzenesulfonamide (175 mg, 0.292 mmol) was converted according to GP3.2 to N-{4-(3-chlorophenoxy)-3-[(2,4-dimethoxybenzyl)sulfamoyl]phenyl}-2-(4-methoxyphenyl)acetamide. The crude was purified by preparative HPLC (Waters XBrigde C18 5μ 100×30 mm, acetonitrile/water+0.2% aqueous ammonia (32%)) (70 mg, 0.120 mmol, 40% yield).

LC-MS (Method E): Rt=1.34 min

MS (ESIneg): m/z=595 (M−H)⁺

### Intermediate 053

### N-{4-(3-Chlorophenoxy)-3-[(2,4-dimethoxybenzyl)sulfamoyl]phenyl}-2-(3-methoxyphenyl)acetamide

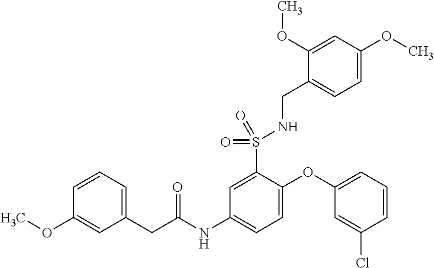

According to GP 3.1 5-amino-2-(3-chlorophenoxy)-N-(2,4-dimethoxybenzyl)benzenesulfonamide (175 mg, 0.29 mmol) was reacted with (3-methoxyphenyl)acetic acid (53 mg, 0.32 mmol). The crude was purified by preparative HPLC (Waters XBrigde C18 5μ 100×30 mm, acetonitrile/water+0.2% aqueous ammonia (32%)) to yield pure N-{4-(3-chlorophenoxy)-3-[(2,4-dimethoxybenzyl)sulfamoyl]phenyl}-2-(3-methoxyphenyl)acetamide (70 mg, 0.12 mmol, 40% yield).

LC-MS (Method E): Rt=1.34 min

MS (ESIneg): m/z=595 (M−H)⁺

### Intermediate 054

### N-{4-(3-Chlorophenoxy)-3-[(2,4-dimethoxybenzyl)sulfamoyl]phenyl}-2-(2-methoxyphenyl)acetamide

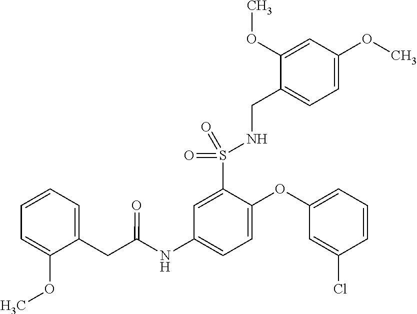 [SMILES: COc1ccc(CNS(=O)(=O)c2cc(NC(=O)Cc3ccccc3OC)ccc2Oc2cccc(Cl)c2)c(OC)c1]

According to GP 3.1 5-amino-2-(3-chlorophenoxy)-N-(2,4-dimethoxybenzyl)benzenesulfonamide (175 mg, 0.29 mmol) was reacted with (2-methoxyphenyl)acetic acid (53 mg, 0.32 mmol). The crude was purified by preparative HPLC (Waters XBrigde C18 5μ 100×30 mm, acetonitrile/water+0.2% aqueous ammonia (32%)) to yield pure N-{4-(3-chlorophenoxy)-3-[(2,4-dimethoxybenzyl)sulfamoyl]phenyl}-2-(3-methoxyphenyl)acetamide (70 mg, 0.12 mmol, 40% yield).

LC-MS (Method E): Rt=1.36 min

MS (ESIneg): m/z=595 (M−H)⁺

### Intermediate 055

### N-{4-(3-Chlorophenoxy)-3-[(2,4-dimethoxybenzyl)sulfamoyl]phenyl}-2-(5-methylpyridin-2-yl)acetamide

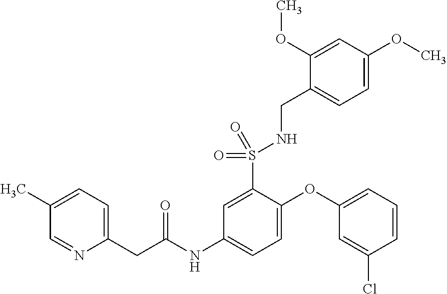

According to GP3.2 5-amino-2-(3-chlorophenoxy)-N-(2,4-dimethoxybenzyl)benzenesulfonamide (150 mg, 0.334 mmol) and (5-methylpyridin-2-yl)acetic acid (121 mg, 0.401 mmol) were reacted to N-{4-(3-chlorophenoxy)-3-[(2,4-dimethoxybenzyl)sulfamoyl]phenyl}-2-(5-methylpyridin-2-yl)acetamide. The crude was purified by preparative HPLC (Waters XBrigde C18 5μ 100×30 mm, acetonitrile/water+0.2% aqueous ammonia (32%)) (75 mg, 0.130 mmol, 45% yield).

LC-MS (Method E): Rt=1.29 min

MS (ESIneg): m/z=580 (M−H)⁺

### Intermediate 056

### N-{4-(3-Chlorophenoxy)-3-[(2,4-dimethoxybenzyl)sulfamoyl]phenyl}-2-(pyridin-4-yl)acetamide

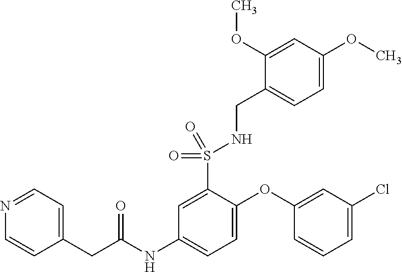 [SMILES: COc1ccc(CNS(=O)(=O)c2cc(NC(=O)Cc3ccncc3)ccc2Oc2cccc(Cl)c2)c(OC)c1]

According to GP3.2 5-amino-2-(3-chlorophenoxy)-N-(2,4-dimethoxybenzyl)benzenesulfonamide (170 mg, 0.284 mmol) and (pyridin-4-ylacetic acid (42 mg, 0.312 mmol) were reacted to N-{4-(3-chlorophenoxy)-3-[(2,4-dimethoxybenzyl)sulfamoyl]phenyl}-2-(5-methylpyridin-2-yl)acetamide. The crude was purified by preparative HPLC (Waters XBrigde C18 5μ 100×30 mm, acetonitrile/water+0.2% aqueous ammonia (32%)) (70 mg, 0.120 mmol, 43% yield).

LC-MS (Method B): Rt=min 1.22

MS (ESIneg): m/z=566 (M−H)⁺

### Intermediate 057

### N-(2,4-Dimethoxybenzyl)-2-fluoro-4-methyl-5-nitrobenzenesulfonamide

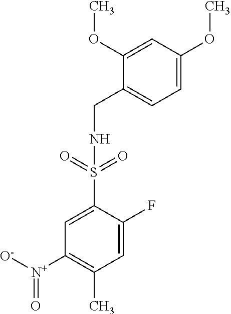 [SMILES: COc1ccc(CNS(=O)(=O)c2cc([N+](=O)[O-])c(C)cc2F)c(OC)c1]

To a solution of 2-fluoro-4-methyl-5-nitrobenzenesulfonyl chloride (1 g, 3.9 mmol) in dichloromethane (20 mL) was added sodium bicarbonate (1.0 g, 4.3 mmol) and 1-(2,4-dimethoxyphenyl)methanamine (0.7 g, 4.3 mmol) at 0° C. The mixture was stirred at room temperature overnight. Then all volatile components were removed in vacuo, followed by addition of water and ethyl acetate. After stirring for 10 min the resulting precipitate was separated by filtration and it was dried at 40° C. over night in vacuo to obtain N-(2,4-dimethoxybenzyl)-2-fluoro-4-methyl-5-nitrobenzenesulfonamide (1.5 g, 4.0 mmol, 100% yield). The intermediate was used in the next steps without further purification.

LC-MS (Method A): Rt=1.16 min

MS (ESIneg): m/z=383 (M−H)⁺

### Intermediate 058

### [2-(2-Methoxyethoxy)phenyl]acetic Acid

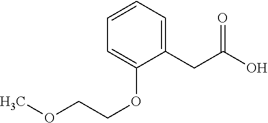

(2-Hydroxyphenyl)acetic acid (10 g, 66 mmol) was dissolved in dimethylformamide (100 mL) and bicarbonate (8.2 g, 98 mmol) was added. (Bromomethyl)benzene (12.4 g, 72 mmol) in dimethylformamide (5 mL) was added dropwise and stirring was continued for 18 h at room temperature. Water and ethyl acetate were added and the phases were separated. The organic phase was dried and the solvent removed under reduced pressure. The crude was recrystallized from n-hexane/methyl tert-butyl ether to yield benzyl (2-hydroxyphenyl)acetate (12.7 g, 52 mmol, 80% yield).

LC-MS (Method A): Rt=1.14 min

MS (ESIpos): m/z=243 (M+H)⁺

In the next step, benzyl (2-hydroxyphenyl)acetate (1 g, 4.1 mmol) and 2-methoxyethyl 4-methylbenzenesulfonate (2.4 g, 8.2 mmol, 80% purity) were dissolved in dimethylformamide (14 mL). Cesium carbonate (2.7 g, 8.3 mmol) was added and the reaction mixture was stirred for 2 days at 50° C. After cooling to room temperature, water and dichloromethane were added and the phases separated. The organic phase was dried and solvent was removed under reduced pressure. The crude was purified by column chromatography on a Biotage Isolera system (silica gel, gradient n-hexane/ethyl acetate) to yield benzyl [2-(2-methoxyethoxy)phenyl]acetate (621 mg, 1.3 mmol, 50% yield, 65% purity).

LC-MS (Method A): Rt=1.32 min

MS (ESIpos): m/z=301 (M+H)⁺

Benzyl [2-(2-methoxyethoxy)phenyl]acetate (621 mg, 1.3 mmol) was converted to [2-(2-methoxyethoxy)phenyl]acetic acid by GP2.1 and the crude was used without further purification in the next step.

LC-MS (Method A): Rt=0.79 min

MS (ESIpos): m/z=211 (M+H)⁺

### Intermediate 059

### {3-[(2-Methoxyethyl)(methyl)carbamoyl]phenyl}acetic Acid

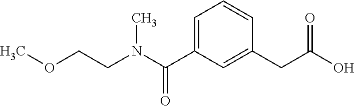 [SMILES: COCCN(C)C(=O)c1cccc(CC(=O)O)c1]

(3-Bromophenyl)acetic acid (5.0 g, 23 mmol) was dissolved in tetrahydrofurane (63 mL) and trifluoroacetic anhydride (12 g, 58 mmol) was added at 0° C. After 1 h, tert-butanol (22 g, 302 mmol) was added dropwise and the reaction was stirred at room temperature until TLC showed disappearance of starting material. The reaction was cooled to 0° C. and quenched by addition of saturated aqueous bicarbonate solution (100 mL). Ethyl acetate was added and the phases were separated. The organic phase was dried and concentrated in vacuo. The crude was used without further purification in the next step. tert-Butyl (3-bromophenyl)acetate (1 g, 3.7 mmol), 2-methoxy-N-methylethanamine (1 g, 11 mmol), tri-tert-butylphosphonium tetrafluoroborate (53 mg, 0.184 mmol)), tri-tert-butylphosphonium tetrafluoroborate (106 mg, 0.369 mmol), palladium(II) acetate (83 mg, 0.367 mmol), carbon monooxide-molybdenum (6:1) (1 g, 3.7 mmol) and sodium carbonate (1.2 g, 1.1 mmol) were dissolved in dioxane (29 mL) under argon atmosphere. Drops of water were added and the reaction was stirred at 100° C. for 18 h. After cooling to room temperature, the reaction mixture was filtered and concentrated. The crude was purified by column chromatography on silica gel on a Biotage Isolera system (silica gel, gradient n-hexane/ethyl acetate) to yield tert-butyl {3-[(2-methoxyethyl)(methyl)carbamoyl]phenyl}acetate (100 mg, 0.325 mmol, 9% yield).

LC-MS (Method A): Rt=1.14 min

MS (ESIpos): m/z=308 (M+H)⁺

tert-Butyl {3-[(2-methoxyethyl)(methyl)carbamoyl]phenyl}acetate (100 mg, 0.325 mmol) was converted according to GP4 to {3-[(2-methoxyethyl)(methyl)carbamoyl]phenyl}acetic acid (100 mg, 0.400 mmol). The crude was co-distilled twice with toluene and used without further purification in the next step.

LC-MS (Method A): Rt=1.14 min

MS (ESIpos): m/z=252 (M+H)⁺

### Intermediate 060

### [3-(2-tert-Butoxyethoxy)phenyl]acetic Acid

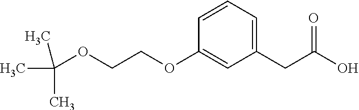 [SMILES: CC(C)(C)OCCOc1cccc(CC(=O)O)c1]

Benzyl (3-hydroxyphenyl)acetate (0.7 g, 2.9 mmol), 2-tert-butoxyethyl 4-methylbenzenesulfonate (1.6 g, 5.8 mmol) and cesium carbonate (1.9 g, 5.8 mmol) were dissolved in dimethylformamide (9 mL) under argon atmosphere and stirred for 72 h at 50° C. After cooling to room temperature, dichloromethane and water were added. The organic phase was separated, dried and concentrated. The crude was purified by column chromatography (silica gel, gradient n-hexane/ethyl acetate) to yield benzyl [3-(2-tert-butoxyethoxy)phenyl]acetate (1.6 g, 2.3 mmol, 50% purity).

Benzyl [3-(2-tert-butoxyethoxy)phenyl]acetate (1.6 g, 2.3 mmol) was converted according to GP 2.1 to [3-(2-tert-butoxyethoxy)phenyl]acetic acid. Purification was done by column chromatography on a Biotage Isolera (silica gel, gradient n-hexane/ethyl acetate) to yield 1.32 g (2.6 mmol, 50% purity). The compound was used in the next step without further purification.

LC-MS (Method A): Rt=1.09 min

MS (ESIneg): m/z=251 (M−H)⁺

### Intermediate 061

### [2-(2-tert-Butoxyethoxy)phenyl]acetic Acid

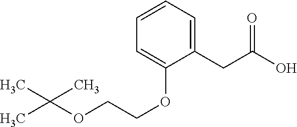 [SMILES: CC(C)(C)OCCOc1ccccc1CC(=O)O]

((2-Hydroxyphenyl)acetic acid (10 g, 66 mmol) was dissolved in dimethylformamide (100 mL) and bicarbonate (8.2 g, 98 mmol) were added. (Bromomethyl)benzene (12.4 g, 72 mmol) in dimethylformamide (5 mL) was added dropwise and stirring was continued for 18 h at room temperature. Water and ethyl acetate were added and the phases were separated. The organic phase was dried and the solvent removed under reduced pressure. The crude was recrystallized from n-hexane/methyl tert-butyl ether to yield benzyl (2-hydroxyphenyl)acetate (12.7 g, 52 mmol, 80% yield).

LC-MS (Method A): Rt=1.14 min

MS (ESIpos): m/z=243 (M+H)⁺

In the next step, benzyl (2-hydroxyphenyl)acetate (1 g, 2.9 mmol, 70% purity) and 2-tert-butoxyethyl 4-methylbenzenesulfonate (1.6 g, 5.8 mmol) were dissolved in dimethylformamide (9 mL). Cesium carbonate (1.9 g, 5.8 mmol) was added and the reaction mixture was stirred for 2 days at 50° C. After cooling to room temperature, water and dichloromethane were added and the phases separated. The organic phase was dried and the solvent was removed under reduced pressure. The crude was purified by column chromatography on a Biotage Isolera system (silica gel, gradient n-hexane/ethyl acetate) to yield benzyl [2-(2-tert-butoxyethoxy)phenyl]acetate (1 g, 1.5 mmol, 50% purity).

Benzyl [2-(2-tert-butoxyethoxy)phenyl]acetate (1 g, 1.5 mmol,) was converted to [2-(2-tert-butoxyethoxy)phenyl]acetic acid by GP2.1 and the crude was used without further purification in the next step (726 mg, 1.4 mmol, 50% purity).

### Intermediate 062

### [3-(2-Methoxyethoxy)phenyl]acetic Acid

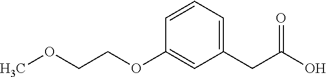

Benzyl (3-hydroxyphenyl)acetate (500 mg, 2.1 mmol) and 2-methoxyethyl 4-methylbenzenesulfonate (1.1 g, 4.1 mmol) were dissolved in dimethylformamide (7 mL). Cesium carbonate (1.4 g, 4.1 mmol) was added and the reaction mixture was stirred for 2 days at 50° C. After cooling to room temperature, water and dichloromethane were added and the phases separated. The organic phase was dried and the solvent was removed under reduced pressure. The crude was purified by column chromatography (silica gel, gradient n-hexane/ethyl acetate) to yield benzyl [3-(2-methoxyethoxy)phenyl]acetate (360 mg, 1.2 mmol, 60% yield, 80% purity).

Benzyl [3-(2-methoxyethoxy)phenyl]acetate (360 mg, 1.2 mmol) was converted to [3-(2-methoxyethoxy)phenyl]acetic acid by GP2.1 and the crude was used without further purification in the next step (117 mg, 0.6 mmol, 47% yield).

¹H-NMR (CDCl₃) δ [ppm]: 3.46 (s, 3H), 3.61 (s, 2H), 3.73-3.78 (m, 2H), 4.08-4.15 (m, 2H), 6.82-6.91 (m, 3H), 7.24 (t, 1H).

### Intermediate 063

### {2-[(2-Methoxyethyl)(methyl)carbamoyl]phenyl}acetic Acid

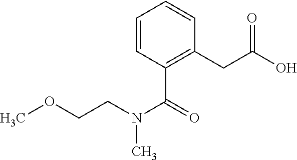 [SMILES: COCCN(C)C(=O)c1ccccc1CC(=O)O]

(2-Bromophenyl)acetic acid (15.0 g, 70 mmol) was dissolved in tetrahydrofurane (45 mL) and trifluoroacetic anhydride (25 mL, 36 g, 174 mmol) was added at 0° C. After 1 h, tert-butanol (103 g, 1.4 mol) was added dropwise and the reaction was stirred at room temperature until TLC showed disappearance of starting material. The reaction was cooled to 0° C. and quenched by addition of saturated aq. bicarbonate solution (100 mL). Ethyl acetate was added and the phases were separated. The organic phase was dried and the solvent was removed under reduced pressure. The crude was used without further purification in the next step.

tert-Butyl (2-bromophenyl)acetate (500 mg, 1.8 mmol), 2-methoxy-N-methylethanamine (493 mg, 5.5 mmol), tri-tert-butylphosphonium tetrafluoroborate (53 mg, 0.184 mmol), palladium(II) acetate (41 mg, 0.184 mmol), carbon monooxide-molybdenum (6:1) (486 mg, 1.8 mmol) and sodium carbonate (586 mg, 5.5 mmol) were dissolved in dioxane (29 mL) under argon atmosphere. Drops of water were added and the reaction was heated for 20 min at 140° C. in the microwave (100 W, 4 bar). After cooling to room temperature, the reaction mixture was filtered and concentrated. The crude was purified by preparative HPLC (Waters XBrigde C18 5μ 100×30 mm, acetonitrile/water+0.2% aqueous ammonia (32%)) to yield {2-[(2-methoxyethyl)(methyl)carbamoyl]phenyl}acetic acid (60 mg, 0.191 mmol, 10% yield).

LC-MS (Method A): Rt=1.12 min

MS (ESIpos): m/z=308 (M+H)⁺

{2-[(2-Methoxyethyl)(methyl)carbamoyl]phenyl}acetic acid (54 mg, 0.175 mmol) was converted according to GP4 to {2-[(2-methoxyethyl)(methyl)carbamoyl]phenyl}acetic acid (45 mg, 0.179 mmol, quant). The crude was co-distilled twice with toluene and used without further purification in the next step.

LC-MS (Method A): Rt=0.59 min

MS (ESIpos): m/z=252 (M+H)⁺

### Intermediate 064

### [3-(Dimethylcarbamoyl)phenyl]acetic Acid

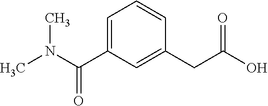

tert-Butyl (3-bromophenyl)acetate (500 mg, 1.8 mmol), N-methylmethanamine (2.8 mL, 250 mg, 5.5 mmol), tri-tert-butylphosphonium tetrafluoroborate (53 mg, 0.184 mmol), trans-Bis(acetato)bis[o-(di-o-tolylphosphino)benzyl]dipalladium(II) (173 mg, 0.184 mmol), carbon monooxide-molybdenum (6:1) (486 mg, 1.8 mmol) and 1,8-diazabicyclo(5.4.0)undec-7-en (842 mg, 5.5 mmol) were dissolved in tetrahydrofurane (14 mL) under argon atmosphere. Drops of water were added and the reaction was heated for 20 min at 125° C. in the microwave (100 W, 7 bar). After cooling to room temperature, the reaction mixture was filtered and concentrated. The crude was purified by preparative HPLC (Waters XBrigde C18 5μ 100×30 mm, acetonitrile/water+0.2% aqueous ammonia (32%)) to yield tert-butyl [3-(dimethylcarbamoyl)phenyl]acetate (39 mg, 0.148 mmol, 8% yield).

LC-MS (Method A): Rt=1.07 min

MS (ESIneg): m/z=262 (M−H)⁺

tert-Butyl [3-(dimethylcarbamoyl)phenyl]acetate (39 mg, 0.148 mmol) was converted according to GP4 to [3-(dimethylcarbamoyl)phenyl]acetic acid (60 mg, 0.289 mmol, quant). The crude was co-distilled twice with toluene and used without further purification in the next step.

### Intermediate 065

### [2-(Dimethylcarbamoyl)phenyl]acetic Acid

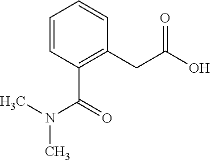

tert-Butyl (3-bromophenyl)acetate (500 mg, 1.8 mmol), N-methylmethanamine (2.8 mL, 250 mg, 5.5 mmol), tri-tert-butylphosphonium tetrafluoroborate (53 mg, 0.184 mmol), trans-Bis(acetato)bis[o-(di-o-tolylphosphino)benzyl]dipalladium(II) (173 mg, 0.184 mmol), carbon monooxide-molybdenum (6:1) (486 mg, 1.8 mmol) and sodium carbonate (586 mg, 5.5 mmol) were dissolved in dioxane (10 mL) under argon atmosphere. Drops of water were added and the reaction was heated for 20 min at 140° C. in the microwave (100 W, 7 bar). After cooling to room temperature, the reaction mixture was filtered and concentrated. The crude was purified by preparative HPLC (Waters XBrigde C18 5μ 100×30 mm, acetonitrile/water+0.2% aqueous ammonia (32%)) to yield tert-butyl [2-(dimethylcarbamoyl)phenyl]acetate (127 mg, 0.48 mmol, 26% yield).

LC-MS (Method A): Rt=1.08 min

MS (ESIpos): m/z=264 (M+H)⁺

tert-Butyl [2-(dimethylcarbamoyl)phenyl]acetate (127 mg, 0.48 mmol) was converted according to GP4 to [2-(dimethylcarbamoyl)phenyl]acetic acid (100 mg, 0.482 mmol, quant). The crude was co-distilled twice with toluene and used without further purification in the next step.

### Intermediate 066

### {3-[(2-Methoxyethyl)carbamoyl]phenyl}acetic Acid

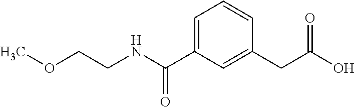 [SMILES: COCCNC(=O)c1cccc(CC(=O)O)c1]

tert-Butyl (3-bromophenyl)acetate (250 mg, 0.9 mmol), 2-methoxyethanamine (0.3 mL, 207 mg, 2.8 mmol), tri-tert-butylphosphonium tetrafluoroborate (28 mg, 0.092 mmol), trans-Bis(acetato)bis[o-(di-o-tolylphosphino)benzyl]dipalladium(II) (86 mg, 0.092 mmol), carbon monooxide-molybdenum (6:1) (243 mg, 0.9 mmol) and 1,8-diazabicyclo(5.4.0)undec-7-en (421 mg, 2.7 mmol) were dissolved in tetrahydrofurane (6 mL) under argon atmosphere. Drops of water were added and the reaction was heated for 20 min at 125° C. in the microwave (100 W, 7 bar). After cooling to room temperature, the reaction mixture was filtered and concentrated. The crude was purified by preparative HPLC (Waters XBrigde C18 5μ 100×30 mm, acetonitrile/water+0.2% aqueous ammonia (32%)) to yield tert-butyl {3-[(2-methoxyethyl)carbamoyl]phenyl}acetate (70 mg, 0.239 mmol, 26% yield).

tert-Butyl {3-[(2-methoxyethyl)carbamoyl]phenyl}acetate (70 mg, 0.239 mmol) was converted according to GP4 to {3-[(2-methoxyethyl)carbamoyl]phenyl}acetic acid (65 mg, 0.274 mmol, quant). The crude was co-distilled twice with toluene and used without further purification in the next step.

### Intermediate 067

### [3-(Methylcarbamoyl)phenyl]acetic Acid

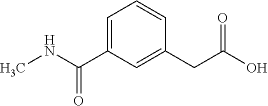

tert-Butyl (3-bromophenyl)acetate (500 mg, 1.8 mmol), methylmethanamine (2.8 mL, 172 mg, 5.5 mmol), tri-tert-butylphosphonium tetrafluoroborate (53 mg, 0.184 mmol), trans-Bis(acetato)bis[o-(di-o-tolylphosphino)benzyl]dipalladium(II) (173 mg, 0.184 mmol) and carbon monooxide-molybdenum (6:1) (486 mg, 1.8 mmol) and 1,8-diazabicyclo(5.4.0)undec-7-en (842 mg, 5.5 mmol) were dissolved in tetrahydrofurane (14 mL) under argon atmosphere. Drops of water were added and the reaction was heated for 20 min at 125° C. in the microwave (100 W, 7 bar). After cooling to room temperature, the reaction mixture was filtered and concentrated. The crude was purified by preparative HPLC (Waters XBrigde C18 5μ 100×30 mm, acetonitrile/water+0.2% aqueous ammonia (32%)) to yield tert-butyl [3-(methylcarbamoyl)phenyl]acetate (30 mg, 0.120 mmol, 7% yield).

LC-MS (Method A): Rt=1.00 min

MS (ESIneg): m/z=248 (M−H)⁺

tert-Butyl [3-(methylcarbamoyl)phenyl]acetate (30 mg, 0.120 mmol) was converted according to GP4 to [3-(methylcarbamoyl)phenyl]acetic acid (60 mg, 0.310 mmol, quant). The crude was co-distilled twice with toluene and used without further purification in the next step.

### Intermediate 068

### N-(2,4-Dimethoxybenzyl)-2-fluoro-3-methyl-5-nitrobenzenesulfonamide

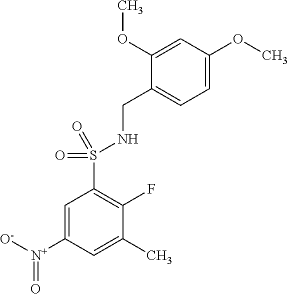 [SMILES: COc1ccc(CNS(=O)(=O)c2cc([N+](=O)[O-])cc(C)c2F)c(OC)c1]

2-Fluoro-3-methyl-5-nitrobenzenesulfonyl chloride (1.00 g, 3.94 mmol) was dissolved in dichloromethane (500 mL) and sodium bicarbonate (662 mg, 7.89 mmol) was added. It was cooled to 0° C. and slowly a solution of 1-(2,4-dimethoxyphenyl)methanamine (659 mg, 3.94 mmol) in dichloromethane (250 mL) was added. The reaction mixture was stirred and allowed to warm to room temperature overnight. Water was added, the phases were separated and the organic phase was dried over sodium sulfate and concentrated in vacuo to yield crude N-(2,4-dimethoxybenzyl)-2-fluoro-3-methyl-5-nitrobenzenesulfonamide (1.51 g, quant.).

LC-MS (Method A): Rt=1.16 min

MS (ESIneg): m/z=383 (M−H)⁺

### Intermediate 069

### 5-Amino-2-(3-chlorophenoxy)-N-(2,4-dimethoxybenzyl)-3-methylbenzenesulfonamide

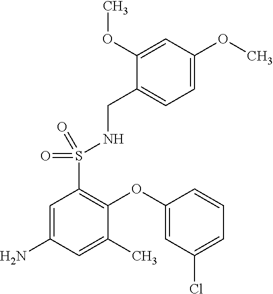 [SMILES: COc1ccc(CNS(=O)(=O)c2cc(N)cc(C)c2Oc2cccc(Cl)c2)c(OC)c1]

Cesium carbonate (1.27 g, 3.90 mmol) was added to a solution of crude N-(2,4-dimethoxybenzyl)-2-fluoro-3-methyl-5-nitrobenzenesulfonamide (1.50 g, 3.90 mmol) in acetonitrile (20 mL). It was cooled to 0° C. and 3-chlorophenol (502 mg, 3.90 mmol) was added. The reaction mixture was stirred and allowed to warm to room temperature overnight. The solvent was removed in vacuo, water and dichloromethane were added, the phases were separated and the organic phase was dried over sodium sulfate and concentrated in vacuo.

The crude product was redissolved in dioxane (30 mL) and tin(II)chloride dihydrate (4.58 g, 20.2 mmol) was added. The reaction mixture was stirred for 2 h at 70° C. After cooling to room temperature the reaction mixture was filtered and concentrated in vacuo to yield crude 5-amino-2-(3-chlorophenoxy)-N-(2,4-dimethoxybenzyl)-3-methylbenzenesulfonamide that was used without further purification in the next step.

LC-MS (Method A): Rt=1.26 min

MS (ESIneg): m/z=461 (M−H)⁺

### Intermediate 070

### 2,2-Dimethyltetrahydro-2H-pyran-4-carboxylic Acid

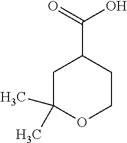

2,2-Dimethyltetrahydro-2H-pyran-4-carbonitrile (900 mg, 6.47 mmol) was refluxed overnight in aqueous 2N KOH solution. It was diluted with water, extracted with ethyl acetate and this organic phase was discarded. The aqueous phase was acidified with 2N HCl solution and extracted twice with ethyl acetate. These organic phases were combined, dried over sodium sulfate and concentrated in vacuo to yield crude 2,2-dimethyltetrahydro-2H-pyran-4-carboxylic acid (889 mg, 5.62 mmol, 87% yield).

¹H-NMR (400 MHz, DMSO-d₆) δ [ppm]: 1.13 (s, 3H), 1.14 (s, 3H), 1.26-1.45 (m, 2H), 1.65-1.73 (m, 2H), 2.59 (tt, 1H), 3.54 (td, 1H), 3.60 (ddd, 1H), 12.20 (s, 1H).

### Intermediate 071

### (2,2-Dimethyltetrahydro-2H-pyran-4-yl)methanol

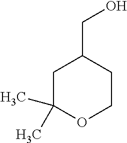

2,2-Dimethyltetrahydro-2H-pyran-4-carboxylic acid (820 mg, 5.18 mmol) was dissolved in tetrahydrofuran (16 mL). At 0° C. BH₃-tetrahydrofuran-complex (668 mg, 7.78 mmol) was added and stirred for another 2 hours at 0° C. It was quenched with saturated ammonium chloride solution, the organic solvent was removed in vacuo, water was added and it was extracted twice with ethyl acetate. The combined organic phases were dried over sodium sulfate and concentrated in vacuo.

As GC-MS showed only 50% conversion the same procedure was repeated leading to crude (2,2-dimethyltetrahydro-2H-pyran-4-yl)methanol (491 mg) which was used in the next step without further purification.

### Intermediate 072

### N-(2,4-dimethoxybenzyl)-2-[(2,2-dimethyltetrahydro-2H-pyran-4-yl)methoxy]-5-nitrobenzenesulfonamide

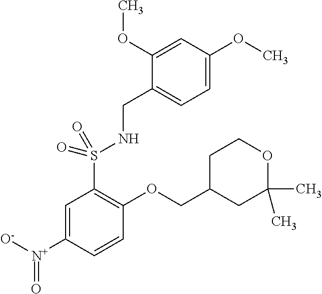 [SMILES: COc1ccc(CNS(=O)(=O)c2cc([N+](=O)[O-])ccc2OCC2CCOC(C)(C)C2)c(OC)c1]

(2,2-Dimethyltetrahydro-2H-pyran-4-yl)methanol (200 mg, 1.39 mmol) and 2-chloro-N-(2,4-dimethoxybenzyl)-5-nitrobenzenesulfonamide (536 mg, 1.39 mmol) were dissolved in acetonitrile (10 mL) and treated with cesium carbonate (452 mg, 1.39 mmol). The reaction mixture was stirred at 110° C. overnight. The solvent was removed in vacuo, water and dichloromethane were added. The organic phase was washed with brine, dried over sodium sulfate and concentrated in vaco. The crude material was purified by column chromatography on a Biotage Isolera system (silica gel, dichloromethane/ethyl acetate) to yield N-(2,4-dimethoxybenzyl)-2-[(2,2-dimethyltetrahydro-2H-pyran-4-yl)methoxy]-5-nitrobenzenesulfonamide (154 mg, 0.311 mmol, 22% yield).

### Intermediate 073

### Methyl 2-(4-{[(2-chlorophenyl)acetyl]amino}-2-[(2,4-dimethoxybenzyl)sulfamoyl]phenoxy)benzoate

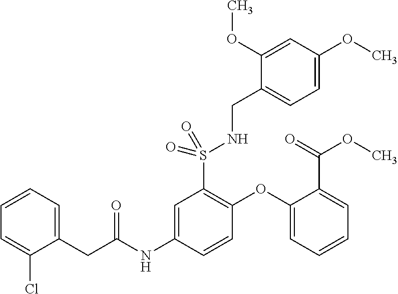 [SMILES: COC(=O)c1ccccc1Oc1ccc(NC(=O)Cc2ccccc2Cl)cc1S(=O)(=O)NCc1ccc(OC)cc1OC]

According to general procedures GP1.1, GP2.1 (with methanol as solvent) and GP3.2 2-chloro-N-(2,4-dimethoxybenzyl)-5-nitrobenzenesulfonamide (1.00 g mg, 2.59 mmol), methyl 2-hydroxybenzoate (393 mg, 2.59 mmol) and (2-chlorophenyl)acetic acid (486 mg, 2.85 mmol) were converted without purification of intermediates to methyl 2-(4-{[(2-chlorophenyl)acetyl]amino}-2-[(2,4-dimethoxybenzyl)sulfamoyl]phenoxy)benzoate and were purified at the end twice by column chromatography on a Biotage Isolera system (silica gel, gradient n-hexane/ethyl acetate and dichloromethane/ethyl acetate) (354 mg, 0.566 mmol, 22% yield over 3 steps).

### Intermediate 074

### Methyl 4-(4-{[(2-chlorophenyl)acetyl]amino}-2-[(2,4-dimethoxybenzyl)sulfamoyl]phenoxy)benzoate

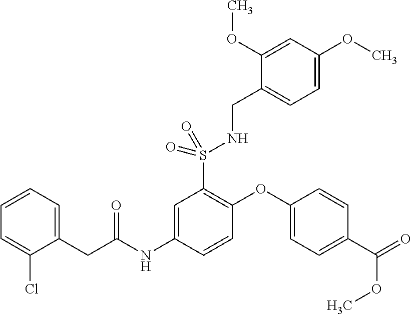 [SMILES: COC(=O)c1ccc(Oc2ccc(NC(=O)Cc3ccccc3Cl)cc2S(=O)(=O)NCc2ccc(OC)cc2OC)cc1]

According to general procedures GP1.1, GP2.1 (with methanol as solvent) and GP3.2 2-chloro-N-(2,4-dimethoxybenzyl)-5-nitrobenzenesulfonamide (1.00 g mg, 2.59 mmol), methyl 4-hydroxybenzoate (393 mg, 2.59 mmol) and (2-chlorophenyl)acetic acid (527 mg, 3.09 mmol) were converted without purification of intermediates to methyl 4-(4-{[(2-chlorophenyl)acetyl]amino}-2-[(2,4-dimethoxybenzyl)sulfamoyl]phenoxy)benzoate and were purified at the end by column chromatography on a Biotage Isolera system (silica gel, gradient n-hexane/ethyl acetate) (441 mg, 0.705 mmol, 27% yield over 3 steps).

### Intermediate 075

### Methyl 3-(4-{[(2-chlorophenyl)acetyl]amino}-2-[(2,4-dimethoxybenzyl)sulfamoyl]phenoxy)benzoate

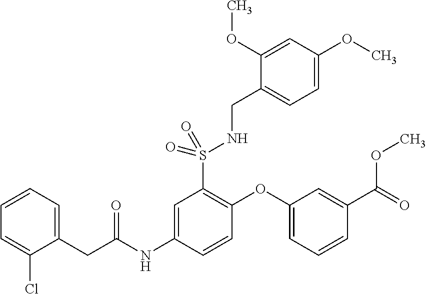 [SMILES: COC(=O)c1cccc(Oc2ccc(NC(=O)Cc3ccccc3Cl)cc2S(=O)(=O)NCc2ccc(OC)cc2OC)c1]

According to general procedures GP1.1, GP2.1 (with methanol as solvent) and GP3.2 2-chloro-N-(2,4-dimethoxybenzyl)-5-nitrobenzenesulfonamide (1.00 g mg, 2.59 mmol), methyl 3-hydroxybenzoate (393 mg, 2.59 mmol) and (2-chlorophenyl)acetic acid (324 mg, 1.90 mmol) were converted without purification of intermediates to methyl 3-(4-{[(2-chlorophenyl)acetyl]amino}-2-[(2,4-dimethoxybenzyl)sulfamoyl]phenoxy)benzoate and were purified at the end by column chromatography on a Biotage Isolera system (silica gel, gradient n-hexane/ethyl acetate) (497 mg, 0.795 mmol, 31% yield over 3 steps).

### Intermediate 076

### Methyl 3-(4-{[(2-chlorophenyl)acetyl]amino}-2-sulfamoylphenoxy)benzoate

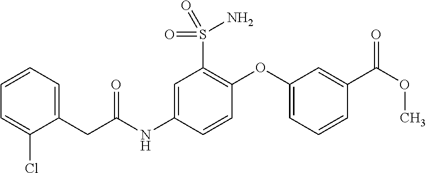 [SMILES: COC(=O)c1cccc(Oc2ccc(NC(=O)Cc3ccccc3Cl)cc2S(N)(=O)=O)c1]

According to GP4 methyl 3-(4-{[(2-chlorophenyl)acetyl]amino}-2-[(2,4-dimethoxybenzyl)sulfamoyl]phenoxy)benzoate (281 mg, 0.45 mmol) was converted to methyl 3-(4-{[(2-chlorophenyl)acetyl]amino}-2-sulfamoylphenoxy)benzoate and was used without further purification in the next step (226 mg).

LC-MS (Method A): Rt=1.11 min

MS (ESIpos): m/z=475 (M+H)⁺

### Intermediate 077

### N-(2,4-Dimethoxybenzyl)-5-nitro-2-(tetrahydro-2H-pyran-4-ylmethoxy)benzenesulfonamide

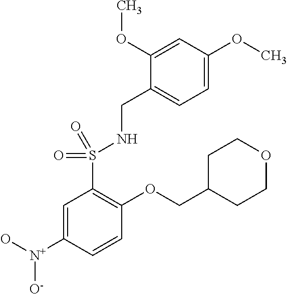 [SMILES: COc1ccc(CNS(=O)(=O)c2cc([NH+]([O-])O)ccc2OCC2CCOCC2)c(OC)c1]

2-Chloro-N-(2,4-dimethoxybenzyl)-5-nitrobenzenesulfonamide (2.00 g, 5.17 mmol) was dissolved in dimethylformamide (10 mL), treated with tetrahydro-2H-pyran-4-ylmethanol (901 mg, 7.76 mmol) and sodium hydride (1.58 g, 36.2 mmoL) and was stirred for 2 hours at room temperature. It was quenched under ice cooling with water/ethyl acetate. The phases were separated, the aqueous phase was three times reextracted with ethyl acetate and all organic phases were combined, dried and concentrated in vacuo. It was then stirred with ethyl acetate/methyl tert.-butyl ether (1/2) until a white solid precipitated. Filtration led to N-(2,4-dimethoxybenzyl)-5-nitro-2-(tetrahydro-2H-pyran-4-ylmethoxy)benzenesulfonamide (2.20 g, 4.75 mmol, 91% yield, 95% purity)

LC-MS (Method A): Rt=1.16 min

MS (ESIneg): m/z=465 (M−H)⁺

¹H-NMR (400 MHz, DMSO-d₆) δ [ppm]: 1.23-1.36 (m, 2H), 1.70-1.77 (m, 2H), 2.09-2.23 (m, 1H), 3.29-3.39 (m, 2H), 3.59 (s, 3H), 3.65 (s, 3H), 3.89 (dd, 2H), 3.99 (d, 2H), 4.08 (s, 2H), 6.21 (d, 1H), 6.30 (dd, 1H), 7.01 (d, 1H), 7.25 (d, 1H), 7.42 (s, 1H), 8.23 (d, 1H), 8.31 (dd, 1H).

### Intermediate 078

### 5-Amino-N-(2,4-dimethoxybenzyl)-2-(tetrahydro-2H-pyran-4-ylmethoxy)benzenesulfonamide

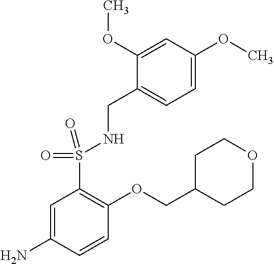 [SMILES: COc1ccc(CNS(=O)(=O)c2cc(N)ccc2OCC2CCOCC2)c(OC)c1]

N-(2,4-dimethoxybenzyl)-5-nitro-2-(tetrahydro-2H-pyran-4-ylmethoxy)benzenesulfonamide (2.20 g, 4.75 mmol) was dissolved in methanol, treated with Pd/C (10% loading) and stirred under a hydrogen atmosphere for 3 days at room temperature. After filtration, the filtrate was concentrated in vacuo to give 5-amino-N-(2,4-dimethoxybenzyl)-2-(tetrahydro-2H-pyran-4-ylmethoxy)benzenesulfonamide (1.55 g, 3.54 mmol, 75% yield), which was used without further purification in the following steps.

LC-MS (Method A): Rt=0.92 min

MS (ESIpos): m/z=437 (M+H)⁺

¹H-NMR (400 MHz, DMSO-d₆) δ [ppm]: 1.25 (ddd, 2H), 1.63-1.70 (m, 2H), 1.90-2.03 (m, 1H), 3.25-3.33 (m, 2H), 3.68-3.74 (m, 8H), 3.85 (dd, 2H), 3.94 (d, 2H), 5.10 (s, 2H), 6.40 (dd, 1H), 6.46 (d, 1H), 6.54 (t, 1H), 6.72 (dd, 1H), 6.84 (d, 1H), 7.01 (d, 1H), 7.05 (d, 1H).

### Intermediate 079

### 5-Amino-2-(3-chlorophenoxy)-N-(2,4-dimethoxybenzyl)benzenesulfonamide

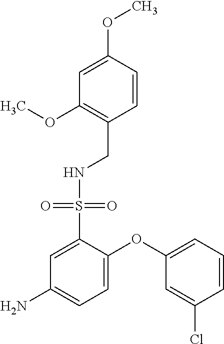 [SMILES: COc1ccc(CNS(=O)(=O)c2cc(N)ccc2Oc2cccc(Cl)c2)c(OC)c1]

2-Chloro-N-(2,4-dimethoxybenzyl)-5-nitrobenzenesulfonamide (62.4 g, 161 mmol) was dissolved in acetonitrile (620 mL), cesium carbonate (52.6 g, 161 mmol) and 3-chlorophenol (20.7 g, 161 mmol) were added. The reaction mixture was stirred overnight at 110° C. (bath temperature). Dichloromethane (620 mL) was added and it was stirred for 30 min. The precipitate was discarded. The filtrate was concentrated in vacuo, suspended in dichloromethane (500 mL) and purified over a silica bed (dichloromethane as eluent). After concentration in vacuo the material (53 g) was suspended in a mixture of acetonitrile (530 mL) and dichloromethane (530 mL) and stirred for 30 min. The precipitate was collected and dried to provide 2-(3-chlorophenoxy)-N-(2,4-dimethoxybenzyl)-5-nitrobenzenesulfonamide (30.0 g)

LC-MS (Method B): Rt=1.39 min

MS (ESIneg): m/z=477 (M−H)−

The material from the previous step was suspended in toluene (390 mL). Water (390 mL), phosphoric acid (40 μL to reach pH 3) and platinum/vanadium (1%/2%) on charcoal (10 g) were added. The reaction mixture was stirred for 4 h at 100° C. under hydrogen atmosphere (6.25 bar) in an autoclave. The catalyst was filtered off and the filtrate was extracted with ethylacetate/water. The aqueous phase was reextracted three times with ethyl acetate The organic phases were combined, washed with brine, dried over sodium sulfate and were concentrated in vacuo. Purification on a Biotage Isolera system (hexane/ethyl acetate 1/1) provided 5-amino-2-(3-chlorophenoxy)-N-(2,4-dimethoxybenzyl)benzenesulfonamide (28.8 g, 64.2 mmol, 40% yield over 2 steps).

LC-MS (Method B): Rt=1.26 min

MS (ESIpos): m/z=449 (M+H)⁺

¹H-NMR (400 MHz, DMSO-d₆) δ [ppm]: 3.71 (d, 3H), 3.72 (d, 3H), 3.98 (d, 2H), 5.46 (s, 2H), 6.40-6.47 (m, 2H), 6.75 (dd, 1H), 6.79-6.85 (m, 2H), 6.90 (t, 1H), 7.04-7.10 (m, 2H), 7.13 (d, 1H), 7.31 (t, 1H), 7.44 (t, 1H).

### Intermediate 080

### 5-Amino-2-(3-chlorophenoxy)-N-(2,4-dimethoxybenzyl)-3-fluorobenzenesulfonamide

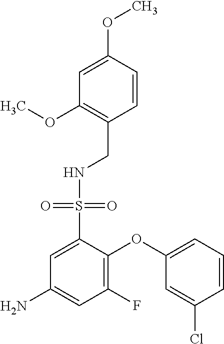 [SMILES: COc1ccc(CNS(=O)(=O)c2cc(N)cc(F)c2Oc2cccc(Cl)c2)c(OC)c1]

N-(2,4-Dimethoxybenzyl)-2,3-difluoro-5-nitrobenzenesulfonamide (1.51 g, 3.88 mmol) was dissolved in acetonitrile (50 mL). Under ice cooling, cesium carbonate (1.26 g, 3.88 mmol) and 3-chlorophenol (499 mg, 3.88 mmol) in acetonitrile (20 mL) were slowly added. The reaction mixture was stirred and allowed to warm to room temperature overnight. After concentration in vacuo it was extracted with ethyl acetate/water, the organic phase was dried over sodium sulfate and concentrated again in vacuo to give crude 2-(3-chlorophenoxy)-N-(2,4-dimethoxybenzyl)-3-fluoro-5-nitrobenzenesulfonamide.

The material from the previous step was dissolved in dioxane (15 mL) and treated with tin(II) chloride dihydrate for 2 h at 70° C. After cooling to room temperature the reaction mixture was filtered and the filtrate was concentrated in vacuo to give crude 5-amino-2-(3-chlorophenoxy)-N-(2,4-dimethoxybenzyl)-3-fluorobenzenesulfonamide that was used in the next step without further purification.

### Intermediate 081

### 5-Amino-2-(3-chlorophenoxy)pyridine-3-sulfonamide

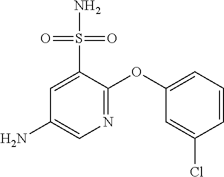 [SMILES: Nc1cnc(Oc2cccc(Cl)c2)c(S(N)(=O)=O)c1]

3-Chlorophenol (1.89 g, 14.7 mmol) was stirred for 30 min in aqueous 10% sodium hydroxide solution (5.36 mL, 14.7 mmol), followed by concentration in vacuo to generate the corresponding alcoholate.

A suspension of this alcoholate, further 3-chlorophenol (946 mg, 7.37 mmol), 5-bromo-2-chloropyridine-3-sulfonamide (2.00 g, 7.37 mmol), cesium carbonate (4.8 g, 14.7 mmol) and potassium carbonate (4.07 g, 29.4 mmol) in acetonitrile (30 mL) was irradiated in the microwave (150° C., 1 h). Afterwards the reaction mixture was concentrated in vacuo, extracted with ethyl acetate/water and the organic phase was dried over sodium sulfate, concentrated in vacuo and purified with a Biotage Isolera system providing 5-bromo-2-(3-chlorophenoxy)pyridine-3-sulfonamide (2.67 g).

LC-MS (Method A): Rt=1.11 min

MS (ESIpos): m/z=363/365 (M+H)⁺

The material from the previous step was added to a solution of 1,1-dimethoxy-N,N-dimethylmethanamine (1.70 g, 14.3 mmol) in DMF (30 mL) and was stirred for 1 h at room temperature. The reaction mixture was concentrated in vacuo and extracted with ethyl acetate/water. The organic phase was dried over sodium sulfate and concentrated in vacuo.

LC-MS (Method A): Rt=1.18 min

MS (ESIpos): m/z=418/420 (M+H)⁺

¹H-NMR (400 MHz, DMSO-d₆) δ [ppm]: 2.91 (s, 3H), 3.04 (s, 3H), 7.07 (ddd, 1H), 7.23 (t, 1H), 7.34 (ddd, 1H), 7.47 (t, 1H), 8.25 (s, 1H), 8.37 (d, 1H), 8.51 (d, 1H).

The material from the previous step was dissolved in dioxane (10 mL) and flushed with argon. Xantphos (4,5-Bis(diphenylphosphino)-9,9-dimethyxanthene) (138 mg, 0.239 mmol) and palladium(II) acetate (26.8 mg, 0.118 mmol) were added, followed by a second argon flushing. Then, cesium carbonate (2.33 g, 7.16 mmol) and 1,1-diphenylmethanimine (649 mg, 3.58 mmol) were added and the reaction mixture was stirred overnight at 95° C. After concentration in vacuo it was extracted with ethyl acetate/water and the organic phase was dried over sodium sulfate and concentrated again in vacuo to give crude 2-(3-chlorophenoxy)-N-[(dimethylamino)methylene]-5-[(diphenylmethylene)amino]pyridine-3-sulfonamide (1.20 g) that was used without further purification in the next step.

LC-MS (Method A): Rt=1.39 min

MS (ESIpos): m/z=519 (M+H)⁺

The material from the previous step was dissolved in ethanol (150 mL), 4N HCl in dioxane (5.78 mL) was added and it was stirred at room temperature until complete conversion. Concentration in vacuo was followed by purification on a Biotage Isolera system to yield 5-amino-2-(3-chlorophenoxy)pyridine-3-sulfonamide (450 mg, 1.50 mmol, 20% yield over 4 steps, 85% purity)

LC-MS (Method A): Rt=0.82 min

MS (ESIpos): m/z=300 (M+H)⁺

¹H-NMR (400 MHz, DMSO-d₆) δ [ppm]: 5.59 (s, 2H), 7.03 (ddd, 1H), 7.12 (t, 1H), 7.20 (ddd, 1H), 7.39 (t, 1H), 7.44 (s, 2H), 7.54 (d, 1H), 7.67 (d, 1H).

### Synthesis of Examples

### Example 001

### N-[4-(3-Chloro-5-cyanophenoxy)-3-sulfamoylphenyl]-2-(2-chlorophenyl)acetamide

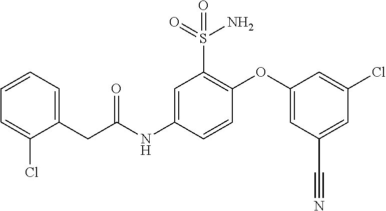 [SMILES: N#Cc1cc(Cl)cc(Oc2ccc(NC(=O)Cc3ccccc3Cl)cc2S(N)(=O)=O)c1]

Example 1 was synthesized according to general procedures GP1.1, GP2.2, GP3 and GP4 without purification of intermediates as following:

2-Chloro-N-(2,4-dimethoxybenzyl)-5-nitrobenzenesulfonamide (500 mg, 1.29 mmol) was dissolved in acetonitrile (10 mL) and cesium carbonate (421 mg, 1.29 mmol) and 3-chloro-5-hydroxybenzonitrile (199 mg, 1.29 mmol) were added. Stirring was continued overnight. Afterwards, all volatile components were removed in vacuo, followed by addition of water and dichloromethane. The phases were separated, the organic phase was removed and dried over sodium sulfate and concentrated in vacuo to obtain crude 2-(3-chloro-5-cyanophenoxy)-N-(2,4-dimethoxybenzyl)-5-nitrobenzenesulfonamide.

LC-MS (Method A): Rt=1.33 min

MS (ESIneg): m/z=502 (M−H)⁺

To a solution of crude 2-(3-chloro-5-cyanophenoxy)-N-(2,4-dimethoxybenzyl)-5-nitrobenzenesulfonamide from the previous step in dioxane (6 mL) was added tin(II) chloride hydrate (1.46 g, 6.46 mmol). The reaction mixture was stirred for 2 h at 70° C. Then the reaction mixture was cooled to room temperature and the resulting precipitate was removed by filtration. The filtrate was concentrated in vacuo to obtain crude 5-amino-2-(3-chloro-5-cyanophenoxy)-N-(2,4-dimethoxybenzyl)benzenesulfonamide.

LC-MS (Method A): Rt=1.17 min

MS (ESIpos): m/z=474 (M+H)⁺

To a solution of crude 5-amino-2-(3-chloro-5-cyanophenoxy)-N-(2,4-dimethoxybenzyl)benzenesulfonamide from the previous step in tetrahydrofuran (10 mL) was added (2-chlorophenyl)acetic acid (330 mg, 1.94 mmol), N,N-diisopropylethylamine (1.67 g, 12.9 mmol) and HATU (736 mg, 1.94 mmol). The reaction mixture was stirred overnight at room temperature. Then it was concentrated in vacuo, followed by extraction from ethyl acetate/water. The organic phase was washed with water, dried over sodium sulfate and concentrated in vacuo to obtain crude N-{4-(3-chloro-5-cyanophenoxy)-3-[(2,4-dimethoxybenzyl)sulfamoyl]phenyl}-2-(2-chlorophenyl)acetamide.

LC-MS (Method A): Rt=1.34 min

MS (ESIneg): m/z=624 (M−H)⁺

To a solution of crude N-{4-(3-chloro-5-cyanophenoxy)-3-[(2,4-dimethoxybenzyl)sulfamoyl]phenyl}-2-(2-chlorophenyl)acetamide from the previous step in dichloromethane (10 mL) was added trifluoroacetic acid (7.36 g, 64.6 mmol) and the reaction mixture was stirred for 1 h at room temperature. All volatile components were removed in vacuo and the resulting residue was purified by preparative HPLC (Waters XBrigde C18 5μ 100×30 mm, acetonitrile/water+0.1% formic acid) to obtain N-[4-(3-chloro-5-cyanophenoxy)-3-sulfamoylphenyl]-2-(2-chlorophenyl)acetamide (97 mg, 95% purity, 0.204 mmol, 16% yield over 4 steps).

LC-MS (Method A): Rt=1.16 min

MS (ESIpos): m/z=476 (M+H)⁺

¹H-NMR (400 MHz, DMSO-d₆) δ [ppm]: 3.78 (s, 2H), 6.88 (d, 1H), 7.24-7.34 (m, 2H), 7.34-7.47 (m, 4H), 7.56-7.66 (m, 2H), 8.06 (d, 1H), 10.23 (s, 1H), 10.75 (s, 2H).

### Example 002

### 2-(2-Chlorophenyl)-N-{4-[3-(dimethylamino)phenoxy]-3-sulfamoylphenyl}acetamide

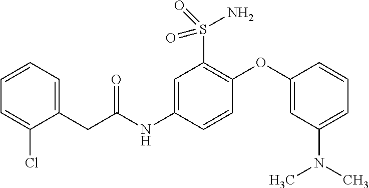 [SMILES: CN(C)c1cccc(Oc2ccc(NC(=O)Cc3ccccc3Cl)cc2S(N)(=O)=O)c1]

According to general procedures GP1.1, GP2.2, GP3.2 and GP4, 2-chloro-N-(2,4-dimethoxybenzyl)-5-nitrobenzenesulfonamide (1.06 g, 2.73 mmol), 3-(dimethylamino)phenol (374 mg, 2.73 mmol) and (2-chlorophenyl)acetic acid (545 mg, 3.19 mmol) were converted without purification of intermediates to 2-(2-chlorophenyl)-N-{4-[3-(dimethylamino)phenoxy]-3-sulfamoylphenyl}acetamide and were purified at the end by column chromatography on a Biotage Isolera system (silica gel, gradient n-hexane/ethyl acetate), followed by preparative HPLC (Waters XBrigde C18 5μ 100×30 mm, acetonitrile/water+0.1% formic acid) (30 mg, 0.0652 mmol, 2% yield over 4 steps, 98% purity).

LC-MS (Method A): Rt=1.15 min

MS (ESIpos): m/z=460 (M+H)⁺

¹H-NMR (400 MHz, DICHLOROMETHANE-d₂) δ [ppm]: 2.96 (s, 6H), 3.88 (s, 2H), 5.20 (s, 2H), 6.37 (dd, 1H), 6.47 (t, 1H), 6.59 (dd, 1H), 6.95 (d, 1H), 7.24 (t, 1H), 7.31-7.38 (m, 2H), 7.40-7.52 (m, 3H), 7.78 (dd, 1H), 7.88 (d, 1H).

### Example 003

### 2-(2-Chlorophenyl)-N-{4-[(2-chloropyridin-4-yl)oxy]-3-sulfamoylphenyl}acetamide

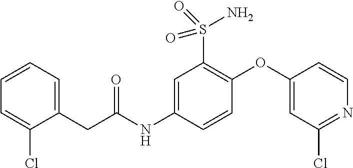 [SMILES: NS(=O)(=O)c1cc(NC(=O)Cc2ccccc2Cl)ccc1Oc1ccnc(Cl)c1]

According to general procedures GP1.1, GP2.2, GP3.2 and GP4, 2-chloro-N-(2,4-dimethoxybenzyl)-5-nitrobenzenesulfonamide (1.03 mg, 2.66 mmol), 2-chloropyridin-4-ol (344 mg, 2.63 mmol) and (2-chlorophenyl)acetic acid (681 mg, 3.99 mmol) were converted without purification of intermediates to 2-(2-chlorophenyl)-N-{4-[(2-chloropyridin-4-yl)oxy]-3-sulfamoylphenyl}acetamide and were purified at the end by column chromatography on a Biotage Isolera system (silica gel, gradient n-hexane/ethyl acetate/methanol), followed by preparative HPLC (Chromatorex C-18 10 μm, 125×30 mm, acetonitrile/water+0.1% formic acid) (45 mg, 0.0995 mmol, 4% yield over 4 steps, 98% purity).

LC-MS (Method A): Rt=1.06 min

MS (ESIpos): m/z=452 (M+H)⁺

¹H-NMR (400 MHz, DMSO-d₆) δ [ppm]: 3.82 (s, 2H), 6.91 (d, 1H), 7.01 (d, 1H), 7.05 (s, 1H), 7.27-7.37 (m, 2H), 7.40-7.48 (m, 2H), 7.64 (dd, 1H), 8.13 (d, 1H), 8.18 (d, 1H), 10.29 (s, 1H), 10.71-11.27 (m, 2H).

### Example 004

### 2-(2-Chlorophenyl)-N-[4-(3-isopropylphenoxy)-3-sulfamoylphenyl]acetamide

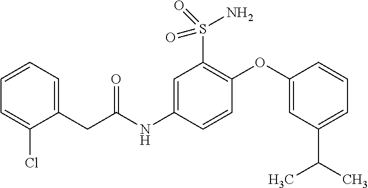 [SMILES: CC(C)c1cccc(Oc2ccc(NC(=O)Cc3ccccc3Cl)cc2S(N)(=O)=O)c1]

According to general procedures GP1.1, GP2.2, GP3.2 and GP4, 2-chloro-N-(2,4-dimethoxybenzyl)-5-nitrobenzenesulfonamide (1025 mg, 2.65 mmol), 3-isopropylphenol (361 mg, 2.65 mmol) and (2-chlorophenyl)acetic acid (499 mg, 2.93 mmol) were converted without purification of intermediates to 2-(2-chlorophenyl)-N-[4-(3-isopropylphenoxy)-3-sulfamoylphenyl]acetamide and were purified at the end by column chromatography on a Biotage Isolera system (silica gel, gradient n-hexane/ethyl acetate) (251 mg, 0.547 mmol, 21% yield over 4 steps, 95% purity).

LC-MS (Method A): Rt=1.33 min

MS (ESIpos): m/z=459 (M+H)⁺

¹H-NMR (400 MHz, DICHLOROMETHANE-d₂) δ [ppm]: 1.26 (d, 6H), 2.93 (sep, 1H), 3.86 (s, 2H), 5.22 (s, 2H), 6.86-6.90 (m, 2H), 7.00 (t, 1H), 7.12 (d, 1H), 7.29-7.36 (m, 3H), 7.39-7.49 (m, 2H), 7.52 (s, 1H), 7.78 (dd, 1H), 7.88 (d, 1H).

### Example 005

### 2-(2-Chlorophenyl)-N-{3-sulfamoyl-4-[3-(trifluoromethyl)phenoxy]phenyl}acetamide

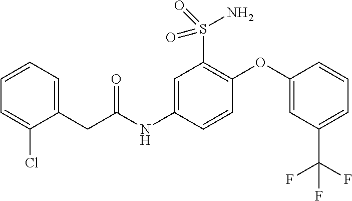 [SMILES: NS(=O)(=O)c1cc(NC(=O)Cc2ccccc2Cl)ccc1Oc1cccc(C(F)(F)F)c1]

According to general procedures GP1.1, GP2.2, GP3.2 and GP4, 2-chloro-N-(2,4-dimethoxybenzyl)-5-nitrobenzenesulfonamide (998 mg, 2.58 mmol), 3-(trifluoromethyl)phenol (418 mg, 2.58 mmol) and (2-chlorophenyl)acetic acid (509 mg, 2.98 mmol) were converted without purification of intermediates to 2-(2-chlorophenyl)-N-{3-sulfamoyl-4-[3-(trifluoromethyl)phenoxy]phenyl}acetamide and were purified at the end by column chromatography on a Biotage Isolera (silica gel, gradient n-hexane/ethyl acetate) (405 mg, 0.835 mmol, 32% yield over 4 steps, 95% purity).

LC-MS (Method A): Rt=1.26 min

MS (ESIpos): m/z=485 (M+H)⁺

¹H-NMR (400 MHz, DICHLOROMETHANE-d₂) δ [ppm]: 3.87 (s, 2H), 5.26 (s, 2H), 6.93 (d, 1H), 7.24-7.28 (m, 1H), 7.29-7.35 (m, 2H), 7.37 (s, 1H), 7.39-7.57 (m, 4H), 7.68 (s, 1H), 7.84 (dd, 1H), 7.95 (d, 1H).

### Example 006

### 2-(2-Chlorophenyl)-N-{3-sulfamoyl-4-[3-(trifluoromethoxy)phenoxy]phenyl}acetamide

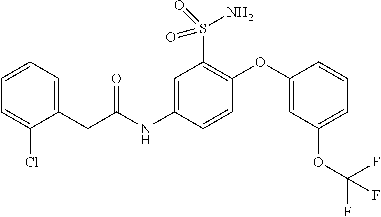 [SMILES: NS(=O)(=O)c1cc(NC(=O)Cc2ccccc2Cl)ccc1Oc1cccc(OC(F)(F)F)c1]

According to general procedures GP1.1, GP2.2, GP3.2 and GP4, 2-chloro-N-(2,4-dimethoxybenzyl)-5-nitrobenzenesulfonamide (1.03 g, 2.66 mmol), 3-(trifluoromethoxy)phenol (473 mg, 2.66 mmol) and (2-chlorophenyl)acetic acid (516 mg, 3.02 mmol) were converted without purification of intermediates to 2-(2-chlorophenyl)-N-{3-sulfamoyl-4-[3-(trifluoromethoxy)phenoxy]phenyl}acetamide and were purified at the end by column chromatography on a Biotage Isolera system (silica gel, gradient n-hexane/ethyl acetate) (227 mg, 0.453 mmol, 17% yield over 4 steps, 97% purity).

LC-MS (Method A): Rt=1.29 min

MS (ESIpos): m/z=501 (M+H)⁺

¹H-NMR (400 MHz, DICHLOROMETHANE-d₂) δ [ppm]: 3.88 (s, 2H), 5.18 (s, 2H), 6.97 (d, 1H), 6.99-7.01 (m, 1H), 7.03 (ddd, 1H), 7.07-7.12 (m, 1H), 7.30-7.37 (m, 2H), 7.40-7.52 (m, 4H), 7.85 (dd, 1H), 7.93 (d, 1H).

### Example 007

### N-[4-(3-Acetylphenoxy)-3-sulfamoylphenyl]-2-(2-chlorophenyl)acetamide

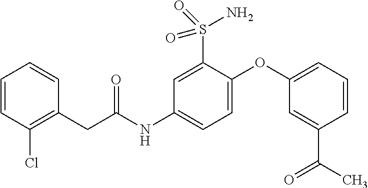 [SMILES: CC(=O)c1cccc(Oc2ccc(NC(=O)Cc3ccccc3Cl)cc2S(N)(=O)=O)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), 1-(3-hydroxyphenyl)ethanone (176 mg, 1.29 mmol) and (2-chlorophenyl)acetic acid (242 mg, 1.42 mmol) were converted without purification of intermediates to N-[4-(3-acetylphenoxy)-3-sulfamoylphenyl]-2-(2-chlorophenyl)acetamide and were purified at the end by preparative HPLC (Waters XBrigde C18 5μ 100×30 mm, acetonitrile/water+0.1% formic acid) (15 mg, 0.0327 mmol, 3% yield over 4 steps, 98% purity).

LC-MS (Method A): Rt=1.08 min

MS (ESIpos): m/z=459 (M+H)⁺

¹H-NMR (400 MHz, DMSO-d₆) δ [ppm]: 2.58 (s, 3H), 3.86 (s, 2H), 7.04 (d, 1H), 7.27-7.36 (m, 3H), 7.40 (s, 2H), 7.42-7.48 (m, 2H), 7.52-7.58 (m, 2H), 7.74-7.78 (m, 1H), 7.80 (dd, 1H), 8.23 (d, 1H), 10.53 (s, 1H).

### Example 008

### N-[4-(1,3-Benzodioxol-5-yloxy)-3-sulfamoylphenyl]-2-(2-chlorophenyl)acetamide

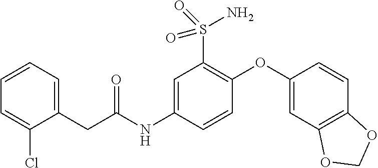 [SMILES: NS(=O)(=O)c1cc(NC(=O)Cc2ccccc2Cl)ccc1Oc1ccc2c(c1)OCO2]

According to general procedures GP1.1, GP2.2, GP3.2 and GP4, 2-chloro-N-(2,4-dimethoxybenzyl)-5-nitrobenzenesulfonamide (1.03 g, 2.66 mmol), 1,3-benzodioxol-5-ol (367 mg, 2.66 mmol) and (2-chlorophenyl)acetic acid (577 mg, 3.38 mmol) were converted without purification of intermediates to N-[4-(1,3-benzodioxol-5-yloxy)-3-sulfamoylphenyl]-2-(2-chlorophenyl)acetamide and were purified at the end by column chromatography on a Biotage Isolera system (silica gel, gradient n-hexane/ethyl acetate) followed by preparative HPLC (Waters XBrigde C18 5μ 100×30 mm, acetonitrile/water+0.1% formic acid) (65 mg, 0.141 mmol, 5% yield over 4 steps, 98% purity).

LC-MS (Method A): Rt=1.10 min

MS (ESIpos): m/z=461 (M+H)⁺

¹H-NMR (400 MHz, DICHLOROMETHANE-d₂) δ [ppm]: 3.88 (s, 2H), 5.21 (s, 2H), 6.04 (s, 2H), 6.60 (dd, 1H), 6.66 (d, 1H), 6.83 (d, 1H), 6.90 (d, 1H), 7.31-7.39 (m, 2H), 7.39-7.51 (m, 3H), 7.79 (dd, 1H), 7.87 (d, 1H).

### Example 009

### N-[4-(3-Acetamidophenoxy)-3-sulfamoylphenyl]-2-(2-chlorophenyl)acetamide

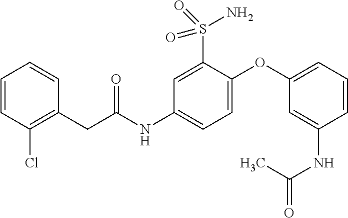 [SMILES: CC(=O)Nc1cccc(Oc2ccc(NC(=O)Cc3ccccc3Cl)cc2S(N)(=O)=O)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), N-(3-hydroxyphenyl)acetamide (195 mg, 1.29 mmol) and (2-chlorophenyl)acetic acid (242 mg, 1.42 mmol) were converted without purification of intermediates to N-[4-(3-acetamidophenoxy)-3-sulfamoylphenyl]-2-(2-chlorophenyl)acetamide and were purified at the end by preparative HPLC (Waters XBrigde C18 5μ 100×30 mm, acetonitrile/water+0.1% formic acid) (15 mg, 0.0317 mmol, 2% yield over 4 steps, 94% purity).

LC-MS (Method A): Rt=0.99 min

MS (ESIpos): m/z=474 (M+H)⁺

¹H-NMR (400 MHz, DMSO-d₆) δ [ppm]: 2.02 (s, 3H), 3.85 (s, 2H), 6.71-6.77 (m, 1H), 6.96 (d, 1H), 7.26-7.40 (m, 7H), 7.41-7.48 (m, 2H), 7.75 (dd, 1H), 8.19 (d, 1H), 10.00 (s, 1H), 10.48 (s, 1H).

### Example 010

### 2-(2-Chlorophenyl)-N-[4-(2-fluorophenoxy)-3-sulfamoylphenyl]acetamide

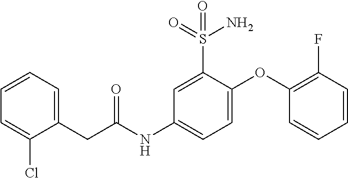 [SMILES: NS(=O)(=O)c1cc(NC(=O)Cc2ccccc2Cl)ccc1Oc1ccccc1F]

According to general procedures GP1.1, GP2.2, GP3.2 and GP4, 2-chloro-N-(2,4-dimethoxybenzyl)-5-nitrobenzenesulfonamide (500 mg, 1.29 mmol), 2-fluorophenol (145 mg, 1.29 mmol) and (2-chlorophenyl)acetic acid (239 mg, 1.40 mmol) were converted without purification of intermediates to 2-(2-chlorophenyl)-N-[4-(2-fluorophenoxy)-3-sulfamoylphenyl]acetamide and were purified at the end by preparative HPLC (Waters XBrigde C18 5μ 100×30 mm, acetonitrile/water+0.1% formic acid) (65 mg, 0.0149 mmol, 12% yield over 4 steps, 98% purity).

LC-MS (Method A): Rt=1.12 min

MS (ESIpos): m/z=435 (M+H)⁺

¹H-NMR (400 MHz, DMSO-d₆) δ [ppm]: 3.82 (s, 2H), 6.80 (d, 1H), 7.12-7.34 (m, 5H), 7.35-7.47 (m, 5H), 7.71 (dd, 1H), 8.18 (d, 1H), 10.43 (s, 1H).

### Example 011

### 2-(2-Chlorophenyl)-N-[4-(3-fluorophenoxy)-3-sulfamoylphenyl]acetamide

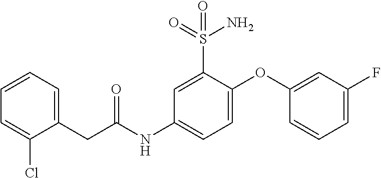 [SMILES: NS(=O)(=O)c1cc(NC(=O)Cc2ccccc2Cl)ccc1Oc1cccc(F)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), 3-fluorophenol (145 mg, 1.29 mmol) and (2-chlorophenyl)acetic acid (239 mg, 1.40 mmol) were converted without purification of intermediates to 2-(2-chlorophenyl)-N-[4-(3-fluorophenoxy)-3-sulfamoylphenyl]acetamide and were purified at the end by preparative HPLC (Waters XBrigde C18 5μ 100×30 mm, acetonitrile/water+0.1% formic acid) (66 mg, 0.0152 mmol, 12% yield over 4 steps, 98% purity).

LC-MS (Method A): Rt=1.14 min

MS (ESIpos): m/z=435 (M+H)⁺

¹H-NMR (400 MHz, DMSO-d₆) δ [ppm]: 3.84 (s, 2H), 6.81-6.91 (m, 2H), 6.96 (tdd, 1H), 7.07 (d, 1H), 7.26-7.47 (m, 7H), 7.78 (dd, 1H), 8.20 (d, 1H), 10.50 (s, 1H).

### Example 012

### 2-(2-chlorophenyl)-N-[4-(4-fluorophenoxy)-3-sulfamoylphenyl]acetamide

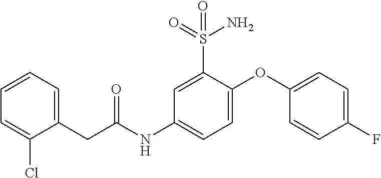 [SMILES: NS(=O)(=O)c1cc(NC(=O)Cc2ccccc2Cl)ccc1Oc1ccc(F)cc1]

According to general procedures GP1.1, GP2.2, GP3.2 and GP4, 2-chloro-N-(2,4-dimethoxybenzyl)-5-nitrobenzenesulfonamide (500 mg, 1.29 mmol), 4-fluorophenol (145 mg, 1.29 mmol) and (2-chlorophenyl)acetic acid (239 mg, 1.40 mmol) were converted without purification of intermediates to 2-(2-chlorophenyl)-N-[4-(4-fluorophenoxy)-3-sulfamoylphenyl]acetamide and were purified at the end by preparative HPLC (Waters XBrigde C18 5μ 100×30 mm, acetonitrile/water+0.1% formic acid) (44 mg, 0.101 mmol, 8% yield over 4 steps, 98% purity).

LC-MS (Method A): Rt=1.14 min

MS (ESIpos): m/z=435 (M+H)⁺

¹H-NMR (400 MHz, DMSO-d₆) δ [ppm]: 3.82 (s, 2H), 6.92 (d, 1H), 7.04-7.12 (m, 2H), 7.18-7.26 (m, 2H), 7.27-7.37 (m, 4H), 7.38-7.46 (m, 2H), 7.74 (dd, 1H), 8.18 (d, 1H), 10.45 (s, 1H).

### Example 013

### 2-(2-Chlorophenyl)-N-[4-(pyridin-2-yloxy)-3-sulfamoylphenyl]acetamide

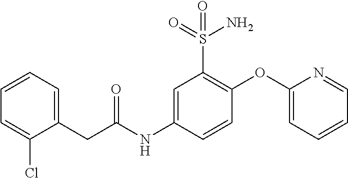 [SMILES: NS(=O)(=O)c1cc(NC(=O)Cc2ccccc2Cl)ccc1Oc1ccccn1]

According to general procedures GP1.1, GP2.2, GP3.2 and GP4, 2-chloro-N-(2,4-dimethoxybenzyl)-5-nitrobenzenesulfonamide (500 mg, 1.29 mmol), pyridin-2-ol (123 mg, 1.29 mmol) and (2-chlorophenyl)acetic acid (239 mg, 1.40 mmol) were converted without purification of intermediates to 2-(2-chlorophenyl)-N-[4-(pyridin-2-yloxy)-3-sulfamoylphenyl]acetamide (among other isomers) and were purified at the end twice by preparative HPLC (Waters XBrigde C18 5μ 100×30 mm, acetonitrile/water+0.1% formic acid), (27 mg, 0.646 mmol, 5% yield over 4 steps, 98% purity).

LC-MS (Method A): Rt=0.99 min

MS (ESIpos): m/z=418 (M+H)⁺

¹H-NMR (400 MHz, DMSO-d₆) δ [ppm]: 3.85 (s, 2H), 7.11 (ddd, 1H), 7.15 (d, 1H), 7.23 (d, 1H), 7.26-7.34 (m, 4H), 7.39-7.47 (m, 2H), 7.79 (dd, 1H), 7.83 (ddd, 1H), 8.07 (ddd, 1H), 8.17 (d, 1H), 10.49 (s, 1H).

### Example 014

### 2-(2-Chlorophenyl)-N-(4-phenoxy-3-sulfamoylphenyl)acetamide

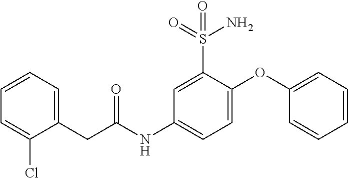 [SMILES: NS(=O)(=O)c1cc(NC(=O)Cc2ccccc2Cl)ccc1Oc1ccccc1]

According to general procedures GP1.1, GP2.2, GP3.2 and GP4, 2-chloro-N-(2,4-dimethoxybenzyl)-5-nitrobenzenesulfonamide (500 mg, 1.29 mmol), phenol (127 mg, 1.29 mmol) and (2-chlorophenyl)acetic acid (240 mg, 1.41 mmol) were converted without purification of intermediates to 2-(2-chlorophenyl)-N-(4-phenoxy-3-sulfamoylphenyl)acetamide and were purified at the end by preparative HPLC (Waters XBrigde C18 5μ 100×30 mm, acetonitrile/water+0.1% formic acid) (67 mg, 0.161 mmol, 12% yield over 4 steps, 98% purity).

LC-MS (Method A): Rt=1.12 min

MS (ESIpos): m/z=417 (M+H)⁺

¹H-NMR (400 MHz, DMSO-d₆) δ [ppm]: 3.83 (s, 2H), 6.92 (d, 1H), 7.02-7.08 (m, 2H), 7.14 (tt, 1H), 7.26-7.35 (m, 4H), 7.35-7.47 (m, 4H), 7.74 (dd, 1H), 8.18 (d, 1H), 10.45 (s, 1H).

### Example 015

### 2-(2-Chlorophenyl)-N-[4-(3-cyanophenoxy)-3-sulfamoylphenyl]acetamide

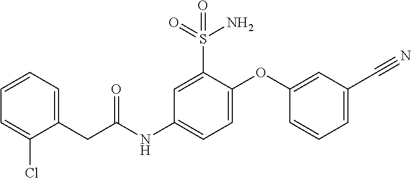 [SMILES: N#Cc1cccc(Oc2ccc(NC(=O)Cc3ccccc3Cl)cc2S(N)(=O)=O)c1]

According to general procedures GP1.1, GP2.2, GP3.2 and GP4, 2-chloro-N-(2,4-dimethoxybenzyl)-5-nitrobenzenesulfonamide (1.00 g, 2.59 mmol), 3-hydroxybenzonitrile (308 mg, 2.59 mmol) and (2-chlorophenyl)acetic acid (485 mg, 2.84 mmol) were converted without purification of intermediates to 2-(2-chlorophenyl)-N-[4-(3-cyanophenoxy)-3-sulfamoylphenyl]acetamide and were purified at the end by preparative HPLC (Chromatorex C-18 10 μm, 125×30 mm, acetonitrile/water+0.1% formic acid) (112 mg, 0.253 mmol, 10% yield over 4 steps, 98% purity).

LC-MS (Method A): Rt=1.09 min

MS (ESIpos): m/z=442 (M+H)⁺

¹H-NMR (400 MHz, DMSO-d₆) δ [ppm]: 3.85 (s, 2H), 7.11 (d, 1H), 7.27-7.35 (m, 3H), 7.37-7.48 (m, 5H), 7.53-7.60 (m, 2H), 7.81 (dd, 1H), 8.22 (d, 1H), 10.53 (s, 1H).

### Example 016

### 2-(2-Chlorophenyl)-N-{4-[3-(methylsulfonyl)phenoxy]-3-sulfamoylphenyl}acetamide

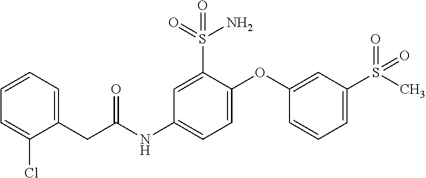 [SMILES: CS(=O)(=O)c1cccc(Oc2ccc(NC(=O)Cc3ccccc3Cl)cc2S(N)(=O)=O)c1]

According to general procedures GP1.1, GP2.2, GP3.2 and GP4, 2-chloro-N-(2,4-dimethoxybenzyl)-5-nitrobenzenesulfonamide (1.00 g, 2.59 mmol), 3-(methylsulfonyl)phenol (445 mg, 2.59 mmol) and (2-chlorophenyl)acetic acid (485 mg, 2.84 mmol) were converted without purification of intermediates to 2-(2-chlorophenyl)-N-{4-[3-(methylsulfonyl)phenoxy]-3-sulfamoylphenyl}acetamide and were purified at the end by preparative HPLC (Chromatorex C-18 10 μm, 125×30 mm, acetonitrile/water+0.1% formic acid) (50 mg, 0.101 mmol, 4% yield over 4 steps, 98% purity).

LC-MS (Method A): Rt=1.02 min

MS (ESIpos): m/z=495 (M+H)⁺

¹H-NMR (400 MHz, DMSO-d₆) δ [ppm]: 3.21 (s, 3H), 3.85 (s, 2H), 7.13 (d, 1H), 7.26-7.35 (m, 3H), 7.40-7.47 (m, 4H), 7.52-7.55 (m, 1H), 7.61-7.68 (m, 2H), 7.82 (dd, 1H), 8.23 (d, 1H), 10.53 (s, 1H).

### Example 017

### 3-(4-{[(2-Chlorophenyl)acetyl]amino}-2-sulfamoylphenoxy)benzamide

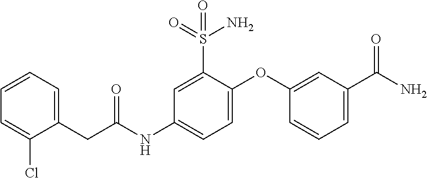 [SMILES: NC(=O)c1cccc(Oc2ccc(NC(=O)Cc3ccccc3Cl)cc2S(N)(=O)=O)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), 3-hydroxybenzamide (177 mg, 1.29 mmol) and (2-chlorophenyl)acetic acid (240 mg, 1.41 mmol) were converted without purification of intermediates to 3-(4-{[(2-chlorophenyl)acetyl]amino}-2-sulfamoylphenoxy)benzamide and were purified at the end by preparative HPLC (Waters XBrigde C18 5μ 100×30 mm, acetonitrile/water+0.1% formic acid) (47 mg, 0.102 mmol, 8% yield over 4 steps, 98% purity).

LC-MS (Method A): Rt=0.92 min

MS (ESIpos): m/z=460 (M+H)⁺

¹H-NMR (400 MHz, DMSO-d₆) δ [ppm]: 3.84 (s, 2H), 6.98 (d, 1H), 7.18 (ddd, 1H), 7.26-7.37 (m, 4H), 7.38-7.49 (m, 4H), 7.51-7.56 (m, 1H), 7.63-7-67 (m, 1H), 7.76 (dd, 1H), 7.98 (s, 1H), 8.21 (d, 1H), 10.48 (s, 1H).

### Example 018

### 2-(2-Chlorophenyl)-N-[4-(3-methylphenoxy)-3-sulfamoylphenyl]acetamide

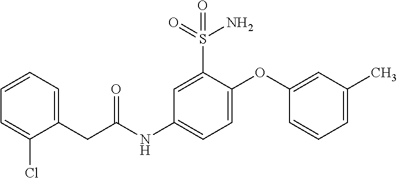 [SMILES: Cc1cccc(Oc2ccc(NC(=O)Cc3ccccc3Cl)cc2S(N)(=O)=O)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), m-cresol (140 mg, 1.29 mmol) and (2-chlorophenyl)acetic acid (242 mg, 1.42 mmol) were converted without purification of intermediates to 2-(2-chlorophenyl)-N-[4-(3-methylphenoxy)-3-sulfamoylphenyl]acetamide and were purified at the end by preparative HPLC (Waters XBrigde C18 5μ 100×30 mm, acetonitrile/water+0.1% formic acid) (41 mg, 0.095 mmol, 7% yield over 4 steps, 98% purity).

LC-MS (Method A): Rt=1.19 min

MS (ESIpos): m/z=431 (M+H)⁺

¹H-NMR (400 MHz, DMSO-d₆) δ [ppm]: 2.28 (s, 3H), 3.83 (s, 2H), 6.81-6.89 (m, 2H), 6.92 (d, 1H), 6.96 (d, 1H), 7.23-7.34 (m, 5H), 7.39-7.46 (m, 2H), 7.73 (dd, 1H), 8.17 (d, 1H), 10.44 (s, 1H).

### Example 019

### 2-(2-Chlorophenyl)-N-[4-(pyrimidin-5-yloxy)-3-sulfamoylphenyl]acetamide

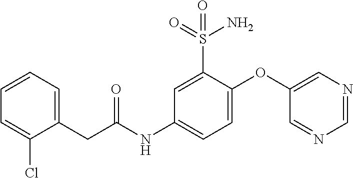 [SMILES: NS(=O)(=O)c1cc(NC(=O)Cc2ccccc2Cl)ccc1Oc1cncnc1]

According to general procedures GP1.1, GP2.2, GP3.2 and GP4, 2-chloro-N-(2,4-dimethoxybenzyl)-5-nitrobenzenesulfonamide (500 mg, 1.29 mmol), pyrimidin-5-ol (124 mg, 1.29 mmol) and (2-chlorophenyl)acetic acid (242 mg, 1.42 mmol) were converted without purification of intermediates to 2-(2-chlorophenyl)-N-[4-(pyrimidin-5-yloxy)-3-sulfamoylphenyl]acetamide and were purified at the end by preparative HPLC (Chromatorex C-18 10 μm, 125×30 mm, acetonitrile/water+0.1% formic acid) followed by another preparative HPLC (Waters XBrigde C18 5μ 100×30 mm, acetonitrile/water+0.1% formic acid) (2 mg, 0.00477 mmol, 0.4% yield over 4 steps, 98% purity).

LC-MS (Method A): Rt=0.89 min

MS (ESIpos): m/z=419 (M+H)⁺

¹H-NMR (400 MHz, DMSO-d₆) δ [ppm]: 3.85 (m, 2H), 7.24 (d, 1H), 7.28-7.34 (m, 2H), 7.39-7.46 (m, 2H), 7.49 (s, 2H), 7.82 (dd, 1H), 8.23 (d, 1H), 8.52 (s, 2H), 8.95 (s, 1H), 10.56 (s, 1H).

### Example 020

### 2-(2-Chlorophenyl)-N-{3-sulfamoyl-4-[3-(4H-1,2,4-triazol-4-yl)phenoxy]phenyl}acetamide

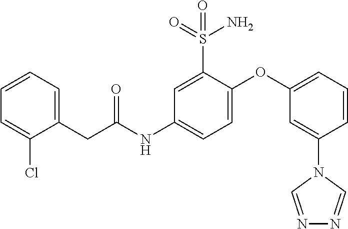 [SMILES: NS(=O)(=O)c1cc(NC(=O)Cc2ccccc2Cl)ccc1Oc1cccc(-n2cnnc2)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), 3-(4H-1,2,4-triazol-4-yl)phenol (208 mg, 1.29 mmol) and (2-chlorophenyl)acetic acid (242 mg, 1.42 mmol) were converted without purification of intermediates to 2-(2-chlorophenyl)-N-{3-sulfamoyl-4-[3-(4H-1,2,4-triazol-4-yl)phenoxy]phenyl}acetamide and were purified at the end by preparative HPLC (Waters XBrigde C18 5μ 100×30 mm, acetonitrile/water+0.1% formic acid) (17 mg, 0.0351 mmol, 3% yield over 4 steps, 98% purity).

LC-MS (Method A): Rt=0.92 min

MS (ESIpos): m/z=484 (M+H)⁺

¹H-NMR (400 MHz, DMSO-d₆) δ [ppm]: 3.84 (s, 2H), 6.99 (ddd, 1H), 7.09 (d, 1H), 7.27-7.34 (m, 2H), 7.36 (s, 2H), 7.40-7.46 (m, 2H), 7.46-7.50 (m, 1H), 7.51-7.57 (m, 2H), 7.79 (dd, 1H), 8.22 (d, 1H), 9.13 (s, 2H), 10.50 (s, 1H).

### Example 021

### 2-(2-Chlorophenyl)-N-{3-sulfamoyl-4-[3-(1H-tetrazol-5-yl)phenoxy]phenyl}acetamide

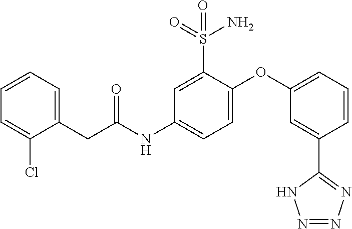 [SMILES: NS(=O)(=O)c1cc(NC(=O)Cc2ccccc2Cl)ccc1Oc1cccc(-c2nnn[nH]2)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), 3-(1H-tetrazol-5-yl)phenol (210 mg, 1.29 mmol) and (2-chlorophenyl)acetic acid (242 mg, 1.42 mmol) were converted without purification of intermediates to 2-(2-chlorophenyl)-N-{3-sulfamoyl-4-[3-(1H-tetrazol-5-yl)phenoxy]phenyl}acetamide and were purified at the end by preparative HPLC (Waters XBrigde C18 5μ 100×30 mm, acetonitrile/water+0.1% formic acid) (20 mg, 0.0412 mmol, 3% yield over 4 steps, 95% purity).

LC-MS (Method A): Rt=0.98 min

MS (ESIpos): m/z=485 (M+H)⁺

¹H-NMR (400 MHz, DMSO-d₆) δ [ppm]: 3.85 (s, 2H), 7.09 (d, 1H), 7.23 (ddd, 1H), 7.27-7.35 (m, 2H), 7.37-7.47 (m, 4H), 7.60 (t, 1H), 7.69 (t, 1H), 7.78-7.82 (m, 2H), 8.23 (d, 1H), 10.51 (s, 1H).

### Example 022

### 2-(2-Chlorophenyl)-N-[4-(3-methoxyphenoxy)-3-sulfamoylphenyl]acetamide

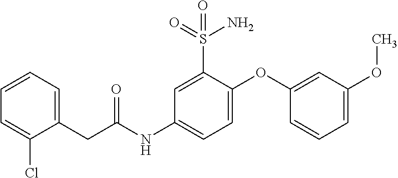 [SMILES: COc1cccc(Oc2ccc(NC(=O)Cc3ccccc3Cl)cc2S(N)(=O)=O)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), 3-methoxyphenol (160 mg, 1.29 mmol) and (2-chlorophenyl)acetic acid (242 mg, 1.42 mmol) were converted without purification of intermediates to 2-(2-chlorophenyl)-N-[4-(3-methoxyphenoxy)-3-sulfamoylphenyl]acetamide and were purified at the end by preparative HPLC (Waters XBrigde C18 5μ 100×30 mm, acetonitrile/water+0.1% formic acid) (42 mg, 0.0940 mmol, 7% yield over 4 steps, 97% purity).

LC-MS (Method A): Rt=1.13 min

MS (ESIpos): m/z=447 (M+H)⁺

¹H-NMR (400 MHz, DMSO-d₆) δ [ppm]: 3.75 (s, 3H), 3.85 (s, 2H), 6.60 (ddd, 1H), 6.67 (t, 1H), 6.74 (ddd, 1H), 6.99 (d, 1H), 7.25-7.36 (m, 5H), 7.41-7.49 (m, 2H), 7.76 (dd, 1H), 8.20 (d, 1H), 10.48 (s, 1H).

### Example 023

### 2-(2-Chlorophenyl)-N-[4-(4-methoxyphenoxy)-3-sulfamoylphenyl]acetamide

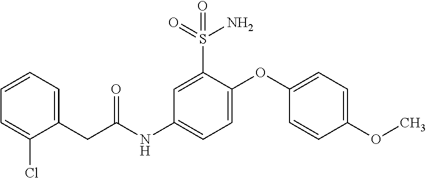 [SMILES: COc1ccc(Oc2ccc(NC(=O)Cc3ccccc3Cl)cc2S(N)(=O)=O)cc1]

According to general procedures GP1.1, GP2.2, GP3.2 and GP4, 2-chloro-N-(2,4-dimethoxybenzyl)-5-nitrobenzenesulfonamide (500 mg, 1.29 mmol), 4-methoxyphenol (160 mg, 1.29 mmol) and (2-chlorophenyl)acetic acid (242 mg, 1.42 mmol) were converted without purification of intermediates to 2-(2-chlorophenyl)-N-[4-(4-methoxyphenoxy)-3-sulfamoylphenyl]acetamide and were purified at the end by preparative HPLC (Waters XBrigde C18 5μ 100×30 mm, acetonitrile/water+0.1% formic acid) (38 mg, 0.0850 mmol, 7% yield over 4 steps, 98% purity).

LC-MS (Method A): Rt=1.12 min

MS (ESIpos): m/z=447 (M+H)⁺

¹H-NMR (400 MHz, DMSO-d₆) δ [ppm]: 3.76 (s, 3H), 3.83 (s, 2H), 6.83 (d, 1H), 6.95-7.07 (m, 4H), 7.28-7.35 (m, 4H), 7.39-7.48 (m, 2H), 7.71 (dd, 1H), 8.17 (d, 1H), 10.43 (s, 1H).

### Example 024

### 2-(2-Chlorophenyl)-N-{4-[3-(difluoromethoxy)phenoxy]-3-sulfamoylphenyl}acetamide

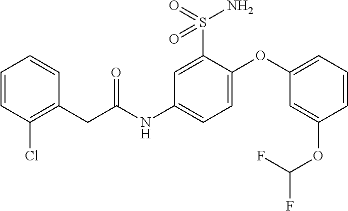 [SMILES: NS(=O)(=O)c1cc(NC(=O)Cc2ccccc2Cl)ccc1Oc1cccc(OC(F)F)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), 3-(difluoromethoxy)phenol (207 mg, 1.29 mmol) and (2-chlorophenyl)acetic acid (242 mg, 1.42 mmol) were converted without purification of intermediates to 2-(2-chlorophenyl)-N-{4-[3-(difluoromethoxy)phenoxy]-3-sulfamoylphenyl}acetamide and were purified at the end twice by preparative HPLC (Waters XBrigde C18 5μ 100×30 mm, acetonitrile/water+0.1% formic acid) (29 mg, 0.0601 mmol, 5% yield over 4 steps, 97% purity).

LC-MS (Method A): Rt=1.18 min

MS (ESIneg): m/z=481 (M−H)⁺

¹H-NMR (400 MHz, DMSO-d₆) δ [ppm]: 3.85 (s, 2H), 6.87 (dd, 1H), 6.90 (t, 1H), 6.93-6.96 (dd, 1H), 7.07 (d, 1H), 7.27 (t, 1H), 7.29-7.35 (m, 2H), 7.38 (s, 2H), 7.40-7.48 (m, 3H), 7.80 (dd, 1H), 8.22 (d, 1H), 10.52 (s, 1H).

### Example 025

### 2-(2-Chlorophenyl)-N-[4-(3,4-dicyanophenoxy)-3-sulfamoylphenyl]acetamide

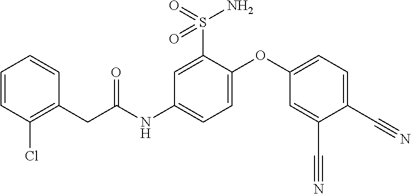 [SMILES: N#Cc1ccc(Oc2ccc(NC(=O)Cc3ccccc3Cl)cc2S(N)(=O)=O)cc1C#N]

According to general procedures GP1.1, GP2.2, GP3.2 and GP4, 2-chloro-N-(2,4-dimethoxybenzyl)-5-nitrobenzenesulfonamide (500 mg, 1.29 mmol), 4-hydroxyphthalonitrile (186 mg, 1.29 mmol) and (2-chlorophenyl)acetic acid (242 mg, 1.42 mmol) were converted without purification of intermediates to 2-(2-chlorophenyl)-N-[4-(3,4-dicyanophenoxy)-3-sulfamoylphenyl]acetamide and were purified at the end by preparative HPLC (Waters XBrigde C18 5μ 100×30 mm, acetonitrile/water+0.1% formic acid) (18 mg, 0.0386 mmol, 3% yield over 4 steps, 98% purity).

LC-MS (Method A): Rt=1.09 min

MS (ESIneg): m/z=465 (M−H)⁺

¹H-NMR (400 MHz, DMSO-d₆) δ [ppm]: 3.81 (s, 2H), 6.89 (d, 1H), 7.28-7.35 (m, 2H), 7.40-7.48 (m, 3H), 7.55 (s, 1H), 7.63 (dd, 1H), 7.94 (d, 1H), 8.15 (s, 1H), 10.27 (s, 1H), 11.05 (s, 2H).

### Example 026

### 2-(2-Chlorophenyl)-N-{4-[3-(morpholin-4-yl)phenoxy]-3-sulfamoylphenyl}acetamide

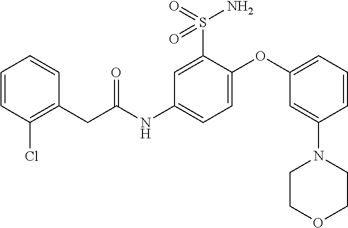 [SMILES: NS(=O)(=O)c1cc(NC(=O)Cc2ccccc2Cl)ccc1Oc1cccc(N2CCOCC2)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), 3-(morpholin-4-yl)phenol (232 mg, 1.29 mmol) and (2-chlorophenyl)acetic acid (242 mg, 1.42 mmol) were converted without purification of intermediates to 2-(2-chlorophenyl)-N-{4-[3-(morpholin-4-yl)phenoxy]-3-sulfamoylphenyl}acetamide and were purified at the end twice by preparative HPLC (Waters XBrigde C18 5μ 100×30 mm, acetonitrile/water+0.1% formic acid) (18 mg, 0.0386 mmol, 3% yield over 4 steps, 98% purity).

LC-MS (Method A): Rt=1.11 min

MS (ESIpos): m/z=502 (M+H)⁺

¹H-NMR (400 MHz, DMSO-d₆) δ [ppm]: 3.07-3.13 (m, 4H), 3.69-3.75 (m, 4H), 3.84 (s, 2H), 6.46 (dd, 1H), 6.69 (t, 1H), 6.76 (dd, 1H), 6.94 (d, 1H), 7.22 (t, 1H), 7.26-7.35 (m, 4H), 7.41-7.47 (m, 2H), 7.73 (dd, 1H), 8.18 (d, 1H), 10.45 (s, 1H).

### Example 027

### 2-(2-Chlorophenyl)-N-[4-(3-{4-[(2-chlorophenyl)acetyl]piperazin-1-yl}phenoxy)-3-sulfamoylphenyl]acetamide

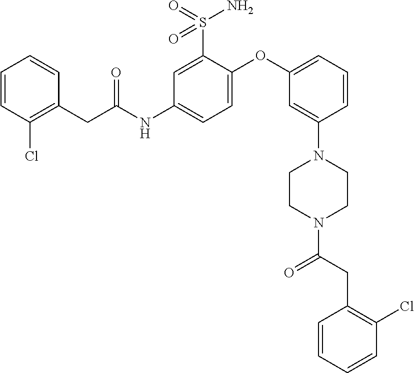 [SMILES: NS(=O)(=O)c1cc(NC(=O)Cc2ccccc2Cl)ccc1Oc1cccc(N2CCN(C(=O)Cc3ccccc3Cl)CC2)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), 3-(piperazin-1-yl)phenol (230 mg, 1.29 mmol) and (2-chlorophenyl)acetic acid (242 mg, 1.42 mmol) were converted without purification of intermediates to 2-(2-chlorophenyl)-N-[4-(3-{4-[(2-chlorophenyl)acetyl]piperazin-1-yl}phenoxy)-3-sulfamoylphenyl]acetamide and were purified at the end by preparative HPLC (Waters XBrigde C18 5μ 100×30 mm, acetonitrile/water+0.1% formic acid) (16 mg, 0.0245 mmol, 2% yield over 4 steps, 95% purity). The monoacylated compound could not be isolated.

LC-MS (Method A): Rt=1.26 min

MS (ESIneg): m/z=651 (M−H)⁺

¹H-NMR (400 MHz, DMSO-d₆) δ [ppm]: 3.11-3.22 (m, 4H), 3.59-3.72 (m, 4H), 3.84 (s, 2H), 3.86 (s, 2H), 6.47 (dd, 1H), 6.71 (t, 1H), 6.76 (dd, 1H), 6.95 (d, 1H), 7.23 (t, 1H), 7.26-7.35 (m, 7H), 7.41-7.48 (m, 3H), 7.74 (dd, 1H), 8.18 (d, 1H), 10.46 (s, 1H).

### Example 028

### 2-(2-Chlorophenyl)-N-[4-(pyridin-3-yloxy)-3-sulfamoylphenyl]acetamide

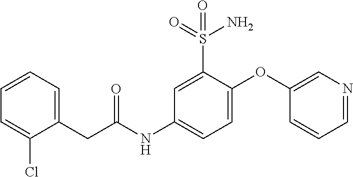 [SMILES: NS(=O)(=O)c1cc(NC(=O)Cc2ccccc2Cl)ccc1Oc1cccnc1]

According to general procedures GP1.1, GP2.2, GP3.2 and GP4, 2-chloro-N-(2,4-dimethoxybenzyl)-5-nitrobenzenesulfonamide (500 mg, 1.29 mmol), pyridin-3-ol (123 mg, 1.29 mmol) and (2-chlorophenyl)acetic acid (242 mg, 1.42 mmol) were converted without purification of intermediates to 2-(2-chlorophenyl)-N-[4-(pyridin-3-yloxy)-3-sulfamoylphenyl]acetamide and were purified at the end by preparative HPLC (Waters XBrigde C18 5μ 100×30 mm, acetonitrile/water+0.1% formic acid) (45 mg, 0.108 mmol, 8% yield over 4 steps, 95% purity).

LC-MS (Method A): Rt=0.89 min

MS (ESIneg): m/z=416 (M−H)⁺

¹H-NMR (400 MHz, DMSO-d₆) δ [ppm]: 3.85 (s, 2H), 7.07 (d, 1H), 7.26-7.46 (m, 8H), 7.80 (dd, 1H), 8.22 (d, 1H), 8.35 (dd, 1H), 8.38 (d, 1H), 10.52 (s, 1H).

### Example 029

### 2-(2-Chlorophenyl)-N-{4-[(5-chloropyridin-3-yl)oxy]-3-sulfamoylphenyl}acetamide

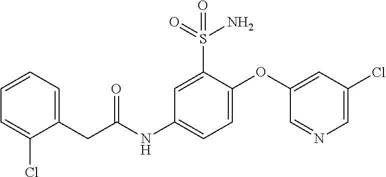 [SMILES: NS(=O)(=O)c1cc(NC(=O)Cc2ccccc2Cl)ccc1Oc1cncc(Cl)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), 5-chloropyridin-3-ol (167 mg, 1.29 mmol) and (2-chlorophenyl)acetic acid (242 mg, 1.42 mmol) were converted without purification of intermediates to 2-(2-chlorophenyl)-N-{4-[(5-chloropyridin-3-yl)oxy]-3-sulfamoylphenyl}acetamide and were purified at the end by preparative HPLC (Waters XBrigde C18 5μ 100×30 mm, acetonitrile/water+0.1% formic acid) followed by another preparative HPLC (Waters XBrigde C18 5μ 100×30 mm, acetonitrile/water+0.2% aqueous ammonia (32%)) (11.2 mg, 0.0248 mmol, 2% yield over 4 steps, 95% purity).

LC-MS (Method B): Rt=0.99 min

MS (ESIpos): m/z=452 (M+H)⁺

¹H-NMR (400 MHz, METHANOL-d₄) δ [ppm]: 3.90 (s, 2H), 7.14 (d, 1H), 7.25-7.33 (m, 2H), 7.41 (s, 2H), 7.51 (t, 1H), 7.87 (dd, 1H), 8.25 (d, 1H), 8.29 (d, 1H), 8.32 (d, 1H).

### Example 030

### 2-(2-Chlorophenyl)-N-[(4-cyanophenoxy)-3-sulfamoylphenyl]acetamide

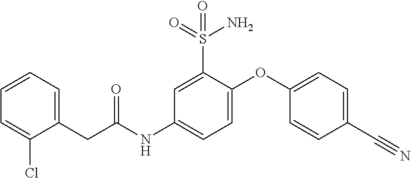 [SMILES: N#Cc1ccc(Oc2ccc(NC(=O)Cc3ccccc3Cl)cc2S(N)(=O)=O)cc1]

According to general procedures GP1.1, GP2.2, GP3.2 and GP4, 2-chloro-N-(2,4-dimethoxybenzyl)-5-nitrobenzenesulfonamide (500 mg, 1.29 mmol), 4-hydroxybenzonitrile (154 mg, 1.29 mmol) and (2-chlorophenyl)acetic acid (242 mg, 1.42 mmol) were converted without purification of intermediates to 2-(2-chlorophenyl)-N-[4-(4-cyanophenoxy)-3-sulfamoylphenyl]acetamide and were purified at the end by preparative HPLC (Waters XBrigde C18 5μ 100×30 mm, acetonitrile/water+0.1% formic acid) (10.6 mg, 0.0240 mmol, 2% yield over 4 steps, 97% purity).

LC-MS (Method A): Rt=1.06 min

MS (ESIpos): m/z=442 (M+H)⁺

¹H-NMR (400 MHz, DMSO-d₆) δ [ppm]: 3.80 (s, 2H), 6.87 (d, 1H), 7.19 (d, 2H), 7.28-7.35 (m, 2H), 7.40-7.48 (m, 2H), 7.60 (dd, 1H), 7.65 (d, 2H), 8.11 (d, 1H), 10.24 (s, 1H), 10.80 (s, 2H).

### Example 031

### 2-(2-Chlorophenyl)-N-{4-[3-(difluoromethyl)phenoxy]-3-sulfamoylphenyl}acetamide

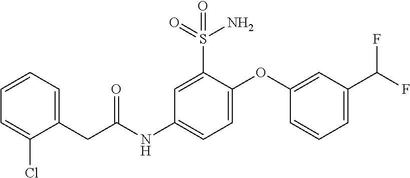 [SMILES: NS(=O)(=O)c1cc(NC(=O)Cc2ccccc2Cl)ccc1Oc1cccc(C(F)F)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), 3-(difluoromethyl)phenol (186 mg, 1.29 mmol) and (2-chlorophenyl)acetic acid (242 mg, 1.42 mmol) were converted without purification of intermediates to 2-(2-chlorophenyl)-N-{4-[3-(difluoromethyl)phenoxy]-3-sulfamoylphenyl}acetamide and were purified at the end by preparative HPLC (Waters XBrigde C18 5μ 100×30 mm, acetonitrile/water+0.1% formic acid) (22.5 mg, 0.0482 mmol, 4% yield over 4 steps, 97% purity).

LC-MS (Method A): Rt=1.17 min

MS (ESIpos): m/z=467 (M+H)⁺

¹H-NMR (400 MHz, DICHLOROMETHANE-d₂) δ [ppm]: 3.89 (s, 2H), 5.20 (s, 2H), 6.69 (t, 1H), 6.96 (d, 1H), 7.20-7.61 (m, 8H), 7.86 (dd, 1H), 7.95 (d, 1H).

### Example 032

### 2-(2-Chlorophenyl)-N-[4-(2-methoxyphenoxy)-3-sulfamoylphenyl]acetamide

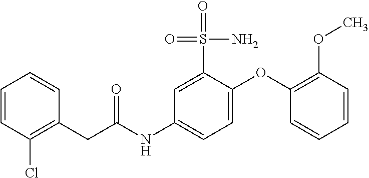 [SMILES: COc1ccccc1Oc1ccc(NC(=O)Cc2ccccc2Cl)cc1S(N)(=O)=O]

According to general procedures GP1.1, GP2.2, GP3.2 and GP4, 2-chloro-N-(2,4-dimethoxybenzyl)-5-nitrobenzenesulfonamide (500 mg, 1.29 mmol), 2-methoxyphenol (160 mg, 1.29 mmol) and (2-chlorophenyl)acetic acid (331 mg, 1.94 mmol) were converted without purification of intermediates to 2-(2-chlorophenyl)-N-[4-(2-methoxyphenoxy)-3-sulfamoylphenyl]acetamide and were purified at the end by preparative HPLC (Waters XBrigde C18 5μ 100×30 mm, acetonitrile/water+0.1% formic acid) (140 mg, 0.0313 mmol, 24% yield over 4 steps, 99% purity).

LC-MS (Method A): Rt=1.13 min

MS (ESIpos): m/z=447 (M+H)⁺

¹H-NMR (400 MHz, DICHLOROMETHANE-d₂) δ [ppm]: 3.79 (s, 3H), 3.85 (s, 2H), 5.39 (s, 2H), 6.79 (d, 1H), 7.04 (td, 1H), 7.08 (dd, 1H), 7.21 (dd, 1H), 7.25-7.38 (m, 4H), 7.40-7.50 (m, 2H), 7.73 (dd, 1H), 7.84 (d, 1H).

### Example 033

### 2-(2-Chlorophenyl)-N-[4-(3,5-dicyanophenoxy)-3-sulfamoylphenyl]acetamide

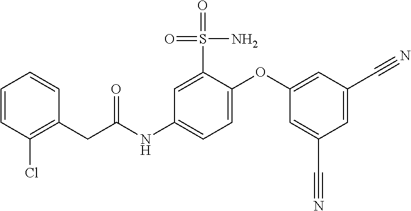 [SMILES: N#Cc1cc(C#N)cc(Oc2ccc(NC(=O)Cc3ccccc3Cl)cc2S(N)(=O)=O)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), 5-hydroxyisophthalonitrile (186 mg, 1.29 mmol) and (2-chlorophenyl)acetic acid (330 mg, 1.94 mmol) were converted without purification of intermediates to 2-(2-chlorophenyl)-N-[4-(3,5-dicyanophenoxy)-3-sulfamoylphenyl]acetamide and were purified at the end by preparative HPLC (Waters XBrigde C18 5μ 100×30 mm, acetonitrile/water+0.1% formic acid) (65 mg, 0.139 mmol, 11% yield over 4 steps, 97% purity).

LC-MS (Method A): Rt=1.08 min

MS (ESIpos): m/z=467 (M+H)⁺

¹H-NMR (400 MHz, DMSO-d₆) δ [ppm]: 3.78 (s, 2H), 6.88 (d, 1H), 7.26-7.33 (m, 2H), 7.38-7.45 (m, 2H), 7.62 (dd, 1H), 7.67 (d, 2H), 8.02 (s, 1H), 8.08 (d, 1H), 10.23 (s, 1H), 10.85 (s, 2H).

### Example 034

### 2-(2-Chlorophenyl)-N-[4-(5-cyano-2-methoxyphenoxy)-3-sulfamoylphenyl]acetamide

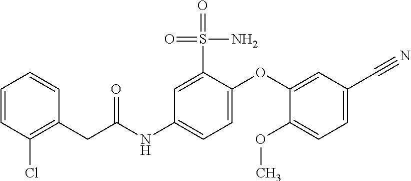 [SMILES: COc1ccc(C#N)cc1Oc1ccc(NC(=O)Cc2ccccc2Cl)cc1S(N)(=O)=O]

According to general procedures GP1.1, GP2.2, GP3.2 and GP4, 2-chloro-N-(2,4-dimethoxybenzyl)-5-nitrobenzenesulfonamide (500 mg, 1.29 mmol), 3-hydroxy-4-methoxybenzonitrile (92 mg, 1.29 mmol) and (2-chlorophenyl)acetic acid (330 mg, 1.94 mmol) were converted without purification of intermediates to 2-(2-chlorophenyl)-N-[4-(5-cyano-2-methoxyphenoxy)-3-sulfamoylphenyl]acetamide and were purified at the end by preparative HPLC (Waters XBrigde C18 5μ 100×30 mm, acetonitrile/water+0.1% formic acid) (132 mg, 0.280 mmol, 22% yield over 4 steps, 99% purity).

LC-MS (Method A): Rt=1.07 min

MS (ESIpos): m/z=472 (M+H)⁺

¹H-NMR (400 MHz, DMSO-d₆) δ [ppm]: 3.82 (s, 2H), 3.84 (s, 3H), 6.75 (d, 1H), 7.25 (s, 2H), 7.28-7.32 (m, 2H), 7.34 (d, 1H), 7.39-7.46 (m, 2H), 7.48 (d, 1H), 7.68 (dd, 1H), 7.72 (dd, 1H), 8.16 (d, 1H), 10.42 (s, 1H).

### Example 035

### 2-(2-Chlorophenyl)-N-{4-[(2,5-dichloropyridin-3-yl)oxy]-3-sulfamoylphenyl}acetamide

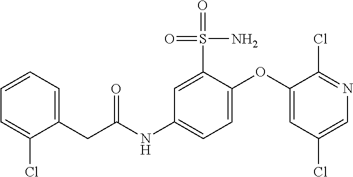 [SMILES: NS(=O)(=O)c1cc(NC(=O)Cc2ccccc2Cl)ccc1Oc1cc(Cl)cnc1Cl]

According to general procedures GP1.1, GP2.2, GP3.2 and GP4, 2-chloro-N-(2,4-dimethoxybenzyl)-5-nitrobenzenesulfonamide (500 mg, 1.29 mmol), 2,5-dichloropyridin-3-ol (212 mg, 1.29 mmol) and (2-chlorophenyl)acetic acid (330 mg, 1.94 mmol) were converted without purification of intermediates to 2-(2-chlorophenyl)-N-{4-[(2,5-dichloropyridin-3-yl)oxy]-3-sulfamoylphenyl}acetamide and were purified at the end by preparative HPLC (Waters XBrigde C18 5μ 100×30 mm, acetonitrile/water+0.1% formic acid) (32 mg, 0.0657 mmol, 5% yield over 4 steps, 90% purity).

LC-MS (Method A): Rt=1.16 min

MS (ESIpos): m/z=487/488 (M+H)⁺

¹H-NMR (400 MHz, DMSO-d₆) δ [ppm]: 3.77 (s, 2H), 6.91 (d, 1H), 7.25-7.33 (m, 2H), 7.36-7.46 (m, 2H), 7.63 (dd, 1H), 7.84 (d, 1H), 7.96 (d, 1H), 8.24 (s, 1H), 9.92 (s, 1H), 10.19 (s, 1H), 10.71 (s, 1H).

### Example 036

### 2-(2-Chlorophenyl)-N-{4-[(5,6-dichloropyridin-3-yl)oxy]-3-sulfamoylphenyl}acetamide

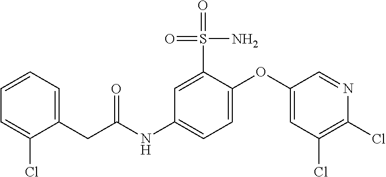 [SMILES: NS(=O)(=O)c1cc(NC(=O)Cc2ccccc2Cl)ccc1Oc1cnc(Cl)c(Cl)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), 5,6-dichloropyridin-3-ol (212 mg, 1.29 mmol) and (2-chlorophenyl)acetic acid (330 mg, 1.94 mmol) were converted without purification of intermediates to 2-(2-chlorophenyl)-N-{4-[(5,6-dichloropyridin-3-yl)oxy]-3-sulfamoylphenyl}acetamide and were purified at the end by preparative HPLC (Waters XBrigde C18 5μ 100×30 mm, acetonitrile/water+0.1% formic acid) (98 mg, 0.154 mmol, 12% yield over 4 steps, 93% purity).

LC-MS (Method A): Rt=1.24 min

MS (ESIpos): m/z=487/488 (M+H)⁺

¹H-NMR (400 MHz, DMSO-d₆) δ [ppm]: 3.83 (s, 2H), 6.89 (d, 1H), 7.06 (d, 1H), 7.25-7.47 (m, 6H), 7.63 (d, 1H), 7.75 (dd, 1H), 8.22 (d, 1H), 10.50 (s, 1H).

### Example 037

### 3-(4-{[(2-Chlorophenyl)acetyl]amino}-2-sulfamoylphenoxy)-N-cyclopropylbenzamide

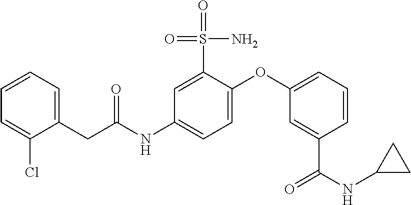 [SMILES: NS(=O)(=O)c1cc(NC(=O)Cc2ccccc2Cl)ccc1Oc1cccc(C(=O)NC2CC2)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), N-cyclopropyl-3-hydroxybenzamide (229 mg, 1.29 mmol) and (2-chlorophenyl)acetic acid (121 mg, 0.707 mmol) were converted without purification of intermediates to 3-(4-{[(2-chlorophenyl)acetyl]amino}-2-sulfamoylphenoxy)-N-cyclopropylbenzamide and were purified at the end by preparative HPLC (Waters XBrigde C18 5μ 100×30 mm, acetonitrile/water+0.1% formic acid) (9 mg, 0.0180 mmol, 1% yield over 4 steps, 97% purity).

LC-MS (Method A): Rt=1.02 min

MS (ESIpos): m/z=500 (M+H)⁺

¹H-NMR (400 MHz, DMSO-d₆) δ [ppm]: 0.53-0.59 (m, 2H), 0.66-0.72 (m, 2H), 2.79-2.87 (m, 1H), 3.85 (s, 2H), 6.98 (d, 1H), 7.17 (ddd, 1H), 7.28-7.35 (m, 2H), 7.38 (s, 2H), 7.41-7.48 (m, 3H), 7.52 (t, 1H), 7.59-7.63 (m, 1H), 7.77 (dd, 1H), 8.22 (d, 1H), 8.50 (d, 1H), 10.52 (s, 1H).

### Example 038

### 2-(2-chlorophenyl)-N-{4-[(3-chloropyridin-2-yl)oxy]-3-sulfamoylphenyl}acetamide

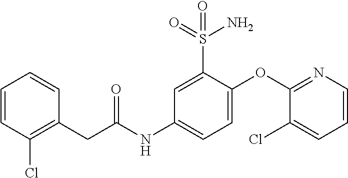 [SMILES: NS(=O)(=O)c1cc(NC(=O)Cc2ccccc2Cl)ccc1Oc1ncccc1Cl]

According to general procedures GP1.1, GP2.2, GP3.2 and GP4, 2-chloro-N-(2,4-dimethoxybenzyl)-5-nitrobenzenesulfonamide (1500 mg, 3.88 mmol), 3-chloropyridin-2-ol (502 mg, 3.88 mmol) and (2-chlorophenyl)acetic acid (646 mg, 3.79 mmol) were converted without purification of intermediates to 2-(2-chlorophenyl)-N-{4-[(3-chloropyridin-2-yl)oxy]-3-sulfamoylphenyl}acetamide (among other isomers) and were purified at the end by preparative HPLC (YMC—Triart C18 5μ 100×30 mm, acetonitrile/water+0.1% formic acid) followed by another preparative HPLC (Phenomenex Kinetex C18 5μ 100×30 mm, acetonitrile/water+0.1% trifluoroacetic acid) (1.8 mg, 0.00398 mmol, 0.1% yield over 4 steps, 97% purity).

LC-MS (Method D): Rt=2.12 min

MS (ESIpos): m/z=452 (M+H)⁺

¹H-NMR (500 MHz, DMSO-d₆) δ [ppm]: 3.87 (s, 2H), 7.14-7.21 (m, 3H), 7.25 (d, 1H), 7.29-7.35 (m, 2H), 7.41-7.48 (m, 2H), 7.82 (dd, 1H), 8.00-8.03 (m, 2H), 8.21 (d, 1H), 10.55 (s, 1H).

### Example 039

### 2-(2-chlorophenyl)-N-{4-[(4-chloropyridin-2-yl)oxy]-3-sulfamoylphenyl}acetamide

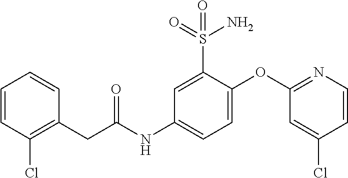 [SMILES: NS(=O)(=O)c1cc(NC(=O)Cc2ccccc2Cl)ccc1Oc1cc(Cl)ccn1]

According to general procedures GP1.1, GP2.2, GP3.2 and GP4, 2-chloro-N-(2,4-dimethoxybenzyl)-5-nitrobenzenesulfonamide (1500 mg, 3.88 mmol), 4-chloropyridin-2-ol (502 mg, 3.88 mmol) and (2-chlorophenyl)acetic acid (634 mg, 3.72 mmol) were converted without purification of intermediates to 2-(2-chlorophenyl)-N-{4-[(3-chloropyridin-2-yl)oxy]-3-sulfamoylphenyl}acetamide (among other isomers) and were purified at the end by preparative HPLC (YMC—Triart C18 5μ 100×30 mm, acetonitrile/water+0.1% formic acid) followed by another preparative HPLC (Phenomenex Kinetex C18 5μ 100×30 mm, acetonitrile/water+0.1% trifluoroacetic acid) (2.8 mg, 0.00619 mmol, 0.2% yield over 4 steps, 90% purity).

LC-MS (Method D): Rt=2.17 min

MS (ESIpos): m/z=452 (M+H)⁺

¹H-NMR (500 MHz, DMSO-d₆) δ [ppm]: 3.87 (s, 2H), 7.24 (dd, 1H), 7.29-7.38 (m, 6H), 7.40-7.49 (m, 2H), 7.82 (dd, 1H), 8.07 (d, 1H), 8.18 (d, 1H), 10.53 (s, 1H).

### Example 040

### 2-(2-Chlorophenyl)-N-{4-[(6-chloropyridin-2-yl)oxy]-3-sulfamoylphenyl}acetamide

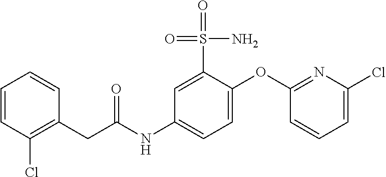 [SMILES: NS(=O)(=O)c1cc(NC(=O)Cc2ccccc2Cl)ccc1Oc1cccc(Cl)n1]

According to general procedures GP1.1, GP2.2, GP3.2 and GP4, 2-chloro-N-(2,4-dimethoxybenzyl)-5-nitrobenzenesulfonamide (1500 mg, 3.88 mmol), 6-chloropyridin-2-ol (502 mg, 3.88 mmol) and (2-chlorophenyl)acetic acid (413 mg, 2.42 mmol) were converted without purification of intermediates to 2-(2-chlorophenyl)-N-{4-[(6-chloropyridin-2-yl)oxy]-3-sulfamoylphenyl}acetamide (among other isomers) and were purified at the end by preparative HPLC (YMC—Triart C18 5μ 100×30 mm, acetonitrile/water+0.1% formic acid) (11.2 mg, 0.0248 mmol, 0.6% yield over 4 steps, 95% purity).

LC-MS (Method D): Rt=2.20 min

MS (ESIpos): m/z=452 (M+H)⁺

¹H-NMR (500 MHz, DMSO-d₆) δ [ppm]: 3.86 (s, 2H), 7.12 (d, 1H), 7.22 (d, 1H), 7.28-7.35 (m, 3H), 7.38 (s, 2H), 7.42-7.48 (m, 2H), 7.84 (dd, 1H), 7.89 (t, 1H), 8.20 (d, 1H), 10.55 (s, 1H).

### Example 041

### 2-(2-Chlorophenyl)-N-{4-[3-(1-methyl-4,5-dihydro-1H-imidazol-2-yl)phenoxy]-3-sulfamoylphenyl}acetamide

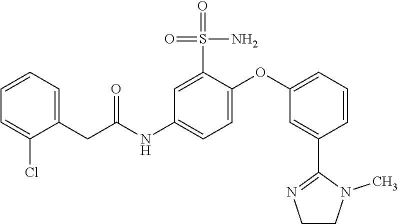 [SMILES: CN1CCN=C1c1cccc(Oc2ccc(NC(=O)Cc3ccccc3Cl)cc2S(N)(=O)=O)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), 3-(1-methyl-4,5-dihydro-1H-imidazol-2-yl)phenol (228 mg, 1.29 mmol) and (2-chlorophenyl)acetic acid (241 mg, 1.41 mmol) were converted without purification of intermediates to 2-(2-chlorophenyl)-N-{4-[3-(1-methyl-4,5-dihydro-1H-imidazol-2-yl)phenoxy]-3-sulfamoylphenyl}acetamide and were purified at the end by preparative HPLC (Waters XBrigde C18 5μ 100×30 mm, acetonitrile/water+0.1% formic acid) (5 mg, 0.100 mmol, 1% yield over 4 steps, 98% purity).

LC-MS (Method A): Rt=0.83 min

MS (ESIpos): m/z=499 (M+H)⁺

¹H-NMR (400 MHz, METHANOL-d₄) δ [ppm]: 3.10 (s, 3H), 3.92 (s, 2H), 3.94-4.12 (m, 4H), 7.12 (d, 1H), 7.23-7.27 (m, 1H), 7.28-7.35 (m, 2H), 7.39-7.48 (m, 4H), 7.65 (t, 1H), 7.91 (dd, 1H), 8.23 (d, 1H).

### Example 042

### 2-(2-Chlorophenyl)-N-{4-[4-(1H-imidazol-1-yl)phenoxy]-3-sulfamoylphenyl}acetamide

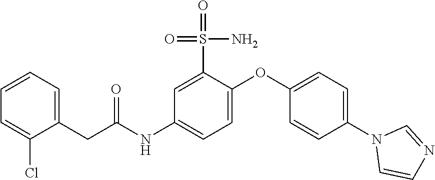 [SMILES: NS(=O)(=O)c1cc(NC(=O)Cc2ccccc2Cl)ccc1Oc1ccc(-n2ccnc2)cc1]

According to general procedures GP1.1, GP2.2, GP3.2 and GP4, 2-chloro-N-(2,4-dimethoxybenzyl)-5-nitrobenzenesulfonamide (500 mg, 1.29 mmol), 4-(1H-imidazol-1-yl)phenol (207 mg, 1.29 mmol) and (2-chlorophenyl)acetic acid (197 mg, 1.15 mmol) were converted without purification of intermediates to 2-(2-chlorophenyl)-N-{4-[4-(1H-imidazol-1-yl)phenoxy]-3-sulfamoylphenyl}acetamide and were purified at the end by preparative HPLC (Waters XBrigde C18 5μ 100×30 mm, acetonitrile/water+0.1% formic acid) (6 mg, 0.0124 mmol, 1% yield over 4 steps, 97% purity).

LC-MS (Method A): Rt=0.82 min

MS (ESIpos): m/z=483 (M+H)⁺

¹H-NMR (400 MHz, METHANOL-d₄) δ [ppm]: 3.91 (s, 2H), 7.06 (d, 1H), 7.24-7.34 (m, 5H), 7.40-7.45 (m, 2H), 7.61-7.65 (m, 2H), 7.69 (t, 1H), 7.85 (dd, 1H), 8.23 (d, 1H), 8.46 (s, 1H).

### Example 043

### 2-(2-Chlorophenyl)-N-{4-[4-(2-oxopyrrolidin-1-yl)phenoxy]-3-sulfamoylphenyl}acetamide

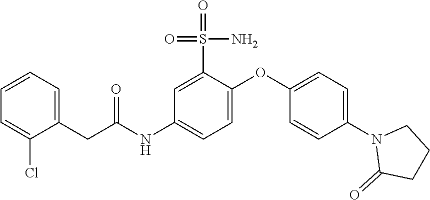 [SMILES: NS(=O)(=O)c1cc(NC(=O)Cc2ccccc2Cl)ccc1Oc1ccc(N2CCCC2=O)cc1]

According to general procedures GP1.1, GP2.2, GP3.2 and GP4, 2-chloro-N-(2,4-dimethoxybenzyl)-5-nitrobenzenesulfonamide (500 mg, 1.29 mmol), 1-(4-hydroxyphenyl)pyrrolidin-2-one (229 mg, 1.29 mmol) and (2-chlorophenyl)acetic acid (213 mg, 1.25 mmol) were converted without purification of intermediates to 2-(2-chlorophenyl)-N-{4-[4-(1H-imidazol-1-yl)phenoxy]-3-sulfamoylphenyl}acetamide and were purified at the end by preparative HPLC (Waters XBrigde C18 5μ 100×30 mm, acetonitrile/water+0.1% formic acid) (15 mg, 0.0300 mmol, 2% yield over 4 steps, 95% purity).

LC-MS (Method A): Rt=1.03 min

MS (ESIpos): m/z=500 (M+H)⁺

¹H-NMR (400 MHz, METHANOL-d₄) δ [ppm]: 2.19 (quin, 2H), 2.60 (t, 2H), 3.88 (s, 2H), 3.92 (t, 2H), 6.91 (d, 1H), 7.12-7.16 (m, 2H), 7.25-7.32 (m, 2H), 7.36-7.43 (m, 2H), 7.60-7.64 (m, 2H), 7.75 (dd, 1H), 8.18 (d, 1H).

### Example 044

### 2-(2-Chlorophenyl)-N-{4-[4-(morpholin-4-yl)phenoxy]-3-sulfamoylphenyl}acetamide

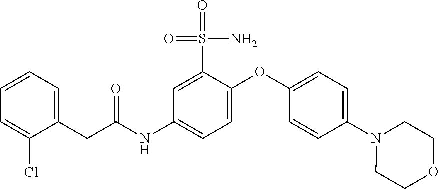 [SMILES: NS(=O)(=O)c1cc(NC(=O)Cc2ccccc2Cl)ccc1Oc1ccc(N2CCOCC2)cc1]

According to general procedures GP1.1, GP2.2, GP3.2 and GP4, 2-chloro-N-(2,4-dimethoxybenzyl)-5-nitrobenzenesulfonamide (500 mg, 1.29 mmol), 4-(morpholin-4-yl)phenol (231 mg, 1.29 mmol) and (2-chlorophenyl)acetic acid (224 mg, 1.31 mmol) were converted without purification of intermediates to 2-(2-chlorophenyl)-N-{4-[4-(morpholin-4-yl)phenoxy]-3-sulfamoylphenyl}acetamide and were purified at the end by preparative HPLC (Waters XBrigde C18 5μ 100×30 mm, acetonitrile/water+0.1% formic acid) (33 mg, 0.0657 mmol, 5% yield over 4 steps, 98% purity).

LC-MS (Method A): Rt=1.07 min

MS (ESIpos): m/z=502 (M+H)⁺

¹H-NMR (400 MHz, METHANOL-d₄) δ [ppm]: 3.09-3.16 (m, 4H), 3.81-3.85 (m, 4H), 3.87 (s, 2H), 6.82 (d, 1H), 7.00-7.08 (m, 4H), 7.25-7.32 (m, 2H), 7.37-7.43 (m, 2H), 7.69 (dd, 1H), 8.14 (d, 1H).

### Example 045

### 2-(2-Chlorophenyl)-N-[4-(5-cyano-2-methylphenoxy)-3-sulfamoylphenyl]acetamide

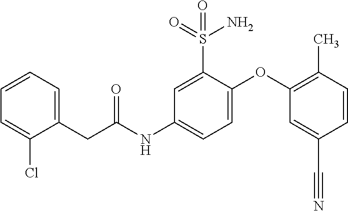 [SMILES: Cc1ccc(C#N)cc1Oc1ccc(NC(=O)Cc2ccccc2Cl)cc1S(N)(=O)=O]

According to general procedures GP1.1, GP2.2, GP3.2 and GP4, 2-chloro-N-(2,4-dimethoxybenzyl)-5-nitrobenzenesulfonamide (500 mg, 1.29 mmol), 3-hydroxy-4-methylbenzonitrile (172 mg, 1.29 mmol) and (2-chlorophenyl)acetic acid (242 mg, 1.42 mmol) were converted without purification of intermediates to 2-(2-chlorophenyl)-N-[4-(5-cyano-2-methylphenoxy)-3-sulfamoylphenyl]acetamide and were purified at the end by preparative HPLC (Waters XBrigde C18 5μ 100×30 mm, acetonitrile/water+0.1% formic acid) (53 mg, 0.116 mmol, 9% yield over 4 steps, 95% purity).

LC-MS (Method A): Rt=1.13 min

MS (ESIpos): m/z=456 (M+H)⁺

¹H-NMR (400 MHz, DMSO-d₆) δ [ppm]: 2.31 (s, 3H), 3.85 (s, 2H), 6.82 (d, 1H), 7.27 (d, 1H), 7.28-7.35 (m, 2H), 7.40-7.48 (m, 4H), 7.54 (d, 1H), 7.60 (dd, 1H), 7.74 (dd, 1H), 8.23 (d, 1H), 10.50 (s, 1H).

### Example 046

### 2-(2-Chlorophenyl)-N-[4-(3-cyano-2-methylphenoxy)-3-sulfamoylphenyl]acetamide

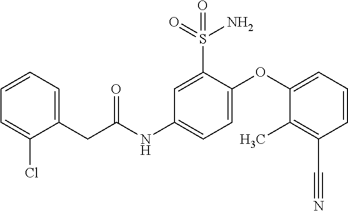 [SMILES: Cc1c(C#N)cccc1Oc1ccc(NC(=O)Cc2ccccc2Cl)cc1S(N)(=O)=O]

According to general procedures GP1.1, GP2.2, GP3.2 and GP4, 2-chloro-N-(2,4-dimethoxybenzyl)-5-nitrobenzenesulfonamide (500 mg, 1.29 mmol), 3-hydroxy-2-methylbenzonitrile (172 mg, 1.29 mmol) and (2-chlorophenyl)acetic acid (242 mg, 1.42 mmol) were converted without purification of intermediates to 2-(2-chlorophenyl)-N-[4-(3-cyano-2-methylphenoxy)-3-sulfamoylphenyl]acetamide and were purified at the end by preparative HPLC (Waters XBrigde C18 5μ 100×30 mm, acetonitrile/water+0.1% formic acid) (29 mg, 0.0636 mmol, 5% yield over 4 steps, 95% purity).

LC-MS (Method A): Rt=1.13 min

MS (ESIpos): m/z=456 (M+H)⁺

¹H-NMR (400 MHz, DMSO-d₆) δ [ppm]: 2.42 (s, 3H), 3.84 (s, 2H), 6.81 (d, 1H), 7.18 (dd, 1H), 7.28-7.35 (m, 2H), 7.39-7.48 (m, 5H), 7.64 (dd, 1H), 7.73 (dd, 1H), 8.22 (d, 1H), 10.48 (s, 1H).

### Example 047

### 2-(2-Chlorophenyl)-N-[4-(3-cyano-4-fluorophenoxy)-3-sulfamoylphenyl]acetamide

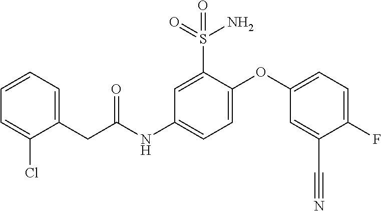 [SMILES: N#Cc1cc(Oc2ccc(NC(=O)Cc3ccccc3Cl)cc2S(N)(=O)=O)ccc1F]

According to general procedures GP1.1, GP2.2, GP3.2 and GP4, 2-chloro-N-(2,4-dimethoxybenzyl)-5-nitrobenzenesulfonamide (500 mg, 1.29 mmol), 2-fluoro-5-hydroxybenzonitrile (177 mg, 1.29 mmol) and (2-chlorophenyl)acetic acid (251 mg, 1.47 mmol) were converted without purification of intermediates to 2-(2-chlorophenyl)-N-[4-(3-cyano-4-fluorophenoxy)-3-sulfamoylphenyl]acetamide and were purified at the end by preparative HPLC (Waters XBrigde C18 5μ 100×30 mm, acetonitrile/water+0.1% formic acid) (46 mg, 0.100 mmol, 8% yield over 4 steps, 95% purity).

LC-MS (Method A): Rt=1.12 min

MS (ESIpos): m/z=460 (M+H)⁺

¹H-NMR (400 MHz, DMSO-d₆) δ [ppm]: 3.86 (s, 2H), 7.12 (d, 1H), 7.30-7.35 (m, 2H), 7.39-7.48 (m, 5H), 7.55 (t, 1H), 7.60 (dd, 1H), 7.82 (dd, 1H), 8.22 (d, 1H), 10.55 (s, 1H).

### Example 048

### N-{4-[(5-Chloro-2-cyanopyridin-3-yl)oxy]-3-sulfamoylphenyl}-2-(2-chlorophenyl)acetamide

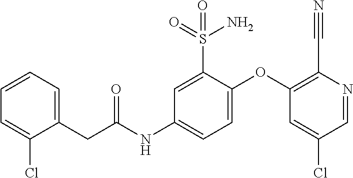 [SMILES: N#Cc1ncc(Cl)cc1Oc1ccc(NC(=O)Cc2ccccc2Cl)cc1S(N)(=O)=O]

According to general procedures GP1.1, GP2.2, GP3.2 and GP4, 2-chloro-N-(2,4-dimethoxybenzyl)-5-nitrobenzenesulfonamide (500 mg, 1.29 mmol), 5-chloro-3-hydroxypyridine-2-carbonitrile (200 mg, 1.29 mmol) and (2-chlorophenyl)acetic acid (242 mg, 1.42 mmol) were converted without purification of intermediates to N-{4-[(5-chloro-2-cyanopyridin-3-yl)oxy]-3-sulfamoylphenyl}-2-(2-chlorophenyl)acetamide and were purified at the end by preparative HPLC (Waters XBrigde C18 5μ 100×30 mm, acetonitrile/water+0.1% formic acid) (23 mg, 0.0482 mmol, 4% yield over 4 steps, 90% purity).

LC-MS (Method A): Rt=1.12 min

MS (ESIpos): m/z=477 (M+H)⁺

¹H-NMR (400 MHz, DMSO-d₆) δ [ppm]: 3.81 (s, 2H), 6.92 (d, 1H), 7.28-7.35 (m, 2H), 7.39-7.48 (m, 2H), 7.64 (dd, 1H), 7.69 (d, 1H), 8.19 (d, 1H), 8.39 (d, 1H), 10.30 (s, 1H), 10.92 (s, 1H), 11.40 (s, 1H).

### Example 049

### 2-(2-Chlorophenyl)-N-{4-[3-(piperidin-1-yl)phenoxy]-3-sulfamoylphenyl}acetamide

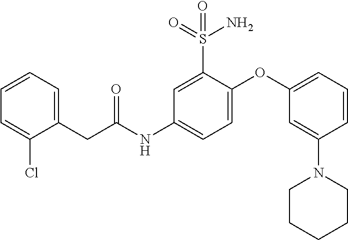 [SMILES: NS(=O)(=O)c1cc(NC(=O)Cc2ccccc2Cl)ccc1Oc1cccc(N2CCCCC2)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), 3-(piperidin-1-yl)phenol (229 mg, 1.29 mmol) and (2-chlorophenyl)acetic acid (242 mg, 1.42 mmol) were converted without purification of intermediates to 2-(2-chlorophenyl)-N-{4-[3-(piperidin-1-yl)phenoxy]-3-sulfamoylphenyl}acetamide and were purified at the end by preparative HPLC (Waters XBrigde C18 5μ 100×30 mm, acetonitrile/water+0.1% formic acid) (64 mg, 0.128 mmol, 10% yield over 4 steps, 98% purity).

LC-MS (Method A): Rt=1.15 min

MS (ESIpos): m/z=500 (M+H)⁺

¹H-NMR (400 MHz, DMSO-d₆) δ [ppm]: 1.49-1.65 (m, 6H), 3.09-3.18 (m, 4H), 3.84 (s, 2H), 6.39 (dd, 1H), 6.66 (t, 1H), 6.73 (dd, 1H), 6.93 (d, 1H), 7.18 (t, 1H), 7.25-7.35 (s, 4H), 7.41-7.48 (m, 2H), 7.72 (dd, 1H), 8.17 (d, 1H), 10.44 (s, 1H).

### Example 050

### 2-(2-Chlorophenyl)-N-{4-[3-(2-oxopyrrolidin-1-yl)phenoxy]-3-sulfamoylphenyl}acetamide

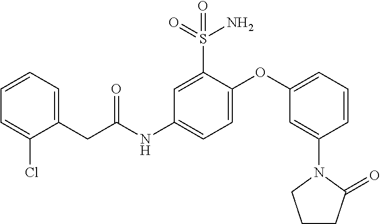 [SMILES: NS(=O)(=O)c1cc(NC(=O)Cc2ccccc2Cl)ccc1Oc1cccc(N2CCCC2=O)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), 1-(3-hydroxyphenyl)pyrrolidin-2-one (229 mg, 1.29 mmol) and (2-chlorophenyl)acetic acid (242 mg, 1.42 mmol) were converted without purification of intermediates to 2-(2-chlorophenyl)-N-{4-[3-(2-oxopyrrolidin-1-yl)phenoxy]-3-sulfamoylphenyl}acetamide and were purified at the end by preparative HPLC (Waters XBrigde C18 5μ 100×30 mm, acetonitrile/water+0.1% formic acid) (15 mg, 0.0300 mmol, 2% yield over 4 steps, 99% purity).

LC-MS (Method A): Rt=1.06 min

MS (ESIpos): m/z=500 (M+H)⁺

¹H-NMR (400 MHz, DMSO-d₆) δ [ppm]: 2.00-2.11 (m, 2H), one signal overlapped by solvent peak, 3.82 (t, 2H), 3.85 (s, 2H), 6.75-6.82 (m, 1H), 6.96 (d, 1H), 7.28-7.40 (m, 6H), 7.42-7.48 (m, 2H), 7.57-7.58 (m, 1H), 7.76 (dd, 1H), 8.20 (d, 1H), 10.49 (s, 1H).

### Example 051

### 2-(2-Chlorophenyl)-N-{4-[3-(2-oxo-1,3-oxazolidin-3-yl)phenoxy]-3-sulfamoylphenyl}acetamide

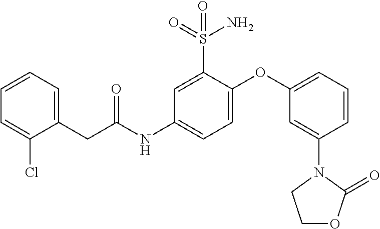 [SMILES: NS(=O)(=O)c1cc(NC(=O)Cc2ccccc2Cl)ccc1Oc1cccc(N2CCOC2=O)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), 3-(3-hydroxyphenyl)-1,3-oxazolidin-2-one (231 mg, 1.29 mmol) and (2-chlorophenyl)acetic acid (242 mg, 1.42 mmol) were converted without purification of intermediates to 2-(2-chlorophenyl)-N-{4-[3-(2-oxo-1,3-oxazolidin-3-yl)phenoxy]-3-sulfamoylphenyl}acetamide and were purified at the end by preparative HPLC (Waters XBrigde C18 5μ 100×30 mm, acetonitrile/water+0.1% formic acid) (42 mg, 0.0837 mmol, 6% yield over 4 steps, 95% purity).

LC-MS (Method A): Rt=1.04 min

MS (ESIpos): m/z=502 (M+H)⁺

¹H-NMR (400 MHz, DMSO-d₆) δ [ppm]: 3.85 (s, 2H), 4.05 (dd, 2H), 4.44 (dd, 2H), 6.77 (ddd, 1H), 6.98 (d, 1H), 7.26-7.48 (m, 9H), 7.77 (dd, 1H), 8.20 (d, 1H), 10.49 (s, 1H).

### Example 052

### 2-(2-Chlorophenyl)-N-{4-[3-(morpholin-4-ylcarbonyl)phenoxy]-3-sulfamoylphenyl}acetamide

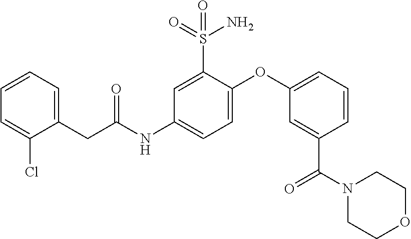 [SMILES: NS(=O)(=O)c1cc(NC(=O)Cc2ccccc2Cl)ccc1Oc1cccc(C(=O)N2CCOCC2)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), (3-hydroxyphenyl)(morpholin-4-yl)methanone (268 mg, 1.29 mmol) and (2-chlorophenyl)acetic acid (242 mg, 1.42 mmol) were converted without purification of intermediates to 2-(2-chlorophenyl)-N-{4-[3-(morpholin-4-ylcarbonyl)phenoxy]-3-sulfamoylphenyl}acetamide and were purified at the end by preparative HPLC (Waters XBrigde C18 5μ 100×30 mm, acetonitrile/water+0.1% formic acid) (65 mg, 0.123 mmol, 10% yield over 4 steps, 90% purity).

LC-MS (Method A): Rt=0.99 min

MS (ESIpos): m/z=530 (M+H)⁺

¹H-NMR (400 MHz, DMSO-d₆) δ [ppm]: 3.44-3.70 (m, 8H), 3.85 (s, 2H), 7.02 (dd, 1H), 7.06 (s, 1H), 7.13 (ddd, 1H), 7.18 (dt, 1H), 7.28-7.35 (m, 2H), 7.39 (s, 2H), 7.42-7.47 (m, 3H), 7.78 (dd, 1H), 8.22 (d, 1H), 10.52 (s, 1H).

### Example 053

### 2-(2-Chlorophenyl)-N-{4-[(4-methyltetrahydro-2H-pyran-4-yl)methoxy]-3-sulfamoylphenyl}acetamide

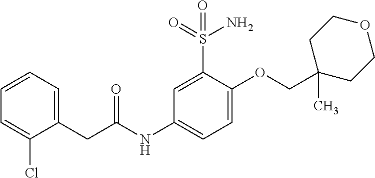 [SMILES: CC1(COc2ccc(NC(=O)Cc3ccccc3Cl)cc2S(N)(=O)=O)CCOCC1]

According to general procedure GP1.1, 2-chloro-N-(2,4-dimethoxybenzyl)-5-nitrobenzenesulfonamide (500 mg, 1.29 mmol) and (4-methyltetrahydro-2H-pyran-4-yl)methanol (168 mg, 1.29 mmol) were converted to N-(2,4-dimethoxybenzyl)-2-[(4-methyltetrahydro-2H-pyran-4-yl)methoxy]-5-nitrobenzenesulfonamide) which was purified via column chromatography on silica gel (91 mg, 0.189 mmol, 15% yield).

Then, according to general procedures GP2.2, GP3.2 and GP4, this crude N-(2,4-dimethoxybenzyl)-2-[(4-methyltetrahydro-2H-pyran-4-yl)methoxy]-5-nitrobenzenesulfonamide) and (2-chlorophenyl)acetic acid (21.7 mg, 0.13 mmol) were converted without purification of further intermediates to 2-(2-chlorophenyl)-N-{4-[(4-methyltetrahydro-2H-pyran-4-yl)methoxy]-3-sulfamoylphenyl}acetamide and were purified at the end purified by preparative HPLC (Chromatorex C-18 10 μm, 125×30 mm, acetonitrile/water+0.1% formic acid) (4 mg, 0.00883 mmol, 1% yield over 4 steps, 95% purity).

LC-MS (Method A): Rt=1.06 min

MS (ESIpos): m/z=453 (M+H)⁺

¹H-NMR (400 MHz, DMSO-d₆) δ [ppm]: 1.14 (s, 3H), 1.34-1.43 (m, 2H), 1.58-1.69 (m, 2H), 3.50-3.60 (m, 2H), 3.62-3.70 (m, 2H), 3.82 (s, 2H), 3.88 (s, 2H), 6.83 (s, 2H), 7.21 (d, 1H), 7.26-7.36 (m, 2H), 7.38-7.49 (m, 2H), 7.77 (dd, 1H), 8.04 (d, 1H), 10.33 (s, 1H).

### Example 054

### 2-(2-chlorophenyl)-N-{4-[(4-fluorotetrahydro-2H-pyran-4-yl)methoxy]-3-sulfamoylphenyl}acetamide

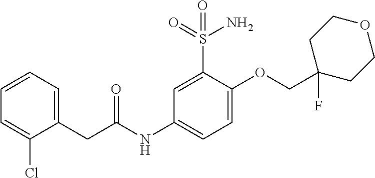 [SMILES: NS(=O)(=O)c1cc(NC(=O)Cc2ccccc2Cl)ccc1OCC1(F)CCOCC1]

According to general procedure GP1.1, 2-chloro-N-(2,4-dimethoxybenzyl)-5-nitrobenzenesulfonamide (500 mg, 1.29 mmol) and (4-fluorotetrahydro-2H-pyran-4-yl)methanol (173 mg, 1.29 mmol) were converted to N-(2,4-dimethoxybenzyl)-2-[(4-fluorotetrahydro-2H-pyran-4-yl)methoxy]-5-nitrobenzenesulfonamide which was purified via column chromatography on silica gel (173 mg, 0.357 mmol, 28% yield).

Then, according to general procedures GP2.2, GP3.2 and GP4, this crude N-(2,4-dimethoxybenzyl)-2-[(4-fluorotetrahydro-2H-pyran-4-yl)methoxy]-5-nitrobenzenesulfonamide and (2-chlorophenyl)acetic acid (59.5 mg, 0.35 mmol) were converted without purification of further intermediates to 2-(2-chlorophenyl)-N-{4-[(4-fluorotetrahydro-2H-pyran-4-yl)methoxy]-3-sulfamoylphenyl}acetamide and were purified at the end purified by preparative HPLC (Chromatorex C-18 10 μm, 125×30 mm, acetonitrile/water+0.1% formic acid) (28 mg, 0.0613 mmol, 5% yield over 4 steps, 99% purity).

LC-MS (Method A): Rt=1.16 min

MS (ESIpos): m/z=457 (M+H)⁺

¹H-NMR (400 MHz, DMSO-d₆) δ [ppm]: 1.81-1.96 (m, 4H), 3.55-3.65 (m, 2H), 3.74-3.82 (m, 2H), 3.83 (s, 2H), 4.26 (d, 2H), 6.81 (s, 2H), 7.23 (d, 1H), 7.28-7.36 (m, 2H), 7.40-7.48 (m, 2H), 7.79 (dd, 1H), 8.05 (d, 1H), 10.37 (s, 1H).

### Example 055

### 2-(2-Chlorophenyl)-N-{4-[(4-cyanotetrahydro-2H-pyran-4-yl)methoxy]-3-sulfamoylphenyl}acetamide

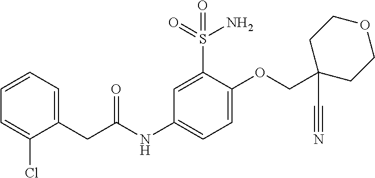 [SMILES: N#CC1(COc2ccc(NC(=O)Cc3ccccc3Cl)cc2S(N)(=O)=O)CCOCC1]

According to general procedures GP1.1, GP2.2, GP3.2 and GP4, 2-chloro-N-(2,4-dimethoxybenzyl)-5-nitrobenzenesulfonamide (500 mg, 1.29 mmol), 4-(hydroxymethyl)tetrahydro-2H-pyran-4-carbonitrile (182 mg, 1.29 mmol) and (2-chlorophenyl)acetic acid (183 mg, 1.08 mmol) were converted without purification of intermediates to 2-(2-chlorophenyl)-N-{4-[(4-cyanotetrahydro-2H-pyran-4-yl)methoxy]-3-sulfamoylphenyl}acetamide and were purified at the end by preparative HPLC (Waters XBrigde C18 5μ 100×30 mm, acetonitrile/water+0.2% aqueous ammonia (32%)) followed by another preparative HPLC (Chromatorex C-18 10 μm, 125×30 mm, acetonitrile/water+0.1% aqueous ammonia (32%)) (56 mg, 0.121 mmol, 9% yield over 4 steps, 95% purity).

LC-MS (Method B): Rt=0.93 min

MS (ESIpos): m/z=464 (M+H)⁺

¹H-NMR (400 MHz, DMSO-d₆) δ [ppm]: 1.72-1.85 (m, 2H), 1.97-2.05 (m, 2H), 3.53 (t, 2H), 3.83 (s, 2H), 3.91-4.00 (m, 2H), 4.29 (s, 2H), 6.82 (s, 2H), 7.23 (d, 1H), 7.28-7.36 (m, 2H), 7.40-7.47 (m, 2H), 7.82 (dd, 1H), 8.07 (d, 1H), 10.39 (s, 1H).

### Example 056

### 2-(2-Chlorophenyl)-N-(3-sulfamoyl-4-{[2-(trifluoromethyl)pyrimidin-5-yl]oxy}phenyl)acetamide

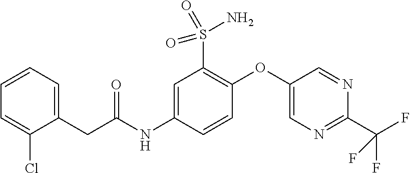 [SMILES: NS(=O)(=O)c1cc(NC(=O)Cc2ccccc2Cl)ccc1Oc1cnc(C(F)(F)F)nc1]

According to general procedure GP4, purified 2-(2-chlorophenyl)-N-(3-[(2,4-dimethoxybenzyl)sulfamoyl]-4-{[2-(trifluoromethyl)pyrimidin-5-yl]oxy}phenyl)acetamide (31.9 mg, 0.05 mmol) was converted to 2-(2-chlorophenyl)-N-(3-sulfamoyl-4-{[2-(trifluoromethyl)pyrimidin-5-yl]oxy}phenyl)acetamide and was purified by column chromatography on a Biotage Isolera system (silica gel, gradient dichloromethane/ethyl acetate) (21 mg, 0.0431 mmol, 86% yield, 95% purity).

LC-MS (Method A): Rt=1.15 min

MS (ESIpos): m/z=487 (M+H)⁺

¹H-NMR (400 MHz, DMSO-d₆) δ [ppm]: 3.81 (s, 2H), 6.91 (d, 1H), 7.28-7.36 (m, 2H), 7.40-7.47 (m, 2H), 7.63 (dd, 1H), 8.17 (d, 1H), 8.71 (s, 2H), 10.28 (s, 1H), 10.78-11.35 (m, 2H).

### Example 057

### 2-(2-Chlorophenyl)-N-{4-[(2-isopropylpyrimidin-5-yl)oxy]-3-sulfamoylphenyl}acetamide

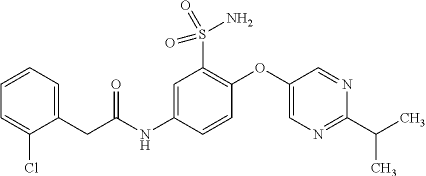 [SMILES: CC(C)c1ncc(Oc2ccc(NC(=O)Cc3ccccc3Cl)cc2S(N)(=O)=O)cn1]

According to general procedure GP4, purified 2-(2-chlorophenyl)-N-{3-[(2,4-dimethoxybenzyl)sulfamoyl]-4-[(2-isopropylpyrimidin-5-yl)oxy]phenyl}acetamide (100 mg, 0.164 mmol) was converted to 2-(2-chlorophenyl)-N-{4-[(2-isopropylpyrimidin-5-yl)oxy]-3-sulfamoylphenyl}acetamide and was purified by column chromatography on a Biotage Isolera system (silica gel, gradient dichloromethane/ethyl acetate) (13 mg, 0.0282 mmol, 17% yield, 99% purity).

LC-MS (Method A): Rt=1.10 min

MS (ESIpos): m/z=461 (M+H)⁺

¹H-NMR (400 MHz, DMSO-d₆) δ [ppm]: 1.28 (d, 6H), 3.15 (sep, 1H), 3.86 (s, 2H), 7.20 (d, 1H), 7.29-7.36 (m, 2H), 7.41-7.52 (m, 4H), 7.81 (dd, 1H), 8.24 (d, 1H), 8.47 (s, 2H), 10.56 (s, 1H).

### Example 058

### 2-(2-Chlorophenyl)-N-{4-[(2-cyclopropyl-4-methylpyrimidin-5-yl)oxy]-3-sulfamoylphenyl}acetamide

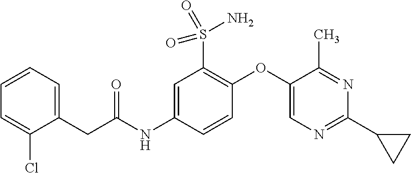 [SMILES: Cc1nc(C2CC2)ncc1Oc1ccc(NC(=O)Cc2ccccc2Cl)cc1S(N)(=O)=O]

According to general procedure GP4, purified 2-(2-chlorophenyl)-N-{4-[(2-cyclopropyl-4-methylpyrimidin-5-yl)oxy]-3-[(2,4-dimethoxybenzyl)sulfamoyl]phenyl}acetamide (50 mg, 0.0802 mmol) was converted to 2-(2-chlorophenyl)-N-{4-[(2-cyclopropyl-4-methylpyrimidin-5-yl)oxy]-3-sulfamoylphenyl}acetamide and was purified by column chromatography on a Biotage Isolera system (silica gel, gradient dichloromethane/ethyl acetate) (25 mg, 0.0529 mmol, 66% yield, 99% purity).

LC-MS (Method A): Rt=1.11 min

MS (ESIpos): m/z=473 (M+H)⁺

¹H-NMR (400 MHz, DMSO-d₆) δ [ppm]: 0.94-0.99 (m, 2H), 1.00-1.05 (m, 2H), 2.15-2.23 (m, 1H), 2.33 (s, 3H), 3.84 (s, 2H), 6.85 (d, 1H), 7.28-7.35 (m, 2H), 7.40-7.48 (m, 4H), 7.73 (dd, 1H), 8.18 (s, 1H), 8.21 (d, 1H), 10.47 (s, 1H).

### Example 059

### N-[4-(3-Bromophenoxy)-3-sulfamoylphenyl]-2-(2-chlorophenyl)acetamide

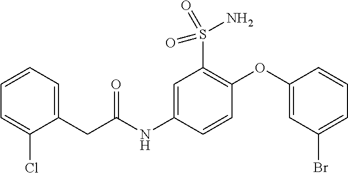 [SMILES: NS(=O)(=O)c1cc(NC(=O)Cc2ccccc2Cl)ccc1Oc1cccc(Br)c1]

According to general procedure GP4, N-{4-(3-bromophenoxy)-3-[(2,4-dimethoxybenzyl)sulfamoyl]phenyl}-2-(2-chlorophenyl)acetamide (400 mg, 40% purity) was converted to N-[4-(3-bromophenoxy)-3-sulfamoylphenyl]-2-(2-chlorophenyl)acetamide and was purified by two column chromatographies on a Biotage Isolera system (silica gel, gradient dichloromethane/ethyl acetate and dichloromethane/dichloromethane-methanol) (30 mg, 97% purity).

LC-MS (Method A): Rt=1.23 min

MS (ESIpos): m/z=497 (M+H)⁺

¹H-NMR (400 MHz, DMSO-d₆) δ [ppm]: 3.86 (s, 2H), 7.00-7.06 (m, 1H), 7.10 (d, 1H), 7.22-7.24 (m, 1H), 7.29-7.37 (m, 4H), 7.39 (s, 2H), 7.42-7.49 (m, 2H), 7.80 (dd, 1H), 8.22 (d, 1H), 10.53 (s, 1H).

### Example 060

### N-[4-(4-Bromophenoxy)-3-sulfamoylphenyl]-2-(2-chlorophenyl)acetamide

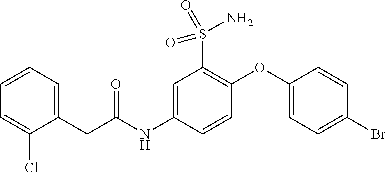 [SMILES: NS(=O)(=O)c1cc(NC(=O)Cc2ccccc2Cl)ccc1Oc1ccc(Br)cc1]

According to general procedure GP4, N-{4-(4-bromophenoxy)-3-[(2,4-dimethoxybenzyl)sulfamoyl]phenyl}-2-(2-chlorophenyl)acetamide (400 mg, 40% purity) was converted to N-[4-(4-bromophenoxy)-3-sulfamoylphenyl]-2-(2-chlorophenyl)acetamide and was purified by two column chromatographies on a Biotage Isolera system (silica gel, gradient dichloromethane/ethyl acetate and dichloromethane to dichloromethane-methanol) (20 mg, 97% purity).

LC-MS (Method A): Rt=1.26 min

MS (ESIpos): m/z=497 (M+H)⁺

¹H-NMR (400 MHz, DMSO-d₆) δ [ppm]: 3.85 (s, 2H), 6.97-7.02 (m, 2H), 7.04 (d, 1H), 7.29-7.36 (m, 2H), 7.39 (s, 2H), 7.41-7.48 (m, 2H), 7.54-7.58 (m, 2H), 7.78 (dd, 1H), 8.21 (d, 1H), 10.53 (s, 1H).

### Example 061

### 2-(2-Chlorophenyl)-N-{4-[3-(2-methyl-1,3-thiazol-4-yl)phenoxy]-3-sulfamoylphenyl}acetamide

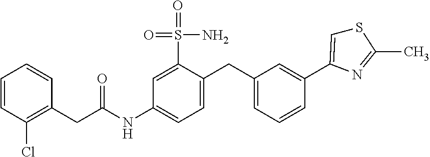 [SMILES: Cc1nc(-c2cccc(Cc3ccc(NC(=O)Cc4ccccc4Cl)cc3S(N)(=O)=O)c2)cs1]

According to general procedures GP1.1, GP2.2, GP3.2 and GP4, 2-chloro-N-(2,4-dimethoxybenzyl)-5-nitrobenzenesulfonamide (500 mg, 1.29 mmol), 3-(2-methyl-1,3-thiazol-4-yl)phenol (247 mg, 1.29 mmol) and (2-chlorophenyl)acetic acid (215 mg, 1.26 mmol) were converted without purification of intermediates to 2-(2-chlorophenyl)-N-{4-[3-(2-methyl-1,3-thiazol-4-yl)phenoxy]-3-sulfamoylphenyl}acetamide and were purified at the end by preparative HPLC (Waters XBrigde C18 5μ 100×30 mm, acetonitrile/water+0.1% formic acid) (43 mg, 0.0837 mmol, 6% yield over 4 steps, 99% purity).

LC-MS (Method A): Rt=1.21 min

MS (ESIpos): m/z=514 (M+H)⁺

¹H-NMR (400 MHz, DMSO-d₆) δ [ppm]: 2.70 (s, 3H), 3.85 (s, 2H), 6.98-7.03 (m, 2H), 7.30-7.35 (m, 2H), 7.40 (s, 2H), 7.42-7.48 (m, 3H), 7.65 (t, 1H), 7.71-7.80 (m, 2H), 7.98 (s, 1H), 8.22 (d, 1H), 10.51 (s, 1H).

### Example 062

### 2-(2-Chlorophenyl)-N-{4-[4-(5-oxopyrrolidin-2-yl)phenoxy]-3-sulfamoylphenyl}acetamide

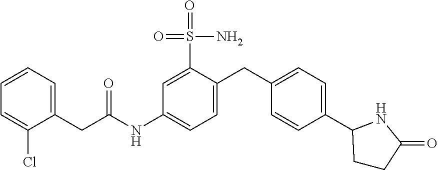 [SMILES: NS(=O)(=O)c1cc(NC(=O)Cc2ccccc2Cl)ccc1Cc1ccc(C2CCC(=O)N2)cc1]

According to general procedures GP1.1, GP2.2, GP3.2 and GP4, 2-chloro-N-(2,4-dimethoxybenzyl)-5-nitrobenzenesulfonamide (500 mg, 1.29 mmol), 5-(4-hydroxyphenyl)pyrrolidin-2-one (229 mg, 1.29 mmol) and (2-chlorophenyl)acetic acid (144 mg, 0.845 mmol) were converted without purification of intermediates to 2-(2-chlorophenyl)-N-{4-[3-(2-methyl-1,3-thiazol-4-yl)phenoxy]-3-sulfamoylphenyl}acetamide and were purified at the end by preparative HPLC (Waters XBrigde C18 5μ 100×30 mm, acetonitrile/water+0.1% formic acid) (25.2 mg, 0.0504 mmol, 4% yield over 4 steps, 98% purity).

LC-MS (Method A): Rt=0.96 min

MS (ESIpos): m/z=500 (M+H)⁺

¹H-NMR (400 MHz, DMSO-d₆) δ [ppm]: 1.69-1.81 (m, 1H), 2.24 (t, 2H), one signal overlapped by solvent peak, 3.85 (s, 2H), 4.67 (t, 1H), 6.95 (d, 1H), 7.04 (d, 2H), 7.23-7.38 (m, 6H), 7.41-7.48 (m, 2H), 7.76 (dd, 1H), 8.10 (s, 1H), 8.19 (d, 1H), 10.50 (s, 1H).

### Example 063

### 2-(2-chlorophenyl)-N-{4-[4-(2-oxo-1,3-oxazolidin-3-yl)phenoxy]-3-sulfamoylphenyl}acetamide

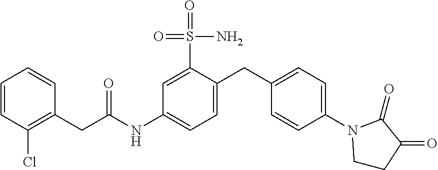 [SMILES: NS(=O)(=O)c1cc(NC(=O)Cc2ccccc2Cl)ccc1Cc1ccc(N2CCC(=O)C2=O)cc1]

According to general procedures GP1.1, GP2.2, GP3.2 and GP4, 2-chloro-N-(2,4-dimethoxybenzyl)-5-nitrobenzenesulfonamide (500 mg, 1.29 mmol), 3-(4-hydroxyphenyl)-1,3-oxazolidin-2-one (232 mg, 1.29 mmol) and (2-chlorophenyl)acetic acid (220 mg, 1.29 mmol) were converted without purification of intermediates to 2-(2-chlorophenyl)-N-{4-[4-(2-oxo-1,3-oxazolidin-3-yl)phenoxy]-3-sulfamoylphenyl}acetamide and were purified at the end by preparative HPLC (Waters XBrigde C18 5μ 100×30 mm, acetonitrile/water+0.1% formic acid) (79 mg, 0.157 mmol, 12% yield over 4 steps, 98% purity).

LC-MS (Method A): Rt=1.01 min

MS (ESIpos): m/z=502 (M+H)⁺

¹H-NMR (400 MHz, DMSO-d₆) δ [ppm]: 3.84 (s, 2H), 4.06 (dd, 2H), 4.44 (dd, 2H), 6.92 (d, 1H), 7.07-7.14 (m, 2H), 7.28-7.40 (m, 4H), 7.41-7.48 (m, 2H), 7.55-7.62 (m, 2H), 7.75 (dd, 1H), 8.19 (d, 1H), 10.48 (s, 1H).

### Example 064

### 2-(2-Chlorophenyl)-N-{3-sulfamoyl-4-[4-(1,3-thiazol-2-yl)phenoxy]phenyl}acetamide

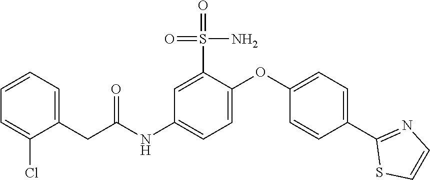 [SMILES: NS(=O)(=O)c1cc(NC(=O)Cc2ccccc2Cl)ccc1Oc1ccc(-c2nccs2)cc1]

According to general procedures GP1.1, GP2.2, GP3.2 and GP4, 2-chloro-N-(2,4-dimethoxybenzyl)-5-nitrobenzenesulfonamide (500 mg, 1.29 mmol), 4-(1,3-thiazol-2-yl)phenol (229 mg, 1.29 mmol) and (2-chlorophenyl)acetic acid (248 mg, 1.46 mmol) were converted without purification of intermediates to 2-(2-chlorophenyl)-N-{3-sulfamoyl-4-[4-(1,3-thiazol-2-yl)phenoxy]phenyl}acetamide and were purified at the end by preparative HPLC (Waters XBrigde C18 5μ 100×30 mm, acetonitrile/water+0.1% formic acid) (69 mg, 0.138 mmol, 11% yield over 4 steps, 98% purity).

LC-MS (Method A): Rt=1.15 min

MS (ESIpos): m/z=500 (M+H)⁺

¹H-NMR (400 MHz, DMSO-d₆) δ [ppm]: 3.86 (s, 2H), 7.10-7.17 (m, 3H), 7.30-7.36 (m, 2H), 7.40-7.48 (m, 4H), 7.76 (d, 1H), 7.81 (dd, 1H), 7.91 (d, 1H), 7.95-7.99 (m, 2H), 8.24 (d, 1H), 10.55 (s, 1H).

### Example 065

### N-[4-(2-Chlorophenoxy)-3-sulfamoylphenyl]-2-(2-chlorophenyl)acetamide

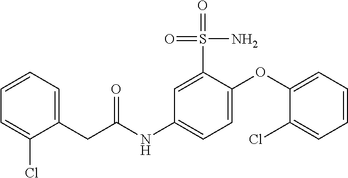 [SMILES: NS(=O)(=O)c1cc(NC(=O)Cc2ccccc2Cl)ccc1Oc1ccccc1Cl]

According to general procedures GP1.1, GP2.2, GP3.2 and GP4, 2-chloro-N-(2,4-dimethoxybenzyl)-5-nitrobenzenesulfonamide (600 mg, 1.55 mmol), 2-chlorophenol (199 mg, 1.55 mmol) and (2-chlorophenyl)acetic acid (146 mg, 0.858 mmol) were converted without purification of intermediates to N-[4-(2-chlorophenoxy)-3-sulfamoylphenyl]-2-(2-chlorophenyl)acetamide and were purified at the end by preparative HPLC (Waters XBrigde C18 5μ 100×30 mm, acetonitrile/water+0.1% formic acid) (12.5 mg, 0.0277 mmol, 2% yield over 4 steps, 99% purity).

LC-MS (Method A): Rt=1.17 min

MS (ESIpos): m/z=451 (M+H)⁺

¹H-NMR (400 MHz, DMSO-d₆) δ [ppm]: 3.84 (s, 2H), 6.73 (d, 1H), 7.12 (dd, 1H), 7.26 (td, 1H), 7.29-7.35 (m, 2H), 7.35-7.40 (m, 3H), 7.41-7.47 (m, 2H), 7.62 (dd, 1H), 7.72 (dd, 1H), 8.21 (d, 1H), 10.48 (s, 1H).

### Example 066

### N-[4-(4-Chlorophenoxy)-3-sulfamoylphenyl]-2-(2-chlorophenyl)acetamide

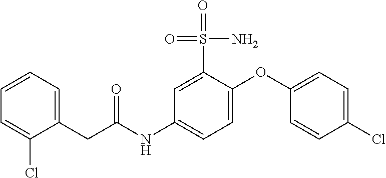 [SMILES: NS(=O)(=O)c1cc(NC(=O)Cc2ccccc2Cl)ccc1Oc1ccc(Cl)cc1]

According to general procedures GP1.1, GP2.2, GP3.2 and GP4, 2-chloro-N-(2,4-dimethoxybenzyl)-5-nitrobenzenesulfonamide (300 mg, 0.776 mmol), 4-chlorophenol (100 mg, 0.776 mmol) and (2-chlorophenyl)acetic acid (121 mg, 0.708 mmol) were converted without purification of intermediates to N-[4-(4-chlorophenoxy)-3-sulfamoylphenyl]-2-(2-chlorophenyl)acetamide and were purified at the end by preparative HPLC (Waters XBrigde C18 5μ 100×30 mm, acetonitrile/water+0.1% formic acid) (9 mg, 0.0199 mmol, 3% yield over 4 steps, 99% purity).

LC-MS (Method A): Rt=1.21 min

MS (ESIpos): m/z=451 (M+H)⁺

¹H-NMR (400 MHz, DMSO-d₆) δ [ppm]: 3.85 (s, 2H), 7.00-7.09 (m, 3H), 7.28-7.35 (m, 2H), 7.39 (s, 2H), 7.41-7.48 (m, 4H), 7.78 (dd, 1H), 8.20 (d, 1H), 10.52 (s, 1H).

### Example 067

### 2-(2-chlorophenyl)-N-{4-[3-(piperidin-1-ylcarbonyl)phenoxy]-3-sulfamoylphenyl}acetamide

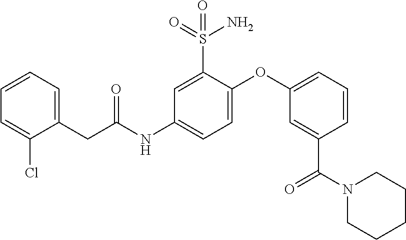 [SMILES: NS(=O)(=O)c1cc(NC(=O)Cc2ccccc2Cl)ccc1Oc1cccc(C(=O)N2CCCCC2)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), (3-hydroxyphenyl)(piperidin-1-yl)methanone (265 mg, 1.29 mmol) and (2-chlorophenyl)acetic acid (242 mg, 1.42 mmol) were converted without purification of intermediates to 2-(2-chlorophenyl)-N-{4-[3-(piperidin-1-ylcarbonyl)phenoxy]-3-sulfamoylphenyl}acetamide and were purified at the end by preparative HPLC (Waters XBrigde C18 5μ 100×30 mm, acetonitrile/water+0.1% formic acid) (38 mg, 0.0720 mmol, 6% yield over 4 steps, 98% purity).

LC-MS (Method A): Rt=1.12 min

MS (ESIpos): m/z=528 (M+H)⁺

¹H-NMR (400 MHz, DMSO-d₆) δ [ppm]: 1.35-1.65 (m, 6H), 3.18-3.32 (m, 2H), 3.46-3.63 (m, 2H), 3.85 (s, 2H), 6.95-6.99 (m, 1H), 7.05 (d, 1H), 7.10-7.14 (m, 2H), 7.28-7.35 (m, 2H), 7.37-7.49 (m, 5H), 7.78 (dd, 1H), 8.22 (d, 1H), 10.52 (s, 1H).

### Example 068

### 2-(2-Chlorophenyl)-N-{3-sulfamoyl-4-[4-(tetrahydrofuran-3-yl)phenoxy]phenyl}acetamide

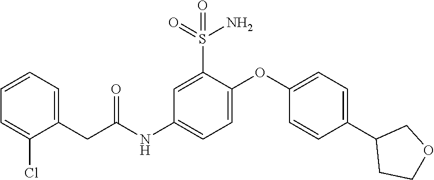 [SMILES: NS(=O)(=O)c1cc(NC(=O)Cc2ccccc2Cl)ccc1Oc1ccc(C2CCOC2)cc1]

According to general procedures GP1.1, GP2.2, GP3.2 and GP4, 2-chloro-N-(2,4-dimethoxybenzyl)-5-nitrobenzenesulfonamide (500 mg, 1.29 mmol), 4-(tetrahydrofuran-3-yl)phenol (212 mg, 1.29 mmol) and (2-chlorophenyl)acetic acid (242 mg, 1.42 mmol) were converted without purification of intermediates to 2-(2-chlorophenyl)-N-{3-sulfamoyl-4-[4-(tetrahydrofuran-3-yl)phenoxy]phenyl}acetamide and were purified at the end by preparative HPLC (Waters XBrigde C18 5μ 100×30 mm, acetonitrile/water+0.1% formic acid) (45 mg, 0.0924 mmol, 7% yield over 4 steps, 98% purity).

LC-MS (Method A): Rt=1.12 min

MS (ESIpos): m/z=487 (M+H)⁺

¹H-NMR (400 MHz, DMSO-d₆) δ [ppm]: 1.86-1.96 (m, 1H), 2.25-2.35 (m, 1H), 3.38 (quin, 1H), 3.53 (t, 1H), 3.80 (q, 1H), 3.84 (s, 2H), 3.95 (td, 1H), 4.03 (t, 1H), 6.94 (d, 1H), 6.98-7.03 (m, 2H), 7.28-7.36 (m, 6H), 7.41-7.47 (m, 2H), 7.74 (dd, 1H), 8.19 (d, 1H), 10.48 (s, 1H).

### Example 069

### 2-(2-chlorophenyl)-N-[4-(3-cyano-5-fluorophenoxy)-3-sulfamoylphenyl]acetamide

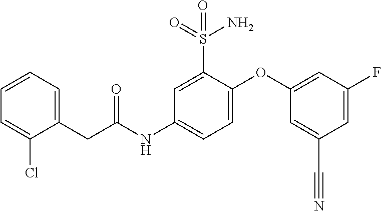 [SMILES: N#Cc1cc(F)cc(Oc2ccc(NC(=O)Cc3ccccc3Cl)cc2S(N)(=O)=O)c1]

According to general procedures GP1.1, GP2.2, GP3.2 and GP4, 2-chloro-N-(2,4-dimethoxybenzyl)-5-nitrobenzenesulfonamide (500 mg, 1.29 mmol) 3-fluoro-5-hydroxybenzonitrile (177 mg, 1.29 mmol) and (2-chlorophenyl)acetic acid (242 mg, 1.42 mmol) were converted without purification of intermediates to 2-(2-chlorophenyl)-N-[4-(3-cyano-5-fluorophenoxy)-3-sulfamoylphenyl]acetamide and were purified at the end by preparative HPLC (Waters XBrigde C18 5μ 100×30 mm, acetonitrile/water+0.1% formic acid) (50 mg, 0.109 mmol, 8% yield over 4 steps, 95% purity).

LC-MS (Method A): Rt=1.11 min

MS (ESIpos): m/z=460 (M+H)⁺

¹H-NMR (400 MHz, DMSO-d₆) δ [ppm]: 3.80 (s, 2H), 6.90 (d, 1H), 7.20 (dt, 1H), 7.25-7.35 (m, 3H), 7.40-7.48 (m, 3H), 7.63 (dd, 1H), 8.10 (d, 1H), 10.27 (s, 1H), 10.82 (s, 2H).

### Example 070

### N-[4-(2-Methoxyphenoxy)-3-sulfamoylphenyl]-2-phenylacetamide

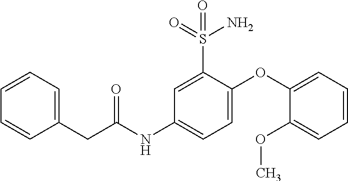 [SMILES: COc1ccccc1Oc1ccc(NC(=O)Cc2ccccc2)cc1S(N)(=O)=O]

According to general procedures GP1.1, GP2.2, GP3.2 and GP4, 2-chloro-N-(2,4-dimethoxybenzyl)-5-nitrobenzenesulfonamide (300 mg, 0.776 mmol), 2-methoxyphenol (96 mg, 0.776 mmol) and phenylacetic acid (116 mg, 0.854 mmol) were converted without purification of intermediates to N-[4-(2-methoxyphenoxy)-3-sulfamoylphenyl]-2-phenylacetamide and were purified at the end by preparative HPLC (Waters XBrigde C18 5μ 100×30 mm, acetonitrile/water+0.1% formic acid) (26 mg, 0.0630 mmol, 8% yield over 4 steps, 99% purity).

LC-MS (Method A): Rt=1.07 min

MS (ESIpos): m/z=413 (M+H)⁺

¹H-NMR (400 MHz, DMSO-d₆) δ [ppm]: 3.62 (s, 2H), 3.75 (s, 3H), 6.63 (d, 1H), 7.00 (td, 1H), 7.10 (dd, 1H), 7.17-7.29 (m, 5H), 7.32-7.34 (m, 4H), 7.66 (dd, 1H), 8.13 (d, 1H), 10.35 (s, 1H).

### Example 071

### N-[4-(2-Methoxyphenoxy)-3-sulfamoylphenyl]-2-[4-(trifluoromethyl)phenyl]acetamide

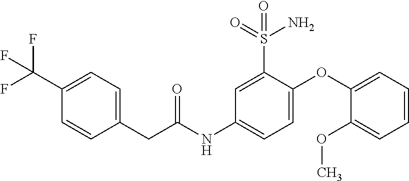 [SMILES: COc1ccccc1Oc1ccc(NC(=O)Cc2ccc(C(F)(F)F)cc2)cc1S(N)(=O)=O]

According to general procedures GP1.1, GP2.2, GP3.2 and GP4, 2-chloro-N-(2,4-dimethoxybenzyl)-5-nitrobenzenesulfonamide (300 mg, 0.776 mmol), 2-methoxyphenol (96 mg, 0.776 mmol) and [4-(trifluoromethyl)phenyl]acetic acid (174 mg, 0.854 mmol) were converted without purification of intermediates to N-[4-(2-methoxyphenoxy)-3-sulfamoylphenyl]-2-[4-(trifluoromethyl)phenyl]acetamide and were purified at the by preparative HPLC (Waters XBrigde C18 5μ 100×30 mm, acetonitrile/water+0.1% formic acid) (46 mg, 0.0957 mmol, 12% yield over 4 steps, 99% purity).

LC-MS (Method A): Rt=1.21 min

MS (ESIpos): m/z=481 (M+H)⁺

¹H-NMR (400 MHz, DMSO-d₆) δ [ppm]: 3.75 (s, 5H), 6.64 (d, 1H), 7.00 (td, 1H), 7.11 (dd, 1H), 7.17-7.28 (m, 4H), 7.55 (d, 2H), 7.65 (dd, 1H), 7.70 (d, 2H), 8.13 (d, 1H), 10.42 (s, 1H).

### Example 072

### N-{3-Sulfamoyl-4-[2-(trifluoromethoxy)phenoxy]phenyl}-2-[4-(trifluoromethyl)phenyl]acetamide

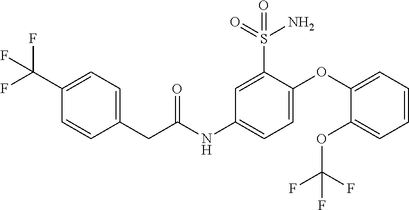 [SMILES: NS(=O)(=O)c1cc(NC(=O)Cc2ccc(C(F)(F)F)cc2)ccc1Oc1ccccc1OC(F)(F)F]

According to general procedures GP1.1, GP2.2, GP3.2 and GP4, 2-chloro-N-(2,4-dimethoxybenzyl)-5-nitrobenzenesulfonamide (300 mg, 0.776 mmol), 2-(trifluoromethoxy)phenol (138 mg, 0.776 mmol) and [4-(trifluoromethyl)phenyl]acetic acid (176 mg, 0.854 mmol) were converted without purification of intermediates to N-{3-sulfamoyl-4-[2-(trifluoromethoxy)phenoxy]phenyl}-2-[4-(trifluoromethyl)phenyl]acetamide and were purified at the end by preparative HPLC (Waters XBrigde C18 5μ 100×30 mm, acetonitrile/water+0.1% formic acid) (6 mg, 0.0112 mmol, 1% yield over 4 steps, 99% purity).

LC-MS (Method A): Rt=1.26 min

MS (ESIpos): m/z=535 (M+H)⁺

¹H-NMR (400 MHz, DMSO-d₆) δ [ppm]: 3.78 (s, 2H), 6.84 (d, 1H), 7.04 (dd, 1H), 7.29 (td, 1H), 7.33-7.43 (m, 3H), 7.51-7.58 (m, 3H), 7.71 (d, 2H), 7.76 (dd, 1H), 8.21 (d, 1H), 10.54 (s, 1H).

### Example 073

### N-[4-(2-Chlorophenoxy)-3-sulfamoylphenyl]-2-[4-(trifluoromethyl)phenyl]acetamide

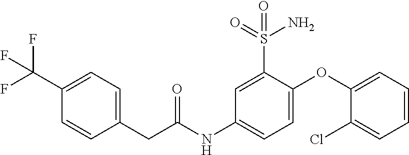 [SMILES: NS(=O)(=O)c1cc(NC(=O)Cc2ccc(C(F)(F)F)cc2)ccc1Oc1ccccc1Cl]

According to general procedures GP1.1, GP2.2, GP3.2 and GP4, 2-chloro-N-(2,4-dimethoxybenzyl)-5-nitrobenzenesulfonamide (300 mg, 0.775 mmol), 2-chlorophenol (100 mg, 0.775 mmol) and [4-(trifluoromethyl)phenyl]acetic acid (175 mg, 0.857 mmol) were converted without purification of intermediates to N-[4-(2-chlorophenoxy)-3-sulfamoylphenyl]-2-[4-(trifluoromethyl)phenyl]acetamide and were purified at the end by preparative HPLC (Waters XBrigde C18 5μ 100×30 mm, acetonitrile/water+0.1% formic acid) (13 mg, 0.0268 mmol, 3% yield over 4 steps, 98% purity).

LC-MS (Method A): Rt=1.20 min

MS (ESIneg): m/z=483 (M−H)⁺

¹H-NMR (400 MHz, DMSO-d₆) δ [ppm]: 3.77 (s, 2H), 6.72 (d, 1H), 7.11 (dd, 1H), 7.26 (td, 1H), 7.33-7.42 (m, 3H), 7.56 (d, 2H), 7.62 (dd, 1H), 7.67-7.75 (m, 3H), 8.19 (d, 1H), 10.50 (s, 1H).

### Example 074

### 2-Phenyl-N-{3-sulfamoyl-4-[2-(trifluoromethoxy)phenoxy]phenyl}acetamide

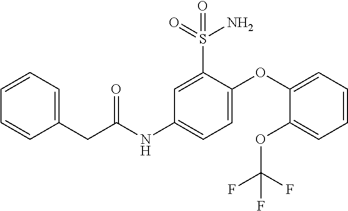 [SMILES: NS(=O)(=O)c1cc(NC(=O)Cc2ccccc2)ccc1Oc1ccccc1OC(F)(F)F]

According to general procedures GP1.1, GP2.2, GP3.2 and GP4, 2-chloro-N-(2,4-dimethoxybenzyl)-5-nitrobenzenesulfonamide (300 mg, 0.776 mmol), 2-(trifluoromethoxy)phenol (138 mg, 0.776 mmol) and phenylacetic acid (117 mg, 0.854 mmol) were converted without purification of intermediates to 2-phenyl-N-{3-sulfamoyl-4-[2-(trifluoromethoxy)phenoxy]phenyl}acetamide and were purified at the end by preparative HPLC (Waters XBrigde C18 5μ 100×30 mm, acetonitrile/water+0.1% formic acid) (6 mg, 0.0129 mmol, 2% yield over 4 steps, 98% purity).

LC-MS (Method A): Rt=1.14 min

MS (ESIneg): m/z=465 (M−H)⁺

¹H-NMR (400 MHz, DMSO-d₆) δ [ppm]: 3.64 (s, 2H), 6.84 (d, 1H), 7.03 (dd, 1H), 7.22-7.42 (m, 9H), 7.50-7.55 (m, 1H), 7.77 (dd, 1H), 8.21 (d, 1H), 10.46 (s, 1H).

### Example 075

### 2-(2-Chlorophenyl)-N-{4-[(2-oxo-1,2-dihydropyridin-3-yl)oxy]-3-sulfamoylphenyl}acetamide

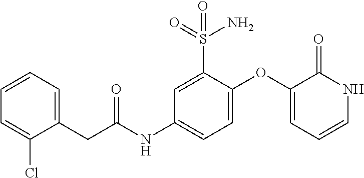 [SMILES: NS(=O)(=O)c1cc(NC(=O)Cc2ccccc2Cl)ccc1Oc1ccc[nH]c1=O]

According to general procedures GP1.1, GP2.2, GP3.2 and GP4, 2-chloro-N-(2,4-dimethoxybenzyl)-5-nitrobenzenesulfonamide (500 mg, 1.29 mmol), 3-hydroxypyridin-2(1H)-one (143 mg, 1.29 mmol) and (2-chlorophenyl)acetic acid (243 mg, 1.42 mmol) were converted without purification of intermediates to 2-(2-chlorophenyl)-N-{4-[(2-oxo-1,2-dihydropyridin-3-yl)oxy]-3-sulfamoylphenyl}acetamide and were purified at the end by preparative HPLC (Waters XBrigde C18 5μ 100×30 mm, acetonitrile/water+0.1% formic acid) (3.5 mg, 0.0807 mmol, 1% yield over 4 steps, 85% purity).

LC-MS (Method A): Rt=0.88 min

MS (ESIpos): m/z=434 (M+H)⁺

### Example 076

### N-[4-(2-Chlorophenoxy)-3-sulfamoylphenyl]-2-phenylacetamide

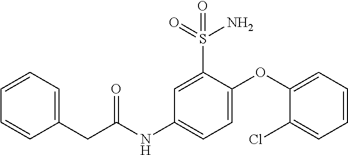 [SMILES: NS(=O)(=O)c1cc(NC(=O)Cc2ccccc2)ccc1Oc1ccccc1Cl]

According to general procedures GP1.1, GP2.2, GP3.2 and GP4, 2-chloro-N-(2,4-dimethoxybenzyl)-5-nitrobenzenesulfonamide (500 mg, 1.29 mmol), 2-chlorophenol (166 mg, 1.29 mmol) and phenylacetic acid (193 mg, 1.42 mmol) were converted without purification of intermediates to N-[4-(2-chlorophenoxy)-3-sulfamoylphenyl]-2-phenylacetamide and were purified at the end by preparative HPLC (Waters XBrigde C18 5μ 100×30 mm, acetonitrile/water+0.1% formic acid) (28 mg, 0.0672 mmol, 5% yield over 4 steps, 98% purity).

LC-MS (Method A): Rt=1.11 min

MS (ESIpos): m/z=417 (M+H)⁺

¹H-NMR (300 MHz, DMSO-d₆) δ [ppm]: 3.62 (s, 2H), 6.71 (d, 1H), 7.09 (dd, 1H), 7.20-7.41 (m, 9H), 7.59 (dd, 1H), 7.71 (dd, 1H), 8.18 (d, 1H), 10.41 (s, 1H).

### Example 077

### N-[4-(4-Chlorophenoxy)-3-sulfamoylphenyl]-2-phenylacetamide

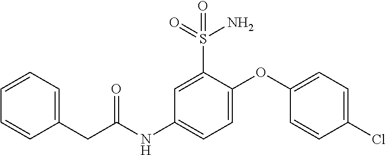 [SMILES: NS(=O)(=O)c1cc(NC(=O)Cc2ccccc2)ccc1Oc1ccc(Cl)cc1]

According to general procedures GP1.1, GP2.2, GP3.2 and GP4, 2-chloro-N-(2,4-dimethoxybenzyl)-5-nitrobenzenesulfonamide (500 mg, 1.29 mmol), 4-chlorophenol (166 mg, 1.29 mmol) and phenylacetic acid (193 mg, 1.42 mmol) were converted without purification of intermediates to N-[4-(4-chlorophenoxy)-3-sulfamoylphenyl]-2-phenylacetamide and were purified at the end by preparative HPLC (Waters XBrigde C18 5μ 100×30 mm, acetonitrile/water+0.1% formic acid) (25 mg, 0.0600 mmol, 5% yield over 4 steps, 98% purity).

LC-MS (Method A): Rt=1.16 min

MS (ESIpos): m/z=417 (M+H)⁺

¹H-NMR (300 MHz, METHANOL-d₄) δ [ppm]: [ppm]=3.68 (s, 2H), 6.93 (d, 1H), 7.04-7.11 (m, 2H), 7.22-7.40 (m, 7H), 7.77 (dd, 1H), 8.19 (d, 1H).

### Example 078

### N-{4-[(5-Chloropyridin-3-yl)oxy]-3-sulfamoylphenyl}-2-phenylacetamide

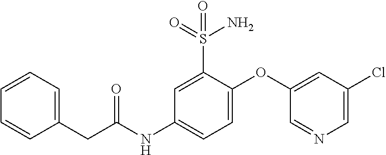 [SMILES: NS(=O)(=O)c1cc(NC(=O)Cc2ccccc2)ccc1Oc1cncc(Cl)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), 5-chloropyridin-3-ol (167 mg, 1.29 mmol) and phenylacetic acid (206 mg, 1.52 mmol) were converted without purification of intermediates to N-{4-[(5-chloropyridin-3-yl)oxy]-3-sulfamoylphenyl}-2-phenylacetamide and were purified at the end by preparative HPLC (Waters XBrigde C18 5μ 100×30 mm, acetonitrile/water+0.1% formic acid) followed by another preparative HPLC (Waters XBrigde C18 5μ 100×30 mm, acetonitrile/water+0.2% aqueous ammonia (32%)) (20.7 mg, 0.0495 mmol, 4% yield over 4 steps, 98% purity).

LC-MS (Method B): Rt=0.90 min

MS (ESIpos): m/z=418 (M+H)⁺

¹H-NMR (400 MHz, DMSO-d₆) δ [ppm]: 3.65 (s, 2H), 7.18-7-27 (m, 2H), 7.29-7.36 (m, 4H), 7.45 (s, 2H), 7.50 (t, 1H), 7.84 (dd, 1H), 8.21 (d, 1H), 8.30 (d, 1H), 8.38 (d, 1H), 10.49 (s, 1H).

### Example 079

### N-{4-[(5-Chloropyridin-3-yl)oxy]-3-sulfamoylphenyl}-2-phenylacetamide

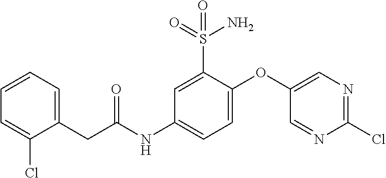 [SMILES: NS(=O)(=O)c1cc(NC(=O)Cc2ccccc2Cl)ccc1Oc1cnc(Cl)nc1]

According to general procedures GP1.1, GP2.2, GP3.2 and GP4, 2-fluoro-N-(2,4-dimethoxybenzyl)-5-nitrobenzenesulfonamide (185 mg, 0.50 mmol), 2-chloropyrimidin-5-ol (65.3 mg, 0.50 mmol) and (2-chlorophenyl)acetic acid (51.2 mg, 0.30 mmol) were converted with purification of intermediates on a Biotage Isolera system to N-{4-[(5-chloropyridin-3-yl)oxy]-3-sulfamoylphenyl}-2-phenylacetamide and were purified at the end by preparative HPLC (Chromatorex C-18 10 μm, 125×30 mm, acetonitrile/water+0.1% formic acid) (10 mg, 0.0221 mmol, 4% yield over 4 steps, 95% purity).

LC-MS (Method A): Rt=1.03 min

MS (ESIpos): m/z=453 (M+H)⁺

¹H-NMR (400 MHz, DMSO-d₆) δ [ppm]: 3.80 (s, 2H), 6.89 (d, 1H), 7.27-7.36 (m, 2H), 7.39-7.48 (m, 2H), 7.61 (dd, 1H), 8.07 (d, 1H), 8.43 (s, 2H), 10.25 (s, 1H), 10.80 (s, 2H).

### Example 080

### 2-(2-Chlorophenyl)-N-{4-[(5-fluoropyridin-3-yl)oxy]-3-sulfamoylphenyl}acetamide

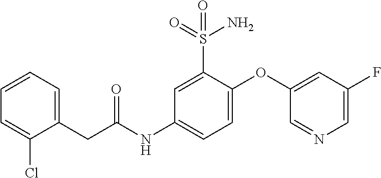 [SMILES: NS(=O)(=O)c1cc(NC(=O)Cc2ccccc2Cl)ccc1Oc1cncc(F)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), 5-fluoropyridin-3-ol (146 mg, 1.29 mmol) and (2-chlorophenyl)acetic acid (242 mg, 1.42 mmol) were converted without purification of intermediates to 2-(2-chlorophenyl)-N-{4-[(5-fluoropyridin-3-yl)oxy]-3-sulfamoylphenyl}acetamide and were purified at the end by preparative HPLC (Waters XBrigde C18 5μ 100×30 mm, acetonitrile/water+0.1% formic acid), followed by another preparative HPLC (Waters XBrigde C18 5μ 100×30 mm, acetonitrile/water+0.2% aqueous ammonia (32%)) (13.7 mg, 0.0314 mmol, 2% yield over 4 steps, 99% purity).

LC-MS (Method B): Rt=0.93 min

MS (ESIpos): m/z=436 (M+H)⁺

¹H-NMR (400 MHz, DMSO-d₆) δ [ppm]: 3.87 (s, 2H), 7.23 (d, 1H), 7.29-7.52 (m, 7H), 7.84 (dd, 1H), 8.24 (d, 1H), 8.25-8.27 (m, 1H), 8.38 (d, 1H), 10.59 (s, 1H).

### Example 081

### 2-(2-Chlorophenyl)-N-{4-[(6-chloropyridin-3-yl)oxy]-3-sulfamoylphenyl}acetamide

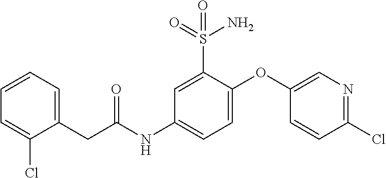 [SMILES: NS(=O)(=O)c1cc(NC(=O)Cc2ccccc2Cl)ccc1Oc1ccc(Cl)nc1]

According to general procedures GP1.1, GP2.2, GP3.2 and GP4, 2-chloro-N-(2,4-dimethoxybenzyl)-5-nitrobenzenesulfonamide (500 mg, 1.29 mmol), 6-chloropyridin-3-ol (167 mg, 1.29 mmol) and (2-chlorophenyl)acetic acid (242 mg, 1.42 mmol) were converted without purification of intermediates to 2-(2-chlorophenyl)-N-{4-[(6-chloropyridin-3-yl)oxy]-3-sulfamoylphenyl}acetamide and were purified at the end by preparative HPLC (Waters XBrigde C18 5μ 100×30 mm, acetonitrile/water+0.1% formic acid), followed by another preparative HPLC (Waters XBrigde C18 5μ 100×30 mm, acetonitrile/water+0.2% aqueous ammonia (32%)) (10.0 mg, 0.0221 mmol, 2% yield over 4 steps, 99% purity).

LC-MS (Method B): Rt=1.02 min

MS (ESIpos): m/z=452 (M+H)⁺

¹H-NMR (400 MHz, DMSO-d₆) δ [ppm]: 3.86 (s, 2H), 7.18 (d, 1H), 7.28-7.36 (m, 2H), 7.40-7.55 (m, 6H), 7.80-7.86 (m, 1H), 8.20 (d, 1H), 8.22-8.26 (m, 1H), 10.57 (s, 1H).

### Example 082

### N-[2-chloro-4-(3-chlorophenoxy)-5-sulfamoylphenyl]-2-(2-chlorophenyl)acetamide

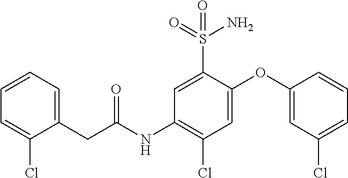 [SMILES: NS(=O)(=O)c1cc(NC(=O)Cc2ccccc2Cl)c(Cl)cc1Oc1cccc(Cl)c1]

According to general procedures GP1.1, GP2.2, GP3.2 and GP4, 2,4-dichloro-N-(2,4-dimethoxybenzyl)-5-nitrobenzenesulfonamide (157 mg, 0.373 mmol), 3-chlorophenol (43.1 mg, 0.335 mmol) and (2-chlorophenyl)acetic acid (69.8 mg, 0.410 mmol) were converted without purification of intermediates to N-[2-chloro-4-(3-chlorophenoxy)-5-sulfamoylphenyl]-2-(2-chlorophenyl)acetamide and were purified at the end by preparative HPLC (Chromatorex C-18 10 μm, 125×30 mm, acetonitrile/water+0.1% formic acid) (6.5 mg, 0.0134 mmol, 4% yield over 4 steps, 90% purity).

LC-MS (Method A): Rt=1.27 min

MS (ESIpos): m/z=485 (M+H)⁺

¹H-NMR (400 MHz, DMSO-d₆) δ [ppm]: 3.91 (s, 2H), 7.07 (ddd, 1H), 7.16 (s, 1H), 7.23 (t, 1H), 7.27-7.45 (m, 5H), 7.48 (t, 1H), 7.61 (s, 2H), 8.75 (s, 1H), 10.08 (s, 1H).

### Example 083

### N-[2-chloro-4-(3-chlorophenoxy)-5-sulfamoylphenyl]-2-(2-chloro-3-fluorophenyl)acetamide

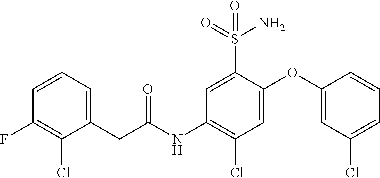 [SMILES: NS(=O)(=O)c1cc(NC(=O)Cc2cccc(F)c2Cl)c(Cl)cc1Oc1cccc(Cl)c1]

According to general procedures GP1.1, GP2.2, GP3.2 and GP4, 2,4-dichloro-N-(2,4-dimethoxybenzyl)-5-nitrobenzenesulfonamide (157 mg, 0.373 mmol), 3-chlorophenol (43.1 mg, 0.335 mmol) and (2-chloro-3-fluorophenyl)acetic acid (77.3 mg, 0.410 mmol) were converted without purification of intermediates to N-[2-chloro-4-(3-chlorophenoxy)-5-sulfamoylphenyl]-2-(2-chloro-3-fluorophenyl)acetamide and were purified at the end by preparative HPLC (Chromatorex C-18 10 μm, 125×30 mm, acetonitrile/water+0.1% formic acid) (3.8 mg, 0.00754 mmol, 2% yield over 4 steps, 85% purity).

LC-MS (Method A): Rt=1.27 min

MS (ESIneg): m/z=501 (M−H)⁺

¹H-NMR (400 MHz, DMSO-d₆) δ [ppm]: 3.97 (s, 2H), 7.08 (dd, 1H), 7.15-7.38 (m, 6H), 7.48 (t, 1H), 7.62 (s, 2H), 8.74 (s, 1H), 10.15 (s, 1H).

### Example 084

### N-[4-(3-Chlorophenoxy)-3-sulfamoylphenyl]-2-(3-fluorophenyl)acetamide

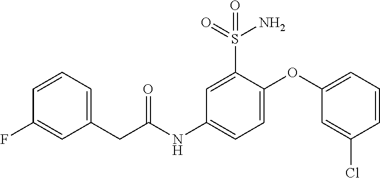 [SMILES: NS(=O)(=O)c1cc(NC(=O)Cc2cccc(F)c2)ccc1Oc1cccc(Cl)c1]

According to general procedures GP3.3 and GP4, purified 5-amino-2-(3-chlorophenoxy)-N-(2,4-dimethoxybenzyl)benzenesulfonamide (112 mg, 0.25 mmol) and (3-fluorophenyl)acetic acid (77.0 mg, 0.50 mmol) were converted to N-[4-(3-chlorophenoxy)-3-sulfamoylphenyl]-2-(3-fluorophenyl)acetamide and were purified by HPLC (11.2 mg, 0.258 mmol, 10% yield, 99% purity).

LC-MS (Method A): Rt=1.13 min

MS (ESIpos): m/z=435 (M+H)⁺

¹H-NMR (400 MHz, DMSO-d₆) δ [ppm]: 3.70 (s, 2H), 6.98 (ddd, 1H), 7.07-7.13 (m, 3H), 7.16-7.22 (m, 3H), 7.35-7.42 (m, 4H), 7.81 (dd, 1H), 8.20 (d, 1H), 10.50 (s, 1H).

### Example 085

### N-[4-(3-Chlorophenoxy)-3-sulfamoylphenyl]-2-(4-fluorophenyl)acetamide

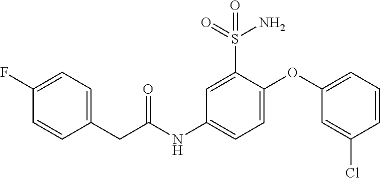 [SMILES: NS(=O)(=O)c1cc(NC(=O)Cc2ccc(F)cc2)ccc1Oc1cccc(Cl)c1]

According to general procedures GP3.3 and GP4, purified 5-amino-2-(3-chlorophenoxy)-N-(2,4-dimethoxybenzyl)benzenesulfonamide (112 mg, 0.25 mmol) and (4-fluorophenyl)acetic acid (77.0 mg, 0.50 mmol) were converted to N-[4-(3-chlorophenoxy)-3-sulfamoylphenyl]-2-(4-fluorophenyl)acetamide and were purified by HPLC (12.9 mg, 0.297 mmol, 12% yield, 99% purity).

LC-MS (Method A): Rt=1.12 min

MS (ESIpos): m/z=435 (M+H)⁺

¹H-NMR (400 MHz, DMSO-d₆) δ [ppm]: 3.65 (s, 2H), 6.97 (ddd, 1H), 7.07-7.11 (m, 2H), 7.18 (d, 3H), 7.34-7.43 (m, 5H), 7.81 (dd, 1H), 8.20 (d, 1H), 10.48 (s, 1H).

### Example 086

### N-[4-(3-Chlorophenoxy)-3-sulfamoylphenyl]-2-[2-(trifluoromethyl)phenyl]acetamide

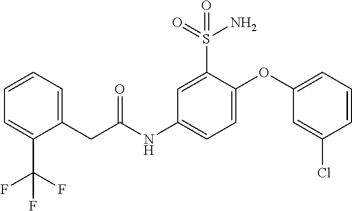 [SMILES: NS(=O)(=O)c1cc(NC(=O)Cc2ccccc2C(F)(F)F)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and [2-(trifluoromethyl)phenyl]acetic acid (35.0 mg, 0.172 mmol) were converted to N-[4-(3-chlorophenoxy)-3-sulfamoylphenyl]-2-[2-(trifluoromethyl)phenyl]acetamide and were purified by preparative HPLC (Chromatorex C-18 10 μm, 125×30 mm, acetonitrile/water+0.1% formic acid) (62 mg, 0.128 mmol, 82% yield, 97% purity).

LC-MS (Method A): Rt=1.28 min

MS (ESIpos): m/z=485 (M+H)⁺

¹H-NMR (400 MHz, DICHLOROMETHANE-d₂) δ [ppm]: 3.93 (s, 2H), 5.16 (s, 2H), 6.96 (d, 1H), 7.00 (ddd, 1H), 7.10 (t, 1H), 7.18-7.24 (m, 1H), 7.31-7.38 (m, 2H), 7.47-7.57 (m, 2H), 7.60-7.67 (m, 1H), 7.75 (d, 1H), 7.82 (dd, 1H), 7.92 (d, 1H).

### Example 087

### N-[4-(3-Chlorophenoxy)-3-sulfamoylphenyl]-2-(2-isopropylphenyl)acetamide

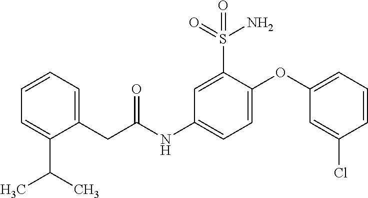 [SMILES: CC(C)c1ccccc1CC(=O)Nc1ccc(Oc2cccc(Cl)c2)c(S(N)(=O)=O)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and (2-isopropylphenyl)acetic acid (30.6 mg, 0.172 mmol) were converted to N-[4-(3-chlorophenoxy)-3-sulfamoylphenyl]-2-(2-isopropylphenyl)acetamide and were by preparative HPLC (Waters XBrigde C18 5μ 100×30 mm, acetonitrile/water+0.1% formic acid) (35 mg, 0.0763 mmol, 49% yield, 99% purity).

LC-MS (Method A): Rt=1.30 min

MS (ESIpos): m/z=459 (M+H)⁺

¹H-NMR (500 MHz, DICHLOROMETHANE-d₂) δ [ppm]: 3.82 (s, 2H), 5.15 (s., 2H), 6.95 (d, 1H), 6.99 (dd, 1H), 7.08-7.12 (m, 1H), 7.18-7.30 (m, 4H), 7.31-7.45 (m, 3H), 7.78 (dd, 1H), 7.85 (d, 1H).

### Example 088

### N-[4-(3-Chlorophenoxy)-3-sulfamoylphenyl]-2-(2-ethoxyphenyl)acetamide

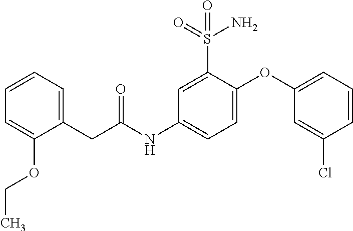 [SMILES: CCOc1ccccc1CC(=O)Nc1ccc(Oc2cccc(Cl)c2)c(S(N)(=O)=O)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and (2-ethoxyphenyl)acetic acid (30.9 mg, 0.172 mmol) were converted to N-[4-(3-chlorophenoxy)-3-sulfamoylphenyl]-2-(2-ethoxyphenyl)acetamide and were purified by preparative HPLC (Waters XBrigde C18 5μ 100×30 mm, acetonitrile/water+0.1% formic acid) (30 mg, 0.0651 mmol, 42% yield, 99% purity).

LC-MS (Method A): Rt=1.22 min

MS (ESIpos): m/z=461 (M+H)⁺

¹H-NMR (500 MHz, DICHLOROMETHANE-d₂) δ [ppm]: 1.49 (t, 3H), 3.72 (s, 2H), 4.19 (q, 2H), 5.14 (s, 2H), 6.95 (d, 1H), 6.97-7.02 (m, 3H), 7.09 (t, 1H), 7.20 (ddd, 1H), 7.29-7.37 (m, 3H), 7.81 (dd, 1H), 7.88 (d, 1H), 8.00 (s, 1H).

### Example 089

### N-[4-(3-Chlorophenoxy)-3-sulfamoylphenyl]-2-[2-(difluoromethyl)phenyl]acetamide

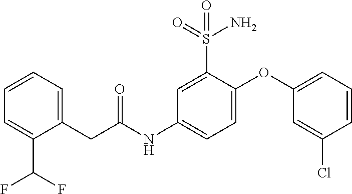 [SMILES: NS(=O)(=O)c1cc(NC(=O)Cc2ccccc2C(F)F)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and [2-(difluoromethyl)phenyl]acetic acid (31.9 mg, 0.172 mmol) were converted to N-[4-(3-chlorophenoxy)-3-sulfamoylphenyl]-2-[2-(difluoromethyl)phenyl]acetamide and were purified by preparative HPLC (Waters XBrigde C18 5μ 100×30 mm, acetonitrile/water+0.1% formic acid) (35 mg, 0.0750 mmol, 48% yield, 99% purity).

LC-MS (Method A): Rt=1.19 min

MS (ESIpos): m/z=467 (M+H)⁺

¹H-NMR (500 MHz, DICHLOROMETHANE-d₂) δ [ppm]: 3.91 (s, 2H), 5.16 (s, 2H), 6.89 (t, 1H), 6.95 (d, 1H), 6.98-7.01 (m, 1H), 7.10 (t, 1H), 7.18-7.23 (m, 1H), 7.35 (t, 1H), 7.42-7.50 (m, 3H), 7.52-7.58 (m, 1H), 7.60 (d, 1H), 7.81 (dd, 1H), 7.91 (d, 1H).

### Example 090

### N-[4-(3-Chlorophenoxy)-3-sulfamoylphenyl]-2-{2-[(trifluoromethyl)sulfanyl]phenyl}acetamide

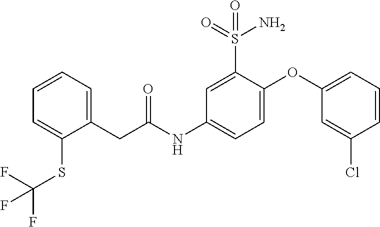 [SMILES: NS(=O)(=O)c1cc(NC(=O)Cc2ccccc2SC(F)(F)F)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and {2-[(trifluoromethyl)sulfanyl]phenyl}acetic acid (40.5 mg, 0.172 mmol) were converted to N-[4-(3-chlorophenoxy)-3-sulfamoylphenyl]-2-{2-[(trifluoromethyl)sulfanyl]phenyl}acetamide and were purified by preparative HPLC (Waters XBrigde C18 5μ 100×30 mm, acetonitrile/water+0.1% formic acid) (35 mg, 0.0677 mmol, 43% yield, 99% purity).

LC-MS (Method A): Rt=1.31 min

MS (ESIpos): m/z=517 (M+H)⁺

¹H-NMR (500 MHz, DICHLOROMETHANE-d₂) δ [ppm]: 4.06 (s, 2H), 5.17 (s, 2H), 6.97 (d, 1H), 6.99-7.03 (m, 1H), 7.11 (t, 1H), 7.19-7.23 (m, 1H), 7.35 (t, 1H), 7.45 (td, 1H), 7.49 (s, 1H), 7.52-7.60 (m, 2H), 7.79-7.86 (m, 2H), 7.95 (d, 1H).

### Example 091

### 2-(2-Bromophenyl)-N-[4-(3-chlorophenoxy)-3-sulfamoylphenyl]acetamide

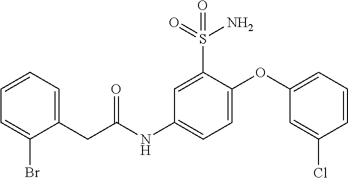 [SMILES: NS(=O)(=O)c1cc(NC(=O)Cc2ccccc2Br)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and (2-bromophenyl)acetic acid (36.9 mg, 0.172 mmol) were converted to 2-(2-bromophenyl)-N-[4-(3-chlorophenoxy)-3-sulfamoylphenyl]acetamide and were purified by preparative HPLC (Waters XBrigde C18 5μ 100×30 mm, acetonitrile/water+0.1% formic acid) (35 mg, 0.0706 mmol, 45% yield, 99% purity).

LC-MS (Method A): Rt=1.21 min

MS (ESIpos): m/z=497 (M+H)⁺

¹H-NMR (500 MHz, DICHLOROMETHANE-d₂) δ [ppm]: 3.89 (s, 2H), 5.16 (s, 2H), 6.96 (d, 1H), 6.98-7.02 (m, 1H), 7.11 (t, 1H), 7.19-7.23 (m, 1H), 7.25 (td, 1H), 7.35 (t, 1H), 7.39 (td, 1H), 7.42-7.46 (m, 2H), 7.66 (dd, 1H), 7.85 (dd, 1H), 7.92 (d, 1H).

### Example 092

### N-[4-(3-Chlorophenoxy)-3-sulfamoylphenyl]-2-(4-methylpyridin-3-yl)acetamide

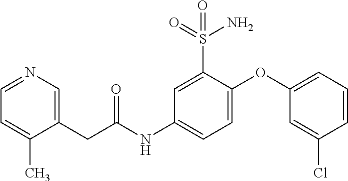 [SMILES: Cc1ccncc1CC(=O)Nc1ccc(Oc2cccc(Cl)c2)c(S(N)(=O)=O)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and (4-methylpyridin-3-yl)acetic acid (25.9 mg, 0.172 mmol) were converted to N-[4-(3-chlorophenoxy)-3-sulfamoylphenyl]-2-(4-methylpyridin-3-yl)acetamide and were purified by preparative HPLC (Waters XBrigde C18 5μ 100×30 mm, acetonitrile/water+0.1% formic acid) (10 mg, 0.0232 mmol, 15% yield, 98% purity).

LC-MS (Method A): Rt=0.78 min

MS (ESIpos): m/z=432 (M+H)⁺

¹H-NMR (400 MHz, DMSO-d₆) δ [ppm]: 2.43 (s, 3H), 3.64 (s, 2H), 6.96 (dd, 1H), 7.04-7.09 (m, 2H), 7.16-7.23 (m, 2H), 7.34-7.41 (m, 3H), 7.62 (dd, 1H), 7.79 (dd, 1H), 8.17 (d, 1H), 8.37 (d, 1H), 10.47 (s, 1H).

### Example 093

### N-[4-(3-Chlorophenoxy)-3-sulfamoylphenyl]-2-(2-chloropyridin-3-yl)acetamide

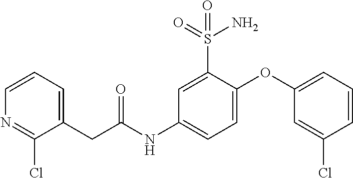 [SMILES: NS(=O)(=O)c1cc(NC(=O)Cc2cccnc2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and (2-chloropyridin-3-yl)acetic acid (29.4 mg, 0.172 mmol) were converted to N-[4-(3-chlorophenoxy)-3-sulfamoylphenyl]-2-(2-chloropyridin-3-yl)acetamide and were purified by preparative HPLC (Waters XBrigde C18 5μ 100×30 mm, acetonitrile/water+0.1% formic acid) (30 mg, 0.0663 mmol, 43% yield, 99% purity).

LC-MS (Method A): Rt=1.04 min

MS (ESIpos): m/z=452 (M+H)⁺

¹H-NMR (500 MHz, DICHLOROMETHANE-d₂) δ [ppm]: 3.85 (s, 2H), 5.23 (s, 2H), 6.97 (d, 1H), 7.01 (dd, 1H), 7.11 (t, 1H), 7.17-7.24 (m, 1H), 7.29-7.38 (m, 2H), 7.68-7.76 (m, 1H), 7.78 (dd, 1H), 7.85 (dd, 1H), 7.98 (d, 1H), 8.35 (d, 1H).

### Example 094

### N-[4-(3-Chlorophenoxy)-3-sulfamoylphenyl]-2-(2-chlorophenyl)-2,2-difluoroacetamide

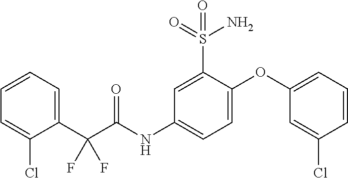 [SMILES: NS(=O)(=O)c1cc(NC(=O)C(F)(F)c2ccccc2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and (2-chlorophenyl)(difluoro)acetic acid (35.4 mg, 0.172 mmol) were converted to N-[4-(3-chlorophenoxy)-3-sulfamoylphenyl]-2-(2-chlorophenyl)-2,2-difluoroacetamide and were purified by preparative HPLC (Chromatorex C-18 10 μm, 125×30 mm, acetonitrile/water+0.1% formic acid) (21 mg, 0.0431 mmol, 28% yield, 98% purity).

LC-MS (Method A): Rt=1.27 min

MS (ESIneg): m/z=485 (M−H)⁺

¹H-NMR (500 MHz, DICHLOROMETHANE-d₂) δ [ppm]: 5.20 (s, 2H), 7.01-7.08 (m, 2H), 7.15 (t, 1H), 7.23-7.26 (m, 1H), 7.38 (t, 1H), 7.46-7.56 (m, 3H), 7.84 (dd, 1H), 7.93 (dd, 1H), 8.13 (d, 1H), 8.48 (s, 1H).

### Example 095

### 2-(2-Chloro-4-methylphenyl)-N-[4-(3-chlorophenoxy)-3-sulfamoylphenyl]acetamide

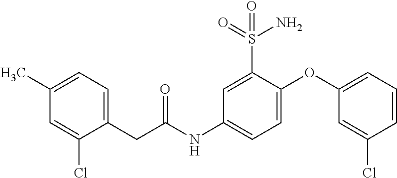 [SMILES: Cc1ccc(CC(=O)Nc2ccc(Oc3cccc(Cl)c3)c(S(N)(=O)=O)c2)c(Cl)c1]

According to general procedures GP3.2 and GP4, purified 5-amino-2-(3-chlorophenoxy)-N-(2,4-dimethoxybenzyl)benzenesulfonamide (50.0 mg, 0.111 mmol) and (2-chloro-4-methylphenyl)acetic acid (22.6 mg, 0.123 mmol) were converted to 2-(2-chloro-4-methylphenyl)-N-[4-(3-chlorophenoxy)-3-sulfamoylphenyl]acetamide and were purified by preparative HPLC (Waters XBrigde C18 5μ 100×30 mm, acetonitrile/water+0.1% formic acid) (23 mg, 0.0494 mmol, 45% yield, 99% purity).

LC-MS (Method A): Rt=1.27 min

MS (ESIpos): m/z=465 (M+H)⁺

¹H-NMR (500 MHz, DICHLOROMETHANE-d₂) δ [ppm]: 2.38 (s, 3H), 3.83 (s, 2H), 5.12 (s, 2H), 6.97 (d, 1H), 7.00 (dd, 1H), 7.10 (t, 1H), 7.14-7.18 (m, 1H), 7.21 (dd, 1H), 7.28-7.38 (m, 4H), 7.83-7.90 (m, 2H).

### Example 096

### 2-(2-Chloro-6-methylphenyl)-N-[4-(3-chlorophenoxy)-3-sulfamoylphenyl]acetamide

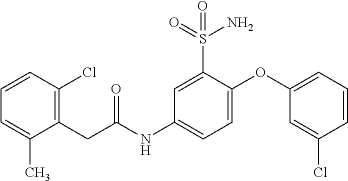 [SMILES: Cc1cccc(Cl)c1CC(=O)Nc1ccc(Oc2cccc(Cl)c2)c(S(N)(=O)=O)c1]

According to general procedures GP3.2 and GP4, purified 5-amino-2-(3-chlorophenoxy)-N-(2,4-dimethoxybenzyl)benzenesulfonamide (50.0 mg, 0.111 mmol) and (2-chloro-6-methylphenyl)acetic acid (22.6 mg, 0.123 mmol) were converted to 2-(2-chloro-6-methylphenyl)-N-[4-(3-chlorophenoxy)-3-sulfamoylphenyl]acetamide and were purified by preparative HPLC (Waters XBrigde C18 5μ 100×30 mm, acetonitrile/water+0.1% formic acid) (15.5 mg, 0.0333 mmol, 30% yield, 98% purity).

LC-MS (Method A): Rt=1.26 min

MS (ESIpos): m/z=465 (M+H)⁺

¹H-NMR (500 MHz, DICHLOROMETHANE-d₂) δ [ppm]: 2.44 (s, 3H), 3.97 (s, 2H), 5.12 (s, 2H), 6.97 (d, 1H), 7.00 (ddd, 1H), 7.10 (t, 1H), 7.19-7.24 (m, 3H), 7.33-7.37 (m, 3H), 7.84 (dd, 1H), 7.90 (d, 1H).

### Example 097

### 2-(2-Chloro-5-methylphenyl)-N-[4-(3-chlorophenoxy)-3-sulfamoylphenyl]acetamide

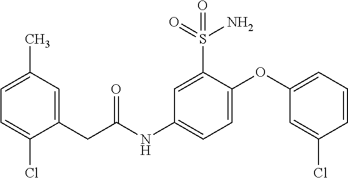 [SMILES: Cc1ccc(Cl)c(CC(=O)Nc2ccc(Oc3cccc(Cl)c3)c(S(N)(=O)=O)c2)c1]

According to general procedures GP3.2 and GP4, purified 5-amino-2-(3-chlorophenoxy)-N-(2,4-dimethoxybenzyl)benzenesulfonamide (50.0 mg, 0.111 mmol) and (2-chloro-5-methylphenyl)acetic acid (22.6 mg, 0.123 mmol) were converted to 2-(2-chloro-5-methylphenyl)-N-[4-(3-chlorophenoxy)-3-sulfamoylphenyl]acetamide and were purified by preparative HPLC (Waters XBrigde C18 5μ 100×30 mm, acetonitrile/water+0.1% formic acid) (19 mg, 0.0408 mmol, 37% yield, 98% purity).

LC-MS (Method A): Rt=1.27 min

MS (ESIpos): m/z=465 (M+H)⁺

¹H-NMR (500 MHz, DICHLOROMETHANE-d₂) δ [ppm]: 2.37 (s, 3H), 3.83 (s, 2H), 5.13 (s, 2H), 6.97 (d, 1H), 7.00 (ddd, 1H), 7.10 (s, 1H), 7.15 (dd, 1H), 7.21 (dd, 1H), 7.25 (d, 1H), 7.32-7.39 (m, 3H), 7.85 (dd, 1H), 7.91 (d, 1H).

### Example 098

### 2-(2-Chloro-3-fluorophenyl)-N-[4-(3-chlorophenoxy)-3-sulfamoylphenyl]acetamide

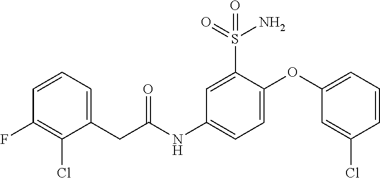 [SMILES: NS(=O)(=O)c1cc(NC(=O)Cc2cccc(F)c2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (50.0 mg, 0.111 mmol) and (2-chloro-3-fluorophenyl)acetic acid (23.1 mg, 0.123 mmol) were converted to 2-(2-chloro-3-fluorophenyl)-N-[4-(3-chlorophenoxy)-3-sulfamoylphenyl]acetamide and were by preparative HPLC (Waters XBrigde C18 5μ 100×30 mm, acetonitrile/water+0.1% formic acid) (19 mg, 0.0405 mmol, 36% yield, 98% purity).

LC-MS (Method A): Rt=1.22 min

MS (ESIpos): m/z=469 (M+H)⁺

¹H-NMR (500 MHz, DICHLOROMETHANE-d₂) δ [ppm]: 3.91 (s, 2H), 5.15 (s., 2H), 6.98 (d, 1H), 7.01 (ddd, 1H), 7.11 (t, 1H), 7.17-7.23 (m, 2H), 7.25 (d, 1H), 7.31-7.38 (m, 2H), 7.40 (s, 1H), 7.85 (dd, 1H), 7.94 (d, 1H).

### Example 099

### 2-(2-Chloro-5-fluorophenyl)-N-[4-(3-chlorophenoxy)-3-sulfamoylphenyl]acetamide

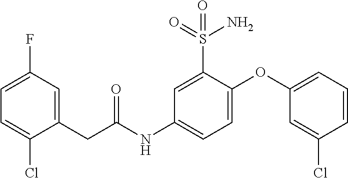 [SMILES: NS(=O)(=O)c1cc(NC(=O)Cc2cc(F)ccc2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (50.0 mg, 0.111 mmol) and (2-chloro-5-fluorophenyl)acetic acid (23.1 mg, 0.123 mmol) were converted to 2-(2-chloro-5-fluorophenyl)-N-[4-(3-chlorophenoxy)-3-sulfamoylphenyl]acetamide and were purified by preparative HPLC (Waters XBrigde C18 5μ 100×30 mm, acetonitrile/water+0.1% formic acid) (16.5 mg, 0.0352 mmol, 31% yield, 99% purity).

LC-MS (Method A): Rt=1.22 min

MS (ESIpos): m/z=469 (M+H)⁺

¹H-NMR (500 MHz, DICHLOROMETHANE-d₂) δ [ppm]: 3.85 (s, 2H), 5.14 (s, 2H), 6.97 (d, 1H), 7.01 (ddd, 1H), 7.04-7.09 (m, 1H), 7.12 (t, 1H), 7.17-7.24 (m, 2H), 7.35 (t, 1H), 7.43 (s, 1H), 7.45 (dd, 1H), 7.86 (dd, 1H), 7.95 (d, 1H).

### Example 100

### 2-(2-Chloro-6-fluorophenyl)-N-[4-(3-chlorophenoxy)-3-sulfamoylphenyl]acetamide

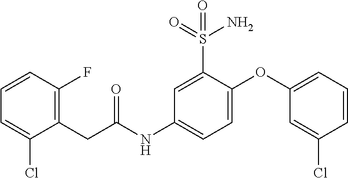 [SMILES: NS(=O)(=O)c1cc(NC(=O)Cc2c(F)cccc2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (50.0 mg, 0.111 mmol) and (2-chloro-6-fluorophenyl)acetic acid (23.1 mg, 0.123 mmol) were converted to 2-(2-chloro-6-fluorophenyl)-N-[4-(3-chlorophenoxy)-3-sulfamoylphenyl]acetamide and were purified by preparative HPLC (Waters XBrigde C18 5μ 100×30 mm, acetonitrile/water+0.1% formic acid) (12 mg, 0.0256 mmol, 23% yield, 98% purity).

LC-MS (Method A): Rt=1.21 min

MS (ESIpos): m/z=469 (M+H)⁺

¹H-NMR (500 MHz, DICHLOROMETHANE-d₂) δ [ppm]: 3.95 (d, 2H), 5.14 (s, 2H), 6.98 (d, 1H), 7.01 (ddd, 1H), 7.10-7.15 (m, 2H), 7.22 (ddd, 1H), 7.30-7.38 (m, 3H), 7.43 (s, 1H), 7.87 (dd, 1H), 7.94 (d, 1H).

### Example 101

### 2-(2-Chloro-6-methoxyphenyl)-N-[4-(3-chlorophenoxy)-3-sulfamoylphenyl]acetamide

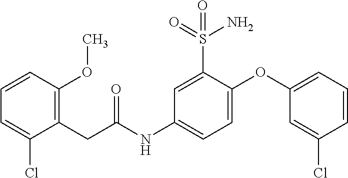 [SMILES: COc1cccc(Cl)c1CC(=O)Nc1ccc(Oc2cccc(Cl)c2)c(S(N)(=O)=O)c1]

According to general procedures GP3.2 and GP4, purified 5-amino-2-(3-chlorophenoxy)-N-(2,4-dimethoxybenzyl)benzenesulfonamide (50.0 mg, 0.111 mmol) and (2-chloro-6-methoxyphenyl)acetic acid (24.6 mg, 0.123 mmol) were converted to 2-(2-chloro-6-methoxyphenyl)-N-[4-(3-chlorophenoxy)-3-sulfamoylphenyl]acetamide and were purified by preparative HPLC (Waters XBrigde C18 5μ 100×30 mm, acetonitrile/water+0.1% formic acid) (14 mg, 0.0291 mmol, 26% yield, 95% purity).

LC-MS (Method A): Rt=1.23 min

MS (ESIpos): m/z=481 (M+H)⁺

¹H-NMR (500 MHz, DICHLOROMETHANE-d₂) δ [ppm]: 3.93 (s, 3H), 3.94 (s, 2H), 5.13 (s, 2H), 6.94 (d, 1H), 6.95-6.98 (m, 1H), 7.00 (ddd, 1H), 7.09-7.13 (m, 2H), 7.20 (ddd, 1H), 7.30 (t, 1H), 7.35 (t, 1H), 7.52 (s, 1H), 7.85-7.88 (m, 2H).

### Example 102

### 2-(2-Chloro-5-methoxyphenyl)-N-[4-(3-chlorophenoxy)-3-sulfamoylphenyl]acetamide

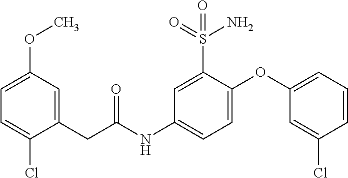 [SMILES: COc1ccc(Cl)c(CC(=O)Nc2ccc(Oc3cccc(Cl)c3)c(S(N)(=O)=O)c2)c1]

According to general procedures GP3.2 and GP4, purified 5-amino-2-(3-chlorophenoxy)-N-(2,4-dimethoxybenzyl)benzenesulfonamide (50.0 mg, 0.111 mmol) and (2-chloro-5-methoxyphenyl)acetic acid (24.6 mg, 0.123 mmol) were converted to 2-(2-chloro-5-methoxyphenyl)-N-[4-(3-chlorophenoxy)-3-sulfamoylphenyl]acetamide and were purified by preparative HPLC (Waters XBrigde C18 5μ 100×30 mm, acetonitrile/water+0.1% formic acid) (15 mg, 0.0312 mmol, 28% yield, 99% purity).

LC-MS (Method A): Rt=1.22 min

MS (ESIpos): m/z=481 (M+H)⁺

¹H-NMR (500 MHz, DICHLOROMETHANE-d₂) δ [ppm]: 3.83 (s, 2H), 3.83 (s, 3H), 5.14 (s, 2H), 6.88 (dd, 1H), 6.95-6.98 (m, 2H), 7.01 (ddd, 1H), 7.11 (t, 1H), 7.21 (ddd, 1H), 7.34 (d, 1H), 7.37 (d, 1H), 7.38 (s, 1H), 7.85 (dd, 1H), 7.91 (d, 1H).

### Example 103

### N-[4-(3-Chlorophenoxy)-3-sulfamoylphenyl]-2-(2,3-dichlorophenyl)acetamide

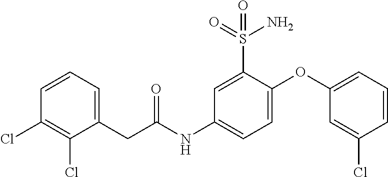 [SMILES: NS(=O)(=O)c1cc(NC(=O)Cc2cccc(Cl)c2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (50.0 mg, 0.111 mmol) and (2,3-dichlorophenyl)acetic acid (25.1 mg, 0.123 mmol) were converted to N-[4-(3-chlorophenoxy)-3-sulfamoylphenyl]-2-(2,3-dichlorophenyl)acetamide and were purified by preparative HPLC (Waters XBrigde C18 5μ 100×30 mm, acetonitrile/water+0.1% formic acid) (13 mg, 0.0268 mmol, 24% yield, 95% purity).

LC-MS (Method A): Rt=1.27 min

MS (ESIpos): m/z=486 (M+H)⁺

¹H-NMR (500 MHz, DICHLOROMETHANE-d₂) δ [ppm]: 3.92 (s, 2H), 5.14 (s, 2H), 6.98 (d, 1H), 7.01 (ddd, 1H), 7.11 (t, 1H), 7.22 (ddd, 1H), 7.30 (t, 1H), 7.33-7.38 (m, 2H), 7.41 (s, 1H), 7.51 (dd, 1H), 7.85 (dd, 1H), 7.95 (d, 1H).

### Example 104

### N-[4-(3-Chlorophenoxy)-3-sulfamoylphenyl]-2-(2,6-dichlorophenyl)acetamide

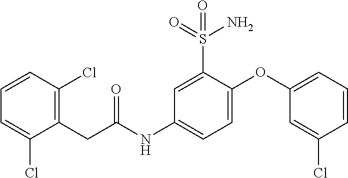 [SMILES: NS(=O)(=O)c1cc(NC(=O)Cc2c(Cl)cccc2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (50.0 mg, 0.111 mmol) and (2,6-dichlorophenyl)acetic acid (25.1 mg, 0.123 mmol) were converted to N-[4-(3-chlorophenoxy)-3-sulfamoylphenyl]-2-(2,6-dichlorophenyl)acetamide and were purified by preparative HPLC (Waters XBrigde C18 5μ 100×30 mm, acetonitrile/water+0.1% formic acid) (11 mg, 0.0268 mmol, 20% yield, 95% purity).

LC-MS (Method A): Rt=1.25 min

MS (ESIpos): m/z=486 (M+H)⁺

¹H-NMR (500 MHz, DICHLOROMETHANE-d₂) δ [ppm]: 4.13 (s, 2H), 5.15 (s, 2H), 6.98 (d, 1H), 7.01 (ddd, 1H), 7.11 (t, 1H), 7.21 (ddd, 1H), 7.29 (dd, 1H), 7.35 (t, 1H), 7.40-7.45 (m, 3H), 7.88 (dd, 1H), 7.93 (d, 1H).

### Example 105

### N-[4-(3-Chlorophenoxy)-3-sulfamoylphenyl]-2-[2-(trifluoromethoxy)phenyl]acetamide

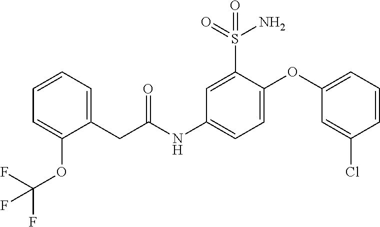 [SMILES: NS(=O)(=O)c1cc(NC(=O)Cc2ccccc2OC(F)(F)F)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and [2-(trifluoromethoxy)phenyl]acetic acid (37.8 mg, 0.172 mmol) were converted to N-[4-(3-chlorophenoxy)-3-sulfamoylphenyl]-2-[2-(trifluoromethoxy)phenyl]acetamide and were purified by preparative HPLC (Waters XBrigde C18 5μ 100×30 mm, acetonitrile/water+0.1% formic acid) (32.5 mg, 0.0649 mmol, 42% yield, 98% purity).

LC-MS (Method A): Rt=1.26 min

MS (ESIpos): m/z=501 (M+H)⁺

¹H-NMR (400 MHz, DICHLOROMETHANE-d₂) δ [ppm]: 3.80 (s, 2H), 5.14 (s, 2H), 6.97 (d, 1H), 7.01 (ddd, 1H), 7.11 (t, 1H), 7.21 (ddd, 1H), 7.32-7.50 (m, 6H), 7.83 (dd, 1H), 7.93 (d, 1H).

### Example 106

### N-[4-(3-Chlorophenoxy)-3-sulfamoylphenyl]-2,2-difluoro-2-phenylacetamide

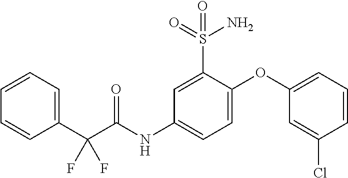 [SMILES: NS(=O)(=O)c1cc(NC(=O)C(F)(F)c2ccccc2)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and difluoro(phenyl)acetic acid (29.5 mg, 0.172 mmol) were converted to N-[4-(3-chlorophenoxy)-3-sulfamoylphenyl]-2,2-difluoro-2-phenylacetamide and were purified by preparative HPLC (Waters XBrigde C18 5μ 100×30 mm, acetonitrile/water+0.1% formic acid) (18 mg, 0.0397 mmol, 25% yield, 98% purity).

LC-MS (Method A): Rt=1.24 min

MS (ESIpos): m/z=453 (M+H)⁺

¹H-NMR (400 MHz, DICHLOROMETHANE-d₂) δ [ppm]: 5.18 (s, 2H), 7.01 (d, 1H), 7.03 (ddd, 1H), 7.13 (t, 1H), 7.24 (ddd, 1H), 7.37 (t, 1H), 7.49-7.61 (m, 3H), 7.67-7.72 (m, 2H), 7.91 (dd, 1H), 8.09 (d, 1H), 8.32 (s, 1H).

### Example 107

### N-[4-(3-Chlorophenoxy)-3-sulfamoylphenyl]-2-[2-chloro-3-(trifluoromethyl)phenyl]acetamide

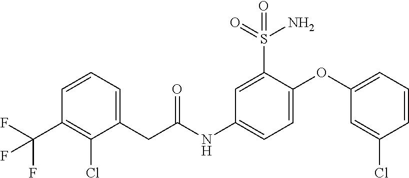 [SMILES: NS(=O)(=O)c1cc(NC(=O)Cc2cccc(C(F)(F)F)c2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and [2-chloro-3-(trifluoromethyl)phenyl]acetic acid (40.9 mg, 0.172 mmol) were converted to N-[4-(3-chlorophenoxy)-3-sulfamoylphenyl]-2-[2-chloro-3-(trifluoromethyl)phenyl]acetamide and were purified by preparative HPLC (Waters XBrigde C18 5μ 100×30 mm, acetonitrile/water+0.1% formic acid) (19 mg, 0.0366 mmol, 23% yield, 98% purity).

LC-MS (Method A): Rt=1.29 min

MS (ESIpos): m/z=519 (M+H)⁺

¹H-NMR (400 MHz, DICHLOROMETHANE-d₂) δ [ppm]: 3.96 (s, 2H), 5.15 (s, 2H), 6.98 (d, 1H), 7.01 (ddd, 1H), 7.11 (t, 1H), 7.22 (ddd, 1H), 7.36 (t, 1H), 7.47 (t, 1H), 7.50 (s, 1H), 7.66 (d, 1H), 7.74 (dd, 1H), 7.85 (dd, 1H), 7.98 (d, 1H).

### Example 108

### N-[4-(3-Chlorophenoxy)-3-sulfamoylphenyl]-2-[2-chloro-6-(trifluoromethyl)phenyl]acetamide

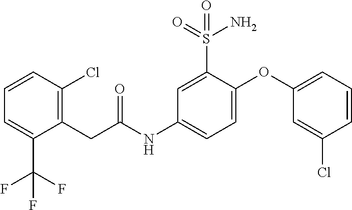 [SMILES: NS(=O)(=O)c1cc(NC(=O)Cc2c(Cl)cccc2C(F)(F)F)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and [2-chloro-6-(trifluoromethyl)phenyl]acetic acid (40.9 mg, 0.172 mmol) were converted to N-[4-(3-chlorophenoxy)-3-sulfamoylphenyl]-2-[2-chloro-6-(trifluoromethyl)phenyl]acetamide and were purified by preparative HPLC (Waters XBrigde C18 5μ 100×30 mm, acetonitrile/water+0.1% formic acid) (28 mg, 0.0549 mmol, 35% yield, 98% purity).

LC-MS (Method A): Rt=1.28 min

MS (ESIpos): m/z=519 (M+H)⁺

¹H-NMR (400 MHz, DICHLOROMETHANE-d₂) δ [ppm]: 4.12 (s, 2H), 5.14 (s, 2H), 6.98 (d, 1H), 7.01 (ddd, 1H), 7.11 (t, 1H), 7.21 (ddd, 1H), 7.35 (t, 1H), 7.37 (s, 1H), 7.47 (t, 1H), 7.72 (dd, 2H), 7.86 (dd, 1H), 7.92 (d, 1H).

### Example 109

### N-[4-(3-Chlorophenoxy)-3-sulfamoylphenyl]-2-[2-chloro-5-(trifluoromethyl)phenyl]acetamide

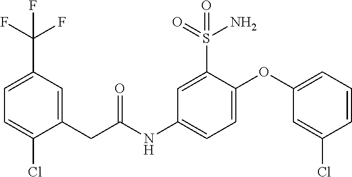 [SMILES: NS(=O)(=O)c1cc(NC(=O)Cc2cc(C(F)(F)F)ccc2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and [2-chloro-5-(trifluoromethyl)phenyl]acetic acid (40.9 mg, 0.172 mmol) were converted to N-[4-(3-chlorophenoxy)-3-sulfamoylphenyl]-2-[2-chloro-5-(trifluoromethyl)phenyl]acetamide and were purified by preparative HPLC (Waters XBrigde C18 5μ 100×30 mm, acetonitrile/water+0.1% formic acid) (34 mg, 0.0655 mmol, 42% yield, 98% purity).

LC-MS (Method A): Rt=1.31 min

MS (ESIpos): m/z=519 (M+H)⁺

¹H-NMR (400 MHz, DICHLOROMETHANE-d₂) δ [ppm]: 3.93 (s, 2H), 5.15 (s, 2H), 6.98 (d, 1H), 7.01 (ddd, 1H), 7.11 (t, 1H), 7.22 (ddd, 1H), 7.36 (t, 1H), 7.50 (s, 1H), 7.56-7.64 (m, 2H), 7.70-7.72 (m, 1H), 7.87 (dd, 1H), 7.96 (d, 1H).

### Example 110

### N-[4-(3-Chlorophenoxy)-3-sulfamoylphenyl]-2-(2,4-dichlorophenyl)acetamide

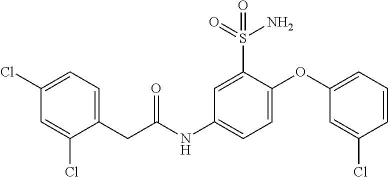 [SMILES: NS(=O)(=O)c1cc(NC(=O)Cc2ccc(Cl)cc2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and (2,4-dichlorophenyl)acetic acid (35.2 mg, 0.172 mmol) were converted to N-[4-(3-chlorophenoxy)-3-sulfamoylphenyl]-2-(2,4-dichlorophenyl)acetamide and were purified by preparative HPLC (Waters XBrigde C18 5μ 100×30 mm, acetonitrile/water+0.1% formic acid) (31 mg, 0.0638 mmol, 41% yield, 98% purity).

LC-MS (Method A): Rt=1.30 min

MS (ESIpos): m/z=485 (M+H)⁺

¹H-NMR (400 MHz, DICHLOROMETHANE-d₂) δ [ppm]: 3.84 (s, 2H), 5.16 (s, 2H), 6.97 (d, 1H), 7.01 (ddd, 1H), 7.11 (t, 1H), 7.21 (ddd, 1H), 7.31-7.40 (m, 3H), 7.45 (s, 1H), 7.50 (d, 1H), 7.84 (dd, 1H), 7.94 (d, 1H).

### Example 111

### N-[4-(3-Chlorophenoxy)-3-sulfamoylphenyl]-2-(4,6-dichloropyridin-3-yl)acetamide

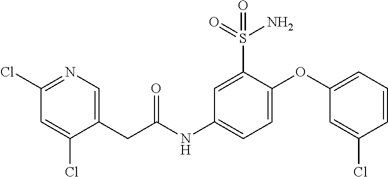 [SMILES: NS(=O)(=O)c1cc(NC(=O)Cc2cnc(Cl)cc2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and (4,6-dichloropyridin-3-yl)acetic acid (35.3 mg, 0.172 mmol) were converted to N-[4-(3-chlorophenoxy)-3-sulfamoylphenyl]-2-(4,6-dichloropyridin-3-yl)acetamide and were purified by preparative HPLC (Waters XBrigde C18 5μ 100×30 mm, acetonitrile/water+0.1% formic acid) (29.5 mg, 0.0606 mmol, 39% yield, 98% purity).

LC-MS (Method A): Rt=1.17 min

MS (ESIpos): m/z=486 (M+H)⁺

¹H-NMR (400 MHz, DICHLOROMETHANE-d₂) δ [ppm]: 3.85 (s, 2H), 5.22 (s, 2H), 6.98 (d, 1H), 7.01 (ddd, 1H), 7.11 (t, 1H), 7.22 (ddd, 1H), 7.36 (t, 1H), 7.47 (s, 1H), 7.65 (s, 1H), 7.87 (dd, 1H), 7.95 (d, 1H), 8.37 (s, 1H).

### Example 112

### N-[4-(3-Chlorophenoxy)-3-sulfamoylphenyl]-2-(3-chloropyridin-2-yl)acetamide

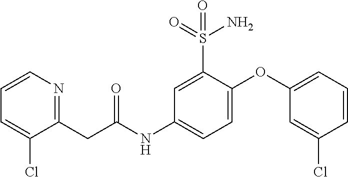 [SMILES: NS(=O)(=O)c1cc(NC(=O)Cc2ncccc2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and (3-chloropyridin-2-yl)acetic acid (29.4 mg, 0.172 mmol) were converted to N-[4-(3-chlorophenoxy)-3-sulfamoylphenyl]-2-(3-chloropyridin-2-yl)acetamide and were purified by preparative HPLC (Waters XBrigde C18 5μ 100×30 mm, acetonitrile/water+0.1% formic acid) (21 mg, 0.0464 mmol, 30% yield, 99% purity).

LC-MS (Method A): Rt=1.07 min

MS (ESIpos): m/z=452 (M+H)⁺

¹H-NMR (400 MHz, DMSO-d₆) δ [ppm]: 4.04 (s, 2H), 6.98 (ddd, 1H), 7.08-7.13 (m, 2H), 7.20 (ddd, 1H), 7.36-7.43 (m, 4H), 7.79 (dd, 1H), 7.95 (dd, 1H), 8.23 (d, 1H), 8.49 (dd, 1H), 10.59 (s, 1H).

### Example 113

### N-[4-(3-Chlorophenoxy)-3-sulfamoylphenyl]-2-[2-(difluoromethoxy)phenyl]acetamide

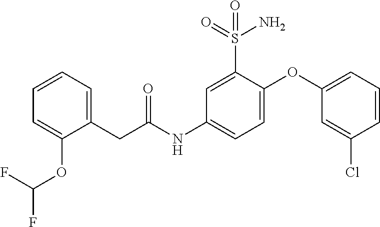 [SMILES: NS(=O)(=O)c1cc(NC(=O)Cc2ccccc2OC(F)F)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and [2-(difluoromethoxy)phenyl]acetic acid (34.7 mg, 0.172 mmol) were converted to N-[4-(3-chlorophenoxy)-3-sulfamoylphenyl]-2-[2-(difluoromethoxy)phenyl]acetamide and were purified by preparative HPLC (Waters XBrigde C18 5μ 100×30 mm, acetonitrile/water+0.1% formic acid) (30 mg, 0.0621 mmol, 40% yield, 99% purity).

LC-MS (Method A): Rt=1.20 min

MS (ESIpos): m/z=483 (M+H)⁺

¹H-NMR (400 MHz, DICHLOROMETHANE-d₂) δ [ppm]: 3.80 (s, 2H), 5.17 (s, 2H), 6.66 (t, 1H), 6.98 (d, 1H), 7.02 (ddd, 1H), 7.12 (t, 1H), 7.20-7.25 (m, 2H), 7.27-7.47 (m, 4H), 7.53 (s, 1H), 7.84 (dd, 1H), 7.96 (d, 1H).

### Example 114

### N-[4-(3-Chlorophenoxy)-3-sulfamoylphenyl]-2-(2,5-dichlorophenyl)acetamide

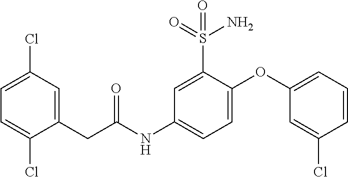 [SMILES: NS(=O)(=O)c1cc(NC(=O)Cc2cc(Cl)ccc2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and (2,5-dichlorophenyl)acetic acid (35.2 mg, 0.172 mmol) were converted to N-[4-(3-chlorophenoxy)-3-sulfamoylphenyl]-2-(2,5-dichlorophenyl)acetamide and were purified by preparative HPLC (Waters XBrigde C18 5μ 100×30 mm, acetonitrile/water+0.1% formic acid) (28 mg, 0.0576 mmol, 37% yield, 99% purity).

LC-MS (Method A): Rt=1.28 min

MS (ESIpos): m/z=485 (M+H)⁺

¹H-NMR (400 MHz, DICHLOROMETHANE-d₂) δ [ppm]: 3.86 (s, 2H), 5.18 (s, 2H), 6.99 (d, 1H), 7.02 (ddd, 1H), 7.13 (t, 1H), 7.23 (ddd, 1H), 7.33 (dd, 1H), 7.37 (t, 1H), 7.41-7.47 (m, 2H), 7.49 (s, 1H), 7.88 (dd, 1H), 7.96 (d, 1H).

### Example 115

### 2-[6-Chloro-2,3-difluoro-4-(trifluoromethyl)phenyl]-N-[4-(3-chlorophenoxy)-3-sulfamoylphenyl]acetamide

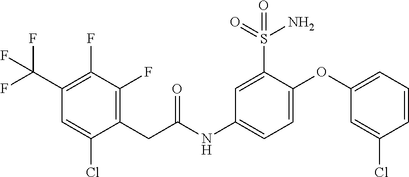 [SMILES: NS(=O)(=O)c1cc(NC(=O)Cc2c(Cl)cc(C(F)(F)F)c(F)c2F)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and [6-chloro-2,3-difluoro-4-(trifluoromethyl)phenyl]acetic acid (47.1 mg, 0.172 mmol) were converted to 2-[6-chloro-2,3-difluoro-4-(trifluoromethyl)phenyl]-N-[4-(3-chlorophenoxy)-3-sulfamoylphenyl]acetamide and were purified by preparative HPLC (Waters XBrigde C18 5μ 100×30 mm, acetonitrile/water+0.1% formic acid) (14 mg, 0.0252 mmol, 16% yield, 99% purity).

LC-MS (Method A): Rt=1.36 min

MS (ESIpos): m/z=555 (M+H)⁺

¹H-NMR (400 MHz, DMSO-d₆) δ [ppm]: 4.05 (s, 2H), 6.99 (ddd, 1H), 7.08-7.13 (m, 2H), 7.21 (ddd, 1H), 7.37-7.45 (m, 3H), 7.77 (dd, 1H), 7.88-7.92 (m, 1H), 8.20 (d, 1H), 10.75 (s, 1H).

### Example 116

### N-[4-(3-Chlorophenoxy)-3-sulfamoylphenyl]-2-[4-(trifluoromethyl)phenyl]acetamide

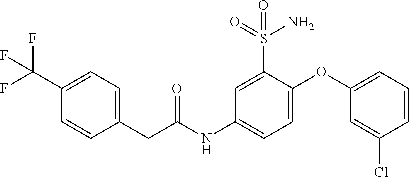 [SMILES: NS(=O)(=O)c1cc(NC(=O)Cc2ccc(C(F)(F)F)cc2)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and [4-(trifluoromethyl)phenyl]acetic acid (35.0 mg, 0.172 mmol) were converted to N-[4-(3-chlorophenoxy)-3-sulfamoylphenyl]-2-(2,5-dichlorophenyl)acetamide and were purified by preparative HPLC (Waters XBrigde C18 5μ 100×30 mm, acetonitrile/water+0.1% formic acid) (28 mg, 0.0577 mmol, 37% yield, 99% purity).

LC-MS (Method A): Rt=1.27 min

MS (ESIpos): m/z=485 (M+H)⁺

¹H-NMR (400 MHz, DICHLOROMETHANE-d₂) δ [ppm]: 3.83 (s, 2H), 5.20 (s., 2H), 6.98 (d, 1H), 7.02 (dd, 1H), 7.12 (t, 1H), 7.23 (ddd, 1H), 7.37 (t, 1H), 7.47 (s, 1H), 7.53 (d, 2H), 7.69 (d, 2H), 7.87 (dd, 1H), 7.96 (d, 1H).

### Example 117

### 2-(5-Bromo-2-chlorophenyl)-N-[4-(3-chlorophenoxy)-3-sulfamoylphenyl]acetamide

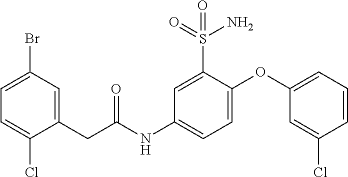 [SMILES: NS(=O)(=O)c1cc(NC(=O)Cc2cc(Br)ccc2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and (5-bromo-2-chlorophenyl)acetic acid (42.8 mg, 0.172 mmol) were converted to 2-(5-bromo-2-chlorophenyl)-N-[4-(3-chlorophenoxy)-3-sulfamoylphenyl]acetamide and were purified by preparative HPLC (Waters XBrigde C18 5μ 100×30 mm, acetonitrile/water+0.1% formic acid) (14 mg, 0.0264 mmol, 17% yield, 99% purity).

LC-MS (Method A): Rt=1.30 min

MS (ESIpos): m/z=531 (M+H)⁺

¹H-NMR (400 MHz, DMSO-d₆) δ [ppm]: 3.87 (s, 2H), 6.98 (ddd, 1H), 7.08-7.12 (m, 2H), 7.20 (ddd, 1H), 7.36-7.45 (m, 4H), 7.53 (dd, 1H), 7.70 (d, 1H), 7.79 (dd, 1H), 8.21 (d, 1H), 10.57 (s, 1H).

### Example 118

### 2-(4-Bromo-2-chloro-5-methylphenyl)-N-[4-(3-chlorophenoxy)-3-sulfamoylphenyl]acetamide

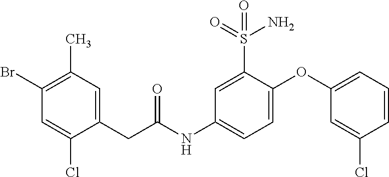 [SMILES: Cc1cc(CC(=O)Nc2ccc(Oc3cccc(Cl)c3)c(S(N)(=O)=O)c2)c(Cl)cc1Br]

According to general procedures GP3.2 and GP4, purified 5-amino-2-(3-chlorophenoxy)-N-(2,4-dimethoxybenzyl)benzenesulfonamide (70.0 mg, 0.156 mmol) and (4-bromo-2-chloro-5-methylphenyl)acetic acid (45.2 mg, 0.172 mmol) were converted to 2-(4-bromo-2-chloro-5-methylphenyl)-N-[4-(3-chlorophenoxy)-3-sulfamoylphenyl]acetamide and were purified by preparative HPLC (Waters XBrigde C18 5μ 100×30 mm, acetonitrile/water+0.1% formic acid) (16 mg, 0.0294 mmol, 19% yield, 98% purity).

LC-MS (Method A): Rt=1.38 min

MS (ESIpos): m/z=545 (M+H)⁺

¹H-NMR (400 MHz, DICHLOROMETHANE-d₂) δ [ppm]: 2.43 (s, 3H), 3.81 (s, 2H), 5.17 (s, 2H), 6.98 (d, 1H), 7.02 (ddd, 1H), 7.12 (t, 1H), 7.23 (ddd, 1H), 7.31-7.40 (m, 2H), 7.43 (s, 1H), 7.67 (s, 1H), 7.87 (dd, 1H), 7.94 (d, 1H).

### Example 119

### N-[4-(3-Chlorophenoxy)-3-sulfamoylphenyl]-2-(3-chloropyridin-4-yl)acetamide

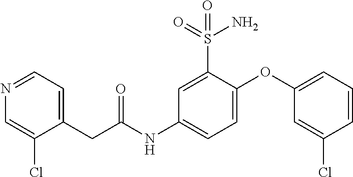 [SMILES: NS(=O)(=O)c1cc(NC(=O)Cc2ccncc2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and (3-chloropyridin-4-yl)acetic acid (35.7 mg, 0.172 mmol) were converted to N-[4-(3-chlorophenoxy)-3-sulfamoylphenyl]-2-(3-chloropyridin-4-yl)acetamide and were purified by preparative HPLC (Waters XBrigde C18 5μ 100×30 mm, acetonitrile/water+0.1% formic acid) (25.8 mg, 0.0570 mmol, 37% yield, 99% purity).

LC-MS (Method A): Rt=1.03 min

MS (ESIpos): m/z=452 (M+H)⁺

¹H-NMR (400 MHz, DMSO-d₆) δ [ppm]: 3.93 (s, 2H), 6.98 (dd, 1H), 7.08-7.12 (m, 2H), 7.20 (ddd, 1H), 7.38-7.44 (m, 3H), 7.50 (d, 1H), 7.79 (dd, 1H), 8.21 (d, 1H), 8.50 (d, 1H), 8.63 (s, 1H), 10.64 (s, 1H).

### Example 120

### 2-(2-Chloro-6-fluoro-3-methylphenyl)-N-[4-(3-chlorophenoxy)-3-sulfamoylphenyl]acetamide

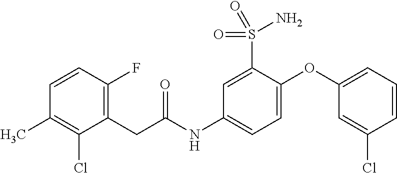 [SMILES: Cc1ccc(F)c(CC(=O)Nc2ccc(Oc3cccc(Cl)c3)c(S(N)(=O)=O)c2)c1Cl]

According to general procedures GP3.2 and GP4, purified 5-amino-2-(3-chlorophenoxy)-N-(2,4-dimethoxybenzyl)benzenesulfonamide (70.0 mg, 0.156 mmol) and (2-chloro-6-fluoro-3-methylphenyl)acetic acid (34.8 mg, 0.172 mmol) were converted to 2-(2-chloro-6-fluoro-3-methylphenyl)-N-[4-(3-chlorophenoxy)-3-sulfamoylphenyl]acetamide and were purified by preparative HPLC (Waters XBrigde C18 5μ 100×30 mm, acetonitrile/water+0.1% formic acid) (9.5 mg, 0.0197 mmol, 13% yield, 97% purity).

LC-MS (Method A): Rt=1.27 min

MS (ESIpos): m/z=483 (M+H)⁺

¹H-NMR (400 MHz, DMSO-d₆) δ [ppm]: 2.33 (s, 3H), 3.91 (d, 2H), 6.98 (ddd, 1H), 7.07-7.12 (m, 2H), 7.14-7.23 (m, 2H), 7.35 (dd, 1H), 7.38-7.43 (m, 3H), 7.78 (dd, 1H), 8.21 (d, 1H), 10.62 (s, 1H).

### Example 121

### 2-(6-Chloro-2-fluoro-3-methylphenyl)-N-[4-(3-chlorophenoxy)-3-sulfamoylphenyl]acetamide

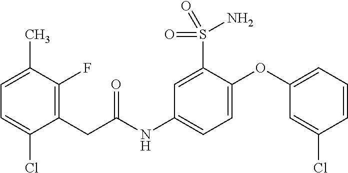 [SMILES: Cc1ccc(Cl)c(CC(=O)Nc2ccc(Oc3cccc(Cl)c3)c(S(N)(=O)=O)c2)c1F]

According to general procedures GP3.2 and GP4, purified 5-amino-2-(3-chlorophenoxy)-N-(2,4-dimethoxybenzyl)benzenesulfonamide (70.0 mg, 0.156 mmol) and (6-chloro-2-fluoro-3-methylphenyl)acetic acid (34.8 mg, 0.172 mmol) were converted 2-(6-chloro-2-fluoro-3-methylphenyl)-N-[4-(3-chlorophenoxy)-3-sulfamoylphenyl]acetamide and were purified by preparative HPLC (Waters XBrigde C18 5μ 100×30 mm, acetonitrile/water+0.1% formic acid) (24.7 mg, 0.0511 mmol, 33% yield, 97% purity).

LC-MS (Method A): Rt=1.28 min

MS (ESIneg): m/z=481 (M−H)⁺

¹H-NMR (400 MHz, DMSO-d₆) δ [ppm]: 2.24 (d, 3H), 3.89 (d, 2H), 6.98 (ddd, 1H), 7.08-7.12 (m, 2H), 7.20 (ddd, 1H), 7.22-7.29 (m, 2H), 7.37-7.43 (m, 3H), 7.78 (dd, 1H), 8.21 (d, 1H), 10.62 (s, 1H).

### Example 122

### 2-(2-Chloro-3,6-difluorophenyl)-N-[4-(3-chlorophenoxy)-3-sulfamoylphenyl]acetamide

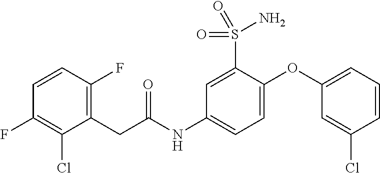 [SMILES: NS(=O)(=O)c1cc(NC(=O)Cc2c(F)ccc(F)c2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and (2-chloro-3,6-difluorophenyl)acetic acid (35.4 mg, 0.172 mmol) were converted to 2-(2-chloro-3,6-difluorophenyl)-N-[4-(3-chlorophenoxy)-3-sulfamoylphenyl]acetamide and were purified by preparative HPLC (Waters XBrigde C18 5μ 100×30 mm, acetonitrile/water+0.1% formic acid) (33.6 mg, 0.0600 mmol, 44% yield, 99% purity).

LC-MS (Method A): Rt=1.23 min

MS (ESIneg): m/z=485 (M+H)⁻

¹H-NMR (400 MHz, DMSO-d₆) δ [ppm]: 3.94 (d, 2H), 6.98 (ddd, 1H), 7.08-7.12 (m, 2H), 7.20 (ddd, 1H), 7.31-7.50 (m, 5H), 7.77 (dd, 1H), 8.20 (d, 1H), 10.67 (s, 1H).

### Example 123

### 2-(2-Chloro-4,5-difluorophenyl)-N-[4-(3-chlorophenoxy)-3-sulfamoylphenyl]acetamide

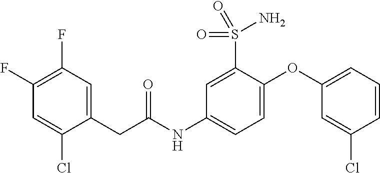 [SMILES: NS(=O)(=O)c1cc(NC(=O)Cc2cc(F)c(F)cc2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and (6-chloro-2,3-difluorophenyl)acetic acid (35.4 mg, 0.172 mmol) were converted to 2-(2-chloro-4,5-difluorophenyl)-N-[4-(3-chlorophenoxy)-3-sulfamoylphenyl]acetamide and were purified by preparative HPLC (Waters XBrigde C18 5μ 100×30 mm, acetonitrile/water+0.1% formic acid) (19.5 mg, 0.0426 mmol, 27% yield, 98% purity).

LC-MS (Method A): Rt=1.25 min

MS (ESIpos): m/z=487 (M+H)⁺

¹H-NMR (400 MHz, DMSO-d₆) δ [ppm]: 3.85 (s, 2H), 6.98 (ddd, 1H), 7.08-7.11 (m, 2H), 7.20 (ddd, 1H), 7.37-7.43 (m, 3H), 7.62 (dd, 1H), 7.74 (dd, 1H), 7.78 (dd, 1H), 8.20 (d, 1H), 10.54 (s, 1H).

### Example 124

### N-[4-(3-Chlorophenoxy)-3-sulfamoylphenyl]-2-(2,3-dichloro-6-fluorophenyl)acetamide

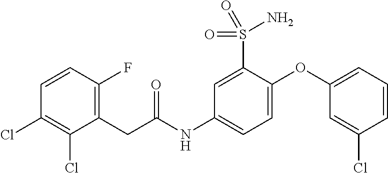 [SMILES: NS(=O)(=O)c1cc(NC(=O)Cc2c(F)ccc(Cl)c2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and (2,3-dichloro-6-fluorophenyl)acetic acid (38.3 mg, 0.172 mmol) were converted to N-[4-(3-chlorophenoxy)-3-sulfamoylphenyl]-2-(2,3-dichloro-6-fluorophenyl)acetamide and were purified by preparative HPLC (Waters XBrigde C18 5μ 100×30 mm, acetonitrile/water+0.1% formic acid) (17.9 mg, 0.0355 mmol, 23% yield, 98% purity).

LC-MS (Method A): Rt=1.28 min

MS (ESIneg): m/z=501 (M−H)⁺

¹H-NMR (400 MHz, DMSO-d₆) δ [ppm]: 3.97 (d, 2H), 6.98 (ddd, 1H), 7.08-7.12 (m, 2H), 7.20 (ddd, 1H), 7.33-7.43 (m, 4H), 7.68 (dd, 1H), 7.77 (dd, 1H), 8.20 (d, 1H), 10.67 (s, 1H).

### Example 125

### N-[4-(3-Chlorophenoxy)-3-sulfamoylphenyl]-2-(2,3,6-trichlorophenyl)acetamide

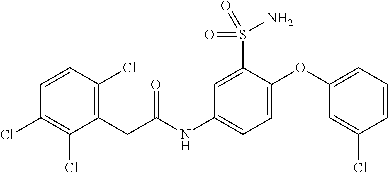 [SMILES: NS(=O)(=O)c1cc(NC(=O)Cc2c(Cl)ccc(Cl)c2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and (2,3,6-trichlorophenyl)acetic acid (41.1 mg, 0.172 mmol) were converted to N-[4-(3-chlorophenoxy)-3-sulfamoylphenyl]-2-(2,3,6-trichlorophenyl)acetamide and were purified by preparative HPLC (Waters XBrigde C18 5μ 100×30 mm, acetonitrile/water+0.1% formic acid) (5.9 mg, 0.0113 mmol, 7% yield, 98% purity).

LC-MS (Method A): Rt=1.32 min

MS (ESIpos): m/z=520 (M+H)⁺

¹H-NMR (400 MHz, DMSO-d₆) δ [ppm]: 4.14 (s, 2H), 6.98 (ddd, 1H), 7.08-7.12 (m, 2H), 7.20 (ddd, 1H), 7.38-7.43 (m, 3H), 7.56 (d, 1H), 7.66 (d, 1H), 7.77 (dd, 1H), 8.22 (d, 1H), 10.67 (s, 1H).

### Example 126

### N-[4-(3-Chlorophenoxy)-3-sulfamoylphenyl]-2-(2,6-dichloro-4-methylphenyl)acetamide

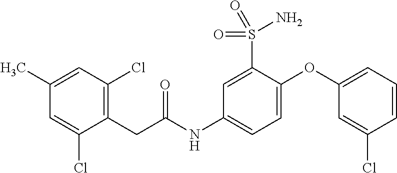 [SMILES: Cc1cc(Cl)c(CC(=O)Nc2ccc(Oc3cccc(Cl)c3)c(S(N)(=O)=O)c2)c(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and (2,6-dichloro-4-methylphenyl)acetic acid (37.8 mg, 0.172 mmol) were converted to N-[4-(3-chlorophenoxy)-3-sulfamoylphenyl]-2-(2,6-dichloro-4-methylphenyl)acetamide and were purified by preparative HPLC (Waters XBrigde C18 5μ 100×30 mm, acetonitrile/water+0.1% formic acid) (1.4 mg, 0.00280 mmol, 2% yield, 95% purity).

LC-MS (Method A): Rt=1.33 min

MS (ESIpos): m/z=501 (M+H)⁺

¹H-NMR (400 MHz, DMSO-d₆) δ [ppm]: 2.32 (s, 3H), 4.00 (s, 2H), 6.98 (ddd, 1H), 7.08-7.12 (m, 2H), 7.20 (ddd, 1H), 7.34 (s, 2H), 7.37-7.42 (m, 3H), 7.77 (dd, 1H), 8.21 (d, 1H), 10.59 (s, 1H).

### Example 127

### N-[4-(3-chlorophenoxy)-3-sulfamoylphenyl]-2-[2,3-dichloro-6-(trifluoromethyl)phenyl]acetamide

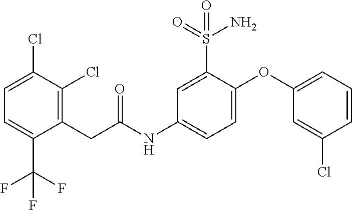 [SMILES: NS(=O)(=O)c1cc(NC(=O)Cc2c(C(F)(F)F)ccc(Cl)c2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and [2,3-dichloro-6-(trifluoromethyl)phenyl]acetic acid (46.8 mg, 0.172 mmol) were converted to N-[4-(3-chlorophenoxy)-3-sulfamoylphenyl]-2-[2,3-dichloro-6-(trifluoromethyl)phenyl]acetamide and were purified by preparative HPLC (Waters XBrigde C18 5μ 100×30 mm, acetonitrile/water+0.1% formic acid) (11 mg, 0.0199 mmol, 13% yield, 97% purity).

LC-MS (Method A): Rt=1.35 min

MS (ESIpos): m/z=553 (M+H)⁺

¹H-NMR (400 MHz, DMSO-d₆) δ [ppm]: 4.13 (s, 2H), 6.98 (ddd, 1H), 7.08-7.12 (m, 2H), 7.20 (ddd, 1H), 7.40 (d, 3H), 7.73 (dd, 1H), 7.80 (d, 1H), 7.87 (d, 1H), 8.21 (d, 1H), 10.66 (s, 1H).

### Example 128

### N-[4-(3-Chlorophenoxy)-3-sulfamoylphenyl]-2-(2,6-dichloro-3-methylphenyl)acetamide

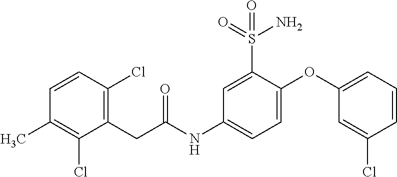 [SMILES: Cc1ccc(Cl)c(CC(=O)Nc2ccc(Oc3cccc(Cl)c3)c(S(N)(=O)=O)c2)c1Cl]

According to general procedures GP3.2 and GP4, purified 5-amino-2-(3-chlorophenoxy)-N-(2,4-dimethoxybenzyl)benzenesulfonamide (70.0 mg, 0.156 mmol) and (2,6-dichloro-3-methylphenyl)acetic acid (37.6 mg, 0.172 mmol) were converted to N-[4-(3-chlorophenoxy)-3-sulfamoylphenyl]-2-(2,6-dichloro-3-methylphenyl)acetamide and were purified by preparative HPLC (Waters XBrigde C18 5μ 100×30 mm, acetonitrile/water+0.1% formic acid) (17 mg, 0.0340 mmol, 22% yield, 97% purity).

LC-MS (Method A): Rt=1.31 min

MS (ESIpos): m/z=499 (M+H)⁺

¹H-NMR (400 MHz, DMSO-d₆) δ [ppm]: 2.35 (s, 3H), 4.08 (s, 2H), 6.99 (ddd, 1H), 7.08-7.12 (m, 2H), 7.20 (ddd, 1H), 7.34 (dd, 1H), 7.37-7.43 (m, 4H), 7.77 (dd, 1H), 8.22 (d, 1H), 10.63 (s, 1H).

### Example 129

### N-[4-(3-Chlorophenoxy)-3-sulfamoylphenyl]-2-(2,6-dichloro-3-cyclopropylphenyl)acetamide

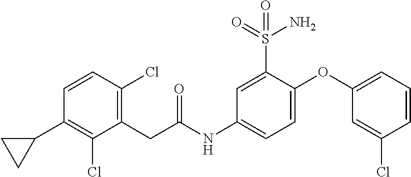 [SMILES: NS(=O)(=O)c1cc(NC(=O)Cc2c(Cl)ccc(C3CC3)c2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and (2,6-dichloro-3-cyclopropylphenyl)acetic acid (42.0 mg, 0.172 mmol) were converted to N-[4-(3-chlorophenoxy)-3-sulfamoylphenyl]-2-(2,6-dichloro-3-cyclopropylphenyl)acetamide and were purified by preparative HPLC (Waters XBrigde C18 5μ 100×30 mm, acetonitrile/water+0.1% formic acid) (12 mg, 0.0228 mmol, 15% yield, 97% purity).

LC-MS (Method A): Rt=1.37 min

MS (ESIpos): m/z=525 (M+H)⁺

¹H-NMR (400 MHz, DMSO-d₆) δ [ppm]: 0.69-0.73 (m, 2H), 0.99-1.04 (m, 2H), 2.11-2.17 (m, 1H), 4.09 (s, 2H), 6.98 (ddd, 1H), 7.02 (d, 1H), 7.07-7.10 (m, 2H), 7.19 (ddd, 1H), 7.36-7.41 (m, 4H), 7.77 (dd, 1H), 8.22 (d, 1H), 10.63 (s, 1H).

### Example 130

### N-[4-(3-Chlorophenoxy)-3-sulfamoylphenyl]-2-[2,6-dichloro-3-(trifluoromethyl)phenyl]acetamide

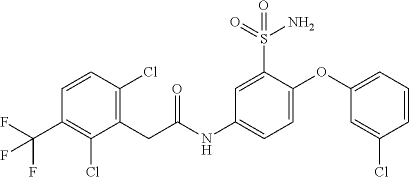 [SMILES: NS(=O)(=O)c1cc(NC(=O)Cc2c(Cl)ccc(C(F)(F)F)c2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and [2,6-dichloro-3-(trifluoromethyl)phenyl]acetic acid (46.8 mg, 0.172 mmol) were converted to N-[4-(3-chlorophenoxy)-3-sulfamoylphenyl]-2-[2,6-dichloro-3-(trifluoromethyl)phenyl]acetamide and were purified by preparative HPLC (Waters XBrigde C18 5μ 100×30 mm, acetonitrile/water+0.1% formic acid) (43 mg, 0.0777 mmol, 50% yield, 99% purity).

LC-MS (Method A): Rt=1.34 min

MS (ESIpos): m/z=553 (M+H)⁺

¹H-NMR (400 MHz, DMSO-d₆) δ [ppm]: 4.20 (s, 2H), 6.99 (ddd, 1H), 7.08-7.13 (m, 2H), 7.20 (ddd, 1H), 7.40 (t, 1H), 7.42 (s, 2H), 7.73-7.79 (m, 2H), 7.86 (d, 1H), 8.22 (d, 1H), 10.73 (s, 1H).

### Example 131

### 2-(3-bromo-2,6-dichlorophenyl)-N-[4-(3-chlorophenoxy)-3-sulfamoylphenyl]acetamide

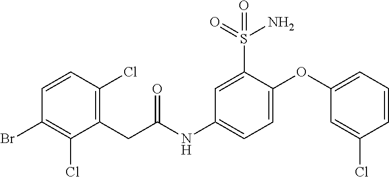 [SMILES: NS(=O)(=O)c1cc(NC(=O)Cc2c(Cl)ccc(Br)c2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and (3-bromo-2,6-dichlorophenyl)acetic acid (49.1 mg, 0.172 mmol) were converted to N-[4-(3-chlorophenoxy)-3-sulfamoylphenyl]-2-[2,6-dichloro-3-(trifluoromethyl)phenyl]acetamide and were purified by preparative HPLC (Waters XBrigde C18 5μ 100×30 mm, acetonitrile/water+0.2% aqueous ammonia (32%)) (32 mg, 0.0567 mmol, 36% yield, 95% purity).

LC-MS (Method B): Rt=1.33 min

MS (ESIneg): m/z=563 (M−H)⁺

¹H-NMR (400 MHz, DMSO-d₆) δ [ppm]: 4.16 (s, 2H), 6.99 (ddd, 1H), 7.08-7.12 (m, 2H), 7.20 (ddd, 1H), 7.37-7.43 (m, 3H), 7.48 (d, 1H), 7.74-7.80 (m, 2H), 8.21 (d, 1H), 10.67 (m, 1H).

### Example 132

### 2-(3-Bromo-2-chloro-6-methylphenyl)-N-[4-(3-chlorophenoxy)-3-sulfamoylphenyl]acetamide

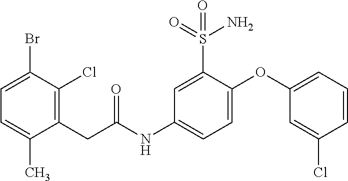 [SMILES: Cc1ccc(Br)c(Cl)c1CC(=O)Nc1ccc(Oc2cccc(Cl)c2)c(S(N)(=O)=O)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and (3-bromo-2-chloro-6-methylphenyl)acetic acid (45.2 mg, 0.172 mmol) were converted to 2-(3-bromo-2-chloro-6-methylphenyl)-N-[4-(3-chlorophenoxy)-3-sulfamoylphenyl]acetamide and were purified by preparative HPLC (Waters XBrigde C18 5μ 100×30 mm, acetonitrile/water+0.2% aqueous ammonia (32%)) (43 mg, 0.0777 mmol, 50% yield, 99% purity).

LC-MS (Method B): Rt=1.32 min

MS (ESIneg): m/z=543 (M−H)⁺

¹H-NMR (400 MHz, DMSO-d₆) δ [ppm]: 2.31 (s, 3H), 4.01 (s, 2H), 6.98 (ddd, 1H), 7.07-7.12 (m, 2H), 7.17 (dd, 1H), 7.20 (ddd, 1H), 7.37-7.44 (m, 3H), 7.60 (d, 1H), 7.78 (dd, 1H), 8.22 (d, 1H), 10.61 (s, 1H).

### Example 133

### 2-(3-Bromo-6-chloro-2-methylphenyl)-N-[4-(3-chlorophenoxy)-3-sulfamoylphenyl]acetamide

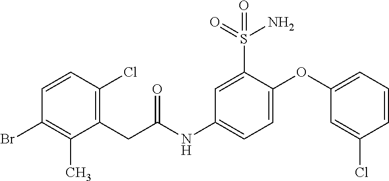 [SMILES: Cc1c(Br)ccc(Cl)c1CC(=O)Nc1ccc(Oc2cccc(Cl)c2)c(S(N)(=O)=O)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and (3-bromo-6-chloro-2-methylphenyl)acetic acid (45.2 mg, 0.172 mmol) were converted to 2-(3-bromo-6-chloro-2-methylphenyl)-N-[4-(3-chlorophenoxy)-3-sulfamoylphenyl]acetamide and were purified by preparative HPLC (Waters XBrigde C18 5μ 100×30 mm, acetonitrile/water+0.2% aqueous ammonia (32%)) (25 mg, 0.0459 mmol, 29% yield, 90% purity).

LC-MS (Method B): Rt=1.34 min

MS (ESIneg): m/z=543 (M−H)⁺

¹H-NMR (400 MHz, DMSO-d₆) δ [ppm]: 2.42 (s, 3H), 4.05 (m, 2H), 6.98 (dd, 1H), 7.07-7.14 (m, 2H), 7.20 (dd, 1H), 7.29 (d, 1H), 7.37-7.44 (m, 3H), 7.58 (d, 1H), 7.77 (dd, 1H), 8.22 (d, 1H), 10.62 (s, 1H).

### Example 134

### N-[4-(3-Chlorophenoxy)-3-sulfamoylphenyl]-2-[2-chloro-5-(1,1,2,2-tetrafluoroethoxy)phenyl]acetamide

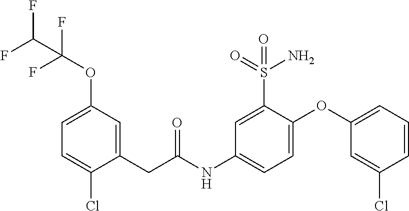 [SMILES: NS(=O)(=O)c1cc(NC(=O)Cc2cc(OC(F)(F)C(F)F)ccc2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and [2-chloro-5-(1,1,2,2-tetrafluoroethoxy)phenyl]acetic acid (49.2 mg, 0.172 mmol) were converted to N-[4-(3-chlorophenoxy)-3-sulfamoylphenyl]-2-[2-chloro-5-(1,1,2,2-tetrafluoroethoxy)phenyl]acetamide and were purified by preparative HPLC (Waters XBrigde C18 5μ 100×30 mm, acetonitrile/water+0.1% formic acid) (11 mg, 0.0194 mmol, 12% yield, 98% purity).

LC-MS (Method A): Rt=1.31 min

MS (ESIpos): m/z=567 (M+H)⁺

¹H-NMR (400 MHz, DMSO-d₆) δ [ppm]: 3.92 (s, 2H), 6.84 (tt, 1H), 6.98 (ddd, 1H), 7.08-7.13 (m, 2H), 7.20 (ddd, 1H), 7.27 (dd, 1H), 7.37-7.46 (m, 4H), 7.57 (d, 1H), 7.80 (dd, 1H), 8.22 (d, 1H), 10.58 (s, 1H).

### Example 135

### N-[4-(3-Chlorophenoxy)-3-sulfamoylphenyl]-2-[2-chloro-4-(trifluoromethyl)phenyl]acetamide

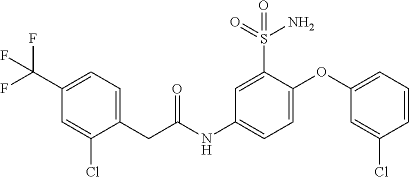 [SMILES: NS(=O)(=O)c1cc(NC(=O)Cc2ccc(C(F)(F)F)cc2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (100 mg, 0.22 mmol) and [2-chloro-4-(trifluoromethyl)phenyl]acetic acid (58.5 mg, 0.25 mmol) were converted to N-[4-(3-chlorophenoxy)-3-sulfamoylphenyl]-2-[2-chloro-4-(trifluoromethyl)phenyl]acetamide and were purified by preparative HPLC (Waters XBrigde C18 5μ 100×30 mm, acetonitrile/water+0.1% formic acid) (49 mg, 0.0944 mmol, 43% yield, 98% purity).

LC-MS (Method A): Rt=1.32 min

MS (ESIpos): m/z=519 (M+H)⁺

¹H-NMR (400 MHz, DMSO-d₆) δ [ppm]: 3.98 (s, 2H), 6.98 (dd, 1H), 7.07-7.13 (m, 2H), 7.20 (ddd, 1H), 7.36-7.45 (m, 3H), 7.67-7.75 (m, 2H), 7.80 (dd, 1H), 7.87-7.90 (m, 1H), 8.21 (d, 1H), 10.62 (s, 1H).

### Example 136

### 2-(2-Chlorophenyl)-N-(4-{[3-(methylsulfonyl)benzyl]oxy}-3-sulfamoylphenyl)acetamide

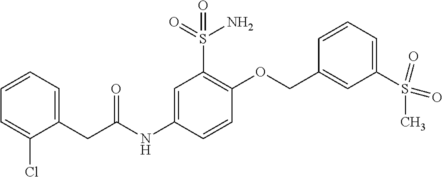 [SMILES: CS(=O)(=O)c1cccc(COc2ccc(NC(=O)Cc3ccccc3Cl)cc2S(N)(=O)=O)c1]

According to general procedure GP5, a solution of 2-(2-chlorophenyl)-N-(4-hydroxy-3-sulfamoylphenyl)acetamide (68.2 mg, 0.20 mmol) in dimethylformamide (3 mL) was cooled in an ice bath and treated with sodium hydride (10.5 mg, 0.24 mmol, 55% purity). After stirring for 20 min 1-(bromomethyl)-3-(methylsulfonyl)benzene (74.7 mg, 0.30 mmol) was added and the reaction mixture was allowed to warm up and was stirred at room temperature overnight. Water and ethyl acetate were added and the organic phase was removed, washed twice with water, was dried over sodium sulfate and concentrated in vacuo. The resulting residue was purified by preparative HPLC (Waters XBrigde C18 5μ 100×30 mm, acetonitrile/water+0.1% formic acid) to obtain 2-(2-chlorophenyl)-N-(4-{[3-(methylsulfonyl)benzyl]oxy}-3-sulfamoylphenyl)acetamide (55 mg, 0.108 mmol, 52% yield, 97% purity).

LC-MS (Method A): Rt=1.06 min

MS (ESIpos): m/z=509 (M+H)⁺

¹H-NMR (400 MHz, DMSO-d₆) δ [ppm]: 3.22 (s, 3H), 3.81 (s, 2H), 5.44 (s, 2H), 7.17 (s, 2H), 7.20 (d, 1H), 7.27-7.35 (m, 2H), 7.39-7.44 (m, 2H), 7.66 (t, 1H), 7.72 (dd, 1H), 7.83-7.89 (m, 2H), 8.07 (d, 1H), 8.11-8.13 (m, 1H), 10.33 (s, 1H).

### Example 137

### 2-(2-Chlorophenyl)-N-{4-[(2-fluorobenzyl)oxy]-3-sulfamoylphenyl}acetamide

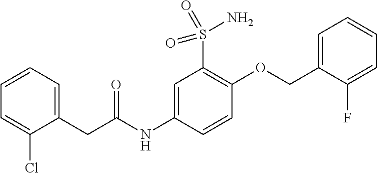 [SMILES: NS(=O)(=O)c1cc(NC(=O)Cc2ccccc2Cl)ccc1OCc1ccccc1F]

According to general procedure GP5, 2-(2-chlorophenyl)-N-(4-hydroxy-3-sulfamoylphenyl)acetamide (68.2 mg, 0.20 mmol) and 1-(bromomethyl)-2-fluorobenzene (56.7 mg, 0.30 mmol) were converted to 2-(2-chlorophenyl)-N-{4-[(2-fluorobenzyl)oxy]-3-sulfamoylphenyl}acetamide and was purified by preparative HPLC (Waters XBrigde C18 5μ 100×30 mm, acetonitrile/water+0.1% formic acid) (50 mg, 0.111 mmol, 56% yield, 99% purity).

LC-MS (Method A): Rt=1.14 min

MS (ESIpos): m/z=449 (M+H)⁺

¹H-NMR (400 MHz, DMSO-d₆) δ [ppm]: 3.81 (s, 2H), 5.37 (s, 2H), 7.04 (s, 2H), 7.17-7.35 (m, 5H), 7.36-7.47 (m, 3H), 7.63 (td, 1H), 7.74 (dd, 1H), 8.08 (d, 1H), 10.34 (s, 1H).

### Example 138

### 2-(2-Chlorophenyl)-N-{4-[(4-cyanobenzyl)oxy]-3-sulfamoylphenyl}acetamide

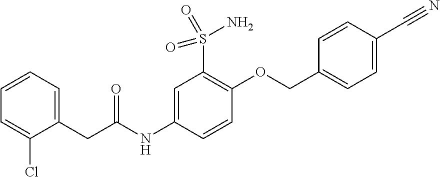 [SMILES: N#Cc1ccc(COc2ccc(NC(=O)Cc3ccccc3Cl)cc2S(N)(=O)=O)cc1]

According to general procedure GP5, 2-(2-chlorophenyl)-N-(4-hydroxy-3-sulfamoylphenyl)acetamide (51.1 mg, 0.150 mmol) and 4-(bromomethyl)benzonitrile (44.1 mg, 0.23 mmol) were converted to 2-(2-chlorophenyl)-N-{4-[(4-cyanobenzyl)oxy]-3-sulfamoylphenyl}acetamide and was purified by preparative HPLC (Waters XBrigde C18 5μ 100×30 mm, acetonitrile/water+0.2% aqueous ammonia (32%)) (30 mg, 0.0658 mmol, 44% yield, 99% purity).

LC-MS (Method B): Rt=1.04 min

MS (ESIneg): m/z=454 (M−H)⁺

¹H-NMR (400 MHz, DMSO-d₆) δ [ppm]: 3.79 (s, 2H), 5.41 (s, 2H), 7.10 (d, 1H), 7.15 (s, 2H), 7.25-7.33 (m, 2H), 7.37-7.45 (m, 2H), 7.65-7.71 (m, 3H), 7.82-7.86 (m, 2H), 8.05 (d, 1H), 10.30 (s, 1H).

### Example 139

### N-{4-[(3-Chlorobenzyl)oxy]-3-sulfamoylphenyl}-2-(2-chlorophenyl)acetamide

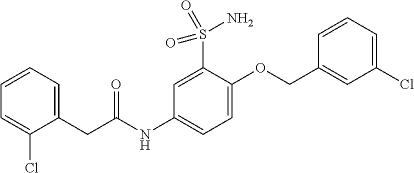 [SMILES: NS(=O)(=O)c1cc(NC(=O)Cc2ccccc2Cl)ccc1OCc1cccc(Cl)c1]

According to general procedure GP5, 2-(2-chlorophenyl)-N-(4-hydroxy-3-sulfamoylphenyl)acetamide (68.2 mg, 0.20 mmol) and 1-(bromomethyl)-3-chlorobenzene (61.6 mg, 0.30 mmol) were converted to N-{4-[(3-chlorobenzyl)oxy]-3-sulfamoylphenyl}-2-(2-chlorophenyl)acetamide and was purified by preparative HPLC (Waters XBrigde C18 5μ 100×30 mm, acetonitrile/water+0.1% formic acid) (40 mg, 0.0860 mmol, 43% yield, 97% purity).

LC-MS (Method A): Rt=1.27 min

MS (ESIpos): m/z=465 (M+H)⁺

¹H-NMR (400 MHz, METHANOL-d₄) δ [ppm]: 3.87 (s, 2H), 5.35 (s, 2H), 7.14 (d, 1H), 7.26-7.44 (m, 6H), 7.46-7.50 (m, 1H), 7.59 (s, 1H), 7.75 (dd, 1H), 8.09 (d, 1H).

### Example 140

### 2-(2-Chlorophenyl)-N-{4-[(3-methoxybenzyl)oxy]-3-sulfamoylphenyl}acetamide

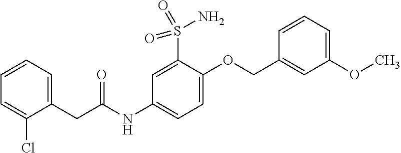 [SMILES: COc1cccc(COc2ccc(NC(=O)Cc3ccccc3Cl)cc2S(N)(=O)=O)c1]

According to general procedure GP5, 2-(2-chlorophenyl)-N-(4-hydroxy-3-sulfamoylphenyl)acetamide (68.2 mg, 0.20 mmol) and 1-(bromomethyl)-3-methoxybenzene (60.3 mg, 0.30 mmol) were converted to 2-(2-chlorophenyl)-N-{4-[(3-methoxybenzyl)oxy]-3-sulfamoylphenyl}acetamide and was purified by preparative HPLC (Waters XBrigde C18 5μ 100×30 mm, acetonitrile/water+0.1% formic acid) (25 mg, 0.0542 mmol, 27% yield, 97% purity).

LC-MS (Method A): Rt=1.20 min

MS (ESIpos): m/z=461 (M+H)⁺

¹H-NMR (400 MHz, METHANOL-d₄) δ [ppm]: 3.80 (s, 3H), 3.87 (s, 2H), 5.33 (s, 2H), 6.87 (dd, 1H), 7.08 (d, 1H), 7.15 (d, 1H), 7.17-7.20 (m, 1H), 7.26-7.33 (m, 3H), 7.37-7.46 (m, 2H), 7.74 (dd, 1H), 8.08 (d, 1H).

### Example 141

### N-[4-(Benzyloxy)-3-sulfamoylphenyl]-2-(2-chlorophenyl)acetamide

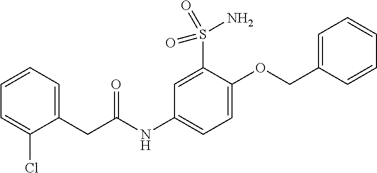 [SMILES: NS(=O)(=O)c1cc(NC(=O)Cc2ccccc2Cl)ccc1OCc1ccccc1]

According to general procedure GP5, 2-(2-chlorophenyl)-N-(4-hydroxy-3-sulfamoylphenyl)acetamide (102 mg, 0.30 mmol) and (bromomethyl)benzene (61.6 mg, 0.36 mmol) were converted to 2-(2-chlorophenyl)-N-{4-[(3-methoxybenzyl)oxy]-3-sulfamoylphenyl}acetamide and was purified by preparative HPLC (Chromatorex C-18 10 μm, 125×30 mm, acetonitrile/water+0.1% formic acid), followed by column chromatography on a Biotage Isolera system (silica gel, gradient dichloromethane/ethyl acetate) (20 mg, 0.0464 mmol, 15% yield, 99% purity).

LC-MS (Method B): Rt=1.12 min

MS (ESIpos): m/z=431 (M+H)⁺

¹H-NMR (400 MHz, METHANOL-d₄) δ [ppm]: 3.87 (s, 2H), 5.35 (s, 2H), 7.16 (d, 1H), 7.26-7.36 (m, 3H), 7.36-7.45 (m, 4H), 7.51-7.56 (m, 2H), 7.75 (dd, 1H), 8.08 (d, 1H).

### Example 142

### 2-(2-Chlorophenyl)-N-{4-[(3-cyanobenzyl)oxy]-3-sulfamoylphenyl}acetamide

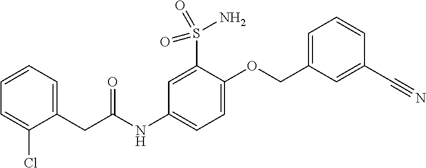 [SMILES: N#Cc1cccc(COc2ccc(NC(=O)Cc3ccccc3Cl)cc2S(N)(=O)=O)c1]

According to general procedure GP5, 2-(2-chlorophenyl)-N-(4-hydroxy-3-sulfamoylphenyl)acetamide (68.1 mg, 0.20 mmol) and 3-(bromomethyl)benzonitrile (58.8 mg, 0.30 mmol) were converted to 2-(2-chlorophenyl)-N-{4-[(3-cyanobenzyl)oxy]-3-sulfamoylphenyl}acetamide and was purified by preparative HPLC (Waters XBrigde C18 5μ 100×30 mm, acetonitrile/water+0.1% formic acid) (30 mg, 0.0658 mmol, 33% yield, 97% purity).

LC-MS (Method A): Rt=1.14 min

MS (ESIpos): m/z=456 (M+H)⁺

¹H NMR (400 MHz, DMSO-d₆) δ [ppm]: 3.81 (s, 2H), 5.38 (s, 2H), 7.16 (d, 1H), 7.19 (s, 2H), 7.27-7.35 (m, 2H), 7.38-7.48 (m, 2H), 7.60 (t, 1H), 7.69-7.75 (m, 1H), 7.78 (d, 1H), 7.85 (d, 1H), 7.98 (s, 1H), 8.04-8.09 (m, 1H), 10.33 (s, 1H).

### Example 143

### 2-(2-Chlorophenyl)-N-{4-[(4-fluorobenzyl)oxy]-3-sulfamoylphenyl}acetamide

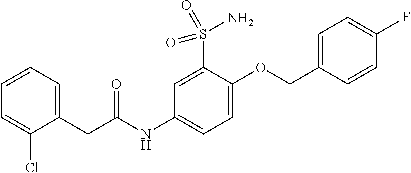 [SMILES: NS(=O)(=O)c1cc(NC(=O)Cc2ccccc2Cl)ccc1OCc1ccc(F)cc1]

According to general procedure GP5, 2-(2-chlorophenyl)-N-(4-hydroxy-3-sulfamoylphenyl)acetamide (68.1 mg, 0.20 mmol) and 1-(bromomethyl)-4-fluorobenzene (56.7 mg, 0.30 mmol) were converted to 2-(2-chlorophenyl)-N-{4-[(4-fluorobenzyl)oxy]-3-sulfamoylphenyl}acetamide and was purified by preparative HPLC (Waters XBrigde C18 5μ 100×30 mm, acetonitrile/water+0.1% formic acid) (43 mg, 0.0958 mmol, 48% yield, 99% purity).

LC-MS (Method A): Rt=1.14 min

MS (ESIpos): m/z=449 (M+H)⁺

¹H-NMR (400 MHz, DMSO-d₆) δ [ppm]: 3.79 (s, 2H), 5.29 (s, 2H), 7.06 (s, 2H), 7.11-7.22 (m, 3H), 7.25-7.33 (m, 2H), 7.37-7.46 (m, 2H), 7.52-7.58 (m, 2H), 7.69 (dd, 1H), 8.03 (d, 1H), 10.28 (s, 1H).

### Example 144

### N-{4-[(2-Chlorobenzyl)oxy]-3-sulfamoylphenyl}-2-(2-chlorophenyl)acetamide

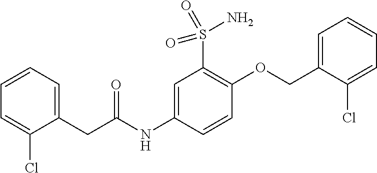 [SMILES: NS(=O)(=O)c1cc(NC(=O)Cc2ccccc2Cl)ccc1OCc1ccccc1Cl]

According to general procedure GP5, 2-(2-chlorophenyl)-N-(4-hydroxy-3-sulfamoylphenyl)acetamide (68.1 mg, 0.20 mmol) and 1-(bromomethyl)-2-chlorobenzene (61.6 mg, 0.30 mmol) were converted to N-{4-[(2-chlorobenzyl)oxy]-3-sulfamoylphenyl}-2-(2-chlorophenyl)acetamide and was purified by preparative HPLC (Waters XBrigde C18 5μ 100×30 mm, acetonitrile/water+0.1% formic acid) (50 mg, 0.107 mmol, 54% yield, 98% purity).

LC-MS (Method A): Rt=1.20 min

MS (ESIpos): m/z=465 (M+H)⁺

¹H-NMR (400 MHz, DMSO-d₆) δ [ppm]: 3.80 (s, 2H), 5.35 (s, 2H), 7.06 (s, 2H), 7.12 (d, 1H), 7.26-7.33 (m, 2H), 7.33-7.38 (m, 2H), 7.38-7.45 (m, 2H), 7.47-7.53 (m, 1H), 7.62-7.68 (m, 1H), 7.72 (dd, 1H), 8.09 (d, 1H), 10.32 (s, 1H).

### Example 145

### 2-(2-Chlorophenyl)-N-{4-[(2-cyanobenzyl)oxy]-3-sulfamoylphenyl}acetamide

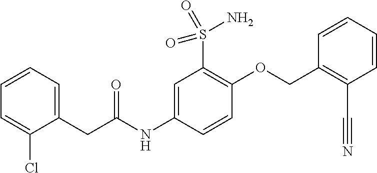 [SMILES: N#Cc1ccccc1COc1ccc(NC(=O)Cc2ccccc2Cl)cc1S(N)(=O)=O]

According to general procedure GP5, 2-(2-chlorophenyl)-N-(4-hydroxy-3-sulfamoylphenyl)acetamide (68.1 mg, 0.20 mmol) and 2-(bromomethyl)benzonitrile (58.8 mg, 0.30 mmol) were converted to 2-(2-chlorophenyl)-N-{4-[(2-cyanobenzyl)oxy]-3-sulfamoylphenyl}acetamide and was purified by preparative HPLC (Waters XBrigde C18 5μ 100×30 mm, acetonitrile/water+0.1% formic acid) (50 mg, 0.110 mmol, 55% yield, 99% purity).

LC-MS (Method A): Rt=1.09 min

MS (ESIpos): m/z=456 (M+H)⁺

¹H-NMR (400 MHz, DMSO-d₆) δ [ppm]: 3.80 (s, 2H), 5.46 (s, 2H), 7.06 (s, 2H), 7.20 (d, 1H), 7.26-7.34 (m, 2H), 7.38-7.45 (m, 2H), 7.53 (td, 1H), 7.70-7.82 (m, 3H), 7.90 (dd, 1H), 8.10 (d, 1H), 10.34 (s, 1H).

### Example 146

### N-[4-(Benzyloxy)-3-sulfamoylphenyl]-2-phenylacetamide

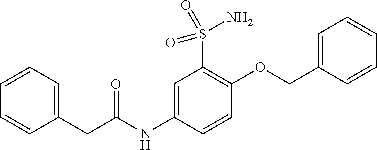 [SMILES: NS(=O)(=O)c1cc(NC(=O)Cc2ccccc2)ccc1OCc1ccccc1]

According to general procedure GP5, N-(4-hydroxy-3-sulfamoylphenyl)-2-phenylacetamide (153 mg, 0.50 mmol) and (bromomethyl)benzene (103 mg, 0.60 mmol) were converted to N-[4-(benzyloxy)-3-sulfamoylphenyl]-2-phenylacetamide and was purified by preparative HPLC (Chromatorex C-18 10 μm, 125×30 mm, acetonitrile/water+0.1% formic acid) (35 mg, 0.0883 mmol, 18% yield, 95% purity).

LC-MS (Method A): Rt=1.10 min

MS (ESIpos): m/z=397 (M+H)⁺

¹H-NMR (400 MHz, METHANOL-d₄) δ [ppm]: 3.65 (s, 2H), 5.32 (s, 2H), 7.13 (d, 1H), 7.28-7.40 (m, 8H), 7.49-7.54 (m, 2H), 7.72 (dd, 1H), 8.05 (d, 1H).

### Example 147

### 2-(2-Chlorophenyl)-N-(4-{[4-(methylsulfonyl)benzyl]oxy}-3-sulfamoylphenyl)acetamide

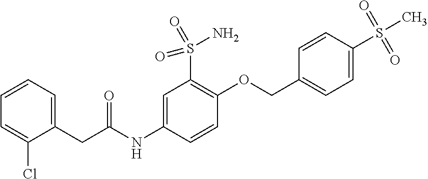 [SMILES: CS(=O)(=O)c1ccc(COc2ccc(NC(=O)Cc3ccccc3Cl)cc2S(N)(=O)=O)cc1]

According to general procedure GP5, 2-(2-chlorophenyl)-N-(4-hydroxy-3-sulfamoylphenyl)acetamide (68.1 mg, 0.20 mmol) and 1-(bromomethyl)-4-(methylsulfonyl)benzene (74.7 mg, 0.30 mmol) were converted to 2-(2-chlorophenyl)-N-(4-{[4-(methylsulfonyl)benzyl]oxy}-3-sulfamoylphenyl)acetamide and was purified by preparative HPLC (Waters XBrigde C18 5μ 100×30 mm, acetonitrile/water+0.1% formic acid) (52 mg, 0.103 mmol, 51% yield, 98% purity).

LC-MS (Method A): Rt=1.00 min

MS (ESIpos): m/z=509 (M+H)⁺

¹H-NMR (400 MHz, DMSO-d₆) δ [ppm]: 3.20 (s, 3H), 3.79 (s, 2H), 5.43 (s, 2H), 7.13 (d, 1H), 7.15 (s., 2H), 7.25-7.33 (m, 2H), 7.37-7.45 (m, 2H), 7.70 (dd, 1H), 7.75 (d, 2H), 7.91 (d, 2H), 8.06 (d, 1H), 10.30 (s, 1H).

### Example 148

### 2-(2-Chlorophenyl)-N-[4-(1-phenylethoxy)-3-sulfamoylphenyl]acetamide

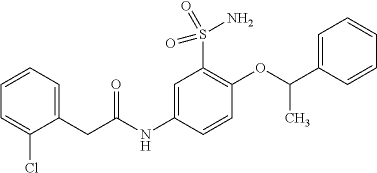 [SMILES: CC(Oc1ccc(NC(=O)Cc2ccccc2Cl)cc1S(N)(=O)=O)c1ccccc1]

According to general procedure GP5, 2-(2-chlorophenyl)-N-(4-hydroxy-3-sulfamoylphenyl)acetamide (68.1 mg, 0.20 mmol) and (1-bromoethyl)benzene (55.5 mg, 0.30 mmol) were converted to 2-(2-chlorophenyl)-N-[4-(1-phenylethoxy)-3-sulfamoylphenyl]acetamide and was purified by chiral preparative HPLC (instrument: Labomatic HD3000, AS-3000, Labcol Vario 4000 Plus, Knauer DAD 2600; column: Chiralpak IA 5μ 250×30 mm; eluent A: Hexan+0.1% Vol. Diethylamin (99%), eluent B: Ethanol; isocratic: 60% A+40% B; flow 40.0 mL/min; room temperature).

### Example 148A

1^{st} eluting Enantiomer: 32 mg, 0.0719 mmol, 36% yield, 99% purity, 99% ee

LC (Method E chiral): Rt=4.29 min

LC-MS (Method A): Rt=1.18 min

MS (ESIneg): m/z=443 (M−H)⁺

¹H-NMR (400 MHz, DMSO-d₆) δ [ppm]: 1.61 (d, 3H), 3.76 (s, 2H), 5.63 (q, 1H), 6.94 (d, 1H), 6.99 (s, 2H), 7.21-7.34 (m, 5H), 7.36-7.43 (m, 2H), 7.46-7.50 (m, 2H), 7.56 (dd, 1H), 8.02 (d, 1H), 10.22 (s, 1H).

### Example 148B

2^{nd} eluting Enantiomer: 35 mg, 0.0787 mmol, 39% yield, 98% purity, 99% ee

LC (Method E chiral): Rt=5.86 min

LC-MS (Method A): Rt=1.18 min

MS (ESIneg): m/z=443 (M−H)⁺

¹H-NMR (400 MHz, DMSO-d₆) δ [ppm]: 1.61 (d, 3H), 3.76 (s, 2H), 5.63 (q, 1H), 6.94 (d, 1H), 6.99 (s, 2H), 7.21-7.34 (m, 5H), 7.36-7.43 (m, 2H), 7.46-7.50 (m, 2H), 7.56 (dd, 1H), 8.02 (d, 1H), 10.22 (s, 1H).

### Example 149

### 2-(2-Chlorophenyl)-N-[4-(pyridin-3-ylmethoxy)-3-sulfamoylphenyl]acetamide

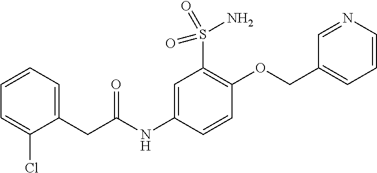 [SMILES: NS(=O)(=O)c1cc(NC(=O)Cc2ccccc2Cl)ccc1OCc1cccnc1]

According to general procedure GP5, 2-(2-chlorophenyl)-N-(4-hydroxy-3-sulfamoylphenyl)acetamide (102 mg, 0.30 mmol) and 3-(bromomethyl)pyridine hydrobromide (114 mg, 0.45 mmol)/N-ethyl-N-isopropylpropan-2-amine (116 mg, 0.90 mmol) were converted to 2-(2-chlorophenyl)-N-[4-(pyridin-3-ylmethoxy)-3-sulfamoylphenyl]acetamide (stirring overnight at room temperature was followed by stirring at 65° C. for 3 h) and was purified by preparative HPLC (Waters XBrigde C18 5μ 100×30 mm, acetonitrile/water+0.2% aqueous ammonia (32%)) (15 mg, 0.0347 mmol, 12% yield, 98% purity).

LC-MS (Method B): Rt=0.87 min

MS (ESIpos): m/z=432 (M+H)⁺

¹H-NMR (500 MHz, DMSO-d₆) δ [ppm]: 3.79 (s, 2H), 5.36 (s, 2H), 7.12 (s, 2H), 7.19 (d, 1H), 7.26-7.32 (m, 2H), 7.37-7.45 (m, 3H), 7.71 (dd, 1H), 7.92 (dt, 1H), 8.05 (d, 1H), 8.50 (dd, 1H), 8.71 (d, 1H), 10.30 (s, 1H).

### Example 150

### 2-(2-Chlorophenyl)-N-[4-(pyridin-2-ylmethoxy)-3-sulfamoylphenyl]acetamide

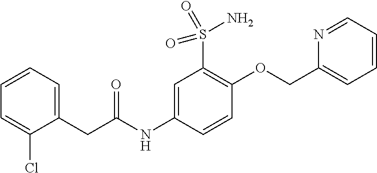 [SMILES: NS(=O)(=O)c1cc(NC(=O)Cc2ccccc2Cl)ccc1OCc1ccccn1]

According to general procedure GP5, 2-(2-chlorophenyl)-N-(4-hydroxy-3-sulfamoylphenyl)acetamide (102 mg, 0.30 mmol) and 2-(bromomethyl)pyridine hydrobromide (114 mg, 0.45 mmol)/N-ethyl-N-isopropylpropan-2-amine (116 mg, 0.90 mmol) were converted to 2-(2-chlorophenyl)-N-[4-(pyridin-2-ylmethoxy)-3-sulfamoylphenyl]acetamide (stirring overnight at room temperature was followed by stirring at 65° C. for 3 h) and was purified by preparative HPLC (Waters XBrigde C18 5μ 100×30 mm, acetonitrile/water+0.2% aqueous ammonia (32%)) (45 mg, 0.104 mmol, 35% yield, 99% purity).

LC-MS (Method B): Rt=0.94 min

MS (ESIpos): m/z=432 (M+H)⁺

¹H-NMR (500 MHz, DMSO-d₆) δ [ppm]: 3.80 (s, 2H), 5.35 (s, 2H), 7.20 (d, 1H), 7.27-7.33 (m, 2H), 7.33-7.38 (m, 3H), 7.39-7.45 (m, 2H), 7.57 (d, 1H), 7.73 (dd, 1H), 7.84 (td, 1H), 8.06 (d, 1H), 8.56-8.59 (m, 1H), 10.32 (s, 1H).

### Example 151

### 2-(2-Chlorophenyl)-N-[4-(pyridin-4-ylmethoxy)-3-sulfamoylphenyl]acetamide

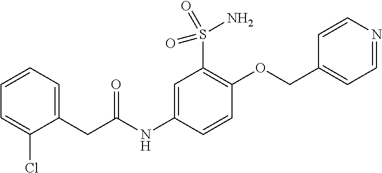 [SMILES: NS(=O)(=O)c1cc(NC(=O)Cc2ccccc2Cl)ccc1OCc1ccncc1]

According to general procedure GP5, 2-(2-chlorophenyl)-N-(4-hydroxy-3-sulfamoylphenyl)acetamide (102 mg, 0.30 mmol) and 4-(bromomethyl)pyridine (77.4 mg, 0.45 mmol) were converted to 2-(2-chlorophenyl)-N-[4-(pyridin-4-ylmethoxy)-3-sulfamoylphenyl]acetamide and was purified by preparative HPLC (Waters XBrigde C18 5μ 100×30 mm, acetonitrile/water+0.2% aqueous ammonia (32%)) (23 mg, 0.0535 mmol, 18% yield, 98% purity).

LC-MS (Method B): Rt=0.87 min

MS (ESIpos): m/z=432 (M+H)⁺

¹H-NMR (400 MHz, DMSO-d₆) δ [ppm]: 3.81 (s, 2H), 5.38 (s, 2H), 7.11 (d, 1H), 7.20 (s, 2H), 7.27-7.35 (m, 2H), 7.39-7.47 (m, 2H), 7.49 (d, 2H), 7.71 (dd, 1H), 8.08 (d, 1H), 8.54-8.58 (m, 2H), 10.33 (s, 1H).

### Example 152

### N-[4-(Pyridin-2-ylmethoxy)-3-sulfamoylphenyl]-2-[4-(trifluoromethyl)phenyl]acetamide

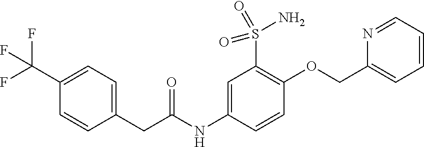 [SMILES: NS(=O)(=O)c1cc(NC(=O)Cc2ccc(C(F)(F)F)cc2)ccc1OCc1ccccn1]

According to general procedure GP5, N-(4-hydroxy-3-sulfamoylphenyl)-2-[4-(trifluoromethyl)phenyl]acetamide (112 mg, 0.30 mmol) and 2-(bromomethyl)pyridine hydrobromide (114 mg, 0.45 mmol)/N-ethyl-N-isopropylpropan-2-amine (116 mg, 0.90 mmol) were converted to N-[4-(pyridin-2-ylmethoxy)-3-sulfamoylphenyl]-2-[4-(trifluoromethyl)phenyl]acetamide and was purified by preparative HPLC (Waters XBrigde C18 5μ 100×30 mm, acetonitrile/water+0.2% aqueous ammonia (32%)) (45 mg, 0.104 mmol, 35% yield, 98% purity).

LC-MS (Method B): Rt=1.05 min

MS (ESIpos): m/z=466 (M+H)⁺

¹H-NMR (400 MHz, DMSO-d₆) δ [ppm]: 3.74 (s, 2H), 5.35 (s, 2H), 7.19 (d, 1H), 7.32-7.37 (m, 3H), 7.52-7.57 (m, 3H), 7.68 (d, 2H), 7.72 (d, 1H), 7.83 (td, 1H), 8.04 (d, 1H), 8.55-8.58 (m, 1H), 10.34 (s, 1H).

### Example 153

### 2-(2-Chlorophenyl)-N-[4-(pyrimidin-4-ylmethoxy)-3-sulfamoylphenyl]acetamide

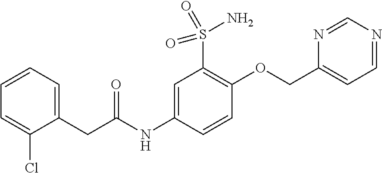 [SMILES: NS(=O)(=O)c1cc(NC(=O)Cc2ccccc2Cl)ccc1OCc1ccncn1]

According to general procedure GP5, 2-(2-chlorophenyl)-N-(4-hydroxy-3-sulfamoylphenyl)acetamide (102 mg, 0.30 mmol) and 4-(bromomethyl)pyrimidine hydrobromide (114 mg, 0.45 mmol)/N-ethyl-N-isopropylpropan-2-amine (116 mg, 0.90 mmol) were converted to 2-(2-chlorophenyl)-N-[4-(pyrimidin-4-ylmethoxy)-3-sulfamoylphenyl]acetamide (stirring overnight at room temperature was followed by stirring at 65° C. for 3 days) and was purified by preparative HPLC (Waters XBrigde C18 5μ 100×30 mm, acetonitrile/water+0.2% aqueous ammonia (32%)) (15 mg, 0.0347 mmol, 12% yield, 98% purity).

LC-MS (Method B): Rt=0.82 min

MS (ESIpos): m/z=433 (M+H)⁺

¹H-NMR (500 MHz, DMSO-d₆) δ [ppm]: 3.81 (s, 2H), 5.37 (s, 2H), 7.19 (d, 1H), 7.27 (s, 2H), 7.28-7.33 (m, 2H), 7.39-7.45 (m, 2H), 7.69 (dd, 1H), 7.74 (dd, 1H), 8.09 (d, 1H), 8.83 (d, 1H), 9.18 (d, 1H), 10.34 (s, 1H).

### Example 154

### 2-(2-Chlorophenyl)-N-[4-(pyrimidin-2-ylmethoxy)-3-sulfamoylphenyl]acetamide

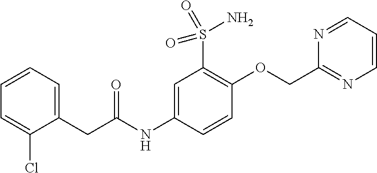 [SMILES: NS(=O)(=O)c1cc(NC(=O)Cc2ccccc2Cl)ccc1OCc1ncccn1]

According to general procedure GP5, 2-(2-chlorophenyl)-N-(4-hydroxy-3-sulfamoylphenyl)acetamide (102 mg, 0.30 mmol) and 2-(chloromethyl)pyrimidine hydrochloride (74.3 mg, 0.45 mmol)/N-ethyl-N-isopropylpropan-2-amine (116 mg, 0.90 mmol) were converted to 2-(2-chlorophenyl)-N-[4-(pyrimidin-2-ylmethoxy)-3-sulfamoylphenyl]acetamide (stirring overnight at room temperature was followed by stirring at 65° C. for 2 days and at 120° C. for another 2 days) and was purified by preparative HPLC (Waters XBrigde C18 5μ 100×30 mm, acetonitrile/water+0.2% aqueous ammonia (32%)) (19 mg, 0.0439 mmol, 15% yield, 97% purity).

LC-MS (Method B): Rt=0.88 min

MS (ESIpos): m/z=433 (M+H)⁺

¹H-NMR (500 MHz, DMSO-d₆) δ [ppm]: 3.81 (s, 2H), 5.46 (s, 2H), 7.27-7.34 (m, 3H), 7.40-7.45 (m, 2H), 7.50 (s, 2H), 7.52 (t, 1H), 7.74 (dd, 1H), 8.06 (d, 1H), 8.88 (d, 2H), 10.34 (s, 1H).

### Example 155

### 2-(2-Chlorophenyl)-N-[4-(2-phenylethoxy)-3-sulfamoylphenyl]acetamide

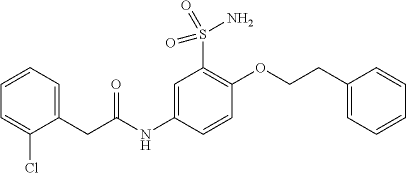 [SMILES: NS(=O)(=O)c1cc(NC(=O)Cc2ccccc2Cl)ccc1OCCc1ccccc1]

According to general procedure GP5, 2-(2-chlorophenyl)-N-(4-hydroxy-3-sulfamoylphenyl)acetamide (102 mg, 0.30 mmol) and (2-bromoethyl)benzene (83.3 mg, 0.45 mmol) were converted to 2-(2-chlorophenyl)-N-[4-(2-phenylethoxy)-3-sulfamoylphenyl]acetamide and was purified by preparative HPLC (Waters XBrigde C18 5μ 100×30 mm, acetonitrile/water+0.1% formic acid) (35 mg, 0.0787 mmol, 26% yield, 98% purity).

LC-MS (Method A): Rt=1.17 min

MS (ESIpos): m/z=445 (M+H)⁺

¹H-NMR (400 MHz, DMSO-d₆) δ [ppm]: 3.13 (t, 2H), 3.81 (s, 2H), 4.31 (t, 2H), 6.69 (s, 2H), 7.19-7.26 (m, 2H), 7.28-7.39 (m, 6H), 7.40-7.47 (m, 2H), 7.77 (dd, 1H), 8.03 (d, 1H), 10.32 (s, 1H).

### Example 156

### 2-(2-Chlorophenyl)-N-{4-[2-(3-chlorophenyl)ethoxy]-3-sulfamoylphenyl}acetamide

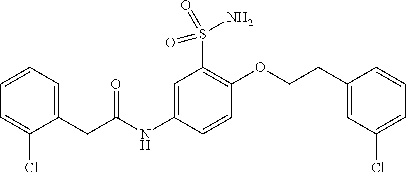 [SMILES: NS(=O)(=O)c1cc(NC(=O)Cc2ccccc2Cl)ccc1OCCc1cccc(Cl)c1]

According to general procedure GP5, 2-(2-chlorophenyl)-N-(4-hydroxy-3-sulfamoylphenyl)acetamide (102 mg, 0.30 mmol) and 1-(2-bromoethyl)-3-chlorobenzene (98.8 mg, 0.45 mmol) were converted to 2-(2-chlorophenyl)-N-{4-[2-(3-chlorophenyl)ethoxy]-3-sulfamoylphenyl}acetamide and was purified by preparative HPLC (Waters XBrigde C18 5μ 100×30 mm, acetonitrile/water+0.1% formic acid) (25 mg, 0.0521 mmol, 17% yield, 97% purity).

LC-MS (Method A): Rt=1.23 min

MS (ESIpos): m/z=479 (M+H)⁺

¹H-NMR (400 MHz, DMSO-d₆) δ [ppm]: 3.15 (t, 2H), 3.81 (s, 2H), 4.31 (t, 2H), 6.79 (s, 2H), 7.21 (d, 1H), 7.27-7.36 (m, 5H), 7.40-7.47 (m, 2H), 7.48-7.51 (m, 1H), 7.77 (dd, 1H), 8.03 (d, 1H), 10.31 (s, 1H).

### Example 157

### 2-(2-Chlorophenyl)-N-[4-(cyclobutylmethoxy)-3-sulfamoylphenyl]acetamide

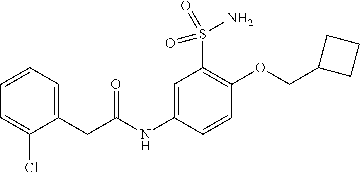 [SMILES: NS(=O)(=O)c1cc(NC(=O)Cc2ccccc2Cl)ccc1OCC1CCC1]

According to general procedure GP5, 2-(2-chlorophenyl)-N-(4-hydroxy-3-sulfamoylphenyl)acetamide (51.1 mg, 0.15 mmol) and (bromomethyl)cyclobutane (33.5 mg, 0.23 mmol) were converted to 2-(2-chlorophenyl)-N-[4-(cyclobutylmethoxy)-3-sulfamoylphenyl]acetamide and was purified by preparative HPLC (Waters XBrigde C18 5μ 100×30 mm, acetonitrile/water+0.2% aqueous ammonia (32%)) (10 mg, 0.0245 mmol, 16% yield, 98% purity).

LC-MS (Method B): Rt=1.13 min

MS (ESIpos): m/z=409 (M+H)⁺

¹H-NMR (400 MHz, DMSO-d₆) δ [ppm]: 1.78-1.93 (m, 4H), 1.97-2.08 (m, 2H), 2.73-2.87 (m, 1H), 3.80 (s, 2H), 4.08 (d, 2H), 6.77 (s, 2H), 7.16 (d, 1H), 7.26-7.34 (m, 2H), 7.38-7.45 (m, 2H), 7.75 (dd, 1H), 8.01 (d, 1H), 10.28 (s, 1H).

### Example 158

### 2-(2-Chlorophenyl)-N-[4-(oxetan-2-ylmethoxy)-3-sulfamoylphenyl]acetamide

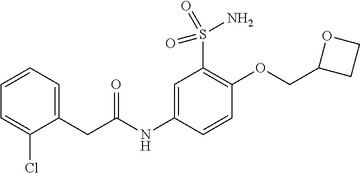 [SMILES: NS(=O)(=O)c1cc(NC(=O)Cc2ccccc2Cl)ccc1OCC1CCO1]

According to general procedure GP5, 2-(2-chlorophenyl)-N-(4-hydroxy-3-sulfamoylphenyl)acetamide (51.1 mg, 0.15 mmol) and 2-(bromomethyl)oxetane (40.0 mg, 0.23 mmol) were converted to 2-(2-chlorophenyl)-N-[4-(oxetan-2-ylmethoxy)-3-sulfamoylphenyl]acetamide and was purified by preparative HPLC (Waters XBrigde C18 5μ 100×30 mm, acetonitrile/water+0.2% aqueous ammonia (32%)) (6 mg, 0.0146 mmol, 10% yield, 95% purity).

LC-MS (Method B): Rt=0.90 min

MS (ESIpos): m/z=411 (M+H)⁺

¹H-NMR (400 MHz, DMSO-d₆) δ [ppm]: 2.61-2.71 (m, 2H), 3.81 (s, 2H), 4.18 (dd, 1H), 4.29 (dd, 1H), 4.48-4.60 (m, 2H), 5.05-5.14 (m, 1H), 6.90 (s, 2H), 7.25 (d, 1H), 7.27-7.34 (m, 2H), 7.39-7.46 (m, 2H), 7.77 (dd, 1H), 8.03 (d, 1H), 10.33 (s, 1H).

### Example 159

### 2-(2-Chlorophenyl)-N-[4-(oxetan-3-ylmethoxy)-3-sulfamoylphenyl]acetamide

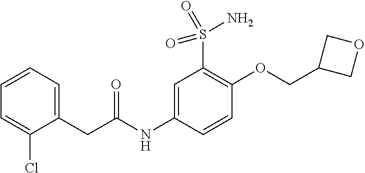 [SMILES: NS(=O)(=O)c1cc(NC(=O)Cc2ccccc2Cl)ccc1OCC1COC1]

According to general procedure GP5, 2-(2-chlorophenyl)-N-(4-hydroxy-3-sulfamoylphenyl)acetamide (51.1 mg, 0.15 mmol) and 3-(bromomethyl)oxetane (40.0 mg, 0.23 mmol) were converted to 2-(2-chlorophenyl)-N-[4-(oxetan-3-ylmethoxy)-3-sulfamoylphenyl]acetamide and was purified by preparative HPLC (Waters XBrigde C18 5μ 100×30 mm, acetonitrile/water+0.2% aqueous ammonia (32%)) (20 mg, 0.0487 mmol, 32% yield, 98% purity).

LC-MS (Method B): Rt=0.88 min

MS (ESIneg): m/z=409 (M−H)⁺

¹H-NMR (400 MHz, DMSO-d₆) δ [ppm]: 3.28-3.38 (m, 1H), 3.81 (s, 2H), 4.29 (d, 2H), 4.44 (t, 2H), 4.78 (dd, 2H), 6.99 (s, 2H), 7.21 (d, 1H), 7.26-7.34 (m, 2H), 7.39-7.46 (m, 2H), 7.77 (dd, 1H), 8.03 (d, 1H), 10.31 (s, 1H).

### Example 160

### 2-(2-Chlorophenyl)-N-[4-(cyclopentylmethoxy)-3-sulfamoylphenyl]acetamide

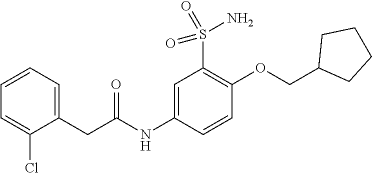 [SMILES: NS(=O)(=O)c1cc(NC(=O)Cc2ccccc2Cl)ccc1OCC1CCCC1]

According to general procedure GP5, 2-(2-chlorophenyl)-N-(4-hydroxy-3-sulfamoylphenyl)acetamide (51.1 mg, 0.15 mmol) and (bromomethyl)cyclopentane (36.7 mg, 0.23 mmol) were converted to 2-(2-chlorophenyl)-N-[4-(cyclopentylmethoxy)-3-sulfamoylphenyl]acetamide and was purified by preparative HPLC (Waters XBrigde C18 5μ 100×30 mm, acetonitrile/water+0.2% aqueous ammonia (32%)) (10 mg, 0.0236 mmol, 16% yield, 98% purity).

LC-MS (Method B): Rt=1.19 min

MS (ESIneg): m/z=421 (M−H)⁺

¹H-NMR (400 MHz, DMSO-d₆) δ [ppm]: 1.26-1.41 (m, 2H), 1.44-1.66 (m, 4H), 1.69-1.83 (m, 2H), 2.33-2.47 (m, 1H), 3.80 (s, 2H), 3.96 (d, 2H), 6.78 (s, 2H), 7.16 (d, 1H), 7.26-7.33 (m, 2H), 7.38-7.46 (m, 2H), 7.74 (dd, 1H), 8.01 (d, 1H), 10.27 (s, 1H).

### Example 161

### 2-(2-Chlorophenyl)-N-[3-sulfamoyl-4-(tetrahydrofuran-2-ylmethoxy)phenyl]acetamide

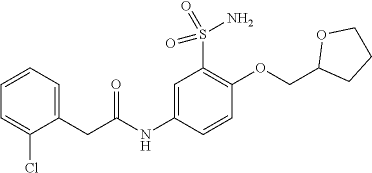 [SMILES: NS(=O)(=O)c1cc(NC(=O)Cc2ccccc2Cl)ccc1OCC1CCCO1]

According to general procedure GP5, 2-(2-chlorophenyl)-N-(4-hydroxy-3-sulfamoylphenyl)acetamide (51.1 mg, 0.15 mmol) and 2-(bromomethyl)tetrahydrofuran (37.1 mg, 0.23 mmol) were converted to 2-(2-chlorophenyl)-N-[3-sulfamoyl-4-(tetrahydrofuran-2-ylmethoxy)phenyl]acetamide and was purified by preparative HPLC (Waters XBrigde C18 5μ 100×30 mm, acetonitrile/water+0.2% aqueous ammonia (32%)) (3.5 mg, 0.00824 mmol, 5% yield, 98% purity).

LC-MS (Method B): Rt=0.99 min

MS (ESIneg): m/z=423 (M−H)⁺

¹H-NMR (400 MHz, DMSO-d₆) δ [ppm]: 1.63-1.74 (m, 1H), 1.77-1.94 (m, 2H), 1.94-2.04 (m, 1H), 3.65-3.72 (m, 1H), 3.74-3.79 (m, 1H), 3.80 (s, 2H), 3.92-3.96 (m, 1H), 4.19-4.27 (m, 2H), 6.87 (s, 2H), 7.21 (d, 1H), 7.26-7.33 (m, 2H), 7.39-7.46 (m, 2H), 7.76 (dd, 1H), 8.00 (d, 1H), 10.31 (s, 1H).

### Example 162

### 2-(2-Chlorophenyl)-N-[3-sulfamoyl-4-(tetrahydrofuran-3-ylmethoxy)phenyl]acetamide

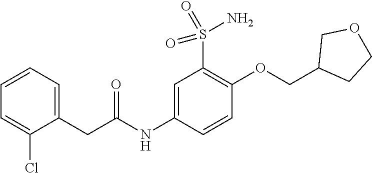 [SMILES: NS(=O)(=O)c1cc(NC(=O)Cc2ccccc2Cl)ccc1OCC1CCOC1]

According to general procedure GP5, 2-(2-chlorophenyl)-N-(4-hydroxy-3-sulfamoylphenyl)acetamide (51.1 mg, 0.15 mmol) and 3-(bromomethyl)tetrahydrofuran (37.1 mg, 0.23 mmol) were converted to 2-(2-chlorophenyl)-N-[3-sulfamoyl-4-(tetrahydrofuran-3-ylmethoxy)phenyl]acetamide and was purified by preparative HPLC (Waters XBrigde C18 5μ 100×30 mm, acetonitrile/water+0.2% aqueous ammonia (32%)) (10 mg, 0.0235 mmol, 16% yield, 98% purity).

LC-MS (Method B): Rt=0.94 min

MS (ESIneg): m/z=423 (M−H)⁺

¹H-NMR (400 MHz, DMSO-d₆) δ [ppm]: 1.67-1.78 (m, 1H), 1.98-2.11 (m, 1H), 2.70-2.81 (m, 1H), 3.56-3.75 (m, 3H), 3.80 (s, 2H), 3.82-3.89 (m, 1H), 4.00 (dd, 1H), 4.09 (dd, 1H), 6.94 (s, 2H), 7.16 (d, 1H), 7.26-7.33 (m, 2H), 7.37-7.45 (m, 2H), 7.76 (dd, 1H), 8.01 (d, 1H), 10.29 (s, 1H).

### Example 163

### 2-(2-Chloro-5-fluorophenyl)-N-[3-sulfamoyl-4-(tetrahydro-2H-pyran-4-ylmethoxy)phenyl]acetamide

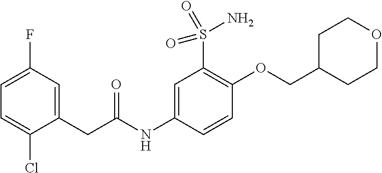 [SMILES: NS(=O)(=O)c1cc(NC(=O)Cc2cc(F)ccc2Cl)ccc1OCC1CCOCC1]

According to general procedure GP5, 2-(2-chloro-5-fluorophenyl)-N-(4-hydroxy-3-sulfamoylphenyl)acetamide (144 mg, 0.40 mmol) and 4-(bromomethyl)tetrahydro-2H-pyran (107 mg, 0.60 mmol) were converted to 2-(2-chloro-5-fluorophenyl)-N-[3-sulfamoyl-4-(tetrahydro-2H-pyran-4-ylmethoxy)phenyl]acetamide (stirring overnight at room temperature was followed by stirring at 50° C. for 6 h) and was purified by preparative HPLC (Chromatorex C-18 10 μm, 125×30 mm, acetonitrile/water+0.1% formic acid) (40 mg, 0.0875 mmol, 22% yield, 98% purity).

LC-MS (Method A): Rt=1.03 min

MS (ESIpos): m/z=457 (M+H)⁺

¹H-NMR (400 MHz, DMSO-d₆) δ [ppm]: 1.29 (ddd, 2H), 1.69-1.77 (m, 2H), 2.10-2.24 (m, 1H), 3.28-3.36 (m, 2H), 3.83 (s, 2H), 3.84-3.91 (m, 2H), 3.95 (d, 2H), 6.92 (s, 2H), 7.15-7.23 (m, 2H), 7.34 (dd, 1H), 7.50 (dd, 1H), 7.76 (dd, 1H), 8.02 (d, 1H), 10.33 (s, 1H).

### Example 164

### 2-(2-Chlorophenyl)-N-[3-sulfamoyl-4-(tetrahydro-2H-pyran-4-ylmethoxy)phenyl]acetamide

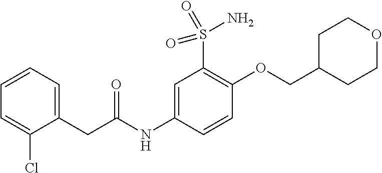 [SMILES: NS(=O)(=O)c1cc(NC(=O)Cc2ccccc2Cl)ccc1OCC1CCOCC1]

According to general procedure GP5, 2-(2-chlorophenyl)-N-(4-hydroxy-3-sulfamoylphenyl)acetamide (102 mg, 0.30 mmol) and 4-(bromomethyl)tetrahydro-2H-pyran (80.6 mg, 0.45 mmol) were converted to 2-(2-chlorophenyl)-N-[3-sulfamoyl-4-(tetrahydro-2H-pyran-4-ylmethoxy)phenyl]acetamide (stirring overnight at room temperature was followed by stirring at 65° C. for 25 h) and was purified by preparative HPLC (Waters XBrigde C18 5μ 100×30 mm, acetonitrile/water+0.2% aqueous ammonia (32%)) (23 mg, 0.0524 mmol, 17% yield, 80% purity).

LC-MS (Method B): Rt=0.94 min

MS (ESIneg): m/z=437 (M−H)⁺

¹H-NMR (500 MHz, DMSO-d₆) δ [ppm]: 1.22-1.34 (m, 2H), 1.70-1.77 (m, 2H), 2.10-2.23 (m, 1H), 3.27-3.35 (m, 2H), 3.80 (s, 2H), 3.83-3.88 (m, 2H), 3.93 (d, 2H), 6.88 (s, 2H), 7.16 (d, 1H), 7.27-7.33 (m, 2H), 7.42 (s, 2H), 7.73-7.77 (m, 1H), 8.01 (d, 1H), 10.28 (s, 1H).

### Example 165

### 2-(2-Chlorophenyl)-N-[3-sulfamoyl-4-(tetrahydro-2H-pyran-3-ylmethoxy)phenyl]acetamide

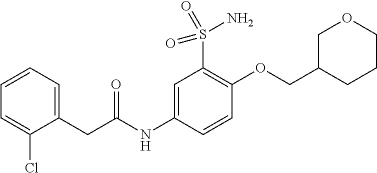 [SMILES: NS(=O)(=O)c1cc(NC(=O)Cc2ccccc2Cl)ccc1OCC1CCCOC1]

According to general procedure GP5, 2-(2-chlorophenyl)-N-(4-hydroxy-3-sulfamoylphenyl)acetamide (102 mg, 0.30 mmol) and 3-(bromomethyl)tetrahydro-2H-pyran (80.6 mg, 0.45 mmol) were converted to 2-(2-Chlorophenyl)-N-[3-sulfamoyl-4-(tetrahydro-2H-pyran-3-ylmethoxy)phenyl]acetamide (stirring overnight at room temperature was followed by stirring at 65° C. for 7 h) and was purified by preparative HPLC (Waters XBrigde C18 5μ 100×30 mm, acetonitrile/water+0.2% aqueous ammonia (32%)) (25 mg, 0.0570 mmol, 19% yield, 97% purity).

LC-MS (Method B): Rt=0.98 min

MS (ESIneg): m/z=437 (M−H)⁺

¹H-NMR (500 MHz, DMSO-d₆) δ [ppm]: 1.30-1.64 (m, 3H), 1.78-1.88 (m, 1H), 2.11-2.20 (m, 1H), 3.24-3.43 (m, 2H), 3.68-3.75 (m, 1H), 3.80 (s, 2H), 3.90-4.00 (m, 3H), 6.92 (s, 2H), 7.15 (d, 1H), 7.27-7.33 (m, 2H), 7.38-7.46 (m, 2H), 7.75 (dd, 1H), 8.01 (d, 1H), 10.28 (s, 1H).

### Example 166

### 2-(2-Chloro-6-fluorophenyl)-N-[3-sulfamoyl-4-(tetrahydro-2H-pyran-4-ylmethoxy)phenyl]acetamide

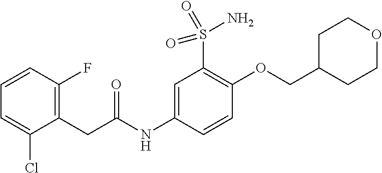 [SMILES: NS(=O)(=O)c1cc(NC(=O)Cc2c(F)cccc2Cl)ccc1OCC1CCOCC1]

According to general procedure GP5, 2-(2-chloro-6-fluorophenyl)-N-(4-hydroxy-3-sulfamoylphenyl)acetamide (53.8 mg, 0.15 mmol) and 4-(bromomethyl)tetrahydro-2H-pyran (40.3 mg, 0.23 mmol) were converted to 2-(2-chloro-6-fluorophenyl)-N-[3-sulfamoyl-4-(tetrahydro-2H-pyran-4-ylmethoxy)phenyl]acetamide (stirring over the weekend at room temperature) and was purified by preparative HPLC (Chromatorex C-18 10 μm, 125×30 mm, acetonitrile/water+0.1% formic acid) (3 mg, 0.00657 mmol, 4% yield, 99% purity).

LC-MS (Method A): Rt=1.02 min

MS (ESIpos): m/z=457 (M+H)⁺

¹H-NMR (400 MHz, DMSO-d₆) δ [ppm]: 1.22-1.37 (m, 2H), 1.68-1.79 (m, 2H), 2.09-2.24 (m, 1H), 3.26-3.36 (m, 2H), 3.87 (s, 4H), 3.94 (d, 2H), 6.92 (s, 2H), 7.17 (d, 1H), 7.25 (ddd, 1H), 7.33-7.42 (m, 2H), 7.74 (dd, 1H), 8.02 (d, 1H), 10.41 (s, 1H).

### Example 167

### 2-(2-Chloro-3-fluorophenyl)-N-[3-sulfamoyl-4-(tetrahydro-2H-pyran-4-ylmethoxy)phenyl]acetamide

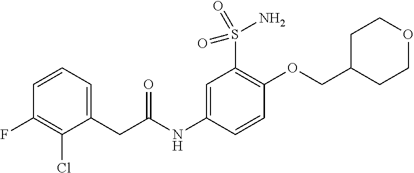 [SMILES: NS(=O)(=O)c1cc(NC(=O)Cc2cccc(F)c2Cl)ccc1OCC1CCOCC1]

According to general procedure GP5, 2-(2-chloro-3-fluorophenyl)-N-(4-hydroxy-3-sulfamoylphenyl)acetamide (233 mg, 0.65 mmol) and 4-(bromomethyl)tetrahydro-2H-pyran (175 mg, 0.98 mmol) were converted to 2-(2-chloro-3-fluorophenyl)-N-[3-sulfamoyl-4-(tetrahydro-2H-pyran-4-ylmethoxy)phenyl]acetamide (stirring over the weekend at room temperature) and was purified by preparative HPLC (Chromatorex C-18 10 μm, 125×30 mm, acetonitrile/water+0.1% formic acid) (35 mg, 0.0766 mmol, 12% yield, 99% purity).

LC-MS (Method B): Rt=1.00 min

MS (ESIpos): m/z=457 (M+H)⁺

¹H-NMR (400 MHz, DMSO-d₆) δ [ppm]: 1.22-1.35 (m, 2H), 1.67-1.78 (m, 2H), 2.10-2.24 (m, 1H), 3.28-3.36 (m, 2H), 3.88 (m, 4H), 3.94 (d, 2H), 6.92 (s, 2H), 7.18 (d, 1H), 7.26-7.41 (m, 3H), 7.76 (dd, 1H), 8.03 (d, 1H), 10.36 (s, 1H).

### Example 168

### 2-(2-chlorophenyl)-N-{5-sulfamoyl-6-[3-(trifluoromethyl)phenoxy]pyridin-3-yl}acetamide

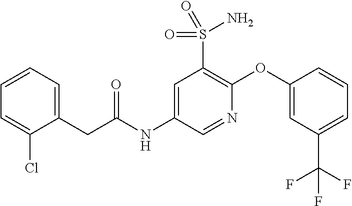 [SMILES: NS(=O)(=O)c1cc(NC(=O)Cc2ccccc2Cl)cnc1Oc1cccc(C(F)(F)F)c1]

Crude 5-amino-2-[3-(trifluoromethyl)phenoxy]pyridine-3-sulfonamide (150 mg) was dissolved in dimethylformamide (3 mL) followed by the addition of (2-chlorophenyl)acetic acid (84.5 mg, 0.495 mmol), N,N-diisopropylethylamine (291 mg, 2.25 mmol) and HATU (274 mg, 0.720 mmol). The reaction mixture was stirred at 50° C. for 4 h. After cooling to room temperature ethyl acetate and water were added to the reaction mixture and phases were separated. The aqueous phase was extracted again with ethyl acetate and the combined organic phases were dried over sodium sulfate and the solvent was removed under reduced pressure. Column chromatography on a Biotage Isolera system (silica gel, gradient n-hexane/ethyl acetate), followed by preparative HPLC (Waters XBrigde C18 5μ 100×30 mm, acetonitrile/water+0.2% aqueous ammonia (32%)) led to 2-(2-chlorophenyl)-N-{5-sulfamoyl-6-[3-(trifluoromethyl)phenoxy]pyridin-3-yl}acetamide (4.6 mg, 0.00947 mmol, 3% yield over 4 steps, 98% purity).

LC-MS (Method B): Rt=1.06 min

MS (ESIpos): m/z=486 (M+H)⁺

¹H-NMR (500 MHz, DICHLOROMETHANE-d₂) δ [ppm]: 3.90 (s, 2H), 5.34 (s, 2H), 7.31-7.37 (m, 2H), 7.40-7.45 (m, 2H), 7.46-7.51 (m, 2H), 7.54-7.64 (m, 2H), 8.38-8.46 (m, 2H).

### Example 169

### 2-Phenyl-N-{5-sulfamoyl-6-[3-(trifluoromethyl)phenoxy]pyridin-3-yl}acetamide

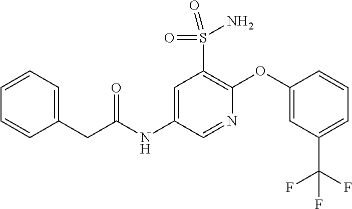 [SMILES: NS(=O)(=O)c1cc(NC(=O)Cc2ccccc2)cnc1Oc1cccc(C(F)(F)F)c1]

Crude 5-amino-2-[3-(trifluoromethyl)phenoxy]pyridine-3-sulfonamide (150 mg) was dissolved in dimethylformamide (3 mL) followed by the addition of phenylacetic acid (67.4 mg, 0.495 mmol), N,N-diisopropylethylamine (291 mg, 2.25 mmol) and HATU (274 mg, 0.720 mmol). The reaction mixture was stirred at 50° C. for 4 h. After cooling to room temperature ethyl acetate and water were added to the reaction mixture and phases were separated. The aqueous phase was extracted again with ethyl acetate and the combined organic phases were dried over sodium sulfate and the solvent was removed under reduced pressure. Column chromatography on a Biotage Isolera system (silica gel, gradient n-hexane/ethyl acetate), followed by preparative HPLC (Waters XBrigde C18 5μ 100×30 mm, acetonitrile/water+0.2% aqueous ammonia (32%)) led to 2-phenyl-N-{5-sulfamoyl-6-[3-(trifluoromethyl)phenoxy]pyridin-3-yl}acetamide (5.8 mg, 0.0128 mmol, 4% yield over 4 steps, 98% purity).

LC-MS (Method B): Rt=0.99 min

MS (ESIpos): m/z=452 (M+H)⁺

¹H-NMR (400 MHz, DICHLOROMETHANE-d₂) δ [ppm]: 3.79 (s, 2H), 5.36 (s, 2H), 7.30-7.48 (m, 6H), 7.50 (s, 1H), 7.56-7.66 (m, 2H), 8.40-8.46 (m, 2H).

### Example 170

### N-[4-(3-Chlorophenoxy)-3-fluoro-5-sulfamoylphenyl]-2-phenylacetamide

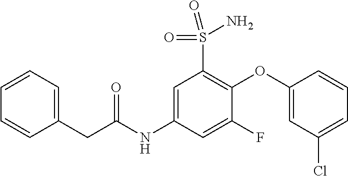 [SMILES: NS(=O)(=O)c1cc(NC(=O)Cc2ccccc2)cc(F)c1Oc1cccc(Cl)c1]

5-Amino-2-(3-chlorophenoxy)-3-fluorobenzenesulfonamide (166 mg (70%), 0.366 mmol) was dissolved in dimethylformamide (0.75 mL) followed by the addition of phenyl acetic acid (54.9 mg, 0.404 mmol), N,N-diisopropylethylamine (237 mg, 1.83 mmol) and HATU (223 mg, 0.587 mmol). The reaction mixture was stirred overnight at room temperature. Then, ethyl acetate and water were added to the reaction mixture and phases were separated. The organic phase was dried over sodium sulfate and the solvent was removed under reduced pressure. Preparative HPLC (Chromatorex C-18 10 μm, 125×30 mm, acetonitrile/water+0.1% formic acid) led to N-[4-(3-chlorophenoxy)-3-fluoro-5-sulfamoylphenyl]-2-phenylacetamide (4.0 mg, 0.00920 mmol, 3% yield, 85% purity).

LC-MS (Method A): Rt=1.33 min

MS (ESIpos): m/z=435 (M+H)⁺

¹H-NMR (400 MHz, DICHLOROMETHANE-d₂) δ [ppm]: 3.69 (s, 2H), 5.02 (s, 2H), 6.97 (dt, 1H), 7.03-7.45 (m, 10H), 7.80 (dd, 1H).

### Example 171

### N-[4-(3-Chlorophenoxy)-3-fluoro-5-sulfamoylphenyl]-2-(2-methylphenyl)acetamide

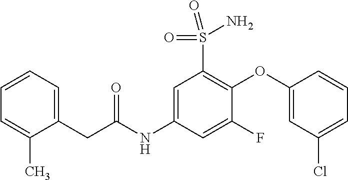 [SMILES: Cc1ccccc1CC(=O)Nc1cc(F)c(Oc2cccc(Cl)c2)c(S(N)(=O)=O)c1]

5-Amino-2-(3-chlorophenoxy)-3-fluorobenzenesulfonamide (134 mg (70%), 0.297 mmol) was dissolved in dimethylformamide (0.75 mL) followed by the addition of (2-methylphenyl)acetic acid (49.0 mg, 0.326 mmol), N,N-diisopropylethylamine (192 mg, 1.48 mmol) and HATU (181 mg, 0.475 mmol). The reaction mixture was stirred overnight at room temperature. Then, ethyl acetate and water were added to the reaction mixture and phases were separated. The organic phase was dried over sodium sulfate and the solvent was removed under reduced pressure. Preparative HPLC (Chromatorex C-18 10 μm, 125×30 mm, acetonitrile/water+0.1% formic acid) led to N-[4-(3-chlorophenoxy)-3-fluoro-5-sulfamoylphenyl]-2-(2-methylphenyl)acetamide (14.5 mg, 0.0323 mmol, 11% yield, 95% purity).

LC-MS (Method A): Rt=1.35 min

MS (ESIpos): m/z=449 (M+H)⁺

¹H-NMR (400 MHz, DICHLOROMETHANE-d₂) δ [ppm]: 3.69 (s, 2H), 5.03 (s, 2H), 6.95-7.06 (m, 2H), 7.11-7.15 (m, 2H), 7.21-7.32 (m, 6H), 7.78 (dd, 1H).

### Example 172

### N-[4-(3-Chlorophenoxy)-3-fluoro-5-sulfamoylphenyl]-2-(3-methylphenyl)acetamide

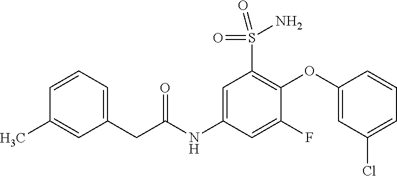 [SMILES: Cc1cccc(CC(=O)Nc2cc(F)c(Oc3cccc(Cl)c3)c(S(N)(=O)=O)c2)c1]

5-Amino-2-(3-chlorophenoxy)-3-fluorobenzenesulfonamide (160 mg (70%), 0.353 mmol) was dissolved in dimethylformamide (0.75 mL) followed by the addition of (3-methylphenyl)acetic acid (58.4 mg, 0.389 mmol), N,N-diisopropylethylamine (229 mg, 1.77 mmol) and HATU (215 mg, 0.566 mmol). The reaction mixture was stirred overnight at room temperature. Then, ethyl acetate and water were added to the reaction mixture and phases were separated. The organic phase was dried over sodium sulfate and the solvent was removed under reduced pressure. Preparative HPLC (Chromatorex C-18 10 μm, 125×30 mm, acetonitrile/water+0.1% formic acid) led to N-[4-(3-chlorophenoxy)-3-fluoro-5-sulfamoylphenyl]-2-(3-methylphenyl)acetamide (11.8 mg, 0.0263 mmol, 7% yield, 95% purity).

LC-MS (Method B): Rt=1.39 min

MS (ESIpos): m/z=449 (M+H)⁺

¹H-NMR (400 MHz, DICHLOROMETHANE-d₂) δ [ppm]: 1.59 (s, 3H), 3.63 (s, 2H), 5.02 (s, 2H), 6.97 (dt, 1H), 7.03-7.22 (m, 6H), 7.24-7.32 (m, 3H), 7.80 (dd, 1H).

### Example 173

### 2-(2-Chlorophenyl)-N-{4-[3-(3-oxomorpholin-4-yl)phenoxy]-3-sulfamoylphenyl}acetamide

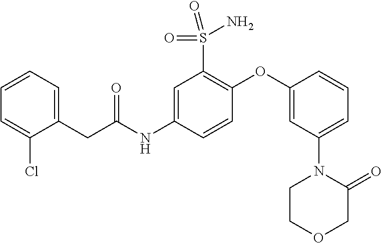 [SMILES: NS(=O)(=O)c1cc(NC(=O)Cc2ccccc2Cl)ccc1Oc1cccc(N2CCOCC2=O)c1]

N-{4-(3-Bromophenoxy)-3-[(2,4-dimethoxybenzyl)sulfamoyl]phenyl}-2-(2-chlorophenyl)acetamide (129 mg, 0.20 mmol), cesium carbonate (97.7 mg, 0.30 mmol), morpholin-3-one (24.3 mg, 0.24 mmol), Xanthphos (2.3 mg, 0.004 mmol) and tris(dibenzylideneacetone)dipalladium(0) (9.2 mg, 0.01 mmol) were dissolved in dioxane (5 mL), flushed with argon and stirred in a sealed vessel at 105° C. for 2.5 h. Then, the same amount of Xanthphos and tris(dibenzylideneacetone)dipalladium(0) was added and stirring at 105° C. was continued overnight. After cooling to room temperature the solvents were removed under reduced pressure and the residue was treated with water and dichloromethane. The organic phase was dried over sodium sulfate and concentrated, followed by chromatography over a Biotage Isolera system (silica gel, gradient dichloromethane/ethyl acetate). Then, the residue was redissolved in dichloromethane (10 mL), treated with trifluoroacetic acid (0.25 mL) and stirred overnight at room temperature. All volatile components were removed under reduced pressure followed by preparative HPLC (Chromatorex C-18 10 μm, 125×30 mm, acetonitrile/water+0.1% formic acid) leading to 2-(2-chlorophenyl)-N-{4-[3-(3-oxomorpholin-4-yl)phenoxy]-3-sulfamoylphenyl}acetamide (5.2 mg, 0.0102 mmol, 5% yield, 98% purity).

LC-MS (Method A): Rt=0.97 min

MS (ESIpos): m/z=516 (M+H)⁺

¹H-NMR (400 MHz, METHANOL-d₄) δ [ppm]: 3.78-3.83 (m, 2H), 3.90 (s, 2H), 4.01-4.07 (m, 2H), 4.29 (s, 2H), 7.04 (d, 1H), 7.08 (ddd, 1H), 7.18 (t, 1H), 7.21 (ddd, 1H), 7.27-7.35 (m, 2H), 7.39-7.51 (m, 3H), 7.79 (dd, 1H), 8.22 (d, 1H).

### Example 174

### 2-(2-Chlorophenyl)-N-{4-[4-(3-oxomorpholin-4-yl)phenoxy]-3-sulfamoylphenyl}acetamide

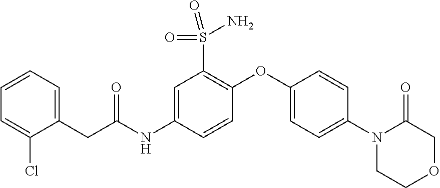 [SMILES: NS(=O)(=O)c1cc(NC(=O)Cc2ccccc2Cl)ccc1Oc1ccc(N2CCOCC2=O)cc1]

N-{4-(4-Bromophenoxy)-3-[(2,4-dimethoxybenzyl)sulfamoyl]phenyl}-2-(2-chlorophenyl)acetamide (129 mg, 0.20 mmol), cesium carbonate (97.7 mg, 0.30 mmol), morpholin-3-one (24.3 mg, 0.24 mmol), Xanthphos (2.3 mg, 0.004 mmol) and tris(dibenzylideneacetone)dipalladium(0) (9.2 mg, 0.01 mmol) were dissolved in dioxane (5 mL), flushed with argon and stirred in a sealed vessel at 105° C. for 2.5 h. Then, the same amount of Xanthphos and tris(dibenzylideneacetone)dipalladium(0) was added and stirring at 105° C. was continued overnight. After cooling to room temperature the solvents were removed under reduced pressure and the residue was treated with water and dichloromethane. The organic phase was dried over sodium sulfate and concentrated, followed by chromatography over a Biotage Isolera system (silica gel, dichloromethane/ethyl acetate gradient). Then, the residue was redissolved in dichloromethane (10 mL), treated with trifluoroacetic acid (0.25 mL) and stirred overnight at room temperature. All volatile components were removed under reduced pressure followed by preparative HPLC (Chromatorex C-18 10 μm, 125×30 mm, acetonitrile/water+0.1% formic acid) leading to 2-(2-chlorophenyl)-N-{4-[4-(3-oxomorpholin-4-yl)phenoxy]-3-sulfamoylphenyl}acetamide (1.3 mg, 0.00252 mmol, 1% yield, 98% purity).

LC-MS (Method A): Rt=0.95 min

MS (ESIpos): m/z=516 (M+H)⁺

¹H-NMR (400 MHz, METHANOL-d₄) δ [ppm]: 3.77-3.82 (m, 2H), 3.90 (s, 2H), 4.04-4.09 (m, 2H), 4.30 (s, 2H), 7.01 (d, 1H), 7.16-7.21 (m, 2H), 7.27-7.34 (m, 2H), 7.37-7.45 (m, 4H), 7.79 (dd, 1H), 8.22 (d, 1H).

### Example 175

### 2-(2-Chlorophenyl)-N-{4-[4-(2-oxopiperidin-1-yl)phenoxy]-3-sulfamoylphenyl}acetamide

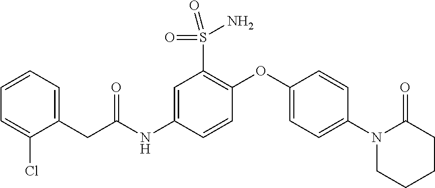 [SMILES: NS(=O)(=O)c1cc(NC(=O)Cc2ccccc2Cl)ccc1Oc1ccc(N2CCCCC2=O)cc1]

N-{4-(4-Bromophenoxy)-3-[(2,4-dimethoxybenzyl)sulfamoyl]phenyl}-2-(2-chlorophenyl)acetamide (129 mg, 0.20 mmol), cesium carbonate (97.7 mg, 0.30 mmol), piperidin-2-one (23.8 mg, 0.24 mmol), Xanthphos (2.3 mg, 0.004 mmol) and tris(dibenzylideneacetone)dipalladium(0) (9.2 mg, 0.01 mmol) were dissolved in dioxane (5 mL), flushed with argon and stirred in a sealed vessel at 105° C. for 2.5 h. Then, the same amount of Xanthphos and tris(dibenzylideneacetone)dipalladium(0) was added and stirring at 105° C. was continued overnight. After cooling to room temperature the solvents were removed under reduced pressure and the residue was treated with water and dichloromethane. The organic phase was dried over sodium sulfate and concentrated, followed by chromatography over a Biotage Isolera system (silica gel, gradient dichloromethane/ethyl acetate). Then, the residue was redissolved in dichloromethane (10 mL), treated with trifluoroacetic acid (0.25 mL) and stirred overnight at room temperature. All volatile components were removed under reduced pressure followed by preparative HPLC (Chromatorex C-18 10 μm, 125×30 mm, acetonitrile/water+0.1% formic acid) leading to 2-(2-chlorophenyl)-N-{4-[4-(2-oxopiperidin-1-yl)phenoxy]-3-sulfamoylphenyl}acetamide (1.0 mg, 0.00195 mmol, 1% yield, 95% purity).

LC-MS (Method A): Rt=1.02 min

MS (ESIpos): m/z=514 (M+H)⁺

¹H-NMR (400 MHz, METHANOL-d₄) δ [ppm]: 1.93-2.05 (m, 4H), 2.50-2.58 (m, 2H), 3.66-3.72 (m, 2H), 3.90 (s, 2H), 7.01 (s, 1H), 7.14-7.20 (m, 2H), 7.27-7.35 (m, 4H), 7.39-7.46 (m, 2H), 7.78 (dd, 1H), 8.22 (d, 1H).

### Example 176

### 2-(2-Chlorophenyl)-N-{4-[3-(2-oxopiperidin-1-yl)phenoxy]-3-sulfamoylphenyl}acetamide

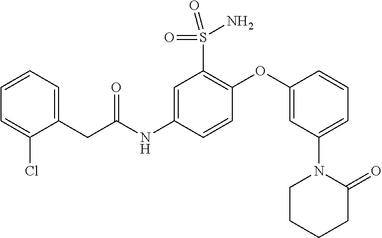 [SMILES: NS(=O)(=O)c1cc(NC(=O)Cc2ccccc2Cl)ccc1Oc1cccc(N2CCCCC2=O)c1]

N-{4-(3-Bromophenoxy)-3-[(2,4-dimethoxybenzyl)sulfamoyl]phenyl}-2-(2-chlorophenyl)acetamide (129 mg, 0.20 mmol), cesium carbonate (97.7 mg, 0.30 mmol), piperidin-2-one (23.8 mg, 0.24 mmol), Xanthphos (2.3 mg, 0.004 mmol) and tris(dibenzylideneacetone)dipalladium(0) (9.2 mg, 0.01 mmol) were dissolved in dioxane (5 mL), flushed with argon and stirred in a sealed vessel at 105° C. for 2.5 h. Then, the same amount of Xanthphos and tris(dibenzylideneacetone)dipalladium(0) was added and stirring at 105° C. was continued overnight. After cooling to room temperature the solvents were removed under reduced pressure and the residue was treated with water and dichloromethane. The organic phase was dried over sodium sulfate and concentrated, followed by chromatography over a Biotage Isolera system (silica gel, gradient dichloromethane/ethyl acetate). Then, the residue was redissolved in dichloromethane (10 mL), treated with trifluoroacetic acid (0.25 mL) and stirred overnight at room temperature. All volatile components were removed under reduced pressure followed by preparative HPLC (Chromatorex C-18 10 μm, 125×30 mm, acetonitrile/water+0.1% formic acid) leading to 2-(2-chlorophenyl)-N-{4-[3-(2-oxopiperidin-1-yl)phenoxy]-3-sulfamoylphenyl}acetamide (0.75 mg, 0.00146 mmol, 1% yield, 98% purity).

LC-MS (Method A): Rt=1.03 min

MS (ESIpos): m/z=514 (M+H)⁺

¹H-NMR (400 MHz, METHANOL-d₄) δ [ppm]: 1.90-2.03 (m, 4H), 2.49-2.55 (m, 2H), 3.66-3.72 (m, 2H), 3.90 (s, 2H), 7.03-7.08 (m, 3H), 7.11 (ddd, 1H), 7.27-7.35 (m, 2H), 7.39-7.48 (m, 3H), 7.75-7.81 (dd, 1H), 8.22 (d, 1H).

### Example 177

### 2-(2-Chlorophenyl)-N-{4-[3-(prop-1-en-2-yl)phenoxy]-3-sulfamoylphenyl}acetamide

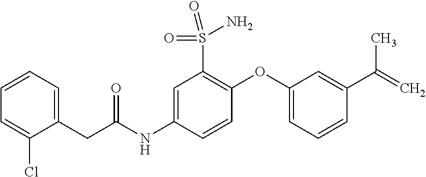 [SMILES: C=C(C)c1cccc(Oc2ccc(NC(=O)Cc3ccccc3Cl)cc2S(N)(=O)=O)c1]

2-(2-Chlorophenyl)-N-{3-[(2,4-dimethoxybenzyl)sulfamoyl]-4-[3-(2-hydroxypropan-2-yl)phenoxy]phenyl}acetamide (40.6 mg, 0.07 mmol) was dissolved in dichloromethane (10 mL) and treated with trifluoroacetic acid (0.2 mL). After stirring for 3 h at room temperature all volatile components were removed in vacuo and the residue was purified by preparative HPLC (Chromatorex C-18 10 μm, 125×30 mm, acetonitrile/water+0.1% formic acid) to give 2-(2-chlorophenyl)-N-{4-[3-(prop-1-en-2-yl)phenoxy]-3-sulfamoylphenyl}acetamide (5.8 mg, 0.0127 mmol, 18% yield, 98% purity).

LC-MS (Method A): Rt=1.24 min

MS (ESIpos): m/z=457 (M+H)⁺

¹H-NMR (400 MHz, DMSO-d₆) δ [ppm]: 2.10 (s, 3H), 3.84 (s, 2H), 5.12-5.16 (m, 1H), 5.42-5.45 (m, 1H), 6.94 (ddd, 1H), 6.98 (d, 1H), 7.23 (t, 1H), 7.28-7.49 (m, 8H), 7.75 (dd, 1H), 8.21 (d, 1H), 10.48 (s, 1H).

### Example 178

### 2-(2-Chlorophenyl)-N-{4-[2-(prop-1-en-2-yl)phenoxy]-3-sulfamoylphenyl}acetamide

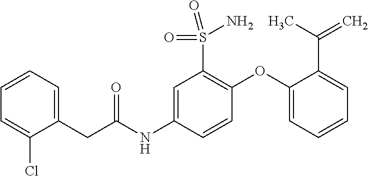 [SMILES: C=C(C)c1ccccc1Oc1ccc(NC(=O)Cc2ccccc2Cl)cc1S(N)(=O)=O]

2-(2-Chlorophenyl)-N-{3-[(2,4-dimethoxybenzyl)sulfamoyl]-4-[2-(2-hydroxypropan-2-yl)phenoxy]phenyl}acetamide (31.3 mg, 0.05 mmol) was dissolved in dichloromethane (10 mL) and treated with trifluoroacetic acid (0.2 mL). After stirring for 3 h at room temperature all volatile components were removed in vacuo and the residue was purified by preparative HPLC (Chromatorex C-18 10 μm, 125×30 mm, acetonitrile/water+0.1% formic acid) to give 2-(2-chlorophenyl)-N-{4-[2-(prop-1-en-2-yl)phenoxy]-3-sulfamoylphenyl}acetamide (5.0 mg, 0.0109 mmol, 22% yield, 98% purity).

LC-MS (Method A): Rt=1.23 min

MS (ESIpos): m/z=457 (M+H)⁺

¹H-NMR (400 MHz, DMSO-d₆) δ [ppm]: 2.07 (s, 3H), 3.82 (s, 2H), 5.10-5.13 (m, 1H), 5.22-5.24 (m, 1H), 6.61 (d, 1H), 6.95 (dd, 1H), 7.21-7.47 (m, 9H), 7.66 (dd, 1H), 8.16 (d, 1H), 10.42 (s, 1H).

### Example 179

### N-[4-(3-Chlorophenoxy)-3-sulfamoylphenyl]-2-(4-methylphenyl)acetamide

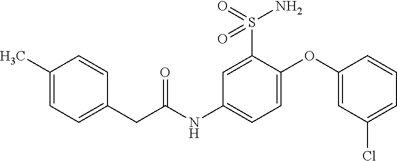 [SMILES: Cc1ccc(CC(=O)Nc2ccc(Oc3cccc(Cl)c3)c(S(N)(=O)=O)c2)cc1]

According to GP3.4 and GP4 5-amino-2-(3-chlorophenoxy)-N-(2,4-dimethoxybenzyl)benzenesulfonamide (150 mg, 0.267 mmol) and (4-methylphenyl)acetyl chloride (50 mg, 0.294 mmol) were reacted to N-[4-(3-chlorophenoxy)-3-sulfamoylphenyl]-2-(4-methylphenyl)acetamide. The pure compound was obtained after purification by preparative HPLC (Waters XBrigde C18 5μ 100×30 mm, acetonitrile/water+0.2% aqueous ammonia (32%)) (23 mg, 0.053 mmol, 21% yield over 2 steps, 95% purity).

LC-MS (Method E): Rt=1.18 min

MS (ESIpos): m/z=431 (M+H)⁺

¹H-NMR (DMSO-d₆) δ [ppm]: 2.27 (s, 3H), 3.59 (s, 2H), 6.94-7.00 (m, 1H), 7.05-7.09 (m, 2H), 7.11-7.15 (m, 2H), 7.17-7.24 (m, 3H), 7.32-7.43 (m, 3H), 7.75-7.85 (m, 1H), 8.15-8.23 (m, 1H), 10.39 (s, 1H).

### Example 180

### N-[4-(3-Chlorophenoxy)-3-sulfamoylphenyl]-2-(4-chlorophenyl)acetamide

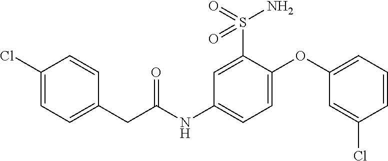 [SMILES: NS(=O)(=O)c1cc(NC(=O)Cc2ccc(Cl)cc2)ccc1Oc1cccc(Cl)c1]

According to GP3.4 and GP4 5-amino-2-(3-chlorophenoxy)-N-(2,4-dimethoxybenzyl)benzenesulfonamide (150 mg, 0.267 mmol) and (4-chlorophenyl)acetyl chloride (56 mg, 0.294 mmol) were reacted to N-[4-(3-chlorophenoxy)-3-sulfamoylphenyl]-2-(4-chlorophenyl)acetamide. The pure compound was obtained after preparative HPLC (Waters XBrigde C18 5μ 100×30 mm, acetonitrile/water+0.2% aqueous ammonia (32%)) (24 mg, 0.054 mmol, 22% yield over 2 steps, 95% purity).

LC-MS (Method E): Rt=1.20 min

MS (ESIpos): m/z=451 (M+H)⁺

¹H-NMR (DMSO-d₆) δ [ppm]: 3.66 (s, 2H), 6.90-7.02 (m, 1H), 7.05-7.13 (m, 2H), 7.14-7.23 (m, 1H), 7.35-7.42 (m, 7H), 7.77-7.84 (m, 1H), 8.14-8.23 (m, 1H), 10.48 (s, 1H).

### Example 181

### N-[4-(3-Chlorophenoxy)-3-sulfamoylphenyl]-2-(pyridin-3-yl)acetamide

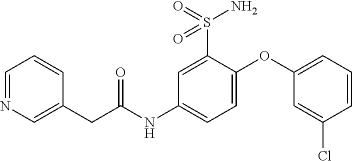 [SMILES: NS(=O)(=O)c1cc(NC(=O)Cc2cccnc2)ccc1Oc1cccc(Cl)c1]

According to GP4 N-4-(3-chlorophenoxy)-3-[(2,4-dimethoxybenzyl)sulfamoyl]phenyl-2-(pyridin-3-yl)acetamide (150 mg, 0.264 mmol) was converted to N-[4-(3-chlorophenoxy)-3-sulfamoylphenyl]-2-(pyridin-3-yl)acetamide. The pure compound was obtained after preparative HPLC (Waters XBrigde C18 5μ 100×30 mm, acetonitrile/water+0.2% aqueous ammonia (32%)) (10 mg, 0.025 mmol, 9% yield, 95% purity).

LC-MS (Method E): Rt=0.89 min

MS (ESIpos): m/z=418 (M+H)⁺

¹H-NMR (DMSO-d₆) δ [ppm]: 3.71 (s, 2H), 6.95-7.00 (m, 1H), 7.06-7.11 (m, 2H), 7.17-7.22 (m, 1H), 7.33-7.42 (m, 4H), 7.72-7.83 (m, 2H), 8.19 (d, 1H), 8.44-8.49 (m, 1H), 8.51-8.55 (m, 1H), 10.53 (s, 1H).

### Example 182

### N-[4-(3-Chlorophenoxy)-3-sulfamoylphenyl]-2-(2-methylphenyl)acetamide

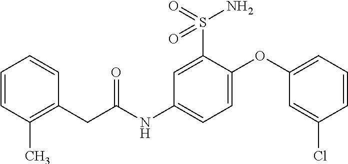 [SMILES: Cc1ccccc1CC(=O)Nc1ccc(Oc2cccc(Cl)c2)c(S(N)(=O)=O)c1]

According to GP3.4 and GP4 5-amino-2-(3-chlorophenoxy)-N-(2,4-dimethoxybenzyl)benzenesulfonamide (150 mg, 0.267 mmol) was reacted with (2-methylphenyl)acetyl chloride (50 mg, 0.294 mmol) to N-[4-(3-chlorophenoxy)-3-sulfamoylphenyl]-2-(2-methylphenyl)acetamide. The pure compound was obtained after preparative HPLC (Waters XBrigde C18 5μ 100×30 mm, acetonitrile/water+0.2% aqueous ammonia (32%)) (15 mg, 0.034 mmol, 13% yield over 2 steps, 95% purity).

LC-MS (Method E): Rt=1.17 min

MS (ESIneg): m/z=429 (M−H)+

¹H-NMR (DMSO-d₆) δ [ppm]: 2.30 (s, 3H), 3.68 (s, 2H), 6.93-7.03 (m, 1H), 7.05-7.28 (m, 7H), 7.34-7.45 (m, 3H), 7.76-7.86 (m, 1H), 8.16-8.25 (m, 1H), 10.44 (s, 1H).

### Example 183

### N-[4-(3-Chlorophenoxy)-3-sulfamoylphenyl]-2-(3-methylphenyl)acetamide

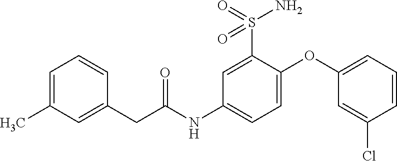 [SMILES: Cc1cccc(CC(=O)Nc2ccc(Oc3cccc(Cl)c3)c(S(N)(=O)=O)c2)c1]

According to GP3.4 and GP4 5-amino-2-(3-chlorophenoxy)-N-(2,4-dimethoxybenzyl)benzenesulfonamide (150 mg, 0.267 mmol) was reacted with (3-methylphenyl)acetyl chloride (50 mg, 0.294 mmol) to N-[4-(3-chlorophenoxy)-3-sulfamoylphenyl]-2-(3-methylphenyl)acetamide. The pure compound was obtained after preparative HPLC (Waters XBrigde C18 5μ 100×30 mm, acetonitrile/water+0.2% aqueous ammonia (32%)) (22 mg, 0.052 mmol, 20% yield over 2 steps, 95% purity).

LC-MS (Method E): Rt=1.18 min

MS (ESIneg): m/z=429 (M−H)⁺

¹H-NMR (DMSO-d₆) δ [ppm]: 2.28 (s, 3H), 3.59 (s, 2H), 6.92-7.01 (m, 1H), 7.03-7.26 (m, 7H), 7.33-7.44 (m, 3H), 7.77-7.87 (m, 1H), 8.15-8.24 (m, 1H), 10.44 (s, 1H).

### Example 184

### N-[4-(3-Chlorophenoxy)-3-sulfamoylphenyl]-2-phenylpropanamide

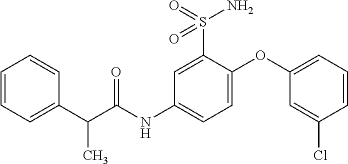 [SMILES: CC(C(=O)Nc1ccc(Oc2cccc(Cl)c2)c(S(N)(=O)=O)c1)c1ccccc1]

According to GP3.4 and GP4 5-amino-2-(3-chlorophenoxy)-N-(2,4-dimethoxybenzyl)benzenesulfonamide (170 mg, 0.284 mmol) and 2-phenylpropanoyl chloride (57 mg, 0.340 mmol) were reacted to N-[4-(3-chlorophenoxy)-3-sulfamoylphenyl]-2-phenylpropanamide. The pure compound was obtained after preparative HPLC (Waters XBrigde C18 5μ 100×30 mm, acetonitrile/water+0.2% aqueous ammonia (32%)) (17 mg, 0.038 mmol, 15% yield over 2 steps, 95% yield).

LC-MS (Method E): Rt=1.17 min

MS (ESIneg): m/z=429 (M−H)⁺

¹H-NMR (DMSO-d₆) δ [ppm]: 1.42 (d, 3H), 3.82 (q, 1H), 6.96 (dd, 1H), 7.03-7.10 (m, 2H), 7.16-7.20 (m, 1H), 7.21-7.27 (m, 1H), 7.31-7.43 (m, 7H), 7.81 (dd, 1H), 8.20 (d, 1H), 10.35 (s, 1H).

### Example 185

### N-[4-(3-Chlorophenoxy)-3-sulfamoylphenyl]-2-(pyridin-2-yl)acetamide

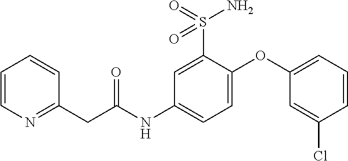 [SMILES: NS(=O)(=O)c1cc(NC(=O)Cc2ccccn2)ccc1Oc1cccc(Cl)c1]

According to GP4 N-4-(3-chlorophenoxy)-3-[(2,4-dimethoxybenzyl)sulfamoyl]phenyl-2-(pyridin-2-yl)acetamide (260 mg, 0.457 mmol) was converted to N-[4-(3-chlorophenoxy)-3-sulfamoylphenyl]-2-(pyridin-2-yl)acetamide. The pure compound was obtained by preparative HPLC (Waters XBrigde C18 5μ 100×30 mm, acetonitrile/water+0.2% aqueous ammonia (32%)) (80 mg, 0.230 mmol, 51% yield, 95% purity).

LC-MS (Method E): Rt=0.92 min

MS (ESIneg): m/z=416 (M−H)⁺

¹H-NMR (DMSO-d₆) δ [ppm]: 3.85 (s, 2H), 6.90-7.05 (m, 1H), 7.06-7.15 (m, 2H), 7.15-7.25 (m, 1H), 7.25-7.34 (m, 1H), 7.35-7.48 (m, 4H), 7.67-7.89 (m, 2H), 8.23 (d, 1H), 8.50 (dd, 1H), 10.56 (s, 1H).

### Example 186

### N-[4-(3-Chlorophenoxy)-3-sulfamoylphenyl]-2-(3-chlorophenyl)acetamide

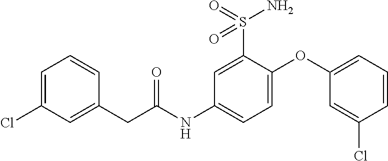 [SMILES: NS(=O)(=O)c1cc(NC(=O)Cc2cccc(Cl)c2)ccc1Oc1cccc(Cl)c1]

According to GP4 N-4-(3-chlorophenoxy)-3-[(2,4-dimethoxybenzyl)sulfamoyl]phenyl-2-(4-chlorophenyl)acetamide (520 mg, 0.086 mmol) was converted to N-[4-(3-chlorophenoxy)-3-sulfamoylphenyl]-2-(3-chlorophenyl)acetamide. The pure compound was obtained after preparative HPLC (Waters XBrigde C18 5μ 100×30 mm, acetonitrile/water+0.2% aqueous ammonia (32%)) (12 mg, 0.026 mmol, 30% yield, 95% purity).

LC-MS (Method E): Rt=1.19 min

MS (ESIneg): m/z=449 (M−H)⁺

¹H-NMR (DMSO-d₆) δ [ppm]: 3.68 (s, 2H), 6.95-6.99 (m, 1H), 7.06-7.10 (m, 2H), 7.17-7.22 (m, 1H), 7.28-7.43 (m, 7H), 7.80 (dd, 1H), 8.19 (d, 1H), 10.49 (s, 1H).

### Example 187

### N-[4-(3-Chlorophenoxy)-3-sulfamoylphenyl]-2-(2-chlorophenyl)acetamide

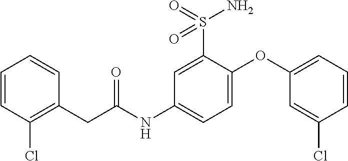 [SMILES: NS(=O)(=O)c1cc(NC(=O)Cc2ccccc2Cl)ccc1Oc1cccc(Cl)c1]

According to GP3.4 and GP4 5-amino-2-(3-chlorophenoxy)-N-(2,4-dimethoxybenzyl)benzenesulfonamide (175 mg, 0.292 mmol) and (2-chlorophenyl)acetyl chloride (66 mg, 0.350 mmol) were reacted to N-[4-(3-chlorophenoxy)-3-sulfamoylphenyl]-2-(2-chlorophenyl)acetamide. The pure compound was obtained after preparative HPLC (Waters XBrigde C18 5μ 100×30 mm, acetonitrile/water+0.2% aqueous ammonia (32%)) (34 mg, 0.075 mmol, 26% yield, 95% purity).

LC-MS (Method E): Rt=1.16 min

MS (ESIneg): m/z=449 (M−H)⁺

¹H-NMR (DMSO-d₆) δ [ppm]: 3.86 (s, 2H), 6.92-7.14 (m, 3H), 7.17-7.23 (m, 1H), 7.27-7.52 (m, 7H), 7.73-7.86 (m, 1H), 8.17-8.28 (m, 1H), 10.53 (s, 1H).

### Example 188

### N-[4-(3-Chlorophenoxy)-3-sulfamoylphenyl]-2-(pyridin-4-yl)acetamide

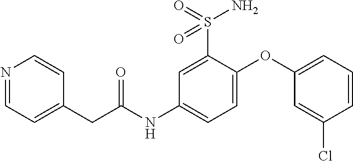 [SMILES: NS(=O)(=O)c1cc(NC(=O)Cc2ccncc2)ccc1Oc1cccc(Cl)c1]

According to GP4 N-4-(3-chlorophenoxy)-3-[(2,4-dimethoxybenzyl)sulfamoyl]phenyl-2-(pyridin-4-yl)acetamide (70 mg, 0.113 mmol) was converted to N-[4-(3-chlorophenoxy)-3-sulfamoylphenyl]-2-(pyridin-4-yl)acetamide and purified by preparative HPLC (Waters XBrigde C18 5μ 100×30 mm, acetonitrile/water+0.2% aqueous ammonia (32%)) (7.3 mg, 0.017 mmol, 15% yield, 95% purity).

LC-MS (Method E): Rt=0.86 min

MS (ESIneg): m/z=416 (M−H)⁺

¹H-NMR (DMSO-d₆) δ [ppm]: 3.72 (s, 2H), 6.94-7.00 (m, 1H), 7.05-7.11 (m, 2H), 7.17-7.22 (m, 1H), 7.31-7.43 (m, 5H), 7.80 (dd, 1H), 8.19 (d, 1H), 8.48-8.54 (m, 2H), 10.55 (s, 1H).

### Example 189

### N-[4-(3-Chlorophenoxy)-3-sulfamoylphenyl]-2-(6-methylpyridin-2-yl)acetamide

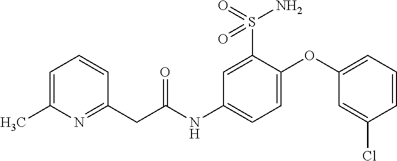 [SMILES: Cc1cccc(CC(=O)Nc2ccc(Oc3cccc(Cl)c3)c(S(N)(=O)=O)c2)n1]

According to GP4 N-4-(3-chlorophenoxy)-3-[(2,4-dimethoxybenzyl)sulfamoyl]phenyl-2-(2-chlorophenyl)acetamide (70 mg, 0.111 mmol) was converted to N-[4-(3-chlorophenoxy)-3-sulfamoylphenyl]-2-(6-methylpyridin-2-yl)acetamide. The pure compound was obtained after preparative HPLC (Waters XBrigde C18 5μ 100×30 mm, acetonitrile/water+0.2% aqueous ammonia (32%)) (24 mg, 0.056 mmol, 51% yield, 95% purity).

LC-MS (Method E): Rt=1.00 min

MS (ESIneg): m/z=430 (M−H)⁺

¹H-NMR (DMSO-d₆) δ [ppm]: 2.44 (s, 3H), 3.80 (s, 2H), 6.92-7.01 (m, 1H), 7.05-7.22 (m, 5H), 7.34-7.47 (m, 3H), 7.61-7.70 (m, 1H), 7.80 (dd, 1H), 8.23 (d, 1H), 10.55 (s, 1H).

### Example 190

### N-[4-(3-Chlorophenoxy)-3-sulfamoylphenyl]-2-(4-methoxyphenyl)acetamide

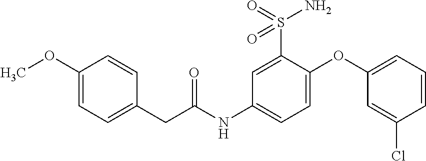 [SMILES: COc1ccc(CC(=O)Nc2ccc(Oc3cccc(Cl)c3)c(S(N)(=O)=O)c2)cc1]

According to GP4 N-4-(3-chlorophenoxy)-3-[(2,4-dimethoxybenzyl)sulfamoyl]phenyl-2-(4-methoxyphenyl)acetamide (70 mg, 0.108 mmol) was converted to N-[4-(3-chlorophenoxy)-3-sulfamoylphenyl]-2-(4-methoxyphenyl)acetamide. The pure compound was obtained after preparative HPLC (Waters XBrigde C18 5μ 100×30 mm, acetonitrile/water+0.2% aqueous ammonia (32%)) (31 mg, 0.069 mmol, 64% yield, 95% purity).

LC-MS (Method E): Rt=1.09 min

MS (ESIneg): m/z=445 (M−H)⁺

¹H-NMR (DMSO-d₆) δ [ppm]: 3.56 (s, 2H), 3.73 (s, 3H), 6.84-6.93 (m, 2H), 6.94-7.00 (m, 1H), 7.04-7.11 (m, 2H), 7.15-7.22 (m, 1H), 7.22-7.29 (m, 2H), 7.34-7.42 (m, 3H), 7.80 (dd, 1H), 8.18 (d, 1H), 10.41 (s, 1H).

### Example 191

### N-[4-(3-Chlorophenoxy)-3-sulfamoylphenyl]-2-(3-methoxyphenyl)acetamide

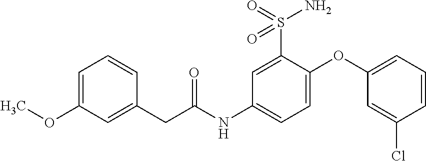 [SMILES: COc1cccc(CC(=O)Nc2ccc(Oc3cccc(Cl)c3)c(S(N)(=O)=O)c2)c1]

According to GP4 N-4-(3-chlorophenoxy)-3-[(2,4-dimethoxybenzyl)sulfamoyl]phenyl-2-(3-methoxyphenyl)acetamide (70 mg, 0.108 mmol) was converted to N-[4-(3-chlorophenoxy)-3-sulfamoylphenyl]-2-(3-methoxyphenyl)acetamide. The pure compound was obtained after preparative HPLC (Waters XBrigde C18 5μ 100×30 mm, acetonitrile/water+0.2% aqueous ammonia (32%)) (17 mg, 0.037 mmol, 48% yield, 95% purity).

LC-MS (Method E): Rt=1.09 min

MS (ESIneg): m/z=445 (M−H)⁺

¹H-NMR (DMSO-d₆) δ [ppm]: 3.61 (s, 2H), 3.73 (s, 3H), 6.78-6.87 (m, 1H), 6.87-6.94 (m, 2H), 6.94-7.01 (m, 1H), 7.03-7.15 (m, 2H), 7.15-7.29 (m, 2H), 7.33-7.45 (m, 3H), 7.81 (dd, 1H), 8.19 (d, 1H), 10.44 (s, 1H).

### Example 192

### N-[4-(3-Chlorophenoxy)-3-sulfamoylphenyl]-2-(2-methoxyphenyl)acetamide

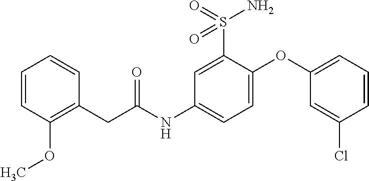 [SMILES: COc1ccccc1CC(=O)Nc1ccc(Oc2cccc(Cl)c2)c(S(N)(=O)=O)c1]

According to GP4 N-4-(3-chlorophenoxy)-3-[(2,4-dimethoxybenzyl)sulfamoyl]phenyl-2-(2-methoxyphenyl)acetamide (70 mg, 0.108 mmol) were converted to N-[4-(3-chlorophenoxy)-3-sulfamoylphenyl]-2-(2-methoxyphenyl)acetamide. The pure compound was obtained after preparative HPLC (Waters XBrigde C18 5μ 100×30 mm, acetonitrile/water+0.2% aqueous ammonia (32%)) (40 mg, 0.090 mmol, 84%, 95% purity).

LC-MS (Method E): Rt=1.11 min

MS (ESIneg): m/z=445 (M−H)⁺

¹H-NMR (DMSO-d₆) δ [ppm]: 3.63 (s, 2H), 3.77 (s, 3H), 6.86-6.95 (m, 1H), 6.95-7.03 (m, 2H), 7.06-7.13 (m, 2H), 7.14-7.31 (m, 3H), 7.34-7.44 (m, 3H), 7.80 (dd, 1H), 8.22 (d, 1H), 10.36 (s, 1H).

### Example 193

### N-[4-(3-Chlorophenoxy)-3-sulfamoylphenyl]-2-(5-methylpyridin-2-yl)acetamide

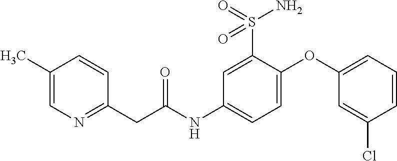 [SMILES: Cc1ccc(CC(=O)Nc2ccc(Oc3cccc(Cl)c3)c(S(N)(=O)=O)c2)nc1]

According to GP4 N-{4-(3-chlorophenoxy)-3-[(2,4-dimethoxybenzyl)sulfamoyl]phenyl}-2-(5-methylpyridin-2-yl)acetamide (200 mg, 0.344 mmol) was reacted to N-[4-(3-chlorophenoxy)-3-sulfamoylphenyl]-2-(5-methylpyridin-2-yl)acetamide. The pure compound was obtained after preparative HPLC (Waters XBrigde C18 5μ 100×30 mm, acetonitrile/water+0.2% aqueous ammonia (32%)) (16 mg, 0.037 mmol, 11% yield, 95% purity).

LC-MS (Method E): Rt=0.87 min

MS (ESIneg): m/z=430 (M−H)⁺

¹H-NMR (DMSO-d₆) δ [ppm]: 2.27 (s, 3H), 3.80 (s, 2H), 6.93-7.03 (m, 1H), 7.05-7.13 (m, 2H), 7.15-7.22 (m, 1H), 7.24-7.33 (m, 1H), 7.36-7.45 (m, 3H), 7.52-7.64 (m, 1H), 7.74-7.87 (m, 1H), 8.18-8.26 (m, 1H), 8.30-8.38 (m, 1H), 10.51 (s, 1H).

### Example 194

### (2S)—N-[4-(3-chlorophenoxy)-3-sulfamoylphenyl]-2-phenylpropanamide

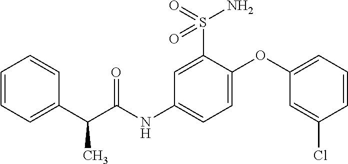 [SMILES: C[C@H](C(=O)Nc1ccc(Oc2cccc(Cl)c2)c(S(N)(=O)=O)c1)c1ccccc1]

5-Amino-2-(3-chlorophenoxy)-N-(2,4-dimethoxybenzyl)benzenesulfonamide (250 mg, 0.557 mmol) and (2S)-2-phenylpropanoic acid (100 mg, 0.668 mmol) were reacted according to GP3.2 and GP4 to (2S)—N-[4-(3-chlorophenoxy)-3-sulfamoylphenyl]-2-phenylpropanamide. The pure compound was obtained after preparative HPLC (Waters XBrigde C18 5μ 100×30 mm, acetonitrile/water+0.2% aqueous ammonia (32%)) (114 mg, 0.264 mmol, 47% yield over 2 steps, 95% purity).

LC-MS (Method E): Rt=1.17 min

MS (ESIneg): m/z=429 (M−H)

[α]_{D}²⁰ 49.6+/−0.28 (c=5.1 mg/mL, DMSO)

¹H-NMR (DMSO-d₆) δ [ppm]: 1.42 (d, 3H), 3.77-3.86 (m, 1H), 6.89-6.99 (m, 1H), 7.03-7.09 (m, 2H), 7.14-7.21 (m, 1H), 7.21-7.28 (m, 1H), 7.29-7.43 (m, 7H), 7.76-7.84 (m, 1H), 8.20 (d, 1H), 10.34 (s, 1H).

### Example 195

### (2R)—N-[4-(3-Chlorophenoxy)-3-sulfamoylphenyl]-2-phenylpropanamide

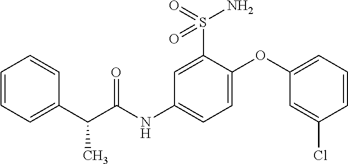 [SMILES: C[C@@H](C(=O)Nc1ccc(Oc2cccc(Cl)c2)c(S(N)(=O)=O)c1)c1ccccc1]

According to GP3.2 and GP4 5-amino-2-(3-chlorophenoxy)-N-(2,4-dimethoxybenzyl)benzenesulfonamide (250 mg, 0.557 mmol) and (2R)-2-phenylpropanoic acid (100 mg, 0.668 mmol) were converted to (2R)—N-[4-(3-chlorophenoxy)-3-sulfamoylphenyl]-2-phenylpropanamide. The pure compound was obtained after preparative HPLC (Waters XBrigde C18 5μ 100×30 mm, acetonitrile/water+0.2% aqueous ammonia (32%)) (114 mg, 0.266 mmol, 48% yield, 95% purity).

LC-MS (Method E): Rt=1.18 min

MS (ESIneg): m/z=429 (M−H)⁺

[α]_{D}²⁰ −41.7°+/−0.88° (c=5.0 mg/mL, DMSO)

¹H-NMR (DMSO-d₆) δ [ppm]: 1.42 (d, 3H), 3.82 (q, 1H), 6.92-7.00 (m, 1H), 7.04-7.11 (m, 2H), 7.15-7.21 (m, 1H), 7.21-7.29 (m, 1H), 7.30-7.44 (m, 7H), 7.81 (dd, 1H), 8.20 (d, 1H), 10.35 (s, 1H).

### Example 196

### N-[4-(3-Chlorophenoxy)-3-sulfamoylphenyl]-2-(2-chlorophenyl)propanamide

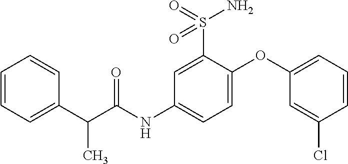 [SMILES: CC(C(=O)Nc1ccc(Oc2cccc(Cl)c2)c(S(N)(=O)=O)c1)c1ccccc1]

According to GP3.2 and GP4 5-amino-2-(3-chlorophenoxy)-N-(2,4-dimethoxybenzyl)benzenesulfonamide (170 mg, 0.379 mmol) was reacted with (2-(2-chlorophenyl)propanoic acid (84 mg, 0.454 mmol) to N-[4-(3-chlorophenoxy)-3-sulfamoylphenyl]-2-(2-chlorophenyl)propanamide and was purified by preparative HPLC (Waters XBrigde C18 5μ 100×30 mm, acetonitrile/water+0.2% aqueous ammonia (32%)) (24 mg, 0.048 mmol, 13% yield, 95% purity)

LC-MS (Method E): Rt=1.26 min

MS (ESIneg): m/z=463 (M−H)⁺

¹H-NMR (DMSO-d₆) δ [ppm]: 1.45 (d, 3H), 4.20 (q, 1H), 6.94-7.00 (m, 1H), 7.05-7.11 (m, 2H), 7.19 (ddd, 1H), 7.27-7.42 (m, 5H), 7.46 (dd, 2H), 7.83 (dd, 1H), 8.22 (d, 1H), 10.43 (s, 1H).

### Example 197

### 2-(2-{[4-(3-Chlorophenoxy)-3-sulfamoylphenyl]amino}-2-oxoethyl)-N-(2-methoxyethyl)-N-methylbenzamide

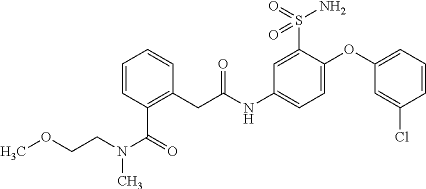 [SMILES: COCCN(C)C(=O)c1ccccc1CC(=O)Nc1ccc(Oc2cccc(Cl)c2)c(S(N)(=O)=O)c1]

According to GP3.2 and GP4 5-amino-2-(3-chlorophenoxy)-N-(2,4-dimethoxybenzyl)benzenesulfonamide (67 mg, 0.149 mmol) was reacted with {2-[(2-methoxyethyl)(methyl)carbamoyl]phenyl}acetic acid (45 mg, 0.180 mmol) to 2-(2-{[4-(3-chlorophenoxy)-3-sulfamoylphenyl]amino}-2-oxoethyl)-N-(2-methoxyethyl)-N-methylbenzamide and was purified by preparative HPLC (Waters XBrigde C18 5μ 100×30 mm, acetonitrile/water+0.2% aqueous ammonia (32%)) (17 mg, 0.033 mmol, 20% yield over 2 steps, 95% purity).

LC-MS (Method E): Rt=1.10 min

MS (ESIneg): m/z=530 (M−H)⁺

### Example 198

### 2-(2-{[4-(3-Chlorophenoxy)-3-sulfamoylphenyl]amino}-2-oxoethyl)-N,N-dimethylbenzamide

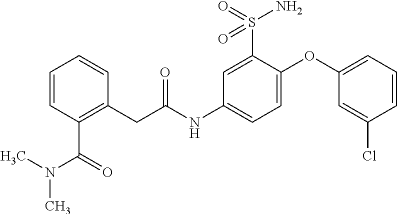 [SMILES: CN(C)C(=O)c1ccccc1CC(=O)Nc1ccc(Oc2cccc(Cl)c2)c(S(N)(=O)=O)c1]

According to GP3.2 and GP4 (5-amino-2-(3-chlorophenoxy)-N-(2,4-dimethoxybenzyl)benzenesulfonamide (100 mg, 0.223 mmol) and [2-(dimethylcarbamoyl)phenyl]acetic acid (55 mg, 0.267 mmol) were reacted to 2-(2-{[4-(3-chlorophenoxy)-3-sulfamoylphenyl]amino}-2-oxoethyl)-N,N-dimethylbenzamide. The pure compound was obtained after preparative HPLC (Waters XBrigde C18 5μ 100×30 mm, acetonitrile/water+0.2% aqueous ammonia (32%)) (58 mg, 0.118 mmol, 50% yield over 2 steps, 95% purity).

LC-MS (Method E): Rt=1.06 min

MS (ESIneg): m/z=486 (M−H)⁺

¹H-NMR (DMSO-d₆) δ [ppm]: 2.77 (s, 3H), 2.97 (s, 3H), 3.68 (s, 2H), 6.91-7.04 (m, 1H), 7.04-7.15 (m, 2H), 7.16-7.27 (m, 2H), 7.28-7.46 (m, 6H), 7.70-7.83 (m, 1H), 8.12-8.23 (m, 1H), 10.46 (s, 1H).

### Example 199

### N-[4-(Cyclohexyloxy)-3-sulfamoylphenyl]-2-phenylacetamide

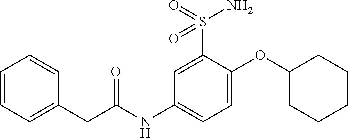 [SMILES: NS(=O)(=O)c1cc(NC(=O)Cc2ccccc2)ccc1OC1CCCCC1]

According to GP3.2 5-amino-2-(cyclohexyloxy)benzenesulfonamide (180 mg, 0.7 mmol) and phenylacetic acid (109 mg, 0.8 mmol) were converted. Pure N-[4-(cyclohexyloxy)-3-sulfamoylphenyl]-2-phenylacetamide was obtained after preparative HPLC (Waters XBrigde C18 5μ 100×30 mm, acetonitrile/water+0.1% formic acid) (107 mg, 0.27 mmol, 40% yield, 99% purity).

LC-MS (Method D): Rt=1.14 min

MS (ESIneg): m/z=387 (M−H)⁺

¹H-NMR (DMSO-d₆) δ [ppm]: 1.21-1.44 (m, 3H), 1.44-1.54 (m, 1H), 1.54-1.66 (m, 2H), 1.66-1.79 (m, 2H), 1.79-1.96 (m, 2H), 3.60 (s, 2H), 4.37-4.56 (m, 1H), 6.75 (s, 2H), 7.16-7.27 (m, 2H), 7.32 (d, 4H), 7.74 (dd, 1H), 8.01 (d, 1H), 10.24 (s, 1H).

### Example 200

### 2-(2-Chlorophenyl)-N-[4-(cyclohexyloxy)-3-sulfamoylphenyl]acetamide

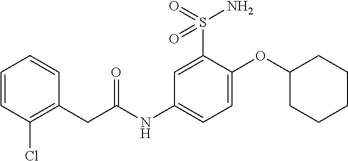 [SMILES: NS(=O)(=O)c1cc(NC(=O)Cc2ccccc2Cl)ccc1OC1CCCCC1]

According to GP3.2 5-amino-2-(cyclohexyloxy)benzenesulfonamide (180 mg, 0.7 mmol) and (2-chlorophenyl)acetic acid (136 mg, 0.8 mmol) were converted. Pure 2-(2-chlorophenyl)-N-[4-(cyclohexyloxy)-3-sulfamoylphenyl]acetamide was obtained after preparative HPLC (Waters XBrigde C18 5μ 100×30 mm, acetonitrile/water+0.1% formic acid) (155 mg, 0.37 mmol, 55% yield, 99% yield)

LC-MS (Method A): Rt=1.16 min

MS (ESIpos): m/z=422 (M+H)⁺

¹H-NMR (DMSO-d₆) δ [ppm]: 1.16-1.42 (m, 3H), 1.44-1.56 (m, 1H), 1.57-1.69 (m, 2H), 1.69-1.82 (m, 2H), 1.83-1.96 (m, 2H), 3.81 (s, 2H), 4.41-4.57 (m, 1H), 6.76 (s, 2H), 7.20 (d, 1H), 7.26-7.36 (m, 2H), 7.38-7.51 (m, 2H), 7.74 (dd, 1H), 8.03 (d, 1H), 10.29 (s, 1H).

### Example 201

### 3-(2-{[4-(3-Chlorophenoxy)-3-sulfamoylphenyl]amino}-2-oxoethyl)-N-(2-methoxyethyl)benzamide

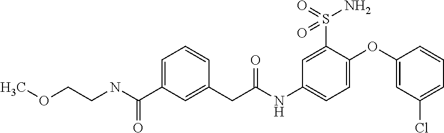 [SMILES: COCCNC(=O)c1cccc(CC(=O)Nc2ccc(Oc3cccc(Cl)c3)c(S(N)(=O)=O)c2)c1]

According to GP3.2 and GP4 (5-amino-2-(3-chlorophenoxy)-N-(2,4-dimethoxybenzyl)benzenesulfonamide (102 mg, 0.228 mmol) and {3-[(2-methoxyethyl)carbamoyl]phenyl}acetic acid (65 mg, 0.273 mmol) were reacted to 3-(2-{[4-(3-chlorophenoxy)-3-sulfamoylphenyl]amino}-2-oxoethyl)-N-(2-methoxyethyl)benzamide. The pure compound was obtained after preparative HPLC (Waters XBrigde C18 5μ 100×30 mm, acetonitrile/water+0.2% aqueous ammonia (32%)) (46 mg, 0.089 mmol, 37% yield over 2 steps, 95% purity).

LC-MS (Method E): Rt=0.95 min

MS (ESIneg): m/z=516 (M−H)⁺

¹H-NMR (DMSO-d₆) δ [ppm]: 3.25 (s, 3H), 3.38-3.51 (m, 4H), 3.71 (s, 2H), 6.90-7.03 (m, 1H), 7.03-7.10 (m, 2H), 7.15-7.23 (m, 1H), 7.29-7.45 (m, 4H), 7.44-7.52 (m, 1H), 7.68-7.78 (m, 1H), 7.79-7.87 (m, 2H), 8.14-8.25 (m, 1H), 8.45-8.54 (m, 1H), 10.49 (s, 1H).

### Example 202

### 3-(2-{[4-(3-Chlorophenoxy)-3-sulfamoylphenyl]amino}-2-oxoethyl)-N,N-dimethylbenzamide

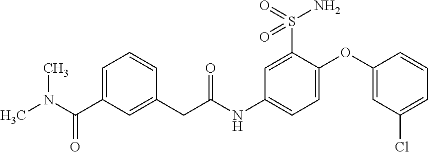 [SMILES: CN(C)C(=O)c1cccc(CC(=O)Nc2ccc(Oc3cccc(Cl)c3)c(S(N)(=O)=O)c2)c1]

According to GP3.2 and GP4 5-amino-2-(3-chlorophenoxy)-N-(2,4-dimethoxybenzyl)benzenesulfonamide (67 mg, 0.149 mmol) and [3-(dimethylcarbamoyl)phenyl]acetic acid (37 mg, 0.179 mmol) were reacted to 3-(2-{[4-(3-chlorophenoxy)-3-sulfamoylphenyl]amino}-2-oxoethyl)-N,N-dimethylbenzamide. The pure compound was obtained after preparative HPLC (Waters XBrigde C18 5μ 100×30 mm, acetonitrile/water+0.2% aqueous ammonia (32%)) (11 mg, 0.022 mmol, 9% yield over 2 steps, 95% purity).

LC-MS (Method E): Rt=0.96 min

MS (ESIneg): m/z=486 (M−H)⁺

¹H-NMR (DMSO-d₆) δ [ppm]: 2.86-3.04 (m, 6H), 3.70 (s, 2H), 6.92-7.03 (m, 1H), 7.03-7.13 (m, 2H), 7.15-7.25 (m, 1H), 7.25-7.34 (m, 1H), 7.34-7.48 (m, 6H), 7.73-7.88 (m, 1H), 8.20 (d, 1H), 10.49 (s, 1H).

### Example 203

### 3-(2-{[4-(3-Chlorophenoxy)-3-sulfamoylphenyl]amino}-2-oxoethyl)-N-methylbenzamide

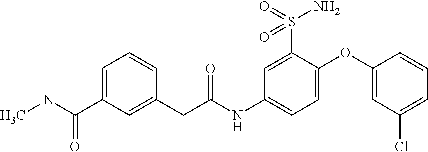 [SMILES: CNC(=O)c1cccc(CC(=O)Nc2ccc(Oc3cccc(Cl)c3)c(S(N)(=O)=O)c2)c1]

According to GP3.2 and GP4 5-amino-2-(3-chlorophenoxy)-N-(2,4-dimethoxybenzyl)benzenesulfonamide (100 mg, 0.223 mmol) and [3-(methylcarbamoyl)phenyl]acetic acid (52 mg, 0.267 mmol) were reacted to 3-(2-{[4-(3-chlorophenoxy)-3-sulfamoylphenyl]amino}-2-oxoethyl)-N-methylbenzamide. The pure compound was obtained after preparative HPLC (Waters XBrigde C18 5μ 100×30 mm, acetonitrile/water+0.2% aqueous ammonia (32%)) (34 mg, 0.072 mmol, 28% yield over 2 steps, 95% purity).

LC-MS (Method E): Rt=0.90 min

MS (ESIneg): m/z=472 (M−H)⁺

¹H-NMR (DMSO-d₆) δ [ppm]: 2.78 (d, 3H), 3.71 (s, 2H), 6.92-7.00 (m, 1H), 7.04-7.12 (m, 2H), 7.16-7.23 (m, 1H), 7.34-7.54 (m, 5H), 7.65-7.75 (m, 1H), 7.78-7.88 (m, 2H), 8.20 (d, 1H), 8.36-8.48 (m, 1H), 10.51 (s, 1H).

### Example 204

### N-[4-(Cyclobutyloxy)-3-sulfamoylphenyl]-2-phenylacetamide

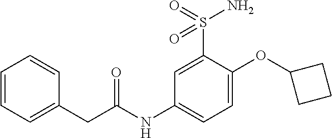 [SMILES: NS(=O)(=O)c1cc(NC(=O)Cc2ccccc2)ccc1OC1CCC1]

According to GP3.2 5-amino-2-(cyclobutyloxy)benzenesulfonamide (156 mg, 0.6 mmol) was reacted with phenylacetic acid (105 mg, 0.8 mmol). Pure N-[4-(cyclobutyloxy)-3-sulfamoylphenyl]-2-phenylacetamide was obtained after preparative HPLC (Waters XBrigde C18 5μ 100×30 mm, methanol/water+0.1% formic acid) (140 mg, 0.39 mmol, 60% yield, 99% purity).

LC-MS (Method H): Rt=1.16 min

MS (ESIpos): m/z=360 (M+H)⁺

¹H-NMR (DMSO-d₆) δ [ppm]: 1.49-1.70 (m, 1H), 1.70-1.86 (m, 1H), 2.09-2.29 (m, 2H), 2.29-2.45 (m, 2H), 3.60 (s, 2H), 4.79 (t, 1H), 6.85-7.05 (m, 3H), 7.17-7.41 (m, 5H), 7.74 (dd, 1H), 8.01 (d, 1H), 10.25 (s, 1H).

### Example 205

### 2-(2-Chlorophenyl)-N-[4-(cyclobutyloxy)-3-sulfamoylphenyl]acetamide

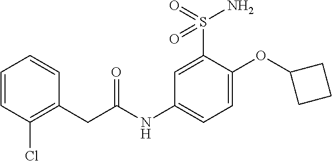 [SMILES: NS(=O)(=O)c1cc(NC(=O)Cc2ccccc2Cl)ccc1OC1CCC1]

According to GP3.2 5-amino-2-(cyclobutyloxy)benzenesulfonamide (156 mg, 0.6 mmol,) was reacted with (2-chlorophenyl)acetic acid (132 mg, 0.8 mmol). Pure 2-(2-chlorophenyl)-N-[4-(cyclobutyloxy)-3-sulfamoylphenyl]acetamide was obtained after preparative HPLC (Waters XBrigde C18 5μ 100×30 mm, methanol/water+0.1% formic acid) (137 mg, 0.34 mmol, 54% yield, 99% purity).

LC-MS (Method H): Rt=1.22 min

MS (ESIpos): m/z=394 (M+H)⁺

¹H-NMR (DMSO-d₆) δ [ppm]: 1.51-1.70 (m, 1H), 1.70-1.86 (m, 1H), 2.07-2.29 (m, 2H), 2.31-2.46 (m, 2H), 3.79 (s, 2H), 4.70-4.86 (m, 1H), 6.85-7.06 (m, 3H), 7.23-7.35 (m, 2H), 7.37-7.51 (m, 2H), 7.72 (dd, 1H), 8.01 (d, 1H), 10.29 (s, 1H).

### Example 206

### 2-Phenyl-N-[3-sulfamoyl-4-(tetrahydro-2H-pyran-4-yloxy)phenyl]acetamide

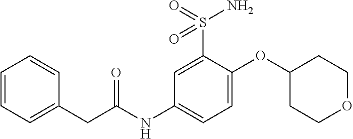 [SMILES: NS(=O)(=O)c1cc(NC(=O)Cc2ccccc2)ccc1OC1CCOCC1]

According to GP3.2 5-amino-2-(tetrahydro-2H-pyran-4-yloxy)benzenesulfonamide (140 mg, 0.5 mmol) was reacted with phenylacetic acid (84 mg, 0.6 mmol). Pure 2-phenyl-N-[3-sulfamoyl-4-(tetrahydro-2H-pyran-4-yloxy)phenyl]acetamide was obtained after preparative HPLC (Waters XBrigde C18 5μ 150×50 mm, acetonitrile/water+0.1% formic acid) (58 mg, 0.15 mmol, 29% yield, 99% purity)

LC-MS (Method A): Rt=0.89 min

MS (ESIpos): m/z=390 (M+H)⁺

¹H-NMR (DMSO-d₆) δ [ppm]: 1.63-1.85 (m, 2H), 1.85-1.99 (m, 2H), 3.40-3.53 (m, 2H), 3.61 (s, 2H), 3.77-3.97 (m, 2H), 4.63-4.81 (m, 1H), 6.86 (s, 2H), 7.16-7.43 (m, 6H), 7.76 (dd, 1H), 8.02 (d, 1H), 10.26 (s, 1H).

### Example 207

### 2-(2-Chlorophenyl)-N-[3-sulfamoyl-4-(tetrahydro-2H-pyran-4-yloxy)phenyl]acetamide

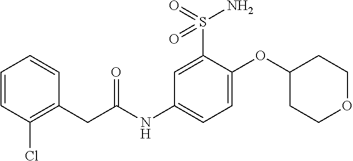 [SMILES: NS(=O)(=O)c1cc(NC(=O)Cc2ccccc2Cl)ccc1OC1CCOCC1]

According to GP3.2 5-amino-2-(tetrahydro-2H-pyran-4-yloxy)benzenesulfonamide (140 mg, 0.5 mmol,) was reacted with (2-chlorophenyl)acetic acid (105 mg, 0.6 mmol). Pure 2-(2-chlorophenyl)-N-[3-sulfamoyl-4-(tetrahydro-2H-pyran-4-yloxy)phenyl]acetamide was obtained after preparative HPLC (Waters XBrigde C18 5μ 150×50 mm, acetonitrile/water+0.1% formic acid) (115 mg, 0.27 mmol, 53% yield, 99% purity).

LC-MS (Method A): Rt=0.96 min

MS (ESIpos): m/z=424 (M+H)⁺

¹H-NMR (DMSO-d₆) δ [ppm]: 1.68-1.86 (m, 2H), 1.87-2.00 (m, 2H), 3.39-3.54 (m, 2H), 3.72-3.95 (m, 4H), 4.63-4.81 (m, 1H), 6.87 (s, 2H), 7.17-7.36 (m, 3H), 7.37-7.52 (m, 2H), 7.75 (dd, 1H), 8.04 (d, 1H), 10.31 (s, 1H).

### Example 208

### 3-(2-{[4-(3-Chlorophenoxy)-3-sulfamoylphenyl]amino}-2-oxoethyl)-N-(2-methoxyethyl)-N-methylbenzamide

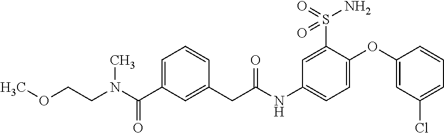 [SMILES: COCCN(C)C(=O)c1cccc(CC(=O)Nc2ccc(Oc3cccc(Cl)c3)c(S(N)(=O)=O)c2)c1]

According to GP3.2 and GP4 5-amino-2-(3-chlorophenoxy)-N-(2,4-dimethoxybenzyl)benzenesulfonamide (150 mg, 0.334 mmol) and {3-[(2-methoxyethyl)(methyl)carbamoyl]phenyl}acetic acid (101 mg, 0.401 mmol) were reacted to 3-(2-{[4-(3-chlorophenoxy)-3-sulfamoylphenyl]amino}-2-oxoethyl)-N-(2-methoxyethyl)-N-methylbenzamide. The pure compound was obtained after preparative HPLC (Waters XBrigde C18 5μ 100×30 mm, acetonitrile/water+0.2% aqueous ammonia (32%)) followed by preparative HPLC (Waters XBrigde C18 5μ 100×30 mm, acetonitrile/water+0.1% formic acid) (1.5 mg, 0.003 mmol, 0.8% yield over 2 steps, 95% yield).

LC-MS (Method A): Rt=1.05 min

MS (ESIpos): m/z=532 (M+H)⁺

### Example 209

### N-[4-(3-Chlorophenoxy)-3-sulfamoylphenyl]-2-(5-chloropyridin-2-yl)acetamide

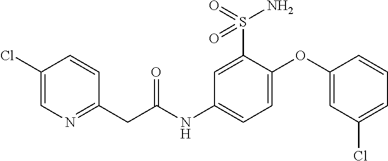 [SMILES: NS(=O)(=O)c1cc(NC(=O)Cc2ccc(Cl)cn2)ccc1Oc1cccc(Cl)c1]

According to GP3.2 and GP4 5-amino-2-(3-chlorophenoxy)-N-(2,4-dimethoxybenzyl)benzenesulfonamide (150 mg, 0.334 mmol) and (5-chloropyridin-2-yl)acetic acid (67 mg, 0.401 mmol) were reacted to N-[4-(3-chlorophenoxy)-3-sulfamoylphenyl]-2-(5-chloropyridin-2-yl)acetamide. The pure compound was obtained after preparative HPLC (Waters XBrigde C18 5μ 100×30 mm, acetonitrile/water+0.2% aqueous ammonia (32%)) (52 mg, 0.115 mmol, 30% yield over 2 steps, 95% purity).

LC-MS (Method E): Rt=1.05 min

MS (ESIneg): m/z=450 (M−H)⁺

¹H-NMR (DMSO-d₆) δ [ppm]: 3.87 (s, 2H), 6.93-7.03 (m, 1H), 7.05-7.13 (m, 2H), 7.16-7.24 (m, 1H), 7.34-7.44 (m, 3H), 7.44-7.50 (m, 1H), 7.80 (dd, 1H), 7.91 (dd, 1H), 8.21 (d, 1H), 8.56 (d, 1H), 10.54 (s, 1H).

### Example 210

### N-[4-(3-Chlorophenoxy)-3-sulfamoylphenyl]-2-[3-(2-methoxyethoxy)phenyl]acetamide

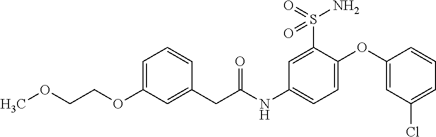 [SMILES: COCCOc1cccc(CC(=O)Nc2ccc(Oc3cccc(Cl)c3)c(S(N)(=O)=O)c2)c1]

According to GP3.2 and GP4 5-amino-2-(3-chlorophenoxy)-N-(2,4-dimethoxybenzyl)benzenesulfonamide (150 mg, 0.334 mmol) and [3-(2-methoxyethoxy)phenyl]acetic acid (84 mg, 0.401 mmol) were reacted to N-[4-(3-chlorophenoxy)-3-sulfamoylphenyl]-2-[3-(2-methoxyethoxy)phenyl]acetamide. The pure compound was obtained after preparative HPLC (Waters XBrigde C18 5μ 100×30 mm, acetonitrile/water+0.2% aqueous ammonia (32%)) (13 mg, 0.025 mmol, 7% yield over 2 steps, 95% purity).

LC-MS (Method E): Rt=1.10 min

MS (ESIneg): m/z=489 (M−H)⁺

¹H-NMR (DMSO-d₆) δ [ppm]: 3.30 (s, 3H), 3.55-3.71 (m, 4H), 4.00-4.16 (m, 2H), 6.78-6.87 (m, 1H), 6.87-6.93 (m, 2H), 6.93-7.00 (m, 1H), 7.06-7.12 (m, 2H), 7.15-7.29 (m, 2H), 7.36-7.48 (m, 3H), 7.81 (dd, 1H), 8.19 (d, 1H), 10.45 (s, 1H).

### Example 211

### N-[4-(3-Chlorophenoxy)-3-sulfamoylphenyl]-2-[2-(2-methoxyethoxy)phenyl]acetamide

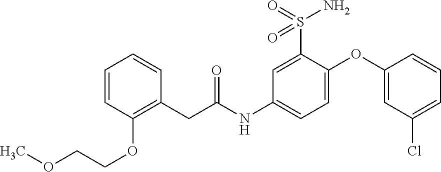 [SMILES: COCCOc1ccccc1CC(=O)Nc1ccc(Oc2cccc(Cl)c2)c(S(N)(=O)=O)c1]

According to GP3.2 and GP4 5-amino-2-(3-chlorophenoxy)-N-(2,4-dimethoxybenzyl)benzenesulfonamide (170 mg, 0.379 mmol) and [2-(2-methoxyethoxy)phenyl]acetic acid (159 mg, 0.454 mmol, 60% pure) were reacted to N-[4-(3-chlorophenoxy)-3-sulfamoylphenyl]-2-[2-(2-methoxyethoxy)phenyl]acetamide. The pure compound was obtained after preparative HPLC (Waters XBrigde C18 5μ 100×30 mm, acetonitrile/water+0.2% aqueous ammonia (32%)) (125 mg, 0.254 mmol, 67% yield over 2 steps, 95% purity).

LC-MS (Method E): Rt=1.14 min

MS (ESIneg): m/z=489 (M−H)⁺

¹H-NMR (DMSO-d₆) δ [ppm]: 3.22 (s, 3H), 3.56-3.72 (m, 4H), 4.03-4.13 (m, 2H), 6.86-6.95 (m, 1H), 6.95-7.04 (m, 2H), 7.05-7.14 (m, 2H), 7.17-7.27 (m, 3H), 7.32-7.47 (m, 3H), 7.81 (dd, 1H), 8.23 (d, 1H), 10.31 (s, 1H).

### Example 212

### N-[4-(3-Chlorophenoxy)-3-sulfamoylphenyl]-2-[3-(2-hydroxyethoxy)phenyl]acetamide

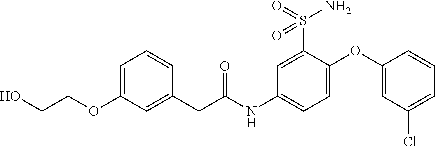 [SMILES: NS(=O)(=O)c1cc(NC(=O)Cc2cccc(OCCO)c2)ccc1Oc1cccc(Cl)c1]

According to GP3.2 and GP4 5-amino-2-(3-chlorophenoxy)-N-(2,4-dimethoxybenzyl)benzenesulfonamide (150 mg, 0.334 mmol) and [3-(2-tert-butoxyethoxy)phenyl]acetic acid (169 mg, 0.401 mmol, 60% purity) were reacted to N-[4-(3-chlorophenoxy)-3-sulfamoylphenyl]-2-[3-(2-hydroxyethoxy)phenyl]acetamide. The pure compound was obtained after preparative HPLC (Waters XBrigde C18 5μ 100×30 mm, acetonitrile/water+0.2% aqueous ammonia (32%)) (18 mg, 0.040 mmol, 11% yield over 2 steps, 95% purity).

LC-MS (Method E): Rt=1.06 min

MS (ESIneg): m/z=475 (M−H)⁺

¹H-NMR (DMSO-d₆) δ [ppm]: 3.60 (s, 2H), 3.67-3.74 (m, 1H), 3.97 (t, 2H), 4.85 (t, 1H), 6.80-6.85 (m, 1H), 6.87-6.94 (m, 2H), 6.95-6.99 (m, 1H), 7.05-7.11 (m, 2H), 7.16-7.28 (m, 2H), 7.30-7.43 (m, 3H), 7.81 (dd, 1H), 8.19 (d, 1H), 10.44 (s, 1H).

### Example 213

### N-[4-(3-Chlorophenoxy)-3-sulfamoylphenyl]-2-[2-(2-hydroxyethoxy)phenyl]acetamide

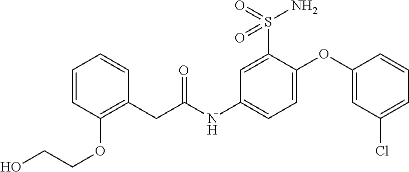 [SMILES: NS(=O)(=O)c1cc(NC(=O)Cc2ccccc2OCCO)ccc1Oc1cccc(Cl)c1]

According to GP3.2 and GP4 5-amino-2-(3-chlorophenoxy)-N-(2,4-dimethoxybenzyl)benzenesulfonamide (170 mg, 0.379 mmol) and [2-(2-tert-butoxyethoxy)phenyl]acetic acid (229 mg, 0.454 mmol, 50% purity) were reacted to N-[4-(3-chlorophenoxy)-3-sulfamoylphenyl]-2-[2-(2-hydroxyethoxy)phenyl]acetamide. The pure compound was obtained after preparative HPLC (Waters XBrigde C18 5μ 100×30 mm, acetonitrile/water+0.2% aqueous ammonia (32%)) (42 mg, 0.090 mmol, 23% yield over 2 steps, 95% purity).

LC-MS (Method E): Rt=1.02 min

MS (ESIneg): m/z=475 (M−H)⁺

¹H-NMR (DMSO-d₆) δ [ppm]: 3.63-3.74 (m, 4H), 4.00 (t, 2H), 4.82 (t, 1H), 6.88-6.95 (m, 1H), 6.96-7.02 (m, 2H), 7.06-7.14 (m, 2H), 7.16-7.27 (m, 3H), 7.36-7.44 (m, 3H), 7.80 (dd, 1H), 8.22 (d, 1H), 10.29 (s, 1H).

### Example 214

### N-[4-(3-Chlorophenoxy)-3-sulfamoylphenyl]-2-(2-fluorophenyl)acetamide

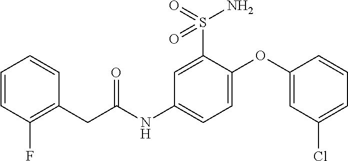 [SMILES: NS(=O)(=O)c1cc(NC(=O)Cc2ccccc2F)ccc1Oc1cccc(Cl)c1]

According to GP3.1 and GP4 5-amino-2-(3-chlorophenoxy)-N-(2,4-dimethoxybenzyl)benzenesulfonamide (300 mg, 0.668 mmol) and (2-fluorophenyl)acetic acid (124 mg, 0.802 mmol) were reacted to N-[4-(3-chlorophenoxy)-3-sulfamoylphenyl]-2-(2-fluorophenyl)acetamide. The pure compound was obtained after preparative HPLC (Chromatorex C-18 10 μm, 125×30 mm, acetonitrile/water+0.1% formic acid). (246 mg, 0.56 mmol, 85% yield, 95% purity).

LC-MS (Method A): Rt=1.18 min

MS (ESIneg): m/z=433 (M−H)⁺

¹H-NMR (DMSO-d₆) δ [ppm]: 3.74 (s, 2H), 6.94-7.01 (m, 1H), 7.05-7.10 (m, 2H), 7.14-7.22 (m, 4H), 7.29-7.42 (m, 4H), 7.79 (dd, 1H), 8.20 (d, 1H), 10.52 (s, 1H).

### Example 215

### N-[4-(Oxetan-3-yloxy)-3-sulfamoylphenyl]-2-phenylacetamide

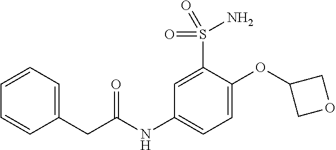 [SMILES: NS(=O)(=O)c1cc(NC(=O)Cc2ccccc2)ccc1OC1COC1]

According to GP3.2 and GP4 5-amino-N-(2,4-dimethoxybenzyl)-2-(oxetan-3-yloxy)benzenesulfonamide (100 mg, 0.3 mmol) were converted with phenylacetic acid (41 mg, 0.3 mmol) to N-[4-(oxetan-3-yloxy)-3-sulfamoylphenyl]-2-phenylacetamide. The pure compound was obtained after preparative HPLC (Waters XBrigde C18 5μ 100×30 mm, acetonitrile/water+0.1% trifluoroacetic acid) (14 mg, 0.040 mmol, 4% yield over 2 steps, 94% purity).

LC-MS (Method A): Rt=0.81 min

MS (ESIpos): m/z=362 (M+H)⁺

¹H-NMR (DMSO-d₆) δ [ppm]: 3.81 (s, 2H), 4.65-4.75 (m, 2H), 4.89 (t, 2H), 5.28-5.41 (m, 1H), 6.78 (d, 1H), 7.12 (s, 2H), 7.29-7.32 (m, 2H), 7.39-7.46 (m, 3H), 7.73 (dd, 1H), 8.06 (d, 1H), 10.34 (s, 1H).

### Example 216

### 2-(2-Chlorophenyl)-N-[4-(oxetan-3-yloxy)-3-sulfamoylphenyl]acetamide

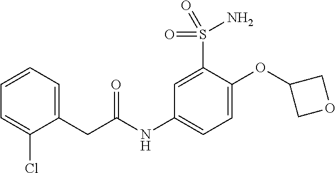 [SMILES: NS(=O)(=O)c1cc(NC(=O)Cc2ccccc2Cl)ccc1OC1COC1]

According to GP3.2 and GP4 5-amino-N-(2,4-dimethoxybenzyl)-2-(oxetan-3-yloxy)benzenesulfonamide (100 mg, 0.3 mmol) were converted with (2-chlorophenyl)acetic acid (52 mg, 0.3 mmol) to 2-(2-chlorophenyl)-N-[4-(oxetan-3-yloxy)-3-sulfamoylphenyl]acetamide. The pure compound was obtained after preparative HPLC (Waters XBrigde C18 5μ 100×30 mm, acetonitrile/water+0.1% trifluoroacetic acid) (7 mg, 0.020 mmol, 2% yield over 2 steps, 90% purity).

LC-MS (Method F): Rt=0.86 min

MS (ESIpos): m/z=396 (M+H)⁺

¹H-NMR (DMSO-d₆) δ [ppm]: 3.61 (s, 2H), 4.69 (dd, 2H), 4.88 (t, 2H), 5.28-5.39 (m, 1H), 6.76 (d, 1H), 7.12 (s, 2H), 7.20-7.29 (m, 1H), 7.31-7.33 (m, 3H), 7.74 (dd, 1H), 8.05 (d, 1H), 10.28 (s, 1H).

### Example 217

### N-[4-(Cyclopentyloxy)-3-sulfamoylphenyl]-2-phenylacetamide

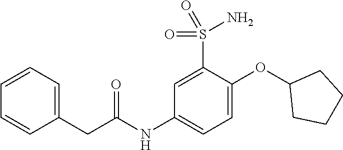 [SMILES: NS(=O)(=O)c1cc(NC(=O)Cc2ccccc2)ccc1OC1CCCC1]

According to GP3.2 and GP4 5-amino-2-(cyclopentyloxy)-N-(2,4-dimethoxybenzyl)benzenesulfonamide (75 mg, 0.2 mmol) was converted with phenylacetic acid (30 mg, 0.2 mmol) to N-[4-(cyclopentyloxy)-3-sulfamoylphenyl]-2-phenylacetamide. The pure compound was obtained after preparative HPLC (Waters XBrigde C18 5μ 100×30 mm, acetonitrile/water+0.1% formic acid) (14 mg, 0.040 mmol, 6% yield, 96% purity).

LC-MS (Method D): Rt=1.07 min

MS (ESIneg): m/z=373 (M−H)⁺

¹H-NMR (DMSO-d₆) δ [ppm]: 1.47-1.63 (m, 2H), 1.67-1.79 (m, 2H), 1.85 (d, 4H), 3.60 (s, 2H), 4.90-5.00 (m, 1H), 6.75 (s, 2H), 7.16 (s, 1H), 7.20-7.29 (m, 1H), 7.32 (d, 4H), 7.71-7.80 (m, 1H), 8.00 (d, 1H), 10.23 (s, 1H).

### Example 218

### 2-(2-Chlorophenyl)-N-[4-(cyclopentyloxy)-3-sulfamoylphenyl]acetamide

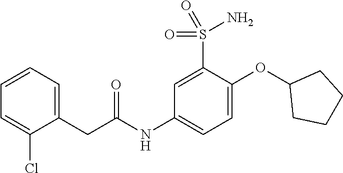 [SMILES: NS(=O)(=O)c1cc(NC(=O)Cc2ccccc2Cl)ccc1OC1CCCC1]

According to GP3.2 and GP4 5-amino-2-(cyclopentyloxy)-N-(2,4-dimethoxybenzyl)benzenesulfonamide (75 mg, 0.2 mmol) was converted with (2-chlorophenyl)acetic acid (38 mg, 0.2 mmol) to 2-(2-chlorophenyl)-N-[4-(cyclopentyloxy)-3-sulfamoylphenyl]acetamide. The pure compound was obtained after preparative HPLC (Waters XBrigde C18 5μ 100×30 mm, acetonitrile/water+0.1% formic acid) (11 mg, 0.030 mmol, 3% yield, 97% purity).

LC-MS (Method D): Rt=1.13 min

MS (ESIneg): m/z=407 (M−H)⁺

¹H-NMR (DMSO-d₆) δ [ppm]: 1.47-1.62 (m, 2H), 1.68-1.81 (m, 2H), 1.85 (br. s., 4H), 3.80 (s, 2H), 4.91-5.01 (m, 1H), 6.76 (s, 2H), 7.11-7.19 (m, 1H), 7.31 (s, 2H), 7.39-7.49 (m, 2H), 7.70-7.81 (m, 1H), 8.02 (d, 1H), 10.25-10.33 (m, 1H).

### Example 219

### N-{4-[(1-Methylpiperidin-3-yl)oxy]-3-sulfamoylphenyl}-2-phenylacetamide

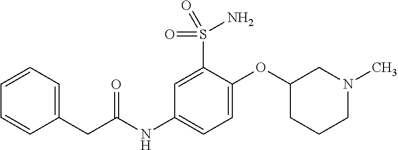 [SMILES: CN1CCCC(Oc2ccc(NC(=O)Cc3ccccc3)cc2S(N)(=O)=O)C1]

According to GP3.2 and GP4 to 5-amino-N-(2,4-dimethoxybenzyl)-2-{[(3R)-1-methylpiperidin-3-yl]oxy}benzenesulfonamide (90 mg, 0.2 mmol) was converted with phenylacetic acid (34 mg, 0.2 mmol) to N-{4-[(1-methylpiperidin-3-yl)oxy]-3-sulfamoylphenyl}-2-phenylacetamide. The pure compound was obtained after preparative HPLC (Waters XBrigde C18 5μ 100×30 mm, acetonitrile/water+0.1% formic acid) (9.2 mg, 0.020 mmol, 3% yield, 97% purity).

LC-MS (Method D): Rt=0.71 min

MS (ESIneg): m/z=402 (M−H)⁺

¹H-NMR (CD₃OD) δ [ppm]: 1.65-1.89 (m, 2H), 2.03-2.27 (m, 2H), 2.41-2.62 (m, 3H), 2.65-2.97 (m, 3H), 3.03-3.27 (m, 2H), 3.67 (s, 2H), 7.23-7.28 (m, 2H), 7.34 (s, 4H), 7.77-7.85 (m, 1H), 8.04-8.11 (m, 1H).

### Example 220

### 2-(2-Chlorophenyl)-N-{4-[(1-methylpiperidin-3-yl)oxy]-3-sulfamoylphenyl}acetamide

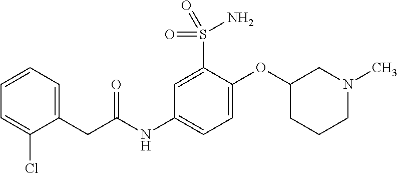 [SMILES: CN1CCCC(Oc2ccc(NC(=O)Cc3ccccc3Cl)cc2S(N)(=O)=O)C1]

According to GP3.2 and GP4 to 5-amino-N-(2,4-dimethoxybenzyl)-2-{[(3R)-1-methylpiperidin-3-yl]oxy}benzenesulfonamide (90 mg, 0.2 mmol) was converted with (2-chlorophenyl)acetic acid (42 mg, 0.2 mmol) to 2-(2-chlorophenyl)-N-{4-[(1-methylpiperidin-3-yl)oxy]-3-sulfamoylphenyl}acetamide. The pure compound was obtained after preparative HPLC (Waters XBrigde C18 5μ 100×30 mm, acetonitrile/water+0.1% formic acid) (16 mg, 0.040 mmol, 5% yield, 97% purity).

LC-MS (Method D): Rt=0.77 min

MS (ESIneg): m/z=436 (M−H)⁺

¹H-NMR (CD₃OD) δ [ppm]: 1.64-1.88 (m, 2H), 2.05-2.22 (m, 2H), 2.37-2.64 (m, 4H), 2.70-2.89 (m, 1H), 2.95-3.18 (m, 2H), 3.87 (s, 2H), 4.88-4.97 (m, 1H), 7.23-7.34 (m, 3H), 7.38-7.44 (m, 2H), 7.77-7.86 (m, 1H), 8.02-8.12 (m, 1H).

### Example 221

### N-{4-[(1-Methylpyrrolidin-3-yl)oxy]-3-sulfamoylphenyl}-2-phenylacetamide

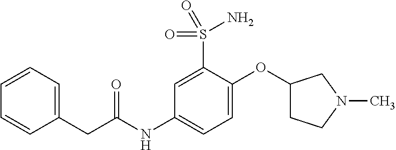 [SMILES: CN1CCC(Oc2ccc(NC(=O)Cc3ccccc3)cc2S(N)(=O)=O)C1]

According to GP3.2 and GP4 5-amino-2-(cyclopentyloxy)-N-(2,4-dimethoxybenzyl)benzenesulfonamide (90 mg, 0.2 mmol) was converted with phenylacetic acid (34 mg, 0.3 mmol) to N-{4-[(1-methylpyrrolidin-3-yl)oxy]-3-sulfamoylphenyl}-2-phenylacetamide. The pure compound was obtained after preparative HPLC (Waters XBrigde C18 5μ 100×30 mm, acetonitrile/water+0.1% formic acid) (7 mg, 0.020 mmol, 2% yield, 93% purity).

LC-MS (Method D): Rt=0.68 min

MS (ESIneg): m/z=388 (M−H)⁺

¹H-NMR (CD₃OD) δ [ppm]: 2.27-2.41 (m, 1H), 2.41-2.56 (m, 1H), 2.87 (s, 3H), 3.11-3.24 (m, 1H), 3.35 (s, 1H), 3.52-3.64 (m, 2H), 3.67 (s, 2H), 5.27-5.35 (m, 1H), 7.17 (d, 1H), 7.26 (d, 1H), 7.29-7.38 (m, 4H), 7.82 (dd, 1H), 8.08 (d, 1H).

### Example 222

### 2-(2-Chlorophenyl)-N-{4-[(1-methylpyrrolidin-3-yl)oxy]-3-sulfamoylphenyl}acetamide

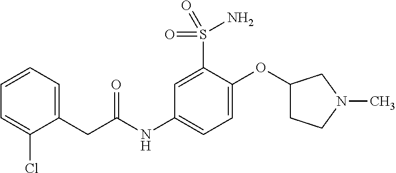 [SMILES: CN1CCC(Oc2ccc(NC(=O)Cc3ccccc3Cl)cc2S(N)(=O)=O)C1]

According to GP3.2 and GP4 5-amino-2-(cyclopentyloxy)-N-(2,4-dimethoxybenzyl)benzenesulfonamide (90 mg, 0.2 mmol) was converted with (2-chlorophenyl)acetic acid (44 mg, 0.3 mmol) to 2-(2-chlorophenyl)-N-{4-[(1-methylpyrrolidin-3-yl)oxy]-3-sulfamoylphenyl}acetamide. The pure compound was obtained after preparative HPLC (Waters XBrigde C18 5μ 100×30 mm, acetonitrile/water+0.1% formic acid) (9 mg, 0.020 mmol, 3% yield, 97% purity).

LC-MS (Method D): Rt=0.75 min

MS (ESIneg): m/z=422 (M−H)⁺

¹H-NMR (CD₃OD) δ [ppm]: 2.25-2.37 (m, 1H), 2.41-2.55 (m, 1H), 2.79 (s, 3H), 2.95-3.08 (m, 1H), 3.16-3.26 (m, 1H), 3.43-3.58 (m, 2H), 3.87 (s, 2H), 5.24-5.32 (m, 1H), 7.14-7.21 (m, 1H), 7.27-7.35 (m, 2H), 7.36-7.44 (m, 2H), 7.77-7.87 (m, 1H), 8.03-8.11 (m, 1H).

### Example 223

### N-[4-(4-Chlorophenoxy)-3-sulfamoylphenyl]-2-(4-fluorophenyl)acetamide

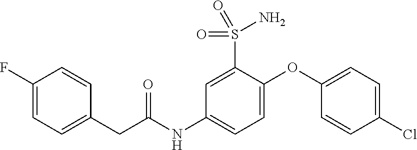 [SMILES: NS(=O)(=O)c1cc(NC(=O)Cc2ccc(F)cc2)ccc1Oc1ccc(Cl)cc1]

According to GP3.1 and GP4 5-amino-2-(4-chlorophenoxy)-N-(2,4-dimethoxybenzyl)benzenesulfonamide (150 mg, 0.334 mmol) was reacted with (4-fluorophenyl)acetic acid (77 mg, 0.501 mmol) to N-[4-(4-chlorophenoxy)-3-sulfamoylphenyl]-2-(4-fluorophenyl)acetamide. The pure compound was obtained after preparative HPLC (Waters XBrigde C18 5μ 100×30 mm, acetonitrile/water+0.2% aqueous ammonia (32%)) (16 mg, 0.036 mmol, 11% yield, 95% purity).

LC-MS (Method E): Rt=1.12 min

MS (ESIneg): m/z=433 (M−H)⁺

¹H-NMR (DMSO-d₆) δ [ppm]: 3.65 (s, 2H), 6.99-7.07 (m, 3H), 7.13-7.20 (m, 2H), 7.34-7.40 (m, 4H), 7.41-7.46 (m, 2H), 7.78 (dd, 1H), 8.18 (d, 1H), 10.45 (s, 1H).

### Example 224

### N-[4-(3-Chlorophenoxy)-3-sulfamoylphenyl]-2-(4-cyanophenyl)acetamide

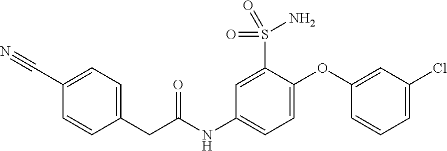 [SMILES: N#Cc1ccc(CC(=O)Nc2ccc(Oc3cccc(Cl)c3)c(S(N)(=O)=O)c2)cc1]

According to GP3.1 and GP4 5-amino-2-(3-chlorophenoxy)-N-(2,4-dimethoxybenzyl)benzenesulfonamide (75 mg, 0.167 mmol) was reacted with (4-cyanophenyl)acetic acid (33 mg, 0.200 mmol) to N-[4-(3-chlorophenoxy)-3-sulfamoylphenyl]-2-(4-cyanophenyl)acetamide. The pure compound was obtained after preparative HPLC (Waters XBrigde C18 5μ 100×30 mm, acetonitrile/water+0.1% formic acid) (6.0 mg, 0.013 mmol, 8% yield over 2 steps, 95% purity).

LC-MS (Method A): Rt=1.10 min

MS (ESIpos): m/z=442 (M+H)⁺

¹H-NMR (DMSO-d₆) δ [ppm]: 3.79 (s, 2H), 6.93-7.02 (m, 1H), 7.06-7.12 (m, 2H), 7.17-7.23 (m, 1H), 7.35-7.45 (m, 3H), 7.54 (d, 2H), 7.81 (d, 3H), 8.18 (d, 1H), 10.54 (s, 1H).

### Example 225

### N-[4-(3-Chlorophenoxy)-3-sulfamoylphenyl]-2-(2-cyanophenyl)acetamide

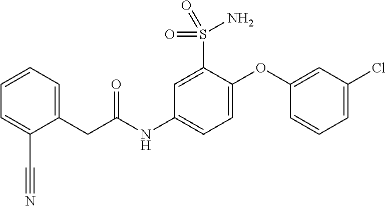 [SMILES: N#Cc1ccccc1CC(=O)Nc1ccc(Oc2cccc(Cl)c2)c(S(N)(=O)=O)c1]

According to GP3.1 and GP4 5-amino-2-(3-chlorophenoxy)-N-(2,4-dimethoxybenzyl)benzenesulfonamide (75 mg, 0.167 mmol) was reacted with (2-cyanophenyl)acetic acid (32 mg, 0.200 mmol) to N-[4-(3-chlorophenoxy)-3-sulfamoylphenyl]-2-(2-cyanophenyl)acetamide. The pure compound was obtained after preparative HPLC (Waters XBrigde C18 5μ 100×30 mm, acetonitrile/water+0.2% aqueous ammonia (32%)) (11 mg, 0.024 mmol, 15% yield over 2 steps, 95% purity).

LC-MS (Method B): Rt=1.06 min

MS (ESIpos): m/z=442 (M+H)⁺

¹H-NMR (DMSO-d₆) δ [ppm]: 3.96 (s, 2H), 6.92-7.02 (m, 1H), 7.05-7.13 (m, 2H), 7.17-7.23 (m, 1H), 7.34-7.43 (m, 3H), 7.48 (d, 1H), 7.58 (s, 1H), 7.69 (d, 1H), 7.76-7.87 (m, 2H), 8.21 (d, 1H), 10.61 (s, 1H).

### Example 226

### N-[4-(3-Chlorophenoxy)-3-sulfamoylphenyl]-2-(3-cyanophenyl)acetamide

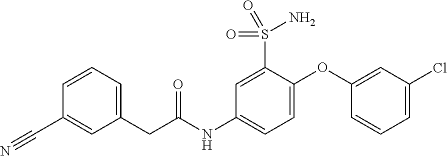 [SMILES: N#Cc1cccc(CC(=O)Nc2ccc(Oc3cccc(Cl)c3)c(S(N)(=O)=O)c2)c1]

According to GP3.1 and GP4 5-amino-2-(3-chlorophenoxy)-N-(2,4-dimethoxybenzyl)benzenesulfonamide (75 mg, 0.167 mmol) and (3-cyanophenyl)acetic acid (34 mg, 0.200 mmol) were converted to N-[4-(3-chlorophenoxy)-3-sulfamoylphenyl]-2-(3-cyanophenyl)acetamide. The pure compound was obtained after preparative HPLC (Waters XBrigde C18 5μ 100×30 mm, acetonitrile/water+0.1% formic acid) (18 mg, 0.039 mmol, 23% yield over 2 steps, 95% purity).

LC-MS (Method A): Rt=1.11 min

MS (ESIneg): m/z=440 (M−H)⁺

¹H-NMR (DMSO-d₆) δ [ppm]: 3.76 (s, 2H), 6.92-7.02 (m, 1H), 7.04-7.13 (m, 2H), 7.16-7.24 (m, 1H), 7.34-7.44 (m, 3H), 7.56 (s, 1H), 7.79 (d, 4H), 8.19 (d, 1H), 10.50 (s, 1H).

### Example 227

### N-[4-(4-Chlorophenoxy)-3-sulfamoylphenyl]-2-[4-(trifluoromethyl)phenyl]acetamide

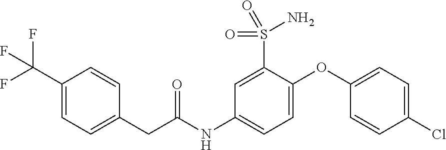 [SMILES: NS(=O)(=O)c1cc(NC(=O)Cc2ccc(C(F)(F)F)cc2)ccc1Oc1ccc(Cl)cc1]

According to GP3.1 and GP4 5-amino-2-(4-chlorophenoxy)-N-(2,4-dimethoxybenzyl)benzenesulfonamide (150 mg, 0.334 mmol) was reacted with [4-(trifluoromethyl)phenyl]acetic acid (102 mg, 0.401 mmol) to N-[4-(4-chlorophenoxy)-3-sulfamoylphenyl]-2-[4-(trifluoromethyl)phenyl]acetamide. The pure compound was obtained after preparative HPLC (Waters XBrigde C18 5μ 100×30 mm, acetonitrile/water+0.2% aqueous ammonia (32%)) (24 mg, 0.049 mmol, 15% yield over 2 steps, 95% purity).

LC-MS (Method E): Rt=1.24 min

MS (ESIneg): m/z=483 (M−H)⁺

¹H-NMR (DMSO-d₆) δ [ppm]: 3.74-3.81 (m, 2H), 6.98-7.07 (m, 3H), 7.34-7.47 (m, 4H), 7.56 (d, 2H), 7.70 (d, 2H), 7.77 (dd, 1H), 8.18 (d, 1H), 10.51 (s, 1H).

### Example 228

### N-[4-(4-Chlorophenoxy)-3-sulfamoylphenyl]-2-(4-chlorophenyl)acetamide

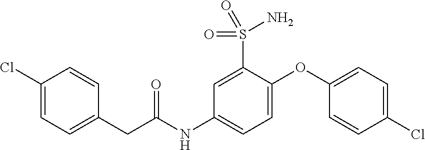 [SMILES: NS(=O)(=O)c1cc(NC(=O)Cc2ccc(Cl)cc2)ccc1Oc1ccc(Cl)cc1]

According to GP3.1 and GP4 5-amino-2-(4-chlorophenoxy)-N-(2,4-dimethoxybenzyl)benzenesulfonamide (150 mg, 0.334 mmol) was reacted with (4-chlorophenyl)acetic acid (85 mg, 0.501 mmol) to N-[4-(4-chlorophenoxy)-3-sulfamoylphenyl]-2-(4-chlorophenyl)acetamide. The pure compound was obtained preparative HPLC (Waters XBrigde C18 5μ 100×30 mm, acetonitrile/water+0.2% aqueous ammonia (32%)) (13 mg, 0.028 mmol, 8% yield over 2 steps, 95% purity).

LC-MS (Method E): Rt=1.20 min

MS (ESIneg): m/z=449 (M−H)⁺

¹H-NMR (DMSO-d₆) δ [ppm]: 3.66 (s, 2H), 6.98-7.10 (m, 3H), 7.34-7.46 (m, 8H), 7.78 (dd, 1H), 8.18 (d, 1H), 10.46 (s, 1H).

### Example 229

### N-[4-(4-Chlorophenoxy)-3-sulfamoylphenyl]-2-(4-methoxyphenyl)acetamide

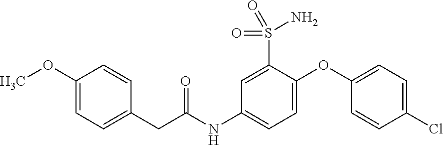 [SMILES: COc1ccc(CC(=O)Nc2ccc(Oc3ccc(Cl)cc3)c(S(N)(=O)=O)c2)cc1]

According to GP3.1 and GP4 5-amino-2-(4-chlorophenoxy)-N-(2,4-dimethoxybenzyl)benzenesulfonamide (150 mg, 0.334 mmol) was reacted with (4-methoxyphenyl)acetic acid (83 mg, 0.501 mmol) to N-[4-(4-chlorophenoxy)-3-sulfamoylphenyl]-2-(4-methoxyphenyl)acetamide. The pure compound was obtained after preparative HPLC (Waters XBrigde C18 5μ 100×30 mm, acetonitrile/water+0.2% aqueous ammonia (32%)) (17 mg, 0.038 mmol, 11% yield, 95% purity).

LC-MS (Method E): Rt=1.10 min

MS (ESIneg): m/z=445 (M−H)⁺

¹H-NMR (DMSO-d₆) δ [ppm]: 3.55 (s, 2H), 3.73 (s, 3H), 6.86-6.95 (m, 2H), 6.96-7.07 (m, 3H), 7.20-7.29 (m, 2H), 7.33-7.40 (m, 2H), 7.40-7.47 (m, 2H), 7.78 (dd, 1H), 8.17 (d, 1H), 10.33-10.43 (m, 1H).

### Example 230

### N-[4-(4-Chlorophenoxy)-3-sulfamoylphenyl]-2-(2-fluorophenyl)acetamide

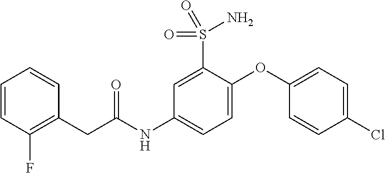 [SMILES: NS(=O)(=O)c1cc(NC(=O)Cc2ccccc2F)ccc1Oc1ccc(Cl)cc1]

According to GP3.1 and GP4 5-amino-2-(4-chlorophenoxy)-N-(2,4-dimethoxybenzyl)benzenesulfonamide (150 mg, 0.334 mmol) was reacted with (2-fluorophenyl)acetic acid (77 mg, 0.501 mmol) to N-[4-(4-chlorophenoxy)-3-sulfamoylphenyl]-2-(2-fluorophenyl)acetamide. The pure compound was obtained after preparative HPLC (Waters XBrigde C18 5μ 100×30 mm, acetonitrile/water+0.2% aqueous ammonia (32%)) (37 mg, 0.086 mmol, 25% yield, 95% purity).

LC-MS (Method E): Rt=1.12 min

MS (ESIneg): m/z=433 (M−H)⁺

¹H-NMR (DMSO-d₆) δ [ppm]: 3.65-3.77 (m, 2H), 6.98-7.06 (m, 3H), 7.13-7.20 (m, 2H), 7.26-7.46 (m, 6H), 7.76 (dd, 1H), 8.18 (d, 1H), 10.46 (s, 1H).

### Example 231

### 2-(2-Chloro-4-fluorophenyl)-N-[4-(4-chlorophenoxy)-3-sulfamoylphenyl]acetamide

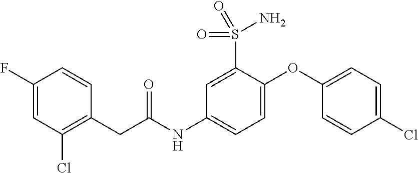 [SMILES: NS(=O)(=O)c1cc(NC(=O)Cc2ccc(F)cc2Cl)ccc1Oc1ccc(Cl)cc1]

According to GP3.1 and GP4 5-amino-2-(4-chlorophenoxy)-N-(2,4-dimethoxybenzyl)benzenesulfonamide (150 mg, 0.334 mmol) was reacted with (2-chloro-4-fluorophenyl)acetic acid (94 mg, 0.501 mmol) to 2-(2-chloro-4-fluorophenyl)-N-[4-(4-chlorophenoxy)-3-sulfamoylphenyl]acetamide. The pure compound was obtained preparative HPLC (Waters XBrigde C18 5μ 100×30 mm, acetonitrile/water+0.2% aqueous ammonia (32%)) (51 mg, 0.109 mmol, 32% yield over 2 steps, 95% purity).

LC-MS (Method E): Rt=1.19 min

MS (ESIneg): m/z=467 (M−H)⁺

¹H-NMR (DMSO-d₆) δ [ppm]: 3.83 (s, 2H), 6.98-7.11 (m, 3H), 7.21 (td, 1H), 7.36 (s, 2H), 7.40-7.51 (m, 4H), 7.76 (dd, 1H), 8.19 (d, 1H), 10.48 (s, 1H).

### Example 232

### 2-(2-Chlorophenyl)-N-{4-[(1,1-dioxidotetrahydrothiophen-3-yl)oxy]-3-sulfamoylphenyl}acetamide

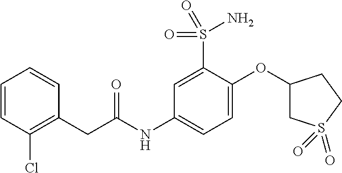 [SMILES: NS(=O)(=O)c1cc(NC(=O)Cc2ccccc2Cl)ccc1OC1CCS(=O)(=O)C1]

According to GP4 2-(2-chlorophenyl)-N-(3-[(2,4-dimethoxybenzyl)sulfamoyl]-4-{[(3S)-1,1-dioxidotetrahydrothiophen-3-yl]oxy}phenyl)acetamide (26 mg, 0.043 mmol) was converted to 2-(2-chlorophenyl)-N-{4-[(1,1-dioxidotetrahydrothiophen-3-yl)oxy]-3-sulfamoylphenyl}acetamide and purified by HPLC (Chromatorex C-18 10 μm, 125×30 mm, acetonitrile/water+0.1% formic acid) (7.3 mg, 0.020 mmol, 37% yield, 97% purity).

LC-MS (Method A): Rt=0.90 min

MS (ESIneg): m/z=457 (M−H)⁺

¹H-NMR (DMSO-d₆) δ [ppm]: 3.17-3.28 (m, 1H), 3.34-3.40 (m, 1H), 3.43-3.53 (m, 1H), 3.55-3.63 (m, 1H), 3.82 (s, 2H), 5.39-5.50 (m, 1H), 6.91-7.03 (m, 2H), 7.21-7.37 (m, 3H), 7.38-7.49 (m, 2H), 7.76-7.86 (m, 1H), 8.02-8.10 (m, 1H), 10.33-10.42 (m, 1H).

### Example 233

### 2-(2-Chlorophenyl)-N-{4-[(1-methyl-1H-pyrazol-4-yl)oxy]-3-sulfamoylphenyl}acetamide

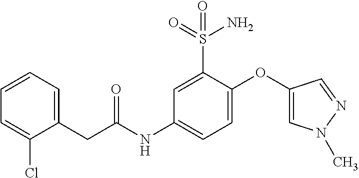 [SMILES: Cn1cc(Oc2ccc(NC(=O)Cc3ccccc3Cl)cc2S(N)(=O)=O)cn1]

According to general procedures GP1.3, GP2.1, GP3.2 and GP4, 2-chloro-N-(2,4-dimethoxybenzyl)-5-nitrobenzenesulfonamide (200 mg, 0.5 mmol), 1-methyl-1H-pyrazol-4-ol (152 mg, 1.6 mmol) and (2-chlorophenyl)acetic acid (127 mg, 0.7 mmol) were converted without purification of intermediates to 2-(2-chlorophenyl)-N-{4-[(1-methyl-1H-pyrazol-4-yl)oxy]-3-sulfamoylphenyl}acetamide. The pure compound was obtained after preparative HPLC (Waters XBrigde C18 5μ 100×30 mm, acetonitrile/water+0.2% aqueous ammonia (32%)) (24 mg, 0.058 mmol, 6% yield over 4 steps, 96% purity).

LC-MS (Method E): Rt=0.83 min

MS (ESIneg): m/z=419 (M−H)⁺

¹H-NMR (DMSO-d₆) δ [ppm]: 3.79-3.84 (m, 5H), 7.04 (d, 1H), 7.28-7.33 (m, 4H), 7.36 (d, 1H), 7.40-7.46 (m, 2H), 7.69-7.75 (m, 2H), 8.12 (d, 1H), 10.40 (s, 1H).

### Example 234

### N-[4-(4-Chlorophenoxy)-3-sulfamoylphenyl]-2-[4-(difluoromethyl)phenyl]acetamide

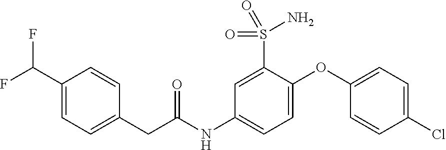 [SMILES: NS(=O)(=O)c1cc(NC(=O)Cc2ccc(C(F)F)cc2)ccc1Oc1ccc(Cl)cc1]

According to GP3.1 and GP4 5-amino-2-(4-chlorophenoxy)-N-(2,4-dimethoxybenzyl)benzenesulfonamide (150 mg, 0.334 mmol) was reacted with ([4-(difluoromethyl)phenyl]acetic acid (93 mg, 0.501 mmol) to N-[4-(4-chlorophenoxy)-3-sulfamoylphenyl]-2-[4-(difluoromethyl)phenyl]acetamide. The pure compound was obtained after preparative HPLC (Waters XBrigde C18 5μ 100×30 mm, acetonitrile/water+0.2% aqueous ammonia (32%)) (15 mg, 0.046 mmol, 15% yield, 98% purity)

LC-MS (Method E): Rt=1.13 min

MS (ESIneg): m/z=465 (M−H)⁺

¹H-NMR (DMSO-d₆) δ [ppm]: 3.69-3.75 (m, 2H), 6.85-7.17 (m, 4H), 7.33-7.39 (m, 2H), 7.41 (s, 4H), 7.51-7.57 (m, 2H), 7.73-7.82 (m, 1H), 8.14-8.20 (m, 1H), 10.49 (s, 1H).

### Example 235

### 2-(2-Chloro-4-methoxyphenyl)-N-[4-(4-chlorophenoxy)-3-sulfamoylphenyl]acetamide

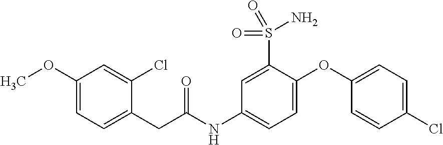 [SMILES: COc1ccc(CC(=O)Nc2ccc(Oc3ccc(Cl)cc3)c(S(N)(=O)=O)c2)c(Cl)c1]

According to GP3.1 and GP4 5-amino-2-(4-chlorophenoxy)-N-(2,4-dimethoxybenzyl)benzenesulfonamide (150 mg, 0.334 mmol) was reacted with (2-chloro-4-methoxyphenyl)acetic acid (106 mg, 0.501 mmol) to 2-(2-chloro-4-methoxyphenyl)-N-[4-(4-chlorophenoxy)-3-sulfamoylphenyl]acetamide. The pure compound was obtained after preparative HPLC (Waters XBrigde C18 5μ 100×30 mm, acetonitrile/water+0.1% trifluoroacetic acid) (19 mg, 0.039 mmol, 10% yield over 2 steps, 95% purity)

LC-MS (Method A): Rt=1.20 min

MS (ESIneg): m/z=479 (M−H)⁺

¹H-NMR (DMSO-d₆) δ [ppm]: 3.58 (s, 2H), 3.83 (s, 3H), 6.96-7.08 (m, 3H), 7.10 (d, 1H), 7.22-7.30 (m, 1H), 7.33-7.48 (m, 5H), 7.73-7.82 (m, 1H), 8.17 (d, 1H), 10.40 (s, 1H).

### Example 236

### 2-(2-Chlorophenyl)-N-{4-[(1-methyl-1H-pyrazol-3-yl)oxy]-3-sulfamoylphenyl}acetamide

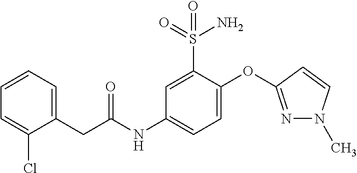 [SMILES: Cn1ccc(Oc2ccc(NC(=O)Cc3ccccc3Cl)cc2S(N)(=O)=O)n1]

According to general procedures GP1.3, GP4, GP2.1 and GP3.2 2-chloro-N-(2,4-dimethoxybenzyl)-5-nitrobenzenesulfonamide (250 mg, 0.6 mmol), 1-methyl-1H-pyrazol-3-ol (190 mg, 1.9 mmol) and (2-chlorophenyl)acetic acid (374 mg, 2.2 mmol) were converted without purification of intermediates to 2-(2-chlorophenyl)-N-{4-[(1-methyl-1H-pyrazol-3-yl)oxy]-3-sulfamoylphenyl}acetamide. The pure compound was obtained after preparative HPLC (Waters XBrigde C18 5μ 100×30 mm, acetonitrile/water+0.2% aqueous ammonia (32%)) (19 mg, 3.6 mmol, 7% yield over 2 steps, 98% purity).

LC-MS (Method G): Rt=1.71 min

MS (ESIneg): m/z=419 (M−H)⁺

¹H-NMR (DMSO-d₆) δ [ppm]: 3.75 (s, 3H), 3.83 (s, 2H), 5.86 (d, 1H), 7.11 (d, 1H), 7.21-7.37 (m, 4H), 7.39-7.50 (m, 2H), 7.64 (d, 1H), 7.69-7.76 (m, 1H), 8.13 (d, 1H), 10.42 (s, 1H).

### Example 237

### 2-(2-Chlorophenyl)-N-{4-[(1-methyl-1H-pyrazol-5-yl)oxy]-3-sulfamoylphenyl}acetamide

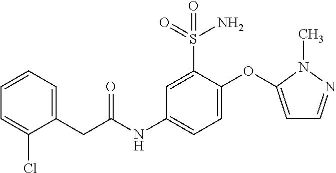 [SMILES: Cn1nccc1Oc1ccc(NC(=O)Cc2ccccc2Cl)cc1S(N)(=O)=O]

According to general procedures GP1.3, GP2.1, GP3.2 and GP4, 2-chloro-N-(2,4-dimethoxybenzyl)-5-nitrobenzenesulfonamide (500 mg, 1.3 mmol), 1-methyl-1H-pyrazol-5-ol (380 mg, 3.9 mmol) and (2-chlorophenyl)acetic acid (176 mg, 1.0 mmol) were converted without purification of intermediates to 2-(2-chlorophenyl)-N-{4-[(1-methyl-1H-pyrazol-5-yl)oxy]-3-sulfamoylphenyl}acetamide. The pure compound was obtained after preparative HPLC (Waters XBrigde C18 5μ 150×50 mm, acetonitrile/water/10% formic acid/acetonitrile) (266 mg, 0.6 mmol, 86% yield over 4 steps, 95% purity).

LC-MS (Method A): Rt=0.92 min

MS (ESIpos): m/z=421 (M+H)⁺

¹H-NMR (DMSO-d₆) δ [ppm]: 3.77-3.86 (m, 5H), 7.04 (d, 1H), 7.23-7.34 (m, 4H), 7.36 (d, 1H), 7.39-7.49 (m, 2H), 7.66-7.77 (m, 2H), 8.12 (d, 1H), 10.39 (s, 1H).

### Example 238

### 2-(2-Chlorophenyl)-N-{4-[(1-methylpiperidin-4-yl)oxy]-3-sulfamoylphenyl}acetamide

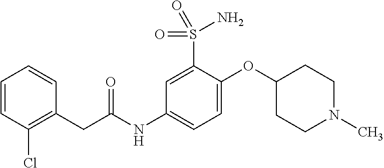 [SMILES: CN1CCC(Oc2ccc(NC(=O)Cc3ccccc3Cl)cc2S(N)(=O)=O)CC1]

According to GP3.2 and GP4 5-amino-N-(2,4-dimethoxybenzyl)-2-[(1-methylpiperidin-4-yl)oxy]benzenesulfonamide (100 mg, 0.2 mmol) was converted with (2-chlorophenyl)acetic acid (47 mg, 0.3 mmol) to 2-(2-chlorophenyl)-N-{4-[(1-methylpiperidin-4-yl)oxy]-3-sulfamoylphenyl}acetamide. The pure compound was obtained after HPLC (Chromatorex C-18 10 μm, 125×30 mm, acetonitrile/water+0.1% formic acid) (42 mg, 0.090 mmol, 40% yield over 2 steps, 97% purity).

LC-MS (Method A): Rt=0.75 min

MS (ESIneg): m/z=436 (M−H)⁺

¹H-NMR (DMSO-d₆) δ [ppm]: 1.89-2.09 (m, 4H), 2.57 (s, 2H), 2.78-2.96 (m, 2H), 3.01-3.14 (m, 3H), 3.80 (s, 2H), 4.71-4.81 (m, 1H), 7.02 (s, 2H), 7.24 (s, 1H), 7.27-7.35 (m, 2H), 7.38-7.46 (m, 2H), 7.71-7.80 (m, 1H), 8.05 (d, 1H), 10.32 (s, 1H).

### Example 239

### 2-(2-Chlorophenyl)-N-(4-{[5-methyl-2-(pyridin-3-yl)-1,3-thiazol-4-yl]oxy}-3-sulfamoylphenyl)acetamide

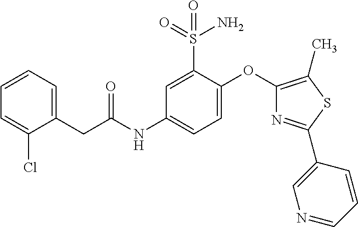 [SMILES: Cc1sc(-c2cccnc2)nc1Oc1ccc(NC(=O)Cc2ccccc2Cl)cc1S(N)(=O)=O]

According to general procedures GP1.3, GP2.3, GP3.2 and GP4, 2-chloro-N-(2,4-dimethoxybenzyl)-5-nitrobenzenesulfonamide (500 mg, 1.3 mmol), 5-methyl-2-(pyridin-3-yl)-1,3-thiazol-4-ol (373 mg, 1.9 mmol) and (2-chlorophenyl)acetic acid (52 mg, 0.3 mmol) were converted without purification of intermediates to 2-(2-chlorophenyl)-N-(4-{[5-methyl-2-(pyridin-3-yl)-1,3-thiazol-4-yl]oxy}-3-sulfamoylphenyl)acetamide. The pure compound was obtained after HPLC (Chromatorex C-18 10 μm, 125×30 mm, acetonitrile/water+0.1% formic acid) (8 mg, 0.015 mmol, 5% yield over 4 steps, 95% purity).

LC-MS (Method A): Rt=1.06 min

MS (ESIneg): m/z=513 (M−H)⁺

¹H-NMR (DMSO-d₆) δ [ppm]: 2.33 (s, 3H), 3.84 (s, 2H), 6.86-6.96 (m, 1H), 7.25-7.39 (m, 2H), 7.40-7.51 (m, 4H), 7.51-7.57 (m, 1H), 7.68-7.76 (m, 1H), 8.17-8.26 (m, 2H), 8.62-8.71 (m, 1H), 9.02-9.10 (m, 1H), 10.42-10.50 (m, 1H).

### Example 240

### N-[4-(3-Chlorophenoxy)-2-methyl-5-sulfamoylphenyl]-2-(2-chlorophenyl)acetamide

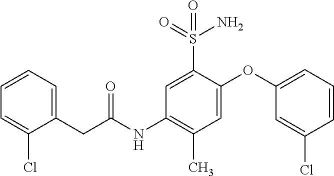 [SMILES: Cc1cc(Oc2cccc(Cl)c2)c(S(N)(=O)=O)cc1NC(=O)Cc1ccccc1Cl]

According to general procedures GP1.1, GP2.1, GP3.2 and GP4, N-(2,4-dimethoxybenzyl)-2-fluoro-4-methyl-5-nitrobenzenesulfonamide (100 mg, 0.260 mmol), 3-chlorophenol (38 mg, 0.286 mmol) and (2-chlorophenyl)acetic acid (97 mg, 0.6 mmol) were converted without purification of intermediates to N-[4-(3-chlorophenoxy)-2-methyl-5-sulfamoylphenyl]-2-(2-chlorophenyl)acetamide. The pure compound was obtained after preparative HPLC (Waters XBrigde C18 5μ 100×30 mm, acetonitrile/water+0.1% trifluoroacetic acid) (26 mg, 0.057 mmol, 8% yield over 4 steps, 96% purity).

LC-MS (Method I): Rt=2.66 min

MS (ESIpos): m/z=465 (M+H)⁺

¹H-NMR (DMSO-d₆) δ [ppm]: 2.22 (s, 3H), 3.89 (s, 2H), 6.98 (s, 2H), 7.11 (s, 1H), 7.17-7.24 (m, 1H), 7.29-7.50 (m, 7H), 7.92 (s, 1H), 9.70 (s, 1H).

### Example 241

### 2-(2-Chlorophenyl)-N-{4-[(1-oxidotetrahydrothiophen-3-yl)oxy]-3-sulfamoylphenyl}acetamide

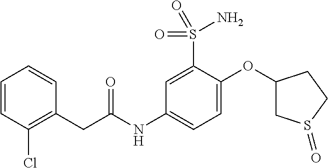 [SMILES: NS(=O)(=O)c1cc(NC(=O)Cc2ccccc2Cl)ccc1OC1CCS(=O)C1]

Iron(III)chloride (20 mg, 0.123 mmol) was dissolved in acetonitrile (0.5 mL) and added to 2-(2-chlorophenyl)-N-{3-[(2,4-dimethoxybenzyl)sulfamoyl]-4-[(3S)-tetrahydrothiophen-3-yloxy]phenyl}acetamide (560 mg, 1.0 mmol) in acetonitrile (25 mL). After 15 min periodic acid (243 mg, 1.1 mmol) was added and stirring was continued at room temperature for 30 min until TLC showed disappearance of starting material. Saturated aqueous bicarbonate solution and ethyl acetate were added and the phases were separated. The organic phase was dried and the solvent was removed under reduced pressure. The crude was purified by column chromatography on a Biotage Isolera system (silica gel, 1% gradient of ethanol in dichloromethane) to yield 2-(2-chlorophenyl)-N-(3-[(2,4-dimethoxybenzyl)sulfamoyl]-4-{[(1R,3S)-1-oxidotetrahydrothiophen-3-yl]oxy}phenyl)acetamide (100 mg, 0.230 mmol, 23% yield, 95% purity).

LC-MS (Method A): Rt=0.86 min

MS (ESIneg): m/z=441 (M−H)⁺

¹H-NMR (DMSO-d₆) δ [ppm]: 2.23-2.35 (m, 1H), 2.52-2.60 (m, 1H), 2.86-2.96 (m, 1H), 3.41-3.51 (m, 1H), 3.80 (s, 2H), 5.60-5.67 (m, 1H), 7.22 (s, 2H), 7.28-7.35 (m, 3H), 7.38-7.49 (m, 2H), 7.77-7.84 (m, 1H), 7.98-8.02 (m, 1H), 10.30 (s, 1H).

### Example 242

### N-[4-(4-Chlorophenoxy)-3-sulfamoylphenyl]-2-[2,6-dichloro-4-(trifluoromethyl)phenyl]acetamide

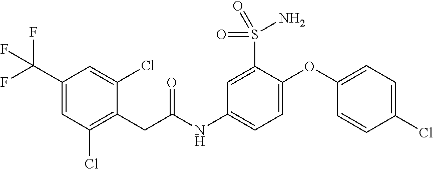 [SMILES: NS(=O)(=O)c1cc(NC(=O)Cc2c(Cl)cc(C(F)(F)F)cc2Cl)ccc1Oc1ccc(Cl)cc1]

According to GP3.1 and GP4 5-amino-2-(4-chlorophenoxy)-N-(2,4-dimethoxybenzyl)benzenesulfonamide (150 mg, 0.334 mmol) was reacted with [2,6-dichloro-4-(trifluoromethyl)phenyl]acetic acid (137 mg, 0.501 mmol) to N-[4-(4-chlorophenoxy)-3-sulfamoylphenyl]-2-[2,6-dichloro-4-(trifluoromethyl)phenyl]acetamide. The pure compound was obtained after preparative HPLC (Chromatorex C-18 10 μm, 125×30 mm, acetonitrile/water+0.1% formic acid) (37 mg, 0.064 mmol, 19% yield over 2 steps, 95% purity).

LC-MS (Method B): Rt=1.35 min

MS (ESIpos): m/z=553 (M+H)⁺

¹H-NMR (DMSO-d₆) δ [ppm]: 4.15 (s, 2H), 6.97-7.09 (m, 3H), 7.36 (s, 2H), 7.40-7.48 (m, 2H), 7.73 (dd, 1H), 7.94-7.99 (m, 2H), 8.19 (d, 1H), 10.65 (s, 1H).

### Example 243

### N-[4-(4-Chlorophenoxy)-3-sulfamoylphenyl]-2-(2,5-dichloro-4-cyanophenyl)acetamide

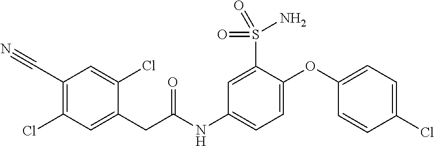 [SMILES: N#Cc1cc(Cl)c(CC(=O)Nc2ccc(Oc3ccc(Cl)cc3)c(S(N)(=O)=O)c2)cc1Cl]

According to GP3.1 and GP4 5-amino-2-(4-chlorophenoxy)-N-(2,4-dimethoxybenzyl)benzenesulfonamide (150 mg, 0.334 mmol) was reacted with (2,5-dichloro-4-cyanophenyl)acetic acid (115 mg, 0.501 mmol) to N-[4-(4-chlorophenoxy)-3-sulfamoylphenyl]-2-(2,5-dichloro-4-cyanophenyl)acetamide. The pure compound was obtained after preparative HPLC (Waters XBrigde C18 5μ 100×30 mm, acetonitrile/water+0.1% trifluoroacetic acid) (33 mg, 0.065 mmol, 20% yield over 2 steps, 95% purity).

LC-MS (Method A): Rt=1.22 min

MS (ESIpos): m/z=509 (M+H)⁺

¹H-NMR (DMSO-d₆) δ [ppm]: 3.97 (s, 2H), 6.97-7.09 (m, 3H), 7.33-7.48 (m, 4H), 7.69-7.78 (m, 1H), 7.90 (s, 1H), 8.12-8.18 (m, 1H), 8.24 (s, 1H), 10.57 (s, 1H).

### Example 244

### N-[4-(3-Chlorophenoxy)-3-sulfamoylphenyl]-2-phenylacetamide

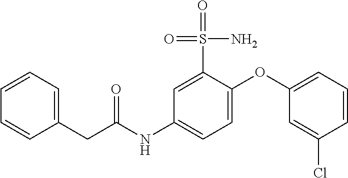 [SMILES: NS(=O)(=O)c1cc(NC(=O)Cc2ccccc2)ccc1Oc1cccc(Cl)c1]

N-{4-(3-Chlorophenoxy)-3-[(2,4-dimethoxybenzyl)sulfamoyl]phenyl}-2-phenylacetamide (150 mg, 0.264 mmol) was converted according to GP4 to N-[4-(3-chlorophenoxy)-3-sulfamoylphenyl]-2-phenylacetamide. The pure compound was obtained after preparative HPLC (Waters XBrigde C18 5μ 100×30 mm, acetonitrile/water+0.2% aqueous ammonia (32%)) (28 mg, 0.066 mmol, 25% yield, 95% purity).

LC-MS (Method B): Rt=min 1.12

MS (ESIpos): m/z=417 (M+H)⁺

¹H-NMR (400 MHz, DMSO-d₆) δ [ppm]: 3.65 (s, 2H), 6.92-7.00 (m, 1H), 7.04-7.11 (m, 2H), 7.15-7.22 (m, 1H), 7.22-7.30 (m, 1H), 7.30-7.45 (m, 7H), 7.77-7.85 (m, 1H), 8.16-8.23 (m, 1H), 10.46 (s, 1H).

### Example 245

### N-[4-(Cyclopropylmethoxy)-3-sulfamoylphenyl]-2-phenylacetamide

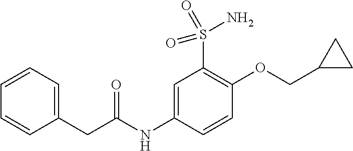 [SMILES: NS(=O)(=O)c1cc(NC(=O)Cc2ccccc2)ccc1OCC1CC1]

According to general procedure GP5, N-(4-hydroxy-3-sulfamoylphenyl)-2-phenylacetamide (153 mg, 0.5 mmol) and (iodomethyl)cyclopropane (109 mg, 0.6 mmol) were converted N-[4-(cyclopropylmethoxy)-3-sulfamoylphenyl]-2-phenylacetamide and was purified by preparative HPLC (Chromatorex C-18 10 μm, 125×30 mm, acetonitrile/water+0.1% formic acid) (30 mg, 0.0832 mmol, 17% yield, 95% purity).

LC-MS (Method A): Rt=1.03 min

MS (ESIpos): m/z=361 (M+H)⁺

¹H-NMR (400 MHz, METHANOL-d₄) δ [ppm]: 0.36-0.46 (m, 2H), 0.60-0.68 (m, 2H), 1.32-1.41 (m, 1H), 3.66 (s, 2H), 4.03 (d, 2H), 7.13 (d, 1H), 7.22-7.26 (m, 1H), 7.29-7.37 (m, 4H), 7.77 (dd, 1H), 8.02 (d, 1H).

### Example 246

### N-[4-(3,5-Dimethylphenoxy)-3-sulfamoylphenyl]-2-phenylacetamide

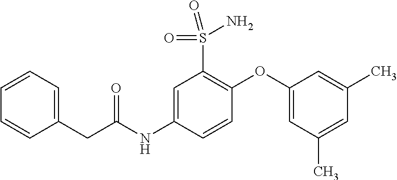 [SMILES: Cc1cc(C)cc(Oc2ccc(NC(=O)Cc3ccccc3)cc2S(N)(=O)=O)c1]

LC-MS (Method J): Rt=1.16 min

MS (ESIpos): m/z=411 (M+H)⁺

### Example 247

### N-[4-(2,4-Difluorophenoxy)-3-sulfamoylphenyl]-2-phenylacetamide

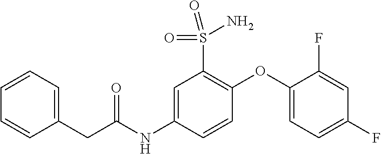 [SMILES: NS(=O)(=O)c1cc(NC(=O)Cc2ccccc2)ccc1Oc1ccc(F)cc1F]

LC-MS (Method J): Rt=1.03 min

MS (ESIpos): m/z=419 (M+H)⁺

### Example 248

### N-[4-(4-Fluorophenoxy)-3-sulfamoylphenyl]-2-phenylacetamide

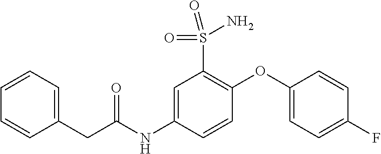 [SMILES: NS(=O)(=O)c1cc(NC(=O)Cc2ccccc2)ccc1Oc1ccc(F)cc1]

LC-MS (Method J): Rt=1.03 min

MS (ESIpos): m/z=401 (M+H)⁺

### Example 249

### N-[4-(3-Fluorophenoxy)-3-sulfamoylphenyl]-2-phenylacetamide

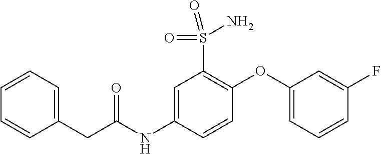 [SMILES: NS(=O)(=O)c1cc(NC(=O)Cc2ccccc2)ccc1Oc1cccc(F)c1]

LC-MS (Method J): Rt=1.04 min

MS (ESIpos): m/z=401 (M+H)⁺

### Example 250

### N-[4-(3-Methoxyphenoxy)-3-sulfamoylphenyl]-2-phenylacetamide

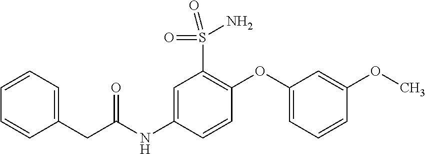 [SMILES: COc1cccc(Oc2ccc(NC(=O)Cc3ccccc3)cc2S(N)(=O)=O)c1]

LC-MS (Method J): Rt=1.03 min

MS (ESIpos): m/z=413 (M+H)⁺

### Example 251

### N-[4-(2-Fluoro-5-methylphenoxy)-3-sulfamoylphenyl]-2-phenylacetamide

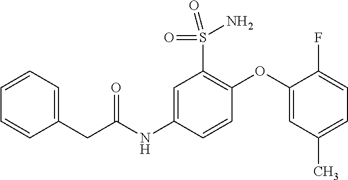 [SMILES: Cc1ccc(F)c(Oc2ccc(NC(=O)Cc3ccccc3)cc2S(N)(=O)=O)c1]

LC-MS (Method J): Rt=1.09 min

MS (ESIpos): m/z=415 (M+H)⁺

### Example 252

### 2-Phenyl-N-{3-sulfamoyl-4-[4-(trifluoromethoxy)phenoxy]phenyl}acetamide

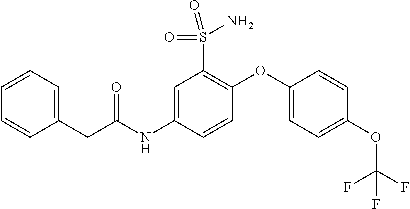 [SMILES: NS(=O)(=O)c1cc(NC(=O)Cc2ccccc2)ccc1Oc1ccc(OC(F)(F)F)cc1]

LC-MS (Method J): Rt=1.19 min

MS (ESIpos): m/z=466 (M−H)⁺

### Example 253

### 2-Phenyl-N-{3-sulfamoyl-4-[3-(trifluoromethyl)phenoxy]phenyl}acetamide

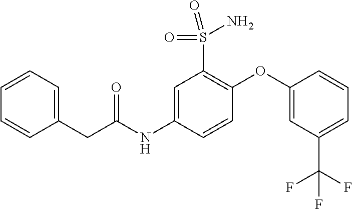 [SMILES: NS(=O)(=O)c1cc(NC(=O)Cc2ccccc2)ccc1Oc1cccc(C(F)(F)F)c1]

LC-MS (Method J): Rt=1.16 min

MS (ESIpos): m/z=451 (M+H)⁺

### Example 254

### N-[4-(3,5-Dimethoxyphenoxy)-3-sulfamoylphenyl]-2-phenylacetamide

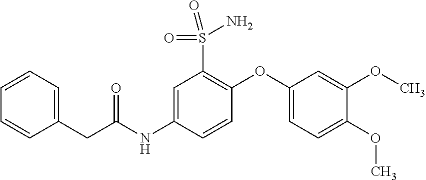 [SMILES: COc1ccc(Oc2ccc(NC(=O)Cc3ccccc3)cc2S(N)(=O)=O)cc1OC]

LC-MS (Method J): Rt=1.06 min

MS (ESIpos): m/z=443 (M+H)⁺

### Example 255

### N-[4-(3-Cyanophenoxy)-3-sulfamoylphenyl]-2-phenylacetamide

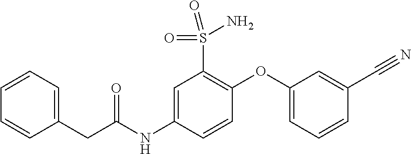 [SMILES: N#Cc1cccc(Oc2ccc(NC(=O)Cc3ccccc3)cc2S(N)(=O)=O)c1]

LC-MS (Method J): Rt=0.97 min

MS (ESIpos): m/z=408 (M+H)⁺

### Example 256

### N-[4-(3-Chlorophenoxy)-3-sulfamoylphenyl]-2-(4-hydroxyphenyl)acetamide

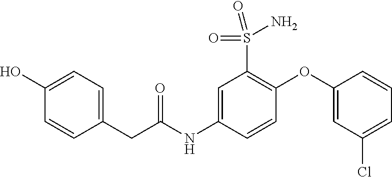 [SMILES: NS(=O)(=O)c1cc(NC(=O)Cc2ccc(O)cc2)ccc1Oc1cccc(Cl)c1]

A 1 00-mL Erlenmeyer flask containing sterile growth medium (20 mL) was inoculated with a DMSO cryo culture (0.2 mL) of Cunninghamella bainieri (ATCC 9244). The growth medium consisting of D-(+)-glucose monohydrate (30 g/L), corn steep liquor (10 g/L), sodium nitrate (2 g/L), monopotassium phosphate (1 g/L), dipotassium phosphate (2 g/L), potassium chloride (0.5 g/L), magnesium sulfate heptahydrate (0.5 g/L), and iron(II) sulfate heptahydrate (20 mg/L), was adjusted to pH 6 with sodium hydroxide solution (16% in water) and sterilized at 121° C. for 20 minutes. After inoculation, the growth flask was shaken on a rotation shaker (165 rpm) at 27° C. for 65 hours. A 500-mL Erlenmeyer flask containing the same sterile growth medium (100 mL, prepared under the same conditions) was inoculated with the preculture (10 mL). Then, the flask was shaken on a rotation shaker (rpm 165) at 27° C. for 48 hours.

A 10-L fermenter was filled with the same growth medium (9.3 L) and adjusted to pH 6. Silicon oil (0.5 mL) and Synperonic (0.5 mL) were added, and it was sterilized at 121° C. for 40 minutes. The culture of the 500-mL Erlenmeyer flask was added to the fermenter under sterile conditions. The fermenter was operated under gauge pressure (0.7 bar), aerated with air (5 L/min) and stirred (350 rpm) at 27° C. After 10 hours, N-[4-(3-chlorophenoxy)-3-sulfamoylphenyl]-2-phenylacetamide (200 mg, 0.48 mmol) dissolved in DMF (25 mL) was added and the fermentation was continued for 115.5 hours.

The culture broth was extracted with methyl isobutyl ketone (15 L) for 18 hours. The organic phase was concentrated to dryness. The residue was treated with methanol (150 mL) and water (15 mL). This solution was extracted twice with n-hexane (100 mL). The methanol/water layer was concentrated to dryness. The residue was purified by column chromatography on a Biotage Isolera system (silica gel, n-hexane/ethyl acetate gradient), followed by preparative HPLC (Phenomenex Kinetex C18 5μ, 100×30 mm, acetonitrile/water+0.1% formic acid), affording N-[4-(3-chlorophenoxy)-3-sulfamoylphenyl]-2-(4-hydroxyphenyl)acetamide (2.5 mg, 0.00578 mmol, 1% yield, 97% purity).

LC-MS (Method A): Rt=1.11 min

MS (ESIpos): m/z=417 (M+H)⁺

¹H-NMR (600 MHz, DMSO-d₆) δ [ppm]: 3.50 (s, 2H), 6.71 (d, 2H), 6.94-6.99 (m, 1H), 7.05-7.09 (m, 2H), 7.12 (d, 2H), 7.16-7.20 (m, 1H), 7.34-7.41 (m, 3H), 7.79-7.83 (m, 1H), 8.18-8.20 (m, 1H), 9.27 (s, 1H), 10.37 (s, 1H).

### Example 257

### 2-(2-Chloro-6-methoxy-4-methylphenyl)-N-[4-(3-chlorophenoxy)-3-sulfamoylphenyl]acetamide

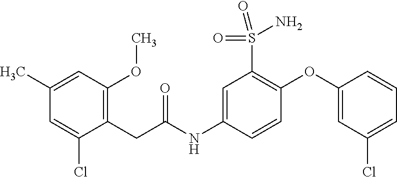 [SMILES: COc1cc(C)cc(Cl)c1CC(=O)Nc1ccc(Oc2cccc(Cl)c2)c(S(N)(=O)=O)c1]

According to general procedures GP3.2 and GP4, purified 5-amino-2-(3-chlorophenoxy)-N-(2,4-dimethoxybenzyl)benzenesulfonamide (70.0 mg, 0.156 mmol) and (2-chloro-6-methoxy-4-methylphenyl)acetic acid (36.8 mg, 0.172 mmol) were converted to 2-(2-chloro-6-methoxy-4-methylphenyl)-N-[4-(3-chlorophenoxy)-3-sulfamoylphenyl]acetamide and were purified twice by preparative HPLC (Waters XBrigde C18 5μ 100×30 mm, acetonitrile/water+0.1% formic acid) (1.4 mg, 0.00283 mmol, 2% yield, 95% purity).

LC-MS (Method A): Rt=1.28 min

MS (ESIpos): m/z=495 (M+H)⁺

¹H-NMR (400 MHz, DMSO-d₆) δ [ppm]: 2.31 (s, 3H), 3.76-3.81 (m, 5H), 6.82-6.85 (m, 1H), 6.87-6.91 (m, 1H), 6.93-7.00 (m, 1H), 7.06-7.10 (m, 2H), 7.16-7.22 (m, 1H), 7.39 (s, 3H), 7.74-7.80 (m, 1H), 8.15-8.24 (m, 1H), 10.40-10.45 (m, 1H).

### Example 258

### 2-(2-Chloro-6-fluorophenyl)-N-[4-(3-chlorophenoxy)-3-sulfamoylphenyl]propanamide

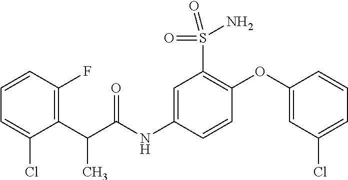 [SMILES: CC(C(=O)Nc1ccc(Oc2cccc(Cl)c2)c(S(N)(=O)=O)c1)c1c(F)cccc1Cl]

According to general procedures GP3.2 and GP4, purified 5-amino-2-(3-chlorophenoxy)-N-(2,4-dimethoxybenzyl)benzenesulfonamide (100 mg, 0.223 mmol) and 2-(2-chloro-6-fluorophenyl)propanoic acid (49.6 mg, 0.245 mmol) were converted to 2-(2-chloro-6-fluorophenyl)-N-[4-(3-chlorophenoxy)-3-sulfamoylphenyl]propanamide and were purified by preparative HPLC (Waters XBrigde C18 5μ 100×30 mm, acetonitrile/water+0.1% formic acid) (61 mg, 0.126 mmol, 55% yield, 95% purity).

LC-MS (Method A): Rt=1.30 min

MS (ESIpos): m/z=483 (M+H)⁺

¹H-NMR (400 MHz, DMSO-d₆) δ [ppm]: 1.43 (d, 3H), 4.24 (q, 1H), 6.90-7.03 (m, 1H), 7.04-7.11 (m, 2H), 7.16-7.28 (m, 2H), 7.32-7.46 (m, 5H), 7.84 (dd, 1H), 8.13 (d, 1H), 9.95 (s, 1H).

### Example 259

### 2-(2-Chloro-4,6-difluorophenyl)-N-[4-(3-chlorophenoxy)-3-sulfamoylphenyl]acetamide

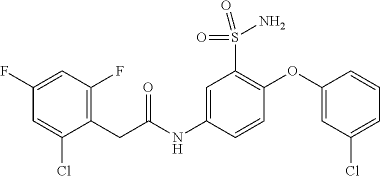 [SMILES: NS(=O)(=O)c1cc(NC(=O)Cc2c(F)cc(F)cc2Cl)ccc1Oc1cccc(Cl)c1]

According to general procedures GP3.2 and GP4, purified 5-amino-2-(3-chlorophenoxy)-N-(2,4-dimethoxybenzyl)benzenesulfonamide (100 mg, 0.223 mmol) and (2-chloro-4,6-difluorophenyl)acetic acid (50.6 mg, 0.245 mmol) were converted to 2-(2-chloro-4,6-difluorophenyl)-N-[4-(3-chlorophenoxy)-3-sulfamoylphenyl]acetamide and were purified by preparative HPLC (Waters XBrigde C18 5μ 100×30 mm, acetonitrile/water+0.1% formic acid) followed by another preparative HPLC (Waters XBrigde C18 5μ 100×30 mm, acetonitrile/water+0.2% aqueous ammonia (32%)) (21 mg, 0.0431 mmol, 19% yield, 97% purity).

LC-MS (Method B): Rt=1.19 min

MS (ESIpos): m/z=487 (M+H)⁺

¹H-NMR (400 MHz, DMSO-d₆) δ [ppm]: 3.87 (s, 2H), 6.93-7.05 (m, 1H), 7.05-7.14 (m, 2H), 7.15-7.24 (m, 1H), 7.32-7.47 (m, 5H), 7.71-7.83 (m, 1H), 8.20 (d, 1H), 10.62 (s, 1H).

### Example 260

### N-[4-(3-Chlorophenoxy)-3-sulfamoylphenyl]-2-(2,6-dichlorophenyl)propanamide

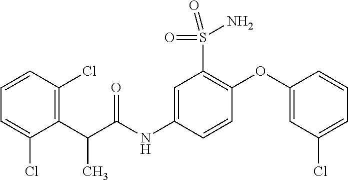 [SMILES: CC(C(=O)Nc1ccc(Oc2cccc(Cl)c2)c(S(N)(=O)=O)c1)c1c(Cl)cccc1Cl]

According to general procedures GP3.2 and GP4, purified 5-amino-2-(3-chlorophenoxy)-N-(2,4-dimethoxybenzyl)benzenesulfonamide (100 mg, 0.223 mmol) and 2-(2,6-dichlorophenyl)propanoic acid (53.7 mg, 0.245 mmol) were converted to N-[4-(3-chlorophenoxy)-3-sulfamoylphenyl]-2-(2,6-dichlorophenyl)propanamide and were purified by preparative HPLC (Waters XBrigde C18 5μ 100×30 mm, acetonitrile/water+0.1% formic acid) (32 mg, 0.0640 mmol, 29% yield, 95% purity).

LC-MS (Method A): Rt=1.34 min

MS (ESIpos): m/z=499 (M+H)⁺

¹H-NMR (400 MHz, DMSO-d₆) δ [ppm]: 1.43 (d, 3H), 4.36-4.50 (m, 1H), 6.93-7.02 (m, 1H), 7.02-7.13 (m, 2H), 7.17-7.23 (m, 1H), 7.29-7.46 (m, 4H), 7.46-7.55 (m, 2H), 7.87 (dd, 1H), 8.11 (d, 1H), 9.69 (s, 1H).

### Example 261

### 2-(2-Chlorophenyl)-N-{4-[(²H₅)phenyloxy]-3-sulfamoylphenyl}acetamide

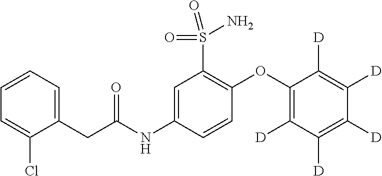 [SMILES: [2H]c1c([2H])c([2H])c(Oc2ccc(NC(=O)Cc3ccccc3Cl)cc2S(N)(=O)=O)c([2H])c1[2H]]

According to general procedures GP1.1, GP2.2, GP3.2 and GP4, 2-chloro-N-(2,4-dimethoxybenzyl)-5-nitrobenzenesulfonamide (500 mg, 1.29 mmol), (²H₅)phenol (128 mg, 1.29 mmol) and (2-chlorophenyl)acetic acid (220 mg, 1.29 mmol) were converted without purification of intermediates to 2-(2-chlorophenyl)-N-{4-[(²H₅)phenyloxy]-3-sulfamoylphenyl}acetamide and were purified at the end by preparative HPLC (Waters XBrigde C18 5μ 100×30 mm, acetonitrile/water+0.1% formic acid) (9 mg, 0.0213 mmol, 2% yield over 4 steps, 99% purity).

LC-MS (Method A): Rt=1.12 min

MS (ESIpos): m/z=422 (M+H)⁺

¹H-NMR (400 MHz, DMSO-d₆) δ [ppm]: 3.84 (s, 2H), 6.94 (d, 1H), 7.26-7.37 (m, 4H), 7.40-7.48 (m, 2H), 7.74 (dd, 1H), 8.19 (d, 1H), 10.48 (s, 1H).

### Example 262

### 2-(2-Chlorophenyl)-N-(4-{[4-chloro(²H₄)phenyl]oxy}-3-sulfamoylphenyl)acetamide

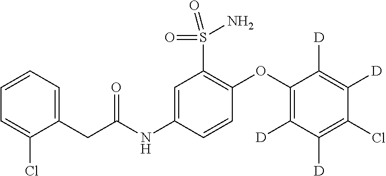 [SMILES: [2H]c1c([2H])c(Oc2ccc(NC(=O)Cc3ccccc3Cl)cc2S(N)(=O)=O)c([2H])c([2H])c1Cl]

According to general procedures GP1.1, GP2.2, GP3.2 and GP4, 2-chloro-N-(2,4-dimethoxybenzyl)-5-nitrobenzenesulfonamide (500 mg, 1.29 mmol), 4-chloro(²H₄)phenol (171 mg, 1.29 mmol) and (2-chlorophenyl)acetic acid (220 mg, 1.29 mmol) were converted without purification of intermediates to 2-(2-chlorophenyl)-N-(4-{[4-chloro(²H₄)phenyl]oxy}-3-sulfamoylphenyl)acetamide and were purified at the end by preparative HPLC (Waters XBrigde C18 5μ 100×30 mm, acetonitrile/water+0.1% formic acid) (14 mg, 0.0307 mmol, 2% yield over 4 steps, 99% purity).

LC-MS (Method A): Rt=1.21 min

MS (ESIpos): m/z=455 (M+H)⁺

¹H-NMR (400 MHz, DMSO-d₆) δ [ppm]: 3.84 (s, 2H), 7.02 (d, 1H), 7.26-7.36 (m, 2H), 7.38 (s, 2H), 7.41-7.47 (m, 2H), 7.77 (dd, 1H), 8.20 (d, 1H), 10.51 (s, 1H).

### Example 263

### 2-(2-Chlorophenyl)-N-(4-{[2-chloro(²H₄)phenyl]oxy}-3-sulfamoylphenyl)acetamide

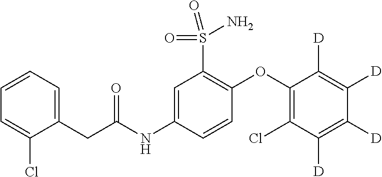 [SMILES: [2H]c1c([2H])c([2H])c(Oc2ccc(NC(=O)Cc3ccccc3Cl)cc2S(N)(=O)=O)c(Cl)c1[2H]]

According to general procedures GP1.1, GP2.2, GP3.2 and GP4, 2-chloro-N-(2,4-dimethoxybenzyl)-5-nitrobenzenesulfonamide (500 mg, 1.29 mmol), 2-chloro(²H₄)phenol (171 mg, 1.29 mmol) and (2-chlorophenyl)acetic acid (220 mg, 1.29 mmol) were converted without purification of intermediates to 2-(2-chlorophenyl)-N-(4-{[2-chloro(²H₄)phenyl]oxy}-3-sulfamoylphenyl)acetamide and were purified at the end by preparative HPLC (Waters XBrigde C18 5μ 100×30 mm, acetonitrile/water+0.1% formic acid) (9 mg, 0.0198 mmol, 2% yield over 4 steps, 99% purity).

LC-MS (Method A): Rt=1.16 min

MS (ESIpos): m/z=455 (M+H)⁺

¹H-NMR (400 MHz, DMSO-d₆) δ [ppm]: 3.83 (s, 2H), 6.73 (d, 1H), 7.28-7.35 (m, 2H), 7.38 (s, 2H), 7.40-7.48 (m, 2H), 7.71 (dd, 1H), 8.20 (d, 1H), 10.47 (s, 1H).

### Example 264

### 2-(2-Chlorophenyl)-N-{4-[4-(2-hydroxypropan-2-yl)phenoxy]-3-sulfamoylphenyl}acetamide

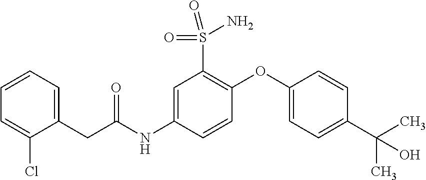 [SMILES: CC(C)(O)c1ccc(Oc2ccc(NC(=O)Cc3ccccc3Cl)cc2S(N)(=O)=O)cc1]

Methyl 4-(4-{[(2-chlorophenyl)acetyl]amino}-2-sulfamoylphenoxy)benzoate (60 mg, 0.13 mmol) was dissolved in tetrahydrofuran (2.5 mL) and a solution of methyl magnesium bromide (0.9 mL, 1.4M) was added under a nitrogen atmosphere slowly at 0° C. It was stirred at 0° C. for 2 h, followed by stirring at room temperature overnight. Then, again a solution of methyl magnesium bromide (0.3 mL, 1.4M) was added at 0° C. It was continued stirring at 0° C. for 2 hours and quenched at the same temperature with saturated aqueous ammonium chloride solution and extracted twice with ethyl acetate. The combined ethyl acetate phases were washed with brine, dried over sodium sulfate and concentrated in vacuo. Purification by preparative HPLC (Chromatorex C-18 10 μm, 125×30 mm, acetonitrile/water+0.1% aqueous ammonia (32%)) led to 2-(2-chlorophenyl)-N-{4-[4-(2-hydroxypropan-2-yl)phenoxy]-3-sulfamoylphenyl}acetamide (23 mg, 0.0484, 37% yield, 99% purity).

LC-MS (Method B): Rt=1.01 min

MS (ESIneg): m/z=473 (M−H)⁺

¹H-NMR (400 MHz, DMSO-d₆) δ [ppm]: 1.42 (s, 6H), 3.83 (s, 2H), 5.02 (s, 1H), 6.92 (d, 1H), 6.95-6.99 (m, 2H), 7.31 (m, 4H), 7.40-7.51 (m, 4H), 7.73 (dd, 1H), 8.18 (d, 1H), 10.48 (s, 1H).

### Example 265

### 2-(2-Chlorophenyl)-N-{4-[(2,2-dimethyltetrahydro-2H-pyran-4-yl)methoxy]-3-sulfamoylphenyl}acetamide

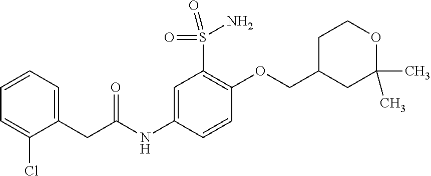 [SMILES: CC1(C)CC(COc2ccc(NC(=O)Cc3ccccc3Cl)cc2S(N)(=O)=O)CCO1]

According to general procedures GP2.1 (but with methanol as solvent), GP3.2 and GP4, N-(2,4-dimethoxybenzyl)-2-[(2,2-dimethyltetrahydro-2H-pyran-4-yl)methoxy]-5-nitrobenzenesulfonamide (154 mg, 0.31 mmol) and (2-chlorophenyl)acetic acid (39.6 mg, 0.23 mmol) were converted without purification of intermediates to 2-(2-chlorophenyl)-N-{4-[(2,2-dimethyltetrahydro-2H-pyran-4-yl)methoxy]-3-sulfamoylphenyl}acetamide and were purified at the end by preparative HPLC (Chromatorex C-18 10 μm, 125×30 mm, acetonitrile/water+0.1% formic acid), followed by another preparative HPLC (Waters XBrigde C18 5μ 100×30 mm, acetonitrile/water+0.2% aqueous ammonia (32%)) (6.9 mg, 0.0148 mmol, 5% yield over 3 steps, 98% purity).

LC-MS (Method B): Rt=1.05 min

MS (ESIpos): m/z=467 (M+H)⁺

¹H-NMR (400 MHz, DMSO-d₆) δ [ppm]: 1.06-1.20 (m, 8H), 1.58-1.66 (m, 1H), 1.72-1.80 (m, 1H), 1 signal below solvent, 3.56-3.69 (m, 2H), 3.76-4.00 (m, 4H), 6.90 (s, 2H), 7.15 (d, 1H), 7.27-7.35 (m, 2H), 7.38-7.50 (m, 2H), 7.76 (dd, 1H), 8.02 (d, 1H), 10.31 (s, 1H).

### Example 266

### 2-(2-Chlorophenyl)-N-{4-[(1R,5S,6r)-3-oxabicyclo[3.1.0]hex-6-ylmethoxy]-3-sulfamoylphenyl}acetamide

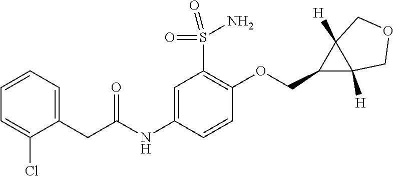 [SMILES: NS(=O)(=O)c1cc(NC(=O)Cc2ccccc2Cl)ccc1OC[C@H]1[C@@H]2COC[C@@H]21]

According to general procedures GP1.1, GP2.1 (but with methanol as solvent), GP3.2 and GP4, 2-chloro-N-(2,4-dimethoxybenzyl)-5-nitrobenzenesulfonamide (500 mg, 1.29 mmol), (1R,5S,6r)-3-oxabicyclo[3.1.0]hex-6-ylmethanol (Achemblocks F-4895, 148 mg, 1.29 mmol) and (2-chlorophenyl)acetic acid (181 mg, 1.06 mmol) were converted without purification of intermediates to 2-(2-chlorophenyl)-N-[4-(3-oxabicyclo[3.1.0]hex-6-ylmethoxy)-3-sulfamoylphenyl]acetamide and were purified at the end by preparative HPLC (Waters XBrigde C18 5μ 100×30 mm, acetonitrile/water+0.2% aqueous ammonia (32%)) (4.7 mg, 0.0108 mmol, 1% yield over 4 steps, 97% purity).

LC-MS (Method B): Rt=1.10 min

MS (ESIneg): m/z=435 (M−H)⁺

¹H-NMR (400 MHz, DMSO-d₆) δ [ppm]: 1.18 (m, 1H), 1.76 (m, 2H), 3.56 (d, 2H), 3.74 (d, 2H), 3.81 (s, 2H), 4.07 (d, 2H), 6.90 (s, 2H), 7.19 (d, 1H), 7.26-7.37 (m, 2H), 7.38-7.49 (m, 2H), 7.75 (dd, 1H), 8.02 (d, 1H), 10.30 (s, 1H).

### Example 267

### 2-(2-Chlorophenyl)-N-{4-[(4-chlorotetrahydro-2H-pyran-4-yl)methoxy]-3-sulfamoylphenyl}acetamide

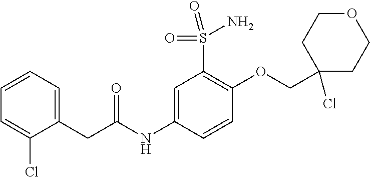 [SMILES: NS(=O)(=O)c1cc(NC(=O)Cc2ccccc2Cl)ccc1OCC1(Cl)CCOCC1]

According to general procedures GP1.1, GP2.1 (but with methanol as solvent), GP3.2 and GP4, 2-chloro-N-(2,4-dimethoxybenzyl)-5-nitrobenzenesulfonamide (500 mg, 1.29 mmol), (1-chlorocyclohexyl)methanol (195 mg, 1.29 mmol) and (2-chlorophenyl)acetic acid (155 mg, 0.91 mmol) were converted without purification of intermediates to 2-(2-chlorophenyl)-N-{4-[(4-chlorotetrahydro-2H-pyran-4-yl)methoxy]-3-sulfamoylphenyl}acetamide and were purified at the end twice by preparative HPLC (Waters XBrigde C18 5μ 100×30 mm, acetonitrile/water+0.1% formic acid) (3.6 mg, 0.00760 mmol, 1% yield over 4 steps, 75% purity).

LC-MS (Method A): Rt=1.00 min

MS (ESIneg): m/z=471 (M−H)⁺

### Example 268

### 2-(2-Chlorophenyl)-N-[4-(1,4-dioxan-2-ylmethoxy)-3-sulfamoylphenyl]acetamide

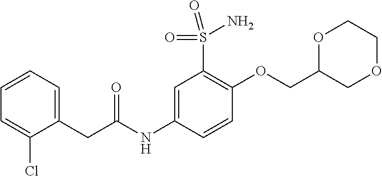 [SMILES: NS(=O)(=O)c1cc(NC(=O)Cc2ccccc2Cl)ccc1OCC1COCCO1]

According to general procedures GP1.1, GP2.1 (but with methanol as solvent), GP3.2 and GP4, 2-chloro-N-(2,4-dimethoxybenzyl)-5-nitrobenzenesulfonamide (500 mg, 1.29 mmol), 1,4-dioxan-2-ylmethanol (153 mg, 1.29 mmol) and (2-chlorophenyl)acetic acid (154 mg, 0.90 mmol) were converted without purification of intermediates to 2-(2-chlorophenyl)-N-[4-(1,4-dioxan-2-ylmethoxy)-3-sulfamoylphenyl]acetamide and were purified at the end twice by preparative HPLC (Waters XBrigde C18 5μ 100×30 mm, acetonitrile/water+0.1% formic acid) (7 mg, 0.0159 mmol, 1% yield over 4 steps, 98% purity).

LC-MS (Method B): Rt=0.89 min

MS (ESIneg): m/z=439 (M−H)+

¹H-NMR (400 MHz, DMSO-d₆) δ [ppm]: 3.37-3.52 (m, 2H), 3.61-3.70 (m, 2H), 3.71-3.85 (m, 3H), 3.85-3.92 (m, 1H), 3.91-4.03 (m, 1H), 4.06-4.15 (m, 2H), 6.92 (s, 2H), 7.19 (d, 1H), 7.25-7.36 (m, 2H), 7.38-7.50 (m, 2H), 7.77 (dd, 1H), 8.02 (d, 1H), 10.35 (s, 1H).

### Example 269

### 2-(2-Chlorophenyl)-N-{3-sulfamoyl-4-[(2,2,6,6-tetramethyltetrahydro-2H-pyran-4-yl)oxy]phenyl}acetamide

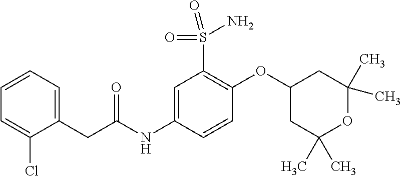 [SMILES: CC1(C)CC(Oc2ccc(NC(=O)Cc3ccccc3Cl)cc2S(N)(=O)=O)CC(C)(C)O1]

2,2,6,6-Tetramethyltetrahydro-2H-pyran-4-ol (251 mg, 1.59 mmol) was dissolved in dimethylformamide (10 mL) and treated with sodium hydride (296 mg, 7.40 mmol, purity 60%). After 10 min 2-chloro-5-nitrobenzenesulfonamide (250 mg, 1.06 mmol) was added and it was stirred overnight at room temperature. It was quenched at 0° C. with water and the solvent was removed in vacuo. Water and dichloromethane were added, it was extracted with dichloromethane, the organic phases were combined, washed with brine and dried over sodium sulfate prior to being concentrated in vacuo yielding crude 5-nitro-2-[(2,2,6,6-tetramethyltetrahydro-2H-pyran-4-yl)oxy]benzenesulfonamide (286 mg).

The crude material from the previous step was dissolved in methanol (3 mL), flushed with nitrogen and treated with Pd/C (30 mg, 0.28 mmol) under a hydrogen atmosphere (1 bar). After stirring for 6 hours at room temperature the catalyst was filtered off, it was washed with methanol followed by concentration in vacuo yielding crude 5-amino-2-[(2,2,6,6-tetramethyltetrahydro-2H-pyran-4-yl)oxy]benzenesulfonamide (126 mg).

The crude material from the previous step was dissolved in dimethylformamide (3 mL) and treated with (2-chlorophenyl)acetic acid (71.4 mg, 0.42 mmol), N,N-diisopropylethylamine (148 mg, 1.14 mmol) and HATU (159 mg, 0.42 mmol). The reaction mixture was stirred overnight at room temperature, then it was concentrated in vacuo. Ethyl acetate and water were added, the organic phase was dried and concentrated in vacuo. Purification by preparative HPLC (Chromatorex C-18 10 μm, 125×30 mm, acetonitrile/water+0.1% aqueous ammonia (32%)) led to 2-(2-chlorophenyl)-N-{3-sulfamoyl-4-[(2,2,6,6-tetramethyltetrahydro-2H-pyran-4-yl)oxy]phenyl}acetamide (25 mg, 0.0520 mmol, 3% yield over 3 steps, 98% purity).

LC-MS (Method B): Rt=1.17 min

MS (ESIneg): m/z=479 (M−H)⁺

¹H-NMR (400 MHz, DMSO-d₆) δ [ppm]: 1.16 (s, 6H), 1.29 (s, 6H), 1.52 (t, 2H), 2.01 (dd, 2H), 3.81 (s, 2H), 4.93-5.04 (m, 1H), 6.85 (s, 2H), 7.24-7.35 (m, 3H), 7.38-7.48 (m, 2H), 7.76 (dd, 1H), 8.03 (d, 1H), 10.32 (s, 1H).

### Example 270

### N-[4-(3-Chlorophenoxy)-3-methyl-5-sulfamoylphenyl]-2-(2-chlorophenyl)acetamide

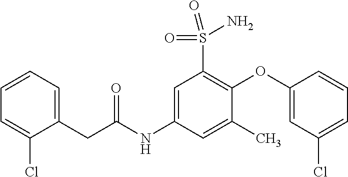 [SMILES: Cc1cc(NC(=O)Cc2ccccc2Cl)cc(S(N)(=O)=O)c1Oc1cccc(Cl)c1]

According to general procedures GP3.2 and GP4, crude 5-amino-2-(3-chlorophenoxy)-N-(2,4-dimethoxybenzyl)-3-methylbenzenesulfonamide (125 mg) and (2-chlorophenyl)acetic acid (50.6 mg, 0.297 mmol) were converted to N-[4-(3-chlorophenoxy)-3-methyl-5-sulfamoylphenyl]-2-(2-chlorophenyl)acetamide and were purified twice by preparative HPLC (Waters XBrigde C18 5μ 100×30 mm, acetonitrile/water+0.1% formic acid) (5 mg, 0.0107 mmol, 97% purity).

LC-MS (Method A): Rt=1.23 min

MS (ESIpos): m/z=465 (M+H)⁺

¹H-NMR (400 MHz, DMSO-d₆) δ [ppm]: 1.15 (s, 3H), 3.02 (s, 2H), 5.83-5.91 (m, 1H), 6.03-6.08 (m, 1H), 6.20-6.27 (m, 1H), 6.40-6.56 (m, 5H), 6.57-6.66 (m, 2H), 6.86-7.07 (m, 1H), 7.24 (d, 1H), 9.71 (s, 1H).

### Example 271

### N-[4-(3-Chlorophenoxy)-3-methyl-5-sulfamoylphenyl]-2-phenylacetamide

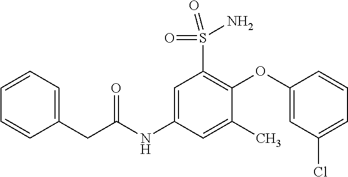 [SMILES: Cc1cc(NC(=O)Cc2ccccc2)cc(S(N)(=O)=O)c1Oc1cccc(Cl)c1]

According to general procedures GP3.2 and GP4, crude 5-amino-2-(3-chlorophenoxy)-N-(2,4-dimethoxybenzyl)-3-methylbenzenesulfonamide (200 mg) and phenylacetic acid (64.7 mg, 0.475 mmol) were converted to N-[4-(3-chlorophenoxy)-3-methyl-5-sulfamoylphenyl]-2-phenylacetamide and were purified by preparative HPLC (Waters XBrigde C18 5μ 100×30 mm, acetonitrile/water+0.1% formic acid) (8 mg, 0.0186 mmol, 97% purity).

LC-MS (Method A): Rt=1.21 min

MS (ESIpos): m/z=429 (M+H)⁺

### Example 272

### Methyl 2-(4-{[(2-chlorophenyl)acetyl]amino}-2-sulfamoylphenoxy)benzoate

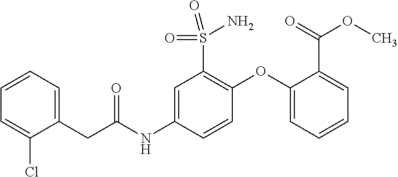 [SMILES: COC(=O)c1ccccc1Oc1ccc(NC(=O)Cc2ccccc2Cl)cc1S(N)(=O)=O]

According to GP4 methyl 2-(4-{[(2-chlorophenyl)acetyl]amino}-2-[(2,4-dimethoxybenzyl)sulfamoyl]phenoxy)benzoate (350 mg, 0.56 mmol) was converted to methyl 2-(4-{[(2-chlorophenyl)acetyl]amino}-2-sulfamoylphenoxy)benzoate and was purified by preparative HPLC (Chromatorex C-18 10 μm, 125×30 mm, acetonitrile/water+0.1% formic acid) (54 mg, 0.114 mmol, 20% yield, 97% purity).

LC-MS (Method A): Rt=1.14 min

MS (ESIpos): m/z=475 (M+H)⁺

¹H-NMR (400 MHz, DMSO-d₆) δ [ppm]: 3.79 (s, 3H), 3.85 (s, 2H), 6.97 (d, 1H), 7.05 (d, 1H), 7.21-7.38 (m, 5H), 7.38-7.50 (m, 2H), 7.53-7.63 (m, 1H), 7.74-7.82 (m, 1H), 7.87-7.92 (m, 1H), 8.22 (d, 1H), 10.52 (s, 1H).

### Example 273

### Methyl 4-(4-{[(2-chlorophenyl)acetyl]amino}-2-sulfamoylphenoxy)benzoate

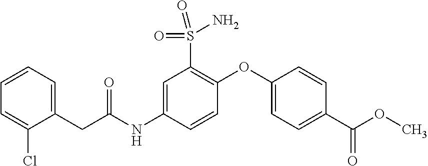 [SMILES: COC(=O)c1ccc(Oc2ccc(NC(=O)Cc3ccccc3Cl)cc2S(N)(=O)=O)cc1]

According to GP4 methyl 4-(4-{[(2-chlorophenyl)acetyl]amino}-2-[(2,4-dimethoxybenzyl)sulfamoyl]phenoxy)benzoate (441 mg, 0.705 mmol) was converted to methyl 4-(4-{[(2-chlorophenyl)acetyl]amino}-2-sulfamoylphenoxy)benzoate and was purified by preparative HPLC (Chromatorex C-18 10 μm, 125×30 mm, acetonitrile/water+0.1% formic acid) (53 mg, 0.112 mmol, 16% yield, 97% purity).

LC-MS (Method A): Rt=1.11 min

MS (ESIpos): m/z=475 (M+H)⁺

¹H-NMR (400 MHz, DMSO-d₆) δ [ppm]: 3.84 (s, 3H), 3.86 (s, 2H), 7.04-7.16 (m, 3H), 7.26-7.38 (m, 2H), 7.39-7.50 (m, 4H), 7.83 (dd, 1H), 7.92-8.01 (m, 2H), 8.23 (d, 1H), 10.55 (s, 1H).

### Example 274

### 2-(2-Chlorophenyl)-N-{4-[3-(2-hydroxypropan-2-yl)phenoxy]-3-sulfamoylphenyl}acetamide

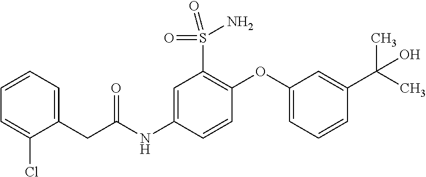 [SMILES: CC(C)(O)c1cccc(Oc2ccc(NC(=O)Cc3ccccc3Cl)cc2S(N)(=O)=O)c1]

Methyl 3-(4-{[(2-chlorophenyl)acetyl]amino}-2-sulfamoylphenoxy)benzoate (50 mg, 0.11 mmol) was dissolved in tetrahydrofuran (2.5 mL) and a solution of methyl magnesium bromide (0.9 mL, 1.4M) was added under a nitrogen atmosphere slowly at 0° C. It was stirred at 0° C. for 2 h, followed by stirring at room temperature overnight. Then, again a solution of methyl magnesium bromide (0.75 mL, 1.4M) was added at 0° C. It was continued stirring at 0° C. for 2 hours and quenched at the same temperature with saturated aqueous ammonium chloride solution and extracted twice with ethyl acetate. The combined ethyl acetate phases were washed with brine, dried over sodium sulfate and concentrated in vacuo. Purification by preparative HPLC (Chromatorex C-18 10 μm, 125×30 mm, acetonitrile/water+0.1% aqueous ammonia (32%)) led to 2-(2-chlorophenyl)-N-{4-[3-(2-hydroxypropan-2-yl)phenoxy]-3-sulfamoylphenyl}acetamide (24 mg, 0.0505, 46% yield, 98% purity).

LC-MS (Method B): Rt=1.04 min

MS (ESIneg): m/z=473 (M−H)⁺

¹H-NMR (400 MHz, DMSO-d₆) δ [ppm]: 1.41 (s, 6H), 3.84 (s, 2H), 5.08 (s, 1H), 6.79-6.92 (m, 2H), 7.18-7.37 (m, 7H), 7.40-7.49 (m, 2H), 7.69-7.78 (m, 1H), 8.14-8.21 (m, 1H), 10.47 (s, 1H).

### Example 275

### 2-(2-Chlorophenyl)-N-{4-[2-(2-hydroxypropan-2-yl)phenoxy]-3-sulfamoylphenyl}acetamide

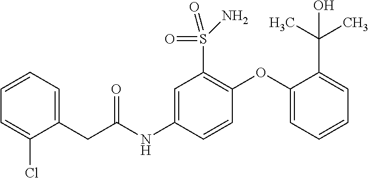 [SMILES: CC(C)(O)c1ccccc1Oc1ccc(NC(=O)Cc2ccccc2Cl)cc1S(N)(=O)=O]

Methyl 2-(4-{[(2-chlorophenyl)acetyl]amino}-2-sulfamoylphenoxy)benzoate (54 mg, 0.11 mmol) was dissolved in tetrahydrofuran (2.5 mL) and a solution of methyl magnesium bromide (0.81 mL, 1.4M) was added under a nitrogen atmosphere slowly at 0° C. It was stirred at 0° C. for 2 h, followed by stirring at room temperature overnight. Then, again a solution of methyl magnesium bromide (0.3 mL, 1.4M) was added at 0° C. It was continued stirring at 0° C. for 2 hours and quenched at the same temperature with saturated aqueous ammonium chloride solution and extracted twice with ethyl acetate. The combined ethyl acetate phases were washed with brine, dried over sodium sulfate and concentrated in vacuo. Purification by preparative HPLC (Chromatorex C-18 10 μm, 125×30 mm, acetonitrile/water+0.1% aqueous ammonia (32%)) led to 2-(2-chlorophenyl)-N-{4-[2-(2-hydroxypropan-2-yl)phenoxy]-3-sulfamoylphenyl}acetamide (22 mg, 0.0463, 42% yield, 95% purity).

LC-MS (Method B): Rt=1.06 min

MS (ESIneg): m/z=473 (M−H)⁺

¹H-NMR (400 MHz, DMSO-d₆) δ [ppm]: 1.50 (s, 6H), 3.83 (s, 2H), 5.30 (s, 1H), 6.77-6.85 (m, 2H), 7.10-7.19 (m, 1H), 7.22-7.36 (m, 3H), 7.36-7.48 (m, 4H), 7.65 (dd, 1H), 7.71 (dd, 1H), 8.19 (d, 1H), 10.44 (s, 1H).

### Example 276

### N-[4-(4-Chlorophenoxy)-3-sulfamoylphenyl]-2-(2,3-dihydro-1,4-benzodioxin-6-yl)acetamide

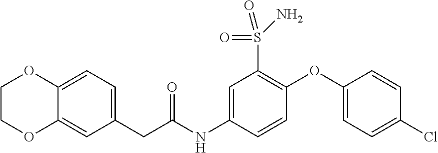 [SMILES: NS(=O)(=O)c1cc(NC(=O)Cc2ccc3c(c2)OCCO3)ccc1Oc1ccc(Cl)cc1]

According to GP3.1 and GP4 5-amino-2-(4-chlorophenoxy)-N-(2,4-dimethoxybenzyl)benzenesulfonamide (150 mg, 0.334 mmol) was reacted with 2,3-dihydro-1,4-benzodioxin-6-ylacetic acid (97 mg, 0.501 mmol) to N-[4-(4-chlorophenoxy)-3-sulfamoylphenyl]-2-(2,3-dihydro-1,4-benzodioxin-6-yl)acetamide. The pure compound was obtained after preparative HPLC (Chromatorex C-18 10 μm, 125×30 mm, acetonitrile/water+0.1% formic acid) (33 mg, 0.07 mmol, 21% yield over 2 steps, 95% purity).

LC-MS (Method A): Rt=1.09 min

MS (ESIneg): m/z=473 (M−H)⁺

¹H-NMR (DMSO-d₆) δ [ppm]: 3.49 (s, 2H), 4.21 (s, 4H), 6.72-6.85 (m, 3H), 6.96-7.08 (m, 3H), 7.35 (s, 2H), 7.39-7.47 (m, 2H), 7.73-7.81 (m, 1H), 8.16 (d, 1H), 10.35 (s, 1H).

### Example 277

### 2-(7-Chloro-2,3-dihydro-1,4-benzodioxin-6-yl)-N-[4-(3-chlorophenoxy)-3-sulfamoylphenyl]acetamide

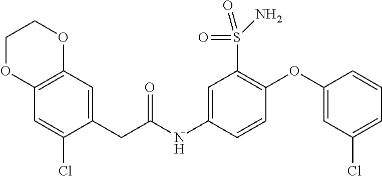 [SMILES: NS(=O)(=O)c1cc(NC(=O)Cc2cc3c(cc2Cl)OCCO3)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70 mg, 0.156 mmol) and (7-chloro-2,3-dihydro-1,4-benzodioxin-6-yl)acetic acid (39.2 mg, 0.172 mmol) were converted to 2-(7-chloro-2,3-dihydro-1,4-benzodioxin-6-yl)-N-[4-(3-chlorophenoxy)-3-sulfamoylphenyl]acetamide and were purified by preparative HPLC (Waters XBrigde C18 5μ 100×30 mm, acetonitrile/water+0.1% formic acid) (32.4 mg, 0.0636 mmol, 41% yield, 98% purity).

LC-MS (Method A): Rt=1.20 min

MS (ESIpos): m/z=509 (M+H)⁺

¹H-NMR (400 MHz, DMSO-d₆) δ [ppm]: 3.70 (s, 2H), 4.24 (s, 4H), 6.92-7.01 (m, 3H), 7.04-7.11 (m, 2H), 7.16-7.24 (m, 1H), 7.34-7.44 (m, 3H), 7.74-7.84 (m, 1H), 8.20 (d, 1H), 10.44 (s, 1H).

### Example 278

### 2-(5-Chloro-2,3-dihydro-1-benzofuran-4-yl)-N-[4-(3-chlorophenoxy)-3-sulfamoylphenyl]acetamide

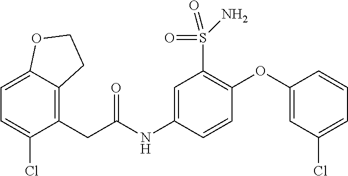 [SMILES: NS(=O)(=O)c1cc(NC(=O)Cc2c(Cl)ccc3c2CCO3)ccc1Oc1cccc(Cl)c1]

According to general procedures GP3.2 and GP4, purified 5-amino-2-(3-chlorophenoxy)-N-(2,4-dimethoxybenzyl)benzenesulfonamide (70 mg, 0.156 mmol) and (5-chloro-2,3-dihydro-1-benzofuran-4-yl)acetic acid (36.5 mg, 0.172 mmol) were converted to 2-(5-chloro-2,3-dihydro-1-benzofuran-4-yl)-N-[4-(3-chlorophenoxy)-3-sulfamoylphenyl]acetamide and were purified by preparative HPLC (Waters XBrigde C18 5μ 100×30 mm, acetonitrile/water+0.1% formic acid) (11 mg, 0.0223 mmol, 14% yield, 95% purity).

LC-MS (Method A): Rt=1.22 min

MS (ESIneg): m/z=491 (M−H)⁺

### Example 279

### 2-(2-Fluorophenyl)-N-[3-sulfamoyl-4-(tetrahydro-2H-pyran-4-ylmethoxy)phenyl]acetamide

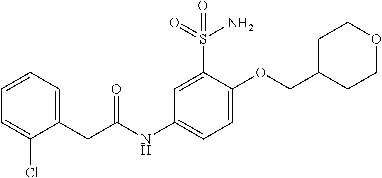 [SMILES: NS(=O)(=O)c1cc(NC(=O)Cc2ccccc2Cl)ccc1OCC1CCOCC1]

According to general procedures GP3.2 and GP4, 5-amino-N-(2,4-dimethoxybenzyl)-2-(tetrahydro-2H-pyran-4-ylmethoxy)benzenesulfonamide (54.0 mg, 0.124 mmol) and (2-fluorophenyl)acetic acid (22.9 mg, 0.148 mmol) were converted to 2-(2-fluorophenyl)-N-[3-sulfamoyl-4-(tetrahydro-2H-pyran-4-ylmethoxy)phenyl]acetamide and were purified by preparative HPLC (Waters XBrigde C18 5μ 100×30 mm, acetonitrile/water+0.1% formic acid) (27 mg, 0.0639 mmol, 52% yield, 98% purity).

LC-MS (Method A): Rt=0.94 min

MS (ESIpos): m/z=423 (M+H)⁺

¹H-NMR (500 MHz, DMSO-d₆) δ [ppm]: d [ppm]=1.27 (ddd, 2H), 1.67-1.75 (m, 2H), 2.09-2.20 (m, 1H), 3.27-3.35 (m, 2H), 3.69 (s, 2H), 3.86 (dd, 2H), 3.93 (d, 2H), 6.90 (s, 2H), 7.12-7.19 (m, 3H), 7.27-7.34 (m, 1H), 7.37 (td, 1H), 7.75 (dd, 1H), 8.01 (d, 1H), 10.29 (s, 1H).

### Example 280

### N-[3-Sulfamoyl-4-(tetrahydro-2H-pyran-4-ylmethoxy)phenyl]-2-[2-(trifluoromethyl)phenyl]acetamide

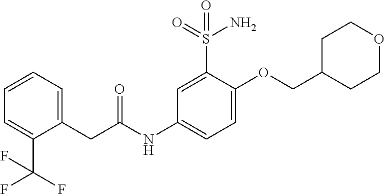 [SMILES: NS(=O)(=O)c1cc(NC(=O)Cc2ccccc2C(F)(F)F)ccc1OCC1CCOCC1]

According to general procedures GP3.2 and GP4, 5-amino-N-(2,4-dimethoxybenzyl)-2-(tetrahydro-2H-pyran-4-ylmethoxy)benzenesulfonamide (54.0 mg, 0.124 mmol) and [2-(trifluoromethyl)phenyl]acetic acid (30.3 mg, 0.148 mmol) were converted to N-[3-sulfamoyl-4-(tetrahydro-2H-pyran-4-ylmethoxy)phenyl]-2-[2-(trifluoromethyl)phenyl]acetamide and were purified by preparative HPLC (Waters XBrigde C18 5μ 100×30 mm, acetonitrile/water+0.1% formic acid) (35 mg, 0.0741 mmol, 60% yield, 98% purity).

LC-MS (Method A): Rt=1.05 min

MS (ESIpos): m/z=473 (M+H)⁺

¹H-NMR (500 MHz, DMSO-d₆) δ [ppm]: 1.27 (ddd, 2H), 1.69-1.74 (m, 2H), 2.09-2.19 (m, 1H), 3.27-3.34 (m, 2H), 3.83-3.88 (m, 2H), 3.88 (s, 2H), 3.92 (d, 2H), 6.90 (s, 2H), 7.15 (d, 1H), 7.45-7.53 (m, 2H), 7.64 (t, 1H), 7.70 (d, 1H), 7.73 (d, 1H), 8.00 (d, 1H), 10.29 (s, 1H).

### Example 281

### 2-[2-(Difluoromethyl)phenyl]-N-[3-sulfamoyl-4-(tetrahydro-2H-pyran-4-ylmethoxy)phenyl]acetamide

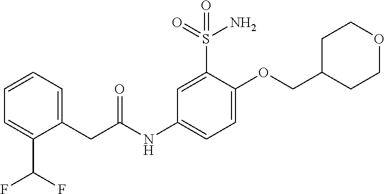 [SMILES: NS(=O)(=O)c1cc(NC(=O)Cc2ccccc2C(F)F)ccc1OCC1CCOCC1]

According to general procedures GP3.2 and GP4, 5-amino-N-(2,4-dimethoxybenzyl)-2-(tetrahydro-2H-pyran-4-ylmethoxy)benzenesulfonamide (55.0 mg, 0.126 mmol) and [2-(difluoromethyl)phenyl]acetic acid (28.1 mg, 0.151 mmol) were converted to 2-[2-(difluoromethyl)phenyl]-N-[3-sulfamoyl-4-(tetrahydro-2H-pyran-4-ylmethoxy)phenyl]acetamide and were purified by preparative HPLC (Waters XBrigde C18 5μ 100×30 mm, acetonitrile/water+0.1% formic acid) (30 mg, 0.0660 mmol, 52% yield, 98% purity).

LC-MS (Method A): Rt=1.00 min

MS (ESIpos): m/z=455 (M+H)⁺

¹H-NMR (400 MHz, DMSO-d₆) δ [ppm]: 1.26 (ddd, 2H), 1.71 (dd, 2H), 2.09-2.19 (m, 1H), 3.27-3.33 (m, 2H), 3.82-3.89 (m, 4H), 3.92 (d, 2H), 6.90 (s, 2H), 7.15 (d, 1H), 7.23 (t, 1H), 7.38-7.43 (m, 2H), 7.47-7.51 (m, 1H), 7.58 (d, 1H), 7.75 (dd, 1H), 7.99 (d, 1H), 10.29 (s, 1H).

### Example 282

### 2-(2-Chloro-4-fluorophenyl)-N-[3-sulfamoyl-4-(tetrahydro-2H-pyran-4-ylmethoxy)phenyl]acetamide

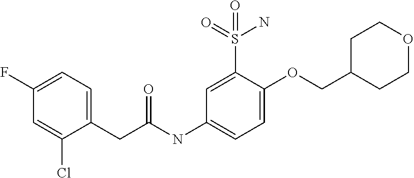 [SMILES: NS(=O)(=O)c1cc(NC(=O)Cc2ccc(F)cc2Cl)ccc1OCC1CCOCC1]

According to general procedures GP3.2 and GP4, 5-amino-N-(2,4-dimethoxybenzyl)-2-(tetrahydro-2H-pyran-4-ylmethoxy)benzenesulfonamide (55.0 mg, 0.126 mmol) and (2-chloro-4-fluorophenyl)acetic acid (28.5 mg, 0.151 mmol) were converted to 2-(2-chloro-4-fluorophenyl)-N-[3-sulfamoyl-4-(tetrahydro-2H-pyran-4-ylmethoxy)phenyl]acetamide and were purified by preparative HPLC (Waters XBrigde C18 5μ 100×30 mm, acetonitrile/water+0.1% formic acid) (11 mg, 0.0241 mmol, 19% yield, 97% purity).

LC-MS (Method A): Rt=1.02 min

MS (ESIpos): m/z=457 (M+H)⁺

¹H-NMR (400 MHz, DMSO-d₆) δ [ppm]: 1.27 (ddd, 2H), 1.70-1.76 (m, 2H), 2.09-2.22 (m, 1H), 3.28-3.37 (m, 2H), 3.80 (s, 2H), 3.84-3.91 (m, 2H), 3.94 (d, 2H), 6.92 (s, 2H), 7.17 (d, 1H), 7.22 (td, 1H), 7.42-7.50 (m, 2H), 7.76 (dd, 1H), 8.02 (d, 1H), 10.31 (s, 1H).

### Example 283

### 2-(2-Chlorophenyl)-N-(3-sulfamoyl-4-{[6-(trifluoromethyl)pyridin-3-yl]oxy}phenyl)-acetamide

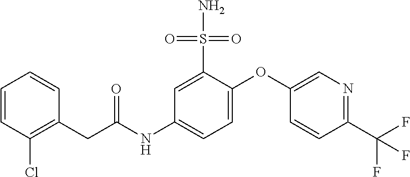 [SMILES: NS(=O)(=O)c1cc(NC(=O)Cc2ccccc2Cl)ccc1Oc1ccc(C(F)(F)F)nc1]

According to general procedures GP1.1, GP2.2, GP3.2 and GP4, 2-chloro-N-(2,4-dimethoxybenzyl)-5-nitrobenzenesulfonamide (500 mg, 1.29 mmol), 5-(trifluoromethyl)pyridin-2-ol (211 mg, 1.29 mmol) and (2-chlorophenyl)acetic acid (221 mg, 1.29 mmol) were converted without purification of intermediates to 2-(2-chlorophenyl)-N-(3-sulfamoyl-4-{[6-(trifluoromethyl)pyridin-3-yl]oxy}phenyl)-acetamide and were purified at the end by preparative HPLC (Waters XBridge C18 5μ 100×30 mm, acetonitrile/water+0.1% formic acid) (21 mg, 0.0432 mmol, 3% yield over 4 steps, 98% purity).

LC-MS (Method A): Rt=1.14 min

MS (ESIpos): m/z=486 (M+H)⁺

¹H-NMR (400 MHz, DMSO-d₆) δ [ppm]: 3.80 (s, 2H), 6.90 (d, 1H), 7.27-7.36 (m, 2H), 7.39-7.48 (m, 2H), 7.60 (dd, 1H), 7.66 (dd, 1H), 7.79 (d, 1H), 8.15 (d, 1H), 8.48 (d, 1H), 10.27 (s, 1H), 10.86 (br s, 1H), 10.91 (br s, 1H).

### Example 284

### 2-(2-Chlorophenyl)-N-(4-{[5-chloro-4-(trifluoromethyl)pyridin-2-yl]oxy}-3-sulfamoyl-phenyl)acetamide

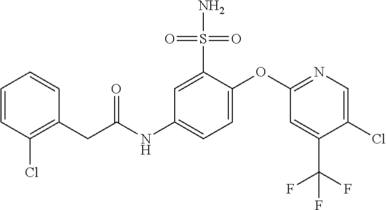 [SMILES: NS(=O)(=O)c1cc(NC(=O)Cc2ccccc2Cl)ccc1Oc1cc(C(F)(F)F)c(Cl)cn1]

According to general procedures GP1.1, GP2.2, GP3.2 and GP4, 2-chloro-N-(2,4-dimethoxybenzyl)-5-nitrobenzenesulfonamide (500 mg, 1.29 mmol), 5-chloro-4-(trifluoromethyl)pyridin-2-ol (255 mg, 1.29 mmol) and (2-chlorophenyl)acetic acid (221 mg, 1.29 mmol) were converted without purification of intermediates to 2-(2-chlorophenyl)-N-(4-{[5-chloro-4-(trifluoromethyl)pyridin-2-yl]oxy}-3-sulfamoyl-phenyl)acetamide and were purified at the end by preparative HPLC (Waters XBridge C18 5μ 100×30 mm, acetonitrile/water+0.1% formic acid) (25 mg, 0.0480 mmol, 4% yield over 4 steps, 96% purity).

LC-MS (Method A): Rt=1.25 min

MS (ESIpos): m/z=520 (M+H)⁺

¹H-NMR (400 MHz, DMSO-d₆) δ [ppm]: 3.81 (s, 2H), 6.86 (d, 1H), 7.26-7.53 (m, 5H), 7.67 (dd, 1H), 8.11 (d, 1H), 8.44 (s, 1H), 10.29 (s, 1H), 10.63 (br s, 1H), 11.44 (br s, 1H).

### Example 285

### N-[4-(3-Chlorophenoxy)-3-sulfamoylphenyl]-2-phenyl(²H₂)acetamide

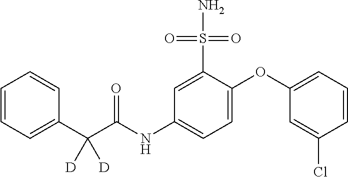 [SMILES: [2H]C([2H])(C(=O)Nc1ccc(Oc2cccc(Cl)c2)c(S(N)(=O)=O)c1)c1ccccc1]

According to general procedures GP3.2 and GP4, 5-amino-2-(3-chlorophenoxy)-N-(2,4-dimethoxybenzyl)benzenesulfonamide (100 mg, 0.223 mmol) and phenyl(²H₂)acetic acid (33.9 mg, 0.245 mmol) were converted to N-[4-(3-chlorophenoxy)-3-sulfamoylphenyl]-2-phenyl(²H₂)acetamide and were purified at the end by preparative HPLC (Waters XBrigde C18 5μ 100×30 mm, acetonitrile/water+0.1% formic acid) followed by another preparative HPLC (Chromatorex C-18 10 μm, 125×30 mm, acetonitrile/water+0.1% formic acid) (15 mg, 0.0358 mmol, 16% yield, 95% purity).

LC-MS (Method A): Rt=1.14 min

MS (ESIpos): m/z=418 (M+H)⁺

¹H-NMR (400 MHz, DMSO-d₆) δ [ppm]: 6.97 (ddd, 1H), 7.06-7.11 (m, 2H), 7.19 (ddd, 1H), 7.22-7.43 (m, 8H), 7.82 (dd, 1H), 8.20 (d, 1H), 10.49 (s, 1H).

### Example 286

### N-{4-[(6-Chloro-5-fluoropyridin-3-yl)oxy]-3-sulfamoylphenyl}-2-(2-chlorophenyl)-acetamide

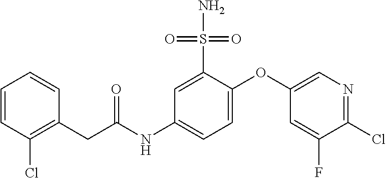 [SMILES: NS(=O)(=O)c1cc(NC(=O)Cc2ccccc2Cl)ccc1Oc1cnc(Cl)c(F)c1]

According to general procedures GP1.1, GP2.2, GP3.2 and GP4, 2-chloro-N-(2,4-dimethoxybenzyl)-5-nitrobenzenesulfonamide (500 mg, 1.29 mmol), 6-chloro-5-fluoropyridin-3-ol (191 mg, 1.29 mmol) and (2-chlorophenyl)acetic acid (221 mg, 1.29 mmol) were converted without purification of intermediates to N-{4-[(6-chloro-5-fluoropyridin-3-yl)oxy]-3-sulfamoylphenyl}-2-(2-chlorophenyl)-acetamide and were purified at the end by preparative HPLC (Waters XBridge C18 5μ 100×30 mm, acetonitrile/water+0.1% formic acid) (15 mg, 0.0319 mmol, 2% yield over 4 steps, 97% purity).

LC-MS (Method A): Rt=1.13 min

MS (ESIpos): m/z=470 (M+H)⁺

¹H-NMR (400 MHz, DMSO-d₆) δ [ppm]: 3.80 (s, 2H), 6.91 (d, 1H), 7.27-7.35 (m, 2H), 7.39-7.48 (m, 2H), 7.52 (dd, 1H), 7.62 (dd, 1H), 8.02 (d, 1H), 8.10 (d, 1H), 10.26 (s, 1H), 10.82 (br s, 2H).

### Example 287

### 2-(2-Chlorophenyl)-N-{4-[(4,4-difluoro-1-hydroxycyclohexyl)methoxy]-3-sulfamoyl-phenyl}acetamide

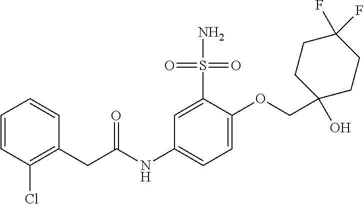 [SMILES: NS(=O)(=O)c1cc(NC(=O)Cc2ccccc2Cl)ccc1OCC1(O)CCC(F)(F)CC1]

According to general procedures GP1.2 (but with tetrahydrofuran as solvent and 3.5 eq sodium hydride; 10 min at 0° C., then 2 h at room temperature), GP2.1 (but with methanol as solvent), GP3.2 (but 1.5 eq acid, 1.5 eq HATU and 3 eq base) and GP4 (4 mL DCM and 2 mL TFA), 2-chloro-N-(2,4-dimethoxybenzyl)-5-nitrobenzenesulfonamide (500 mg, 1.29 mmol), 4,4-difluoro-1-(hydroxymethyl)cyclohexanol (322 mg, 1.94 mmol) and (2-chlorophenyl)acetic acid (333 mg, 1.95 mmol) were converted without purification of intermediates to 2-(2-chlorophenyl)-N-{4-[(4,4-difluoro-1-hydroxycyclohexyl)methoxy]-3-sulfamoyl-phenyl}acetamide and were purified at the end by preparative HPLC (Waters XBridge C18 5μ 100×30 mm, acetonitrile/water+0.1% formic acid) (265 mg, 0.542 mmol, 42% yield over 4 steps, 99% purity).

LC-MS (Method A): Rt=1.08 min

MS (ESIneg): m/z=487 (M−H)⁻

¹H-NMR (400 MHz, DMSO-d₆) δ [ppm]: 1.59-1.70 (m, 2H), 1.81-2.17 (m, 6H), 3.82 (s, 2H), 4.00 (s, 2H), 5.17 (s, 1H), 7.08 (s, 2H), 7.19 (d, 1H), 7.28-7.35 (m, 2H), 7.40-7.48 (m, 2H), 7.78 (dd, 1H), 8.03 (d, 1H), 10.34 (s, 1H).

### Example 288

### 2-(2-Chlorophenyl)-N-{4-[(1-hydroxycyclohexyl)methoxy]-3-sulfamoylphenyl}-acetamide

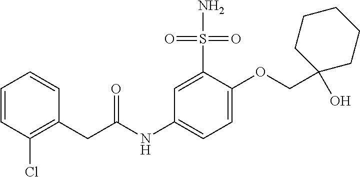 [SMILES: NS(=O)(=O)c1cc(NC(=O)Cc2ccccc2Cl)ccc1OCC1(O)CCCCC1]

According to general procedures GP1.2 (but with tetrahydrofuran as solvent and 3.5 eq sodium hydride; 10 min at 0° C., then 2 h at room temperature), GP2.1 (but with methanol as solvent), GP3.2 (but 1.5 eq acid, 1.5 eq HATU and 3 eq base) and GP4 (4 mL DCM and 2 mL TFA), 2-chloro-N-(2,4-dimethoxybenzyl)-5-nitrobenzenesulfonamide (500 mg, 1.29 mmol), 1-(hydroxymethyl)cyclohexanol (252 mg, 1.94 mmol) and (2-chlorophenyl)acetic acid (335 mg, 1.96 mmol) were converted without purification of intermediates to 2-(2-chlorophenyl)-N-{4-[(1-hydroxycyclohexyl)methoxy]-3-sulfamoyl-phenyl}-acetamide and were purified at the end by preparative HPLC (Waters XBridge C18 5μ 100×30 mm, acetonitrile/water+0.1% formic acid) (241 mg, 0.533 mmol, 41% yield over 4 steps, 99% purity).

LC-MS (Method A): Rt=1.10 min

MS (ESIneg): m/z=451 (M−H)⁻

¹H-NMR (400 MHz, DMSO-d₆) δ [ppm]: 1.22-1.34 (m, 1H), 1.38-1.67 (m, 9H), 3.82 (s, 2H), 3.93 (s, 2H), 4.85 (s, 1H), 7.10 (s, 2H), 7.19 (d, 1H), 7.28-7.37 (m, 2H), 7.39-7.48 (m, 2H), 7.77 (dd, 1H), 8.01 (d, 1H), 10.33 (s, 1H).

### Example 289

### N-[4-(3-Chlorophenoxy)-3-fluoro-5-sulfamoylphenyl]-2-(2-fluorophenyl)acetamide

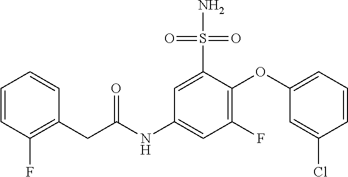 [SMILES: NS(=O)(=O)c1cc(NC(=O)Cc2ccccc2F)cc(F)c1Oc1cccc(Cl)c1]

According to general procedures GP3.2 and GP4, crude 5-amino-2-(3-chlorophenoxy)-N-(2,4-dimethoxybenzyl)-3-fluorobenzenesulfonamide (75.0 mg, 0.161 mmol) and (2-fluorophenyl)acetic acid (27.2 mg, 0.177 mmol) were converted to N-[4-(3-chlorophenoxy)-3-fluoro-5-sulfamoylphenyl]-2-(2-fluorophenyl)acetamide and were purified by preparative HPLC (Waters XBridge C18 5μ 100×30 mm, acetonitrile/water+0.1% formic acid) (9 mg, 0.0199 mmol, 95% purity).

LC-MS (Method A): Rt=1.30 min

MS (ESIpos): m/z=453 (M+H)⁺

¹H-NMR (400 MHz, DMSO-d₆) δ [ppm]: 3.65 (s, 2H), 6.03 (s, 2H), 7.02-7.09 (m, 1H), 7.13-7.20 (m, 2H), 7.24-7.27 (m, 1H), 7.28-7.38 (m, 2H), 7.42-7.47 (m, 2H), 7.52-7.57 (m, 1H), 7.77 (dd, 1H), 10.27 (s, 1H).

### Example 290

### N-[4-(3-Chlorophenoxy)-3-fluoro-5-sulfamoylphenyl]-2-[2-(difluoromethyl)-phenyl]acetamide

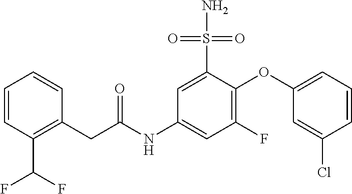 [SMILES: NS(=O)(=O)c1cc(NC(=O)Cc2ccccc2C(F)F)cc(F)c1Oc1cccc(Cl)c1]

According to general procedures GP3.2 and GP4, crude 5-amino-2-(3-chlorophenoxy)-N-(2,4-dimethoxybenzyl)-3-fluorobenzenesulfonamide (75.0 mg, 0.161 mmol) and [2-(difluoromethyl)phenyl]acetic acid (32.9 mg, 0.177 mmol) were converted to N-[4-(3-chlorophenoxy)-3-fluoro-5-sulfamoylphenyl]-2-[2-(difluoromethyl)-phenyl]acetamide and were purified by preparative HPLC (Waters XBridge C18 5μ 100×30 mm, acetonitrile/water+0.1% formic acid) (5 mg, 0.0103 mmol, 90% purity).

LC-MS (Method A): Rt=1.33 min

MS (ESIpos): m/z=458 (M+H)⁺

¹H-NMR (400 MHz, DMSO-d₆) δ [ppm]: 3.79 (s, 2H), 6.04 (s, 2H), 7.04-7.08 (m, 1H), 7.20 (t, 1H), 7.24-7.27 (m, 1H), 7.36-7.54 (m, 6H), 7.58 (d, 1H), 7.76 (dd, 1H), 10.27 (s, 1H).

### Example 291

### N-[4-(3-Chlorophenoxy)-3-fluoro-5-sulfamoylphenyl]-2-(2-chlorophenyl)acetamide

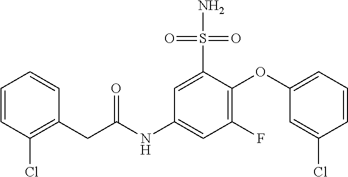 [SMILES: NS(=O)(=O)c1cc(NC(=O)Cc2ccccc2Cl)cc(F)c1Oc1cccc(Cl)c1]

According to general procedures GP3.2 and GP4, crude 5-amino-2-(3-chlorophenoxy)-N-(2,4-dimethoxybenzyl)-3-fluorobenzenesulfonamide (150 mg, 0.321 mmol) and (2-chlorophenyl)acetic acid (60.3 mg, 0.353 mmol) were converted to N-[4-(3-chlorophenoxy)-3-fluoro-5-sulfamoylphenyl]-2-(2-chlorophenyl)acetamide and were purified by preparative HPLC (Waters XBridge C18 5μ 100×30 mm, acetonitrile/water+0.1% formic acid) (11 mg, 0.0234 mmol, 95% purity).

LC-MS (Method A): Rt=1.34 min

MS (ESIpos): m/z=469 (M+H)⁺

¹H-NMR (400 MHz, DMSO-d₆) δ [ppm]: 3.76 (s, 2H), 6.03 (br s, 2H), 7.03-7.09 (m, 1H), 7.24-7.27 (m, 1H), 7.28-7.32 (m, 2H), 7.37-7.41 (m, 1H), 7.42-7.47 (m, 3H), 7.52-7.56 (m, 1H), 7.76 (dd, 1H), 10.27 (s, 1H).

### Example 292

### 2-(2-Chloro-5-fluorophenyl)-N-[4-(3-chlorophenoxy)-3-fluoro-5-sulfamoyl-phenyl]acetamide

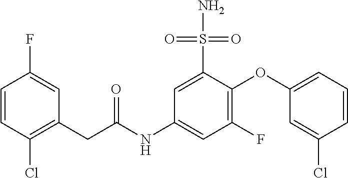 [SMILES: NS(=O)(=O)c1cc(NC(=O)Cc2cc(F)ccc2Cl)cc(F)c1Oc1cccc(Cl)c1]

According to general procedures GP3.2 and GP4, crude 5-amino-2-(3-chlorophenoxy)-N-(2,4-dimethoxybenzyl)-3-fluorobenzenesulfonamide (75.0 mg, 0.161 mmol) and (2-chloro-5-fluorophenyl)acetic acid (33.3 mg, 0.177 mmol) were converted to 2-(2-chloro-5-fluorophenyl)-N-[4-(3-chlorophenoxy)-3-fluoro-5-sulfamoyl-phenyl]acetamide and were purified by preparative HPLC (Waters XBridge C18 5μ 100×30 mm, acetonitrile/water+0.1% formic acid) (15 mg, 0.0328 mmol, 90% purity).

LC-MS (Method A): Rt=1.35 min

MS (ESIpos): m/z=487 (M+H)⁺

¹H-NMR (400 MHz, DMSO-d₆) δ [ppm]: 3.77 (s, 2H), 6.03 (s, 2H), 7.03-7.08 (m, 1H), 7.18 (td, 1H), 7.23-7.26 (m, 1H), 7.30 (dd, 1H), 7.42-7.46 (m, 2H), 7.48 (dd, 1H), 7.51-7.54 (m, 1H), 7.74 (dd, 1H), 10.28 (s, 1H).

### Example 293

### N-[6-(3-Chlorophenoxy)-5-sulfamoylpyridin-3-yl]-2-(2-fluorophenyl)acetamide

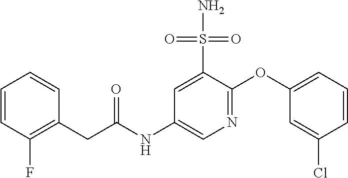 [SMILES: NS(=O)(=O)c1cc(NC(=O)Cc2ccccc2F)cnc1Oc1cccc(Cl)c1]

5-Amino-2-(3-chlorophenoxy)pyridine-3-sulfonamide (56 mg, 0.187 mmol) was dissolved in DMF (0.5 mL) followed by addition of (2-fluorophenyl)acetic acid (34.6 mg, 0.224 mmol), HATU (114 mg, 0.299 mmol) and N,N-diisopropylethylamine (121 mg, 0.934 mmol). After stirring at 50° C. for 4 h it was concentrated in vacuo, extracted with ethyl acetate/water and the organic phase was dried over sodium sulfate and concentrated in vacuo again. Purification by preparative HPLC (Waters XBridge C18 5μ 100×30 mm, acetonitrile/water+0.2% aqueous ammonia (32%)) gave N-[6-(3-chlorophenoxy)-5-sulfamoylpyridin-3-yl]-2-(2-fluorophenyl)acetamide (21 mg, 0.0482 mmol, 26% yield, 98% purity).

LC-MS (Method B): Rt=0.98 min

MS (ESIpos): m/z=436 (M+H)⁺

¹H-NMR (400 MHz, DMSO-d₆) δ [ppm]: 3.77 (s, 2H), 7.14-7.22 (m, 3H), 7.27-7.37 (m, 3H), 7.41 (td, 1H), 7.46 (t, 1H), 7.59 (brs, 2H), 8.47 (d, 1H), 8.61 (d, 1H), 10.66 (s, 1H).

### Example 294

### N-[6-(3-Chlorophenoxy)-5-sulfamoylpyridin-3-yl]-2-[2-(trifluoromethyl)phenyl]-acetamide

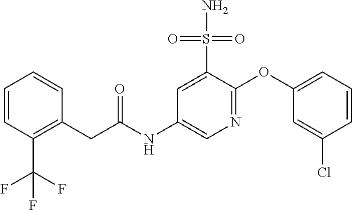 [SMILES: NS(=O)(=O)c1cc(NC(=O)Cc2ccccc2C(F)(F)F)cnc1Oc1cccc(Cl)c1]

5-Amino-2-(3-chlorophenoxy)pyridine-3-sulfonamide (56 mg, 0.187 mmol) was dissolved in DMF (2 mL) followed by addition of [2-(trifluoromethyl)phenyl]acetic acid (42.0 mg, 0.206 mmol), HATU (114 mg, 0.299 mmol) and N,N-diisopropylethylamine (121 mg, 0.934 mmol). After stirring at 50° C. for 4 h it was concentrated in vacuo, extracted with ethyl acetate/water and the organic phase was dried over sodium sulfate and concentrated in vacuo again. Purification by preparative HPLC (Waters XBridge C18 5μ 100×30 mm, acetonitrile/water+0.2% aqueous ammonia (32%)) gave N-[6-(3-chlorophenoxy)-5-sulfamoylpyridin-3-yl]-2-[2-(trifluoromethyl)phenyl]-acetamide (18 mg, 0.0370 mmol, 20% yield, 98% purity).

LC-MS (Method B): Rt=1.11 min

MS (ESIpos): m/z=486 (M+H)⁺

¹H-NMR (400 MHz, DMSO-d₆) δ [ppm]: 3.96 (s, 2H), 7.17 (ddd, 1H), 7.28 (t, 1H), 7.31 (ddd, 1H), 7.42-7.58 (m, 3H), 7.62-7.70 (m, 3H), 7.73 (d, 1H), 8.46 (d, 1H), 8.61 (d, 1H), 10.67 (s, 1H).

### Example 295

### N-[6-(3-Chlorophenoxy)-5-sulfamoylpyridin-3-yl]-2-[2-(difluoromethyl)phenyl]-acetamide

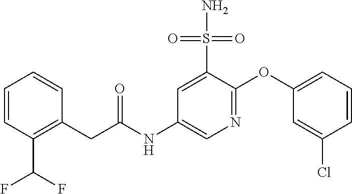 [SMILES: NS(=O)(=O)c1cc(NC(=O)Cc2ccccc2C(F)F)cnc1Oc1cccc(Cl)c1]

5-Amino-2-(3-chlorophenoxy)pyridine-3-sulfonamide (56 mg, 0.187 mmol) was dissolved in DMF (2 mL) followed by addition of [2-(difluoromethyl)phenyl]acetic acid (38.3 mg, 0.206 mmol), HATU (114 mg, 0.299 mmol) and N,N-diisopropylethylamine (121 mg, 0.934 mmol). After stirring at 50° C. for 4 h it was concentrated in vacuo, extracted with ethyl acetate/water and the organic phase was dried over sodium sulfate and concentrated in vacuo again. Purification by preparative HPLC (Waters XBridge C18 5μ 100×30 mm, acetonitrile/water+0.2% aqueous ammonia (32%)) gave N-[6-(3-Chlorophenoxy)-5-sulfamoylpyridin-3-yl]-2-[2-(difluoromethyl)phenyl]-acetamide (7 mg, 0.0153 mmol, 8% yield, 95% purity).

LC-MS (Method B): Rt=1.14 min

MS (ESIpos): m/z=468 (M+H)⁺

¹H-NMR (400 MHz, DMSO-d₆) δ [ppm]: 3.91 (s, 2H), 7.16 (ddd, 1H), 7.22 (t, 1H), 7.28 (t, 1H), 7.31 (ddd, 1H), 7.40-7.56 (m, 4H), 7.60 (d, 1H), 7.64 (s, 2H), 8.46 (d, 1H), 8.61 (d, 1H), 10.65 (s, 1H).

### Example 296

### 2-(2-Chloro-5-fluorophenyl)-N-[6-(3-chlorophenoxy)-5-sulfamoylpyridin-3-yl]-acetamide

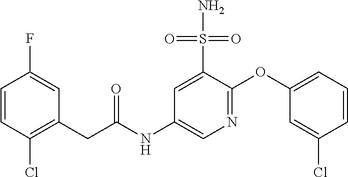 [SMILES: NS(=O)(=O)c1cc(NC(=O)Cc2cc(F)ccc2Cl)cnc1Oc1cccc(Cl)c1]

5-Amino-2-(3-chlorophenoxy)pyridine-3-sulfonamide (56 mg, 0.187 mmol) was dissolved in DMF (2 mL) followed by addition of (2-chloro-5-fluorophenyl)acetic acid (38.8 mg, 0.206 mmol), HATU (114 mg, 0.299 mmol) and N,N-diisopropylethylamine (121 mg, 0.934 mmol). After stirring at 50° C. for 4 h it was concentrated in vacuo, extracted with ethyl acetate/water and the organic phase was dried over sodium sulfate and concentrated in vacuo again. Purification by preparative HPLC (Waters XBridge C18 5μ 100×30 mm, acetonitrile/water+0.2% aqueous ammonia (32%)) gave 2-(2-chloro-5-fluorophenyl)-N-[6-(3-chlorophenoxy)-5-sulfamoylpyridin-3-yl]-acetamide (2 mg, 0.00425 mmol, 3% yield, 94% purity).

LC-MS (Method B): Rt=1.01 min

MS (ESIpos): m/z=470 (M+H)⁺

¹H-NMR (400 MHz, DMSO-d₆) δ [ppm]: 3.89 (s, 2H), 7.15-7.24 (m, 2H), 7.28 (t, 1H), 7.22 (ddd, 1H), 7.37 (dd, 1H), 7.43-7.54 (m, 2H), 7.65 (s, 2H), 8.47 (d, 1H), 8.62 (d, 1H), 10.70 (s, 1H).

### Biological Assays

The following assays can be used to illustrate the commercial utility of the compounds according to the present invention.

Examples were tested in selected biological assays one or more times. When tested more than once, data are reported as either average (avg) values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

When no meaningful calculation of average values or median values is possible due to the existence of measurement values falling outside the detection range of the assay (indicated by < or > in the tables below) all individual measurement values are indicated. Examples were synthesized one or more times. When synthesized more than once, data from biological assays represent average values or median values calculated utilizing data sets obtained from testing of one or more synthetic batch.

### Cellular In Vitro Assays for Determination of P2X4 Receptor Activity

### Assay A: Human P2X4 Receptor Cell Line

A fluorescent imaging plate reader (FLEX/FLIPR station; Molecular Devices) was used to monitor intracellular calcium levels using the calcium-chelating dye Fluo-8 (Molecular Probes). The excitation and emission wavelengths used to monitor fluorescence were 470-495 nm and 515-575 nm, respectively. Cells expressing purinergic receptors human P2X4 (HEK human P2X4: PerkinElmer, Product No.: AX-015-PCF) were plated at a density of 20,000 cells/well in poly-D-lysine-coated 384-well plates approximately 20 hours before beginning the assay. On the day of the assay, the medium was removed and 30 μl of dye buffer (Hank's balanced salt solution, 10 mM HEPES, 1.8 mM CaCl₂), 1 mM MgCl₂, 2 mM probenecid, 5 mM D-glucose monohydrate, 5 μM Fluo-8, pH=7.4) was added for 30 min at 370 C and 5% CO₂. The antagonist in probenecid buffer (Hank's balanced salt solution, 10 mM HEPES, 1.8 mM CaCl₂, 1 mM MgCl₂, 2 mM probenecid, 5 mM D-glucose monohydrate, pH=7.4) was added in a volume of 10 μl and allowed to incubate for 30 min at room temperature. The final assay DMSO concentration is 0.5%. The agonist, Bz-ATP, is added in a volume of 10 μl at a concentration representing the EC₈₀ value. The fluorescence was measured for an interval of 120 sec at 2 sec intervals and analyzed based on the increase in peak relative fluorescence units (RFU) compared to the basal fluorescence. Peak fluorescence was used to determine the response to agonist obtained at each concentration of antagonist by the following equation:

% Response=100*(RFU(drug)−RFU(control))/(RFU(DMSO)−RFU(control))

The compounds were tested in triplicates per plate and mean values were plotted in Excel XLFit to determine IC₅₀ values, percentage of maximal inhibition and the Hill coefficients.

| | human P2X4 | human P2X4 |
|---|---|---|
| | Assay A | Assay A |
| Example Number | avg IC₅₀ [nM] | avg Efficacy [%] |
| 002 | 29 | 96 |
| 004 | 149 | 90 |
| 005 | 77 | 94 |
| 006 | 99 | 87 |
| 007 | 16 | 93 |
| 008 | 44 | 88 |
| 009 | 93 | 99 |
| 010 | 27 | 90 |
| 011 | 41 | 85 |
| 012 | 20 | 70 |
| 013 | 441 | 93 |
| 014 | 10 | 85 |
| 015 | 58 | 71 |
| 016 | 96 | 81 |
| 017 | 61 | 86 |
| 018 | 9 | 93 |
| 020 | 67 | 90 |
| 022 | 8 | 87 |
| 024 | 114 | 82 |
| 026 | 21 | 95 |
| 028 | 193 | 75 |
| 029 | 27 | 85 |
| 031 | 24 | 76 |
| 032 | 14 | 100 |
| 036 | 106 | 81 |
| 037 | 63 | 70 |
| 039 | 7 | 89 |
| 042 | 241 | 89 |
| 043 | 515 | 85 |
| 047 | 7 | 69 |
| 050 | 5 | 94 |
| 051 | 20 | 87 |
| 052 | 45 | 85 |
| 053 | 197 | 90 |
| 054 | 68 | 94 |
| 059 | 45 | 85 |
| 061 | 53 | 88 |
| 062 | 560 | 95 |
| 063 | 90 | 82 |
| 064 | 30 | 79 |
| 065 | 13 | 81 |
| 066 | >25000 | 51 |
| 068 | 75 | 96 |
| 077 | 8341 | 54 |
| 079 | 2528 | 54 |
| 080 | 44 | 65 |
| 081 | 65 | 43 |
| 082 | >25000 | 31 |
| 083 | >25000 | 39 |
| 085 | 269 | 86 |
| 086 | 24 | 90 |
| 089 | 102 | 85 |
| 091 | 27 | 75 |
| 095 | 137 | 64 |
| 096 | 78 | 72 |
| 097 | 119 | 84 |
| 098 | 7 | 95 |
| 099 | 168 | 77 |
| 100 | 9 | 81 |
| 101 | 38 | 97 |
| 102 | 68 | 85 |
| 103 | 208 | 78 |
| 104 | 15 | 78 |
| 107 | 201 | 86 |
| 110 | 254 | 79 |
| 113 | 173 | 88 |
| 116 | 1718 | 87 |
| 117 | 43 | 94 |
| 120 | 65 | 83 |
| 122 | 26 | 83 |
| 128 | 184 | 77 |
| 129 | 1174 | 83 |
| 130 | 979 | 91 |
| 131 | 284 | 77 |
| 132 | 541 | 72 |
| 133 | 375 | 75 |
| 134 | 688 | 83 |
| 135 | 631 | 75 |
| 136 | 10 | 90 |
| 137 | 35 | 88 |
| 138 | 4 | 67 |
| 139 | 26 | 82 |
| 140 | 8 | 103 |
| 141 | 2 | 96 |
| 142 | 43 | 90 |
| 143 | 9 | 72 |
| 144 | 86 | 93 |
| 148A | 74 | 92 |
| 149 | 45 | 81 |
| 150 | 26 | 96 |
| 151 | 143 | 88 |
| 152 | >25000 | 47 |
| 155 | 19 | 87 |
| 156 | 12 | 81 |
| 157 | 5 | 67 |
| 158 | 784 | 91 |
| 160 | 10 | 71 |
| 163 | 84 | 68 |
| 164 | 139 | 90 |
| 165 | 162 | 59 |
| 166 | 13 | 77 |
| 167 | 37 | 87 |
| 168 | 86 | 90 |
| 169 | 274 | 74 |
| 173 | 163 | 100 |
| 174 | 1681 | 74 |
| 175 | 1300 | 52 |
| 176 | 459 | 82 |
| 177 | 265 | 80 |
| 178 | 392 | 76 |
| 179 | 317 | 69 |
| 180 | 345 | 76 |
| 182 | 10 | 84 |
| 183 | 52 | 81 |
| 187 | 92 | 74 |
| 191 | 50 | 85 |
| 192 | 2 | 86 |
| 196 | 134 | 82 |
| 200 | 39 | 86 |
| 202 | 121 | 72 |
| 207 | 473 | 60 |
| 208 | 38 | 87 |
| 214 | 76 | 80 |
| 224 | 345 | 78 |
| 229 | 472 | 63 |
| 231 | 529 | 62 |
| 232 | 215 | 57 |
| 233 | 185 | 70 |
| 234 | 562 | 73 |
| 235 | 473 | 79 |
| 244 | 211 | 74 |
| 256 | 19 | 75 |
| 257 | 355 | 85 |
| 258 | 779 | 76 |
| 259 | 145 | 76 |
| 260 | 2487 | 90 |
| 261 | 20 | 89 |
| 262 | 42 | 54 |
| 263 | 35 | 95 |
| 264 | 376 | 62 |
| 265 | 416 | 74 |
| 266 | 63 | 89 |
| 267 | 4440 | 76 |
| 268 | 258 | 77 |
| 269 | >25000 | 37 |
| 270 | 104 | 94 |
| 271 | 81 | 84 |
| 272 | 19 | 73 |
| 273 | 18 | 78 |
| 274 | 692 | 104 |
| 275 | 242 | 64 |
| 278 | 139 | 82 |
| 279 | 597 | 61 |
| 280 | 88 | 73 |
| 281 | 29 | 67 |
| 282 | 173 | 82 |
| 283 | 8051 | 62 |
| 284 | 10930 | 82 |
| 285 | 98 | 73 |
| 286 | 237 | 55 |
| 287 | 187 | 58 |
| 288 | 988 | 57 |
| 289 | >25000 | 34 |
| 290 | 15956 | 66 |
| 291 | 10500, >25000 | 57 |
| 292 | 15136 | 69 |
| 293 | 72 | 67 |
| 294 | 107 | 84 |
| 295 | 155 | 85 |
| 296 | 59 | 78 |

### Assay B: Human and Mouse P2X4 Receptor Cell Line

The identification of agonists and antagonists of the P2X4 receptor from humans and rats also the quantification of the activity of the compounds of the invention was performed by use of recombinant cell lines. These cell lines derive originally from a human embryonic kidney cell line (HEK 293, ATCC: American Type Culture Collection, Manassas, Va. 20108, USA). The test cell lines constitutively express the mouse or human P2X4 receptors. Stimulation of the receptors with an agonist leads to a conformational change of P2X4 and influx of extracellular calcium ions through the ion channel. The cytoplasmatic calcium transient is detected via the calcium sensitive dye Fluo8 for the mouse P2×4 cell line or in case of the human P2×4 cell line a stably expressed calcium-sensitive photoprotein, mitochondrial photina, which, after reconstitution with the cofactor coelenterazine, emits light in dependence of calcium binding [Bovolenta S, Foti M, Lohmer S, Corazza S., J Biomol Screen. 2007 August; 12(5):694-704]. The strength of Fluo8 fluorescence signal or photina luminescence signal corresponds to the level of receptor activation. An inhibitor would decrease the signal depending on its potency and concentration. Fluorescence was measured by use of the FLIPR (Molecular Devices), the bioluminescence was detected using a suitable luminometer [Milligan G, Marshall F, Rees S, Trends in Pharmacological Sciences 17, 235-237 (1996)].

### Test Procedure for Human P2X4 Receptor Cell Line

On the day before the assay, the cells (HEK human P2X4: PerkinElmer, Product No.: AX-015-PCF) are plated out in culture medium (DMEM high glucose, 2% FCS, 1% MEM non-essential amino acids, 10 mM HEPES, 5 μg/ml Coelenterazine) in 384-well poly-D-lysine coated microtiter plates and kept in a cell incubator (96% humidity, 5% v/v CO₂, 30° C.). On the day of the assay, the test substances in various concentrations are placed for 10 minutes in the wells of the microtiter plate before the agonist Bz-ATP at EC₅₀ concentration is added. The resulting light signal is measured immediately in the luminometer.

### Test Procedure for Mouse P2X4 Receptor Cell Line

On the day before the assay, the cells (HEK mouse P2X4: Axxam SpA (www.axxam.com)) are plated out in culture medium (DMEM high glucose, 10% FCS, 1% MEM non-essential amino acids, 10 mM HEPES) in 384-well poly-D-lysine coated microtiter plates and kept in a cell incubator (96% humidity, 5% v/v CO₂, 37° C.). On the day of the assay medium is exchanged by Fluo8 containing buffer for 30 minutes. In the fluorescence reader (FLIPR) test substances in various concentrations are added for 10 minutes. After baseline measurement the agonist Bz-ATP at EC₅₀ concentration is applied and fluorescence is measured immediately.

| | human P2X4 | human P2X4 | mouse P2X4 | mouse P2X4 |
|---|---|---|---|---|
| Example | Assay B | Assay B | Assay B | Assay B |
| Number | avg IC₅₀ [nM] | avg Efficacy [%] | avg IC₅₀ [nM] | avg Efficacy [%] |
| 001 | 7720 | 61 | | |
| 002 | 79 | 81 | | |
| 003 | 6340 | 50 | | |
| 004 | 95 | 68 | | |
| 005 | 90 | 89 | | |
| 006 | 73 | 108 | | |
| 007 | 122 | 104 | | |
| 008 | 73 | 91 | | |
| 009 | 98 | 70 | | |
| 010 | 73 | 76 | | |
| 011 | 43 | 67 | | |
| 012 | 43 | 56 | 1240 | 44 |
| 013 | 429 | 71 | >16000 | 31 |
| 014 | 49 | 70 | >16000 | 25 |
| 015 | 25 | 80 | 149 | 45 |
| 016 | 112 | 72 | | |
| 017 | 55 | 65 | 805 | 44 |
| 018 | 47 | 70 | | |
| 019 | 1419 | 80 | | |
| 020 | 152 | 96 | | |
| 021 | 3865 | 60 | | |
| 022 | 97 | 54 | | |
| 023 | 166 | 73 | | |
| 024 | 32 | 57 | | |
| 025 | >16000 | 50 | | |
| 026 | 82 | 71 | | |
| 027 | 776 | 99 | | |
| 028 | 718 | 79 | | |
| 029 | 102 | 71 | 337 | 50 |
| 030 | 5075 | 99 | | |
| 031 | 122 | 71 | | |
| 032 | 57 | 82 | | |
| 033 | 6750 | 75 | | |
| 034 | 172 | 57 | | |
| 035 | 3575 | 60 | | |
| 036 | 68 | 79 | >50000 | 15 |
| 037 | 188 | 56 | | |
| 038 | 777 | 74 | | |
| 039 | 140 | 65 | | |
| 040 | 238 | 64 | | |
| 041 | 3385 | 72 | | |
| 042 | 460 | 90 | >16000, >50000, | 54 |
| | | | >50000, >50000 | |
| 043 | 671 | 68 | | |
| 044 | 2630 | 69 | | |
| 045 | 176 | 71 | | |
| 046 | 841 | 75 | | |
| 047 | 862 | 57 | | |
| 048 | 11640 | 73 | | |
| 049 | 773 | 92 | | |
| 050 | 112 | 87 | | |
| 051 | 124 | 83 | | |
| 052 | 625 | 66 | | |
| 053 | 859 | 84 | | |
| 054 | 578 | 84 | | |
| 055 | 1160 | 71 | | |
| 056 | >50000 | 15 | | |
| 057 | 1595 | 65 | | |
| 058 | 3176 | 73 | | |
| 059 | 75 | 70 | | |
| 060 | 271, >50000 | 34 | | |
| 061 | 147 | 72 | | |
| 062 | 455 | 70 | | |
| 063 | 221 | 70 | | |
| 064 | 150 | 67 | | |
| 065 | 130 | 70 | | |
| 066 | 129 | 46 | | |
| 067 | 241 | 72 | | |
| 068 | 499 | 68 | | |
| 069 | 2676 | 71 | | |
| 070 | 1095 | 70 | | |
| 071 | 2090 | 73 | | |
| 072 | 2955 | 63 | | |
| 073 | 3365 | 65 | | |
| 074 | 609 | 52 | | |
| 075 | 2230 | 77 | | |
| 076 | 574 | 74 | | |
| 077 | 201 | 53 | | |
| 078 | 706 | 86 | | |
| 079 | >16000 | 40 | | |
| 080 | 140 | 51 | | |
| 081 | >16000 | 36 | | |
| 082 | >16000, >50000 | 33 | | |
| 083 | !66000 | 35 | | |
| 084 | 213 | 57 | | |
| 085 | 279 | 59 | | |
| 086 | 138 | 72 | | |
| 087 | 196 | 76 | | |
| 088 | 190 | 75 | | |
| 089 | 145 | 66 | | |
| 090 | 442 | 71 | | |
| 091 | 53 | 58 | | |
| 092 | 3445 | 57 | | |
| 093 | 216 | 68 | | |
| 094 | 200 | 74 | | |
| 095 | 99 | 63 | | |
| 096 | 27 | 59 | | |
| 097 | 123 | 78 | | |
| 098 | 25 | 57 | 387 | 69 |
| 099 | 22 | 55 | 808 | 67 |
| 100 | 23 | 74 | 120 | 43 |
| 101 | 59 | 69 | | |
| 102 | 84 | 61 | | |
| 103 | 141 | 70 | | |
| 104 | 22 | 60 | 604 | 59 |
| 105 | 203 | 75 | | |
| 106 | 524 | 42 | | |
| 107 | 901 | 64 | | |
| 108 | 175 | 60 | | |
| 109 | 825 | 56 | | |
| 110 | 159 | 37 | | |
| 111 | 273 | 48 | | |
| 112 | 289 | 60 | | |
| 113 | 239 | 73 | | |
| 114 | 305 | 64 | | |
| 115 | 1425 | 54 | | |
| 116 | 1280 | 72 | | |
| 117 | 468 | 61 | | |
| 118 | 1890 | 61 | | |
| 119 | 164 | 64 | | |
| 120 | 194 | 78 | | |
| 121 | 218 | 62 | | |
| 122 | 105 | 54 | | |
| 123 | 369 | 55 | | |
| 124 | 213 | 60 | | |
| 125 | 506 | 97 | | |
| 126 | 467 | 81 | | |
| 127 | 1050 | 66 | | |
| 128 | 380 | 60 | | |
| 129 | 1300 | 69 | | |
| 130 | 1050 | 70 | | |
| 131 | 2400 | 86 | | |
| 132 | 640 | 102 | | |
| 133 | 4100 | 92 | | |
| 134 | 1350 | 77 | | |
| 135 | 870 | 45 | | |
| 136 | 102 | 83 | | |
| 137 | 33 | 98 | 1750 | 66 |
| 138 | 27 | 56 | 9390 | 41 |
| 139 | 43 | 63 | 612 | 52 |
| 140 | 44 | 67 | | |
| 141 | 46 | 69 | 1130 | 66 |
| 142 | 60 | 70 | | |
| 143 | 39 | 71 | | |
| 144 | 129 | 76 | | |
| 145 | 194 | 72 | | |
| 146 | 314 | 71 | >50000 | 7 |
| 147 | 1273 | 69 | | |
| 148A | 143 | 68 | | |
| 148B | 519 | 64 | | |
| 149 | 118 | 77 | | |
| 150 | 71 | 79 | | |
| 151 | 69 | 97 | | |
| 152 | 431 | 89 | >50000 | 35 |
| 153 | 327 | 93 | | |
| 154 | 252 | 77 | | |
| 155 | 101 | 65 | | |
| 156 | 47 | 58 | >16000, >50000, | 25 |
| | | | >50000, >50000 | |
| 157 | 131 | 55 | | |
| 158 | 997 | 75 | | |
| 159 | 1179 | 91 | | |
| 160 | 40 | 67 | 1030 | 62 |
| 161 | 832 | 74 | | |
| 162 | 335 | 66 | | |
| 163 | 158 | 61 | 3500 | 65 |
| 164 | 71 | 49 | | |
| 165 | 97 | 43 | | |
| 166 | 269 | 47 | | |
| 167 | 103 | 80 | | |
| 168 | 181 | 85 | | |
| 169 | 1029 | 75 | | |
| 170 | >16000 | 36 | | |
| 171 | 4910 | 65 | | |
| 172 | 3238 | 63 | | |
| 173 | 320 | 92 | | |
| 174 | 5900 | 64 | | |
| 175 | 15000 | 67 | | |
| 176 | 880 | 53 | | |
| 177 | 970 | 69 | | |
| 178 | 490 | 56 | | |
| 179 | 176 | 65 | | |
| 180 | 902 | 63 | | |
| 181 | 2244 | 63 | | |
| 182 | 42 | 85 | | |
| 183 | 108 | 61 | | |
| 184 | 177 | 53 | | |
| 185 | 567 | 77 | >16000 | 22 |
| 186 | 462 | 97 | | |
| 187 | 29 | 64 | 199 | 41 |
| 188 | 2559 | 71 | | |
| 189 | 429 | 54 | | |
| 190 | 171 | 65 | | |
| 191 | 149 | 72 | | |
| 192 | 105 | 68 | | |
| 193 | 1065 | 79 | | |
| 194 | 208 | 65 | | |
| 195 | 245 | 64 | | |
| 196 | 102 | 71 | | |
| 197 | 4124 | 68 | | |
| 198 | 4740 | 60 | | |
| 199 | 1965 | 70 | | |
| 200 | 106 | 83 | | |
| 201 | 3304 | 73 | | |
| 202 | 278 | 83 | 947 | 60 |
| 203 | 1349 | 69 | | |
| 204 | 4036 | 60 | | |
| 205 | 396 | 74 | | |
| 206 | 10757 | 88 | | |
| 207 | 100 | 77 | 16000 | 20 |
| 208 | 552 | 67 | | |
| 209 | 742 | 64 | | |
| 210 | 491 | 71 | | |
| 211 | 404 | 102 | | |
| 212 | 406 | 85 | | |
| 213 | 818 | 61 | | |
| 214 | 94 | 55 | | |
| 215 | 1510 | 68 | | |
| 216 | 3350 | 45 | | |
| 217 | 4035 | 67 | | |
| 218 | 248 | 80 | | |
| 219 | >16000, >50000 | 23 | | |
| 220 | 3810 | 77 | | |
| 221 | >50000 | 30 | | |
| 222 | 8540, | 70 | | |
| | 8060, >16000, >16000 | | | |
| 223 | 1847 | 82 | | |
| 224 | 623 | 61 | | |
| 225 | 192 | 98 | | |
| 226 | 491 | 75 | | |
| 227 | 1771 | 69 | | |
| 228 | 1588 | 58 | | |
| 229 | 78 | 56 | | |
| 230 | 341, >50000 | 50 | | |
| 231 | 121 | 46 | | |
| 232 | 149 | 49 | 3330 | 61 |
| 233 | 305 | 79 | | |
| 234 | 695 | 43 | | |
| 235 | 787 | 69 | | |
| 236 | 232 | 77 | | |
| 237 | 235 | 59 | | |
| 238 | >16000 | | | |
| 239 | 152 | 99 | | |
| 240 | 3040 | 86 | | |
| 241 | >16000 | 34 | | |
| 242 | 2870, >16000 | 71 | | |
| 243 | 2720 | 70 | | |
| 244 | 115 | 80 | 654 | 51 |
| 245 | 5972 | 68 | | |
| 246 | 949 | 94 | | |
| 247 | 1108 | 59 | | |
| 248 | 1280 | 62 | | |
| 249 | 544 | 73 | | |
| 250 | 439 | 82 | | |
| 251 | 392 | 100 | | |
| 252 | 6465 | 93 | | |
| 253 | 236 | 71 | | |
| 254 | 302 | 71 | | |
| 255 | 271 | 64 | | |
| 258 | | | 260 | 52 |
| 264 | 460 | 80 | | |
| 269 | >50000 | 14 | | |
| 270 | | | 1240 | 44 |
| 271 | | | >16000 | 31 |
| 272 | | | >16000 | 25 |
| 273 | | | 149 | 45 |
| 274 | 2400 | 64 | | |
| 275 | >50000 | 35 | 805 | 44 |
| 276 | >50000 | 35 | | |
| 277 | 185 | 45 | | |
| 278 | 180 | 65 | | |
| 279 | 540 | 85 | | |
| 280 | 29 | 55 | | |
| 281 | 140 | 69 | | |

### In Vitro Mechanistic Cellular Assay

Human whole blood assay (human WBA) was prepared from the blood of healthy female volunteers. In this assay, the efficacy of P2X4 antagonists on the production of IL-13 in whole blood after ATP stimulation (3 mM for 1 h) following priming of the cells with lipopolysacaride (LPS, 100 ng/ml for 2 h) was evaluated. After incubation, supernatant was taken following centrifugation and the formation of IL-13 was assayed using standard ELISA kits. IC₅₀ values were calculated using GraphPad PRISM program with nonlinear regression curve fit. Data are presented as total concentration.

| | human WBA P2X4 |
|---|---|
| Example Number | avg IC₅₀ total [μM] |
| 015 | 2.54 |
| 164 | 10.7 |
| 187 | 4.00 |
| 229 | 30.0 |
| 244 | 21.9 |

### In Vitro Electrophysiological Assay

### Cell Culture Conditions

HEK-293 mito-Photina pcDNA3(neo-)/pPURO N/pcDNA3_P2RX4, clone 2a/4 (HEK-293 mito-Photina/hP2RX4) cells were cultured in EMEM Minimum Essential Medium Eagle with Earl's salts Balanced Salt Solution (BioWhittaker cat. BE12-125F) supplemented with 5 mL of 200 mM Ultraglutamine1 (BioWhittaker cat. BE17-605E/U1), 5 mL of 100× Penicillin/Streptomycin (BioWhittaker cat. DE17-602E, final concentration 1%), 4 mL of 50 mg/mL G418 (Sigma cat. G8168-100 mL; final concentration 400 μg/mL), 10 μL of 10 mg/mL Puromicin (InvivoGen cat. ant-pr-1; final concentration 0.2 μg/mL) and 50 mL of Fetal Bovine Serum (Sigma cat. F7524; final concentration 10%).

### Experimental Protocol

HEK-293 cell lines are seeded 72 or 96 hours before experiment, at a concentration of 5 or 2.5 million cells, respectively onto a T225 flask. Just before the experiments cells are washed twice with D-PBS w/o Ca2+/Mg2+(Euroclone cat. ECB4004L) and detached from the flask with trypsin-EDTA (Sigma, cat. T4174 diluted 1/10). Cells are then re-suspended in the suspension solution: 25 mL EX-CELL ACF CHO medium (Sigma, cat. C5467); 0.625 mL HEPES (BioWhittaker, cat. BE17-737E); 0.25 mL of 100×

Penicillin/Streptomycin (BioWhittaker, cat. DE17-602E), 0.1 mL of Soybean Trypsin Inhibitor 10 mg/mL (Sigma, cat. T6522) and placed on the QPatch 16X.

### Compound Preparation and Storage

Compound stock solutions (10 mM; 100% DMSO; stored at −20° C.) were used. Fresh solutions from stock were prepared just before the experiments (0.1% final DMSO concentration).

DMSO solution was obtained from SIGMA (cat.# D-5879) and stored at room temperature.

Patch Clamp Analysis with QPatch16X (FIG. 1)

Standard whole-cell voltage clamp experiments are performed at room temperature using the multihole technology.

For the voltage clamp experiments on hP2X4 receptor, data are sampled at 2 KHz. After establishment of the seal and the passage in the whole cell configuration, the cells are held at −90 mV and the hP2X4 receptor current is evoked by the agonist in the absence (vehicle period, i.e. 0.1% DMSO) or in the presence of the compound under investigation at increasing concentrations; see the application protocol in FIG. 1 in which the intracellular solution contained (mM) 135 CsF, 10 NaCl, 1 EGTA, 10 HEPES (pH 7.2 with CsOH) and whereas the extracellular solution (mM) 145 NaCl, 4 KCl, 0.5 MgCl2, 1 CaCl2, 10 HEPES, 10 Glucose (pH 7.4 with NaOH).

Output: the maximum inward current induced by the agonist: ATP 5 microM.

For data collection, the Sophion software was used and the analysis was performed off-line using Excel and GraphPad Prism.

When possible, i.e. when the % of inhibition with the highest concentration tested was more than 50%, the dose-response curves data were fitted with the following equation:

Y=100/(1+10^{∧}((Log IC50−X)*HillSlope))

X: log of concentration

Y: normalized response, 100% down to 0%, decreasing as X increases.

Log IC50: same log units as X

HillSlope: slope factor or hill slope, unitless

| | human P2X4 | | |
|---|---|---|---|
| | electrophysiology | | |
| Example Number | IC₅₀ [nM] | std. error logIC50 | n |
| 163 | 261 | 0.08653 | 5 |
| 244 | 274 | 0.09535 | 5 |

### In Vivo Studies

Efficacy in CFA Inflammation Model with Pain Read Out

Wild type female c57bl/6 mice (Taconic) received intraplantar injection of complete Freund's adjuvant (CFA) (30 μl, 1 mg/ml, Sigma) into the left hind paw under isoflurane anesthesia. Animals were administered oral dosing of Example 244 (50 mg/kg, n=10/group) on day 1 and on day 2 post-CFA injection. Spontaneous pain-related behavior in freely moving animals was assessed using the automated dynamic weight bearing device (DWB, Bioseb, France) according to published and validated protocols (Robinson et al., 2012; Tetreault et al., 2011; Gruen et al. 2014). For behavioral testing, the animal was placed inside a Plexiglas chamber and allowed to move freely within the apparatus for a 5 min period, subsequently pain behavior is recorded for a test period of another 5 min. Example 244 significantly reduced pain behavior as assessed by DWB after CFA-induced inflammation (see FIG. 2).

Statistical analysis is performed with one-way analysis of variance, followed by Bonferroni's multiple comparison test vs. vehicle controls using the GraphPad PRISM software, *p<0.05, **p<0.01.

In conclusion, Example 244 significantly reduced spontaneous pain behavior in mice at 24-48 h after CFA-induced inflammation as can be seen in FIG. 2.

Effect on PGE2 Levels after CFA-Induced Inflammation in Mice

Female c57bl/6 mice (Taconic) received intraplantar injection of complete Freund's adjuvant (CFA) (30 μl, 1 mg/ml, Sigma) into the left hind paw under isoflurane anesthesia. Animals received single oral dosing of Example 244 (12.5, 25, 50 mg/kg). At termination of the experiments (48 h after CFA, 1 h after dose), hind paw tissue is collected for analysis of PGE2 levels (ELISA, Cayman Chemical item no. 514531). The data demonstrate that both compounds tested significantly inhibited CFA-induced PGE2 formation in the ipsilateral inflamed hind paw. FIG. 3 demonstrates strong efficacy of Example 244 on the inhibition of PGE2 release in inflamed paws of mice after CFA injection. The compound dose-dependently reduced PGE2 levels evoked by CFA inflammation. In addition, PGE2 levels after treatment with Example 244 were still in the range of PGE2 levels of PBS treated animals, indicating that residual PGE2 levels are maintained after P2X4 antagonism.

Statistical analysis is performed with one-way analysis of variance, followed by Bonferroni's multiple comparison test vs. vehicle controls using the GraphPad PRISM software, *p<0.05, **p<0.01.

### FIGURES

FIG. 1 represents the application protocol for the whole-cell voltage clamp experiments in which the intracellular solution contained (mM) 135 CsF, 10 NaCl, 1 EGTA, 10 HEPES (pH 7.2 with CsOH) and whereas the extracellular solution (mM) 145 NaCl, 4 KCl, 0.5 MgCl2, 1 CaCl₂), 10 HEPES, 10 Glucose (pH 7.4 with NaOH)

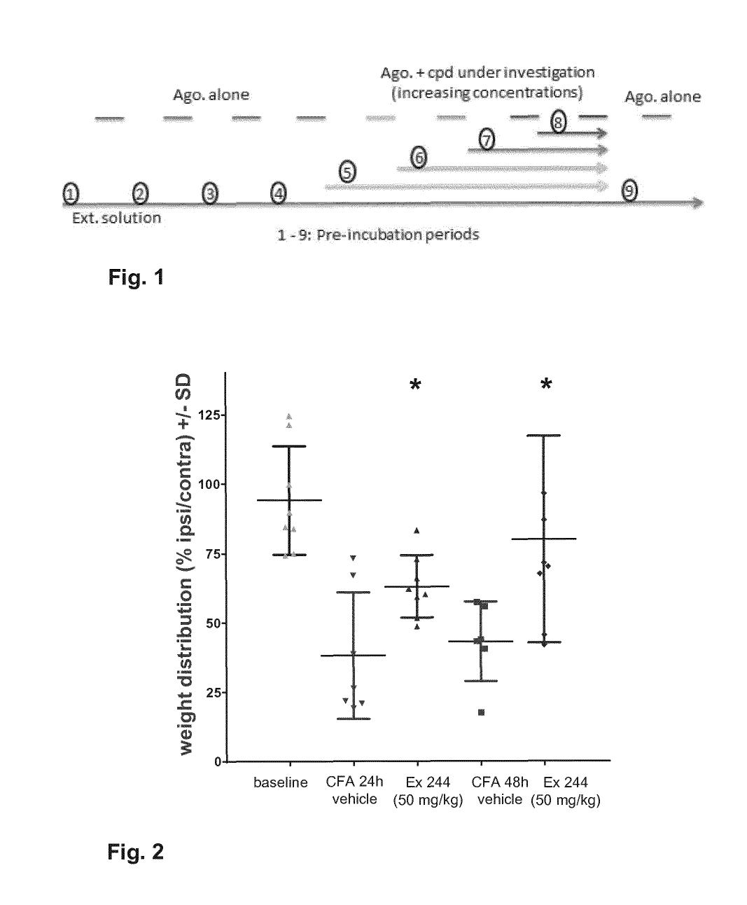

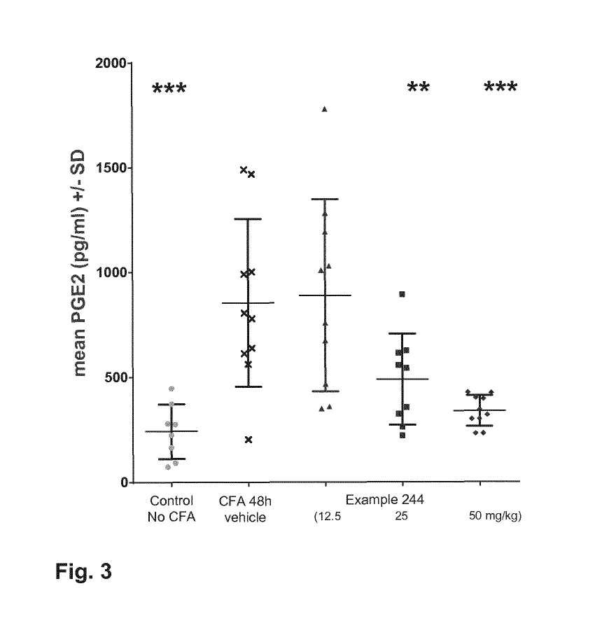

As nonbinding explanatory example of compounds according to the invention FIG. 2 represents the effect of Example 244 on CFA-induced pain behavior in mice; as already reported above Example 244 significantly reduced spontaneous pain behavior in mice at 24-48 h after CFA-induced inflammation.

Furthermore, in FIG. 3 the effect of Example 244 on PGE2 levels in mice after CFA-induced inflammation is shown and again the data demonstrate strong efficacy of Example 244 on the inhibition of PGE2 release in inflamed paws of mice after CFA injection.

1. A compound of formula (I) 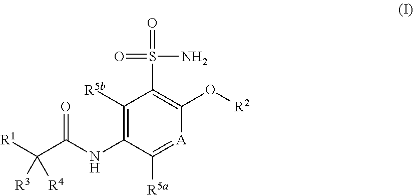
in which:
A represents CR⁵ or N;
R¹ represents a group selected from: 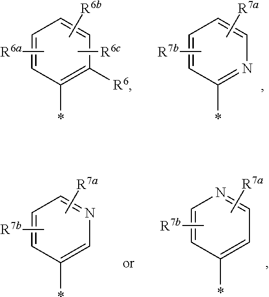
wherein * indicates the point of attachment of said group with the rest of the molecule;
R² represents C₃-C₆-cycloalkyl, C₃-C₆-cycloalkyl-C₁-C₄-alkyl, 4- to 6-membered heterocycloalkyl, 4- to 6-membered heterocycloalkyl-C₁-C₄-alkyl, phenyl, phenyl-C₁-C₄-alkyl, heteroaryl or heteroaryl-C₁-C₄-alkyl,
wherein said groups are optionally substituted one to four times with R¹¹, being, independently from each other, the same or different, or
substituted one time with R^{11a} and optionally one to two times with R¹¹ being independently from each other, the same or different, or
substituted with two adjacent substituents R¹¹ which together represent a methylendioxy group to form a 5-membered ring or
substituted with one to five deuterium atoms and optionally one to two times with R¹¹ being, independently from each other, the same or different, or
R² represents branched (C₁-C₄-alkyl)-C₁-C₄-alkyl;
R³ represents hydrogen, deuterium, fluoro or methyl;
R⁴ represents hydrogen, deuterium or fluoro;
R⁵, R^{5a} and R^{5b} are the same or different and represent, independently from each other, hydrogen, halogen, C₁-C₄-alkyl, C₁-C₄-haloalkyl, C₁-C₄-alkoxy or C₁-C₄-haloalkoxy;
R⁶, R^{6a}, R^{6b} and R^{6c} are the same or different and represent, independently from each other, respectively
R⁶ hydrogen, halogen, cyano, C₁-C₄-alkyl, C₁-C₄-haloalkyl, C₁-C₄-alkoxy, C₁-C₄-haloalkoxy, HO—(C₂-C₄-alkoxy)-, (C₁-C₄-alkoxy)-(C₂-C₄-alkoxy)- or F₃C—S—;
R^{6a} hydrogen, halogen, hydroxy, nitro, cyano, C₁-C₄-alkyl, C₃-C₆-cycloalkyl, C₁-C₄-haloalkyl, C₁-C₄-alkoxy, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—;
R^{6b} hydrogen, halogen, hydroxy, nitro, cyano, C₁-C₄-alkyl, C₃-C₆-cycloalkyl, C₁-C₄-haloalkyl, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—; or
R^{6a} and R^{6b} adjacent to each other together represent a group selected from —O—CH₂—CH₂—, —O—CH₂—O— or —O—CH₂—CH₂—O—;
R^{6c} hydrogen or halogen;
R^{7a} and R^{7b} are the same or different and represent, independently from each other, hydrogen, hydroxy, halogen, C₁-C₄-alkyl or C₁-C₄-haloalkyl;
R⁸ represents, independently from each respective occurrence, C₁-C₆-alkyl, C₁-C₄-alkoxy-C₁-C₄-alkyl, C₃-C₆-cycloalkyl or C₁-C₄-haloalkyl;
R⁹ and R¹⁰ are the same or different and represent, independently from each other, hydrogen, C₁-C₄-alkyl, C₃-C₆-cycloalkyl, C₁-C₄-haloalkyl, (C₁-C₄-alkoxy)-(C₂-C₄-alkyl), phenyl or heteroaryl, wherein said phenyl and heteroaryl groups are optionally substituted one to three times, independently from each other, with hydrogen, halogen, C₁-C₄-alkyl, C₁-C₄-haloalkyl, C₁-C₄-alkoxy or C₁-C₄-haloalkoxy,
R^{9a} and R^{10a} together with the nitrogen atom to which they are attached form a 4- to 6-membered nitrogen containing heterocyclic ring, said ring optionally containing one additional heteroatom selected from O, NH, NR^{a} in which R^{a} represents a C₁-C₆-alkyl- or C₁-C₆-haloalkyl- group, or S and being optionally substituted, one to three times, independently from each other, with halogen or C₁-C₄-alkyl;
R¹¹ represents, independently from each other, halogen, hydroxy, nitro, cyano, C₁-C₄-alkyl, C₂-C₄-alkenyl, C₁-C₄-haloalkyl, C₁-C₄-hydroxyalkyl, C₁-C₄-alkoxy, C₁-C₄-haloalkoxy, HO—(C₂-C₄-alkoxy)-, (C₁-C₄-alkoxy)-(C₂-C₄-alkoxy)-, (C₁-C₄-haloalkyl)-S—, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—;
R^{11a} represents a group selected from C₃-C₆-cycloalkyl, morpholino, R^{9a}R^{10a}N—; R^{9a}R^{10a}N—C(O)—; a 5- to 6-membered heteroaryl, which is optionally substituted with methyl, or
represents: 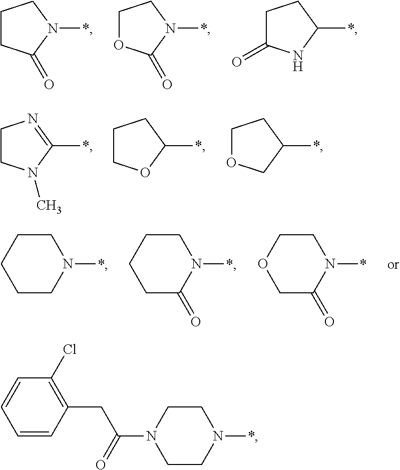
wherein * indicates the point of attachment of said group with the rest of the molecule; or
an N-oxide, a salt, a hydrate, a solvate, a tautomer or a stereoisomer of said compound, or a salt of said N-oxide, tautomer or stereoisomer.

2. A compound of formula (Ia) according to claim 1 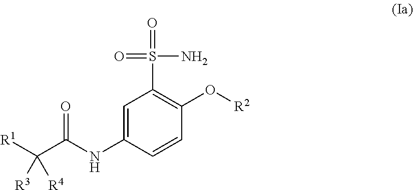
wherein
R¹ represents a group selected from: 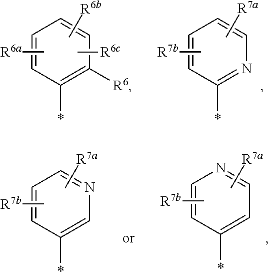
wherein * indicates the point of attachment of said group with the rest of the molecule;
R² represents C₃-C₆-cycloalkyl, C₃-C₆-cycloalkyl-C₁-C₄-alkyl, 4- to 6-membered heterocycloalkyl, 4- to 6-membered heterocycloalkyl-C₁-C₄-alkyl, phenyl, phenyl-C₁-C₄-alkyl, heteroaryl or heteroaryl-C₁-C₄-alkyl,
wherein said groups are optionally substituted one to four times with R¹¹, being, independently from each other, the same or different, or
substituted one time with R^{11a} and optionally one to two times with R¹¹ being independently from each other, the same or different, or
substituted with two adjacent substituents R¹¹ which together represent a methylendioxy group to form a 5-membered ring or
substituted with one to five deuterium atoms and optionally one to two times with R¹¹ or R^{11a} being, independently from each other, the same or different; or
R² represents branched (C₁-C₄-alkyl)-C₁-C₄-alkyl;
R³ represents hydrogen, deuterium, fluoro or methyl;
R⁴ represents hydrogen, deuterium or fluoro;
R⁶, R^{6a}, R^{6b} and R^{6c} are the same or different and represent, independently from each other, respectively
R⁶ hydrogen, halogen, cyano, C₁-C₄-alkyl, C₁-C₄-haloalkyl, C₁-C₄-alkoxy, C₁-C₄-haloalkoxy, HO—(C₂-C₄-alkoxy)-, (C₁-C₄-alkoxy)-(C₂-C₄-alkoxy)- or F₃C—S—;
R^{6a} hydrogen, halogen, hydroxy, nitro, cyano, C₁-C₄-alkyl, C₃-C₆-cycloalkyl, C₁-C₄-haloalkyl, C₁-C₄-alkoxy, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—;
R^{6b} hydrogen, halogen, hydroxy, nitro, cyano, C₁-C₄-alkyl, C₃-C₆-cycloalkyl, C₁-C₄-haloalkyl, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—; or
R^{6a} and R^{6b} adjacent to each other together represent a group selected from —O—CH₂—CH₂—, —O—CH₂—O— or —O—CH₂—CH₂—O—;
R^{6c} hydrogen or halogen;
R^{7a} and R^{7b} are the same or different and represent, independently from each other, hydrogen, hydroxy, halogen, C₁-C₄-alkyl or C₁-C₄-haloalkyl;
R⁸ represents, independently from each respective occurrence, C₁-C₆-alkyl, C₁-C₄-alkoxy-C₁-C₄-alkyl, C₃-C₆-cycloalkyl or C₁-C₄-haloalkyl;
R⁹ and R¹⁰ are the same or different and represent, independently from each other, hydrogen, C₁-C₄-alkyl, C₃-C₆-cycloalkyl, C₁-C₄-haloalkyl, (C₁-C₄-alkoxy)-(C₂-C₄-alkyl), phenyl or heteroaryl, wherein said phenyl and heteroaryl groups are optionally substituted one to three times, independently from each other, with hydrogen, halogen, C₁-C₄-alkyl, C₁-C₄-haloalkyl, C₁-C₄-alkoxy or C₁-C₄-haloalkoxy;
R^{9a} and R^{10a} together with the nitrogen atom to which they are attached form a 4- to 6-membered nitrogen containing heterocyclic ring, said ring optionally containing one additional heteroatom selected from O, NH, NR^{a} in which R^{a} represents a C₁-C₆-alkyl- or C₁-C₆-haloalkyl- group, or S and being optionally substituted, one to three times, independently from each other, with halogen or C₁-C₄-alkyl;
R¹¹ represents, independently from each other, halogen, hydroxy, nitro, cyano, C₁-C₄-alkyl, C₂-C₄-alkenyl, C₁-C₄-haloalkyl, C₁-C₄-hydroxyalkyl, C₁-C₄-alkoxy, C₁-C₄-haloalkoxy, HO—(C₂-C₄-alkoxy)-, (C₁-C₄-alkoxy)-(C₂-C₄-alkoxy)-, (C₁-C₄-haloalkyl)-S—, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—;
R^{11a} represents a group selected from C₃-C₆-cycloalkyl, morpholino, R^{9a}R^{10a}N—; R^{9a}R^{10a}N—C(O)—; a 5- to 6-membered heteroaryl, which is optionally substituted with methyl or
represents: 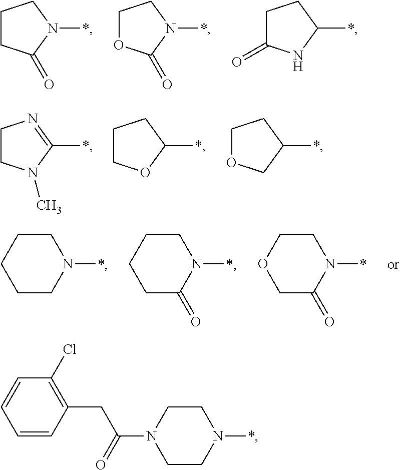
wherein * indicates the point of attachment of said group with the rest of the molecule; or
an N-oxide, a salt, a hydrate, a solvate, a tautomer or a stereoisomer of said compound, or a salt of said N-oxide, tautomer or stereoisomer.

3. A compound of formula (Ib) according to claim 1 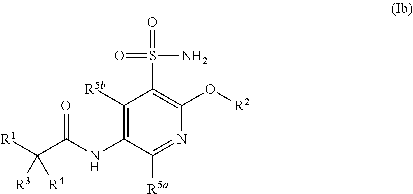
wherein
R¹ represents a group selected from: 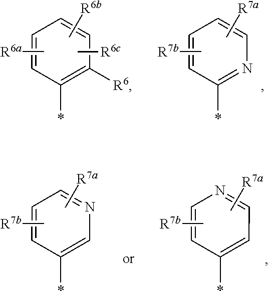
wherein * indicates the point of attachment of said group with the rest of the molecule;
R² represents C₃-C₆-cycloalkyl, C₃-C₆-cycloalkyl-C₁-C₄-alkyl, 4- to 6-membered heterocycloalkyl, 4- to 6-membered heterocycloalkyl-C₁-C₄-alkyl, phenyl, phenyl-C₁-C₄-alkyl, heteroaryl or heteroaryl-C₁-C₄-alkyl,
wherein said groups are optionally substituted one to four times with R¹¹, being, independently from each other, the same or different, or
substituted one time with R^{11a} and optionally one to two times with R¹¹ being independently from each other, the same or different, or
substituted with two adjacent substituents R¹¹ which together represent a methylendioxy group to form a 5-membered ring or
substituted with one to five deuterium atoms and optionally one to two times with R¹¹ being, independently from each other, the same or different; or
R² represents branched (C₁-C₄-alkyl)-C₁-C₄-alkyl;
R³ represents hydrogen, fluoro or methyl;
R⁴ represents hydrogen or fluoro;
R^{5a} and R^{5b} are the same or different and represent, independently from each other, hydrogen, halogen, C₁-C₄-alkyl, C₁-C₄-haloalkyl, C₁-C₄-alkoxy or C₁-C₄-haloalkoxy;
R⁶, R^{6a}, R^{6b} and R^{6c} are the same or different and represent, independently from each other, respectively
R⁶ hydrogen, halogen, cyano, C₁-C₄-alkyl, C₁-C₄-haloalkyl, C₁-C₄-alkoxy, C₁-C₄-haloalkoxy, HO—(C₂-C₄-alkoxy)-, (C₁-C₄-alkoxy)-(C₂-C₄-alkoxy)- or F₃C—S—;
R^{6a} hydrogen, halogen, hydroxy, nitro, cyano, C₁-C₄-alkyl, C₃-C₆-cycloalkyl, C₁-C₄-haloalkyl, C₁-C₄-alkoxy, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—;
R^{6b} hydrogen, halogen, hydroxy, nitro, cyano, C₁-C₄-alkyl, C₃-C₆-cycloalkyl, C₁-C₄-haloalkyl, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—; or
R^{6a} and R^{6b} adjacent to each other together represent a group selected from —O—CH₂—CH₂—, —O—CH₂—O— or —O—CH₂—CH₂—O—;
R^{6c} hydrogen or halogen;
R^{7a} and R^{7b} are the same or different and represent, independently from each other, hydrogen, hydroxy, halogen, C₁-C₄-alkyl or C₁-C₄-haloalkyl;
R⁸ represents, independently from each respective occurrence, C₁-C₆-alkyl, C₁-C₄-alkoxy-C₁-C₄-alkyl, C₃-C₆-cycloalkyl or C₁-C₄-haloalkyl;
R⁹ and R¹⁰ are the same or different and represent, independently from each other, hydrogen, C₁-C₄-alkyl, C₃-C₆-cycloalkyl, C₁-C₄-haloalkyl, (C₁-C₄-alkoxy)-(C₂-C₄-alkyl), phenyl or heteroaryl, wherein said phenyl and heteroaryl groups are optionally substituted one to three times, independently from each other, with hydrogen, halogen, C₁-C₄-alkyl, C₁-C₄-haloalkyl, C₁-C₄-alkoxy or C₁-C₄-haloalkoxy;
R^{9a} and R^{10a} together with the nitrogen atom to which they are attached form a 4- to 6-membered nitrogen containing heterocyclic ring, said ring optionally containing one additional heteroatom selected from O, NH, NR^{a} in which R^{a} represents a C₁-C₆-alkyl- or C₁-C₆-haloalkyl- group, or S and being optionally substituted, one to three times, independently from each other, with halogen or C₁-C₄-alkyl;
R¹¹ represents, independently from each other, halogen, hydroxy, nitro, cyano, C₁-C₄-alkyl, C₂-C₄-alkenyl, C₁-C₄-haloalkyl, C₁-C₄-hydroxyalkyl, C₁-C₄-alkoxy, C₁-C₄-haloalkoxy, HO—(C₂-C₄-alkoxy)-, (C₁-C₄-alkoxy)-(C₂-C₄-alkoxy)-, (C₁-C₄-haloalkyl)-S—, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—;
R^{11a} represents a group selected from C₃-C₆-cycloalkyl, morpholino, R^{9a}R^{10a}N—; R^{9a}R^{10a}N—C(O)—; a 5- to 6-membered heteroaryl, which is optionally substituted with methyl or
represents: 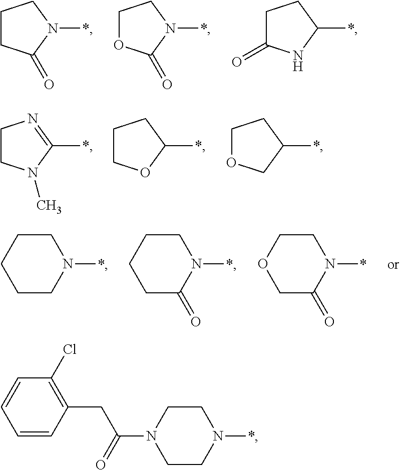
wherein * indicates the point of attachment of said group with the rest of the molecule; or
an N-oxide, a salt, a hydrate, a solvate, a tautomer or a stereoisomer of said compound, or a salt of said N-oxide, tautomer or stereoisomer.

4. A compound of formula (Ia) according to claim 2 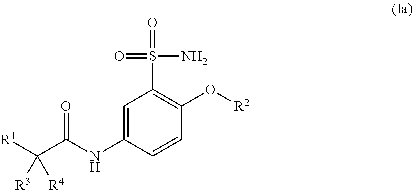
wherein
R¹ represents a group selected from: 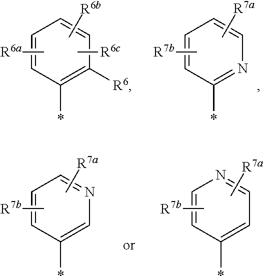
wherein * indicates the point of attachment of said group with the rest of the molecule;
R² represents C₃-C₆-cycloalkyl, C₃-C₆-cycloalkyl-C₁-C₄-alkyl, 4- to 6-membered heterocycloalkyl, 4- to 6-membered heterocycloalkyl-C₁-C₄-alkyl, phenyl, phenyl-C₁-C₄-alkyl, heteroaryl or heteroaryl-C₁-C₄-alkyl,
wherein said groups are optionally substituted one to four times with R¹¹, being, independently from each other, the same or different, or
substituted one time with R^{11a} and optionally one to two times with R¹¹ being independently from each other, the same or different, or
substituted with two adjacent substituents R¹¹ which together represent a methylendioxy group to form a 5-membered ring or
substituted with one to five deuterium atoms and optionally one to two times with R¹¹ being, independently from each other, the same or different;
R³ represents hydrogen, fluoro or methyl;
R⁴ represents hydrogen or fluoro;
R⁶, R^{6a}, R^{6b} and R^{6c} are the same or different and represent, independently from each other, respectively
R⁶ hydrogen, fluoro, chloro, bromo, cyano, C₁-C₄-alkyl, difluoromethyl, trifluoromethyl, methoxy, ethoxy, difluoromethoxy, trifluoromethoxy, 2-hydroxy-ethoxy, 2-methoxy-ethoxy or F₃C—S—;
R^{6a} hydrogen, fluoro, chloro, bromo, hydroxy, cyano, methyl, difluoromethyl, trifluoromethyl, methoxy, 2-hydroxy-ethoxy, 2-methoxy-ethoxy or R⁹R¹⁰N—C(O)—;
R^{6b} hydrogen, fluoro, chloro or bromo; or
R^{6a} and R^{6b} adjacent to each other together represent a group selected from —O—CH₂—CH₂— or —O—CH₂—CH₂—O—;
R^{6c} hydrogen or halogen;
R^{7a} and R^{7b} are the same or different and represent, independently from each other, hydrogen, chloro, methyl, difluoromethyl or trifluoromethyl;
R⁸ represents methyl;
R⁹ and R¹⁰ are the same or different and represent, independently from each other, hydrogen, methyl, cyclopropyl or 2-methoxy-ethyl, or
R^{9a} and R^{10a} together with the nitrogen atom to which they are attached form a 4- to 6-membered nitrogen containing heterocyclic ring, said ring optionally containing one additional heteroatom selected from O, NH, NCH₃ or S and being optionally substituted, one to three times, independently from each other, with halogen or methyl
R¹¹ represents, independently from each other, halogen, hydroxy, nitro, cyano, C₁-C₄-alkyl, C₂-C₄-alkenyl, C₁-C₄-haloalkyl, C₁-C₄-hydroxyalkyl, C₁-C₄-alkoxy, C₁-C₄-haloalkoxy, HO—(C₂-C₄-alkoxy)-, (C₁-C₄-alkoxy)-(C₂-C₄-alkoxy)-, (C₁-C₄-haloalkyl)-S—, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—;
R^{11a} represents a group selected from C₃-C₆-cycloalkyl, morpholino, R^{9a}R^{10a}N—; R^{9a}R^{10a}N—C(O)—; a 5- to 6-membered heteroaryl, which is optionally substituted with methyl or
represents a group selected from: 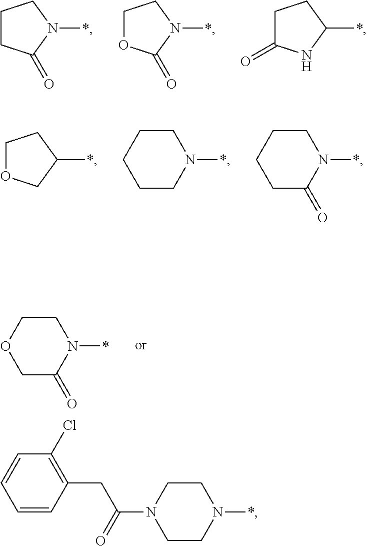
wherein * indicates the point of attachment of said group with the rest of the molecule; or
an N-oxide, a salt, a hydrate, a solvate, a tautomer or a stereoisomer of said compound, or a salt of said N-oxide, tautomer or stereoisomer.

5. A compound of formula (Ia) according to claim 2 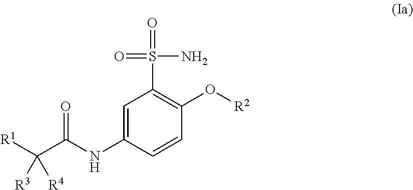
wherein
R¹ represents a group selected from: 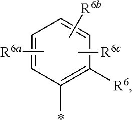
wherein * indicates the point of attachment of said group with the rest of the molecule;
R² represents C₄-C₆-cycloalkyl, C₃-C₆-cycloalkyl-methyl, 4- to 6-membered heterocycloalkyl, 4- to 6-membered heterocycloalkyl-methyl, phenyl, phenyl-C₁-C₂-alkyl, heteroaryl, heteroaryl-methyl wherein said groups are optionally substituted one to four times with R¹¹, being, independently from each other, the same or different, or
substituted one time with R^{11a} and optionally one to two times with R¹¹ being independently from each other, the same or different, or
substituted with two adjacent substituents R¹¹ which together represent a methylendioxy group to form a 5-membered ring;
R³ represents hydrogen or methyl;
R⁴ represents a hydrogen;
R⁶, R^{6a} and R^{6b} are the same or different and represent, independently from each other, respectively
R⁶ hydrogen, fluoro, chloro, bromo, cyano, C₁-C₄-alkyl, difluoromethyl, trifluoromethyl, methoxy, ethoxy, difluoromethoxy, trifluoromethoxy, 2-hydroxy-ethoxy, 2-methoxy-ethoxy or F₃C—S—;
R^{6a} hydrogen, fluoro, chloro, bromo, hydroxy, cyano, methyl, difluoromethyl, trifluoromethyl, methoxy, 2-hydroxy-ethoxy, 2-methoxy-ethoxy or R⁹R¹⁰N—C(O)—;
R^{6b} hydrogen, fluoro, chloro or bromo; or
R^{6a} and R^{6b} adjacent to each other together represent a group selected from —O—CH₂—CH₂— or —O—CH₂—CH₂—O—;
R^{6c} represents hydrogen or halogen;
R⁹ and R¹⁰ are the same or different and represent, independently from each other, hydrogen, methyl, cyclopropyl or 2-methoxy-ethyl, or
R^{9a} and R^{10a} together with the nitrogen atom to which they are attached form a 4- to 6-membered nitrogen containing heterocyclic ring, said ring optionally containing one additional heteroatom selected from O, NH, NR^{a} in which R^{a} represents a C₁-C₆-alkyl- or C₁-C₆-haloalkyl- group, or S and being optionally substituted, one to three times, independently from each other, with halogen or methyl;
R¹¹ represents, independently from each other, halogen, hydroxy, nitro, cyano, C₁-C₄-alkyl, C₂-C₄-alkenyl, C₁-C₄-haloalkyl, C₁-C₄-hydroxyalkyl, C₁-C₄-alkoxy, C₁-C₄-haloalkoxy, HO—(C₂-C₄-alkoxy)-, (C₁-C₄-alkoxy)-(C₂-C₄-alkoxy)-, (C₁-C₄-haloalkyl)-S—, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—;
R^{11a} represents a group selected from C₃-C₆-cycloalkyl, morpholino, R^{9a}R^{10a}N—; R^{9a}R^{10a}N—C(O)—; a 5- to 6-membered heteroaryl, which is optionally substituted with methyl or
represents a group selected from: 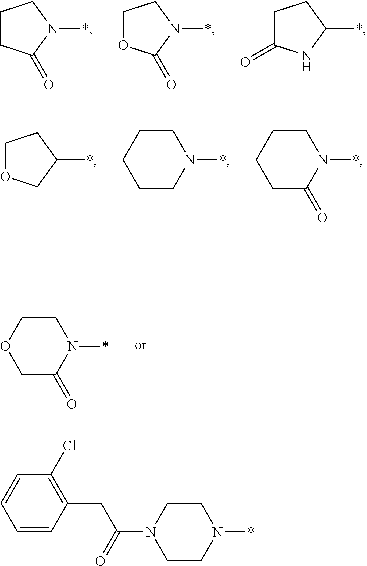
wherein * indicates the point of attachment of said group with the rest of the molecule; or
an N-oxide, a salt, a hydrate, a solvate, a tautomer or a stereoisomer of said compound, or a salt of said N-oxide, tautomer or stereoisomer.

6. A compound according to claim 1 wherein
R¹ represents a group selected from: 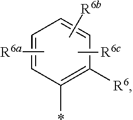
wherein * indicates the point of attachment of said group with the rest of the molecule;
R⁶, R^{6a} and R^{6b} are the same or different and represent, independently from each other, respectively
R⁶ halogen, cyano, C₁-C₄-alkyl, C₁-C₄-haloalkyl, C₁-C₄-alkoxy, C₁-C₄-haloalkoxy, HO—(C₂-C₄-alkoxy)-, (C₁-C₄-alkoxy)-(C₂-C₄-alkoxy)-;
R^{6a} hydrogen, halogen, hydroxy, nitro, cyano, C₁-C₄-alkyl, C₃-C₆-cycloalkyl, C₁-C₄-haloalkyl, C₁-C₄-alkoxy, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—;
R^{6b} hydrogen, halogen, hydroxy, nitro, cyano, C₁-C₄-alkyl, C₃-C₆-cycloalkyl, C₁-C₄-haloalkyl, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—;
R^{6c} represents hydrogen.

7. A compound according to claim 1 wherein
R¹ represents a group selected from: 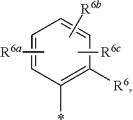
wherein * indicates the point of attachment of said group with the rest of the molecule; and
R⁶, R^{6a} and R^{6b} are the same or different and represent, independently from each other, respectively
R⁶ hydrogen, fluoro, chloro, bromo, cyano, C₁-C₄-alkyl, difluoromethyl, trifluoromethyl, methoxy, ethoxy, difluoromethoxy, trifluoromethoxy, 2-hydroxy-ethoxy, 2-methoxy-ethoxy or F₃C—S—;
R^{6a} hydrogen, fluoro, chloro, bromo, hydroxy, cyano, methyl, difluoromethyl, trifluoromethyl, methoxy, 2-hydroxy-ethoxy, 2-methoxy-ethoxy;
R^{6b} hydrogen, halogen, hydroxy, nitro, cyano, C₁-C₄-alkyl, C₃-C₆-cycloalkyl, C₁-C₄-haloalkyl, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—
R^{6c} represents hydrogen.

8. A compound according to claim 1 wherein
R¹ represents a group selected from: 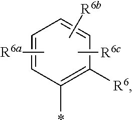
wherein * indicates the point of attachment of said group with the rest of the molecule; and
R⁶, R^{6a} and R^{6b} are the same or different and represent, independently from each other, respectively
R⁶ fluoro, chloro, bromo, cyano, C₁-C₄-alkyl, difluoromethyl, trifluoromethyl, methoxy, ethoxy, difluoromethoxy, trifluoromethoxy or F₃C—S—;
R^{6a} hydrogen, fluoro, chloro, bromo, hydroxy, cyano, methyl, difluoromethyl, trifluoromethyl, methoxy, 2-hydroxy-ethoxy, 2-methoxy-ethoxy;
R^{6b} hydrogen, halogen, hydroxy, nitro, cyano, C₁-C₄-alkyl, C₃-C₆-cycloalkyl, C₁-C₄-haloalkyl, C₁-C₄-haloalkoxy, HO—(C₂-C₄-alkoxy)-, (C₁-C₄-alkoxy)-(C₂-C₄-alkoxy)-, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—
R^{6c} represents hydrogen.

9. A compound according to claim 1 wherein
R¹ represents a group selected from: 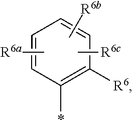
wherein * indicates the point of attachment of said group with the rest of the molecule; and
R⁶ represents hydrogen or halogen and
R^{6a} and R^{6b} adjacent to each other together represent a group selected from —O—CH₂—CH₂— or —O—CH₂—CH₂—O—
R^{6c} represents hydrogen.

10. A compound according to claim 1 wherein
R¹ represents a group selected from: 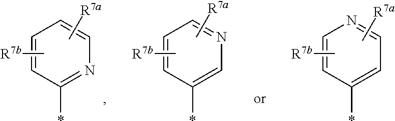
wherein * indicates the point of attachment of said group with the rest of the molecule;
R^{7a} and R^{7b} are the same or different and represent, independently from each other, hydrogen, fluoro, chloro, C₁-C₄-alkyl, difluoromethyl or trifluoromethyl.

11. A compound according to claim 1 wherein
R² represents a group selected from: 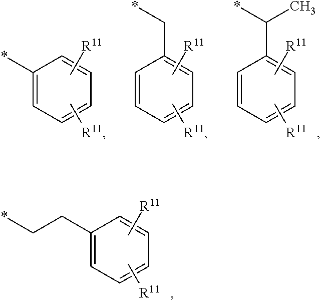
wherein * indicates the point of attachment of said group with the rest of the molecule and in which,
R¹¹ represents independently from each other, hydrogen, halogen, hydroxy, nitro, cyano, C₁-C₄-alkyl, C₂-C₄-alkenyl, C₁-C₄-haloalkyl, C₁-C₄-hydroxyalkyl, C₁-C₄-alkoxy, C₁-C₄-haloalkoxy, HO—(C₂-C₄-alkoxy)-, (C₁-C₄-alkoxy)-(C₂-C₄-alkoxy)-, (C₁-C₄-haloalkyl)-S—, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—.

12. A compound according to claim 1 wherein
R² represents a group selected from: 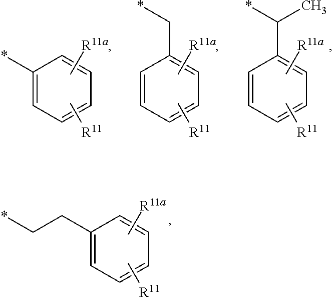
wherein * indicates the point of attachment of said group with the rest of the molecule and in which R¹¹ and R^{11a} are respectively
R¹¹ represents, hydrogen, halogen, hydroxy, nitro, cyano, C₁-C₄-alkyl, C₂-C₄-alkenyl, C₁-C₄-haloalkyl, C₁-C₄-hydroxyalkyl, C₁-C₄-alkoxy, C₁-C₄-haloalkoxy, HO—(C₂-C₄-alkoxy)-, (C₁-C₄-alkoxy)-(C₂-C₄-alkoxy)-, (C₁-C₄-haloalkyl)-S—, R⁹R¹⁰N—, R⁸—C(O)—NH—, R⁸—C(O)—, R⁸—O—C(O)—, R⁹R¹⁰N—C(O)— or (C₁-C₄-alkyl)-SO₂—;
R^{11a} represents a group selected from hydrogen, C₃-C₆-cycloalkyl, morpholino, R^{9a}R^{10a}N—;
R^{9a}R^{10a}N—C(O)—; a 5- to 6-membered heteroaryl, which is optionally substituted with methyl or
represents:

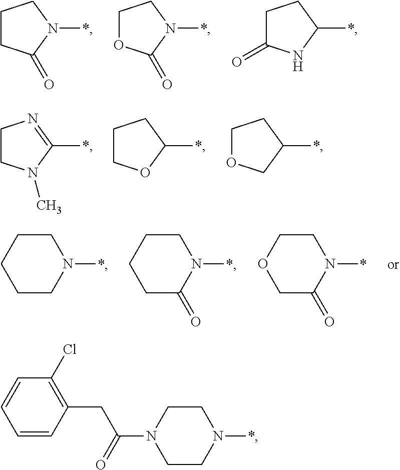

13. A compound according to claim 1 wherein
R² represents a group selected from: 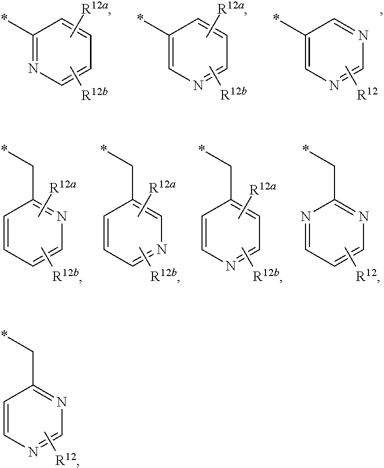
wherein * indicates the point of attachment of said group with the rest of the molecule
R¹² represents hydrogen, halogen, C₁-C₄-alkyl, C₃-C₆-cycloalkyl, methoxy, difluoromethyl or trifluoromethyl;
R^{12a} and R^{12b} represent, independently from each other, hydrogen, halogen, C₁-C₄-alkyl, C₃-C₆-cycloalkyl, methoxy, difluoromethyl or trifluoromethyl.

14. A compound according to claim 1 wherein
R² represents a group selected from: 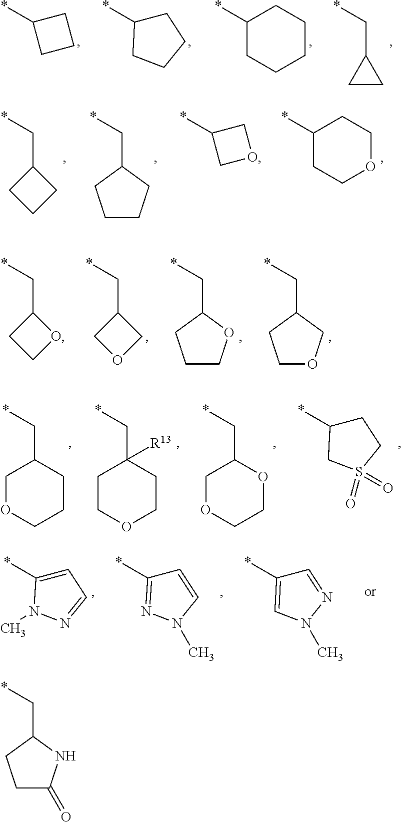
wherein * indicates the point of attachment of said group with the rest of the molecule;
R¹³ represents hydrogen, halogen, cyano or C₁-C₄-alkyl.

15. A compound according to claim 1 wherein
R⁵, R^{5a} and R^{5b} are the same or different and represent, independently from each other, hydrogen, halogen, C₁-C₄-alkyl, C₁-C₄-haloalkyl, C₁-C₄-alkoxy or C₁-C₄-haloalkoxy.

16. A compound according to claim 1 wherein
R⁸ represents C₁-C₄-alkyl, C₃-C₆-cycloalkyl or C₁-C₄-haloalkyl.

17. A compound according to claim 1 wherein
R⁹ represents, independently from each other, C₁-C₄-alkyl or C₃-C₆-cycloalkyl;
R¹⁰ represents, independently from each other, hydrogen or C₁-C₄-alkyl.

18. A compound according to claim 1 wherein
R^{9a} and R^{10a} together with the nitrogen atom to which they are attached form a 4- to 6-membered nitrogen containing heterocyclic ring, optionally containing one additional heteroatom selected from O, NMe or NH.

19. A compound according to claim 1 with a formula selected from the group consisting of:
N-[4-(3-chloro-5-cyanophenoxy)-3-sulfamoylphenyl]-2-(2-chlorophenyl)acetamide;
2-(2-chlorophenyl)-N-4-[3-(dimethylamino)phenoxy]-3-sulfamoylphenylacetamide;
2-(2-chlorophenyl)-N-4-[(2-chloropyridin-4-yl)oxy]-3-sulfamoylphenylacetamide;
2-(2-chlorophenyl)-N-[4-(3-isopropylphenoxy)-3-sulfamoylphenyl]acetamide;
2-(2-chlorophenyl)-N-3-sulfamoyl-4-[3-(trifluoromethyl)phenoxy]phenylacetamide;
2-(2-chlorophenyl)-N-3-sulfamoyl-4-[3-(trifluoromethoxy)phenoxy]phenylacetamide;
N-[4-(3-acetylphenoxy)-3-sulfamoylphenyl]-2-(2-chlorophenyl)acetamide;
N-[4-(1,3-benzodioxol-5-yloxy)-3-sulfamoylphenyl]-2-(2-chlorophenyl)acetamide;
N-[4-(3-acetamidophenoxy)-3-sulfamoylphenyl]-2-(2-chlorophenyl)acetamide;
2-(2-chlorophenyl)-N-[4-(2-fluorophenoxy)-3-sulfamoylphenyl]acetamide;
2-(2-chlorophenyl)-N-[4-(3-fluorophenoxy)-3-sulfamoylphenyl]acetamide;
2-(2-chlorophenyl)-N-[4-(4-fluorophenoxy)-3-sulfamoylphenyl]acetamide;
2-(2-chlorophenyl)-N-[4-(pyridin-2-yloxy)-3-sulfamoylphenyl]acetamide;
2-(2-chlorophenyl)-N-(4-phenoxy-3-sulfamoylphenyl)acetamide;
2-(2-chlorophenyl)-N-[4-(3-cyanophenoxy)-3-sulfamoylphenyl]acetamide;
2-(2-chlorophenyl)-N-4-[3-(methylsulfonyl)phenoxy]-3-sulfamoylphenylacetamide;
3-(4-[(2-chlorophenyl)acetyl]amino-2-sulfamoylphenoxy)benzamide;
2-(2-chlorophenyl)-N-[4-(3-methylphenoxy)-3-sulfamoylphenyl]acetamide;
2-(2-chlorophenyl)-N-[4-(pyrimidin-5-yloxy)-3-sulfamoylphenyl]acetamide;
2-(2-chlorophenyl)-N-3-sulfamoyl-4-[3-(4H-1,2,4-triazol-4-yl)phenoxy]phenylacetamide;
2-(2-chlorophenyl)-N-3-sulfamoyl-4-[3-(1H-tetrazol-5-yl)phenoxy]phenylacetamide;
2-(2-chlorophenyl)-N-[4-(3-methoxyphenoxy)-3-sulfamoylphenyl]acetamide;
2-(2-chlorophenyl)-N-[4-(4-methoxyphenoxy)-3-sulfamoylphenyl]acetamide;
2-(2-chlorophenyl)-N-4-[3-(difluoromethoxy)phenoxy]-3-sulfamoylphenylacetamide;
2-(2-chlorophenyl)-N-[4-(3,4-dicyanophenoxy)-3-sulfamoylphenyl]acetamide;
2-(2-chlorophenyl)-N-4-[3-(morpholin-4-yl)phenoxy]-3-sulfamoylphenylacetamide;
2-(2-chlorophenyl)-N-[4-(3-4-[(2-chlorophenyl)acetyl]piperazin-1-ylphenoxy)-3-sulfamoylphenyl]acetamide;
2-(2-chlorophenyl)-N-[4-(pyridin-3-yloxy)-3-sulfamoylphenyl]acetamide;
2-(2-chlorophenyl)-N-4-[(5-chloropyridin-3-yl)oxy]-3-sulfamoylphenylacetamide;
2-(2-chlorophenyl)-N-[4-(4-cyanophenoxy)-3-sulfamoylphenyl]acetamide;
2-(2-chlorophenyl)-N-4-[3-(difluoromethyl)phenoxy]-3-sulfamoylphenylacetamide;
2-(2-chlorophenyl)-N-[4-(2-methoxyphenoxy)-3-sulfamoylphenyl]acetamide;
2-(2-chlorophenyl)-N-[4-(3,5-dicyanophenoxy)-3-sulfamoylphenyl]acetamide;
2-(2-chlorophenyl)-N-[4-(5-cyano-2-methoxyphenoxy)-3-sulfamoylphenyl]acetamide;
2-(2-chlorophenyl)-N-4-[(2,5-dichloropyridin-3-yl)oxy]-3-sulfamoylphenylacetamide;
2-(2-chlorophenyl)-N-4-[(5,6-dichloropyridin-3-yl)oxy]-3-sulfamoylphenylacetamide;
3-(4-[(2-chlorophenyl)acetyl]amino-2-sulfamoylphenoxy)-N-cyclopropylbenzamide;
2-(2-chlorophenyl)-N-4-[(3-chloropyridin-2-yl)oxy]-3-sulfamoylphenylacetamide;
2-(2-chlorophenyl)-N-4-[(4-chloropyridin-2-yl)oxy]-3-sulfamoylphenylacetamide;
2-(2-chlorophenyl)-N-4-[(6-chloropyridin-2-yl)oxy]-3-sulfamoylphenylacetamide;
2-(2-chlorophenyl)-N-4-[3-(1-methyl-4,5-dihydro-1H-imidazol-2-yl)phenoxy]-3-sulfamoylphenylacetamide;
2-(2-chlorophenyl)-N-4-[4-(1H-imidazol-1-yl)phenoxy]-3-sulfamoylphenylacetamide;
2-(2-chlorophenyl)-N-4-[4-(2-oxopyrrolidin-1-yl)phenoxy]-3-sulfamoylphenylacetamide;
2-(2-chlorophenyl)-N-4-[4-(morpholin-4-yl)phenoxy]-3-sulfamoylphenylacetamide;
2-(2-chlorophenyl)-N-[4-(5-cyano-2-methylphenoxy)-3-sulfamoylphenyl]acetamide;
2-(2-chlorophenyl)-N-[4-(3-cyano-2-methylphenoxy)-3-sulfamoylphenyl]acetamide;
2-(2-chlorophenyl)-N-[4-(3-cyano-4-fluorophenoxy)-3-sulfamoylphenyl]acetamide;
N-4-[(5-chloro-2-cyanopyridin-3-yl)oxy]-3-sulfamoylphenyl-2-(2-chlorophenyl)acetamide;
2-(2-chlorophenyl)-N-4-[3-(piperidin-1-yl)phenoxy]-3-sulfamoylphenylacetamide;
2-(2-chlorophenyl)-N-4-[3-(2-oxopyrrolidin-1-yl)phenoxy]-3-sulfamoylphenylacetamide;
2-(2-chlorophenyl)-N-4-[3-(2-oxo-1,3-oxazolidin-3-yl)phenoxy]-3-sulfamoylphenylacetamide;
2-(2-chlorophenyl)-N-4-[3-(morpholin-4-ylcarbonyl)phenoxy]-3-sulfamoylphenylacetamide;
2-(2-chlorophenyl)-N-4-[(4-methyltetrahydro-2H-pyran-4-yl)methoxy]-3-sulfamoylphenylacetamide;
2-(2-chlorophenyl)-N-4-[(4-fluorotetrahydro-2H-pyran-4-yl)methoxy]-3-sulfamoylphenylacetamide;
2-(2-chlorophenyl)-N-4-[(4-cyanotetrahydro-2H-pyran-4-yl)methoxy]-3-sulfamoylphenylacetamide;
2-(2-chlorophenyl)-N-(3-sulfamoyl-4-[2-(trifluoromethyl)pyrimidin-5-yl]oxyphenyl)acetamide;
2-(2-chlorophenyl)-N-4-[(2-isopropylpyrimidin-5-yl)oxy]-3-sulfamoylphenylacetamide;
2-(2-chlorophenyl)-N-4-[(2-cyclopropyl-4-methylpyrimidin-5-yl)oxy]-3-sulfamoylphenylacetamide;
N-[4-(3-bromophenoxy)-3-sulfamoylphenyl]-2-(2-chlorophenyl)acetamide;
N-[4-(4-bromophenoxy)-3-sulfamoylphenyl]-2-(2-chlorophenyl)acetamide;
2-(2-chlorophenyl)-N-4-[3-(2-methyl-1,3-thiazol-4-yl)phenoxy]-3-sulfamoylphenylacetamide;
2-(2-chlorophenyl)-N-4-[4-(5-oxopyrrolidin-2-yl)phenoxy]-3-sulfamoylphenylacetamide;
2-(2-chlorophenyl)-N-4-[4-(2-oxo-1,3-oxazolidin-3-yl)phenoxy]-3-sulfamoylphenylacetamide;
2-(2-chlorophenyl)-N-3-sulfamoyl-4-[4-(1,3-thiazol-2-yl)phenoxy]phenylacetamide;
N-[4-(2-chlorophenoxy)-3-sulfamoylphenyl]-2-(2-chlorophenyl)acetamide;
N-[4-(4-chlorophenoxy)-3-sulfamoylphenyl]-2-(2-chlorophenyl)acetamide;
2-(2-chlorophenyl)-N-4-[3-(piperidin-1-ylcarbonyl)phenoxy]-3-sulfamoylphenylacetamide;
2-(2-chlorophenyl)-N-3-sulfamoyl-4-[4-(tetrahydrofuran-3-yl)phenoxy]phenylacetamide;
2-(2-chlorophenyl)-N-[4-(3-cyano-5-fluorophenoxy)-3-sulfamoylphenyl]acetamide;
N-[4-(2-methoxyphenoxy)-3-sulfamoylphenyl]-2-phenylacetamide;
N-[4-(2-methoxyphenoxy)-3-sulfamoylphenyl]-2-[4-(trifluoromethyl)phenyl]acetamide;
N-3-sulfamoyl-4-[2-(trifluoromethoxy)phenoxy]phenyl-2-[4-(trifluoromethyl)phenyl]acetamide;
N-[4-(2-chlorophenoxy)-3-sulfamoylphenyl]-2-[4-(trifluoromethyl)phenyl]acetamide;
2-phenyl-N-3-sulfamoyl-4-[2-(trifluoromethoxy)phenoxy]phenylacetamide;
2-(2-chlorophenyl)-N-4-[(2-oxo-1,2-dihydropyridin-3-yl)oxy]-3-sulfamoylphenylacetamide;
N-[4-(2-chlorophenoxy)-3-sulfamoylphenyl]-2-phenylacetamide;
N-[4-(4-chlorophenoxy)-3-sulfamoylphenyl]-2-phenylacetamide;
N-4-[(5-chloropyridin-3-yl)oxy]-3-sulfamoylphenyl-2-phenylacetamide;
2-(2-chlorophenyl)-N-4-[(2-chloropyrimidin-5-yl)oxy]-3-sulfamoylphenylacetamide;
2-(2-chlorophenyl)-N-4-[(5-fluoropyridin-3-yl)oxy]-3-sulfamoylphenylacetamide;
2-(2-chlorophenyl)-N-4-[(6-chloropyridin-3-yl)oxy]-3-sulfamoylphenylacetamide;
N-[2-chloro-4-(3-chlorophenoxy)-5-sulfamoylphenyl]-2-(2-chlorophenyl)acetamide;
N-[2-chloro-4-(3-chlorophenoxy)-5-sulfamoylphenyl]-2-(2-chloro-3-fluorophenyl)acetamide;
N-[4-(3-chlorophenoxy)-3-sulfamoylphenyl]-2-(3-fluorophenyl)acetamide;
N-[4-(3-chlorophenoxy)-3-sulfamoylphenyl]-2-(4-fluorophenyl)acetamide;
N-[4-(3-chlorophenoxy)-3-sulfamoylphenyl]-2-[2-(trifluoromethyl)phenyl]acetamide;
N-[4-(3-chlorophenoxy)-3-sulfamoylphenyl]-2-(2-isopropylphenyl)acetamide;
N-[4-(3-chlorophenoxy)-3-sulfamoylphenyl]-2-(2-ethoxyphenyl)acetamide;
N-[4-(3-chlorophenoxy)-3-sulfamoylphenyl]-2-[2-(difluoromethyl)phenyl]acetamide;
N-[4-(3-chlorophenoxy)-3-sulfamoylphenyl]-2-2-[(trifluoromethyl)sulfanyl]phenylacetamide;
2-(2-bromophenyl)-N-[4-(3-chlorophenoxy)-3-sulfamoylphenyl]acetamide;
N-[4-(3-chlorophenoxy)-3-sulfamoylphenyl]-2-(4-methylpyridin-3-yl)acetamide;
N-[4-(3-chlorophenoxy)-3-sulfamoylphenyl]-2-(2-chloropyridin-3-yl)acetamide;
N-[4-(3-chlorophenoxy)-3-sulfamoylphenyl]-2-(2-chlorophenyl)-2,2-difluoroacetamide;
2-(2-chloro-4-methylphenyl)-N-[4-(3-chlorophenoxy)-3-sulfamoylphenyl]acetamide;
2-(2-chloro-6-methylphenyl)-N-[4-(3-chlorophenoxy)-3-sulfamoylphenyl]acetamide;
2-(2-chloro-5-methylphenyl)-N-[4-(3-chlorophenoxy)-3-sulfamoylphenyl]acetamide;
2-(2-chloro-3-fluorophenyl)-N-[4-(3-chlorophenoxy)-3-sulfamoylphenyl]acetamide;
2-(2-chloro-5-fluorophenyl)-N-[4-(3-chlorophenoxy)-3-sulfamoylphenyl]acetamide;
2-(2-chloro-6-fluorophenyl)-N-[4-(3-chlorophenoxy)-3-sulfamoylphenyl]acetamide;
2-(2-chloro-6-methoxyphenyl)-N-[4-(3-chlorophenoxy)-3-sulfamoylphenyl]acetamide;
2-(2-chloro-5-methoxyphenyl)-N-[4-(3-chlorophenoxy)-3-sulfamoylphenyl]acetamide;
N-[4-(3-chlorophenoxy)-3-sulfamoylphenyl]-2-(2,3-dichlorophenyl)acetamide;
N-[4-(3-chlorophenoxy)-3-sulfamoylphenyl]-2-(2,6-dichlorophenyl)acetamide;
N-[4-(3-chlorophenoxy)-3-sulfamoylphenyl]-2-[2-(trifluoromethoxy)phenyl]acetamide;
N-[4-(3-chlorophenoxy)-3-sulfamoylphenyl]-2,2-difluoro-2-phenylacetamide;
N-[4-(3-chlorophenoxy)-3-sulfamoylphenyl]-2-[2-chloro-3-(trifluoromethyl)phenyl]acetamide;
N-[4-(3-chlorophenoxy)-3-sulfamoylphenyl]-2-[2-chloro-6-(trifluoromethyl)phenyl]acetamide;
N-[4-(3-chlorophenoxy)-3-sulfamoylphenyl]-2-[2-chloro-5-(trifluoromethyl)phenyl]acetamide;
N-[4-(3-chlorophenoxy)-3-sulfamoylphenyl]-2-(2,4-dichlorophenyl)acetamide;
N-[4-(3-chlorophenoxy)-3-sulfamoylphenyl]-2-(4,6-dichloropyridin-3-yl)acetamide;
N-[4-(3-chlorophenoxy)-3-sulfamoylphenyl]-2-(3-chloropyridin-2-yl)acetamide;
N-[4-(3-chlorophenoxy)-3-sulfamoylphenyl]-2-[2-(difluoromethoxy)phenyl]acetamide;
N-[4-(3-chlorophenoxy)-3-sulfamoylphenyl]-2-(2,5-dichlorophenyl)acetamide;
2-[6-chloro-2,3-difluoro-4-(trifluoromethyl)phenyl]-N-[4-(3-chlorophenoxy)-3-sulfamoylphenyl]acetamide;
N-[4-(3-chlorophenoxy)-3-sulfamoylphenyl]-2-[4-(trifluoromethyl)phenyl]acetamide;
2-(5-bromo-2-chlorophenyl)-N-[4-(3-chlorophenoxy)-3-sulfamoylphenyl]acetamide;
2-(4-bromo-2-chloro-5-methylphenyl)-N-[4-(3-chlorophenoxy)-3-sulfamoylphenyl]acetamide;
N-[4-(3-chlorophenoxy)-3-sulfamoylphenyl]-2-(3-chloropyridin-4-yl)acetamide;
2-(2-chloro-6-fluoro-3-methylphenyl)-N-[4-(3-chlorophenoxy)-3-sulfamoylphenyl]acetamide;
2-(6-chloro-2-fluoro-3-methylphenyl)-N-[4-(3-chlorophenoxy)-3-sulfamoylphenyl]acetamide;
2-(2-chloro-3,6-difluorophenyl)-N-[4-(3-chlorophenoxy)-3-sulfamoylphenyl]acetamide;
2-(2-chloro-4,5-difluorophenyl)-N-[4-(3-chlorophenoxy)-3-sulfamoylphenyl]acetamide;
N-[4-(3-chlorophenoxy)-3-sulfamoylphenyl]-2-(2,3-dichloro-6-fluorophenyl)acetamide;
N-[4-(3-chlorophenoxy)-3-sulfamoylphenyl]-2-(2,3,6-trichlorophenyl)acetamide;
N-[4-(3-chlorophenoxy)-3-sulfamoylphenyl]-2-(2,6-dichloro-4-methylphenyl)acetamide;
N-[4-(3-chlorophenoxy)-3-sulfamoylphenyl]-2-[2,3-dichloro-6-(trifluoromethyl)phenyl]acetamide;
N-[4-(3-chlorophenoxy)-3-sulfamoylphenyl]-2-(2,6-dichloro-3-methylphenyl)acetamide;
N-[4-(3-chlorophenoxy)-3-sulfamoylphenyl]-2-(2,6-dichloro-3-cyclopropylphenyl)acetamide;
N-[4-(3-chlorophenoxy)-3-sulfamoylphenyl]-2-[2,6-dichloro-3-(trifluoromethyl)phenyl]acetamide;
2-(3-bromo-2,6-dichlorophenyl)-N-[4-(3-chlorophenoxy)-3-sulfamoylphenyl]acetamide;
2-(3-bromo-2-chloro-6-methylphenyl)-N-[4-(3-chlorophenoxy)-3-sulfamoylphenyl]acetamide;
2-(3-bromo-6-chloro-2-methylphenyl)-N-[4-(3-chlorophenoxy)-3-sulfamoylphenyl]acetamide;
N-[4-(3-chlorophenoxy)-3-sulfamoylphenyl]-2-[2-chloro-5-(1,1,2,2-tetrafluoroethoxy)phenyl]acetamide;
N-[4-(3-chlorophenoxy)-3-sulfamoylphenyl]-2-[2-chloro-4-(trifluoromethyl)phenyl]acetamide;
2-(2-chlorophenyl)-N-(4-[3-(methylsulfonyl)benzyl]oxy-3-sulfamoylphenyl)acetamide;
2-(2-chlorophenyl)-N-4-[(2-fluorobenzyl)oxy]-3-sulfamoylphenylacetamide;
2-(2-chlorophenyl)-N-4-[(4-cyanobenzyl)oxy]-3-sulfamoylphenylacetamide;
N-4-[(3-chlorobenzyl)oxy]-3-sulfamoylphenyl-2-(2-chlorophenyl)acetamide;
2-(2-chlorophenyl)-N-4-[(3-methoxybenzyl)oxy]-3-sulfamoylphenylacetamide;
N-[4-(benzyloxy)-3-sulfamoylphenyl]-2-(2-chlorophenyl)acetamide;
2-(2-chlorophenyl)-N-4-[(3-cyanobenzyl)oxy]-3-sulfamoylphenylacetamide;
2-(2-chlorophenyl)-N-4-[(4-fluorobenzyl)oxy]-3-sulfamoylphenylacetamide;
N-4-[(2-chlorobenzyl)oxy]-3-sulfamoylphenyl-2-(2-chlorophenyl)acetamide;
2-(2-chlorophenyl)-N-4-[(2-cyanobenzyl)oxy]-3-sulfamoylphenylacetamide;
N-[4-(benzyloxy)-3-sulfamoylphenyl]-2-phenylacetamide;
2-(2-chlorophenyl)-N-(4-[4-(methylsulfonyl)benzyl]oxy-3-sulfamoylphenyl)acetamide;
2-(2-chlorophenyl)-N-[4-(1-phenylethoxy)-3-sulfamoylphenyl]acetamide;
2-(2-chlorophenyl)-N-[4-(1-phenylethoxy)-3-sulfamoylphenyl]acetamide;
2-(2-chlorophenyl)-N-[4-(pyridin-3-ylmethoxy)-3-sulfamoylphenyl]acetamide;
2-(2-chlorophenyl)-N-[4-(pyridin-2-ylmethoxy)-3-sulfamoylphenyl]acetamide;
2-(2-chlorophenyl)-N-[4-(pyridin-4-ylmethoxy)-3-sulfamoylphenyl]acetamide;
N-[4-(pyridin-2-ylmethoxy)-3-sulfamoylphenyl]-2-[4-(trifluoromethyl)phenyl]acetamide;
2-(2-chlorophenyl)-N-[4-(pyrimidin-4-ylmethoxy)-3-sulfamoylphenyl]acetamide;
2-(2-chlorophenyl)-N-[4-(pyrimidin-2-ylmethoxy)-3-sulfamoylphenyl]acetamide;
2-(2-chlorophenyl)-N-[4-(2-phenylethoxy)-3-sulfamoylphenyl]acetamide;
2-(2-chlorophenyl)-N-4-[2-(3-chlorophenyl)ethoxy]-3-sulfamoylphenyl acetamide;
2-(2-chlorophenyl)-N-[4-(cyclobutylmethoxy)-3-sulfamoylphenyl]acetamide;
2-(2-chlorophenyl)-N-[4-(oxetan-2-ylmethoxy)-3-sulfamoylphenyl]acetamide;
2-(2-chlorophenyl)-N-[4-(oxetan-3-ylmethoxy)-3-sulfamoylphenyl]acetamide;
2-(2-chlorophenyl)-N-[4-(cyclopentylmethoxy)-3-sulfamoylphenyl]acetamide;
2-(2-chlorophenyl)-N-[3-sulfamoyl-4-(tetrahydrofuran-2-ylmethoxy)phenyl]acetamide;
2-(2-chlorophenyl)-N-[3-sulfamoyl-4-(tetrahydrofuran-3-ylmethoxy)phenyl]acetamide;
2-(2-chloro-5-fluorophenyl)-N-[3-sulfamoyl-4-(tetrahydro-2H-pyran-4-ylmethoxy)phenyl]acetamide;
2-(2-chlorophenyl)-N-[3-sulfamoyl-4-(tetrahydro-2H-pyran-4-ylmethoxy)phenyl]acetamide;
2-(2-chlorophenyl)-N-[3-sulfamoyl-4-(tetrahydro-2H-pyran-3-ylmethoxy)phenyl]acetamide;
2-(2-chloro-6-fluorophenyl)-N-[3-sulfamoyl-4-(tetrahydro-2H-pyran-4-ylmethoxy)phenyl]acetamide;
2-(2-chloro-3-fluorophenyl)-N-[3-sulfamoyl-4-(tetrahydro-2H-pyran-4-ylmethoxy)phenyl]acetamide;
2-(2-chlorophenyl)-N-5-sulfamoyl-6-[3-(trifluoromethyl)phenoxy]pyridin-3-ylacetamide;
2-phenyl-N-5-sulfamoyl-6-[3-(trifluoromethyl)phenoxy]pyridin-3-ylacetamide;
N-[4-(3-chlorophenoxy)-3-fluoro-5-sulfamoylphenyl]-2-phenylacetamide;
N-[4-(3-chlorophenoxy)-3-fluoro-5-sulfamoylphenyl]-2-(2-methylphenyl)acetamide;
N-[4-(3-chlorophenoxy)-3-fluoro-5-sulfamoylphenyl]-2-(3-methylphenyl)acetamide;
2-(2-chlorophenyl)-N-4-[3-(3-oxomorpholin-4-yl)phenoxy]-3-sulfamoylphenylacetamide;
2-(2-chlorophenyl)-N-4-[4-(3-oxomorpholin-4-yl)phenoxy]-3-sulfamoylphenylacetamide;
2-(2-chlorophenyl)-N-4-[4-(2-oxopiperidin-1-yl)phenoxy]-3-sulfamoylphenylacetamide;
2-(2-chlorophenyl)-N-4-[3-(2-oxopiperidin-1-yl)phenoxy]-3-sulfamoylphenylacetamide;
2-(2-chlorophenyl)-N-4-[3-(prop-1-en-2-yl)phenoxy]-3-sulfamoylphenylacetamide;
2-(2-chlorophenyl)-N-4-[2-(prop-1-en-2-yl)phenoxy]-3-sulfamoylphenylacetamide;
N-[4-(3-chlorophenoxy)-3-sulfamoylphenyl]-2-(4-methylphenyl)acetamide;
N-[4-(3-chlorophenoxy)-3-sulfamoylphenyl]-2-(4-chlorophenyl)acetamide;
N-[4-(3-chlorophenoxy)-3-sulfamoylphenyl]-2-(pyridin-3-yl)acetamide;
N-[4-(3-chlorophenoxy)-3-sulfamoylphenyl]-2-(2-methylphenyl)acetamide;
N-[4-(3-chlorophenoxy)-3-sulfamoylphenyl]-2-(3-methylphenyl)acetamide;
N-[4-(3-chlorophenoxy)-3-sulfamoylphenyl]-2-phenylpropanamide;
N-[4-(3-chlorophenoxy)-3-sulfamoylphenyl]-2-(pyridin-2-yl)acetamide;
N-[4-(3-chlorophenoxy)-3-sulfamoylphenyl]-2-(3-chlorophenyl)acetamide;
N-[4-(3-chlorophenoxy)-3-sulfamoylphenyl]-2-(2-chlorophenyl)acetamide;
N-[4-(3-chlorophenoxy)-3-sulfamoylphenyl]-2-(pyridin-4-yl)acetamide;
N-[4-(3-chlorophenoxy)-3-sulfamoylphenyl]-2-(6-methylpyridin-2-yl)acetamide;
N-[4-(3-chlorophenoxy)-3-sulfamoylphenyl]-2-(4-methoxyphenyl)acetamide;
N-[4-(3-chlorophenoxy)-3-sulfamoylphenyl]-2-(3-methoxyphenyl)acetamide;
N-[4-(3-chlorophenoxy)-3-sulfamoylphenyl]-2-(2-methoxyphenyl)acetamide;
N-[4-(3-chlorophenoxy)-3-sulfamoylphenyl]-2-(5-methylpyridin-2-yl)acetamide;
(2S)—N-[4-(3-chlorophenoxy)-3-sulfamoylphenyl]-2-phenylpropanamide;
(2R)—N-[4-(3-chlorophenoxy)-3-sulfamoylphenyl]-2-phenylpropanamide;
N-[4-(3-chlorophenoxy)-3-sulfamoylphenyl]-2-(2-chlorophenyl)propanamide;
2-(2-[4-(3-chlorophenoxy)-3-sulfamoylphenyl]amino-2-oxoethyl)-N-(2-methoxyethyl)-N-methylbenzamide;
2-(2-[4-(3-chlorophenoxy)-3-sulfamoylphenyl]amino-2-oxoethyl)-N,N-dimethylbenzamide;
N-[4-(cyclohexyloxy)-3-sulfamoylphenyl]-2-phenylacetamide;
2-(2-chlorophenyl)-N-[4-(cyclohexyloxy)-3-sulfamoylphenyl]acetamide;
3-(2-[4-(3-chlorophenoxy)-3-sulfamoylphenyl]amino-2-oxoethyl)-N-(2-methoxyethyl)benzamide;
3-(2-[4-(3-chlorophenoxy)-3-sulfamoylphenyl]amino-2-oxoethyl)-N,N-dimethylbenzamide;
3-(2-[4-(3-chlorophenoxy)-3-sulfamoylphenyl]amino-2-oxoethyl)-N-methylbenzamide;
N-[4-(cyclobutyloxy)-3-sulfamoylphenyl]-2-phenylacetamide;
2-(2-chlorophenyl)-N-[4-(cyclobutyloxy)-3-sulfamoylphenyl]acetamide;
2-phenyl-N-[3-sulfamoyl-4-(tetrahydro-2H-pyran-4-yloxy)phenyl]acetamide;
2-(2-chlorophenyl)-N-[3-sulfamoyl-4-(tetrahydro-2H-pyran-4-yloxy)phenyl]acetamide;
3-(2-[4-(3-chlorophenoxy)-3-sulfamoylphenyl]amino-2-oxoethyl)-N-(2-methoxyethyl)-N-methylbenzamide;
N-[4-(3-chlorophenoxy)-3-sulfamoylphenyl]-2-(5-chloropyridin-2-yl)acetamide;
N-[4-(3-chlorophenoxy)-3-sulfamoylphenyl]-2-[3-(2-methoxyethoxy)phenyl]acetamide;
N-[4-(3-chlorophenoxy)-3-sulfamoylphenyl]-2-[2-(2-methoxyethoxy)phenyl]acetamide;
N-[4-(3-chlorophenoxy)-3-sulfamoylphenyl]-2-[3-(2-hydroxyethoxy)phenyl]acetamide;
N-[4-(3-chlorophenoxy)-3-sulfamoylphenyl]-2-[2-(2-hydroxyethoxy)phenyl]acetamide;
N-[4-(3-chlorophenoxy)-3-sulfamoylphenyl]-2-(2-fluorophenyl)acetamide;
N-[4-(oxetan-3-yloxy)-3-sulfamoylphenyl]-2-phenylacetamide;
2-(2-chlorophenyl)-N-[4-(oxetan-3-yloxy)-3-sulfamoylphenyl]acetamide;
N-[4-(cyclopentyloxy)-3-sulfamoylphenyl]-2-phenylacetamide;
2-(2-chlorophenyl)-N-[4-(cyclopentyloxy)-3-sulfamoylphenyl]acetamide;
N-4-[(1-methylpiperidin-3-yl)oxy]-3-sulfamoylphenyl-2-phenylacetamide;
2-(2-chlorophenyl)-N-4-[(1-methylpiperidin-3-yl)oxy]-3-sulfamoylphenylacetamide;
N-4-[(1-methylpyrrolidin-3-yl)oxy]-3-sulfamoylphenyl-2-phenylacetamide;
2-(2-chlorophenyl)-N-4-[(1-methylpyrrolidin-3-yl)oxy]-3-sulfamoylphenylacetamide;
N-[4-(4-chlorophenoxy)-3-sulfamoylphenyl]-2-(4-fluorophenyl)acetamide;
N-[4-(3-chlorophenoxy)-3-sulfamoylphenyl]-2-(4-cyanophenyl)acetamide;
N-[4-(3-chlorophenoxy)-3-sulfamoylphenyl]-2-(2-cyanophenyl)acetamide;
N-[4-(3-chlorophenoxy)-3-sulfamoylphenyl]-2-(3-cyanophenyl)acetamide;
N-[4-(4-chlorophenoxy)-3-sulfamoylphenyl]-2-[4-(trifluoromethyl)phenyl]acetamide;
N-[4-(4-chlorophenoxy)-3-sulfamoylphenyl]-2-(4-chlorophenyl)acetamide;
N-[4-(4-chlorophenoxy)-3-sulfamoylphenyl]-2-(4-methoxyphenyl)acetamide;
N-[4-(4-chlorophenoxy)-3-sulfamoylphenyl]-2-(2-fluorophenyl)acetamide;
2-(2-chloro-4-fluorophenyl)-N-[4-(4-chlorophenoxy)-3-sulfamoylphenyl]acetamide;
2-(2-chlorophenyl)-N-4-[(1,1-dioxidotetrahydrothiophen-3-yl)oxy]-3-sulfamoylphenylacetamide;
2-(2-chlorophenyl)-N-4-[(1-methyl-1H-pyrazol-4-yl)oxy]-3-sulfamoylphenylacetamide;
N-[4-(4-chlorophenoxy)-3-sulfamoylphenyl]-2-[4-(difluoromethyl)phenyl]acetamide;
2-(2-chloro-4-methoxyphenyl)-N-[4-(4-chlorophenoxy)-3-sulfamoylphenyl]acetamide;
2-(2-chlorophenyl)-N-4-[(1-methyl-1H-pyrazol-3-yl)oxy]-3-sulfamoylphenylacetamide;
2-(2-chlorophenyl)-N-4-[(1-methyl-1H-pyrazol-5-yl)oxy]-3-sulfamoylphenylacetamide;
2-(2-chlorophenyl)-N-4-[(1-methylpiperidin-4-yl)oxy]-3-sulfamoylphenylacetamide;
2-(2-chlorophenyl)-N-(4-[5-methyl-2-(pyridin-3-yl)-1,3-thiazol-4-yl]oxy-3-sulfamoylphenyl)acetamide;
N-[4-(3-chlorophenoxy)-2-methyl-5-sulfamoylphenyl]-2-(2-chlorophenyl)acetamide;
2-(2-chlorophenyl)-N-{4-[(1-oxidotetrahydrothiophen-3-yl)oxy]-3-sulfamoylphenyl}acetamide;
N-[4-(4-chlorophenoxy)-3-sulfamoylphenyl]-2-[2,6-dichloro-4-(trifluoromethyl)phenyl]acetamide;
N-[4-(4-chlorophenoxy)-3-sulfamoylphenyl]-2-(2,5-dichloro-4-cyanophenyl)acetamide;
N-[4-(3-chlorophenoxy)-3-sulfamoylphenyl]-2-phenylacetamide;
N-[4-(cyclopropylmethoxy)-3-sulfamoylphenyl]-2-phenylacetamide;
N-[4-(3,5-dimethylphenoxy)-3-sulfamoylphenyl]-2-phenylacetamide;
N-[4-(2,4-difluorophenoxy)-3-sulfamoylphenyl]-2-phenylacetamide;
N-[4-(4-fluorophenoxy)-3-sulfamoylphenyl]-2-phenylacetamide;
N-[4-(3-fluorophenoxy)-3-sulfamoylphenyl]-2-phenylacetamide;
N-[4-(3-methoxyphenoxy)-3-sulfamoylphenyl]-2-phenylacetamide;
N-[4-(2-fluoro-5-methylphenoxy)-3-sulfamoylphenyl]-2-phenylacetamide;
2-phenyl-N-3-sulfamoyl-4-[4-(trifluoromethoxy)phenoxy]phenylacetamide;
2-phenyl-N-3-sulfamoyl-4-[3-(trifluoromethyl)phenoxy]phenylacetamide;
N-[4-(3,5-dimethoxyphenoxy)-3-sulfamoylphenyl]-2-phenylacetamide;
N-[4-(3-cyanophenoxy)-3-sulfamoylphenyl]-2-phenylacetamide;
N-[4-(3-chlorophenoxy)-3-sulfamoylphenyl]-2-(4-hydroxyphenyl)acetamide;
2-(2-chloro-6-methoxy-4-methylphenyl)-N-[4-(3-chlorophenoxy)-3-sulfamoylphenyl]acetamide;
2-(2-chloro-6-fluorophenyl)-N-[4-(3-chlorophenoxy)-3-sulfamoylphenyl]propanamide;
2-(2-chloro-4,6-difluorophenyl)-N-[4-(3-chlorophenoxy)-3-sulfamoylphenyl]acetamide;
N-[4-(3-chlorophenoxy)-3-sulfamoylphenyl]-2-(2,6-dichlorophenyl)propanamide;
2-(2-chlorophenyl)-N-4-[(²H₅)phenyloxy]-3-sulfamoylphenylacetamide;
2-(2-chlorophenyl)-N-(4-{[4-chloro(²H₄)phenyl]oxy}-3-sulfamoylphenyl)acetamide;
2-(2-chlorophenyl)-N-(4-{[2-chloro(²H₄)phenyl]oxy}-3-sulfamoylphenyl)acetamide;
2-(2-chlorophenyl)-N-4-[4-(2-hydroxypropan-2-yl)phenoxy]-3-sulfamoylphenylacetamide;
2-(2-chlorophenyl)-N-4-[(2,2-dimethyltetrahydro-2H-pyran-4-yl)methoxy]-3-sulfamoylphenylacetamide;
2-(2-chlorophenyl)-N-{4-[(1R,5S,6r)-3-oxabicyclo[3.1.0]hex-6-ylmethoxy]-3-sulfamoylphenyl}acetamide;
2-(2-chlorophenyl)-N-4-[(4-chlorotetrahydro-2H-pyran-4-yl)methoxy]-3-sulfamoylphenylacetamide;
2-(2-chlorophenyl)-N-[4-(1,4-dioxan-2-ylmethoxy)-3-sulfamoylphenyl]acetamide;
2-(2-chlorophenyl)-N-3-sulfamoyl-4-[(2,2,6,6-tetramethyltetrahydro-2H-pyran-4-yl)oxy]phenylacetamide;
N-[4-(3-chlorophenoxy)-3-methyl-5-sulfamoylphenyl]-2-(2-chlorophenyl)acetamide;
N-[4-(3-chlorophenoxy)-3-methyl-5-sulfamoylphenyl]-2-phenylacetamide;
methyl 2-(4-[(2-chlorophenyl)acetyl]amino-2-sulfamoylphenoxy)benzoate;
methyl 4-(4-[(2-chlorophenyl)acetyl]amino-2-sulfamoylphenoxy)benzoate;
2-(2-chlorophenyl)-N-4-[3-(2-hydroxypropan-2-yl)phenoxy]-3-sulfamoylphenylacetamide;
2-(2-chlorophenyl)-N-4-[2-(2-hydroxypropan-2-yl)phenoxy]-3-sulfamoylphenylacetamide;
N-[4-(4-chlorophenoxy)-3-sulfamoylphenyl]-2-(2,3-dihydro-1,4-benzodioxin-6-yl)acetamide;
2-(7-chloro-2,3-dihydro-1,4-benzodioxin-6-yl)-N-[4-(3-chlorophenoxy)-3-sulfamoylphenyl]acetamide;
2-(5-chloro-2,3-dihydro-1-benzofuran-4-yl)-N-[4-(3-chlorophenoxy)-3-sulfamoylphenyl]acetamide;
2-(2-fluorophenyl)-N-[3-sulfamoyl-4-(tetrahydro-2H-pyran-4-ylmethoxy)phenyl]acetamide;
N-[3-sulfamoyl-4-(tetrahydro-2H-pyran-4-ylmethoxy)phenyl]-2-[2-(trifluoromethyl)phenyl]acetamide;
2-[2-(difluoromethyl)phenyl]-N-[3-sulfamoyl-4-(tetrahydro-2H-pyran-4-ylmethoxy)phenyl]acetamide;
2-(2-chloro-4-fluorophenyl)-N-[3-sulfamoyl-4-(tetrahydro-2H-pyran-4-ylmethoxy)phenyl]acetamide;
2-(2-Chlorophenyl)-N-(3-sulfamoyl-4-{[6-(trifluoromethyl)pyridin-3-yl]oxy})phenyl)-acetamide;
2-(2-Chlorophenyl)-N-(4-{[5-chloro-4-(trifluoromethyl)pyridin-2-yl]oxy}-3-sulfamoyl-phenyl)acetamide;
N-[4-(3-chlorophenoxy)-3-sulfamoylphenyl]-2-phenyl(2H2)acetamide;
N-{4-[(6-Chloro-5-fluoropyridin-3-yl)oxy]-3-sulfamoylphenyl)}-2-(2-chlorophenyl)-acetamide;
2-(2-Chlorophenyl)-N-{4-[(4,4-difluoro-1-hydroxycyclohexyl)methoxy]-3-sulfamoyl-phenyl}acetamide;
2-(2-Chlorophenyl)-N-{4-[(1-hydroxycyclohexyl)methoxy]-3-sulfamoylphenyl})-acetamide;
N-[4-(3-Chlorophenoxy)-3-fluoro-5-sulfamoylphenyl]-2-(2-fluorophenyl)acetamide;
N-[4-(3-chlorophenoxy)-3-fluoro-5-sulfamoylphenyl]-2-[2-(difluoromethyl)-phenyl]acetamide;
N-[4-(3-chlorophenoxy)-3-fluoro-5-sulfamoylphenyl]-2-(2-chlorophenyl)acetamide;
2-(2-chloro-5-fluorophenyl)-N-[4-(3-chlorophenoxy)-3-fluoro-5-sulfamoyl-phenyl]acetamide;
N-[6-(3-Chlorophenoxy)-5-sulfamoylpyridin-3-yl]-2-(2-fluorophenyl)acetamide;
N-[6-(3-Chlorophenoxy)-5-sulfamoylpyridin-3-yl]-2-[2-(trifluoromethyl)phenyl]-acetamide;
N-[6-(3-Chlorophenoxy)-5-sulfamoylpyridin-3-yl]-2-[2-(difluoromethyl)phenyl]-acetamide and 2-(2-Chloro-5-fluorophenyl)-N-[6-(3-chlorophenoxy)-5-sulfamoylpyridin-3-yl]-acetamide.

20. A compound according to claim 1 for the manufacture of a medicament.

21. A compound according to claim 1 for use in the treatment or prophylaxis of a disease wherein said disease is a genitourinary, gastrointestinal, proliferative or pain-related disease, condition or disorder; cancer; amyotrophic lateral sclerosis (ALS); fibrotic diseases including lung fibrosis, heart fibrosis, kidney fibrosis and fibrosis of other organs; gynaecological diseases including dysmenorrhea, dyspareunia, endometriosis and adenomyosis; endometriosis-associated pain; endometriosis-associated symptoms, wherein said symptoms are in particular endometriosis-associated proliferation, dysmenorrhea, dyspareunia, dysuria, or dyschezia; endometriosis-associated proliferation; pelvic hypersensitivity; urethritis; prostatitis; prostatodynia; cystitis; idiopathic bladder hypersensitivity; gastrointestinal disorders including irritable bowel syndrome (IBS), inflammatory bowel disease (IBD), biliary colic and other biliary disorders, renal colic, diarrhea-dominant IBS, gastroesophageal reflux, gastrointestinal distension, Crohn's disease and the like; atherosclerosis; lipid disorders; and pain-associated diseases selected from the group consisting of hyperalgesia, allodynia, functional bowel disorders (such as irritable bowel syndrome), arthritis (such as osteoarthritis and rheumatoid arthritis), burns, migraine or cluster headaches, nerve injury, neuritis, neuralgias, poisoning, ischemic injury, interstitial cystitis, cancer, traumatic nerve-injury, post-traumatic injuries (including fractures and sport injuries), trigeminal neuralgia, small fiber neuropathy, diabetic neuropathy, chronic arthritis and related neuralgias, HIV and HIV treatment-induced neuropathy, pruritus; impaired wound healing and disease of the skeleton like degeneration of the joints, ankylosing spondylitis.

22. A compound according to claim 1 for use in the treatment of pain syndromes (including acute, chronic, inflammatory and neuropathic pain), inflammatory pain, surgical pain, visceral pain, dental pain, premenstrual pain, endometriosis-associated pain, pain associated with fibrotic diseases, central pain, pain due to burning mouth syndrome, pain due to burns, pain due to migraine, cluster headaches, pain due to nerve injury, pain due to neuritis, neuralgias, pain due to poisoning, pain due to ischemic injury, pain due to interstitial cystitis, cancer pain, pain due to viral, parasitic or bacterial infections, pain due to traumatic nerve-injury, pain due to post-traumatic injuries (including fractures and sport injuries), pain due to trigeminal neuralgia, pain associated with small fiber neuropathy, pain associated with diabetic neuropathy, chronic lower back pain, phantom limb pain, pelvic pain syndrome, chronic pelvic pain, neuroma pain, complex regional pain syndrome, pain associated with gastrointestinal distension, chronic arthritic pain and related neuralgias, and pain associated with cancer, pain associated with chemotherapy, HIV and HIV treatment-induced neuropathy; and pain associated with diseases or disorders selected from the group consisting of hyperalgesia, allodynia, functional bowel disorders (such as irritable bowel syndrome) and arthritis (such as osteoarthritis and rheumatoid arthritis).

23. A compound according to claim 1 for use in the treatment of a gynecological disease, preferably dysmenorrhea, dyspareunia or endometriosis, adenomyosis, endometriosis-associated pain, or other endometriosis-associated symptoms, wherein said symptoms are in particular endometriosis-associated proliferation, dysmenorrhea, dyspareunia, dysuria or dyschezia.

24. A pharmaceutical composition comprising at least one compound according to claim 1, together with at least one pharmaceutically acceptable auxiliary.

25. Use of a compound according to claim 1 for the prophylaxis or treatment of a disease.

26. Use of a compound according to claim 1 for the preparation of a medicament for the prophylaxis or treatment of a disease, wherein said disease is a genitourinary, gastrointestinal, proliferative or pain-related disease, condition or disorder; cancer; amyotrophic lateral sclerosis (ALS); fibrotic diseases including lung fibrosis, heart fibrosis, kidney fibrosis and fibrosis of other organs; gynaecological diseases including dysmenorrhea, dyspareunia, endometriosis and adenomyosis; endometriosis-associated pain; endometriosis-associated symptoms, wherein said symptoms are in particular endometriosis-associated proliferation, dysmenorrhea, dyspareunia, dysuria, or dyschezia; endometriosis-associated proliferation; pelvic hypersensitivity; urethritis; prostatitis; prostatodynia; cystitis; idiopathic bladder hypersensitivity; gastrointestinal disorders including irritable bowel syndrome (BS), inflammatory bowel disease (IBD), biliary colic and other biliary disorders, renal colic, diarrhea-dominant IBS, gastroesophageal reflux, gastrointestinal distension, Crohn's disease and the like; atherosclerosis; lipid disorders; and pain-associated diseases selected from the group consisting of hyperalgesia, allodynia, functional bowel disorders (such as irritable bowel syndrome), arthritis (such as osteoarthritis and rheumatoid arthritis), burns, migraine or cluster headaches, nerve injury, neuritis, neuralgias, poisoning, ischemic injury, interstitial cystitis, cancer, traumatic nerve-injury, post-traumatic injuries (including fractures and sport injuries), trigeminal neuralgia, small fiber neuropathy, diabetic neuropathy, chronic arthritis and related neuralgias, HIV and HIV treatment-induced neuropathy, pruritus; impaired wound healing and disease of the skeleton like degeneration of the joints, ankylosing spondylitis; pain syndromes (including acute, chronic, inflammatory and neuropathic pain), inflammatory pain, surgical pain, visceral pain, dental pain, premenstrual pain, endometriosis-associated pain, pain associated with fibrotic diseases, central pain, pain due to burning mouth syndrome, pain due to burns, pain due to migraine, cluster headaches, pain due to nerve injury, pain due to neuritis, neuralgias, pain due to poisoning, pain due to ischemic injury, pain due to interstitial cystitis, cancer pain, pain due to viral, parasitic or bacterial infections, pain due to traumatic nerve-injury, pain due to post-traumatic injuries (including fractures and sport injuries), pain due to trigeminal neuralgia, pain associated with small fiber neuropathy, pain associated with diabetic neuropathy, chronic lower back pain, phantom limb pain, pelvic pain syndrome, chronic pelvic pain, neuroma pain, complex regional pain syndrome, pain associated with gastrointestinal distension, chronic arthritic pain and related neuralgias, and pain associated with cancer, pain associated with chemotherapy, HIV and HIV treatment-induced neuropathy; and pain associated with diseases or disorders selected from the group consisting of hyperalgesia, allodynia, functional bowel disorders (such as irritable bowel syndrome) and arthritis (such as osteoarthritis and rheumatoid arthritis); a gynecological disease, preferably dysmenorrhea, dyspareunia or endometriosis, adenomyosis, endometriosis-associated pain, or other endometriosis-associated symptoms, wherein said symptoms are in particular endometriosis-associated proliferation, dysmenorrhea, dyspareunia, dysuria or dyschezia.

27. An intermediate of formula 9 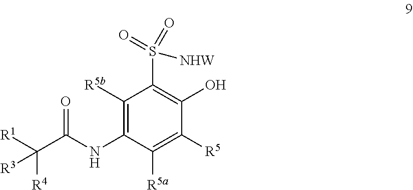
whereby R¹, R³, R⁴, R⁵, R^{5a} and R^{5b} are defined according to claim 1 and W corresponds to either a hydrogen atom or a protecting group.

28. An intermediate of formula 13 or 14 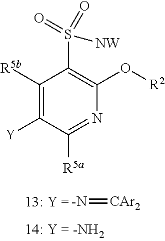
whereby R², R³, R⁴, R^{5a} and R^{5b} are defined according to claim 1 and W corresponds to either a hydrogen atom or a protecting group.

29. An intermediate of a formula selected from the group consisting of:
N-(2,4-Dimethoxybenzyl)-2-fluoro-5-nitrobenzenesulfonamide;
2,4-Dichloro-N-(2,4-dimethoxybenzyl)-5-nitrobenzenesulfonamide;
N-(2,4-Dimethoxybenzyl)-2,3-difluoro-5-nitrobenzenesulfonamide;
2-(2-Chlorophenyl)-N-(4-hydroxy-3-sulfamoylphenyl)acetamide;
2-(2-Chloro-3-fluorophenyl)-N-(4-hydroxy-3-sulfamoylphenyl)acetamide;
2-(2-Chloro-6-fluorophenyl)-N-(4-hydroxy-3-sulfamoylphenyl)acetamide;
5-Bromo-2-hydroxypyridine-3-sulfonamide;
5-Amino-2-[3-(trifluoromethyl)phenoxy]pyridine-3-sulfonamide;
N-(2,4-Dimethoxybenzyl)-2-fluoro-4-methyl-5-nitrobenzenesulfonamide; and
N-(2,4-Dimethoxybenzyl)-2-fluoro-3-methyl-5-nitrobenzenesulfonamide.
